(19) United States
(12) Patent Application Publication
(10) Pub. No.: US 2026/0308539 A1
(43) Pub. Date: Oct. 8, 2026

(54) MYOTIS LUCIFUGUS TRANSPOSASE ENGINEERING

(71) Applicants: Blue Marlin Therapeutics, Inc., Charlottesville, VA (US); University of Hawai'i, Honolulu, HI (US)

(72) Inventors: Joseph J. Higgins, Charlottesville, VA (US); Nancy Craig, Charlottesville, VA (US); Subhrangshu Guhathakurta, Charlottesville, VA (US); Jesse B. Owens, Honolulu, HI (US)

(73) Assignees: Blue Marlin Therapeutics, Inc., Charlottesville, VA (US); University of Hawai'i, Honolulu, HI (US)

(21) Appl. No.: 19/481,154

(22) PCT Filed: May 3, 2024
(86) PCT No.: PCT/US2024/027738

§ 371 Date: Nov. 3, 2025

Related U.S. Application Data
(60) Provisional application No. 63/500,172, filed on May 4, 2023.

(51) Int. Cl. C12N 9/12; C12N 9/22; C12N 15/11; C12N 15/85; C12N 15/88

(52) U.S. Cl. CPC C12N 9/1241; C12N 9/226; C12N 15/11; C12N 15/85; C12N 15/88; C12N 2310/20

(57) ABSTRACT

Compositions and methods related to recombinant mobile element systems and uses thereof are provided.

### CROSS-REFERENCE TO RELATED APPLICATIONS

This application claims priority to and the benefit of U.S. Provisional Patent Application No. 63/500,172, filed on May 4, 2023, the entire content of which are hereby incorporated herein by reference in its entirety.

### FIELD

The present disclosure relates to recombinant mobile element systems and uses thereof.

### SEQUENCE LISTING

The instant application contains a sequence listing, which has been submitted in XML format via EFS-Web. The contents of the XML copy named “SAL-031PC_126933-5031_Sequence Listing,” which was created on May 2, 2024 and is 925,696 bytes in size, the contents of which are incorporated herein by reference in their entirety.

### BACKGROUND

Mobile elements transposon systems are tools for genome engineering but unselected integration into the genome carries genotoxic risks in therapeutic applications. A nucleic acid movement to a new location in the human genome is performed by the action of a helper enzyme that binds to an “end sequence” and inserts a donor DNA sequence at a specific DNA sequence by a “cut and paste” mechanism. The donor DNA is flanked by end sequences in living organisms such as insects (e.g., Trichnoplusia ni). Genomic DNA is excised by double strand cleavage at the hosts' donor site and the donor DNA is integrated or inserted into a specific DNA sequence. Mobilization of the DNA sequences permits the intervening nucleic acid, or a transgene, to be inserted at the specific nucleotide sequence (i.e., TTAA) without a DNA footprint. Site-specific mutagenesis can also be improved by binding various DNA binding domains to transposon systems.

There is a need for compositions and methods for targeted site-directed integration.

### SUMMARY

Accordingly, this disclosure describes, in part, a helper DNA or RNA that encodes for an excision competent/integration defective (Exc+Int−) helper enzyme that is optionally engineered to target a single human genomic locus by introducing DNA binding proteins at its N-terminus or within internal DNA binding loops (Y281-T291 and I332-P339). The present disclosure provides a composition comprising a recombinant mobile element enzyme that has bioengineered enhanced gene cleavage [Excision (Exc+)] and/or integration deficient (Int−) and/or integration efficient (Int+) gene activity, and DNA binders (e.g., without limitation, dCas, TALEs, and ZnF) that guide donor insertion to specific genomic sites.

In aspects and there is provided a composition comprising the present disclosure provides a composition comprising a helper enzyme or a nucleic acid encoding the helper enzyme, an amino acid sequence having at least about 80% sequence identity to SEQ ID NO: 1 and a non-polar aliphatic amino acid at position 2 of SEQ ID NO: 1, or a position corresponding thereto, one or more amino acid substitutions selected from S8X, C13X, and D416X of SEQ ID NO: 1 or a substitution at a position corresponding thereto, and one or more amino acid substitutions selected from Y281X, C282X, G283X, E284X, G285X, K286X, R287X, L288X, L289X, E290X, T291X, I332X, R333X, K334X, N335X, R336X, G337X, I338X, and P339X of SEQ ID NO: 1 or a substitution at a position corresponding thereto.

In embodiments, the present disclosure provides a composition comprising a helper enzyme or a nucleic acid encoding the helper enzyme and a targeting element or a nucleic acid encoding the targeting element wherein, the helper enzyme comprises an amino acid sequence having at least about 80% sequence identity to SEQ ID NO: 1 and has a non-polar aliphatic amino acid at position 2 of SEQ ID NO: 1, or a position corresponding thereto, one or more amino acid substitutions selected from S8X, C13X, and D416X of SEQ ID NO: 1 or a substitution at a position corresponding thereto, and one or more amino acid substitutions selected from Y281X, C282X, G283X, E284X, G285X, K286X, R287X, L288X, L289X, E290X, T291X, I332X, R333X, K334X, N335X, R336X, G337X, I338X, and P339X of SEQ ID NO: 1 or a substitution at a position corresponding thereto.

In embodiments, the non-polar aliphatic amino acid is selected from alanine (A), glycine (G), valine (V), leucine (L), isoleucine (I), and proline (P).

In embodiments, X is selected from a non-polar amino acid, optionally selected from alanine (A), glycine (G), valine (V), leucine (L), isoleucine (I), proline (P), methionine (M), tryptophan (W), and phenylalanine (F), or a polar amino acid, optionally selected from serine(S), cysteine (C), asparagine (N), glutamine (Q), threonine (T), and tyrosine (Y), or a basic amino acid, optionally selected from arginine (R), histidine (H), or an acidic amino acid, optionally selected from aspartic acid (D), and glutamic acid (E), or a positively charged hydrophilic amino acid, optionally selected from lysine (K), arginine (R), and histidine (H).

In embodiments, the helper enzyme comprises a substitution at S8X of SEQ ID NO: 1 or a substitution at a position corresponding thereto, optionally wherein X is a non-polar residue, optionally selected from alanine (A), glycine (G), valine (V), leucine (L), isoleucine (I), or a polar and neutral hydrophilic residue, optionally proline (P).

In embodiments, the helper enzyme comprises a substitution at C13X of SEQ ID NO: 1 or a substitution at a position corresponding thereto, optionally wherein X is a positively charged hydrophilic amino acid, optionally selected from lysine (K), arginine (R), and histidine (H).

In embodiments, the helper enzyme comprises a substitution at D416X of SEQ ID NO: 1 or a substitution at a position corresponding thereto, wherein X is a polar and positively charged hydrophilic residue, optionally selected from arginine (R) and lysine (K), or a polar and neutral of charge hydrophilic residue, optionally selected from asparagine (N), glutamine (Q), serine(S), threonine (T), proline (P), and cysteine (C).

In embodiments, the helper enzyme comprises one, or two, or three, or four, or five, or six substitutions at positions selected from 2, 8, 13, 416, 282, 283, 284, 285, 286, 287, 288, 289, 290, 291, 332, 333, 334, 335, 336, 337, 338, and 339, or positions corresponding thereto relative to SEQ ID NO: 1.

In embodiments, the helper enzyme comprises one, or two, or three, or four substitutions at positions selected from: 2, 8, 13, 416, and one or two substitutions at positions selected from 281, 282, 283, 284, 285, 286, 287, 288, 289, 290, 291, 332, 333, 334, 335, 336, 337, 338, and 339, or positions corresponding thereto relative to SEQ ID NO: 1.

In embodiments, the helper enzyme comprises one or two, or three, or four substitutions selected from: S2A, S8P, C13R, D416A and D416N or substitutions corresponding thereto relative to SEQ ID NO: 1.

In embodiments, the help enzyme comprises a substitution selected from S8P, C13R, D416N, S8P/C13R, S8P/D416N, C13R/D416N, and S8P/C13R/D416N or substitutions corresponding thereto relative to SEQ ID NO: 1.

In embodiments, the helper enzyme comprises one or two substitutions selected from C282A, G283A, E284A, G285A, K286A, R287A, L288A, L289A, E290A, T291A, I332A, R333A, R333K, R333S, K334A, N335A, R336A, R336K, R336S, G337A, I338A, and P339A or substitutions corresponding thereto relative to SEQ ID NO: 1.

In embodiments, the composition comprises one or two, or three, or four substitutions selected from: S2A, S8P, C13R, D416A and D416N or substitutions corresponding thereto relative to SEQ ID NO: 1, and one or two substitutions selected from C282A, G283A, E284A, G285A, K286A, R287A, L288A, L289A, E290A, T291A, I332A, R333A, R333K, R333S, K334A, N335A, R336A, R336K, R336S, G337A, I338A, and P339A or substitutions corresponding thereto relative to SEQ ID NO: 1.

In embodiments, the composition one, or two, or three, or four, or five, or six substitutions selected from S2A, S8P, C13R, Y281A, C282A, G283A, E284A, G285A, K286A, R287A, L288A, L289A, E290A, T291A, I332A, R333A, R333K, R333S, K334A, N335A, R336A, R336K, R336S, G337A, I338A, P339A, D416A and D416N or substitutions corresponding thereto relative to SEQ ID NO: 1.

In embodiments, the helper enzyme comprises a substitution selected from TABLE 1, TABLE 1A, TABLE 2 and/or TABLE 2A or a substitution at positions corresponding thereto relative to SEQ ID NO: 1.

In embodiments, the helper enzyme comprises S8P/C13R/D416N/R333K/K334A/N335A/R336K, S8P/C13R/D416N/R333K/N335A/R336K, D416N/I338A, D416N/R333K, D416N/R333K/R336K, D416N/R336S, D416N/R333K/K334A, D416N/R333K/K334A/R336K, D416N/R333K/N335A, D416N/R333K/N335A/R336K, D416N/R333K/K334A/N335A, D416N/R333K/K334A/N335A/R336K, S8P/C13R/D416N/Y281A, S8P/C13R/D416N/K334A, S8P/C13R/D416N/K334A/N335A, S8P/C13R/D416N/K334A/R336A, S8P/C13R/D416N/N335A, S8P/C13R/D416N/N335A/R336A, S8P/C13R/D416N/R333A, S8P/C13R/D416N/R333A/K334A, S8P/C13R/D416N/R333A/N335A, S8P/C13R/D416N/R333A/R336A, S8P/C13R/D416N/R336A, S8P/C13R/D416N/Y281A/K334A, S8P/C13R/D416N/Y281A/K334A/N335A, S8P/C13R/D416N/K334A/N335A/R336K, S8P/C13R/D416N/K334A/N335A/R336S, S8P/C13R/D416N/K334A/R336K, S8P/C13R/D416N/K334A/R336S, S8P/C13R/D416N/N335A/R336K, S8P/C13R/D416N/N335A/R336S, S8P/C13R/D416N/R333K, S8P/C13R/D416N/R333K/K334A, S8P/C13R/D416N/R333K/K334A/N335A, S8P/C13R/D416N/R333K/K334A/N335A/R336S, S8P/C13R/D416N/R333K/K334A/R336A, S8P/C13R/D416N/R333K/K334A/R336K, S8P/C13R/D416N/R333K/K334A/R336S, S8P/C13R/D416N/R333K/N335A, S8P/C13R/D416N/R333K/N335A/R336S, S8P/C13R/D416N/R333K/R336K, S8P/C13R/D416N/R333K/R336S, S8P/C13R/D416N/R333S, S8P/C13R/D416N/R333S/K334A, S8P/C13R/D416N/R333S/K334A/N335A, S8P/C13R/D416N/R333S/K334A/N335A/R336K, S8P/C13R/D416N/R333S/K334A/N335A/R336S, S8P/C13R/D416N/R333S/K334A/R336K, S8P/C13R/D416N/R333S/K334A/R336S, S8P/C13R/D416N/R333S/N335A, S8P/C13R/D416N/R333S/N335A/R336K, S8P/C13R/D416N/R333S/N335A/R336S, S8P/C13R/D416N/R333S/R336K, S8P/C13R/D416N/R333S/R336S, S8P/C13R/D416N/R336K, S8P/C13R/D416N/R336S, or substitution corresponding thereto relative to SEQ ID NO: 1.

In embodiments, the helper enzyme comprises a deletion of about 30, or about 40, or about 50, or about 60, or about 70, or about 80, or about 90, or about 100 amino acids from an N-terminus or a C-terminus.

In embodiments, the helper enzyme comprises a deletion at positions about 1-35, or about 1-45, or about 1-55, or about 1-65, or about 1-75, or about 1-85, or about 1-95, or about 1-105, or about 2-35, or about 2-45, or about 2-68 or positions corresponding thereto, wherein the positions are relative to SEQ ID NO: 1.

In embodiments, the N terminal deletion yields reduced or ablated off-target effects of the enzyme compared to the enzyme without the N terminal deletion.

In embodiments, the helper enzyme comprising the N terminal deletion has a sequence selected from SEQ ID NO: 504, 506, or 508, or a sequence having at least about 95%, or at least about 96%, at least about 97%, at least about 98%, at least about 99% identity thereto.

In embodiments, the helper enzyme comprising an N terminal deletion is fused to a DNA binding domain, optionally wherein the DNA binding domain comprises TALEs, and/or ZnF. In embodiments, the helper enzyme further comprises a targeting element. In embodiments, the helper enzyme is suitable of inserting a donor nucleic acid comprising a transgene in a genomic safe harbor site (GSHS). In embodiments, the GSHS is in an open chromatin location in a chromosome. In embodiments, the GSHS is selected from adeno-associated virus site 1 (AAVS1), chemokine (C—C motif) receptor 5 (CCR5) gene, HIV-1 coreceptor, and human Rosa26 locus. In embodiments, the GSHS comprises one or more TTAA integration sites.

In embodiments, the targeting element directs the helper enzyme to either one or more nucleic acid binding sites that are upstream and/or downstream of the TTAA integration sites or to the TTAA integration sites. In embodiments, the targeting element directs the helper enzyme to one or more nucleic acid binding sites that are upstream and/or downstream of the TTAA integration sites and within about 5 to about 30 base pairs of the TTAA integration sites or within about 15 to about 19 base pairs of the TTAA integration sites. In embodiments, the targeting element directs the helper enzyme to two nucleic acid binding sites of the TTAA integration sites, wherein a first site is upstream of TTAA and within about 5 to about 30 base pairs or about 15 to about 19 base pairs of the TTAA and a second site is downstream of TTAA and within about 5 to about 30 base pairs or about 15 to about 19 base pairs of the TTAA.

In embodiments, the targeting element is or comprises one or more of a Cas enzyme, which is optionally catalytically inactive and which is optionally associated with a guide RNA (gRNA), a transcription activator-like effector (TALE) DNA binding domain (DBD), a Zinc finger (ZnF), a catalytically inactive transcription factor, catalytically inactive nickase, a transcriptional activator, a transcriptional repressor, a recombinase, a DNA methyltransferase, a histone methyltransferase, a paternally expressed gene 10 (PEG10), and a transposon-encoded polypeptide D (TnsD) or a variant thereof.

In embodiments, the targeting element is or comprises a TALE DBD. In embodiments, the TALE DBD comprises one or more repeat sequences. In embodiments, the TALE DBD comprises about 14, or about 15, or about, 16, or about 17, or about 18, or about 18.5 repeat sequences. In embodiments, the repeat sequences each independently comprises about 33 or 34 amino acids. In embodiments, the repeat sequences each independently comprises a repeat variable di-residue (RVD) at residue 12 or 13 of the 33 or 34 amino acids, respectively. In embodiments, the RVD recognizes one base pair in a target nucleic acid sequence.

In embodiments, the RVD recognizes a C residue in the target nucleic acid sequence and is selected from HD, N (gap), HA, ND, and HI. In embodiments, the RVD recognizes a G residue in the target nucleic acid sequence and is selected from NN, NH, NK, HN, and NA. In embodiments, the RVD recognizes an A residue in the target nucleic acid sequence and is selected from NI and NS. In embodiments, the RVD recognizes a T residue in the target nucleic acid sequence and is selected from NG, HG, H (gap), and IG.

In embodiments, the targeting element is or comprises a Cas9 enzyme associated with a gRNA or a CasX enzyme associated with a gRNA. In embodiments, the Cas9 enzyme associated with a gRNA comprises a catalytically inactive dCas9 associated with a gRNA or an inactive dCasX associated with a gRNA. In embodiments, catalytically inactive dCas9 comprises at least about 90%, or at least about 95%, or at least about 96%, or at least about 97%, or at least about 98%, or at least about 99% identity to an amino acid sequence of SEQ ID NO: 6 or a nucleic acid comprising a nucleotide sequence of SEQ ID NO: 5 or a codon-optimized form thereof.

In embodiments, the target element is or comprises a CasX enzyme associated with a gRNA, optionally wherein the catalytic inactive dCasX. In embodiments, the targeting element is or comprises a Cas12 enzyme associated with a gRNA. In embodiments, the targeting element comprises a catalytically inactive Cas12 associated with a gRNA, optionally wherein the catalytically inactive Cas12 is dCas12j or dCas12a. In embodiments, the targeting element is or comprises a TnsC, TnsB, TnsA, TniQ, Cas6, Cas7, Cas8 enzyme associated with a gRNA. In embodiments, the targeting element comprises a TniQ subdomain of TnsD.

In embodiments, the composition comprises a linker connecting the helper enzyme and the targeting element.

In embodiments, the linker comprises less than about 25 amino acids or 75 nucleotides. In embodiments, the linker comprises about 10 amino acids to about 20 amino acids or about 12 amino acids to about 15 amino acids, or about 30 nucleotides to about 60 nucleotides or about 36 nucleotides to about 45 nucleotides. In embodiments, the linker is substantially comprised of glycine (G) and serine(S) residues. In embodiments, the linker is or comprises (GSS) 4 or the linker is GS flanked on either side of a DNA binding domain, optionally TALE and ZnF. In embodiments, the linker comprises an amino acid sequence of AKLAGGAPAVGGGPKAADKFAATGGS (SEQ ID NO: 8) or a variant thereof having a substitution or deletion. In embodiments, the linker connects the targeting element to the N-terminus of the helper enzyme or connects the targeting element within the helper enzyme.

In embodiments, the composition is a nucleic acid, optionally an RNA. In embodiments, the RNA is or comprises messenger RNA (mRNA). In embodiments, the mRNA is or comprises modified mRNA (mmRNA). In embodiments, the mmRNA comprises one or more of a 5′-m7G cap (cap0, cap1, or cap2), a pseudouridine or n-methyl-pseudouridine substitution, and a poly-A tail of about 30, or of about 50, or of about 100, or of about 150 nucleotides in length.

In embodiments, the composition further comprises a donor nucleic acid or is suitable for insertion of a donor nucleic acid, optionally wherein the donor nucleic acid is a transposon. In embodiments, the helper enzyme is suitable of inserting a donor nucleic acid comprising a transgene in a genomic safe harbor site (GSHS) and/or wherein the targeting element is suitable for directing the helper enzyme to a GSHS. In embodiments, a donor DNA and a helper RNA are suitable for transfection at a donor DNA to helper RNA ratio of about 1 to about 4, or about 1 to about 2, or about 1 to about 1.

In embodiments, the composition further comprises a nucleic acid encoding a donor comprising a transgene to be integrated, optionally wherein the transgene is defective or substantially absent in a disease state. In embodiments,

In embodiments, the transgene comprises a cargo nucleic acid sequence and a first and a second donor end sequences.

In embodiments, the cargo nucleic acid sequence is flanked by the first and the second donor end sequences. In embodiments, the enzyme or variant thereof is incorporated into a vector or a vector-like particle. In embodiments, the vector or a vector-like particle comprises one or more expression cassettes. In embodiments, the vector or a vector-like particle comprises one expression cassette. In embodiments, the expression cassette further comprises the enzyme or variant thereof, the transgene, the donor end sequences, or a combination thereof. In embodiments, the enzyme or variant thereof, the transgene, the donor end sequences, or a combination thereof are incorporated into one or more vectors or vector-like particles. In embodiments, the enzyme or variant thereof, the transgene, the donor end sequences, or combination thereof are incorporated into a same vector or vector-like particle. In embodiments, the enzyme or variant thereof, the transgene, the donor end sequences, or combination thereof is incorporated into different vectors or vector-like particles. In embodiments, the vector or vector-like particle is nonviral.

In embodiments, the donor is under the control of at least one tissue-specific promoter. In embodiments, at least one tissue-specific promoter is a single promoter. In embodiments, at least one tissue-specific promoter is under the control of a dual promoter or a tandem promoter. In embodiments, the transgene to be integrated comprises at least one gene of interest. In embodiments, the transgene to be integrated comprises one gene of interest. In embodiments, the transgene to be integrated comprises two or more genes of interest. In embodiments, there is provide a host cell comprising the composition of the present disclosure. In embodiments, the composition is encapsulated in a lipid nanoparticle (LNP). In embodiments, the polynucleotide encoding the enzyme or variant thereof and the polynucleotide encoding the donor are in the form of the same LNP, optionally in a co-formulation.

In embodiments, the LNP comprises one or more lipids selected from 1,2-dioleoyl-3-trimethylammonium propane (DOTAP), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy (polyethylene glycol)-2000] (DSPE-PEG), 1,2-dimyristoyl-rac-glycero-3-methoxypolyethyleneglycol-2000 (DMG-PEG 2K), and 1,2 distearol-sn-glycerol-3phosphocholine (DSPC) and/or comprising of one or more molecules selected from polyethylenimine (PEI) and poly(lactic-co-glycolic acid) (PLGA), and N-Acetylgalactosamine (GalNAc).

In embodiments, there is provided a method for inserting a gene into the genome of a cell, comprising contacting a cell with the composition of the present disclosure. In embodiments, there is provided a method for treating a disease or disorder ex vivo, comprising contacting a cell with the composition of the present disclosure and administering the cell to a subject in need thereof. In embodiments, there is provided a method for treating a disease or disorder in vivo, comprising administering the composition of the present disclosure to a subject in need thereof.

In embodiments, there is provided a composition comprising a helper enzyme or a nucleic acid encoding the helper enzyme comprising: an amino acid sequence having at least about 80% sequence identity to SEQ ID NO: 1 and a non-polar aliphatic amino acid at position 2 of SEQ ID NO: 1, or a position corresponding thereto, and amino acid substitutions comprising S8X, C13X, R333K, K334A, N335A, R336K, and D416X of SEQ ID NO: 1, or a substitution at a position corresponding thereto.

In embodiments, there is provided a composition comprising (a) a helper enzyme or a nucleic acid encoding the helper enzyme and (b) a targeting element or a nucleic acid encoding the targeting element, wherein: the helper enzyme comprises an amino acid sequence having at least about 80% sequence identity to SEQ ID NO: 1 and has a non-polar aliphatic amino acid at position 2 of SEQ ID NO: 1 and a substitution at a position corresponding thereto, and amino acid substitutions comprising S8X, C13X, R333K, K334A, N335A, R336K, and D416X of SEQ ID NO: 1, or a substitution at a position corresponding thereto.

In embodiments, there is provided a composition comprising a helper enzyme or a nucleic acid encoding the helper enzyme comprising: an amino acid sequence having at least about 80% sequence identity to SEQ ID NO: 1 and a non-polar aliphatic amino acid at position 2 of SEQ ID NO: 1, or a position corresponding thereto, and amino acid substitutions comprising S8X, C13X, R333K, N335A, R336K, and D416X of SEQ ID NO: 1, or a substitution at a position corresponding thereto.

In embodiments, there is provided a composition comprising (a) a helper enzyme or a nucleic acid encoding the helper enzyme and (b) a targeting element or a nucleic acid encoding the targeting element, wherein: an amino acid sequence having at least about 80% sequence identity to SEQ ID NO: 1 and a non-polar aliphatic amino acid at position 2 of SEQ ID NO: 1, or a position corresponding thereto, and amino acid substitutions comprising S8X, C13X, R333K, N335A, R336K, and D416X of SEQ ID NO: 1, or a substitution at a position corresponding thereto.

In embodiments, there is provided a method of designing a transposase for binding identical target sites upstream and downstream of a target nucleic acid site, comprising: a) obtaining the transposase, the transposase being modified to have reduced or ablated inherent nucleic acid binding ability and having at least about 90%, or at least about 93%, at least about 95%, at least about 97%, at least about 98%, at least about 99% identity to the amino acid sequence of SEQ ID NO: 1, b) fusing or associating the transposase with at least two targeting elements, each targeting element being fused at or immediately after G66 relative to SEQ ID NO: 1, optionally wherein the fusing or associating the transposase with at least two targeting elements is by way of a linker, wherein the target nucleic acid site is a genomic safe harbor site of or comprising TTAA.

In embodiments, the linker is a covalent linker or a non-covalent linker. In embodiments, the linker comprises less than about 25 amino acids or 75 nucleotides. In embodiments, the linker comprises about 10 amino acids to about 20 amino acids or about 12 amino acids to about 15 amino acids, or about 30 nucleotides to about 60 nucleotides or about 36 nucleotides to about 45 nucleotides. In embodiments, the linker is substantially comprised of glycine (G) and serine(S) residues. In embodiments, the linker is or comprises (GSS)₄ or the linker is GS flanked on either side of a targeting element, optionally TALE and ZnF.

In embodiments, the linker comprises an amino acid sequence of AKLAGGAPAVGGGPKAADKFAATGGS (SEQ ID NO: 8) or a variant thereof having a substitution or deletion. In embodiments, the linker connects the targeting element to the N-terminus of the helper enzyme or connects the targeting element within the helper enzyme.

In embodiments, the linker is a non-covalent linker. In embodiments, the non-covalent linker comprises an epitope tag, optionally wherein the epitope tag comprises an ALFA tag. In embodiments, the ALFA tag further comprises recombinant variable domains of heavy-chain-only antibodies (VHH) (NANOBODY). In embodiments, the targeting elements are oriented on opposite sides of the transposase.

### BRIEF DESCRIPTION OF DRAWINGS

FIG. 1A-FIG. 1F depict six illustrative bioengineered RNA helper constructs that are contained in a replication backbone (e.g., plasmid, miniplasmid, nanoplasmid, doggybone, or close-ended linear DNA) with a T7 promoter (cap dependent), beta-globin 5′-UTR, and a helper enzyme with the S8P/C13R mutations in the Myotis lucifugus helper (relative to SEQ ID NO: 1) followed by a beta-globin 3′-UTR, and a poly-alanine tail (FIG. 1A). TALEs (FIG. 1B), ZnF (FIG. 1C), or a dead Cas (dCasX) binding proteins (FIG. 1D) with guide RNAs are joined by a covalent or non-covalent linker to the N-terminus to target the specific TTAA sites at hROSA 26, AAVS1, chromosome 4, chromosome 22, and chromosome X loci. FIG. 1E depicts a construct with a dimerization enhancer to assure activation of the two monomers. FIG. 1F depicts a construct with a DNA binder (TALE, ZnF, dCasX) that interrupts an intrinsic DNA binding loop (Y281-T291 and I332-P339) and renders the helper enzyme as Exc+/Int−.

FIG. 2A-FIG. 2B depict the non-limiting types of covalent and non-covalent linkers that are used to directly fuse (i.e., link) protein sequence-specific DNA binding domains (e.g., TALE, ZnF, Cas) that recognize target DNA sequences flanking the TTAA. In FIG. 2A, the arrow shows covalent linker that fuses DNA binders to the N-terminus of MLT transposase. In FIG. 2B, the arrows show non-covalent linkers that an antipeptide antibody (Ab) fused to a DNA binder and a peptide tag fused to the N-terminus of MLT transposase.

FIG. 3A depicts an illustrative core donor construct that is contained in a replication backbone (e.g., plasmid, miniplasmid, nanoplasmid, doggybone, or close-ended linear DNA) with a promoter driving a gene of interest (GOI) with a polyA tail flanked by two insulators and ITRs.

FIG. 3B depicts an illustrative core donor construct that is contained in a replication backbone (e.g., plasmid, miniplasmid, nanoplasmid, doggybone, or close-ended linear DNA) with a splice acceptor site for exon 2 and other exons of a gene of interest (GOI) followed by a polyA tail and flanked by ITRs.

FIG. 3C depicts an illustrative core donor construct that is contained in a replication backbone (e.g., plasmid, miniplasmid, nanoplasmid, doggybone, or close-ended linear DNA) with tandem promoters to affect expression in different tissues (e.g., without limitation, liver specific promoter, retinal specific promoter, basal lung cell promoter) and a gene(s) of interest (GOI) followed by a polyA tail and flanked by ITRs.

FIG. 3D depicts an illustrative core donor construct that is contained in a replication backbone (e.g., plasmid or miniplasmid) with two or more genes of interest (GOI) linked by P2A “self-cleaving” peptides and followed by WPRE and a polyA tail.

FIG. 3E depicts an illustrative core donor construct that is contained in a replication backbone (e.g., plasmid, miniplasmid, nanoplasmid, doggybone, or close-ended linear DNA) with a promoter(s) driving the expression of two or more genes as in FIG. 3D and linked to a sequence consisting of a 5′-miRNA, a sense and antisense miRNA pair, and completed with the 3′-miRNA.

FIG. 4A-FIG. 4C depict illustrative non-limiting concepts of bioengineering the MLT transposase protein for site-specific targeting and hetrodimerizarion. In FIG. 4A, the unengineered MLT transposase dimer binds the target DNA TTAA and flanking non-TTAA (nnnn) sequences by its natural intrinsic DNA binding domains. In FIG. 4B, the recruitment to a site-specific TTAA is directed by fusing (i.e., linking) protein sequence-specific DNA binding domains (e.g., TALE, ZnF, Cas) that recognize specific target DNA sequences flanking the TTAA. In FIG. 4C, mutations (X) in the intrinsic DNA binding domains decrease MLT transposase interactions with target DNA non-TTAA which flank the TTAA but leave excision and TTAA use intact (Exc+Int−).

FIG. 5A-FIG. 5B depict excision and integration activity of Exc+Int− candidates showing an excision-only phenotype. FIG. 5A shows excision-only candidates with integration activity below or near the negative control. FIG. 5B shows helpers with multiple mutations on a mutant background (S8P/C13R/D416N, positions relative to SEQ ID NO: 1).

FIG. 6 depict excision and integration activity of top Exc+Int− candidates fused to an E2C zinc finger DNA binding domain.

FIG. 7 depicts an illustrative 5-step plasmid landing pad assay in HEK293 cells to identify site-specific targeting using MLT transposase or other mobile elements (e.g., recombinases, integrases, transposases).

FIG. 8A depicts the landing pad and binding sites in Step 2 in FIG. 7. Landing pads with no DNA binding recognition sites are used as a negative control. A landing pad with flanking ZnF binding sites that are 15 bp from the target TTAA is used as to detect site-directed integration.

FIG. 8B depict PCR amplification to identify targeting shown in Step 4 in FIG. 7. In FIG. 8B, a 2% agarose gel shows a molecular ladder (lane 1) and the PCR products using no ZnF fused to the N-terminus of MLT S8P/C13R (Lane 2 negative control) compared to MLT with an N-terminus ZnF fusion (Lane 3) where a band at 450 bp shows targeting at the TTAA flanked by the zinc fingers as shown on FIG. 8A.

FIG. 9 depicts the results of targeting using a donor element that splices into a genomic artificial landing pad in HEK293 cells.

FIG. 10A-10D depict structure guided mutations generate MLT variants with impaired native integration activity. FIG. 10A shows a schematic for the excision assay. Seamless excision by MLT reactivates ZsGreen, quantified via flow cytometry. FIG. 10B shows a schematic for the integration assay. Genomic-integrated TurboGFP is quantified at 17 days. FIG. 10C shows an AlphaFold model of MLT. Integration-dampening mutations were made to the loop domain (I332-I338) predicted to contact the target DNA. FIG. 10D shows the effects on MLT excision and integration caused by a panel of mutations to the putative target DNA-interacting residues. Ten candidate mutants (arrows) were selected that exhibited absolute levels of >30% for excision and <5% for integration (Exc+Int−).

FIG. 11A-11E depict linking MLT to a ZnF mediates targeting to plasmids. FIG. 11A shows schematic of the plasmid-based targeting assay conducted in HEK293T cells. Targeting generated a green fluorescent signal quantified via flow cytometry. FIG. 11B shows single transposase enzyme (Ts) helper expression plasmids. Ts constructs contained internally incorporated or N-terminal DNA binding domains (DBDs) (covalent approach). A Ts-DBD pair were used in a non-covalent targeting approach where the DNA binder and Ts were linked through recombinant variable domains of heavy-chain-only antibodies (VHH) (NANOBODY)-tag association. FIG. 11C shows helpers linking E2C to MLT mediate targeting of the transposon (Tn) to reporter plasmids containing single (bars in the middle) or flanking (bars on the right) cognate e2c recognition sites near the target TTAA. Bars on the left of each condition represent reporters with no e2c recognition site. MLT was compared to the Exc+Int− mutant S8P, C13R, R333K, K334A, N335A, R336K, D416N (m1) positions relative to SEQ ID NO: 1. FIG. 11D shows PCR analysis of the plasmid-based targeting assay from FIG. 11C. Products of ~547 bp (arrow) represent insertions that occurred at or near the target erbB-2 DNA recognition site (e2c) sequence. FIG. 11E shows engineered spacing constraints for E2C MLT m1 as determined via plasmid-based targeting assay. Reporter plasmids contained equidistant e2c recognition sequences at a spacing measuring from 6-22 bp on the left (L) and right (R) of the target TTAA.

FIG. 12A-12D depict MLT targeting to endogenous sequences at octanucleotide sites. FIG. 12A shows comparison of E2C-targeted MLT variants with Exc+Int− mutations in the I332-I338 loop thought to mediate target DNA interaction. The plasmid-based targeting assay was used to quantify integration into reporters with flanking e2c recognition sites (bars on the left) or no e2c recognition sites (bars on the right) adjacent to the targeted TTAA. FIG. 12B and FIG. 12C show the taTTAAta octanucleotide was a preferential integration site for Exc+Int− mutants linked to the E2C ZnF (FIG. 12B) or E2C TALE (FIG. 12C). PCR analysis of the plasmid-based targeting assay used reporter plasmids with an octanucleotide site at an off-target (848 bp, upper arrow) or on-target position near the E2C target sequences (547 bp, lower arrow). FIG. 12D shows TALEs inserted at position G66 in MLT with Exc+Int− mutation, S8P, C13R, R333K, N335A, R336K, D416N (m2) positions relative to SEQ ID NO: 1, mediated targeting of a Tn to the human genome. TALEs were generated to nine genomic homodimer sites (HoD1-9) that each consisted of an identical 9 bp target sequence flanking a taTTAAta (HoD1-6) or ttTTAAaa (HoD7-9) octanucleotide. ddPCR was performed using a forward primer and probe in the genome and a reverse primer on either the Left or Right TIR of the Tn. All ddPCR values were normalized using the product reference ratio (PRR).

FIG. 13A-13E depict a non-covalent linkage of TALEs to MLT results in efficient targeting to the genome. FIG. 13A shows that non-covalent linkage of MLT to TALEs directs a Tn to genomic sequences incorporated in reporter plasmids in the plasmid-based targeting assay. For orientation purposes, bars on the left represent reporters with the HoD4 target site; bars on the right represent reporters with the HoD7 site. FIG. 13B shows PCR analysis of the plasmid-based targeting assay from FIG. 13A. Arrow denotes expected band size for integration product at targeted TTAA. FIG. 13C shows a non-covalent linkage strategy supported TALE directed insertion of a Tn to the genomic homodimeric site HoD4. ‘ALFA MLT m2’ was without HoD4 recombinant variable domains of heavy-chain-only antibodies (VHH) (NANOBODY) against an ALFA tag (NbALFA) and ‘ALFA MLT m2+HoD8 NbALFA’ was with a mismatched TALE. FIG. 13D shows the chromosom 6 (chr 6) heterodimeric site supported increased targeted insertion efficiency. ALFA MLT was directed by different TALEs left (ch6L NbALFA) and right (ch6R NbALFA) of the target TTAA site. ALFA MLT m2′ was without a TALE NbALFA and ‘ALFA MLT m2+HoD4 NbALFA’ was with a mismatched TALE. FIG. 13E shows the ch6 heterodimeric site supported increased targeted insertion efficiency.

FIG. 14A-14B depict excision and integration activities of MLT mutants. FIG. 14A shows alanine scanning of the predicted MLT DBD loop (I332-I338). FIG. 14B shows activities of 64 combination mutations to the MLT DBD. ‘Donor only’ control did not include a helper Ts and represents background integration.

FIG. 15A-15J shows MLT DNA spacing requirements using plasmid-based targeting assay. FIG. 15A shows PCR analysis of the plasmid-based targeting assay for different strategies linking E2C to MLT. Products of ~547 bp (black arrow) represent insertions that occurred at or near the target e2c sequence. FIG. 15B shows amplicon sequencing of products from FIG. 15A showing the percentage of reads with an insertion at a TTAA site 15 bp downstream of the target e2c sequence.

FIG. 15C shows PCR analysis comparing reporter plasmids without, one, or two e2c recognition sequences using helpers with or without the Exc+Int− mutation m1. Arrow indicates the targeted PCR product.

FIG. 15D shows amplicon sequencing of products from the bottom 2×e2c gel in FIG. 15C showing the percentage of reads at the target TTAA for the indicated reporter plasmid. The 2×e2c reporter has two e2c recognition sequences flanking the target TTAA.

FIG. 15E shows the target sequence of the 2×e2c reporter plasmid (SEQ ID NO: 882).

FIG. 15F shows PCR analysis of reporter plasmids with variable spacing of the e2c target sequences on the left (L) or right (R) of the target TTAA (6-22 bp) using the plasmid-based targeting assay. Products of ~455 bp (arrow) represent targeted insertions. A sanger sequencing chromatogram of targeted product from L15R15 (SEQ ID NO: 883) shows the junction sequence of Tn with the reporter plasmid.

FIG. 15G shows a schematic of reporter plasmids containing one or two e2c recognition sequences (arrows) that are 19 bp from the target TTAA tetranucleotide. FIG. 15H shows PCR analysis of the P2P assay using reporter plasmids with one or two e2c recognition sequences. The arrow indicates targeted product. FIG. 15I shows a comparison of CCR5-directed TALEs with matched or mismatched orientations to the target TTAA sequence using the plasmid-based targeting assay and reporter plasmids containing the CCR5 target sequences. FIG. 15J shows PCR analysis of FIG. 15I. Arrow indicates targeted product.

FIG. 16A-16C depict MLT targeting to octanucleotide sites. FIG. 16A shows mutation of the off target taTTAAta octanucleotide site in the reporter plasmid. Upper arrow indicates off-target product and lower arrow indicates targeted product. FIG. 16B shows the PCR analysis of the plasmid-based targeting assay using helpers with Exc+Int− mutations in the I332-I338 loop thought to mediate DNA interaction. Reporters did not contain off-target taTTAAta octanucleotide sites. Products of ~547 bp (black arrow) represent targeted insertions. FIG. 16C shows matched and mismatched TALE and probe combinations for helpers used to target endogenous genomic homodimeric sites (HoD1-9). Assays with mismatched TALE and probe combinations represent background integration at TTAA sites. ddPCR was performed using a forward primer and probe in the genome and a reverse primer on the Right terminal inverted repeats (TIR) of the Tn. All ddPCR values were normalized using the product reference ratio (PRR).

FIG. 17A-17E depict engineering of a non-covalent strategy linking TALEs to MLT for genome targeting. FIG. 17A shows engineered spacing constraints for a non-covalent targeting approach. The plasmid-based targeting assay was performed using reporter plasmids with e2c recognition sequences at a spacing between 16-20 bp on the left (L) and right (R) of the target TTAA. FIG. 17B shows PCR analysis of FIG. 17A. Products of ~455 bp (arrow) represent targeted insertions. FIG. 17C shows engineered position of the ALFA tag and recombinant variable domains of heavy-chain-only antibodies (VHH) (NANOBODY) (NbALFA) on helpers with and without Exc+Int− mutations (m1 and m2). ALFA MLT contains the ALFA tag at position G66. NbALFA MLT contains the ALFA tag and recombinant variable domains of heavy-chain-only antibodies (VHH) (NANOBODY) (NbALFA) at position G66. S55 ALFA MLT contains the ALFA tag at position S55. S55 NbALFA MLT contains the ALFA tag and recombinant variable domains of heavy-chain-only antibodies (VHH) (NANOBODY) (NbALFA) at position S55. FIG. 17D shows genomic targeting of the non-covalent approach using alternate Exc+Int− mutations. FIG. 17E shows a comparison of TALEs designed to bind various lengths of the chr 6 target sequence (9 bp and 12-16 bp). ALFA MLT m2 was used with a TALE ALFA tag and recombinant variable domains of heavy-chain-only antibodies (VHH) (NANOBODY) (NbALFA) designed to bind the left (L), right (R), or both sides (L+R) of the chr 6 target site. ‘ALFA MLT m2’ was missing its companion TALE NbALFA.

FIG. 18 depicts ddPCR results using the non-covalent strategy linking flanking left and right TALEs to MLT m2 for use in generating CAR-T cells expressing CD19 at the chromosome 6 genomic safe harbor site. ddPCR was performed using a forward primer and probe in the genome and a reverse primer on the right terminal inverted repeats (TIR) of the Tn. All ddPCR values were normalized using the product reference ratio (PRR). A donor without CAR19 (SEQ ID NO: 890) showed about 6.5% of cells with targeting using a single right TIR primer (i.e., haploid genome). A donor with CAR19 (SEQ ID NO: 891) showed 4% targeting using a single right TIR primer (i.e., haploid genome). For orientation purposes, the bars on the left are with TALES and bars on the right are without TALEs.

### DETAILED DESCRIPTION

In aspects there is provided a composition, and method of use of a composition, comprising a helper enzyme or a nucleic acid encoding the helper enzyme, an amino acid sequence having at least about 80% sequence identity to SEQ ID NO: 1 and a non-polar aliphatic amino acid at position 2 of SEQ ID NO: 1, or a position corresponding thereto, one or more amino acid substitutions selected from S8X, C13X, and D416X of SEQ ID NO: 1 or a substitution at a position corresponding thereto, and one or more amino acid substitutions selected from Y281X, C282X, G283X, E284X, G285X, K286X, R287X, L288X, L289X, E290X, T291X, I332X, R333X, K334X, N335X, R336X, G337X, I338X, and P339X of SEQ ID NO: 1 or a substitution at a position corresponding thereto.

In embodiments, the present disclosure provides a composition comprising a helper enzyme or a nucleic acid encoding the helper enzyme and a targeting element or a nucleic acid encoding the targeting element wherein, the helper enzyme comprises an amino acid sequence having at least about 80% sequence identity to SEQ ID NO: 1 and has a non-polar aliphatic amino acid at position 2 of SEQ ID NO: 1, or a position corresponding thereto, one or more amino acid substitutions selected from S8X, C13X, and D416X of SEQ ID NO: 1 or a substitution at a position corresponding thereto, and one or more amino acid substitutions selected from Y281X, C282X, G283X, E284X, G285X, K286X, R287X, L288X, L289X, E290X, T291X, I332X, R333X, K334X, N335X, R336X, G337X, I338X, and P339X of SEQ ID NO: 1 or a substitution at a position corresponding thereto.

In embodiments, the non-polar aliphatic amino acid is selected from alanine (A), glycine (G), valine (V), leucine (L), isoleucine (I), and proline (P).

In embodiments, X is selected from a non-polar amino acid, optionally selected from alanine (A), glycine (G), valine (V), leucine (L), isoleucine (I), proline (P), methionine (M), tryptophan (W), and phenylalanine (F), or a polar amino acid, optionally selected from serine(S), cysteine (C), asparagine (N), glutamine (Q), threonine (T), and tyrosine (Y), or a basic amino acid, optionally selected from arginine (R), histidine (H), or an acidic amino acid, optionally selected from aspartic acid (D), and glutamic acid (E), or a positively charged hydrophilic amino acid, optionally selected from lysine (K), arginine (R), and histidine (H).

In embodiments, the helper enzyme comprises a substitution at S8X of SEQ ID NO: 1 or a substitution at a position corresponding thereto, optionally wherein X is a non-polar residue, optionally selected from alanine (A), glycine (G), valine (V), leucine (L), isoleucine (I), or a polar and neutral hydrophilic residue, optionally proline (P).

In embodiments, the helper enzyme comprises a substitution at C13X of SEQ ID NO: 1 or a substitution at a position corresponding thereto, optionally wherein X is a positively charged hydrophilic amino acid, optionally selected from lysine (K), arginine (R), and histidine (H).

In embodiments, the helper enzyme comprises a substitution at D416X of SEQ ID NO: 1 or a substitution at a position corresponding thereto, wherein X is a polar and positively charged hydrophilic residue, optionally selected from arginine (R) and lysine (K), or a polar and neutral of charge hydrophilic residue, optionally selected from asparagine (N), glutamine (Q), serine(S), threonine (T), proline (P), and cysteine (C).

In embodiments, the helper enzyme comprises one, or two, or three, or four, or five, or six substitutions at positions selected from 2, 8, 13, 416, 282, 283, 284, 285, 286, 287, 288, 289, 290, 291, 332, 333, 334, 335, 336, 337, 338, and 339, or positions corresponding thereto relative to SEQ ID NO: 1.

In embodiments, the helper enzyme comprises one, or two, or three, or four substitutions at positions selected from: 2, 8, 13, 416, and one or two substitutions at positions selected from 281, 282, 283, 284, 285, 286, 287, 288, 289, 290, 291, 332, 333, 334, 335, 336, 337, 338, and 339, or positions corresponding thereto relative to SEQ ID NO: 1.

In embodiments, the helper enzyme comprises one or two, or three, or four substitutions selected from: S2A, S8P, C13R, D416A and D416N or substitutions corresponding thereto relative to SEQ ID NO: 1.

In embodiments, the help enzyme comprises a substitution selected from S8P, C13R, D416N, S8P/C13R, S8P/D416N, C13R/D416N, and S8P/C13R/D416N or substitutions corresponding thereto relative to SEQ ID NO: 1.

In embodiments, the helper enzyme comprises one or two substitutions selected from C282A, G283A, E284A, G285A, K286A, R287A, L288A, L289A, E290A, T291A, I332A, R333A, R333K, R333S, K334A, N335A, R336A, R336K, R336S, G337A, I338A, and P339A or substitutions corresponding thereto relative to SEQ ID NO: 1.

In embodiments, the composition comprises one or two, or three, or four substitutions selected from: S2A, S8P, C13R, D416A and D416N or substitutions corresponding thereto relative to SEQ ID NO: 1, and one or two substitutions selected from C282A, G283A, E284A, G285A, K286A, R287A, L288A, L289A, E290A, T291A, I332A, R333A, R333K, R333S, K334A, N335A, R336A, R336K, R336S, G337A, I338A, and P339A or substitutions corresponding thereto relative to SEQ ID NO: 1.

In embodiments, the composition one, or two, or three, or four, or five, or six substitutions selected from S2A, S8P, C13R, Y281A, C282A, G283A, E284A, G285A, K286A, R287A, L288A, L289A, E290A, T291A, I332A, R333A, R333K, R333S, K334A, N335A, R336A, R336K, R336S, G337A, I338A, P339A, D416A and D416N or substitutions corresponding thereto relative to SEQ ID NO: 1.

In embodiments, the helper enzyme comprises a substitution selected from TABLE 1, TABLE 1A, TABLE 2 and/or TABLE 2A or a substitution at positions corresponding thereto relative to SEQ ID NO: 1.

In embodiments, the helper enzyme comprises S8P/C13R/D416N/R333K/K334A/N335A/R336K, S8P/C13R/D416N/R333K/N335A/R336K, D416N/I338A, D416N/R333K, D416N/R333K/R336K, D416N/R336S, D416N/R333K/K334A, D416N/R333K/K334A/R336K, D416N/R333K/N335A, D416N/R333K/N335A/R336K, D416N/R333K/K334A/N335A, D416N/R333K/K334A/N335A/R336K, S8P/C13R/D416N/Y281A, S8P/C13R/D416N/K334A, S8P/C13R/D416N/K334A/N335A, S8P/C13R/D416N/K334A/R336A, S8P/C13R/D416N/N335A, S8P/C13R/D416N/N335A/R336A, S8P/C13R/D416N/R333A, S8P/C13R/D416N/R333A/K334A, S8P/C13R/D416N/R333A/N335A, S8P/C13R/D416N/R333A/R336A, S8P/C13R/D416N/R336A, S8P/C13R/D416N/Y281A/K334A, S8P/C13R/D416N/Y281A/K334A/N335A, S8P/C13R/D416N/K334A/N335A/R336K, S8P/C13R/D416N/K334A/N335A/R336S, S8P/C13R/D416N/K334A1R336K, S8P/C13R/D416N/K334A/R336S, S8P/C13R/D416N/N335A/R336K, S8P/C13R/D416N/N335A/R336S, S8P/C13R/D416N/R333K, S8P/C13R/D416N/R333K/K334A, S8P/C13R/D416N/R333K/K334A/N335A, S8P/C13R/D416N/R333K/K334A/N335A/R336S, S8P/C13R/D416N/R333K/K334N/R336A, S8P/C13R/D416N/R333K/K334A/R336K, S8P/C13R/D416N/R333K/K334A/R336S, S8P/C13R/D416N/R333K/N335A, S8P/C13R/D416N/R333K/N335A/R336S, S8P/C13R/D416N/R333K/R336K, S8P/C13R/D416N/R333K/R336S, S8P/C13R/D416N/R333S, S8P/C13R/D416N/R333S/K334A, S8P/C13R/D416N/R333S/K334A/N335A, S8P/C13R/D416N/R333S/K334A/N335A/R336K, S8P/C13R/D416N/R333S/K334A/N335A/R336S, S8P/C13R/D416N/R333S/K334A/R336K, S8P/C13R/D416N/R333S/K334A/R336S, S8P/C13R/D416N/R333S/N335A, S8P/C13R/D416N/R333S/N335A/R336K, S8P/C13R/D416N/R333S/N335A/R336S, S8P/C13R/D416N/R333S/R336K, S8P/C13R/D416N/R333S/R336S, S8P/C13R/D416N/R336K, S8P/C13R/D416N/R336S, or substitutions corresponding thereto relative to SEQ ID NO: 1, In embodiments, the helper enzyme comprises E2C/S8P/C13R/D416N/K334A, E2C/S8P/C13R/D416N/K334A/N335A, E2C/S8P/C13R/D416N/K334A/R336A, E2C/S8P/C13R/D416N/N335A, E2C/S8P/C13R/D416N/N335A/R336A, E2C/S8P/C13R/D416N/R333A, E2C/S8P/C13R/D416N/R333A/K334A, E2C/S8P/C13R/D416N/R333A/K335A, E2C/S8P/C13R/D416N/R333A/K336A, E2C/S8P/C13R/D416N/R336A, E2C/S8P/C13R/D416N/R281A, E2C/S8P/C13R/D416N/Y281N/K334A, E2C/S8P/C13R/D416N/Y281A/K334A/N335A, E2C/S8P/C13R/D416N/Y281N/N335A, E2C/S8P/C13R/D416N/K334A/N335A/R336K, E2C/S8P/C13R/D416N/K334A/N335A/R336S, E2C/S8P/C13R/D416N/K334A/R336K, E2C/S8P/C13R/D416N/K334A/R336S, E2C/S8P/C13R/D416N/N335A/R336K, E2C/S8P/C13R/D416N/N335A/R336S, E2C/S8P/C13R/D416N/R333K, E2C/S8P/C13R/D416N/R333K/K334A, E2C/S8P/C13R/D416N/R333K/K334A/N335A, E2C/S8P/C13R/D416N/R333K/K334A/N335A/R336K, E2C/S8P/C13R/D416N/R333K/K334A/N335N/R336S, E2C/S8P/C13R/D416N/R333K/K334N/R336A, E2C/S8P/C13R/D416N/R333K/K334A/R336K, E2C/S8P/C13R/D416N/R333K/K334A/R336S, E2C/S8P/C13R/D416N/R333K/N335A, E2C/S8P/C13R/D416N/R333K/N335A/R336K, E2C/S8P/C13R/D416N/R333K/N335A/R336S, E2C/S8P/C13R/D416N/R333K/R336K, E2C/S8P/C13R/D416N/R333K/R336S, E2C/S8P/C13R/D416N/R333S, E2C/S8P/C13R/D416N/R333S/K334A, E2C/S8P/C13R/D416N/R333S/K334A/N335A, E2C/S8P/C13R/D416N/R333S/K334A/N335A/R336K, E2C/S8P/C13R/D416N/R333S/K334A/N335A/R336S, E2C/S8P/C13R/D416N/R333S/K334A/R336K, E2C/S8P/C13R/D416N/R333S/K334A/R336S, E2C/S8P/C13R/D416N/R333S/N335A, E2C/S8P/C13R/D416N/R333S/N335A/R336K, E2C/S8P/C13R/D416N/R333S/N335A/R336S, E2C/S8P/C13R/D416N/R333S/R336K, E2C/S8P/C13R/D416N/R333S/R336S, E2C/S8P/C13R/D416N/R336K, E2C/S8P/C13R/D416N/R336S, or substitution corresponding thereto relative to SEQ ID NO: 1.

In embodiments, the helper enzyme comprises a deletion of about 30, or about 40, or about 50, or about 60, or about 70, or about 80, or about 90, or about 100 amino acids from an N-terminus or a C-terminus.

In embodiments, the helper enzyme comprises a deletion at positions about 1-35, or about 1-45, or about 1-55, or about 1-65, or about 1-75, or about 1-85, or about 1-95, or about 1-105, or about 2-35, or about 2-45, or about 2-68 or positions corresponding thereto, wherein the positions are relative to SEQ ID NO: 1.

In embodiments, the helper enzyme comprises an N-terminal deletion, optionally at amino acid positions 2-35, or 2-45, or 2-68, or positions corresponding thereto, wherein the positions are relative to SEQ ID NO: 1. In embodiments, the N-terminal deletion yields reduced or ablated off-target effects of the helper enzyme compared to the helper enzyme without the N-terminal deletion. In embodiments, the helper enzyme comprising the N-terminal deletion is or comprises an amino acid sequence of SEQ ID NO: 504, SEQ ID NO: 506, SEQ ID NO: 508 or a sequence having at least about 80%, or at least about 90%, or at least about 95%, or at least about 98% identity thereto. In embodiments, the helper enzyme comprises at least one substitution at selected from the mutations of TABLE 1, TABLE 1A, TABLE 2 and/or TABLE 2A.

In embodiments, the helper enzyme is an engineered form of an enzyme reconstructed from Myotis lucifugus. In embodiments, the helper enzyme includes but is not limited to an engineered version that is a monomer, dimer, tetramer (or another multimer), hyperactive (Exc+), and/or has a reduced interaction with non-TTAA recognitions sites (Int−), of a helper enzyme reconstructed from Myotis lucifugus or a predecessor thereof.

In embodiments, the helper enzyme has deletions which confer hyperactivity and Exc+/Int−. In some embodiments, the helper enzyme has an amino acid sequence having deletions at N-terminus positions, 2-35 (SEQ ID NO: 2), 2-45 (SEQ ID NO: 3) or 2-68 (SEQ ID NO: 3) relative to the amino acid sequence of SEQ ID NO: 1 and optionally fused to the amino acid sequence of the DNA binders in TABLES 8-17, or a functional equivalent thereof.

In embodiments, the helper enzyme further comprises a targeting element.

In embodiments, the helper enzyme is linked to a targeting element comprising one or more of a Cas enzyme, which is optionally catalytically inactive and which is optionally associated with a guide RNA (gRNA), a transcription activator-like effector (TALE) DNA binding domain (DBD), a Zinc finger (ZF) (TABLES 8-17) or a variant thereof; wherein the linker comprises less than about 25 amino acids or 75 nucleotides. In embodiments, the linker is or comprises AKLAGGAPAVGGGPKAADKFAATGGS (SEQ ID NO: 8), or a variant thereof having a substitution or deletion.

In embodiments, the linker comprises about 10 amino acids to about 20 amino acids or about 12 amino acids to about 15 amino acids, or about 30 nucleotides to about 60 nucleotides or about 36 nucleotides to about 45 nucleotides. In embodiments, there is substantially comprised of glycine (G) and serine(S) residues. In embodiments, the linker is or comprises (GSS) 4 or the linker is (GS) 1 on either side of the DNA binder (TALE, ZnF). In embodiments, the linker connects the targeting element to the N-terminus of the helper enzyme or connects the targeting element within the helper enzyme.

In embodiments, the helper enzyme is suitable of inserting a donor nucleic acid comprising a transgene in a genomic safe harbor site (GSHS) or intron 1 of a disease gene (e.g., HPRT, CFTR) and/or wherein the targeting element is suitable for directing the helper enzyme to a GSHS or intron 1 of a disease gene (e.g., HPRT, CFTR). In embodiments, the GSHS is in an open chromatin location in a chromosome. In embodiments, the GSHS is selected from the human Rosa26 locus, adeno-associated virus site 1 (AAVS1), chromosome 4 GSHS, chromosome 22 GSHS. In embodiments, the GSHS comprises one or more TTAA integration sites. In embodiments, the targeting element directs the helper enzyme to one or more nucleic acid binding sites that are upstream and/or downstream of the TTAA integration sites. In embodiments, the targeting element directs the helper enzyme to either one or more nucleic acid binding sites that are upstream and/or downstream of the TTAA integration sites or to the TTAA integration sites and within about 9 to about 30 base pairs of the TTAA integration sites or within about 15 to about 25 base pairs of the TTAA integration sites. In embodiments, the targeting element directs the helper enzyme to two nucleic acid binding sites of the TTAA integration sites, wherein a first site is upstream of TTAA and within about 9 to about 30 base pairs or about 15 to about 25 base pairs of the TTAA and a second site is downstream of TTAA and within about 9 to about 30 base pairs or about 15 to about 25 base pairs of the TTAA. In embodiments, the TTAA integration sites comprise ttTTAAaa (SEQ ID NO: 880) or taTTAAta (SEQ ID NO: 881) sites.

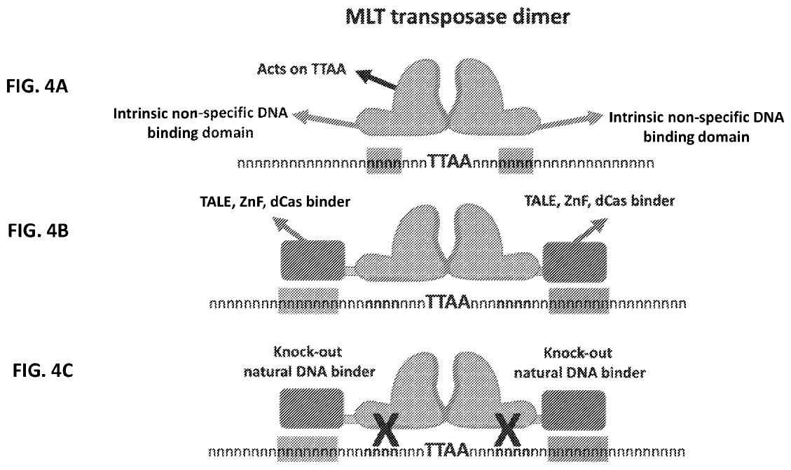

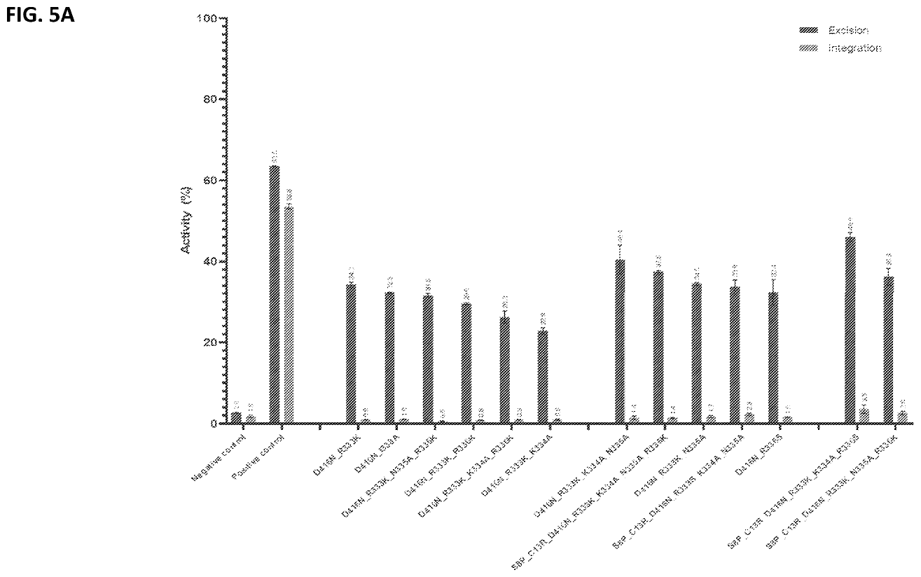

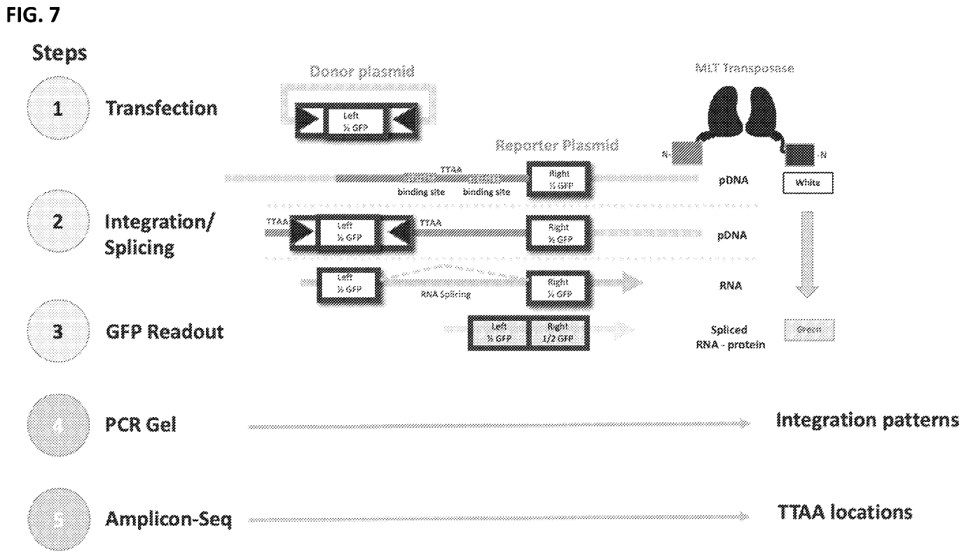

The present disclosure is based, in part, on the discovery of DNA binding proteins (e.g., without limitations, ZnF, TALE, dCas), linkers, and fusion sites that target specific TTAA integration sites (FIG. 4A-C). In embodiments, the present disclosure provides a developed landing pad assay that can show site- and sequence-specific targeting (FIG. 7). In embodiments, the landing pad assay enables Amplicon-seq to show high efficiency targeting using covalent and non-covalent linkers and flanking DNA binding recognition sites. In embodiments, the high efficiency targeting is up to about 10%, or up to about 20%, or up to about 30%, or up to about 40%, or up to about 50%, or up to about 60%, or up to about 70%, or up to about 80%, or up to about 90%, or up to about 100%. In embodiments, the flanking DNA binding recognition sites are within about 9 to about 30 base pairs of the target TTAA integration sites. In embodiments the flanking DNA binding recognition sites are within about 15 to about 25 base pairs of the target TTAA integration sites. In embodiments, the present disclosure provides MLT transposase N-terminus deletion mutants (SEQ ID NO: 504, SEQ ID NO: 506, SEQ ID NO: 508). In embodiments the MLT transposase N-terminus deletion mutants show favorable integration or epigenetic profile and promotes recruitment to intergenic target TTAA.

The present disclosure is based, in part, on the discovery of an engineered helper enzyme capable of gene insertion that finds uses in multiple applications, including, without limitation, in gene therapy. In aspects, there is provided an engineered enzyme, e.g., having an amino acid sequence of SEQ ID NO: 1 or a variant thereof, inclusive of all variants disclosed herein (e.g., SEQ ID NO: 1 and TABLES 1-2) (occasionally referred to as “engineered”, or variants thereof “MLT”, as used herein, refers to Myotis lucifugus helper, as engineered herein.

In embodiments, the illustrative bioengineered RNA helper constructs that are contained in a replication backbone (e.g., plasmid, miniplasmid, nanoplasmid, doggybone, or close-ended linear DNA) with a T7 promoter (cap dependent), beta-globin 5′-UTR, and a helper enzyme with 2 or more mutations in the Myotis lucifugus helper followed by a beta-globin 3′-UTR, and a poly-alanine tail. In embodiments, minicircle DNA is a novel, synthetic DNA vector and enzymatic DNA manufacturing process enabling rapid DNA production.

The present disclosure is based, in part, on the discovery that an enzyme capable of targeted genomic integration by transposition (e.g., an unengineered recombinase, integrase, or helper enzyme), as a monomer or a dimer, can be fused with a transcription activator-like effector proteins (TALE) DNA binding domain (DBD), a dCas/gRNA, or a zinc finger (ZnF) sequence to thereby create a chimeric enzyme capable of a site- or locus-specific transposition. For instance, in the case of a fusion to a ZnF DBD, the enzyme (e.g., without limitation, a chimeric helper) utilizes the specificity of ZnF DBD to certain sites within a host genome, which allows using DBDs to target any desired location in the genome. In this way, the chimeric helper in accordance with the present disclosure allows achieving targeted integration of a transgene.

In embodiments, the helper has one or more mutations that confer hyperactivity. In embodiments, the helper is a mammal-derived helper, optionally a helper RNA helper. Thus, the present compositions and methods for gene transfer utilize a dual donor/helper system. Transposable elements are non-viral gene delivery vehicles found ubiquitously in nature. Donor-based vectors have the capacity of stable genomic integration and long-lasting expression of transgene constructs in cells. Generally, dual donor and helper systems work via a cut-and-paste mechanism whereby donor DNA containing a transgene(s) of interest is integrated into chromosomal DNA by a helper enzyme at a repetitive sequence site. Dual donor/helper (or “donor/helper”) plasmid systems insert a transgene flanked by inverted terminal ends (“ends”), such as TTAA tetranucleotide sites, ttTTAAaa or taTTAAta octanucleotide sites, without leaving a DNA footprint in the human genome. The helper enzyme is transiently expressed (on the same or a different vector from a vector encoding the donor) and it catalyzes the insertion events from the donor plasmid to the host genome. Genomic insertions primarily target introns but may target other TTAA sites.

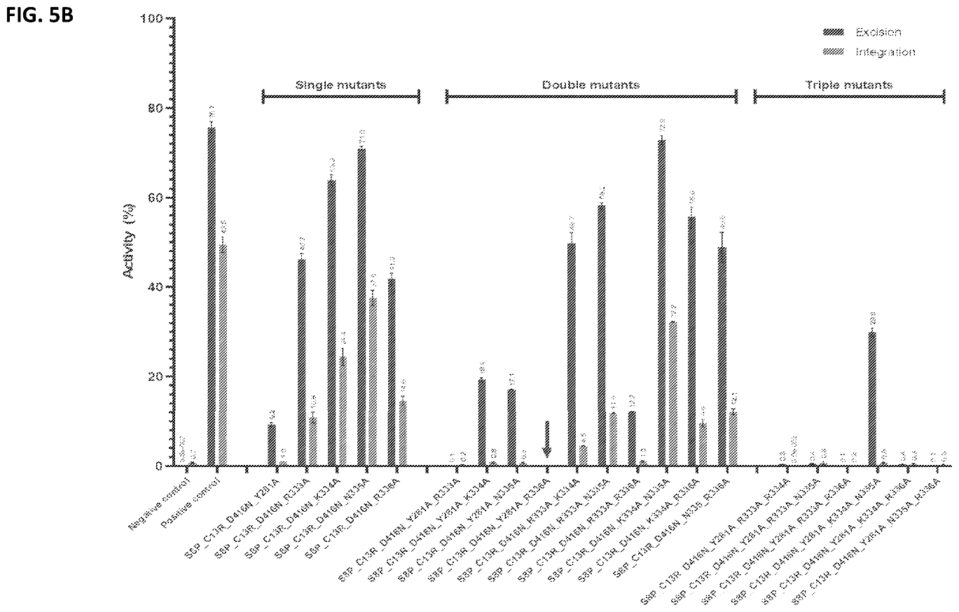

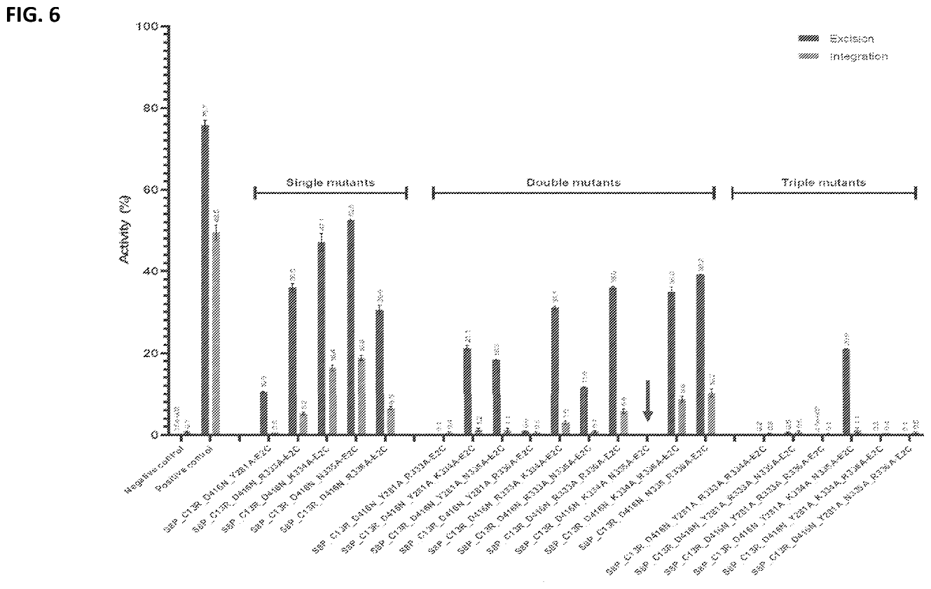

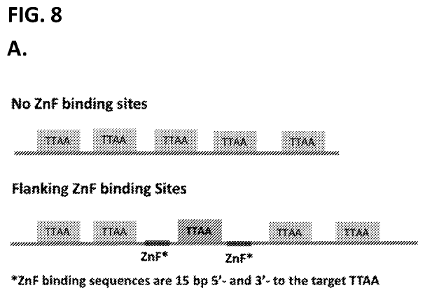

In embodiments, the disclosure describes a DNA integration system, which is highly active in mammals, and is derived from a mammalian mobile DNA element. In embodiments, this mammal-derived mobile genetic element is engineered to insert donor DNA at specific TTAA insertion “hotspots” that are frequently favored insertion sites for the un-engineered enzyme. In embodiments, this technology exploits a helper RNA encoding enzyme with engineered DNA binding proteins and a donor DNA contained between the ends of a mobile element of the gene to be inserted into the genome. In embodiments, the mammal-derived enzyme is fused to a protein domain at its N-terminus, or within internal DNA binding loops (SEQ ID NO: 1; Y281-T291 and I332-P339) (without loss of activity and “engineered” by fusing DNA binding domains (DBD) that can target almost any location in the genome). In embodiments, excision competent/target binding defective enzymes (Exc+/Int) mutants are described, that when combined with programmable, synthetic DBDs only insert at a TTAAs at a single target site (TABLE 1, TABLE 1A, TABLE 2, TABLE 2A, FIG. 5A-5B, FIG. 6, and FIG. 8A-8B). The engineered enzymes described in this disclosure displays several highly desirable features that are of great advantage for transgene integration. In embodiments, no DNA double strand breaks are introduced into the target genome. Furthermore, upon enzyme-mediated excision containing a gene of interest from its donor DNA, the flanking donor backbone ends are very efficiently rejoined, leaving no double strand break in the donor DNA to signal DNA damage. The helper enzyme inserts the excised element at high frequency selectively into a TTAA target site. Notably, because excision from the donor site results in the covalent linkage of a TTAA segment to each 5′ donor end, the joining of the 3′ donor ends to staggered positions on the top and bottom strands of the DNA flanking the target TTAA, a simple ligation restores intact duplex DNA, and no DNA synthesis is required for repair. Finally, the helper enzyme delivers a large cargo size as compared to other mobile genetic elements or integrating viral systems to date. See Liang, et al. (2009). Chromosomal mobilization and reintegration of Sleeping Beauty and PiggyBac donors. Genesis, 47(6), 404-408; Mitra, et al. (2013). Functional characterization of piggyBat from the bat Myotis lucifugus unveils an active mammalian DNA donor. Proc Natl Acad Sci USA, 110(1), 234-239; Ray, et al. (2008). Multiple waves of recent DNA donor activity in the bat, Myotis lucifugus. Genome Res, 18(5), 717-728.

In embodiments, the helper enzyme is delivered as an RNA instead of as a DNA. Other mobile genetic elements including helpers such as hyperactive piggyBac (pB) and SB100X, when delivered as RNA, have significantly less activity when compared to DNA. See Bire, et al. (2013). Exogenous mRNA delivery and bioavailability in gene transfer mediated by piggyBac transposition. BMC Biotechnol, 13, 75; Bire, et al. (2013). Optimization of the piggyBac donor using mRNA and insulators: toward a more reliable gene delivery system. PLOS One, 8(12), e82559; Wilber, et al. (2006). RNA as a source of helper for Sleeping Beauty-mediated gene insertion and expression in somatic cells and tissues. Mol Ther, 13(3), 625-630. The helper enzyme described herein has the same or better activity when delivered as RNA. The use of helper RNA offers several advantages over delivery of a DNA molecule. Wilber, et al. (2006). RNA as a source of helper for Sleeping Beauty-mediated gene insertion and expression in somatic cells and tissues. Mol Ther, 13(3), 625-630. For instance, without wishing to be bound by theory, there is improved control with respect to the duration of helper enzyme expression, minimizing persistence in the tissue, and there is potential for transgene re-mobilization and re-insertion following the initial transposition event. Furthermore, in embodiments, the helper-encoding RNA sequence is incapable of integrating into the host genome, thereby eliminating concerns about long-term helper expression and destabilizing effects with respect to the gene of interest. This safety feature, in embodiments, prevents the integration of the helper enzyme gene into the human genome and circumvents potential oncogenic and mutagenic effects.

In embodiments, the present disclosure provides a dual DNA donor and RNA helper system. The donor DNA plasmid contains helper-specific inverted terminal repeats (ITRs) flanking the transgene while the helper-RNA transiently expresses a synthetic helper enzyme that catalyzes the insertion events from the donor plasmid to the host genome.

This two component DNA/RNA system is, in embodiments, co-encapsulated in a single lipid nanoparticle using microfluidic technology and the lipid nanoparticles protect the RNA from extracellular degradation by in vivo injection.

In embodiments, the helper enzyme described herein is amenable to be fused to protein domain at the N-terminus and internal loops (Y281-T291 and I332-P339 relative to SEQ ID NO: 1) without loss of activity. Deletions of the C-terminus, in embodiments, cause a loss of helper enzyme excision and integration activity that may be restored when fused to binding ligands (e.g., rapamycin-induced FRB-FKBP fusion, SH3 plus high affinity ligand). This feature permits, inter alia, the synthesis of an “engineered” helper enzyme that target specific genomic regions of interest by fusing to the helper enzyme particular DNA binding domains that can target almost any location in the genome.

### Helper Enzyme

In embodiments, the present disclosure provides a composition comprising a helper enzyme or a nucleic acid encoding the helper enzyme, an amino acid sequence having at least about 80% sequence identity to SEQ ID NO: 1 and a non-polar aliphatic amino acid at position 2 of SEQ ID NO: 1, or a position corresponding thereto, one or more amino acid substitutions selected from S8X, C13X, and D416X of SEQ ID NO: 1 or a substitution at a position corresponding thereto, and one or more amino acid substitutions selected from Y281X, C282X, G283X, E284X, G285X, K286X, R287X, L288X, L289X, E290X, T291X, I332X, R333X, K334X, N335X, R336X, G337X, I338X, and P339X of SEQ ID NO: 1 or a substitution at a position corresponding thereto.

In embodiments, the present disclosure provides a composition comprising a helper enzyme or a nucleic acid encoding the helper enzyme and a targeting element or a nucleic acid encoding the targeting element wherein, the helper enzyme comprises an amino acid sequence having at least about 80% sequence identity to SEQ ID NO: 1 and has a non-polar aliphatic amino acid at position 2 of SEQ ID NO: 1, or a position corresponding thereto, one or more amino acid substitutions selected from S8X, C13X, and D416X of SEQ ID NO: 1 or a substitution at a position corresponding thereto, and one or more amino acid substitutions selected from Y281X, C282X, G283X, E284X, G285X, K286X, R287X, L288X, L289X, E290X, T291X, I332X, R333X, K334X, N335X, R336X, G337X, I338X, and P339X of SEQ ID NO: 1 or a substitution at a position corresponding thereto.

SEQ ID NO: 1: amino acid sequence helper transposase. Without
wishing to be bound by a particular theory, D416N rescues excision
deficiency. In embodiments, the bolded amino acids in the DNA
binding loop (Y281-T291 and 1332-P339) and underlined
amino acids were mutated. (572 amino acids)

| | | | | | | |
|---|---|---|---|---|---|---|
| 1 | MAQHSDYSDD | EFCADKLSNY | SCDSDLENAS | TSDEDSSDDE | VMVRPRTLRR | RRISSSSSDS |
| 61 | ESDIEGGREE | WSHVDNPPVL | EDELGHOGLN | TDAVINNIED | AVKLFIGDDF | FEFLVEESNR |
| 121 | YYNQNRNNFK | LSKKSLKWKD | ITPQEMKKEL | GLIVLMGQVR | KDRRDDYWTT | EPWTETPYFG |
| 181 | KTMTRDRERQ | IWKAWHENNN | ADIVNESDRL | CKVRPVLDYF | VPKFINIYKP | HQQLSLDEGI |
| 241 | VPWRGRLFFR | VYNAGKIVKY | GILVRLLCES | DTGYICNMEI | YCGEGKRLLE | TIQTVVSPYT |
| 301 | DSWYHIYMDN | YYNSVANCEA | LMKNKFRICG | TIRKNRGIPK | DFQTISLKKG | ETKFIRKNDI |
| 361 | LLQVWQSKKP | VYLISSIHSA | EMEESQNIDR | TSKKKIVKPN | ALIDYNKHMK | GVDRADQYLS |
| 421 | YYSILRRTVK | WTKRLAMYMI | NCALENSYAV | YKSVRQRKMG | FKMELKQTAI | HWLTDDIPED |
| 481 | MDIVPDLQPV | PSTSGMRAKP | PTSDPPCRLS | MDMRKHTLQA | IVGSGKKKNI | LRRCRVCSVH |
| 541 | KLRSETRYMC | KFCNIPLHKG | ACFEKYHTLK | NY | | |

In embodiments, the helper enzyme comprises an amino acid sequence of at least about 90% identity to SEQ ID NO: 1. In embodiments, the helper enzyme comprises an amino acid sequence of at least about 93% identity to SEQ ID NO: 1. In embodiments, the helper enzyme comprises an amino acid sequence of at least about 95% identity to SEQ ID NO: 1. In embodiments, the helper enzyme comprises an amino acid sequence of at least about 98% identity to SEQ ID NO: 1. In embodiments, the helper enzyme comprises an amino acid sequence of at least about 99% identity to SEQ ID NO: 1.

SEQ ID NO: 10: nucleotide sequence encoding SEQ ID NO: 1 (1719 nt)

| | | | | | | |
|---|---|---|---|---|---|---|
| 1 | ATGGCCCAGC | ACAGCGACTA | CCCCGACGAC | GAGTTCAGAG | CCGATAAGCT | GAGTAACTAC |
| 61 | AGCTGCGACA | GCGACCTGGA | AAACGCCAGC | ACATCCGACG | AGGACAGCTC | TGACGACGAG |
| 121 | GTGATGGTGC | GGCCCAGAAC | CCTGAGACGG | AGAAGAATCA | GCAGCTCTAG | CAGCGACTCT |
| 181 | GAATCCGACA | TCGAGGGCGG | CCGGGAAGAG | TGGAGCCACG | TGGACAACCC | TCCTGTTCTG |
| 241 | GAAGATTTTC | TGGGCCATCA | GGGCCTGAAC | ACCGACGCCG | TGATCAACAA | CATCGAGGAT |
| 301 | GCCGTGAAGC | TGTTCATAGG | AGATGATTTC | TTTGAGTTCC | TGGTCGAGGA | ATCCAACCGC |
| 361 | TATTACAACC | AGAATAGAAA | CAACTTCAAG | CTGAGCAAGA | AAAGCCTGAA | GTGGAAGGAC |
| 421 | ATCACCCCTC | AGGAGATGAA | AAAGTTCCTG | GGACTGATCG | TTCTGATGGG | ACAGGTGCGG |
| 481 | AAGGACAGAA | GGGATGATTA | CTGGACAACC | GAACCTTGGA | CCGAGACCCC | TTACTTTGGC |
| 541 | AAGACCATGA | CCAGAGACAG | ATTCAGACAG | ATCTGGAAAG | CCTGGCACTT | CAACAACAAT |
| 601 | GCTGATATCG | TGAACGAGTC | TGATAGACTG | TGTAAAGTGC | GGCCAGTGTT | GGATTACTTC |
| 661 | GTGCCTAAGT | TCATCAACAT | CTATAAGCCT | CACCAGCAGC | TGAGCCTGGA | TGAAGGCATC |
| 721 | GTGCCCTGGC | GGGGCAGACT | GTTCTTCAGA | GTGTACAATG | CTGGCAAGAT | CGTCAAATAC |
| 781 | GGCATCCTGG | TGCGCCTTCT | GTGCGAGAGC | GATACAGGCT | ACATCTGTAA | TATGGAAATC |
| 841 | TACTGCGGCG | AGGGCAAAAG | ACTGCTGGAA | ACCATCCAGA | CCGTCGTTTC | CCCTTATACC |
| 901 | GACAGCTGGT | ACCACATCTA | CATGGACAAC | TACTACAATT | CTGTGGCCAA | CTGCGAGGCC |
| 961 | CTGATGAAGA | ACAAGTTTAG | AATCTGCGGC | ACAATCAGAA | AAAACAGAGG | CATCCCTAAG |
| 1021 | GACTTCCAGA | CCATCTCTCT | GAAGAAGGGC | GAAACCAAGT | TCATCAGAAA | GAACGACATC |
| 1081 | CTGCTCCAAG | TGTGGCAGTC | CAAGAAACCC | GTGTACCTGA | TCAGCAGCAT | CCATAGCGCC |
| 1141 | GAGATGGAAG | AAAGCCAGAA | CATCGACAGA | ACAAGCAAGA | AGAAGATCGT | GAAGCCCAAT |
| 1201 | GCTCTGATCG | ACTACAACAA | GCACATGAAA | GGCGTGGACC | GGGCCGACCA | GTACCTGTCT |
| 1261 | TATTACTCTA | TCCTGAGAAG | AACAGTGAAA | TGGACCAAGA | GACTGGCCAT | GTACATGATC |
| 1321 | AATTGCGCCC | TGTTCAACAG | CTACGCCGTG | TACAAGTCCG | TGCGACAAAG | AAAAATGGGA |
| 1381 | TTCAAGATGT | TCCTGAAGCA | GACAGCCATC | CACTGGCTGA | CAGACGACAT | TCCTGAGGAC |
| 1441 | ATGGACATTG | TGCCAGATCT | GCAACCTGTG | CCCAGCACCT | CTGGTATGAG | AGCTAAGCCT |
| 1501 | CCCACCAGCG | ATCCTCCATG | TAGACTGAGC | ATGGACATGC | GGAAGCACAC | CCTGCAGGCC |
| 1561 | ATCGTCGGCA | GCGGCAAGAA | GAAGAACATC | CTTAGACGGT | GCAGGGTGTG | CAGCGTGCAC |
| 1621 | AAGCTGCGGA | GCGAGACTCG | GTACATGTGC | AAGTTTTGCA | ACATTCCCCT | GCACAAGGGA |
| 1681 | GCCTGCTTCG | AGAAGTACCA | CACCCTGAAG | AATTACTAG | | |

In embodiments, the nucleic acid that encodes the helper enzyme has a nucleotide sequence of SEQ ID NO: 10 or a codon-optimized form thereof.

```
SEQ ID NO: 2: amino acid sequence of a variant of a hyperactive
helper with P at position 8 and R at position 13
(S8P/C13R)(572 amino acids)
    1 MAQHSDYPDD EFRADKLSNY SCDSDLENAS TSDEDSSDDE VMVRPRTLRR RRISSSSSDS
  61 ESDIEGGREE WSHVDNPPVL EDFLGHQGLN TDAVINNIED AVKLFIGDDF FEFLVEESNR
121 YYNQNRNNFK LSKKSLKWKD ITPQEMKKEL GLIVLMGQVR KDRRDDYWTT EPWTETPYFG
181 KTMTRDRERQ IWKAWHENNN ADIVNESDRL CKVRPVLDYF VPKFINIYKP HQQLSLDEGI
241 VPWRGRLFFR VYNAGKIVKY GILVRLLCES DTGYICNMEI YCGEGKRLLE TIQTVVSPYT
301 DSWYHIYMDN YYNSVANCEA LMKNKFRICG TIRKNRGIPK DFQTISLKKG ETKFIRKNDI
361 LLQVWQSKKP VYLISSIHSA EMEESQNIDR TSKKKIVKPN ALIDYNKHMK GVDRADQYLS
421 YYSILRRTVK WTKRLAMYMI NCALENSYAV YKSVRQRKMG FKMELKQTAI HWLTDDIPED
481 MDIVPDLQPV PSTSGMRAKP PTSDPPCRLS MDMRKHTLQA IVGSGKKKNI LRRCRVCSVH
541 KLRSETRYMC KFCNIPLHKG ACFEKYHTLK NY
```

In embodiments, the helper enzyme comprises an amino acid sequence of at least about 90% identity to SEQ ID NO: 2. In embodiments, the helper enzyme comprises an amino acid sequence of at least about 93% identity to SEQ ID NO: 2. In embodiments, the helper enzyme comprises an amino acid sequence of at least about 95% identity to SEQ ID NO: 2. In embodiments, the helper enzyme comprises an amino acid sequence of at least about 98% identity to SEQ ID NO: 2. In embodiments, the helper enzyme comprises an amino acid sequence of at least about 99% identity to SEQ ID NO: 2.

| nucleotide sequence encoding a variant of a hyperactive | |
|---|---|
| helper with P at position 8 and R at position | |
| 13 (S8P/C13R)(1716 nt) | |
| SEQ ID NO: 7 | |
| 1 | ATGGCCCAGC ACAGCGACTA CCCCGACGAC GAGTTCAGAG CCGATAAGCT GAGTAACTAC |
| 61 | AGCTGCGACA GCGACCTGGA AAACGCCAGC ACATCCGACG AGGACAGCTC TGACGACGAG |
| 121 | GTGATGGTGC GGCCCAGAAC CCTGAGACGG AGAAGAATCA GCAGCTCTAG CAGCGACTCT |
| 181 | GAATCCGACA TCGAGGGCGG CCGGGAAGAG TGGAGCCACG TGGACAACCC TCCTGTTCTG |
| 241 | GAAGATTTTC TGGGCCATCA GGGCCTGAAC ACCGACGCCG TGATCAACAA CATCGAGGAT |
| 301 | GCCGTGAAGC TGTTCATAGG AGATGATTTC TTTGAGTTCC TGGTCGAGGA ATCCAACCGC |
| 361 | TATTACAACC AGAATAGAAA CAACTTCAAG CTGAGCAAGA AAAGCCTGAA GTGGAAGGAC |
| 421 | ATCACCCCTC AGGAGATGAA AAAGTTCCTG GGACTGATCG TTCTGATGGG ACAGGTGCGG |
| 481 | AAGGACAGAA GGGATGATTA CTGGACAACC GAACCTTGGA CCGAGACCCC TTACTTTGGC |
| 541 | AAGACCATGA CCAGAGACAG ATTCAGACAG ATCTGGAAAG CCTGGCACTT CAACAACAAT |
| 601 | GCTGATATCG TGAACGAGTC TGATAGACTG TGTAAAGTGC GGCCAGTGTT GGATTACTTC |
| 661 | GTGCCTAAGT TCATCAACAT CTATAAGCCT CACCAGCAGC TGAGCCTGGA TGAAGGCATC |
| 721 | GTGCCCTGGC GGGGCAGACT GTTCTTCAGA GTGTACAATG CIGGCAAGAT CGTCAAATAC |
| 781 | GGCATCCTGG TGCGCCTTCT GTGCGAGAGC GATACAGGCT ACATCTGTAA TATGGAAATC |
| 841 | TACTGCGGCG AGGGCAAAAG ACTGCTGGAA ACCATCCAGA CCGTCGTTTC CCCTTATACC |
| 901 | GACAGCTGGT ACCACATCTA CATGGACAAC TACTACAATT CTGTGGCCAA CTGCGAGGCC |
| 961 | CTGATGAAGA ACAAGTTTAG AATCTGCGGC ACAATCAGAA AAAACAGAGG CATCCCTAAG |
| 1021 | GACTTCCAGA CCATCTCTCT GAAGAAGGGC GAAACCAAGT TCATCAGAAA GAACGACATC |
| 1081 | CTGCTCCAAG TGTGGCAGTC CAAGAAACCC GTGTACCTGA TCAGCAGCAT CCATAGCGCC |
| 1141 | GAGATGGAAG AAAGCCAGAA CATCGACAGA ACAAGCAAGA AGAAGATCGT GAAGCCCAAT |
| 1201 | GCTCTGATCG ACTACAACAA GCACATGAAA GGCGTGGACC GGGCCGACCA GTACCTGTCT |
| 1261 | TATTACTCTA TCCTGAGAAG AACAGTGAAA TGGACCAAGA GACTGGCCAT GTACATGATC |
| 1321 | AATTGCGCCC TGTTCAACAG CTACGCCGTG TACAAGTCCG TGCGACAAAG AAAAATGGGA |
| 1381 | TTCAAGATGT TCCTGAAGCA GACAGCCATC CACIGGCTGA CAGACGACAT TCCTGAGGAC |
| 1441 | ATGGACATTG TGCCAGATCT GCAACCTGTG CCCAGCACCT CTGGTATGAG AGCTAAGCCT |
| 1501 | CCCACCAGCG ATCCTCCATG TAGACTGAGC ATGGACATGC GGAAGCACAC CCTGCAGGCC |
| 1561 | ATCGTCGGCA GCGGCAAGAA GAAGAACATC CTTAGACGGT GCAGGGTGTG CAGCGTGCAC |
| 1621 | AAGCTGCGGA GCGAGACTCG GTACATGTGC AAGTTTTGCA ACATTCCCCT GCACAAGGGA |
| 1681 | GCCTGCTTCG AGAAGTACCA CACCCTGAAG AATTAC |

In embodiments, the helper enzyme has one or more mutations which confer hyperactivity.

In embodiments, the nucleic acid that encodes the helper enzyme has a nucleotide sequence of SEQ ID NO: 7 or a codon-optimized form thereof.

In embodiments, the helper enzyme has one or more amino acid substitutions selected from S8X, C13X, and/or D416X of SEQ ID NO: 1 or a substitution at a position corresponding thereto.

In embodiments, the helper enzyme comprises a substitution at S8X of SEQ ID NO: 1 or a substitution at a position corresponding thereto, optionally wherein X is a non-polar residue, optionally selected from alanine (A), glycine (G), valine (V), leucine (L), isoleucine (I), or a polar and neutral hydrophilic residue, optionally proline (P).

In embodiments, the helper enzyme comprises a substitution at C13X of SEQ ID NO: 1 or a substitution at a position corresponding thereto, optionally wherein X is a positively charged hydrophilic amino acid, optionally selected from lysine (K), arginine (R), and histidine (H).

In embodiments, the helper enzyme comprises a substitution at D416X of SEQ ID NO: 1 or a substitution at a position corresponding thereto, wherein X is a polar and positively charged hydrophilic residue, optionally selected from arginine (R) and lysine (K), or a polar and neutral of charge hydrophilic residue, optionally selected from asparagine (N), glutamine (Q), serine(S), threonine (T), proline (P), and cysteine (C).

In embodiments, the helper enzyme comprises one, or two, or three, or four, or five, or six substitutions at positions selected from 2, 8, 13, 416, 282, 283, 284, 285, 286, 287, 288, 289, 290, 291, 332, 333, 334, 335, 336, 337, 338, and 339, or positions corresponding thereto relative to SEQ ID NO: 1.

In embodiments, the helper enzyme comprises one, or two, or three, or four substitutions at positions selected from: 2, 8, 13, 416, and one or two substitutions at positions selected from 281, 282, 283, 284, 285, 286, 287, 288, 289, 290, 291, 332, 333, 334, 335, 336, 337, 338, and 339, or positions corresponding thereto relative to SEQ ID NO: 1.

In embodiments, the helper enzyme comprises one or two, or three, or four substitutions selected from: S2A, S8P, C13R, D416A and D416N or substitutions corresponding thereto relative to SEQ ID NO: 1.

In embodiments, the help enzyme comprises a substitution selected from S8P, C13R, D416N, S8P/C13R, S8P/D416N, C13R/D416N, and S8P/C13R/D416N or substitutions corresponding thereto relative to SEQ ID NO: 1.

In embodiments, the helper enzyme comprises one or two substitutions selected from C282A, G283A, E284A, G285A, K286A, R287A, L288A, L289A, E290A, T291A, I332A, R333A, R333K, R333S, K334A, N335A, R336A, R336K, R336S, G337A, I338A, and P339A or substitutions corresponding thereto relative to SEQ ID NO: 1.

In embodiments, the composition comprises one or two, or three, or four substitutions selected from: S2A, S8P, C13R, D416A and D416N or substitutions corresponding thereto relative to SEQ ID NO: 1, and one or two substitutions selected from C282A, G283A, E284A, G285A, K286A, R287A, L288A, L289A, E290A, T291A, I332A, R333A, R333K, R333S, K334A, N335A, R336A, R336K, R336S, G337A, I338A, and P339A or substitutions corresponding thereto relative to SEQ ID NO: 1.

In embodiments, the composition one, or two, or three, or four, or five, or six substitutions selected from S2A, S8P, C13R, Y281A, C282A, G283A, E284A, G285A, K286A, R287A, L288A, L289A, E290A, T291A, I332A, R333A, R333K, R333S, K334A, N335A, R336A, R336K, R336S, G337A, I338A, P339A, D416A and D416N or substitutions corresponding thereto relative to SEQ ID NO: 1.

In embodiments, the helper enzyme comprises a substitution selected from TABLE 1, TABLE 1A, TABLE 2 and/or TABLE 2A or a substitution at positions corresponding thereto relative to SEQ ID NO: 1.

In embodiments, the helper enzyme comprises at least one substitution at positions selected from: 2, 8, 13, 125, 281, 282, 283, 284, 285, 286, 287, 288, 289, 290, 291, 332, 333, 334, 335, 336, 337, 338, 339, and 416, or positions corresponding thereto relative to SEQ ID NO: 1.

In embodiments, the helper enzyme comprises S8P/C13R/D416N/R333K/K334A/N335A/R336K, S8P/C13R/D416N/R333K/N335A/R336K, D416N/I338A, D416N/R333K, D416N/R333K/R336K, D416N/R336S, D416N/R333K/K334A, D416N/R333K/K334A/R336K, D416N/R333K/N335A, D416N/R333K/N335A/R336K, D416N/R333K/K334A/N335A, D416N/R333K/K334A/N335A/R336K, S8P/C13R/D416N/Y281A, S8P/C13R/D416N/K334A, S8P/C13R/D416N/K334A/N335A, S8P/C13R/D416N/K334A/R336A, S8P/C13R/D416N/N335A, S8P/C13R/D416N/N335A/R336A, S8P/C13R/D416N/R333A, S8P/C13R/D416N/R333A/K334A, S8P/C13R/D416N/R333A/N335A, S8P/C13R/D416N/R333A/R336A, S8P/C13R/D416N/R336A, S8P/C13R/D416N/Y281A/K334A, S8P/C13R/D416N/Y281A/K334A/N335A, S8P/C13R/D416N/K334A/N335A/R336K, S8P/C13R/D416N/K334A/N335A/R336S, S8P/C13R/D416N/K334A/R336K, S8P/C13R/D416N/K334A/R336S, S8P/C13R/D416N/N335A/R336K, S8P/C13R/D416N/N335A/R336S, S8P/C13R/D416N/R333K, S8P/C13R/D416N/R333K/K334A, S8P/C13R/D416N/R333K/K334A/N335A, S8P/C13R/D416N/R333K/K334A/N335A/R336S, S8P/C13R/D416N/R333K/K334A/R336A, S8P/C13R/D416N/R333K/K334A/R336K, S8P/C13R/D416N/R333K/K334A/R336S, S8P/C13R/D416N/R333K/N335A, S8P/C13R/D416N/R333K/N335A/R336S, S8P/C13R/D416N/R333K/R336K, S8P/C13R/D416N/R333K/R336S, S8P/C13R/D416N/R333S, S8P/C13R/D416N/R333S/K334A, S8P/C13R/D416N/R333S/K334A/N335A, S8P/C13R/D416N/R333S/K334A/N335A/R336K, S8P/C13R/D416N/R333S/K334A/N335A/R336S, S8P/C13R/D416N/R333S/K334A/R336K, S8P/C13R/D416N/R333S/K334A/R336S, S8P/C13R/D416N/R333S/N335A, S8P/C13R/D416N/R333S/N335A/R336K, S8P/C13R/D416N/R333S/N335A/R336S, S8P/C13R/D416N/R333S/R336K, S8P/C13R/D416N/R333S/R336S, S8P/C13R/D416N/R336K, S8P/C13R/D416N/R336S, or substitutions corresponding thereto relative to SEQ ID NO: 1, In embodiments, the helper enzyme comprises E2C/S8P/C13R/D416N/K334A, E2C/S8P/C13R/D416N/K334A/N335A, E2C/S8P/C13R/D416N/K334A/R336A, E2C/S8P/C13R/D416N/N335A, E2C/S8P/C13R/D416N/N335A/R336A, E2C/S8P/C13R/D416N/R333A, E2C/S8P/C13R/D416N/R333A/K334A, E2C/S8P/C13R/D416N/R333A/K335A, E2C/S8P/C13R/D416N/R333A/K336A, E2C/S8P/C13R/D416N/R336A, E2C/S8P/C13R/D416N/R281A, E2C/S8P/C13R/D416N/Y281A/K334A, E2C/S8P/C13R/D416N/Y281A/K334A/N335A, E2C/S8P/C13R/D416N/Y281A/N335A, E2C/S8P/C13R/D416N/K334A/N335A/R336K, E2C/S8P/C13R/D416N/K334A/N335A/R336S, E2C/S8P/C13R/D416N/K334A/R336K, E2C/S8P/C13R/D416N/K334A/R336S, E2C/S8P/C13R/D416N/N335A/R336K, E2C/S8P/C13R/D416N/N335A/R336S, E2C/S8P/C13R/D416N/R333K, E2C/S8P/C13R/D416N/R333K/K334A, E2C/S8P/C13R/D416N/R333K/K334A/N335A, E2C/S8P/C13R/D416N/R333K/K334A/N335A/R336K, E2C/S8P/C13R/D416N/R333K/K334A/N335A/R336S, E2C/S8P/C13R/D416N/R333K/K334A/R336A, E2C/S8P/C13R/D416N/R333K/K334A/R336K, E2C/S8P/C13R/D416N/R333K/K334A/R336S, E2C/S8P/C13R/D416N/R333K/N335A, E2C/S8P/C13R/D416N/R333K/N335A/R336K, E2C/S8P/C13R/D416N/R333K/N335A/R336S, E2C/S8P/C13R/D416N/R333K/R336K, E2C/S8P/C13R/D416N/R333K/R336S, E2C/S8P/C13R/D416N/R333S, E2C/S8P/C13R/D416N/R333S/K334A, E2C/S8P/C13R/D416N/R333S/K334A/N335A, E2C/S8P/C13R/D416N/R333S/K334A/N335A/R336K, E2C/S8P/C13R/D416N/R333S/K334A/N335A/R336S, E2C/S8P/C13R/D416N/R333S/K334A/R336K, E2C/S8P/C13R/D416N/R333S/K334A/R336S, E2C/S8P/C13R/D416N/R333S/N335A, E2C/S8P/C13R/D416N/R333S/N335A/R336K, E2C/S8P/C13R/D416N/R333S/N335A/R336S, E2C/S8P/C13R/D416N/R333S/R336K, E2C/S8P/C13R/D416N/R333S/R336S, E2C/S8P/C13R/D416N/R336K, E2C/S8P/C13R/D416N/R336S, or substitution corresponding thereto relative to SEQ ID NO: 1.

TABLE 1
Transposase helper mutations screened for Exc + Int-activity,
mutations relative to SEQ ID NO: 1

| ID | Mutations |
|---|---|
| DSGT-0648 | S8P_C13R_C282A |
| DSGT-0649 | S8P_C13R_L288A |
| DSGT-0650 | S8P_C13R_L289A |
| DSGT-0651 | S8P_C13R_E290A |
| DSGT-0652 | S8P_C13R_T291A |
| DSGT-0653 | S8P_C13R_P339A |
| DSGT-0654 | S8P_C13R_R333K |
| DSGT-0655 | S8P_C13R_R333S |
| DSGT-0656 | S8P_C13R_R333K_R336K |
| DSGT-0657 | S8P_C13R_R333S_R336K |
| DSGT-0658 | S8P_C13R_R333K_R336S |
| DSGT-0659 | S8P_C13R_R333S_R336S |
| DSGT-0660 | S8P_C13R_R336K |
| DSGT-0661 | S8P_C13R_R336S |
| DSGT-0662 | S8P_C13R_R333K_K334A |
| DSGT-0663 | S8P_C13R_R333S_K334A |
| DSGT-0664 | S8P_C13R_R333K_K334A_R336K |
| DSGT-0665 | S8P_C13R_R333S_K334A_R336K |
| SGT-0666 | S8P_C13R_R333K_K334A_R336S |
| DSGT-0667 | S8P_C13R_R333S_K334A_R336S |
| DSGT-0668 | S8P_C13R_K334A_R336K |
| DSGT-0669 | S8P_C13R_K334A_R336S |
| DSGT-0670 | S8P_C13R_R333K_N335A |
| DSGT-0671 | S8P_C13R_R333S_N335A |
| DSGT-0672 | S8P_C13R_R333K_N335A_R336K |
| DSGT-0673 | S8P_C13R_R333S_N335A_R336K |
| DSGT-0674 | S8P_C13R_R333K_N335A_R336S |
| DSGT-0675 | S8P_C13R_R333S_N335A_R336S |
| DSGT-0676 | S8P_C13R_N335A_R336K |
| DSGT-0677 | S8P_C13R_N335A_R336S |
| DSGT-0678 | S8P_C13R_R333K_K334A_N335A |
| DSGT-0679 | S8P_C13R_R333S_K334A_N335A |
| DSGT-0680 | S8P_C13R_R333K_K334A_N335A_R336K |
| DSGT-0681 | S8P_C13R_R333S_K334A_N335A_R336K |
| DSGT-0682 | S8P_C13R_R333K_K334A_N335A_R336S |
| DSGT-0683 | S8P_C13R_R333S_K334A_N335A_R336S |
| DSGT-0684 | S8P_C13R_K334A_N335A_R336K |
| DSGT-0685 | S8P_C13R_K334A_N335A_R336S |
| DSGT-0686 | S8P_C13R_D416N_G285A_K286A |
| DSGT-0687 | S8P_C13R_D416N_G285A_R287A |
| DSGT-0688 | S8P_C13R_D416N_G285A_1332A |
| DSGT-0689 | S8P_C13R_D416N_G285A_R333A |
| DSGT-0690 | S8P_C13R_D416N_G285A_N335A |
| DSGT-0691 | S8P_C13R_D416N_G285A_R336A |
| DSGT-0692 | S8P_C13R_D416N_G285A_G337A |
| DSGT-0693 | S8P_C13R_D416N_G285A_I338A |
| DSGT-0694 | S8P_C13R_D416N_K286A_R287A |
| DSGT-0695 | S8P_C13R_D416N_K286A_1332A |
| DSGT-0696 | S8P_C13R_D416N_K286A_R333A |
| DSGT-0697 | S8P_C13R_D416N_K286A_N335A |
| DSGT-0698 | S8P_C13R_D416N_K286A_R336A |
| DSGT-0699 | S8P_C13R_D416N_K286A_G337A |
| DSGT-0700 | S8P_C13R_D416N_K286A_I338A |
| DSGT-0701 | S8P_C13R_D416N_R287A_1332A |
| DSGT-0702 | S8P_C13R_D416N_R287A_N335A |
| DSGT-0703 | S8P_C13R_D416N_R287A_R336A |
| DSGT-0704 | S8P_C13R_D416N_R287A_G337A |
| DSGT-0705 | S8P_C13R_D416N_R287A_I338A |
| DSGT-0706 | S8P_C13R_D416N_1332A_N335A |
| DSGT-0707 | S8P_C13R_D416N_1332A_R336A |
| DSGT-0708 | S8P_C13R_D416N_1332A_G337A |
| DSGT-0709 | S8P_C13R_D416N_1332A_I338A |
| DSGT-0710 | S8P_C13R_D416N_R287A_R333A |
| DSGT-0711 | S8P_C13R_D416N_1332A_R333A |
| DSGT-0712 | S8P_C13R_D416N_R333A_N335A |
| DSGT-0713 | S8P_C13R_D416N_R333A_R336A |
| DSGT-0714 | S8P_C13R_D416N_R333A_G337A |
| DSGT-0715 | S8P_C13R_D416N_R333A_I338A |
| DSGT-0716 | S8P_C13R_D416N_N335A_R336A |
| DSGT-0717 | S8P_C13R_D416N_N335A_G337A |
| DSGT-0718 | S8P_C13R_D416N_N335A_I338A |
| DSGT-0719 | S8P_C13R_D416N_R336A_G337A |
| DSGT-0720 | S8P_C13R_D416N_R336A_I338A |
| DSGT-0721 | S8P_C13R_D416N_G337A_I338A |
| DSGT-0722 | S8P_C13R_D416N_R333K |
| DSGT-0723 | S8P_C13R_D416N_R333S |
| DSGT-0724 | S8P_C13R_D416N_R333K_R336K |
| DSGT-0725 | S8P_C13R_D416N_R333S_R336K |
| DSGT-0726 | S8P_C13R_D416N_R333K_R336S |
| DSGT-0727 | S8P_C13R_D416N_R333S_R336S |
| DSGT-0728 | S8P_C13R_D416N_R336K |
| DSGT-0729 | S8P_C13R_D416N_R336S |
| DSGT-0730 | S8P_C13R_D416N_R333K_K334A |
| DSGT-0731 | S8P_C13R_D416N_R333S_K334A |
| DSGT-0732 | S8P_C13R_D416N_R333K_K334A_R336K |
| DSGT-0733 | S8P_C13R_D416N_R333S_K334A_R336K |
| DSGT-0734 | S8P_C13R_D416N_R333K_K334A_R336S |
| DSGT-0735 | S8P_C13R_D416N_R333S_K334A_R336S |
| DSGT-0736 | S8P_C13R_D416N_K334A_R336K |
| DSGT-0737 | S8P_C13R_D416N_K334A_R336S |
| DSGT-0738 | S8P_C13R_D416N_R333K_N335A |
| DSGT-0739 | S8P_C13R_D416N_R333S_N335A |
| DSGT-0740 | S8P_C13R_D416N_R333K_N335A_R336K |
| DSGT-0741 | S8P_C13R_D416N_R333S_N335A_R336K |
| DSGT-0742 | S8P_C13R_D416N_R333K_N335A_R336S |
| DSGT-0743 | S8P_C13R_D416N_R333S_N335A_R336S |
| DSGT-0744 | S8P_C13R_D416N_N335A_R336K |
| DSGT-0745 | S8P_C13R_D416N_N335A_R336S |
| DSGT-0746 | S8P_C13R_D416N_R333K_K334A_N335A |
| DSGT-0747 | S8P_C13R_D416N_R333S_K334A_N335A |
| DSGT-0748 | S8P_C13R_D416N_R333K_K334A_N335A_R336K |
| DSGT-0749 | S8P_C13R_D416N_R333S_K334A_N335A_R336K |
| DSGT-0750 | S8P_C13R_D416N_R333K_K334A_N335A_R336S |
| DSGT-0751 | S8P_C13R_D416N_R333S_K334A_N335A_R336S |
| DSGT-0752 | S8P_C13R_D416N_K334A_N335A_R336K |
| DSGT-0753 | S8P_C13R_D416N_K334A_N335A_R336S |
| DSGT-0754 | S8P_C13R_D416N_K286A_R287A_R333A |
| DSGT-0755 | S8P_C13R_D416N_K286A_R287A_K334A |
| DSGT-0756 | S8P_C13R_D416N_K286A_R287A_N335A |
| DSGT-0757 | S8P_C13R_D416N_K286A_R287A_R336A |
| DSGT-0758 | S8P_C13R_D416N_K286A_R287A_N335A |
| DSGT-0759 | S8P_C13R_D416N_K286A_R287A_K334A_N335A |
| DSGT-0760 | S8P_C13R_D416N_K286A_R287A_K334A_R336A |
| DSGT-0761 | S8P_C13R_D416N_K286A_R287A_R333A_N335A |
| DSGT-0762 | S8P_C13R_D416N_K286A_R287A_R333A_R336A |
| DSGT-0763 | S8P_C13R_D416N_K286A_R287A_N335A_R336A |
| DSGT-0764 | D416N_Y281A |
| DSGT-0765 | D416N_C282A |
| DSGT-0766 | D416N_G283A |
| DSGT-0767 | D416N_E284A |
| DSGT-0768 | D416N_G285A |
| DSGT-0769 | D416N_K286A |
| DSGT-0770 | D416N_R287A |
| DSGT-0771 | D416N_L288A |
| DSGT-0772 | D416N_L289A |
| DSGT-0773 | D416N_E290A |
| DSGT-0774 | D416N_T291A |
| DSGT-0775 | D416N_I332A |
| DSGT-0776 | D416N_R333A |
| DSGT-0777 | D416N_K334A |
| DSGT-0778 | D416N_N335A |
| DSGT-0779 | D416N_R336A |
| DSGT-0780 | D416N_G337A |
| DSGT-0781 | D416N_I338A |
| DSGT-0782 | D416N_P339A |
| DSGT-0783 | D416N_G285A_K286A |
| DSGT-0784 | D416N_G285A_R287A |
| DSGT-0785 | D416N_G285A_I332A |
| DSGT-0786 | D416N_G285A_R333A |
| DSGT-0787 | D416N_G285A_N335A |
| DSGT-0788 | D416N_G285A_R336A |
| DSGT-0789 | D416N_G285A_G337A |
| DSGT-0790 | D416N_G285A_I338A |
| DSGT-0791 | D416N_K286A_R287A |
| DSGT-0792 | D416N_K286A_I332A |
| DSGT-0793 | D416N_K286A_R333A |
| DSGT-0794 | D416N_K286A_N335A |
| DSGT-0795 | D416N_K286A_R336A |
| DSGT-0796 | D416N_K286A_G337A |
| DSGT-0797 | D416N_K286A_I338A |
| DSGT-0798 | D416N_R287A_1332A |
| DSGT-0799 | D416N_R287A_R333A |
| DSGT-0800 | D416N_R287A_N335A |
| DSGT-0801 | D416N_R287A_R336A |
| DSGT-0802 | D416N_R287A_G337A |
| DSGT-0803 | D416N_R287A_I338A |
| DSGT-0804 | D416N_I332A_R333A |
| DSGT-0805 | D416N_I332A_N335A |
| DSGT-0806 | D416N_I332A_R336A |
| DSGT-0807 | D416N_1332A_G337A |
| DSGT-0808 | D416N_I332A_I338A |
| DSGT-0809 | D416N_R333A_N335A |
| DSGT-0810 | D416N_R333A_R336A |
| DSGT-0811 | D416N_R333A_G337A |
| DSGT-0812 | D416N_R333A_I338A |
| DSGT-0813 | D416N_N335A_R336A |
| DSGT-0814 | D416N_N335A_G337A |
| DSGT-0815 | D416N_N335A_I338A |
| DSGT-0816 | D416N_R336A_G337A |
| DSGT-0817 | D416N_R336A_I338A |
| DSGT-0818 | D416N_G337A_I338A |
| DSGT-0819 | D416N_R333K |
| DSGT-0820 | D416N_R333S |
| DSGT-0821 | D416N_R333K_R336K |
| DSGT-0822 | D416N_R333S_R336K |
| DSGT-0823 | D416N_R333K_R336S |
| DSGT-0824 | D416N_R333S_R336S |
| DSGT-0825 | D416N_R336K |
| DSGT-0826 | D416N_R336S |
| DSGT-0827 | D416N_R333K_K334A |
| DSGT-0828 | D416N_R333S_K334A |
| DSGT-0829 | D416N_R333K_K334A_R336K |
| DSGT-0830 | D416N_R333S_K334A_R336K |
| DSGT-0831 | D416N_R333K_K334A_R336S |
| DSGT-0832 | D416N_R333S_K334A_R336S |
| DSGT-0833 | D416N_K334A_R336K |
| DSGT-0834 | D416N_K334A_R336S |
| DSGT-0835 | D416N_R333K_N335A |
| DSGT-0836 | D416N_R333S_N335A |
| DSGT-0837 | D416N_R333K_N335A_R336K |
| DSGT-0838 | D416N_R333S_N335A_R336K |
| DSGT-0839 | D416N_R333K_N335A_R336S |
| DSGT-0840 | D416N_R333S_N335A_R336S |
| DSGT-0841 | D416N_N335A_R336K |
| DSGT-0842 | D416N_N335A_R336S |
| DSGT-0843 | D416N_R333K_K334A_N335A |
| DSGT-0844 | D416N_R333S_K334A_N335A |
| DSGT-0845 | D416N_R333K_K334A_N335A_R336K |
| DSGT-0846 | D416N_R333S_K334A_N335A_R336K |
| DSGT-0847 | D416N_R333K_K334A_N335A_R336S |
| DSGT-0848 | D416N_R333S_K334A_N335A_R336S |
| DSGT-0849 | D416N_K334A_N335A_R336K |
| DSGT-0850 | D416N_K334A_N335A_R336S |
| DSGT-0851 | S8P_C13R_D416N_Y281A |
| DSGT-0852 | S8P_C13R_D416N_Y281A_K286A_R287A |
| DSGT-0853 | S8P_C13R_D416N_Y281A_R333A |
| DSGT-0854 | S8P_C13R_D416N_Y281A_K334A |
| DSGT-0855 | S8P_C13R_D416N_Y281A_N335A |
| DSGT-0856 | S8P_C13R_D416N_Y281A_R336A |
| DSGT-0857 | S8P_C13R_D416N_Y281A_K286A_R287A_R333A |
| DSGT-0858 | S8P_C13R_D416N_Y281A_K286A_R287A_K334A |
| DSGT-0859 | S8P_C13R_D416N_Y281A_K286A_R287A_N335A |
| DSGT-0860 | S8P_C13R_D416N_Y281A_K286A_R287A_R336A |
| DSGT-0861 | S8P_C13R_D416N_Y281A_K286A_R287A_R333A_ |
| | K334A |
| DSGT-0862 | S8P_C13R_D416N_Y281A_K286A_R287A_K334A_ |
| | N335A |
| DSGT-0863 | S8P_C13R_D416N_Y281A_K286A_R287A_K334A_ |
| | R336A |
| DSGT-0864 | S8P_C13R_D416N_Y281A_K286A_R287A_R333A_ |
| | N335A |
| DSGT-0865 | S8P_C13R_D416N_Y281A_K286A_R287A_R333A_ |
| | R336A |
| DSGT-0870 | D416N_K334A |
| DSGT-0871 | D416N_K334A_N335A |
| DSGT-0872 | D416N_K334A_R336A |
| DSGT-0873 | D416N_N335A |
| DSGT-0874 | D416N_N335A_R336A |
| DSGT-0875 | D416N_R333A |
| DSGT-0876 | R333A_K334A |
| DSGT-0877 | D416N_R333A_N335A |
| DSGT-0878 | D416N_R333A_R336A |
| DSGT-0879 | D416N_R336A |
| DSGT-0880 | D416N_Y281A |
| DSGT-0881 | D416N_Y281A_K334A |
| DSGT-0882 | D416N_Y281A_K334A_N335A |
| DSGT-0883 | D416N_Y281A_K334A_R336A |
| DSGT-0884 | D416N_Y281A_N335A |
| DSGT-0885 | D416N_Y281A_N335A_R336A |
| DSGT-0886 | D416N_Y281A_R333A |
| DSGT-0887 | D416N_Y281A_R333A_K334A |
| DSGT-0888 | D416N_Y281A_R333A_K335A |
| DSGT-0889 | D416N_Y281A_R333A_R336A |
| DSGT-0890 | D416N_Y281A_R336A |
| DSGT-0954 | del2-35_D416N |
| DSGT-0955 | del2-35_D416N_K334A |
| DSGT-0956 | del2-35_D416N_K334A_N335A |
| DSGT-0957 | del2-35_D416N_K334A_R336A |
| DSGT-0958 | del2-35_D416N_N335A |
| DSGT-0959 | de12-35_D416N_N335A_R336A |
| DSGT-0960 | del2-35_D416N_R333A |
| DSGT-0961 | del2-35_D416N_R333A_K334A |
| DSGT-0962 | del2-35_D416N_R333A_N335A |
| DSGT-0963 | del2-35_D416N_R333A_R336A |
| DSGT-0964 | del2-35_D416N_R336A |
| DSGT-0965 | del2-35_D416N_Y281A |
| DSGT-0966 | del2-35_D416N_Y281A_K334A |
| DSGT-0967 | del2-35_D416N_Y281A_K334A_N335A |
| DSGT-0968 | del2-35_D416N_Y281A_K334A_R336A |
| DSGT-0969 | del2-35_D416N_Y281A_N335A |
| DSGT-0970 | del2-35_D416N_Y281A_N335A_R336A |
| DSGT-0971 | del2-35_D416N_Y281A_R333A |
| DSGT-0972 | del2-35_D416N_Y281A_R333A_N335A |
| DSGT-0973 | del2-35_D416N_Y281A_R333A_K334A |
| DSGT-0974 | del2-35_D416N_Y281A_R333A_R336A |
| DSGT-0975 | de12-35_D416N_Y281A_R336A |
| DSGT-0976 | del2-45_D416N |
| DSGT-0977 | del2-45_D416N_K334A |
| DSGT-0978 | del2-45_D416N_K334A_N335A |
| DSGT-0979 | del2-45_D416N_K334A_R336A |
| DSGT-0980 | del2-45_D416N_N335A |
| DSGT-0981 | del2-45_D416N_N335A_R336A |
| DSGT-0982 | del2-45_D416N_R333A |
| DSGT-0983 | del2-45_D416N_R333A_K334A |
| DSGT-0984 | de12-45_D416N_R333A_N335A |
| DSGT-0985 | del2-45_D416N_R333A_R336A |
| DSGT-0986 | del2-45_D416N_R336A |
| DSGT-0987 | del2-45_D416N_Y281A |
| DSGT-0988 | del2-45_D416N_Y281A_K334A |
| DSGT-0989 | del2-45_D416N_Y281A_K334A_N335A |
| DSGT-0990 | del2-45_D416N_Y281A_K334A_R336A |
| DSGT-0991 | del2-45_D416N_Y281A_N335A |
| DSGT-0992 | del2-45_D416N_Y281A_N335A_R336A |
| DSGT-0993 | del2-45_D416N_Y281A_R333A |
| DSGT-0994 | del2-45_D416N_Y281A_R333A_N335A |
| DSGT-0995 | del2-45_D416N_Y281A_R333A_K334A |
| DSGT-0996 | del2-45_D416N_Y281A_R333A_R336A |
| DSGT-0997 | del2-45_D416N_Y281A_R336A |
| DSGT-0998 | del2-68_D416N |
| DSGT-0999 | de12-68_D416N_K334A |
| DSGT-1000 | del2-68_D416N_K334A_N335A |
| DSGT-1001 | del2-68_D416N_K334A_R336A |
| DSGT-1002 | del2-68_D416N_N335A |
| DSGT-1003 | del2-68_D416N_N335A_R336A |
| DSGT-1004 | del2-68_D416N_R333A |
| DSGT-1005 | del2-68_D416N_R333A_K334A |
| DSGT-1006 | del2-68_D416N_R333A_N335A |
| DSGT-1007 | de12-68_D416N_R333A_R336A |
| DSGT-1008 | del2-68_D416N_R336A |
| DSGT-1009 | del2-68_D416N_Y281A |
| DSGT-1010 | del2-68_D416N_Y281A_K334A |
| DSGT-1011 | del2-68_D416N_Y281A_K334A_N335A |
| DSGT-1012 | del2-68_D416N_Y281A_K334A_R336A |
| DSGT-1013 | del2-68_D416N_Y281A_N335A |
| DSGT-1014 | del2-68_D416N_Y281A_N335A_R336A |
| DSGT-1015 | del2-68_D416N_Y281A_R333A |
| DSGT-1016 | del2-68_D416N_Y281A_R333A_N335A |
| DSGT-1017 | del2-68_D416N_Y281A_R333A_K334A |
| DSGT-1018 | del2-68_D416N_Y281A_R333A_R336A |
| DSGT-1019 | del2-68_D416N_Y281A_R336A |
| DSGT-1042 | S8P_C13R_D416N_K334A |
| DSGT-1043 | S8P_C13R_D416N_K334A_N335A |
| DSGT-1044 | S8P_C13R_D416N_K334A_R336A |
| DSGT-1045 | S8P_C13R_D416N_N335A |
| DSGT-1046 | S8P_C13R_D416N_N335A_R336A |
| DSGT-1047 | S8P_C13R_D416N_R333A |
| DSGT-1048 | S8P_C13R_D416N_R333A_K334A |
| DSGT-1049 | S8P_C13R_D416N_R333A_N335A |
| DSGT-1050 | S8P_C13R_D416N_R333A_R336A |
| DSGT-1051 | S8P_C13R_D416N_R336A |
| DSGT-1052 | S8P_C13R_D416N_Y281A |
| DSGT-1053 | S8P_C13R_D416N_Y281A_K334A |
| DSGT-1054 | S8P_C13R_D416N_Y281A_K334A_N335A |
| DSGT-1055 | S8P_C13R_D416N_Y281A_K334A_R336A |
| DSGT-1056 | S8P_C13R_D416N_Y281A_N335A |
| DSGT-1057 | S8P_C13R_D416N_Y281A_N335A_R336A |
| DSGT-1058 | S8P_C13R_D416N_Y281A_R333A |
| DSGT-1059 | S8P_C13R_D416N_Y281A_R333A_N335A |
| DSGT-1060 | S8P_C13R_D416N_Y281A_R333A_K334A |
| DSGT-1061 | S8P_C13R_D416N_Y281A_R333A_R336A |
| DSGT-1062 | S8P_C13R_D416N_Y281A_R336A |

TABLE 1A
Transposase helper mutations screened for Exc + Int-activity,
mutations relative to SEQ ID NO: 1

| ID | Mutations |
|---|---|
| 1001 | S8P/C13R/D416N/K334A/N335A/R336K |
| 1002 | S8P/C13R/D416N/K334A/N335A/R336S |
| 1003 | S8P/C13R/D416N/K334A/R336K |
| 1004 | S8P/C13R/D416N/K334A/R336S |
| 1005 | S8P/C13R/D416N/N335A/R336K |
| 1006 | S8P/C13R/D416N/N335A/R336S |
| 1007 | S8P/C13R/D416N/R333K |
| 1008 | S8P/C13R/D416N/R333K/K334A |
| 1009 | S8P/C13R/D416N/R333K/K334A/N335A |
| 1010 | S8P/C13R/D416N/R333K/K334A/N335A/R336K |
| 1011 | S8P/C13R/D416N/R333K/K334A/N335A/R336S |
| 1012 | S8P/C13R/D416N/R333K/K334A/R336A |
| 1013 | S8P/C13R/D416N/R333K/K334A/R336K |
| 1014 | S8P/C13R/D416N/R333K/K334A/R336S |
| 1015 | S8P/C13R/D416N/R333K/N335A |
| 1016 | S8P/C13R/D416N/R333K/N335A/R336K |
| 1017 | S8P/C13R/D416N/R333K/N335A/R336S |
| 1018 | S8P/C13R/D416N/R333K/R336K |
| 1019 | S8P/C13R/D416N/R333K/R336S |
| 1020 | S8P/C13R/D416N/R333S |
| 1021 | S8P/C13R/D416N/R333S/K334A |
| 1022 | S8P/C13R/D416N/R333S/K334A/N335A |
| 1023 | S8P/C13R/D416N/R333S/K334A/N335A/R336K |
| 1024 | S8P/C13R/D416N/R333S/K334A/N335A/R336S |
| 1025 | S8P/C13R/D416N/R333S/K334A/R336K |
| 1026 | S8P/C13R/D416N/R333S/K334A/R336S |
| 1027 | S8P/C13R/D416N/R333S/N335A |
| 1028 | S8P/C13R/D416N/R333S/N335A/R336K |
| 1029 | S8P/C13R/D416N/R333S/N335A/R336S |
| 1030 | S8P/C13R/D416N/R333S/R336K |
| 1031 | S8P/C13R/D416N/R333S/R336S |
| 1032 | S8P/C13R/D416N/R336K |
| 1033 | S8P/C13R/D416N/R336S |

TABLE 2
Exc + Int-mutant transposases with N-terminal zinc
finger (E2C) fusion, mutations relative to SEQ ID NO: 1
(with linker comprising optionally amino acid
sequence of SEQ ID NO: 8 or a variant thereof)

| ID | Mutations |
|---|---|
| DSGT-0891 | E2C_del2-35_D416N_K334A |
| DSGT-0892 | E2C_del2-35_D416N_K334A_N335A |
| DSGT-0893 | E2C_del2-35_D416N_K334A_R336A |
| DSGT-0894 | E2C_del2-35_D416N_N335A |
| DSGT-0895 | E2C_del2-35_D416N_N335A_R336A |
| DSGT-0896 | E2C_del2-35_D416N_R333A |
| DSGT-0897 | E2C_del2-35_D416N_R333A_K334A |
| DSGT-0898 | E2C_del2-35_D416N_R333A_N335A |
| DSGT-0899 | E2C_del2-35_D416N_R333A_R336A |
| DSGT-0900 | E2C_del2-35_D416N_R336A |
| DSGT-0901 | E2C_del2-35_D416N_Y281A |
| DSGT-0902 | E2C_del2-35_D416N_Y281A_K334A |
| DSGT-0903 | E2C_del2-35_D416N_Y281A_K334A_N335A |
| DSGT-0904 | E2C_del2-35_D416N_Y281A_K334A_R336A |
| DSGT-0905 | E2C_del2-35_D416N_Y281A_N335A |
| DSGT-0906 | E2C_del2-35_D416N_Y281A_N335A_R336A |
| DSGT-0907 | E2C_del2-35_D416N_Y281A_R333A |
| DSGT-0908 | E2C_del2-35_D416N_Y281A_R333A_N335A |
| DSGT-0909 | E2C_del2-35_D416N_Y281A_R333A_K334A |
| DSGT-0910 | E2C_del2-35_D416N_Y281A_R333A_R336A |
| DSGT-0911 | E2C_del2-35_D416N_Y281A_R336A |
| DSGT-0912 | E2C_del2-35_D416N_K334A |
| DSGT-0913 | E2C_del2-35_D416N_K334A_N335A |
| DSGT-0914 | E2C_del2-35_D416N_K334A_R336A |
| DSGT-0915 | E2C_del2-35_D416N_N335A |
| DSGT-0916 | E2C_del2-35_D416N_N335A_R336A |
| DSGT-0917 | E2C_del2-35_D416N_R333A |
| DSGT-0918 | E2C_del2-35_D416N_R333A_K334A |
| DSGT-0919 | E2C_del2-35_D416N_R333A_N335A |
| DSGT-0920 | E2C_del2-35_D416N_R333A_R336A |
| DSGT-0921 | E2C_del2-35_D416N_R336A |
| DSGT-0922 | E2C_del2-35_D416N_Y281A |
| DSGT-0923 | E2C_del2-35_D416N_Y281A_K334A |
| DSGT-0924 | E2C_del2-35_D416N_Y281A_K334A_N335A |
| DSGT-0925 | E2C_del2-35_D416N_Y281A_K334A_R336A |
| DSGT-0926 | E2C_del2-35_D416N_Y281A_N335A |
| DSGT-0927 | E2C_del2-35_D416N_Y281A_N335A_R336A |
| DSGT-0928 | E2C_del2-35_D416N_Y281A_R333A |
| DSGT-0929 | E2C_del2-35_D416N_Y281A_R333A_N335A |
| DSGT-0930 | E2C_del2-35_D416N_Y281A_R333A_K334A |
| DSGT-0931 | E2C_del2-35_D416N_Y281A_R333A_R336A |
| DSGT-0932 | E2C_del2-35_D416N_Y281A_R336A |
| DSGT-0933 | E2C_del2-35_D416N_K334A |
| DSGT-0934 | E2C_del2-35_D416N_K334A_N335A |
| DSGT-0935 | E2C_del2-35_D416N_K334A_R336A |
| DSGT-0936 | E2C_del2-35_D416N_N335A |
| DSGT-0937 | E2C_del2-35_D416N_N335A_R336A |
| DSGT-0938 | E2C_del2-35_D416N_R333A |
| DSGT-0939 | E2C_del2-35_D416N_R333A_K334A |
| DSGT-0940 | E2C_del2-35_D416N_R333A_N335A |
| DSGT-0941 | E2C_del2-35_D416N_R333A_R336A |
| DSGT-0942 | E2C_del2-35_D416N_R336A |
| DSGT-0943 | E2C_del2-35_D416N_Y281A |
| DSGT-0944 | E2C_del2-35_D416N_Y281A_K334A |
| DSGT-0945 | E2C_del2-35_D416N_Y281A_K334A_N335A |
| DSGT-0946 | E2C_del2-35_D416N_Y281A_K334A_R336A |
| DSGT-0947 | E2C_del2-35_D416N_Y281A_N335A |
| DSGT-0948 | E2C_del2-35_D416N_Y281A_N335A_R336A |
| DSGT-0949 | E2C_del2-35_D416N_Y281A_R333A |
| DSGT-0950 | E2C_del2-35_D416N_Y281A_R333A_N335A |
| DSGT-0951 | E2C_del2-35_D416N_Y281A_R333A_K334A |
| DSGT-0952 | E2C_del2-35_D416N_Y281A_R333A_R336A |
| DSGT-0953 | E2C_del2-35_D416N_Y281A_R336A |
| DSGT-1020 | E2C_S8P_C13R_D416N |
| DSGT-1021 | E2C_S8P_C13R_D416N_K334A |
| DSGT-1022 | E2C_S8P_C13R_D416N_K334A_N335A |
| DSGT-1023 | E2C_S8P_C13R_D416N_K334A_R336A |
| DSGT-1024 | E2C_S8P_C13R_D416N_N335A |
| DSGT-1025 | E2C_S8P_C13R_D416N_N335A_R336A |
| DSGT-1026 | E2C_S8P_C13R_D416N_R333A |
| DSGT-1027 | E2C_S8P_C13R_D416N_R333A_K334A |
| DSGT-1028 | E2C_S8P_C13R_D416N_R333A_N335A |
| DSGT-1029 | E2C_S8P_C13R_D416N_R333A_R336A |
| DSGT-1030 | E2C_S8P_C13R_D416N_R336A |
| DSGT-1031 | E2C_S8P_C13R_D416N_Y281A |
| DSGT-1032 | E2C_S8P_C13R_D416N_Y281A_K334A |
| DSGT-1033 | E2C_S8P_C13R_D416N_Y281A_K334A_N335A |
| DSGT-1034 | E2C_S8P_C13R_D416N_Y281A_K334A_R336A |
| DSGT-1035 | E2C_S8P_C13R_D416N_Y281A_N335A |
| DSGT-1036 | E2C_S8P_C13R_D416N_Y281A_N335A_R336A |
| DSGT-1037 | E2C_S8P_C13R_D416N_Y281A_R333A |
| DSGT-1038 | E2C_S8P_C13R_D416N_Y281A_R333A_N335A |
| DSGT-1039 | E2C_S8P_C13R_D416N_Y281A_R333A_K334A |
| DSGT-1040 | E2C_S8P_C13R_D416N_Y281A_R333A_R336A |
| DSGT-1041 | E2C_S8P_C13R_D416N_Y281A_R336A |

TABLE 2A
Exc + Int-mutant transposases with N-terminal zinc finger
(E2C) fusion, mutations relative to SEQ ID NO: 1 (with
linker comprising amino acid sequence of SEQ ID NO: 8)

| ID | Mutations |
|---|---|
| 2001 | E2C/S8P/C13R/D416N/K334A/N335A/R336K |
| 2002 | E2C/S8P/C13R/D416N/K334A/N335A/R336S |
| 2003 | E2C/S8P/C13R/D416N/K334A/R336K |
| 2004 | E2C/S8P/C13R/D416N/K334A/R336S |
| 2005 | E2C/S8P/C13R/D416N/N335A/R336K |
| 2006 | E2C/S8P/C13R/D416N/N335A/R336S |
| 2007 | E2C/S8P/C13R/D416N/R333K |
| 2008 | E2C/S8P/C13R/D416N/R333K/K334A |
| 2009 | E2C/S8P/C13R/D416N/R333K/K334A/N335A |
| 2020 | E2C/S8P/C13R/D416N/R333K/K334A/N335A/R336K |
| 2011 | E2C/S8P/C13R/D416N/R333K/K334A/N335A/R336S |
| 2012 | E2C/S8P/C13R/D416N/R333K/K334A/R336A |
| 2013 | E2C/S8P/C13R/D416N/R333K/K334A/R336K |
| 2014 | E2C/S8P/C13R/D416N/R333K/K334A/R336S |
| 2015 | E2C/S8P/C13R/D416N/R333K/N335A |
| 2016 | E2C/S8P/C13R/D416N/R333K/N335A/R336K |
| 2017 | E2C/S8P/C13R/D416N/R333K/N335A/R336S |
| 2018 | E2C/S8P/C13R/D416N/R333K/R336K |
| 2019 | E2C/S8P/C13R/D416N/R333K/R336S |
| 2020 | E2C/S8P/C13R/D416N/R333S |
| 2021 | E2C/S8P/C13R/D416N/R333S/K334A |
| 2022 | E2C/S8P/C13R/D416N/R333S/K334A/N335A |
| 2023 | E2C/S8P/C13R/D416N/R333S/K334A/N335A/R336K |
| 2024 | E2C/S8P/C13R/D416N/R333S/K334A/N335A/R336S |
| 2025 | E2C/S8P/C13R/D416N/R333S/K334A/R336K |
| 2026 | E2C/S8P/C13R/D416N/R333S/K334A/R336S |
| 2027 | E2C/S8P/C13R/D416N/R333S/N335A |
| 2028 | E2C/S8P/C13R/D416N/R333S/N335A/R336K |
| 2029 | E2C/S8P/C13R/D416N/R333S/N335A/R336S |
| 2030 | E2C/S8P/C13R/D416N/R333S/R336K |
| 2031 | E2C/S8P/C13R/D416N/R333S/R336S |
| 2032 | E2C/S8P/C13R/D416N/R336K |
| 2033 | E2C/S8P/C13R/D416N/R336S |

### Engineered Helper Enzyme

In embodiments, the helper enzyme further comprises a zinc finger (E2C) fused to the N-terminus and/or C-terminus. In embodiments, the helper enzyme further comprises a zinc finger (E2C) fused to the N-terminus. In embodiments, the helper enzyme further comprises a zinc finger (E2C) inserted after the glycine at amino acid position 66 relative to SEQ ID NO: 1 within the N-terminus. In embodiments, the helper enzyme is linked to a zinc finger (E2C) using recombinant variable domains of heavy-chain-only antibodies (VHH) (NANOBODY) bound to an ALFA-tag peptide.

In embodiments, the helper enzyme further comprises a zinc finger inserted after a residue selected from S60-E70 relative to SEQ ID NO: 1. In embodiments, the helper enzyme further comprises a zinc finger inserted after the G66 residue relative to SEQ ID NO: 1. In embodiments, the helper enzyme further comprises a zinc finger inserted after the S60 residue relative to SEQ ID NO: 1. In embodiments, the helper enzyme further comprises a zinc finger inserted after the E70 residue relative to SEQ ID NO: 1. In embodiments, the helper enzyme further comprises a zinc finger inserted after the L80 residue relative to SEQ ID NO: 1. In embodiments, the helper enzyme further comprises a zinc finger inserted after the N90 residue relative to SEQ ID NO: 1. In embodiments, the helper enzyme further comprises a zinc finger inserted after the D100 residue relative to SEQ ID NO: 1.

In embodiments, the helper enzyme further comprises a zinc finger inserted after the G66 residue relative to SEQ ID NO: 1 with mutations selected from one or more mutations, S8P, C13R, R333K, K334A, N335A, R336K, and/or D416N relative to SEQ ID NO: 1. In embodiments, the helper enzyme further comprises a zinc finger inserted immediately after the G66 residue relative to SEQ ID NO: 1 with mutations selected from one or more mutations, S8P, C13R, R333K, K334A, N335A, R336K, and/or D416N relative to SEQ ID NO: 1.

In embodiments, the helper enzyme further comprises a zinc finger inserted after the G66 residue relative to SEQ ID NO: 1 with mutations S8P, C13R, R333K, K334A, N335A, R336K, and D416N relative to SEQ ID NO: 1. In embodiments, the helper enzyme further comprises a zinc finger inserted immediately after the G66 residue relative to SEQ ID NO: 1 with mutations S8P, C13R, R333K, K334A, N335A, R336K, and D416N relative to SEQ ID NO: 1.

In embodiments, the helper enzyme further comprises a zinc finger inserted after the G66 residue relative to SEQ ID NO: 1 with mutations S8P, C13R, R333K, N335A, R336K, and D416N relative to SEQ ID NO: 1. In embodiments, the helper enzyme further comprises a zinc finger inserted immediately after the G66 residue relative to SEQ ID NO: 1 with mutations S8P, C13R, R333K, N335A, R336K, and D416N relative to SEQ ID NO: 1.

In embodiments, the helper enzyme and DNA binder comprises an amino acid sequence of at least about 90% identity to SEQ ID NO: 882. In embodiments, the helper enzyme and DNA binder comprises an amino acid sequence of at least about 93% identity to SEQ ID NO: 882. In embodiments, the helper enzyme and DNA binder comprises an amino acid sequence of at least about 95% identity to SEQ ID NO: 882. In embodiments, the helper enzyme and DNA binder comprises an amino acid sequence of at least about 98% identity to SEQ ID NO: 882. In embodiments, the helper enzyme and DNA binder comprises an amino acid sequence of at least about 99% identity to SEQ ID NO: 882.

SEQ ID NO: 882: E2C ZnF inserted after the G66 residue relative
to SEQ ID NO: 1 of MLT with mutations,
S8P_C13R_R333K_K334A_N335A_R336K_D416N substitutions relative to SEQ ID NO: 1
ATGCCGAAAAAAAAACGAAAGGTGTACCCCTACGATGTACCGGACTATGCAGGAAGCGCCCAGCACAGCGACTACCC
CGACGACGAGTTCAGAGCCGATAAGCTGAGTAACTACAGCTGCGACAGCGACCTGGAAAACGCCAGCACATCCGACG
AGGACAGCTCTGACGACGAGGTGATGGTGCGGCCCAGAACCCTGAGACGGAGAAGAATCAGCAGCTCTAGCAGCGAC
TCTGAATCCGACATCGAGGGCGGAGGTCTCGAACCAGGCGAGAAGCCTTATGCCTGTCCTGAGTGTGGCAAATCCTT
CTCAAGAAAAGACTCTCTGGTTAGACACCAGAGAACACATACAGGGGAGAAACCCTATAAATGCCCCGAATGCGGAA
AGTCCTTTTCCCAGAGCGGCGATCTCCGGAGGCATCAGAGAACTCATACAGGCGAGAAACCATATAAGTGCCCCGAG
TGTGGGAAATCCTTTTCCGATTGTAGAGACCTGGCCAGACATCAAAGGACACATACAGGCAAGAAGACCGGAGGTGG
CCGGGAAGAGTGGAGCCACGTGGACAACCCTCCTGTTCTGGAAGATTTTCTGGGCCATCAGGGCCTGAACACCGACG
CCGTGATCAACAACATCGAGGATGCCGTGAAGCTGTTCATAGGAGATGATTTCTTTGAGTTCCTGGTCGAGGAATCC
AACCGCTATTACAACCAGAATAGAAACAACTTCAAGCTGAGCAAGAAAAGCCTGAAGTGGAAGGACATCACCCCTCA
GGAGATGAAAAAGTTCCTGGGACTGATCGTTCTGATGGGACAGGTGCGGAAGGACAGAAGGGATGATTACTGGACAA
CCGAACCTTGGACCGAGACCCCTTACTTTGGCAAGACCATGACCAGAGACAGATTCAGACAGATCTGGAAAGCCTGG
CACTTCAACAACAATGCTGATATCGTGAACGAGTCTGATAGACTGTGTAAAGTGCGGCCAGTGTTGGATTACTTCGT
GCCTAAGTTCATCAACATCTATAAGCCTCACCAGCAGCTGAGCCTGGATGAAGGCATCGTGCCCTGGCGGGGCAGAC
TGTTCTTCAGAGTGTACAATGCTGGCAAGATCGTCAAATACGGCATCCTGGTGCGCCTTCTGTGCGAGAGCGATACA
GGCTACATCTGTAATATGGAAATCTACTGCGGCGAGGGCAAAAGACTGCTGGAAACCATCCAGACCGTCGTTTCCCC
TTATACCGACAGCTGGTACCACATCTACATGGACAACTACTACAATTCTGTGGCCAACTGCGAGGCCCTGATGAAGA
ACAAGTTTAGAATCTGCGGCACAATCAAGGCCGCCAAGGGCATCCCTAAGGACTTCCAGACCATCTCTCTGAAGAAG
GGCGAAACCAAGTTCATCAGAAAGAACGACATCCTGCTCCAAGTGTGGCAGTCCAAGAAACCCGTGTACCTGATCAG
CAGCATCCATAGCGCCGAGATGGAAGAAAGCCAGAACATCGACAGAACAAGCAAGAAGAAGATCGTGAAGCCCAATG
CTCTGATCGACTACAACAAGCACATGAAAGGCGTGGACCGGGCCAACCAGTACCTGTCTTATTACTCTATCCTGAGA
AGAACAGTGAAATGGACCAAGAGACTGGCCATGTACATGATCAATTGCGCCCTGTTCAACAGCTACGCCGTGTACAA
GTCCGTGCGACAAAGAAAAATGGGATTCAAGATGTTCCTGAAGCAGACAGCCATCCACTGGCTGACAGACGACATTC
CTGAGGACATGGACATTGTGCCAGATCTGCAACCTGTGCCCAGCACCTCTGGTATGAGAGCTAAGCCTCCCACCAGC
GATCCTCCATGTAGACTGAGCATGGACATGCGGAAGCACACCCTGCAGGCCATCGTCGGCAGCGGCAAGAAGAAGAA
CATCCTTAGACGGTGCAGGGTGTGCAGCGTGCACAAGCTGCGGAGCGAGACTCGGTACATGTGCAAGTTTTGCAACA
TTCCCCTGCACAAGGGAGCCTGCTTCGAGAAGTACCACACCCTGAAGAATTAC

In embodiments, the helper enzyme and DNA binder comprises an amino acid sequence of at least about 90% identity to SEQ ID NO: 883. In embodiments, the helper enzyme and DNA binder comprises an amino acid sequence of at least about 93% identity to SEQ ID NO: 883. In embodiments, the helper enzyme and DNA binder comprises an amino acid sequence of at least about 95% identity to SEQ ID NO: 883. In embodiments, the helper enzyme and DNA binder comprises an amino acid sequence of at least about 98% identity to SEQ ID NO: 883. In embodiments, the helper enzyme and DNA binder comprises an amino acid sequence of at least about 99% identity to SEQ ID NO: 883.

SEQ ID NO: 883: E2C ZnF inserted after the G66 residue relative to
SEQ ID NO: 1 of MLT with mutations,
S8P_C13R_R333K_N335A_R336K_D416N substitutions relative to SEQ ID NO: 1
ATGCCGAAAAAAAAACGAAAGGTGTACCCCTACGATGTACCGGACTATGCAGGAAGCGCCCAGCACAGCGACTACCC
CGACGACGAGTTCAGAGCCGATAAGCTGAGTAACTACAGCTGCGACAGCGACCTGGAAAACGCCAGCACATCCGACG
AGGACAGCTCTGACGACGAGGTGATGGTGCGGCCCAGAACCCTGAGACGGAGAAGAATCAGCAGCTCTAGCAGCGAC
TCTGAATCCGACATCGAGGGCGGAGGTCTCGAACCAGGCGAGAAGCCTTATGCCTGTCCTGAGTGTGGCAAATCCTT
CTCAAGAAAAGACTCTCTGGTTAGACACCAGAGAACACATACAGGGGAGAAACCCTATAAATGCCCCGAATGCGGAA
AGTCCTTTTCCCAGAGCGGCGATCTCCGGAGGCATCAGAGAACTCATACAGGCGAGAAACCATATAAGTGCCCCGAG
TGTGGGAAATCCTTTTCCGATTGTAGAGACCTGGCCAGACATCAAAGGACACATACAGGCAAGAAGACCGGAGGTGG
CCGGGAAGAGTGGAGCCACGTGGACAACCCTCCTGTTCTGGAAGATTTTCTGGGCCATCAGGGCCTGAACACCGACG
CCGTGATCAACAACATCGAGGATGCCGTGAAGCTGTTCATAGGAGATGATTTCTTTGAGTTCCTGGTCGAGGAATCC
AACCGCTATTACAACCAGAATAGAAACAACTTCAAGCTGAGCAAGAAAAGCCTGAAGTGGAAGGACATCACCCCTCA
GGAGATGAAAAAGTTCCTGGGACTGATCGTTCTGATGGGACAGGTGCGGAAGGACAGAAGGGATGATTACTGGACAA
CCGAACCTTGGACCGAGACCCCTTACTTTGGCAAGACCATGACCAGAGACAGATTCAGACAGATCTGGAAAGCCTGG
CACTTCAACAACAATGCTGATATCGTGAACGAGTCTGATAGACTGTGTAAAGTGCGGCCAGTGTTGGATTACTTCGT
GCCTAAGTTCATCAACATCTATAAGCCTCACCAGCAGCTGAGCCTGGATGAAGGCATCGTGCCCTGGCGGGGCAGAC
TGTTCTTCAGAGTGTACAATGCTGGCAAGATCGTCAAATACGGCATCCTGGTGCGCCTTCTGTGCGAGAGCGATACA
GGCTACATCTGTAATATGGAAATCTACTGCGGCGAGGGCAAAAGACTGCTGGAAACCATCCAGACCGTCGTTTCCCC
TTATACCGACAGCTGGTACCACATCTACATGGACAACTACTACAATTCTGTGGCCAACTGCGAGGCCCTGATGAAGA
ACAAGTTTAGAATCTGCGGCACAATCAAGAAAGCCAAGGGCATCCCTAAGGACTTCCAGACCATCTCTCTGAAGAAG
GGCGAAACCAAGTTCATCAGAAAGAACGACATCCTGCTCCAAGTGTGGCAGTCCAAGAAACCCGTGTACCTGATCAG
CAGCATCCATAGCGCCGAGATGGAAGAAAGCCAGAACATCGACAGAACAAGCAAGAAGAAGATCGTGAAGCCCAATG
CTCTGATCGACTACAACAAGCACATGAAAGGCGTGGACCGGGCCAACCAGTACCTGTCTTATTACTCTATCCTGAGA
AGAACAGTGAAATGGACCAAGAGACTGGCCATGTACATGATCAATTGCGCCCTGTTCAACAGCTACGCCGTGTACAA
GTCCGTGCGACAAAGAAAAATGGGATTCAAGATGTTCCTGAAGCAGACAGCCATCCACTGGCTGACAGACGACATTC
CTGAGGACATGGACATTGTGCCAGATCTGCAACCTGTGCCCAGCACCTCTGGTATGAGAGCTAAGCCTCCCACCAGC
GATCCTCCATGTAGACTGAGCATGGACATGCGGAAGCACACCCTGCAGGCCATCGTCGGCAGCGGCAAGAAGAAGAA
CATCCTTAGACGGTGCAGGGTGTGCAGCGTGCACAAGCTGCGGAGCGAGACTCGGTACATGTGCAAGTTTTGCAACA
TTCCCCTGCACAAGGGAGCCTGCTTCGAGAAGTACCACACCCTGAAGAATTAC

In embodiments, the helper enzyme further comprises a TALE fused to the N-terminus and/or C-terminus. In embodiments, the helper enzyme further comprises a TALE fused to the N-terminus. In embodiments, the helper enzyme further comprises a TALE inserted after the glycine at amino acid position 66 relative to SEQ ID NO: 1 within the N-terminus. In embodiments, the helper enzyme is linked to a TALE using recombinant variable domains of heavy-chain-only antibodies (VHH) (NANOBODY) bound to an ALFA-tag peptide. In embodiments, the helper enzyme tethered to a TALE using recombinant variable domains of heavy-chain-only antibodies (VHH) (NANOBODY) bound to an ALFA-tag peptide can bind different recognition sequences spaced about 15, or about 16, or about 17, or about 18, or about 19, or about 20 base pairs on either side of the target.

In embodiments, the helper enzyme further comprises a TALE inserted after the G66 residue relative to SEQ ID NO: 1. In embodiments, the helper enzyme further comprises a TALE inserted after the G66 residue relative to SEQ ID NO: 1 with mutations selected from one or more mutations, S8P, C13R, R333K, K334A, N335A, R336K, and/or D416N relative to SEQ ID NO: 1.

In embodiments, the helper enzyme further comprises a TALE inserted after the G66 residue relative to SEQ ID NO: 1 with mutations S8P, C13R, R333K, N335A, R336K, and D416N relative to SEQ ID NO: 1.

In embodiments, the helper enzyme further comprises a TALE inserted immediately after the G66 residue relative to SEQ ID NO: 1. In embodiments, the helper enzyme further comprises a TALE inserted immediately after the G66 residue relative to SEQ ID NO: 1 with mutations selected from one or more mutations, S8P, C13R, R333K, K334A, N335A, R336K, and/or D416N relative to SEQ ID NO: 1.

In embodiments, the helper enzyme further comprises a TALE inserted immediately after the G66 residue relative to SEQ ID NO: 1 with mutations S8P, C13R, R333K, N335A, R336K, and D416N relative to SEQ ID NO: 1.

In embodiments, the helper enzyme and DNA binder comprises an amino acid sequence of at least about 90% identity to SEQ ID NO: 884. In embodiments, the helper enzyme and DNA binder comprises an amino acid sequence of at least about 93% identity to SEQ ID NO: 884. In embodiments, the helper enzyme and DNA binder comprises an amino acid sequence of at least about 95% identity to SEQ ID NO: 884. In embodiments, the helper enzyme and DNA binder comprises an amino acid sequence of at least about 98% identity to SEQ ID NO: 884. In embodiments, the helper enzyme and DNA binder comprises an amino acid sequence of at least about 99% identity to SEQ ID NO: 884.

SEQ ID NO: 884: HoD4 TALE inserted after the G66 residue relative to
SEQ ID NO: 1 of MLT with mutation:
R333K_N335A_R336K_D416N substitutions relative to SEQ ID NO: 1
ATGGCGCCTAAGAAGAAGCGCAAGGTGGGCGGAGCCCAGCACAGCGACTACCCCGACGACGAGTTCAGAGCCGATAA
GCTGAGTAACTACAGCTGCGACAGCGACCTGGAAAACGCCAGCACATCCGACGAGGACAGCTCTGACGACGAGGTGA
TGGTGCGGCCCAGAACCCTGAGACGGAGAAGAATCAGCAGCTCTAGCAGCGACTCTGAATCCGACATCGAGGGCGGT
GGTGGGTCTGTTGACCTCCGGACTTTGGGTTACTCCCAACAGCAACAAGAGAAGATTAAACCTAAGGTGCGCTCAAC
AGTGGCGCAGCATCATGAGGCACTGGTTGGTCACGGGTTCACACATGCCCATATAGTAGCACTTTCACAACATCCCG
CTGCACTCGGAACGGTTGCAGTAAAGTACCAAGACATGATTGCCGCATTGCCGGAAGCAACTCATGAGGCCATTGTC
GGAGTAGGCAAATACCACGGTGCTAGAGCTCTCGAAGCACTCCTCACTGTGGCTGGCGAATTGAGGGGGCCCCCCCT
TCAACTTGATACTGGACAGCTGTTGAAGATAGCGAAACGAGGGGGAGTAACCGCAGTCGAGGCCGTCCACGCGTGGC
GAAATGCATTGACGGGAGCGCCTCTCAACCTTACTCCAGATCAGGTTGTGGCCATAGCGAGCAACATCGGAGGTAAG
CAAGCACTGGAAACTGTTCAACGATTGCTCCCAGTACTCTGCCAAGACCACGGATTGACCCCAGAACAGGTTGTGGC
TATTGCCAGCAACGGCGGCGGTAAACAGGCTCTGGAAACTGTGCAGCGACTCTTGCCGGTGTTGTGCCAAGCTCATG
GCCTTACGCCAGACCAGGTTGTTGCTATCGCTAGCAACATCGGAGGCAAACAGGCACTTGAAACGGTCCAACGCCTC
TTGCCAGTATTGTGCCAGGCACATGGCCTCACCCCCGCCCAGGTTGTGGCCATCGCAAACAATAACGGGGGCAAGCA
GGCTTTGGAGACAGTCCAACGATTGTTGCCGGTACTTTGTCAGGACCACGGGCTGACCCCAGATCAAGTCGTAGCAA
TCGCTAGCAACATCGGCGGGAAGCAGGCCCTGGAAACGGTGCAGAGGCTCCTCCCAGTGCTCTGCCAAGATCATGGC
CTCACCCCAGAGCAAGTGGTCGCAATCGCAAACAATAACGGTGGCAAGCAAGCCCTCGAAACTGTGCAAAGGTTGCT
GCCAGTTCTCTGTCAAGCCCACGGTTTGACACCGGATCAGGTAGTGGCTATCGCTAGCAACGGCGGTGGCAAGCAAG
CTCTCGAGACAGTCCAGAGGCTTTTGCCAGTACTGTGCCAAGCGCACGGTCTCACTCCTGCGCAAGTTGTTGCTATA
GCAAACAATAACGGTGGAAAGCAAGCTCTTGAGACTGTGCAGCGCCTGTTGCCCGTCCTCTGTCAGGACCACGGTTT
GACTCCAGACCAAGTTGTCGCCATTGCAAGCAACGGCGGTGGACGCCCTGCACTGGAGTCCATCGTCGCACAACTGT
CTAGGCCGGACCCCGCCCTGGCTGCCCTTACTAATGATCACTTGGTTGCATTGGCGTGTCTGGGCGGTCGGCCAGCG
CTGGACGCAGTCAAGAAGGGACTCCCCCACGCCCCTGCACTTATAAAACGCACTAACAGGAGAATACCAGAACGGAC
GAGTCATCGAGTCGCTGGAGGGGGGGGTAGCGGCCGGGAAGAGTGGAGCCACGTGGACAACCCTCCTGTTCTGGAAG
ATTTTCTGGGCCATCAGGGCCTGAACACCGACGCCGTGATCAACAACATCGAGGATGCCGTGAAGCTGTTCATAGGA
GATGATTTCTTTGAGTTCCTGGTCGAGGAATCCAACCGCTATTACAACCAGAATAGAAACAACTTCAAGCTGAGCAA
GAAAAGCCTGAAGTGGAAGGACATCACCCCTCAGGAGATGAAAAAGTTCCTGGGACTGATCGTTCTGATGGGACAGG
TGCGGAAGGACAGAAGGGATGATTACTGGACAACCGAACCTTGGACCGAGACCCCTTACTTTGGCAAGACCATGACC
AGAGACAGATTCAGACAGATCTGGAAAGCCTGGCACTTCAACAACAATGCTGATATCGTGAACGAGTCTGATAGACT
GTGTAAAGTGCGGCCAGTGTTGGATTACTTCGTGCCTAAGTTCATCAACATCTATAAGCCTCACCAGCAGCTGAGCC
TGGATGAAGGCATCGTGCCCTGGCGGGGCAGACTGTTCTTCAGAGTGTACAATGCTGGCAAGATCGTCAAATACGGC
ATCCTGGTGCGCCTTCTGTGCGAGAGCGATACAGGCTACATCTGTAATATGGAAATCTACTGCGGCGAGGGCAAAAG
ACTGCTGGAAACCATCCAGACCGTCGTTTCCCCTTATACCGACAGCTGGTACCACATCTACATGGACAACTACTACA
ATTCTGTGGCCAACTGCGAGGCCCTGATGAAGAACAAGTTTAGAATCTGCGGCACAATCAGAAAAAACAGAGGCATC
CCTAAGGACTTCCAGACCATCTCTCTGAAGAAGGGCGAAACCAAGTTCATCAGAAAGAACGACATCCTGCTCCAAGT
GTGGCAGTCCAAGAAACCCGTGTACCTGATCAGCAGCATCCATAGCGCCGAGATGGAAGAAAGCCAGAACATCGACA
GAACAAGCAAGAAGAAGATCGTGAAGCCCAATGCTCTGATCGACTACAACAAGCACATGAAAGGCGTGGACCGGGCC
GACCAGTACCTGTCTTATTACTCTATCCTGAGAAGAACAGTGAAATGGACCAAGAGACTGGCCATGTACATGATCAA
TTGCGCCCTGTTCAACAGCTACGCCGTGTACAAGTCCGTGCGACAAAGAAAAATGGGATTCAAGATGTTCCTGAAGC
AGACAGCCATCCACTGGCTGACAGACGACATTCCTGAGGACATGGACATTGTGCCAGATCTGCAACCTGTGCCCAGC
ACCTCTGGTATGAGAGCTAAGCCTCCCACCAGCGATCCTCCATGTAGACTGAGCATGGACATGCGGAAGCACACCCT
GCAGGCCATCGTCGGCAGCGGCAAGAAGAAGAACATCCTTAGACGGTGCAGGGTGTGCAGCGTGCACAAGCTGCGGA
GCGAGACTCGGTACATGTGCAAGTTTTGCAACATTCCCCTGCACAAGGGAGCCTGCTTCGAGAAGTACCACACCCTG
AAGAATTAC

In embodiments, the helper enzyme further comprises an ALFA tag inserted after the G66 residue relative to SEQ ID NO: 1. In embodiments, the helper enzyme further comprises an ALFA tag inserted immediately after the G66 residue relative to SEQ ID NO: 1. In embodiments, the helper enzyme further comprises an ALFA tag. In embodiments, the ALFA tag comprises an amino acid sequence of SRLEEELRRRLTE (SEQ ID NO: 892), or a variant thereof having a substitution or deletion.

In embodiments, the ALFA tag inserted after the G66 residue of MLT comprises an amino acid sequence of at least about 90% identity, or at least about 93% identity, or at least about 95% identity, or at least about 98% identity, or at least about 99% identity to SEQ ID NO: 885. In embodiments, the ALFA tag inserted immediately after the G66 residue of MLT comprises an amino acid sequence of at least about 90% identity, or at least about 93% identity, or at least about 95% identity, or at least about 98% identity, or at least about 99% identity to SEQ ID NO: 885.

SEQ ID NO: 885: ALFA tag inserted after the G66 residue
relative to SEQ ID NO: 1 of MLT
ATGCCGAAAAAAAAACGAAAGGTGTACCCCTACGATGTACCGGACTATGCAGGAAGCGCCCAGCACAGCGACTACCC
CGACGACGAGTTCAGAGCCGATAAGCTGAGTAACTACAGCTGCGACAGCGACCTGGAAAACGCCAGCACATCCGACG
AGGACAGCTCTGACGACGAGGTGATGGTGCGGCCCAGAACCCTGAGACGGAGAAGAATCAGCAGCTCTAGCAGCGAC
TCTGAATCCGACATCGAGGGCTCCAGACTGGAAGAGGAACTGAGAAGAAGGCTCACAGAAGGCCGGGAAGAGTGGAG
CCACGTGGACAACCCTCCTGTTCTGGAAGATTTTCTGGGCCATCAGGGCCTGAACACCGACGCCGTGATCAACAACA
TCGAGGATGCCGTGAAGCTGTTCATAGGAGATGATTTCTTTGAGTTCCTGGTCGAGGAATCCAACCGCTATTACAAC
CAGAATAGAAACAACTTCAAGCTGAGCAAGAAAAGCCTGAAGTGGAAGGACATCACCCCTCAGGAGATGAAAAAGTT
CCTGGGACTGATCGTTCTGATGGGACAGGTGCGGAAGGACAGAAGGGATGATTACTGGACAACCGAACCTTGGACCG
AGACCCCTTACTTTGGCAAGACCATGACCAGAGACAGATTCAGACAGATCTGGAAAGCCTGGCACTTCAACAACAAT
GCTGATATCGTGAACGAGTCTGATAGACTGTGTAAAGTGCGGCCAGTGTTGGATTACTTCGTGCCTAAGTTCATCAA
CATCTATAAGCCTCACCAGCAGCTGAGCCTGGATGAAGGCATCGTGCCCTGGCGGGGCAGACTGTTCTTCAGAGTGT
ACAATGCTGGCAAGATCGTCAAATACGGCATCCTGGTGCGCCTTCTGTGCGAGAGCGATACAGGCTACATCTGTAAT
ATGGAAATCTACTGCGGCGAGGGCAAAAGACTGCTGGAAACCATCCAGACCGTCGTTTCCCCTTATACCGACAGCTG
GTACCACATCTACATGGACAACTACTACAATTCTGTGGCCAACTGCGAGGCCCTGATGAAGAACAAGTTTAGAATCT
GCGGCACAATCAGAAAAAACAGAGGCATCCCTAAGGACTTCCAGACCATCTCTCTGAAGAAGGGCGAAACCAAGTTC
ATCAGAAAGAACGACATCCTGCTCCAAGTGTGGCAGTCCAAGAAACCCGTGTACCTGATCAGCAGCATCCATAGCGC
CGAGATGGAAGAAAGCCAGAACATCGACAGAACAAGCAAGAAGAAGATCGTGAAGCCCAATGCTCTGATCGACTACA
ACAAGCACATGAAAGGCGTGGACCGGGCCGACCAGTACCTGTCTTATTACTCTATCCTGAGAAGAACAGTGAAATGG
ACCAAGAGACTGGCCATGTACATGATCAATTGCGCCCTGTTCAACAGCTACGCCGTGTACAAGTCCGTGCGACAAAG
AAAAATGGGATTCAAGATGTTCCTGAAGCAGACAGCCATCCACTGGCTGACAGACGACATTCCTGAGGACATGGACA
TTGTGCCAGATCTGCAACCTGTGCCCAGCACCTCTGGTATGAGAGCTAAGCCTCCCACCAGCGATCCTCCATGTAGA
CTGAGCATGGACATGCGGAAGCACACCCTGCAGGCCATCGTCGGCAGCGGCAAGAAGAAGAACATCCTTAGACGGTG
CAGGGTGTGCAGCGTGCACAAGCTGCGGAGCGAGACTCGGTACATGTGCAAGTTTTGCAACATTCCCCTGCACAAGG
GAGCCTGCTTCGAGAAGTACCACACCCTGAAGAATTAC

In embodiments, the DNA binder of the present disclosure further comprises recombinant variable domains of heavy-chain-only antibodies (VHH) (NANOBODY) bound to an ALFA-tag peptide. In embodiments, the DNA binder further comprising recombinant variable domains of heavy-chain-only antibodies (VHH) (NANOBODY) bound to an ALFA-tag peptide comprises an amino acid sequence of at least about 90% identity, or at least about 93% identity, or at least about 95% identity, or at least about 98% identity, or at least about 99% identity to SEQ ID NO: 886.

SEQ ID NO: 886: HoD4 TALE fused to the recombinant variable
domains of heavy-chain-only antibodies (VHH)
(NANOBODY) bound to an ALFA-tag peptide (NbALFA)
ATGGCGCCTAAGAAGAAGCGCAAGGTGGGCGGAGTTGACCTCCGGACTTTGGGTTACTCCCAACAGCAACAAGAGAA
GATTAAACCTAAGGTGCGCTCAACAGTGGCGCAGCATCATGAGGCACTGGTTGGTCACGGGTTCACACATGCCCATA
TAGTAGCACTTTCACAACATCCCGCTGCACTCGGAACGGTTGCAGTAAAGTACCAAGACATGATTGCCGCATTGCCG
GAAGCAACTCATGAGGCCATTGTCGGAGTAGGCAAATACCACGGTGCTAGAGCTCTCGAAGCACTCCTCACTGTGGC
TGGCGAATTGAGGGGGCCCCCCCTTCAACTTGATACTGGACAGCTGTTGAAGATAGCGAAACGAGGGGGAGTAACCG
CAGTCGAGGCCGTCCACGCGTGGCGAAATGCATTGACGGGAGCGCCTCTCAACCTTACTCCAGATCAGGTTGTGGCC
ATAGCGAGCAACATCGGAGGTAAGCAAGCACTGGAAACTGTTCAACGATTGCTCCCAGTACTCTGCCAAGACCACGG
ATTGACCCCAGAACAGGTTGTGGCTATTGCCAGCAACGGCGGCGGTAAACAGGCTCTGGAAACTGTGCAGCGACTCT
TGCCGGTGTTGTGCCAAGCTCATGGCCTTACGCCAGACCAGGTTGTTGCTATCGCTAGCAACATCGGAGGCAAACAG
GCACTTGAAACGGTCCAACGCCTCTTGCCAGTATTGTGCCAGGCACATGGCCTCACCCCCGCCCAGGTTGTGGCCAT
CGCAAACAATAACGGGGGCAAGCAGGCTTTGGAGACAGTCCAACGATTGTTGCCGGTACTTTGTCAGGACCACGGGC
TGACCCCAGATCAAGTCGTAGCAATCGCTAGCAACATCGGCGGGAAGCAGGCCCTGGAAACGGTGCAGAGGCTCCTC
CCAGTGCTCTGCCAAGATCATGGCCTCACCCCAGAGCAAGTGGTCGCAATCGCAAACAATAACGGTGGCAAGCAAGC
CCTCGAAACTGTGCAAAGGTTGCTGCCAGTTCTCTGTCAAGCCCACGGTTTGACACCGGATCAGGTAGTGGCTATCG
CTAGCAACGGCGGTGGCAAGCAAGCTCTCGAGACAGTCCAGAGGCTTTTGCCAGTACTGTGCCAAGCGCACGGTCTC
ACTCCTGCGCAAGTTGTTGCTATAGCAAACAATAACGGTGGAAAGCAAGCTCTTGAGACTGTGCAGCGCCTGTTGCC
CGTCCTCTGTCAGGACCACGGTTTGACTCCAGACCAAGTTGTCGCCATTGCAAGCAACGGCGGTGGACGCCCTGCAC
TGGAGTCCATCGTCGCACAACTGTCTAGGCCGGACCCCGCCCTGGCTGCCCTTACTAATGATCACTTGGTTGCATTG
GCGTGTCTGGGCGGTCGGCCAGCGCTGGACGCAGTCAAGAAGGGACTCCCCCACGCCCCTGCACTTATAAAACGCAC
TAACAGGAGAATACCAGAACGGACGAGTCATCGAGTCGCTGGAGGGGGCGGTAGCGAAGTCCAGCTCCAAGAAAGCG
GCGGGGGCCTCGTGCAGCCAGGCGGGTCCCTGAGGCTGAGCTGTACCGCATCTGGAGTCACCATCTCTGCCCTTAAC
GCAATGGCTATGGGGTGGTATCGGCAGGCGCCCGGCGAGAGGAGAGTCATGGTAGCAGCCGTTAGCGAAAGGGGGAA
TGCGATGTACCGAGAGAGTGTTCAGGGACGATTTACTGTCACTCGGGATTTTACGAACAAAATGGTTTCATTGCAAA
TGGACAATTTGAAACCAGAGGACACCGCTGTTTACTACTGTCACGTCCTGGAAGATCGAGTAGATAGCTTCCATGAC
TATTGGGGCCAAGGAACACAAGTGACTGTCAGCTCC

In embodiments, the helper enzyme of the present disclosure further comprises a linker inserted after the G66 residue relative to SEQ ID NO: 1 of MLT with R333K_N335A_R336K_D416N substitutions relative to SEQ ID NO: 1. In embodiments, the helper enzyme of the present disclosure further comprises an ALFA tag inserted after the G66 residue relative to SEQ ID NO: 1 of MLT with R333K_N335A_R336K_D416N substitutions relative to SEQ ID NO: 1.

In embodiments, the helper enzyme of the present disclosure further comprises a linker inserted immediately after the G66 residue relative to SEQ ID NO: 1 of MLT with R333K_N335A_R336K_D416N substitutions relative to SEQ ID NO: 1. In embodiments, the helper enzyme of the present disclosure further comprises an ALFA tag inserted immediately after the G66 residue relative to SEQ ID NO: 1 of MLT with R333K_N335A_R336K_D416N substitutions relative to SEQ ID NO: 1.

In embodiments, the helper enzyme comprises an amino acid sequence of at least about 90% identity, or at least about 93% identity, or at least about 95% identity, or at least about 98% identity, or at least about 99% identity to SEQ ID NO: 887.

SEQ ID NO: 887: ALFA tag inserted after the G66 residue
relative to SEQ ID NO: 1 of MLT with mutation:
R333K_N335A_R336K_D416N substitutions relative to SEQ ID NO: 1
ATGCCGAAAAAAAAACGAAAGGTGTACCCCTACGATGTACCGGACTATGCAGGAAGCGCCCAGCACAGCGACTACCC
CGACGACGAGTTCAGAGCCGATAAGCTGAGTAACTACAGCTGCGACAGCGACCTGGAAAACGCCAGCACATCCGACG
AGGACAGCTCTGACGACGAGGTGATGGTGCGGCCCAGAACCCTGAGACGGAGAAGAATCAGCAGCTCTAGCAGCGAC
TCTGAATCCGACATCGAGGGCTCCAGACTGGAAGAGGAACTGAGAAGAAGGCTCACAGAAGGCCGGGAAGAGTGGAG
CCACGTGGACAACCCTCCTGTTCTGGAAGATTTTCTGGGCCATCAGGGCCTGAACACCGACGCCGTGATCAACAACA
TCGAGGATGCCGTGAAGCTGTTCATAGGAGATGATTTCTTTGAGTTCCTGGTCGAGGAATCCAACCGCTATTACAAC
CAGAATAGAAACAACTTCAAGCTGAGCAAGAAAAGCCTGAAGTGGAAGGACATCACCCCTCAGGAGATGAAAAAGTT
CCTGGGACTGATCGTTCTGATGGGACAGGTGCGGAAGGACAGAAGGGATGATTACTGGACAACCGAACCTTGGACCG
AGACCCCTTACTTTGGCAAGACCATGACCAGAGACAGATTCAGACAGATCTGGAAAGCCTGGCACTTCAACAACAAT
GCTGATATCGTGAACGAGTCTGATAGACTGTGTAAAGTGCGGCCAGTGTTGGATTACTTCGTGCCTAAGTTCATCAA
CATCTATAAGCCTCACCAGCAGCTGAGCCTGGATGAAGGCATCGTGCCCTGGCGGGGCAGACTGTTCTTCAGAGTGT
ACAATGCTGGCAAGATCGTCAAATACGGCATCCTGGTGCGCCTTCTGTGCGAGAGCGATACAGGCTACATCTGTAAT
ATGGAAATCTACTGCGGCGAGGGCAAAAGACTGCTGGAAACCATCCAGACCGTCGTTTCCCCTTATACCGACAGCTG
GTACCACATCTACATGGACAACTACTACAATTCTGTGGCCAACTGCGAGGCCCTGATGAAGAACAAGTTTAGAATCT
GCGGCACAATCAAGAAAGCCAAGGGCATCCCTAAGGACTTCCAGACCATCTCTCTGAAGAAGGGCGAAACCAAGTTC
ATCAGAAAGAACGACATCCTGCTCCAAGTGTGGCAGTCCAAGAAACCCGTGTACCTGATCAGCAGCATCCATAGCGC
CGAGATGGAAGAAAGCCAGAACATCGACAGAACAAGCAAGAAGAAGATCGTGAAGCCCAATGCTCTGATCGACTACA
ACAAGCACATGAAAGGCGTGGACCGGGCCAACCAGTACCTGTCTTATTACTCTATCCTGAGAAGAACAGTGAAATGG
ACCAAGAGACTGGCCATGTACATGATCAATTGCGCCCTGTTCAACAGCTACGCCGTGTACAAGTCCGTGCGACAAAG
AAAAATGGGATTCAAGATGTTCCTGAAGCAGACAGCCATCCACTGGCTGACAGACGACATTCCTGAGGACATGGACA
TTGTGCCAGATCTGCAACCTGTGCCCAGCACCTCTGGTATGAGAGCTAAGCCTCCCACCAGCGATCCTCCATGTAGA
CTGAGCATGGACATGCGGAAGCACACCCTGCAGGCCATCGTCGGCAGCGGCAAGAAGAAGAACATCCTTAGACGGTG
CAGGGTGTGCAGCGTGCACAAGCTGCGGAGCGAGACTCGGTACATGTGCAAGTTTTGCAACATTCCCCTGCACAAGG
GAGCCTGCTTCGAGAAGTACCACACCCTGAAGAATTAC

In embodiments, the DNA binder of the present disclosure further comprises recombinant variable domains of heavy-chain-only antibodies (VHH) (NANOBODY) bound bound to an ALFA-tag peptide. In embodiments, the DNA binder further comprising recombinant variable domains of heavy-chain-only antibodies (VHH) (NANOBODY) bound to an ALFA-tag peptide comprises an amino acid sequence of at least about 90% identity, or at least about 93% identity, or at least about 95% identity, or at least about 98% identity, or at least about 99% identity to SEQ ID NO: 888. In embodiments, the DNA binder further comprising recombinant variable domains of heavy-chain-only antibodies (VHH) (NANOBODY) bound to an ALFA-tag peptide comprises an amino acid sequence of at least about 90% identity, or at least about 93% identity, or at least about 95% identity, or at least about 98% identity, or at least about 99% identity to SEQ ID NO: 889.

SEQ ID NO: 888: Ch6L left TALE fused to the recombinant variable
domains of heavy-chain-only antibodies (VHH)
(NANOBODY) bound to an ALFA-tag peptide (NbALFA)
ATGGCGCCTAAGAAGAAGCGCAAGGTGGGCGGAGTTGACCTCCGGACTTTGGGTTACTCCCAACAGCAACAAGAGAA
GATTAAACCTAAGGTGCGCTCAACAGTGGCGCAGCATCATGAGGCACTGGTTGGTCACGGGTTCACACATGCCCATA
TAGTAGCACTTTCACAACATCCCGCTGCACTCGGAACGGTTGCAGTAAAGTACCAAGACATGATTGCCGCATTGCCG
GAAGCAACTCATGAGGCCATTGTCGGAGTAGGCAAATACCACGGTGCTAGAGCTCTCGAAGCACTCCTCACTGTGGC
TGGCGAATTGAGGGGGCCCCCCCTTCAACTTGATACTGGACAGCTGTTGAAGATAGCGAAACGAGGGGGAGTAACCG
CAGTCGAGGCCGTCCACGCGTGGCGAAATGCATTGACGGGAGCGCCTCTCAACCTTACGCCGGATCAAGTCGTGGCG
ATAGCTAGTAACGGAGGTGGTAAACAGGCGCTGGAGACGGTACAGCGCTTGCTGCCAGTGCTCTGCCAAGATCACGG
CCTTACGCCAGAGCAAGTGGTGGCTATTGCGAATAATAACGGTGGAAAACAGGCGTTGGAAACAGTCCAAAGACTGC
TCCCGGTGTTGTGCCAAGCTCATGGACTGACGCCCGATCAAGTAGTAGCTATCGCAAGCAACGGTGGGGGGAAACAG
GCACTGGAGACTGTCCAGAGGCTCCTCCCTGTGTTGTGCCAAGCCCATGGGCTTACTCCAGCTCAGGTTGTAGCGAT
CGCCAGCAACATTGGAGGAAAGCAGGCCCTGGAAACTGTTCAACGCCTCCTCCCAGTATTGTGCCAAGATCACGGAC
TTACCCCGGATCAAGTCGTCGCAATCGCGTCCCACGATGGCGGGAAACAGGCACTTGAGACCGTGCAACGACTGTTG
CCCGTGCTCTGCCAAGACCATGGCCTCACACCTGAGCAAGTAGTGGCGATTGCGTCTCACGATGGGGGCAAGCAGGC
GCTTGAGACCGTTCAGCGATTGCTTCCTGTCCTCTGCCAGGCCCACGGGTTGACACCTGATCAGGTGGTAGCCATTG
CGTCTAACGGTGGAGGTAAGCAAGCACTCGAGACCGTTCAAAGGCTCTTGCCAGTATTGTGTCAGGCTCATGGTCTT
ACGCCAGCCCAAGTTGTCGCCATCGCATCTCATGACGGTGGGAAGCAGGCACTTGAGACTGTACAAAGATTGCTCCC
AGTGTTGTGCCAGGACCACGGCTTGACGCCTGATCAAGTTGTAGCGATTGCTTCACATGATGGGGGCAGGCCTGCGT
TGGAGTCCATCGTCGCACAACTGTCTAGGCCGGACCCCGCCCTGGCTGCCCTTACTAATGATCACTTGGTTGCATTG
GCGTGTCTGGGCGGTCGGCCAGCGCTGGACGCAGTCAAGAAGGGACTCCCCCACGCCCCTGCACTTATAAAACGCAC
TAACAGGAGAATACCAGAACGGACGAGTCATCGAGTCGCTGGAGGGGGCGGTAGCGAAGTCCAGCTCCAAGAAAGCG
GCGGGGGCCTCGTGCAGCCAGGCGGGTCCCTGAGGCTGAGCTGTACCGCATCTGGAGTCACCATCTCTGCCCTTAAC
GCAATGGCTATGGGGTGGTATCGGCAGGCGCCCGGCGAGAGGAGAGTCATGGTAGCAGCCGTTAGCGAAAGGGGGAA
TGCGATGTACCGAGAGAGTGTTCAGGGACGATTTACTGTCACTCGGGATTTTACGAACAAAATGGTTTCATTGCAAA
TGGACAATTTGAAACCAGAGGACACCGCTGTTTACTACTGTCACGTCCTGGAAGATCGAGTAGATAGCTTCCATGAC
TATTGGGGCCAAGGAACACAAGTGACTGTCAGCTCC
SEQ ID NO: 889: Ch6R right TALE fused to the recombinant variable
domains of heavy-chain-only antibodies (VHH)
(NANOBODY) bound to an ALFA-tag peptide (NbALFA)
ATGGCGCCTAAGAAGAAGCGCAAGGTGGGCGGAGTTGACCTCCGGACTTTGGGTTACTCCCAACAGCAACAAGAGAA
GATTAAACCTAAGGTGCGCTCAACAGTGGCGCAGCATCATGAGGCACTGGTTGGTCACGGGTTCACACATGCCCATA
TAGTAGCACTTTCACAACATCCCGCTGCACTCGGAACGGTTGCAGTAAAGTACCAAGACATGATTGCCGCATTGCCG
GAAGCAACTCATGAGGCCATTGTCGGAGTAGGCAAATACCACGGTGCTAGAGCTCTCGAAGCACTCCTCACTGTGGC
TGGCGAATTGAGGGGGCCCCCCCTTCAACTTGATACTGGACAGCTGTTGAAGATAGCGAAACGAGGGGGAGTAACCG
CAGTCGAGGCCGTCCACGCGTGGCGAAATGCATTGACGGGAGCGCCTCTCAACCTTACACCTGATCAAGTAGTTGCC
ATTGCGAGTCATGATGGTGGGAAACAGGCCTTGGAAACTGTCCAGCGCCTTCTGCCTGTGCTTTGTCAAGATCATGG
GTTGACACCCGAGCAAGTTGTAGCTATTGCGTCACACGACGGTGGTAAACAGGCGCTGGAAACTGTTCAGCGCTTGC
TCCCAGTCCTCTGTCAGGCCCATGGTCTCACCCCGGACCAGGTAGTAGCTATAGCTAGCAATATCGGAGGAAAGCAA
GCGTTGGAAACCGTGCAGCGGTTGCTCCCCGTCCTGTGTCAAGCACACGGCTTGACGCCGGCCCAGGTGGTAGCCAT
TGCGTCTAACATCGGCGGCAAGCAGGCACTCGAGACTGTGCAACGGCTCCTGCCGGTTCTGTGCCAAGATCACGGTC
TTACTCCTGATCAGGTCGTTGCGATTGCTAGTAATGGCGGTGGAAAACAGGCCCTGGAGACTGTCCAGCGGCTTCTC
CCTGTCCTGTGCCAGGACCATGGACTCACACCAGAACAGGTAGTTGCAATAGCCTCTAATATAGGTGGCAAGCAAGC
ACTCGAGACAGTCCAAAGACTTCTTCCCGTCCTTTGCCAGGCGCATGGTCTCACCCCTGATCAAGTTGTTGCGATAG
CGAGTAACATTGGAGGAAAACAGGCATTGGAAACGGTACAACGGCTTCTCCCGGTCTTGTGTCAAGCGCATGGCCTC
ACCCCGGCCCAAGTGGTCGCTATAGCATCCAATATTGGGGGTAAGCAAGCACTGGAAACGGTTCAGCGACTTTTGCC
TGTGCTGTGCCAAGACCACGGACTTACACCGGACCAAGTTGTCGCTATCGCCTCCAACGGGGGAGGACGGCCGGCCT
TGGAGTCCATCGTCGCACAACTGTCTAGGCCGGACCCCGCCCTGGCTGCCCTTACTAATGATCACTTGGTTGCATTG
GCGTGTCTGGGCGGTCGGCCAGCGCTGGACGCAGTCAAGAAGGGACTCCCCCACGCCCCTGCACTTATAAAACGCAC
TAACAGGAGAATACCAGAACGGACGAGTCATCGAGTCGCTGGAGGGGGCGGTAGCGAAGTCCAGCTCCAAGAAAGCG
GCGGGGGCCTCGTGCAGCCAGGCGGGTCCCTGAGGCTGAGCTGTACCGCATCTGGAGTCACCATCTCTGCCCTTAAC
GCAATGGCTATGGGGTGGTATCGGCAGGCGCCCGGCGAGAGGAGAGTCATGGTAGCAGCCGTTAGCGAAAGGGGGAA
TGCGATGTACCGAGAGAGTGTTCAGGGACGATTTACTGTCACTCGGGATTTTACGAACAAAATGGTTTCATTGCAAA
TGGACAATTTGAAACCAGAGGACACCGCTGTTTACTACTGTCACGTCCTGGAAGATCGAGTAGATAGCTTCCATGAC
TATTGGGGCCAAGGAACACAAGTGACTGTCAGCTCC

In embodiments, the helper enzyme comprises a deletion of about 30, or about 40, or about 50, or about 60, or about 70, or about 80, or about 90, or about 100 amino acids from an N-terminus or a C-terminus.

In embodiments, the helper enzyme comprises a deletion of about 30, or about 40, or about 50, or about 60, or about 70 amino acids from an N-terminus of the polypeptide having an amino acid sequence of SEQ ID NO: 1. In embodiments, the helper enzyme comprises a deletion at positions about 1-35, or about 1-45, or about 1-55, or about 1-65, or about 1-75, or about 1-85, or about 1-95, or about 1-105, or about 2-35, or about 2-45, or about 2-68 or positions corresponding thereto, wherein the positions are relative to SEQ ID NO: 1.

In embodiments, the helper enzyme comprises a deletion at positions about 2-35 (SEQ ID NO: 504), or about 2-45 (SEQ ID NO: 506), or about 2-68 (SEQ ID NO: 508), or positions corresponding thereto, wherein the positions are relative to SEQ ID NO: 1. In embodiments, the N terminal deletion yields reduced or ablated off-target effects of the enzyme compared to the enzyme without the N terminal deletion. In embodiments, the helper enzyme comprising the N terminal deletion has a sequence selected from SEQ ID NO: 504, 506, or 508, or a sequence having at least about 95%, or at least about 96%, at least about 97%, at least about 98%, at least about 99% identity thereto.

In embodiments, the helper enzyme has increased activity relative to an enzyme comprising an amino acid sequence of SEQ ID NO: 1, or functional equivalent thereof.

In embodiments, the helper enzyme is excision positive. In embodiments, the helper enzyme is integration deficient. In embodiments, the helper enzyme has decreased integration activity relative to a helper enzyme comprising an amino acid sequence of SEQ ID NO: 1, or functional equivalent thereof. In embodiments, the helper enzyme has increased excision activity relative to a helper enzyme comprising an amino acid sequence of SEQ ID NO: 1, or functional equivalent thereof.

In embodiments, the helper enzyme of the present disclosure comprises a substitution selected from TABLE 1, TABLE 1A, TABLE 2 and/or TABLE 2A, a deletion at positions about 2-35 (SEQ ID NO: 504), or about 2-45 (SEQ ID NO: 506), or about 2-68 (SEQ ID NO: 508), or substitutions and positions corresponding thereto relative to SEQ ID NO: 1, and a DNA binding domain selected from TABLE 8-17.

In embodiments, the enzyme is MLT. In embodiments, the helper enzyme comprising an N terminal deletion is further fused to a DNA binding domain. In embodiments, the DNA binder comprises TALEs, ZnF, and/or dCas (TABLES 8-17). In embodiments, the helper enzyme further comprises a targeting element. In embodiments, the helper enzyme is capable of inserting a donor comprising a transgene in a genomic safe harbor site (GSHS) and/or wherein the targeting element is suitable for directing the helper enzyme to a GSHS.

In embodiments, the binding of a GSHS of a nucleic acid molecule in a mammalian cell is with high target specificity, relative to a control. In embodiments, the control is a composition comprising a helper enzyme comprising an amino acid sequence of SEQ ID NO: 1 or a nucleic acid comprising one or more of the Exc+Int− mutations and DNA binders in TABLES 1-17 and 19-20 or a codon-optimized forms thereof.

In embodiments, the targeting element is able to direct a transposition machinery to the GSHS of a nucleic acid molecule in a mammalian cell. In embodiments, the targeting element is able to direct a transposition machinery to a GSHS or or intron 1 of a disease gene (e.g., HPRT, CFTR) of a nucleic acid molecule in a mammalian cell. In embodiments, the GSHS is in an open chromatin location in a chromosome.

In embodiments, the GSHS is selected from the human Rosa26 locus, adeno-associated virus site 1 (AAVS1), chromosome 4 GSHS, chromosome 22 GSHS, chemokine (C—C motif) receptor 5 (CCR5) gene, HIV-1 coreceptor. In embodiments, the GSHS is a located at the human Rosa26 locus. In embodiments, the GSHS is an adeno-associated virus site 1 (AAVS1). In embodiments, the GSHS is located on human chromosome 2, 3, 4, 6, 10, 11, 17, 19, 22, or X. In embodiments, the DNA binding element that directs the transpoase to GSHS is selected from TABLES 3-17 and TABLES 19-20. In embodiments, the guide RNA for targeting human genomic safe harbor sites using any of the gRNA based targeting elements, is selected from GUIDE 44, GUIDE 45-C, GUIDE 46-C, SPG GUIDE1-C, SPG GUIDE2-C, SPG GUIDE3-C, SPG GUIDE4-C, SPG GUIDE5-C, SPG GUIDE6-C, SPG GUIDE7-C, SPG GUIDE8, SPG GUIDE9, SPG GUIDE10, SPG GUIDE11, SPG GUIDE12, SPG GUIDE13, SPG GUIDE14, GUIDE N1, GUIDE N2, GUIDE N3-C, GUIDE 012, GUIDE 013, GUIDE 014-C, AAV GUIDE 12, AAV GUIDE 13c, AAV GUIDE 14, AAV GUIDE 14c, AAV GUIDE 15, AAV GUIDE 16, AAV GUIDE 17, AAV GUIDE 18, AAV GUIDE 19, AAV GUIDE 20, AAV GUIDE 21, AAV GUIDE 22, AAV GUIDE 23, AAV GUIDE 24, AAV GUIDE 25, AAV GUIDE 26, AAV GUIDE 27, AAV GUIDE 28, AAV GUIDE 29, AAV GUIDE 30c, AAV GUIDE 31, AAV GUIDE 32c, AAV GUIDE 33c, AAV GUIDE 34, AAV GUIDE 35, Guide C4-1, Guide C4-2, Guide C4-3, Guide C4-4, Guide C4-5, Guide C4-6, Guide C4-7, Guide C4-8, Guide C4-9, Guide C4-10, Guide C4-11, Guide C4-12, Guide C4-13, Guide C4-14, Guide C4-15, Guide C4-16, Guide C4-17, Guide C4-18, Guide C4-19, Guide C4-20, Guide C4A1, Guide C4A2, Guide C4A3, Guide C4A4, Guide C4A5, Guide C4A6, Guide C4A7, Guide C4A8, Guide C4A9, Guide C4A10, Guide C4A11, Guide C4A12, Guide C4A13, Guide C4A14, Guide C4A15, Guide C4A16, Guide C4A17, Guide C4A18, Guide C4A19, Guide C4A20, Guide C22-1, Guide C22-2, Guide C22-3, Guide C22-4, Guide C22-5, Guide C22-6, Guide C22-7, Guide C22-8, Guide C22-9, Guide C22-10, Guide C22-11, Guide C22-12, Guide C22-13, Guide C22-14, Guide C22-15, Guide C22-16, Guide C22-17, Guide C22-18, Guide C22-19, Guide C22-20, Guide C22A1, Guide C22A2, Guide C22A3, Guide C22A4, Guide C22A5, Guide C22A6, Guide C22A7, Guide C22A8, Guide C22A9, Guide C22A10, Guide C22A11, Guide C22A12, Guide C22A13, Guide C22A14, Guide C22A15, Guide C22A16, Guide C22A17, Guide C22A18, Guide C22A19, Guide C22A20, Guide CX-1, Guide CX-2, Guide CX-3, Guide CX-4, Guide CX-5, Guide CX-6, Guide CX-7, Guide CX-8, Guide CX-9, Guide CX-10, Guide CX-11, Guide CX-12, Guide CX-13, Guide CX-14, Guide CX-15, Guide CX-16, Guide CX-17, Guide CX-18, Guide CX-19, Guide CX-20, TALES: R1, R2, R3, R4, R5, R6, R7, R8, R9, R10, R11, R12, R13, R14, AAV1c, AAV2c, AAV3c, AAV4c, AAV5c, AAV6, AAV7, AAV8, AAV9, AAV10, AAV11, AAV12, AAV13c, AAV14c, AAV15c, AAV16c, AAV17c, TALE4-R001, TALE4-R002, TALE4-F003, TALE4-F004, TALE4-F005, TALE4-F006, TALE4-R007, TALE4-F008, TALE4-R009, TALE4-F010, TALE4-F011, TALE4-F012, TALE4-R013, TALE4-F014, TALE4-F015, TALE4-R016, TALE4-F017, TALE4-F018, TALE4-F019, TALE4-F020, TALE22F-R001, TALE22-F002, TALE22-F003, TALE22-F004, TALE22-F005, TALE22-F006, TALE22-F007, TALE22-F008, TALE22-R009, TALE22-F010, TALE22-F001A, TALE22-F002A, TALE22-R03A, TALE22-F004A, TALE22-F005A, TALE22-F006A, TALE22-R007A, TALE22-R008A, TALE22-R009A, TALE22-F010A, TALE F002, TALE F003, TALE F004, TALE F007, TALE F008, TALE F009, TALE R002, TALE R003, TALE R004, TALE R005, TALE R007, TALE F020, TALE F021, TALE F030, TALE F031, TALE F034, TALE F036, TALE F037, TALE F040, TALE R022, TALE R033, TALE R035, TALE R038, TALE R039, Zinc Fingers: ZnF3a, ZnF5a, ZnF5b, ZnF5c, ZnF5d, ZnF5e, ZnF5f, ZnF5g, ZnF5h, ZnF12a, ZnF13a, ZnF13b, ZnF13c, ZnF11a, ZnF10a, ZnF12b, ZnF13b, ZnF14a, ZnF15a, ZnF16a, ZnF17a, ZnF18a, ZnF19a, ZnF20b, ZnF21b, ZnF22a, ZnF23a, ZnF24a, ZnF31F, ZnF32F, ZnF33F, ZnF34F, ZnF35F, ZnF36F, ZnF37R, ZnF38R, ZnF39R, ZnF1a, ZnF1b, ZnF2a, ZnF3a, ZnF3b, ZnF5aR, ZnF5bR, ZnF6aR, ZnF6bR, ZnF10F, ZnF11F, ZnF12F, ZnF13F, ZnF14R, ZnF15R, ZnF14R, ZnF15R, ZnF41F, ZnF42F, ZnF43F, ZnF44R, ZnF45R, ZnF46R, ZnF47R, ZnF48R.

In embodiments, the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TA-LER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

In embodiments, the GSHS comprises one or more TTAA integration sites. In embodiments, the targeting element directs the helper enzyme to either one or more nucleic acid binding sites that are upstream and/or downstream of the TTAA integration sites or to the TTAA integration sites. In embodiments, the targeting element directs the helper enzyme to one or more nucleic acid binding sites that are upstream and/or downstream of the TTAA integration sites and within about 5 to about 30 base pairs of the TTAA integration sites or within about 15 to about 19 base pairs of the TTAA integration sites. In embodiments, the targeting element directs the helper enzyme to two nucleic acid binding sites of the TTAA integration sites, wherein a first site is upstream of TTAA and within about 5 to about 30 base pairs or about 15 to about 19 base pairs of the TTAA and a second site is downstream of TTAA and within about 5 to about 30 base pairs or about 15 to about 19 base pairs of the TTAA.

In embodiments, the targeting element is or comprises one or more of a Cas enzyme, which is optionally catalytically inactive and which is optionally associated with a guide RNA (gRNA), transcription activator-like effector (TALE) DNA binding domain (DBD), Zinc finger, catalytically inactive transcription factor, catalytically inactive nickase, a transcriptional activator, a transcriptional repressor, a recombinase, a DNA methyltransferase, a histone methyltransferase, a paternally expressed gene 10 (PEG10), and a transposon-encoded polypeptide D (TnsD) or a variant thereof. In embodiments, the targeting element comprises a TALE DBD. In embodiments, the TALE DBD comprises one or more repeat sequences. In embodiments, the TALE DBD comprises about 14, or about 15, or about, 16, or about 17, or about 18, or about 18.5 repeat sequences. In embodiments, the repeat sequences each independently comprises about 33 or 34 amino acids. In embodiments, the repeat sequences each independently comprises a repeat variable di-residue (RVD) at residue 12 or 13 of the 33 or 34 amino acids, respectively. In embodiments, the RVD recognizes one base pair in a target nucleic acid sequence. In embodiments, the RVD recognizes a C residue in the target nucleic acid sequence and is selected from HD, N (gap), HA, ND, and HI. In embodiments, the RVD recognizes a G residue in the target nucleic acid sequence and is selected from NN, NH, NK, HN, and NA. In embodiments, the RVD recognizes an A residue in the target nucleic acid sequence and is selected from NI and NS. In embodiments, the RVD recognizes a T residue in the target nucleic acid sequence and is selected from NG, HG, H (gap), and IG.

In embodiments, the TALE DBD targets one or more of GSHS sites selected from TABLES 8-12 and TABLE 20.

In embodiments, the TALE DBD comprises one or more of RVD se-lected from TABLES 8-12 and TABLE 20, or variants thereof comprising about 1, about 2, about 3, about 4, about 5, about 6, about 7, about 8, about 9, about 10, about 11, about 12, about 13, about 14, about 15, about 16, about 17, about 18, about 19, or about 20 mutations.

In embodiments, the gRNA targets one or more of GSHS sites selected from TABLES 3-7 and TABLE 19. In embodiments, the targeting element comprises a Cas9 enzyme associated with a gRNA. In embodiments, the Cas9 enzyme associated with a gRNA comprises a catalytically inactive dCas9 associated with a gRNA.

In embodiments, the targeting element is or comprises a Cas9 enzyme associated with a gRNA or a CasX enzyme associated with a gRNA.

In embodiments, the target element is or comprises a CasX enzyme associated with a gRNA, optionally wherein the catalytic inactive dCasX

In embodiments, the catalytically inactive dCas9 comprises at least about 90%, or at least about 95%, or at least about 96%, or at least about 97%, or at least about 98%, or at least about 99% identity to an amino acid sequence of SEQ ID NO: 6 or a nucleic acid comprising a nucleotide sequence of SEQ ID NO: 5 or a codon-optimized form thereof.

```
SEQ ID NO: 5: nucleotide sequence of dead Cas9 DNA BINDING protein (5004 bp)
      1 ATGGACAAGA AGTACTCCAT TGGGCTCGCT ATCGGCACAA ACAGCGTCGG CTGGGCCGTC
    61 ATTACGGACG AGTACAAGGT GCCGAGCAAA AAATTCAAAG TTCTGGGCAA TACCGATCGC
121 CACAGCATAA AGAAGAACCT CATTGGCGCC CTCCTGTTCG ACTCCGGGGA GACGGCCGAA
  181 GCCACGCGGC TCAAAAGAAC AGCACGGCGC AGATATACCC GCAGAAAGAA TCGGATCTGC
  241 TACCTGCAGG AGATCTTTAG TAATGAGATG GCTAAGGTGG ATGACTCTTT CTTCCATAGG
  301 CTGGAGGAGT CCTTTTTGGT GGAGGAGGAT AAAAAGCACG AGCGCCACCC AATCTTTGGC
  361 AATATCGTGG ACGAGGTGGC GTACCATGAA AAGTACCCAA CCATATATCA TCTGAGGAAG
  421 AAGCTTGTAG ACAGTACTGA TAAGGCTGAC TTGCGGTTGA TCTATCTCGC GCTGGCGCAT
  481 ATGATCAAAT TTCGGGGACA CTTCCTCATC GAGGGGGACC TGAACCCAGA CAACAGCGAT
  541 GTCGACAAAC TCTTTATCCA ACTGGTTCAG ACTTACAATC AGCTTTTCGA AGAGAACCCG
  601 ATCAACGCAT CCGGAGTTGA CGCCAAAGCA ATCCTGAGCG CTAGGCTGTC CAAATCCCGG
  661 CGGCTCGAAA ACCTCATCGC ACAGCTCCCT GGGGAGAAGA AGAACGGCCT GTTTGGTAAT
  721 CTTATCGCCC TGTCACTCGG GCTGACCCCC AACTTTAAAT CTAACTTCGA CCTGGCCGAA
  781 GATGCCAAGC TTCAACTGAG CAAAGACACC TACGATGATG ATCTCGACAA TCTGCTGGCC
  841 CAGATCGGCG ACCAGTACGC AGACCTTTTT TTGGCGGCAA AGAACCTGTC AGACGCCATT
  901 CTGCTGAGTG ATATTCTGCG AGTGAACACG GAGATCACCA AAGCTCCGCT GAGCGCTAGT
  961 ATGATCAAGC GCTATGATGA GCACCACCAA GACTTGACTT TGCTGAAGGC CCTTGTCAGA
1021 CAGCAACTGC CTGAGAAGTA CAAGGAAATT TTCTTCGATC AGTCTAAAAA TGGCTACGCC
1081 GGATACATTG ACGGCGGAGC AAGCCAGGAG GAATTTTACA AATTTATTAA GCCCATCTTG
1141 GAAAAAATGG ACGGCACCGA GGAGCTGCTG GTAAAGCTTA ACAGAGAAGA TCTGTTGCGC
1201 AAACAGCGCA CTTTCGACAA TGGAAGCATC CCCCACCAGA TTCACCTGGG CGAACTGCAC
1261 GCTATCCTCA GGCGGCAAGA GGATTTCTAC CCCTTTTTGA AAGATAACAG GGAAAAGATT
1321 GAGAAAATCC TCACATTTCG GATACCCTAC TATGTAGGCC CCCTCGCCCG GGGAAATTCC
1381 AGATTCGCGT GGATGACTCG CAAATCAGAA GAGACCATCA CTCCCTGGAA CTTCGAGGAA
1441 GTCGTGGATA AGGGGGCCTC TGCCCAGTCC TTCATCGAAA GGATGACTAA CTTTGATAAA
1501 AATCTGCCTA ACGAAAAGGT GCTTCCTAAA CACTCTCTGC TGTACGAGTA CTTCACAGTT
1561 TATAACGAGC TCACCAAGGT CAAATACGTC ACAGAAGGGA TGAGAAAGCC AGCATTCCTG
1621 TCTGGAGAGC AGAAGAAAGC TATCGTGGAC CTCCTCTTCA AGACGAACCG GAAAGTTACC
1681 GTGAAACAGC TCAAAGAAGA CTATTTCAAA AAGATTGAAT GTTTCGACTC TGTTGAAATC
1741 AGCGGAGTGG AGGATCGCTT CAACGCATCC CTGGGAACGT ATCACGATCT CCTGAAAATC
1801 ATTAAAGACA AGGACTTCCT GGACAATGAG GAGAACGAGG ACATTCTTGA GGACATTGTC
1861 CTCACCCTTA CGTTGTTTGA AGATAGGGAG ATGATTGAAG AACGCTTGAA AACTTACGCT
1921 CATCTCTTCG ACGACAAAGT CATGAAACAG CTCAAGAGGC GCCGATATAC AGGATGGGGG
1981 CGGCTGTCAA GAAAACTGAT CAATGGGATC CGAGACAAGC AGAGTGGAAA GACAATCCTG
2041 GATTTTCTTA AGTCCGATGG ATTTGCCAAC CGGAACTTCA TGCAGTTGAT CCATGATGAC
2101 TCTCTCACCT TTAAGGAGGA CATCCAGAAA GCACAAGTTT CTGGCCAGGG GGACAGTCTT
2161 CACGAGCACA TCGCTAATCT TGCAGGTAGC CCAGCTATCA AAAAGGGAAT ACTGCAGACC
2221 GTTAAGGTCG TGGATGAACT CGTCAAAGTA ATGGGAAGGC ATAAGCCCGA GAATATCGTT
2281 ATCGAGATGG CCCGAGAGAA CCAAACTACC CAGAAGGGAC AGAAGAACAG TAGGGAAAGG
2341 ATGAAGAGGA TTGAAGAGGG TATAAAAGAA CTGGGGTCCC AAATCCTTAA GGAACACCCA
2401 GTTGAAAACA CCCAGCTTCA GAATGAGAAG CTCTACCTGT ACTACCTGCA GAACGGCAGG
2461 GACATGTACG TGGATCAGGA ACTGGACATC AATCGGCTCT CCGACTACGA CGTGGCTGCT
2521 ATCGTGCCCC AGTCTTTTCT CAAAGATGAT TCTATTGATA ATAAAGTGTT GACAAGATCC
2581 GATAAAGCTA GAGGGAAGAG TGATAACGTC CCCTCAGAAG AAGTTGTCAA GAAAATGAAA
2641 AATTATTGGC GGCAGCTGCT GAACGCCAAA CTGATCACAC AACGGAAGTT CGATAATCTG
2701 ACTAAGGCTG AACGAGGTGG CCTGTCTGAG TTGGATAAAG CCGGCTTCAT CAAAAGGCAG
2761 CTTGTTGAGA CACGCCAGAT CACCAAGCAC GTGGCCCAAA TTCTCGATTC ACGCATGAAC
2821 ACCAAGTACG ATGAAAATGA CAAACTGATT CGAGAGGTGA AAGTTATTAC TCTGAAGTCT
2881 AAGCTGGTCT CAGATTTCAG AAAGGACTTT CAGTTTTATA AGGTGAGAGA GATCAACAAT
2941 TACCACCATG CGCATGATGC CTACCTGAAT GCAGTGGTAG GCACTGCACT TATCAAAAAA
3001 TATCCCAAGC TTGAATCTGA ATTTGTTTAC GGAGACTATA AAGTGTACGA TGTTAGGAAA
3061 ATGATCGCAA AGTCTGAGCA GGAAATAGGC AAGGCCACCG CTAAGTACTT CTTTTACAGC
3121 AATATTATGA ATTTTTTCAA GACCGAGATT ACACTGGCCA ATGGAGAGAT TCGGAAGCGA
3181 CCACTTATCG AAACAAACGG AGAAACAGGA GAAATCGTGT GGGACAAGGG TAGGGATTTC
3241 GCGACAGTCC GGAAGGTCCT GTCCATGCCG CAGGTGAACA TCGTTAAAAA GACCGAAGTA
3301 CAGACCGGAG GCTTCTCCAA GGAAAGTATC CTCCCGAAAA GGAACAGCGA CAAGCTGATC
3361 GCACGCAAAA AAGATTGGGA CCCCAAGAAA TACGGCGGAT TCGATTCTCC TACAGTCGCT
3421 TACAGTGTAC TGGTTGTGGC CAAAGTGGAG AAAGGGAAGT CTAAAAAACT CAAAAGCGTC
3481 AAGGAACTGC TGGGCATCAC AATCATGGAG CGATCAAGCT TCGAAAAAAA CCCCATCGAC
3541 TTTCTGGAGG CGAAAGGATA TAAAGAGGTC AAAAAAGACC TCATCATTAA GCTTCCCAAG
3601 TACTCTCTCT TTGAGCTTGA AAACGGCCGG AAACGAATGC TCGCTAGTGC GGGCGAGCTG
3661 CAGAAAGGTA ACGAGCTGGC ACTGCCCTCT AAATACGTTA ATTTCTTGTA TCTGGCCAGC
3721 CACTATGAAA AGCTCAAAGG GTCTCCCGAA GATAATGAGC AGAAGCAGCT GTTCGTGGAA
3781 CAACACAAAC ACTACCTTGA TGAGATCATC GAGCAAATAA GCGAATTCTC CAAAAGAGTG
3841 ATCCTCGCCG ACGCTAACCT CGATAAGGTG CTTTCTGCTT ACAATAAGCA CAGGGATAAG
3901 CCCATCAGGG AGCAGGCAGA AAACATTATC CACTTGTTTA CTCTGACCAA CTTGGGCGCG
3961 CCTGCAGCCT TCAAGTACTT CGACACCACC ATAGACAGAA AGCGGTACAC CTCTACAAAG
4021 GAGGTCCTGG ACGCCACACT GATTCATCAG TCAATTACGG GGCTCTATGA AACAAGAATC
4081 GACCTCTCTC AGCTCGGTGG AGAC
SEQ ID NO: 6: amino acid sequence of dead Cas9 DNA BINDING protein
(1368 amino acids)
      1 MDKKYSIGLA IGTNSVGWAV ITDEYKVPSK KFKVLGNTDR HSIKKNLIGA LLFDSGETAE
    61 ATRLKRTARR RYTRRKNRIC YLQEIFSNEM AKVDDSFFHR LEESELVEED KKHERHPIFG
  121 NIVDEVAYHE KYPTIYHLRK KLVDSTDKAD LRLIYLALAH MIKERGHFLI EGDLNPDNSD
  181 VDKLFIQLVQ TYNQLFEENP INASGVDAKA ILSARLSKSR RLENLIAQLP GEKKNGLFGN
  241 LIALSLGLTP NEKSNEDLAE DAKLQLSKDT YDDDLDNLLA QIGDQYADLE LAAKNLSDAI
  301 LLSDILRVNT EITKAPLSAS MIKRYDEHHQ DLTLLKALVR QQLPEKYKEI FFDQSKNGYA
  361 GYIDGGASQE EFYKFIKPIL EKMDGTEELL VKLNREDLLR KQRTFDNGSI PHQIHLGELH
  421 AILRRQEDFY PFLKDNREKI EKILTFRIPY YVGPLARGNS RFAWMTRKSE ETITPWNFEE
  481 VVDKGASAQS FIERMTNEDK NLPNEKVLPK HSLLYEYFTV YNELTKVKYV TEGMRKPAFL
  541 SGEQKKAIVD LLFKTNRKVT VKQLKEDYFK KIECFDSVEI SGVEDRENAS LGTYHDLLKI
  601 IKDKDELDNE ENEDILEDIV LTLTLFEDRE MIEERLKTYA HLEDDKVMKQ LKRRRYTGWG
  661 RLSRKLINGI RDKQSGKTIL DELKSDGFAN RNFMQLIHDD SLTFKEDIQK AQVSGQGDSL
  721 HEHIANLAGS PAIKKGILQT VKVVDELVKV MGRHKPENIV IEMARENQTT QKGQKNSRER
  781 MKRIEEGIKE LGSQILKEHP VENTQLQNEK LYLYYLQNGR DMYVDQELDI NRLSDYDVAA
  841 IVPQSFLKDD SIDNKVLTRS DKARGKSDNV PSEEVVKKMK NYWRQLLNAK LITQRKEDNL
  901 TKAERGGLSE LDKAGFIKRQ LVETRQITKH VAQILDSRMN TKYDENDKLI REVKVITLKS
  961 KLVSDERKDF QFYKVREINN YHHAHDAYLN AVVGTALIKK YPKLESEFVY GDYKVYDVRK
1021 MIAKSEQEIG KATAKYFFYS NIMNFFKTEI TLANGEIRKR PLIETNGETG EIVWDKGRDE
1081 ATVRKVLSMP QVNIVKKTEV QTGGFSKESI LPKRNSDKLI ARKKDWDPKK YGGEDSPTVA
1141 YSVLVVAKVE KGKSKKLKSV KELLGITIME RSSFEKNPID FLEAKGYKEV KKDLIIKLPK
1201 YSLFELENGR KRMLASAGEL QKGNELALPS KYVNFLYLAS HYEKLKGSPE DNEQKQLFVE
1261 QHKHYLDEII EQISEFSKRV ILADANLDKV LSAYNKHRDK PIREQAENII HLFTLTNLGA
1321 PAAFKYFDTT IDRKRYTSTK EVLDATLIHQ SITGLYETRI DLSQLGGD
```

In embodiments, the targeting element comprises a Cas12 enzyme associated with a gRNA. In embodiments, the targeting element comprises a catalytically inactive Cas12 associated with a gRNA, optionally wherein the catalytically inactive Cas12 is dCas12j, dCas12a or a small DCasX. In embodiments, the targeting element comprises a TnsC, TnsB, TnsA, TniQ, Cas6, Cas7, Cas8 enzyme associated with a gRNA.

In embodiments, the guide RNA is selected from TABLES 3-7 and TABLE 19, or variants thereof comprising about 1, about 2, about 3, about 4, about 5, about 6, about 7, about 8, about 9, about 10, about 11, about 12, about 13, about 14, about 15, about 16, about 17, about 18, about 19, or about 20 mutations. In embodiments, the guide RNA targets one or more sites selected from TABLES 3-7 and TABLE 19. In embodiments, the zinc finger comprises one of the sequences selected from TABLES 13-17, or variants thereof comprising about 99, about 98, about 97, about 95, about 94, about 93, about 92, about 91, about 90, about 89, about 88, about 87, about 86, about 85, about 84, about 83, about 82, about 81, about 80 percent identity to the sequence. In embodiments, the zinc finger targets one or more sites selected from TABLES 13-17.

In embodiments, the targeting element comprises a nucleic acid binding component of a gene-editing system. In embodiments, the helper enzyme or variant thereof and the targeting element are connected. In embodiments, the helper enzyme and the targeting element are fused to one another or linked via a linker to one another. In embodiments, the composition comprises a linker connecting the helper enzyme and the targeting element. In embodiments, the linker comprises less than about 25 amino acids or 75 nucleotides. In embodiments, the flexible linker is of about 20, or about 30, or about 40, or about 50, or about 60 amino acid residues. In embodiments, the linker comprises about 10 amino acids to about 20 amino acids or about 12 amino acids to about 15 amino acids, or about 30 nucleotides to about 60 nucleotides or about 36 nucleotides to about 45 nucleotides. In embodiments, the linker is a covalent or non-covalent linker.

In embodiments, the linker is a flexible linker. In embodiments, the flexible linker is substantially comprised of glycine and serine residues, optionally wherein the flexible linker comprises (Gly₄Ser)ₙ, where n is an integer from 1-12. In embodiments, the linker is or comprises (GSS)4 or the linker is GS flanked on either side of a DNA binding domain, optionally TALE and ZnF. In embodiments, the linker comprises an amino acid sequence of AKLAGGAPAVGGGPKAADKFAATGGS (SEQ ID NO: 8) or a variant thereof having a substitution or deletion. In embodiments, the linker connects the targeting element to the N-terminus of the helper enzyme or connects the targeting element within the helper enzyme. In embodiment, the linker is a non-covalent linker. In embodiments, the non-covalent linker comprises an epitope tag. In embodiments, the non-covalent linker comprises an ALFA tag. In embodiments, the ALFA tag further comprises recombinant variable domains of heavy-chain-only antibodies (VHH) (NANOBODY). In embodiments, the non-covalent linker comprises recombinant variable domains of heavy-chain-only antibodies (VHH) (NANOBODY). In embodiments, the non-covalent linker comprises recombinant variable domains of heavy-chain-only antibodies (VHH) (NANOBODY) binding to ALFA tagged protein (NbALFA). In embodiments, the linker comprises is a monobody. In embodiments, the linker comprises anis an ALFAlfa tag and monobody. In embodiments, the TnsD comprises a nucleic acid binding component of a gene-editing system. In embodiments, the enzyme or variant thereof (optionally, wherein the enzyme is a helper enzyme, optionally, wherein the helper enzyme is reconstructed from Myotis lucifugus) and the TnsD are connected. In embodiments, the targeting element comprises a TniQ subdomain of TnsD. In embodiments, the helper enzyme and the TniQ subdomain of TnsD are fused to one another or linked via a linker to one another. In embodiments, the linker is a flexible linker. In embodiments, the flexible linker is substantially comprised of glycine and serine residues, optionally wherein the flexible linker comprises (Gly₄Ser)ₙ, where n is an integer from 1-12. In embodiments, the flexible linker is of about 20, or about 30, or about 40, or about 50, or about 60 amino acid residues. In embodiments, the helper enzyme is directly fused to the N-terminus of the TniQ subdomain of TnsD.

In embodiments, the E. coli TnsD comprises at least about 90%, or at least about 95%, or at least about 96%, or at least about 97%, or at least about 98%, or at least about 99% identity to an amino acid sequence of SEQ ID NO: 12. In embodiments, the TnsD comprises a truncated TnsD. In embodiments, the TnsD comprises the TniQ subdomain. In embodiments, the TnsD is truncated at its C-terminus. In embodiments, the TnsD is truncated at its N-terminus. In embodiments, the TnsD or variant thereof comprises a zinc finger motif. In embodiments, the zinc finger motif comprises a C3H-type motif (e.g., CCCH).

```
SEQ ID NO: 12: amino acid sequence of E. coli TnsD (508 amino acids)
    1 MRNFPVPYSN ELIYSTIARA GVYQGIVSPK QLLDEVYGNR KVVATLGLPS HLGVIARHLH
  61 QTGRYAVQQL IYEHTLFPLY APFVGKERRD EAIRLMEYQA QGAVHLMLGV AASRVKSDNR
121 FRYCPDCVAL QLNRYGEAFW QRDWYLPALP YCPKHGALVE FDRAVDDHRH QFWALGHTEL
181 LSDYPKDSLS QLTALAAYIA PLLDAPRAQE LSPSLEQWTL FYQRLAQDLG LTKSKHIRHD
241 LVAERVRQTF SDEALEKLDL KLAENKDTCW LKSIFRKHRK AFSYLQHSIV WQALLPKLTV
301 IEALQQASAL TEHSITTRPV SQSVQPNSED LSVKHKDWQQ LVHKYQGIKA ARQSLEGGVL
361 YAWLYRHDRD WLVHWNQQHQ QERLAPAPRV DWNQRDRIAV RQLLRIIKRL DSSLDHPRAT
421 SSWLLKQTPN GTSLAKNLQK LPLVALCLKR YSESVEDYQI RRISQAFIKL KQEDVELRRW
481 RLLRSATLSK ERITEEAQRF LEMVYGEE
```

In embodiments, the TnsD binds at or near an attTn7 attachment site. In embodiments, the TnsD binds at or near a region downstream of the glmS gene. GlmS (L-glucosamine-fructose-6-phosphate aminotransferase) is highly conserved and found in a wide variety of organisms from bacteria to humans. In embodiments, the TnsD binding region of glmS encodes the active site region of GlmS. In embodiments, TnsD binds at or near the human homologs of glmS, e.g., gfpt-1 and gfpt-2. In embodiments, TnsD binds the human glm S homologs gfpt-1 and gfpt-2. In embodiments, the transgene is inserted into attTn7.

In embodiments, the helper enzyme or variant thereof is able to directly or indirectly cause transposition of a target gene. In embodiments, the helper enzyme or variant thereof is able to directly or indirectly interact and/or form a complex with one or more proteins or nucleic acids.

In embodiments, there is provided a composition comprising a helper enzyme or a nucleic acid encoding the helper enzyme comprising: an amino acid sequence having at least about 80% sequence identity to SEQ ID NO: 1 and a non-polar aliphatic amino acid at position 2 of SEQ ID NO: 1, or a position corresponding thereto, and amino acid substitutions comprising S8X, C13X, R333K, K334A, N335A, R336K, and D416X of SEQ ID NO: 1, or a substitution at a position corresponding thereto.

In embodiments, there is provided a composition comprising (a) a helper enzyme or a nucleic acid encoding the helper enzyme and (b) a targeting element or a nucleic acid encoding the targeting element, wherein: the helper enzyme comprises an amino acid sequence having at least about 80% sequence identity to SEQ ID NO: 1 and has a non-polar aliphatic amino acid at position 2 of SEQ ID NO: 1 and a substitution at a position corresponding thereto, and amino acid substitutions comprising S8X, C13X, R333K, K334A, N335A, R336K, and D416X of SEQ ID NO: 1, or a substitution at a position corresponding thereto.

In embodiments, there is provided a composition comprising a helper enzyme or a nucleic acid encoding the helper enzyme comprising: an amino acid sequence having at least about 80% sequence identity to SEQ ID NO: 1 and a non-polar aliphatic amino acid at position 2 of SEQ ID NO: 1, or a position corresponding thereto, and amino acid substitutions comprising S8X, C13X, R333K, N335A, R336K, and D416X of SEQ ID NO: 1, or a substitution at a position corresponding thereto.

In embodiments, there is provided a composition comprising (a) a helper enzyme or a nucleic acid encoding the helper enzyme and (b) a targeting element or a nucleic acid encoding the targeting element, wherein: an amino acid sequence having at least about 80% sequence identity to SEQ ID NO: 1 and a non-polar aliphatic amino acid at position 2 of SEQ ID NO: 1, or a position corresponding thereto, and amino acid substitutions comprising S8X, C13X, R333K, N335A, R336K, and D416X of SEQ ID NO: 1, or a substitution at a position corresponding thereto.

### Construct

In some embodiments, the composition (e.g., without limitation, a hyperactive helper of the present disclosure), system, or method further comprising a nucleic acid encoding a donor comprising a transgene to be integrated. In some embodiments, the transgene is defective or substantially absent in a disease state. In some embodiments, the transgene comprises a cargo nucleic acid sequence and a first and a second donor end sequences. In some embodiments, the cargo nucleic acid sequence is flanked by the first and the second donor end sequences.

In some embodiments, the donor end sequences are selected from nucleotide sequences of SEQ ID NO: 3 and/or SEQ ID NO: 4, or a nucleotide sequence having at least about 90% identity thereto.

```
SEQ ID NO: 3: hyperactive helper Left ITR (157 bp)
The left ITR retains recognition activity when the
underlined nucleotides are deleted (80 bp).
    1 ttaacacttg gattgcggga aacgagttaa gtcggctcgc gtgaattgcg cgtactccgc
  61 gggagccgtc ttaactcggt tcatatagat ttgcggtgga gtgcgggaaa cgtgtaaact
121 cgggccgatt gtaactgcgt attaccaaat atttgtt
SEQ ID NO: 4: hyperactive helper Right ITR (212 bp)
The right ITR retains recognition activity when the
underlined nucleotides are deleted (80 bp).
    1 aattatttat gtactgaata gataaaaaaa tgtctgtgat tgaataaatt ttcatttttt
  61 acacaagaaa ccgaaaattt catttcaatc gaacccatac ttcaaaagat ataggcattt
121 taaactaact ctgattttgc gcgggaaacc taaataattg cccgcgccat cttatatttt
181 ggcgggaaat tcacccgaca ccgtagtgtt aa
```

In some embodiments, the end sequences include at least one repeat from a nucleotide sequence having at least about 90% identity to the nucleotide sequence of SEQ ID NO: 3. In some embodiments, the at least one repeat from the nucleotide sequence having at least about 90% identity to the nucleotide sequence of SEQ ID NO: 3 is positioned at the 5′ end of the donor. In some embodiments, the end sequences can further include at least one repeat from a nucleotide sequence having at least about 90% identity to the nucleotide sequence of SEQ ID NO: 4. In some embodiments, the at least one repeat from the nucleotide sequence having at least about 90% identity to the nucleotide sequence of SEQ ID NO: 4 is positioned at the 3′ end of the donor.

In embodiments, the left ITR comprises a nucleotide sequence of ttaacacttggattgogggaaacgagttaa gtcggctcgcgtgaattgcgcgtactccgcgggagccgtcttaactc (SEQ ID NO: 878), or a variant thereof comprising substitutions or deletions.

In embodiments, the right ITR comprises a nucleotide sequence of catttcaato gaacccatac ttcaaaagat ataggcattt taaactaact ctgattttgc gogggaaacc taaataattg cccgcgccat cttatatttt ggcgggaaat tcacccgaca ccgtagtgtt aa (SEQ ID NO: 879), or a variant thereof comprising substitutions or deletions.

In embodiments, the ITRs or the end sequences are selected from SEQ ID NO: 878 and SEQ ID NO: 879, or a nucleotide sequence having at least about 90% identity thereto, or at least about 93%, or at least about 95%, or at least about 97%, or at least about 98%, or at least about 99% identity thereto.

In embodiments, the end sequences include at least one repeat with a nucleotide sequence having at least about 90% identity to the nucleotide sequence of SEQ ID NO: 878. In embodiments, the at least one repeat from the nucleotide sequence having at least about 90% identity, or at least about 93%, or at least about 95%, or at least about 97%, or at least about 98%, or at least about 99% identity to the nucleotide sequence of SEQ ID NO: 878 is positioned at the 5′ end of the donor. In embodiments, the end sequences can further include at least one repeat with a nucleotide sequence having at least about 90% identity, or at least about 93%, or at least about 95%, or at least about 97%, or at least about 98%, or at least about 99% identity to the nucleotide sequence of SEQ ID NO: 879. In embodiments, the at least one repeat from the nucleotide sequence having at least about 90%, or at least about 93%, or at least about 95%, or at least about 97%, or at least about 98%, or at least about 99% identity to the nucleotide sequence of SEQ ID NO: 879 is positioned at the 3′ end of the donor.

In some embodiments, the helper enzyme or variant thereof is incorporated into a vector or a vector-like particle. In some embodiments, the vector or a vector-like particle comprises one or more expression cassettes. In some embodiments, the vector or a vector-like particle comprises one expression cassette. In some embodiments, the expression cassette further comprises the helper enzyme or variant thereof, the transgene, the donor end sequences, or a combination thereof.

In some embodiments, the helper enzyme or variant thereof, the transgene, the donor end sequences, or a combination thereof are incorporated into one or more vectors or vector-like particles. In some embodiments, the helper enzyme or variant thereof, the transgene, the donor end sequences, or combination thereof are incorporated into a same vector or vector-like particle. In some embodiments, the helper enzyme or variant thereof, the transgene, the donor end sequences, or combination thereof is incorporated into different vectors vector-like particles. In some embodiments, the vector or vector-like particle is nonviral. In some embodiments, the composition comprises DNA, RNA, or both. In some embodiments, the helper enzyme or variant thereof is in the form of RNA.

In embodiments, the composition is a nucleic acid, optionally an RNA. In embodiments, the RNA is or comprises messenger RNA (mRNA). In embodiments, the mRNA is or comprises modified mRNA (mmRNA). In embodiments, the mmRNA comprises one or more of a 5′-m7G cap (cap0, cap1, or cap2), a pseudouridine or n-methyl-pseudouridine substitution, and a poly-A tail of about 30, or of about 50, or of about 100, or of about 150 nucleotides in length.

In embodiments, the donor is under the control of at least one tissue-specific promoter. In embodiments, the at least one tissue-specific promoter is a single promoter. In embodiments, the at least one tissue-specific promoter is under the control of a dual promoter or a tandem promoter.

In embodiments, the transgene to be integrated comprises at least one gene of interest. In embodiments, the transgene to be integrated comprises one gene of interest. In embodiments, the transgene to be integrated comprises two genes of interest.

In embodiments, the at least one gene of interest comprises peptides for linking genes of interest. In embodiments, the peptides are 2A self-cleaving peptides, or functional variants thereof, wherein the 2A self-cleaving peptide is optionally selected from P2A, E2A, F2A, and T2A, or derivative thereof.

In embodiments, the at least one gene of interest is linked to polynucleotide comprising a sequence comprising a 5′-miRNA, a sense and antisense miRNA pair, and/or a 3′-miRNA.

In embodiments, the donor is used in combination with a gene silencing construct. In embodiments, there is provided a method of gene therapy in a cell comprising contacting the cell with a construct comprising the helper enzyme and/or donor or transgene described herein and/or a gene silencing construct. In embodiments, there is provided a method of gene replacement and silencing comprising contacting the cell with a construct comprising the helper enzyme and/or donor or transgene described herein and/or a gene silencing construct. In embodiments, there is provided a method of gene therapy in a subject comprising administering a construct comprising the helper enzyme and/or donor or transgene described herein and/or a gene silencing construct. In embodiments, there is provided a method of gene replacement and silencing in a subject comprising administering a construct comprising the helper enzyme and/or donor or transgene described herein and/or a gene silencing construct. In embodiments, the donor or transgene described herein and the gene silencing construct are separate constructs. In embodiments, the donor or transgene described herein and the gene silencing construct are separate DNA constructs.

In embodiments, the donor is a dual gene construct. In embodiments, the donor is dual gene construct which comprises DNA. In embodiments, the donor is a bicistronic construct. In embodiments, the donor is a multicistrionic construct. In embodiments, the bicistronic construct allows for the contemporaneous expression of two proteins, e.g., separately from the same RNA transcript. In embodiments, the multicistrionic construct allows for the contemporaneous expression of multiple proteins, e.g., separately from the same RNA transcript.

In embodiments, the bicistronic and/or multicistronic construct comprises a gene of interest and a genetic silencing element. In embodiments, the genetic silencing element provides regulation of gene expression in a cell to prevent, reduce, or ablate the expression of a certain gene. In embodiments, the gene silencing element is capable of silencing during either transcription or translation. In embodiments, the gene silencing element is capable of gene knockdown or knockout. Accordingly, in embodiments, the donor is suitable for contemporaneous “knocking in” and “knocking out” of two or more genes. For example, in embodiments, a gene of interest is provided to a cell to have a beneficial effect and a deleterious gene is knocked out of a cell to reduce or eliminate a deleterious effect.

In embodiments, the gene silencing element is or comprises an RNA-based gene inhibitor or silencer. In embodiments, the gene silencing element is or comprises a short interfering RNA (siRNA), a microRNA (miRNA) and/or a short hairpin RNA (shRNA). embodiments, the donor is a bicistronic and/or multicistronic construct comprising one or more genes of interest, e.g., a transgene to be integrated, optionally wherein the transgene is defective or substantially absent in a disease state and one or more gene silencing element, e.g., one or more siRNA, miRNA, and shRNA. In embodiments, the donor is a bicistronic and/or multicistronic construct comprising one or more genes of interest, e.g., a transgene to be integrated, optionally wherein the transgene is defective or substantially absent in a disease state and one or more gene silencing element, e.g., one or more siRNA, miRNA, and shRNA and the donor is flanked by a first and a second donor end sequences.

In embodiments, the present compositions and methods provide for the helper enzyme or variant thereof excising and/or integrating both one or more one or more genes of interest, e.g., a transgene to be integrated, and one or more gene silencing element, e.g., one or more siRNA, miRNA, and shRNA. In embodiments, the present compositions and methods provide for gene replacement and silencing via a signal donor construct.

### N or C Terminal Deletion Variants

In aspects, the present disclosure further provides a hyperactive helper enzyme with a deletion of various amino acids at either the N or C terminus. In embodiments, the hyperactive helper enzyme comprises a deletion in the N-terminus. In embodiments, the hyperactive helper enzyme comprises a deletion in the C-terminus. In embodiments, the deletion in the N or C termini begins at various positions. In embodiments, the deletion in the N or C termini comprises various lengths.

In embodiments, the helper enzyme of the present disclosure comprises a deletion at positions about 2-35, or about 2-45, or about 2-68, or positions corresponding thereto, wherein the positions are relative to SEQ ID NO: 1. In embodiments, the helper enzyme of the present disclosure comprises a deletion at positions about 2-35, or about 2-45, or about 2-68, or positions corresponding thereto, wherein the positions are relative to SEQ ID NO: 2. In embodiments, the helper enzyme of the present disclosure comprises a deletion at positions about 2-35 (SEQ ID NO: 504), or about 2-45 (SEQ ID NO: 506), or about 2-68 (SEQ ID NO: 508), or positions corresponding thereto, wherein the positions are relative to SEQ ID NO: 1 including the mutations in TABLE 1, TABLE 1A, TABLE 2 and TABLE 2A and DNA binders in TABLE 8-17, and 20. In embodiments, the helper enzyme is an MLT. In embodiments, the deletion comprises an N terminal deletion. In embodiments, the N terminal deletion yields reduced or ablated off-target effects of the helper enzyme compared to the helper enzyme without the N terminal deletion. In embodiments, the helper enzyme comprising the N terminal deletion is or comprises SEQ ID NO: 504. In embodiments, the helper enzyme comprising the N terminal is or comprises SEQ ID NO: 506. In embodiments, the helper enzyme comprising the N terminal is or comprises SEQ ID NO: 508. In embodiments, the mutant with an N or C terminal deletion is further fused to a DNA binder. In embodiments, the DNA binder comprises TALEs, ZnF, and/or dCas.

In embodiments, the hyperactive helper enzyme comprises a deletion from an N- or C-terminus of the polypeptide having an amino acid sequence of SEQ ID NO: 2.

```
SEQ ID NO: 7: Myositis lucifugus (hyperactive helper) nucleotide sequence (NO). 1716 bp
      1 ATGGCCCAGC ACAGCGACTA CCCCGACGAC GAGTTCAGAG CCGATAAGCT GAGTAACTAC
    61 AGCTGCGACA GCGACCTGGA AAACGCCAGC ACATCCGACG AGGACAGCTC TGACGACGAG
  121 GTGATGGTGC GGCCCAGAAC CCTGAGACGG AGAAGAATCA GCAGCTCTAG CAGCGACTCT
  181 GAATCCGACA TCGAGGGCGG CCGGGAAGAG TGGAGCCACG TGGACAACCC TCCTGTTCTG
  241 GAAGATTTTC TGGGCCATCA GGGCCTGAAC ACCGACGCCG TGATCAACAA CATCGAGGAT
  301 GCCGTGAAGC TGTTCATAGG AGATGATTTC TTTGAGTTCC TGGTCGAGGA ATCCAACCGC
  361 TATTACAACC AGAATAGAAA CAACTTCAAG CTGAGCAAGA AAAGCCTGAA GTGGAAGGAC
  421 ATCACCCCTC AGGAGATGAA AAAGTTCCTG GGACTGATCG TTCTGATGGG ACAGGTGCGG
  481 AAGGACAGAA GGGATGATTA CTGGACAACC GAACCTTGGA CCGAGACCCC TTACTTTGGC
  541 AAGACCATGA CCAGAGACAG ATTCAGACAG ATCTGGAAAG CCTGGCACTT CAACAACAAT
  601 GCTGATATCG TGAACGAGTC TGATAGACTG TGTAAAGTGC GGCCAGTGTT GGATTACTTC
  661 GTGCCTAAGT TCATCAACAT CTATAAGCCT CACCAGCAGC TGAGCCTGGA TGAAGGCATC
  721 GTGCCCTGGC GGGGCAGACT GTTCTTCAGA GTGTACAATG CTGGCAAGAT CGTCAAATAC
  781 GGCATCCTGG TGCGCCTTCT GTGCGAGAGC GATACAGGCT ACATCTGTAA TATGGAAATC
  841 TACTGCGGCG AGGGCAAAAG ACTGCTGGAA ACCATCCAGA CCGTCGTTTC CCCTTATACC
  901 GACAGCTGGT ACCACATCTA CATGGACAAC TACTACAATT CTGTGGCCAA CTGCGAGGCC
  961 CTGATGAAGA ACAAGTTTAG AATCTGCGGC ACAATCAGAA AAAACAGAGG CATCCCTAAG
1021 GACTTCCAGA CCATCTCTCT GAAGAAGGGC GAAACCAAGT TCATCAGAAA GAACGACATC
1081 CTGCTCCAAG TGTGGCAGTC CAAGAAACCC GTGTACCTGA TCAGCAGCAT CCATAGCGCC
1141 GAGATGGAAG AAAGCCAGAA CATCGACAGA ACAAGCAAGA AGAAGATCGT GAAGCCCAAT
1201 GCTCTGATCG ACTACAACAA GCACATGAAA GGCGTGGACC GGGCCGACCA GTACCTGTCT
1261 TATTACTCTA TCCTGAGAAG AACAGTGAAA TGGACCAAGA GACTGGCCAT GTACATGATC
1321 AATTGCGCCC TGTTCAACAG CTACGCCGTG TACAAGTCCG TGCGACAAAG AAAAATGGGA
1381 TTCAAGATGT TCCTGAAGCA GACAGCCATC CACTGGCTGA CAGACGACAT TCCTGAGGAC
1441 ATGGACATTG TGCCAGATCT GCAACCTGTG CCCAGCACCT CTGGTATGAG AGCTAAGCCT
1501 CCCACCAGCG ATCCTCCATG TAGACTGAGC ATGGACATGC GGAAGCACAC CCTGCAGGCC
1561 ATCGTCGGCA GCGGCAAGAA GAAGAACATC CTTAGACGGT GCAGGGTGTG CAGCGTGCAC
1621 AAGCTGCGGA GCGAGACTCG GTACATGTGC AAGTTTTGCA ACATTCCCCT GCACAAGGGA
1681 GCCTGCTTCG AGAAGTACCA CACCCTGAAG AATTAC
SEQ ID NO: 2: Myositis lucifugus (hyperactive helper) amino acid sequence (NO). 572 aa
      1 MAQHSDYPDD EFRADKLSNY SCDSDLENAS TSDEDSSDDE VMVRPRTLRR RRISSSSSDS
    61 ESDIEGGREE WSHVDNPPVL EDELGHQGLN TDAVINNIED AVKLFIGDDF FEELVEESNR
  121 YYNQNRNNFK LSKKSLKWKD ITPQEMKKEL GLIVLMGQVR KDRRDDYWTT EPWTETPYFG
  181 KTMTRDRFRQ IWKAWHENNN ADIVNESDRL CKVRPVLDYF VPKFINIYKP HQQLSLDEGI
  241 VPWRGRLFFR VYNAGKIVKY GILVRLLCES DTGYICNMEI YCGEGKRLLE TIQTVVSPYT
  301 DSWYHIYMDN YYNSVANCEA LMKNKFRICG TIRKNRGIPK DFQTISLKKG ETKFIRKNDI
  361 LLQVWQSKKP VYLISSIHSA EMEESQNIDR TSKKKIVKPN ALIDYNKHMK GVDRADQYLS
  421 YYSILRRTVK WTKRLAMYMI NCALENSYAV YKSVRQRKMG FKMELKQTAI HWLTDDIPED
  481 MDIVPDLQPV PSTSGMRAKP PTSDPPCRLS MDMRKHTLQA IVGSGKKKNI LRRCRVCSVH
  541 KLRSETRYMC KFCNIPLHKG ACFEKYHTLK NY
```

In embodiments, the hyperactive helper enzyme comprises a deletion of about 5, or about 10, or about 20, or about 30, or about 40, or about 50, or about 60, or about 70, or about 80, or about 90, or about 100, or about 110, or about 120, or about 130, or about 140, or about 150, or about 160 amino acids from an N-terminus of the polypeptide having an amino acid sequence of SEQ ID NO: 2, or a sequence having at least about 90% identity thereto.

In embodiments, the hyperactive helper enzyme comprises a deletion of about 5, or about 10, or about 20, or about 30, or about 40, or about 50, or about 60, or about 70, or about 80, or about 90, or about 100, or about 110, or about 120, or about 130, or about 140, or about 150, or about 160 amino acids from an N-terminus of the polypeptide having an amino acid sequence of SEQ ID NO: 1, or a sequence having at least about 90% identity thereto.

In embodiments, the hyperactive helper enzyme with deletion from the N-terminus comprises SEQ ID NO: 504, SEQ ID NO: 506, SEQ ID NO: 508, or SEQ ID NO: 510, or a sequence having at least about 90% identity thereto.

```
SEQ ID NO: 503: N-terminal deletion Myositis lucifugus (hyperactive
helper) nucleotide sequence (N1; nucleotide 4-105 deletion).
1614 bp
      1 ATGAGCTCTG ACGACGAGGT GATGGTGCGG CCCAGAACCC TGAGACGGAG AAGAATCAGC
    61 AGCTCTAGCA GCGACTCTGA ATCCGACATC GAGGG CGGCC GGGAAGAGTG GAGCCACGTG
  121 GACAACCCTC CTGTTCTGGA AGATTTTCTG GGCCATCAGG GCCTGAACAC CGACGCCGTG
  181 ATCAACAACA TCGAGGATGC CGTGAAGCTG TTCATAGGAG ATGATTTCTT TGAGTTCCTG
  241 GTCGAGGAAT CCAACCGCTA TTACAACCAG AATAGAAACA ACTTCAAGCT GAGCAAGAAA
  301 AGCCTGAAGT GGAAGGACAT CACCCCTCAG GAGATGAAAA AGTTCCTGGG ACTGATCGTT
  361 CTGATGGGAC AGGTGCGGAA GGACAGAAGG GATGATTACT GGACAACCGA ACCTTGGACC
  421 GAGACCCCTT ACTTTGGCAA GACCATGACC AGAGACAGAT TCAGACAGAT CTGGAAAGCC
  481 TGGCACTTCA ACAACAATGC TGATATCGTG AACGAGTCTG ATAGACTGTG TAAAGTGCGG
  541 CCAGTGTTGG ATTACTTCGT GCCTAAGTTC ATCAACATCT ATAAGCCTCA CCAGCAGCTG
  601 AGCCTGGATG AAGGCATCGT GCCCTGGCGG GGCAGACTGT TCTTCAGAGT GTACAATGCT
  661 GGCAAGATCG TCAAATACGG CATCCTGGTG CGCCTTCTGT GCGAGAGCGA TACAGGCTAC
  721 ATCTGTAATA TGGAAATCTA CTGCGGCGAG GGCAAAAGAC TGCTGGAAAC CATCCAGACC
  781 GTCGTTTCCC CTTATACCGA CAGCTGGTAC CACATCTACA TGGACAACTA CTACAATTCT
  841 GTGGCCAACT GCGAGGCCCT GATGAAGAAC AAGTTTAGAA TCTGCGGCAC AATCAGAAAA
  901 AACAGAGGCA TCCCTAAGGA CTTCCAGACC ATCTCTCTGA AGAAGGGCGA AACCAAGTTC
  961 ATCAGAAAGA ACGACATCCT GCTCCAAGTG TGGCAGTCCA AGAAACCCGT GTACCTGATC
1021 AGCAGCATCC ATAGCGCCGA GATGGAAGAA AGCCAGAACA TCGACAGAAC AAGCAAGAAG
1081 AAGATCGTGA AGCCCAATGC TCTGATCGAC TACAACAAGC ACATGAAAGG CGTGGACCGG
1141 GCCGACCAGT ACCTGTCTTA TTACTCTATC CTGAGAAGAA CAGTGAAATG GACCAAGAGA
1201 CTGGCCATGT ACATGATCAA TTGCGCCCTG TTCAACAGCT ACGCCGTGTA CAAGTCCGTG
1261 CGACAAAGAA AAATGGGATT CAAGATGTTC CTGAAGCAGA CAGCCATCCA CTGGCTGACA
1321 GACGACATTC CTGAGGACAT GGACATTGTG CCAGATCTGC AACCTGTGCC CAGCACCTCT
1381 GGTATGAGAG CTAAGCCTCC CACCAGCGAT CCTCCATGTA GACTGAGCAT GGACATGCGG
1441 AAGCACACCC TGCAGGCCAT CGTCGGCAGC GGCAAGAAGA AGAACATCCT TAGACGGTGC
1501 AGGGTGTGCA GCGTGCACAA GCTGCGGAGC GAGACTCGGT ACATGTGCAA GTTTTGCAAC
1561 ATTCCCCTGC ACAAGGGAGC CTGCTTCGAG AAGTACCACA CCCTGAAGAA TTAC
SEQ ID NO: 504: Myositis lucifugus (hyperactive helper) amino
acid sequence (N1, amino acid 2-35 deletion). 538 aa
      1 MSSDDEVMVR PRTLRRRRIS SSSSDSESDI EGGREEWSHV DNPPVLEDFL GHQGLNTDAV
    61 INNIEDAVKL FIGDDFFEFL VEESNRYYNQ NRNNFKLSKK SLKWKDITPQ EMKKELGLIV
  121 LMGQVRKDRR DDYWTTEPWT ETPYFGKTMT RDRFRQIWKA WHENNNADIV NESDRLCKVR
  181 PVLDYFVPKF INIYKPHQQL SLDEGIVPWR GRLFFRVYNA GKIVKYGILV RLLCESDTGY
  241 ICNMEIYCGE GKRLLETIQT VVSPYTDSWY HIYMDNYYNS VANCEALMKN KFRICGTIRK
  301 NRGIPKDFQT ISLKKGETKE IRKNDILLQV WQSKKPVYLI SSIHSAEMEE SQNIDRTSKK
  361 KIVKPNALID YNKHMKGVDR ADQYLSYYSI LRRTVKWTKR LAMYMINCAL FNSYAVYKSV
  421 RQRKMGFKMF LKQTAIHWLT DDIPEDMDIV PDLQPVPSTS GMRAKPPTSD PPCRLSMDMR
  481 KHTLQAIVGS GKKKNILRRC RVCSVHKLRS ETRYMCKFCN IPLHKGACFE KYHTLKNY
SEQ ID NO: 505: N-terminal deletion Myositis lucifugus (hyperactive helper)
nucleotide sequence (N2; nucleotide 4-135 deletion). 1584 bp
      1 ATGAGAACCC TGAGACGGAG AAGAATCAGC AGCTCTAGCA GCGACTCTGA ATCCGACATC
    61 GAGGGCGGCC GGGAAGAGTG GAGCCACGTG GACAACCCTC CTGTTCTGGA AGATTTTCTG
  121 GGCCATCAGG GCCTGAACAC CGACGCCGTG ATCAACAACA TCGAGGATGC CGTGAAGCTG
  181 TTCATAGGAG ATGATTTCTT TGAGTTCCTG GTCGAGGAAT CCAACCGCTA TTACAACCAG
  241 AATAGAAACA ACTTCAAGCT GAGCAAGAAA AGCCTGAAGT GGAAGGACAT CACCCCTCAG
  301 GAGATGAAAA AGTTCCTGGG ACTGATCGTT CTGATGGGAC AGGTGCGGAA GGACAGAAGG
  361 GATGATTACT GGACAACCGA ACCTTGGACC GAGACCCCTT ACTTTGGCAA GACCATGACC
  421 AGAGACAGAT TCAGACAGAT CTGGAAAGCC TGGCACTTCA ACAACAATGC TGATATCGTG
  481 AACGAGTCTG ATAGACTGTG TAAAGTGCGG CCAGTGTTGG ATTACTTCGT GCCTAAGTTC
  541 ATCAACATCT ATAAGCCTCA CCAGCAGCTG AGCCTGGATG AAGGCATCGT GCCCTGGCGG
  601 GGCAGACTGT TCTTCAGAGT GTACAATGCT GGCAAGATCG TCAAATACGG CATCCTGGTG
  661 CGCCTTCTGT GCGAGAGCGA TACAGGCTAC ATCTGTAATA TGGAAATCTA CTGCGGCGAG
  721 GGCAAAAGAC TGCTGGAAAC CATCCAGACC GTCGTTTCCC CTTATACCGA CAGCTGGTAC
  781 CACATCTACA TGGACAACTA CTACAATTCT GTGGCCAACT GCGAGGCCCT GATGAAGAAC
  841 AAGTTTAGAA TCTGCGGCAC AATCAGAAAA AACAGAGGCA TCCCTAAGGA CTTCCAGACC
  901 ATCTCTCTGA AGAAGGGCGA AACCAAGTTC ATCAGAAAGA ACGACATCCT GCTCCAAGTG
  961 TGGCAGTCCA AGAAACCCGT GTACCTGATC AGCAGCATCC ATAGCGCCGA GATGGAAGAA
1021 AGCCAGAACA TCGACAGAAC AAGCAAGAAG AAGATCGTGA AGCCCAATGC TCTGATCGAC
1081 TACAACAAGC ACATGAAAGG CGTGGACCGG GCCGACCAGT ACCTGTCTTA TTACTCTATC
1141 CTGAGAAGAA CAGTGAAATG GACCAAGAGA CTGGCCATGT ACATGATCAA TTGCGCCCTG
1201 TTCAACAGCT ACGCCGTGTA CAAGTCCGTG CGACAAAGAA AAATGGGATT CAAGATGTTC
1261 CTGAAGCAGA CAGCCATCCA CTGGCTGACA GACGACATTC CTGAGGACAT GGACATTGTG
1321 CCAGATCTGC AACCTGTGCC CAGCACCTCT GGTATGAGAG CTAAGCCTCC CACCAGCGAT
1381 CCTCCATGTA GACTGAGCAT GGACATGCGG AAGCACACCC TGCAGGCCAT CGTCGGCAGC
1441 GGCAAGAAGA AGAACATCCT TAGACGGTGC AGGGTGTGCA GCGTGCACAA GCTGCGGAGC
1501 GAGACTCGGT ACATGTGCAA GTTTTGCAAC ATTCCCCTGC ACAAGGGAGC CTGCTTCGAG
1561 AAGTACCACA CCCTGAAGAA TTAC
SEQ ID NO: 506: Myositis lucifugus (hyperactive helper) amino
acid sequence (N2, amino acid 2-45 deletion). 528 aa
      1 MRTLRRRRIS SSSSDSESDI EGGREEWSHV DNPPVLEDEL GHQGLNTDAV INNIEDAVKL
    61 FIGDDFFEFL VEESNRYYNQ NRNNFKLSKK SLKWKDITPQ EMKKELGLIV LMGQVRKDRR
  121 DDYWTTEPWT ETPYFGKTMT RDRFRQIWKA WHENNNADIV NESDRLCKVR PVLDYFVPKF
  181 INIYKPHQQL SLDEGIVPWR GRLFFRVYNA GKIVKYGILV RLLCESDTGY ICNMEIYCGE
  241 GKRLLETIQT VVSPYTDSWY HIYMDNYYNS VANCEALMKN KFRICGTIRK NRGIPKDEQT
  301 ISLKKGETKF IRKNDILLQV WQSKKPVYLI SSIHSAEMEE SQNIDRTSKK KIVKPNALID
  361 YNKHMKGVDR ADQYLSYYSI LRRTVKWTKR LAMYMINCAL FNSYAVYKSV RQRKMGFKMF
  421 LKQTAIHWLT DDIPEDMDIV PDLQPVPSTS GMRAKPPTSD PPCRLSMDMR KHTLQAIVGS
  481 GKKKNILRRC RVCSVHKLRS ETRYMCKFCN IPLHKGACFE KYHTLKNY
SEQ ID NO: 507: N-terminal deletion Myositis lucifugus (hyperactive
helper) nucleotide sequence (N3; nucleotide 4-204 deletion). 1515 bp
      1 ATGGAAGAGT GGAGCCACGT GGACAACCCT CCTGTTCTGG AAGATTTTCT GGGCCATCAG
    61 GGCCTGAACA CCGACGCCGT GATCAACAAC ATCGAGGATG CCGTGAAGCT GTTCATAGGA
  121 GATGATTTCT TTGAGTTCCT GGTCGAGGAA TCCAACCGCT ATTACAACCA GAATAGAAAC
  181 AACTTCAAGC TGAGCAAGAA AAGCCTGAAG TGGAAGGACA TCACCCCTCA GGAGATGAAA
  241 AAGTTCCTGG GACTGATCGT TCTGATGGGA CAGGTGCGGA AGGACAGAAG GGATGATTAC
  301 TGGACAACCG AACCTTGGAC CGAGACCCCT TACTTTGGCA AGACCATGAC CAGAGACAGA
  361 TTCAGACAGA TCTGGAAAGC CTGGCACTTC AACAACAATG CTGATATCGT GAACGAGTCT
  421 GATAGACTGT GTAAAGTGCG GCCAGTGTTG GATTACTTCG TGCCTAAGTT CATCAACATC
  481 TATAAGCCTC ACCAGCAGCT GAGCCTGGAT GAAGGCATCG TGCCCTGGCG GGGCAGACTG
  541 TTCTTCAGAG TGTACAATGC TGGCAAGATC GTCAAATACG GCATCCTGGT GCGCCTTCTG
  601 TGCGAGAGCG ATACAGGCTA CATCTGTAAT ATGGAAATCT ACTGCGGCGA GGGCAAAAGA
  661 CTGCTGGAAA CCATCCAGAC CGTCGTTTCC CCTTATACCG ACAGCTGGTA CCACATCTAC
  721 ATGGACAACT ACTACAATTC TGTGGCCAAC TGCGAGGCCC TGATGAAGAA CAAGTTTAGA
  781 ATCTGCGGCA CAATCAGAAA AAACAGAGGC ATCCCTAAGG ACTTCCAGAC CATCTCTCTG
  841 AAGAAGGGCG AAACCAAGTT CATCAGAAAG AACGACATCC TGCTCCAAGT GTGGCAGTCC
  901 AAGAAACCCG TGTACCTGAT CAGCAGCATC CATAGCGCCG AGATGGAAGA AAGCCAGAAC
  961 ATCGACAGAA CAAGCAAGAA GAAGATCGTG AAGCCCAATG CTCTGATCGA CTACAACAAG
1021 CACATGAAAG GCGTGGACCG GGCCGACCAG TACCTGTCTT ATTACTCTAT CCTGAGAAGA
1081 ACAGTGAAAT GGACCAAGAG ACTGGCCATG TACATGATCA ATTGCGCCCT GTTCAACAGC
1141 TACGCCGTGT ACAAGTCCGT GCGACAAAGA AAAATGGGAT TCAAGATGTT CCTGAAGCAG
1201 ACAGCCATCC ACTGGCTGAC AGACGACATT CCTGAGGACA TGGACATTGT GCCAGATCTG
1261 CAACCTGTGC CCAGCACCTC TGGTATGAGA GCTAAGCCTC CCACCAGCGA TCCTCCATGT
1321 AGACTGAGCA TGGACATGCG GAAGCACACC CTGCAGGCCA TCGTCGGCAG CGGCAAGAAG
1381 AAGAACATCC TTAGACGGTG CAGGGTGTGC AGCGTGCACA AGCTGCGGAG CGAGACTCGG
1441 TACATGTGCA AGTTTTGCAA CATTCCCCTG CACAAGGGAG CCTGCTTCGA GAAGTACCAC
1501 ACCCTGAAGA ATTAC
SEQ ID NO: 508: Myositis lucifugus (hyperactive helper)
amino acid sequence (N3, amino acid 2-68 deletion) 505 aa
      1 MEEWSHVDNP PVLEDFLGHQ GLNTDAVINN IEDAVKLFIG DDFFEFLVEE SNRYYNQNRN
    61 NFKLSKKSLK WKDITPQEMK KELGLIVLMG QVRKDRRDDY WTTEPWTETP YFGKTMTRDR
  121 FRQIWKAWHF NNNADIVNES DRLCKVRPVL DYFVPKFINI YKPHQQLSLD EGIVPWRGRL
  181 FFRVYNAGKI VKYGILVRLL CESDTGYICN MEIYCGEGKR LLETIQTVVS PYTDSWYHIY
  241 MDNYYNSVAN CEALMKNKER ICGTIRKNRG IPKDFQTISL KKGETKFIRK NDILLQVWQS
  301 KKPVYLISSI HSAEMEESQN IDRTSKKKIV KPNALIDYNK HMKGVDRADQ YLSYYSILRR
  361 TVKWTKRLAM YMINCALENS YAVYKSVRQR KMGFKMELKQ TAIHWLTDDI PEDMDIVPDL
  421 QPVPSTSGMR AKPPTSDPPC RLSMDMRKHT LQAIVGSGKK KNILRRCRVC SVHKLRSETR
  481 YMCKFCNIPL HKGACFEKYH TLKNY
SEQ ID NO: 509: N-terminal deletion Myositis lucifugus
(hyperactive helper) nucleotide sequence (N4; nucleotide
4-267 deletion). 1452 bp
      1 ATGAACACCG ACGCCGTGAT CAACAACATC GAGGATGCCG TGAAGCTGTT CATAGGAGAT
    61 GATTTCTTTG AGTTCCTGGT CGAGGAATCC AACCGCTATT ACAACCAGAA TAGAAACAAC
  121 TTCAAGCTGA GCAAGAAAAG CCTGAAGTGG AAGGACATCA CCCCTCAGGA GATGAAAAAG
  181 TTCCTGGGAC TGATCGTTCT GATGGGACAG GTGCGGAAGG ACAGAAGGGA TGATTACTGG
  241 ACAACCGAAC CTTGGACCGA GACCCCTTAC TTTGGCAAGA CCATGACCAG AGACAGATTC
  301 AGACAGATCT GGAAAGCCTG GCACTTCAAC AACAATGCTG ATATCGTGAA CGAGTCTGAT
  361 AGACTGTGTA AAGTGCGGCC AGTGTTGGAT TACTTCGTGC CTAAGTTCAT CAACATCTAT
  421 AAGCCTCACC AGCAGCTGAG CCTGGATGAA GGCATCGTGC CCTGGCGGGG CAGACTGTTC
  481 TTCAGAGTGT ACAATGCTGG CAAGATCGTC AAATACGGCA TCCTGGTGCG CCTTCTGTGC
  541 GAGAGCGATA CAGGCTACAT CTGTAATATG GAAATCTACT GCGGCGAGGG CAAAAGACTG
  601 CTGGAAACCA TCCAGACCGT CGTTTCCCCT TATACCGACA GCTGGTACCA CATCTACATG
  661 GACAACTACT ACAATTCTGT GGCCAACTGC GAGGCCCTGA TGAAGAACAA GTTTAGAATC
  721 TGCGGCACAA TCAGAAAAAA CAGAGGCATC CCTAAGGACT TCCAGACCAT CTCTCTGAAG
  781 AAGGGCGAAA CCAAGTTCAT CAGAAAGAAC GACATCCTGC TCCAAGTGTG GCAGTCCAAG
  841 AAACCCGTGT ACCTGATCAG CAGCATCCAT AGCGCCGAGA TGGAAGAAAG CCAGAACATC
  901 GACAGAACAA GCAAGAAGAA GATCGTGAAG CCCAATGCTC TGATCGACTA CAACAAGCAC
  961 ATGAAAGGCG TGGACCGGGC CGACCAGTAC CTGTCTTATT ACTCTATCCT GAGAAGAACA
1021 GTGAAATGGA CCAAGAGACT GGCCATGTAC ATGATCAATT GCGCCCTGTT CAACAGCTAC
1081 GCCGTGTACA AGTCCGTGCG ACAAAGAAAA ATGGGATTCA AGATGTTCCT GAAGCAGACA
1141 GCCATCCACT GGCTGACAGA CGACATTCCT GAGGACATGG ACATTGTGCC AGATCTGCAA
1201 CCTGTGCCCA GCACCTCTGG TATGAGAGCT AAGCCTCCCA CCAGCGATCC TCCATGTAGA
1261 CTGAGCATGG ACATGCGGAA GCACACCCTG CAGGCCATCG TCGGCAGCGG CAAGAAGAAG
1321 AACATCCTTA GACGGTGCAG GGTGTGCAGC GTGCACAAGC TGCGGAGCGA GACTCGGTAC
1381 ATGTGCAAGT TTTGCAACAT TCCCCTGCAC AAGGGAGCCT GCTTCGAGAA GTACCACACC
1441 CTGAAGAATT AC
SEQ ID NO: 510: Myositis lucifugus (hyperactive helper)
amino acid sequence (N4, amino acid 2-89 deletion).
484 aa
      1 MNTDAVINNI EDAVKLFIGD DEFEFLVEES NRYYNQNRNN FKLSKKSLKW KDITPQEMKK
    61 FLGLIVLMGQ VRKDRRDDYW TTEPWTETPY FGKTMTRDRF RQIWKAWHEN NNADIVNESD
  121 RLCKVRPVLD YFVPKFINIY KPHQQLSLDE GIVPWRGRLF FRVYNAGKIV KYGILVRLLC
  181 ESDTGYICNM EIYCGEGKRL LETIQTVVSP YTDSWYHIYM DNYYNSVANC EALMKNKFRI
  241 CGTIRKNRGI PKDFQTISLK KGETKFIRKN DILLQVWQSK KPVYLISSIH SAEMEESQNI
  301 DRTSKKKIVK PNALIDYNKH MKGVDRADQY LSYYSILRRT VKWTKRLAMY MINCALENSY
  361 AVYKSVRQRK MGFKMELKQT AIHWLTDDIP EDMDIVPDLQ PVPSTSGMRA KPPTSDPPCR
  421 LSMDMRKHTL QAIVGSGKKK NILRRCRVCS VHKLRSETRY MCKFCNIPLH KGACFEKYHT
  481 LKNY
```

In embodiments, the hyperactive helper enzyme comprises a deletion of about 5, or about 10, or about 20, or about 30, or about 40, or about 50, or about 60, or about 70, or about 80, or about 90, or about 100, or about 110, or about 120, or about 130, or about 140, or about 150, or about 160 amino acids from an C-terminus of the polypeptide having an amino acid sequence of SEQ ID NO: 2.

In embodiments, the hyperactive helper enzyme comprises a deletion of about 5, or about 10, or about 20, or about 30, or about 40, or about 50, or about 60, or about 70, or about 80, or about 90, or about 100, or about 110, or about 120, or about 130, or about 140, or about 150, or about 160 amino acids from an C-terminus of the polypeptide having an amino acid sequence of SEQ ID NO: 1.

In embodiments, the hyperactive helper enzyme with deletion from the C-terminus comprises SEQ ID NO: 512 or SEQ ID NO: 514.

```
SEQ ID NO: 511: C-terminal deletion Myositis lucifugus (hyperactive helper)
nucleotide sequence (C1; nucleotide 1663-17 16 deletion). 1662 bp
      1 ATGGCCCAGC ACAGCGACTA CCCCGACGAC GAGTTCAGAG CCGATAAGCT GAGTAACTAC
    61 AGCTGCGACA GCGACCTGGA AAACGCCAGC ACATCCGACG AGGACAGCTC TGACGACGAG
  121 GTGATGGTGC GGCCCAGAAC CCTGAGACGG AGAAGAATCA GCAGCTCTAG CAGCGACTCT
  181 GAATCCGACA TCGAGGGCGG CCGGGAAGAG TGGAGCCACG TGGACAACCC TCCTGTTCTG
  241 GAAGATTTTC TGGGCCATCA GGGCCTGAAC ACCGACGCCG TGATCAACAA CATCGAGGAT
  301 GCCGTGAAGC TGTTCATAGG AGATGATTTC TTTGAGTTCC TGGTCGAGGA ATCCAACCGC
  361 TATTACAACC AGAATAGAAA CAACTTCAAG CTGAGCAAGA AAAGCCTGAA GTGGAAGGAC
  421 ATCACCCCTC AGGAGATGAA AAAGTTCCTG GGACTGATCG TTCTGATGGG ACAGGTGCGG
  481 AAGGACAGAA GGGATGATTA CTGGACAACC GAACCTTGGA CCGAGACCCC TTACTTTGGC
  541 AAGACCATGA CCAGAGACAG ATTCAGACAG ATCTGGAAAG CCTGGCACTT CAACAACAAT
  601 GCTGATATCG TGAACGAGTC TGATAGACTG TGTAAAGTGC GGCCAGTGTT GGATTACTTC
  661 GTGCCTAAGT TCATCAACAT CTATAAGCCT CACCAGCAGC TGAGCCTGGA TGAAGGCATC
  721 GTGCCCTGGC GGGGCAGACT GTTCTTCAGA GTGTACAATG CTGGCAAGAT CGTCAAATAC
  781 GGCATCCTGG TGCGCCTTCT GTGCGAGAGC GATACAGGCT ACATCTGTAA TATGGAAATC
  841 TACTGCGGCG AGGGCAAAAG ACTGCTGGAA ACCATCCAGA CCGTCGTTTC CCCTTATACC
  901 GACAGCTGGT ACCACATCTA CATGGACAAC TACTACAATT CTGTGGCCAA CTGCGAGGCC
  961 CTGATGAAGA ACAAGTTTAG AATCTGCGGC ACAATCAGAA AAAACAGAGG CATCCCTAAG
1021 GACTTCCAGA CCATCTCTCT GAAGAAGGGC GAAACCAAGT TCATCAGAAA GAACGACATC
1081 CTGCTCCAAG TGTGGCAGTC CAAGAAACCC GTGTACCTGA TCAGCAGCAT CCATAGCGCC
1141 GAGATGGAAG AAAGCCAGAA CATCGACAGA ACAAGCAAGA AGAAGATCGT GAAGCCCAAT
1201 GCTCTGATCG ACTACAACAA GCACATGAAA GGCGTGGACC GGGCCGACCA GTACCTGTCT
1261 TATTACTCTA TCCTGAGAAG AACAGTGAAA TGGACCAAGA GACTGGCCAT GTACATGATC
1321 AATTGCGCCC TGTTCAACAG CTACGCCGTG TACAAGTCCG TGCGACAAAG AAAAATGGGA
1381 TTCAAGATGT TCCTGAAGCA GACAGCCATC CACTGGCTGA CAGACGACAT TCCTGAGGAC
1441 ATGGACATTG TGCCAGATCT GCAACCTGTG CCCAGCACCT CTGGTATGAG AGCTAAGCCT
1501 CCCACCAGCG ATCCTCCATG TAGACTGAGC ATGGACATGC GGAAGCACAC CCTGCAGGCC
1561 ATCGTCGGCA GCGGCAAGAA GAAGAACATC CTTAGACGGT GCAGGGTGTG CAGCGTGCAC
1621 AAGCTGCGGA GCGAGACTCG GTACATGTGC AAGTTTTGCA AC
SEQ ID NO: 512: Myositis lucifugus (hyperactive helper) amino acid sequence
(C1, amino acid 555-572 deletion). 554 aa
      1 MAQHSDYPDD EFRADKLSNY SCDSDLENAS TSDEDSSDDE VMVRPRTLRR RRISSSSSDS
    61 ESDIEGGREE WSHVDNPPVL EDFLGHQGLN TDAVINNIED AVKLFIGDDF FEFLVEESNR
  121 YYNQNRNNFK LSKKSLKWKD ITPQEMKKEL GLIVLMGQVR KDRRDDYWTT EPWTETPYFG
  181 KTMTRDRERQ IWKAWHENNN ADIVNESDRL CKVRPVLDYF VPKFINIYKP HQQLSLDEGI
  241 VPWRGRLFFR VYNAGKIVKY GILVRLLCES DTGYICNMEI YCGEGKRLLE TIQTVVSPYT
  301 DSWYHIYMDN YYNSVANCEA LMKNKFRICG TIRKNRGIPK DFQTISLKKG ETKFIRKNDI
  361 LLQVWQSKKP VYLISSIHSA EMEESQNIDR TSKKKIVKPN ALIDYNKHMK GVDRADQYLS
  421 YYSILRRTVK WTKRLAMYMI NCALENSYAV YKSVRORKMG FKMELKQTAI HWLTDDIPED
  481 MDIVPDLQPV PSTSGMRAKP PTSDPPCRLS MDMRKHTLQA IVGSGKKKNI LRRCRVCSVH
  541 KLRSETRYMC KFCN
SEQ ID NO: 513: C-terminal deletion Myositis lucifugus (hyperactive helper)
nucleotide sequence (C2; nucleotide 1588-17 16 deletion). 1587 bp
      1 ATGGCCCAGC ACAGCGACTA CCCCGACGAC GAGTTCAGAG CCGATAAGCT GAGTAACTAC
    61 AGCTGCGACA GCGACCTGGA AAACGCCAGC ACATCCGACG AGGACAGCTC TGACGACGAG
  121 GTGATGGTGC GGCCCAGAAC CCTGAGACGG AGAAGAATCA GCAGCTCTAG CAGCGACTCT
  181 GAATCCGACA TCGAGGGCGG CCGGGAAGAG TGGAGCCACG TGGACAACCC TCCTGTTCTG
  241 GAAGATTTTC TGGGCCATCA GGGCCTGAAC ACCGACGCCG TGATCAACAA CATCGAGGAT
  301 GCCGTGAAGC TGTTCATAGG AGATGATTTC TTTGAGTTCC TGGTCGAGGA ATCCAACCGC
  361 TATTACAACC AGAATAGAAA CAACTTCAAG CTGAGCAAGA AAAGCCTGAA GTGGAAGGAC
  421 ATCACCCCTC AGGAGATGAA AAAGTTCCTG GGACTGATCG TTCTGATGGG ACAGGTGCGG
  481 AAGGACAGAA GGGATGATTA CTGGACAACC GAACCTTGGA CCGAGACCCC TTACTTTGGC
  541 AAGACCATGA CCAGAGACAG ATTCAGACAG ATCTGGAAAG CCTGGCACTT CAACAACAAT
  601 GCTGATATCG TGAACGAGTC TGATAGACTG TGTAAAGTGC GGCCAGTGTT GGATTACTTC
  661 GTGCCTAAGT TCATCAACAT CTATAAGCCT CACCAGCAGC TGAGCCTGGA TGAAGGCATC
  721 GTGCCCTGGC GGGGCAGACT GTTCTTCAGA GTGTACAATG CTGGCAAGAT CGTCAAATAC
  781 GGCATCCTGG TGCGCCTTCT GTGCGAGAGC GATACAGGCT ACATCTGTAA TATGGAAATC
  841 TACTGCGGCG AGGGCAAAAG ACTGCTGGAA ACCATCCAGA CCGTCGTTTC CCCTTATACC
  901 GACAGCTGGT ACCACATCTA CATGGACAAC TACTACAATT CTGTGGCCAA CTGCGAGGCC
  961 CTGATGAAGA ACAAGTTTAG AATCTGCGGC ACAATCAGAA AAAACAGAGG CATCCCTAAG
1021 GACTTCCAGA CCATCTCTCT GAAGAAGGGC GAAACCAAGT TCATCAGAAA GAACGACATC
1081 CTGCTCCAAG TGTGGCAGTC CAAGAAACCC GTGTACCTGA TCAGCAGCAT CCATAGCGCC
1141 GAGATGGAAG AAAGCCAGAA CATCGACAGA ACAAGCAAGA AGAAGATCGT GAAGCCCAAT
1201 GCTCTGATCG ACTACAACAA GCACATGAAA GGCGTGGACC GGGCCGACCA GTACCTGTCT
1261 TATTACTCTA TCCTGAGAAG AACAGTGAAA TGGACCAAGA GACTGGCCAT GTACATGATC
1321 AATTGCGCCC TGTTCAACAG CTACGCCGTG TACAAGTCCG TGCGACAAAG AAAAATGGGA
1381 TTCAAGATGT TCCTGAAGCA GACAGCCATC CACTGGCTGA CAGACGACAT TCCTGAGGAC
1441 ATGGACATTG TGCCAGATCT GCAACCTGTG CCCAGCACCT CTGGTATGAG AGCTAAGCCT
1501 CCCACCAGCG ATCCTCCATG TAGACTGAGC ATGGACATGC GGAAGCACAC CCTGCAGGCC
1561 ATCGTCGGCA GCGGCAAGAA GAAGAAC
SEQ ID NO: 514: Myositis lucifugus (hyperactive helper)
amino acid sequence (C2, amino acid 530-572 deletion).
529 aa
      1 MAQHSDYPDD EFRADKLSNY SCDSDLENAS TSDEDSSDDE VMVRPRTLRR RRISSSSSDS
    61 ESDIEGGREE WSHVDNPPVL EDFLGHQGLN TDAVINNIED AVKLFIGDDF FEELVEESNR
  121 YYNQNRNNFK LSKKSLKWKD ITPQEMKKEL GLIVLMGQVR KDRRDDYWTT EPWTETPYFG
  181 KTMTRDRERQ IWKAWHENNN ADIVNESDRL CKVRPVLDYF VPKFINIYKP HQQLSLDEGI
  241 VPWRGRLFER VYNAGKIVKY GILVRLLCES DTGYICNMEI YCGEGKRLLE TIQTVVSPYT
  301 DSWYHIYMDN YYNSVANCEA LMKNKFRICG TIRKNRGIPK DFQTISLKKG ETKFIRKNDI
  361 LLQVWQSKKP VYLISSIHSA EMEESQNIDR TSKKKIVKPN ALIDYNKHMK GVDRADQYLS
  421 YYSILRRTVK WTKRLAMYMI NCALENSYAV YKSVRORKMG FKMELKQTAI HWLTDDIPED
  481 MDIVPDLQPV PSTSGMRAKP PTSDPPCRLS MDMRKHTLQA IVGSGKKKN
```

In embodiments, the hyperactive helper enzyme comprises a deletion at positions about 1-5, or about 1-15, or about 1-25, or about 1-35, or about 1-45, or about 1-55, or about 1-65, or about 1-75, or about 1-85, or about 1-95, or about 1-105, or about 1-115, or about 1-125, or about 1-135, or about 1-145, or about 1-155 or about 2-35, or about 2-45, or about 2-68, or positions corresponding thereto, wherein the positions are relative to SEQ ID NO: 2.

In embodiments, the hyperactive helper enzyme comprises a deletion at positions about 1-5, or about 1-15, or about 1-25, or about 1-35, or about 1-45, or about 1-55, or about 1-65, or about 1-75, or about 1-85, or about 1-95, or about 1-105, or about 1-115, or about 1-125, or about 1-135, or about 1-145, or about 1-155 or about 2-35, or about 2-45, or about 2-68, or positions corresponding thereto, wherein the positions are relative to SEQ ID NO: 1.

In aspects, the N terminal deletion variant is further fused one or more DNA binders. In embodiments, the DNA binder comprises, without limitation, dCasx, dCas9, dCas12j, TALEs, and ZnF. In embodiments, the DNA binder guides donor insertion to specific genomic sites. In embodiments, the C terminal deletion variant is further fused one or more DNA binders. In embodiments, the N terminal deletion variant is further fused one or more DNA binders at the N-terminus. In embodiments, the N terminal deletion variant is further fused one or more DNA binders at the C-terminus. In embodiments, the C terminal deletion variant is further fused one or more DNA binders at the N-terminus. In embodiments, the C terminal deletion variant is further fused one or more DNA binders at the C-terminus.

In embodiments, the hyperactive helper mutant exhibits improved excision frequencies compared to those without the terminal deletions and/or DNA binders. In embodiments, the hyperactive helper mutant exhibits improved integration frequencies compared to those without the terminal deletions and/or DNA binders. In embodiments, the hyperactive helper mutant exhibits improved excision and integration frequencies compared to those without the terminal deletions and/or DNA binders.

In embodiments, the N or C terminal mutant exhibit different Exc+/Int− frequencies. In embodiments, deletion of either N or C termini can result in MLT mutants with higher excision activity. In embodiments, N-terminal deletion yields a mutant with decreased integration compared to mutant without N-terminal deletion. In embodiments, C-terminal deletion yields a mutant with reduced excision and no integration.

In embodiments, the N or C terminal deletion yields reduced or ablated off-target effects of the helper enzyme compared to the helper enzyme without the N or C terminal deletion.

### Host Cell

In some aspects, the present disclosure further provides a host cell comprising the composition in accordance with embodiments of the present disclosure.

### Methods

In certain embodiments, the present disclosure provides a method for inserting a gene into the genome of a cell, comprising contacting a cell with the composition of the present disclosure or host cell of the present disclosure. In some embodiments, the method further comprises contacting the cell with a polynucleotide encoding a donor. In embodiments, the helper enzyme is suitable of inserting a donor nucleic acid comprising a transgene in a genomic safe harbor site (GSHS) and/or wherein the targeting element is suitable for directing the helper enzyme to a GSHS. In embodiments, a donor DNA and a helper RNA are suitable for transfection at a donor DNA to helper RNA ratio of about 1 to about 4, or about 1 to about 2, or about 1 to about 1. In embodiments, the composition of the present disclosure further comprises a nucleic acid encoding a donor comprising a transgene to be integrated, optionally wherein the transgene is defective or substantially absent in a disease state. In embodiments, the transgene comprises a cargo nucleic acid sequence and a first and a second donor end sequences. In embodiments, the cargo nucleic acid sequence is flanked by the first and the second donor end sequences.

In some embodiments, the donor comprises a gene encoding a complete polypeptide.

In some embodiments, the donor comprises a gene which is defective or substantially absent in a disease state.

In certain embodiments, the present disclosure provides a method for treating a disease or disorder ex vivo, comprising contacting a cell with the composition of the present disclosure or host cell of the present disclosure and administering the cell to a subject in need thereof.

In certain embodiments, the present disclosure provides a method for treating a disease or disorder in vivo, comprising administering the composition of the present disclosure or host cell of the present disclosure to a subject in need thereof.

### Transgene

In embodiments, the transgene is an exogenous wild-type gene that, e.g., corrects a defective function of one or more mutations in a recipient. For instance, in embodiments, the recipient may have a mutation that provides a disease phenotype (e.g., a defective or absent gene product). In embodiments, the donor system or method of the present disclosure provides a correction that restores the gene product and diminishes the disease phenotype.

In embodiments, the transgene is a gene that replaces, inactivates, or provides suicide or helper functions.

In embodiments, the transgene and/or disease to be treated is one or more of:
beta-thalassemia: BCL11a or β-globin or βA-T87Q-globin,
LCA: RPE65,
LHON: ND4,
Achromatopsia: CNGA3 or CNGA3/CNGB3,
Choroideremia: REP1,
PKD: RPK (Red cell PK),
Hemophilia: F8,
ADA-SCID: ADA,
Fabry disease: GLA,
MPS type I: IDUA, and
MPS type II: IDS.

In embodiments, the donor comprises a gene encoding a complete polypeptide. In embodiments, the donor comprises a gene which is defective or substantially absent in a disease state.

In embodiments, the transfecting of the cell is carried out using electroporation or calcium phosphate precipitation.

In embodiments, the transfecting of the cell is carried out using a lipid vehicle, optionally N-[1-(2,3-dioleoyloxy) propyl]-N,N,N-trimethylammonium chloride (DOTMA), 1,2-bis(oleoyloxy)-3-3-(trimethylammonia) propane (DOTAP), or 1,2-dioleoyl-3-dimethylammonium-propane (DODAP), dioleoylphosphatidylethanolamine (DOPE), cholesterol, LIPOFECTIN (cationic liposome formulation), LIPOFECTAMINE (cationic liposome formulation), LIPOFECTAMINE 2000 (cationic liposome formulation), LIPOFECTAMINE 3000 (cationic liposome formulation), TRANSFECTAM (cationic liposome formulation), a lipid nanoparticle, or a liposome and combinations thereof.

In embodiments, the transfecting of the cell is carried out using a lipid selected from one or more of the following categories: cationic lipids; anionic lipids; neutral lipids; multi-valent charged lipids; and zwitterionic lipids. In embodiments, a cationic lipid may be used to facilitate a charge-charge interaction with nucleic acids. In embodiments, the lipid is a neutral lipid. In embodiments, the neutral lipid is dioleoylphosphatidylethanolamine (DOPE), 1,2-Dioleoyl-sn-glycero-3-phosphocholine (DOPC), or cholesterol. In embodiments, cholesterol is derived from plant sources. In other embodiments, cholesterol is derived from animal, fungal, bacterial, or archaeal sources. In embodiments, the lipid is a cationic lipid. In embodiments, the cationic lipid is N-[1-(2,3-dioleoyloxy) propyl]-N,N,N-trimethylammonium chloride (DOTMA), 1,2-bis(oleoyloxy)-3-3-(trimethylammonia) propane (DOTAP), or 1,2-dioleoyl-3-dimethylammonium-propane (DODAP). In embodiments, one or more of the phospholipids 18:0 PC, 18:1 PC, 18:2 PC, DMPC, DSPE, DOPE, 18:2 PE, DMPE, or a combination thereof are used as lipids. In embodiments, the lipid is DOTMA and DOPE, optionally in a ratio of about 1:1. In embodiments, the lipid is DHDOS and DOPE, optionally in a ratio of about 1:1. In embodiments, the lipid is a commercially available product (e.g., LIPOFECTIN (cationic liposome formulation), LIPOFECTAMINE (cationic liposome formulation), LIPOFECTAMINE 2000 (cationic liposome formulation), LIPOFECTAMINE 3000 (cationic liposome formulation) (Life Technologies)).

In embodiments, the transfecting of the cell is carried out using a cationic vehicle, optionally LIPOFECTIN or TRANSFECTAM.

In embodiments, the transfecting of the cell is carried out using a lipid nanoparticle or a liposome.

In embodiments, the method is helper virus-free.

Epigenetic regulatory elements can be used to protect a transgene from unwanted epigenetic effects when placed near the transgene on a vector, including the transgene. See Ley et al., PloS One vol. 8,4 e62784. 30 Apr. 2013, doi:10.1371/journal.pone.0062784. For example, MARs were shown to increase genomic integration and integration of a transgene while preventing heterochromatin silencing, as exemplified by the human MAR1-68. See id.; see also Grandjean et al., Nucleic Acids Res. 2011 August; 39(15):e104. MARs can also act as insulators and thereby prevent the activation of neighboring cellular genes. Gaussin et al., Gene Ther. 2012 January; 19(1):15-24. It has been shown that a piggyBac donor containing human MARs in CHO cells mediated efficient and sustained expression from a few transgene copies, using cell populations generated without an antibiotic selection procedure. See Ley et al. (2013).

In embodiments, the cell is further transfected with a third nucleic acid having at least one chromatin element, wherein the at least one chromatin element is optionally a Matrix Attachment Region (MAR) element. MARs are expression-enhancing, epigenetic regulator elements which are used to enhance and/or facilitate transgene expression, as described, for example, in PCT/IB2010/002337 (WO2011033375), which is incorporated by reference herein in its entirety. A MAR element can be located in cis or trans to the transgene.

In embodiments, the transgene has a size of 100,000 bases or less, e.g., about 100,000 bases, or about 50,000 bases, or about 30,000 bases, or about 10,000 bases, or about 5,000 bases, or about 10,000 to about 100,000 bases, or about 30,000 to about 100,000 bases, or about 50,000 to about 100,000 bases, or about 10,000 to about 50,000 bases, or about 10,000 to about 30,000 bases, or about 30,000 to about 50,000 bases.

In embodiments, the transgene has a size of about 200,000 bases or less, e.g., about 200,000 bases, or about 10,000 to about 200,000 bases, or about 30,000 to about 200,000 bases, or about 50,000 to about 200,000 bases, or about 100,000 to about 200,000 bases, or about 150,000 to about 200,000 bases.

In embodiments, the insertion size of a gene is at least about 1,000 bases, or at least about 1,500 bases, or at least about 2,000 bases, or at least about 2,500 bases, or at least about 3,000 bases, or at least about 3,500 bases, or at least about 4,000 bases, or at least about 4,500 bases, or at least about 5,000 bases, or at least about 5,500 bases, or at least about 6,000 bases, or at least about 6,500 bases, or at least about 7,000 bases, or at least about 7,500 bases.

### Targeting Chimeric Constructs

In aspects, the present disclosure provides for a donor system, e.g., in embodiments, a helper enzyme comprises a targeting element.

In embodiments, the helper enzyme associated with the targeting element, is capable of inserting the donor comprising a transgene, optionally at a TA dinucleotide site or a TTAA (SEQ ID NO: 440) tetranucleotide site in a genomic safe harbor site (GSHS). In embodiments, the helper enzyme associated with the targeting element, is capable of inserting the donor comprising a transgene, optionally at a TA dinucleotide site or a ttTTAAaa (SEQ ID NO: 880) octanucleotide site in a genomic safe harbor site (GSHS). In embodiments, the helper enzyme associated with the targeting element, is capable of inserting the donor comprising a transgene, optionally at a TA dinucleotide site or a taTTAAta (SEQ ID NO: 881) octanucleotide site in a genomic safe harbor site (GSHS).

In embodiments, the helper enzyme associated with the targeting element has one or more mutations which confer hyperactivity.

In embodiments, the helper enzyme associated with the targeting element has gene cleavage (Exc) and/or gene integration (Int+) activity.

In embodiments, the helper enzyme associated with the targeting element has gene cleavage (Exc) and/or a lack of gene integration (Int−) activity.

In embodiments, the targeting element comprises one or more proteins or nucleic acids that are capable of binding to a nucleic acid.

In embodiments, the targeting element comprises one or more of a of a gRNA, optionally associated with a Cas enzyme, which is optionally catalytically inactive, transcription activator-like effector (TALE), Zinc finger, catalytically inactive transcription factor, nickase, a transcriptional activator, a transcriptional repressor, a recombinase, a DNA methyltransferase, a histone methyltransferase, and paternally expressed gene 10 (PEG10).

In embodiments, the targeting element comprises a transcription activator-like effector (TALE) DNA binding domain (DBD).

In embodiments, the TALE DBD comprises one or more repeat sequences. In embodiments, the TALE DBD comprises about 14, or about 15, or about, 16, or about 17, or about 18, or about 18.5 repeat sequences. In embodiments, the TALE DBD repeat sequences comprise 33 or 34 amino acids. In embodiments, the TALE DBD repeat sequences comprise a repeat variable di-residue (RVD) at residue 12 or 13 of the 33 or 34 amino acids. In embodiments, the RVD recognizes one base pair in the nucleic acid molecule. In embodiments, the RVD recognizes a C residue in the nucleic acid molecule and is selected from HD, N (gap), HA, ND, and HI. In embodiments, the RVD recognizes a G residue in the nucleic acid molecule and is selected from NN, NH, NK, HN, and NA. In embodiments, the RVD recognizes an A residue in the nucleic acid molecule and is selected from NI and NS. In embodiments, the RVD recognizes a T residue in the nucleic acid molecule and is selected from NG, HG, H (gap), and IG. In embodiments, the GSHS is in an open chromatin location in a chromosome. In embodiments, the GSHS is selected from adeno-associated virus site 1 (AAVS1), chemokine (C—C motif) receptor 5 (CCR5) gene, HIV-1 coreceptor, chromosome 4 GSHS, chromosome 22 GSHS and human Rosa26 locus. In embodiments, the GSHS is a located at the human Rosa26 locus. In embodiments, the GSHS is an adeno-associated virus site 1 (AAVS1).

In embodiments, the GSHS is located on human chromosome 2, 3, 4, 6, 10, 11, 17, 19 or 22. In embodiments, the GSHS is a located in intron 1 of a disease gene (e.g., HPRT, CFTR) or intron 1 of a disease gene (e.g., HPRT, CFTR). In embodiments, the targeting element is able to direct a transposition machinery to a GSHS or or intron 1 of a disease gene (e.g., HPRT, CFTR) of a nucleic acid molecule in a mammalian cell. In embodiments, the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

In embodiments, the targeting element comprises a Cas9 enzyme guide RNA complex. In embodiments, the Cas9 enzyme guide RNA complex comprises a nuclease-deficient dCas9 guide RNA complex. In embodiments, the targeting element comprises a Cas12 enzyme guide RNA complex. In embodiments, the targeting element comprises a nuclease-deficient dCas12 guide RNA complex, optionally dCas12j guide RNA complex or dCas12a guide RNA complex. In embodiments, the targeting element comprises a Cas12k enzyme guide RNA complex. In embodiments, the targeting element comprises a nuclease-deficient dCas12 guide RNA complex, optionally dCas12k guide RNA complex.

In embodiments, the targeting element comprises a CasX enzyme associated with a gRNA. In embodiments, the CasX enzyme associated with a gRNA comprises a catalytically inactive dCasx associated with a gRNA. In embodiments, the targeting element is or comprises a a CasX enzyme associated with a gRNA, optionally wherein the catalytic inactive dCasX.

In embodiments, a targeting chimeric system or construct, having a DBD fused to the helper enzyme directs binding of the helper to a specific sequence (e.g., transcription activator-like effector proteins (TALE) repeat variable di-residues (RVD) or gRNA) near a helper enzyme recognition site. The helper enzyme is thus prevented from binding to random recognition sites. In embodiments, the targeting chimeric construct binds to human GSHS. In embodiments, dCas9 (i.e., deficient for nuclease activity) is programmed with gRNAs directed to bind at a desired sequence of DNA in GSHS. In embodiments, TALEs described herein can physically sequester the helper enzyme to GSHS and promote transposition to nearby TTAA (SEQ ID NO: 440) sequences in close proximity to the RVD TALE nucleotide sequences. GSHS in open chromatin sites are specifically targeted based on the predilection for helpers to insert into open chromatin.

In embodiments, the helper enzyme is capable of targeted genomic integration by transposition is linked to or fused with a TALE DNA binding domain (DBD) or a Cas-based gene-editing system, such as, e.g., Cas9 or a variant thereof.

In embodiments, the targeting element targets the helper enzyme to a locus of interest. In embodiments, the targeting element comprises CRISPR (Clustered Regularly Interspaced Short Palindromic Repeat) associated protein 9 (Cas9), or a variant thereof. A CRISPR/Cas9 tool only requires Cas9 nuclease for DNA cleavage and a single-guide RNA (sgRNA) for target specificity. See Jinek et al. (2012) Science 337, 816-821; Chylinski et al. (2014) Nucleic Acids Res 42, 6091-6105. The inactivated form of Cas9, which is a nuclease-deficient (or inactive, or “catalytically dead” Cas9, is typically denoted as “dCas9,” has no substantial nuclease activity. Qi, L. S. et al. (2013). Cell 152, 1173-1183. CRISPR/dCas9 binds precisely to specific genomic sequences through targeting of guide RNA (gRNA) sequences. See Dominguez et al., Nat Rev Mol Cell Biol. 2016; 17:5-15; Wang et al., Annu Rev Biochem. 2016; 85:227-64. dCas9 is utilized to edit gene expression when applied to the transcription binding site of a desired site and/or locus in a genome. When the dCas9 protein is coupled to guide RNA (gRNA) to create dCas9 guide RNA complex, dCas9 prevents the proliferation of repeating codons and DNA sequences that might be harmful to an organism's genome. Essentially, when multiple repeat codons are produced, it elicits a response, or recruits an abundance of dCas9 to combat the overproduction of those codons and results in the shut-down of transcription. Thus, dCas9 works synergistically with gRNA and directly affects the DNA polymerase II from continuing transcription.

In embodiments, the targeting element comprises a nuclease-deficient Cas enzyme guide RNA complex. In embodiments, the targeting element comprises a nuclease-deficient (or inactive, or “catalytically dead” Cas, e.g., Cas9, typically denoted as “dCas” or “dCas9”) guide RNA complex.

In embodiments, the dCas9/gRNA complex comprises a guide RNA selected from: GTTTAGCTCACCCGTGAGCC (SEQ ID NO: 91), CCCAATATTATTGTTCTCTG (SEQ ID NO: 92), GGGGTGGGATAGGGGATACG (SEQ ID NO: 93), GGATCCCCCTCTACATTTAA (SEQ ID NO: 94), GTGATCTTGTACAAATCATT (SEQ ID NO: 95), CTACACAGAATCTGTTAGAA (SEQ ID NO: 96), TAAGCTAGAGAATAGATCTC (SEQ ID NO: 97), and TCAATACACTTAATGATTTA (SEQ ID NO: 98), wherein the guide RNA directs the helper enzyme to a chemokine (C—C motif) receptor 5 (CCR5) gene.

In embodiments, the dCas9/gRNA complex comprises a guide RNA selected from:

(SEQ ID NO: 99)
CACCGGGAGCCACGAAAACAGATCC;
(SEQ ID NO: 100)
CACCGCGAAAACAGATCCAGGGACA;
(SEQ ID NO: 101)
CACCGAGATCCAGGGACACGGTGCT;
(SEQ ID NO: 102)
CACCGGACACGGTGCTAGGACAGTG;
(SEQ ID NO: 103)
CACCGGAAAATGACCCAACAGCCTC;
(SEQ ID NO: 104)
CACCGGCCTGGCCGGCCTGACCACT;
(SEQ ID NO: 105)
CACCGCTGAGCACTGAAGGCCTGGC;
(SEQ ID NO: 106)
CACCGTGGTTTCCACTGAGCACTGA;
(SEQ ID NO: 107)
CACCGGATAGCCAGGAGTCCTTTCG;
(SEQ ID NO: 108)
CACCGGCGCTTCCAGTGCTCAGACT;
(SEQ ID NO: 109)
CACCGCAGTGCTCAGACTAGGGAAG;
(SEQ ID NO: 110)
CACCGGCCCCTCCTCCTTCAGAGCC;
(SEQ ID NO: 111)
CACCGTCCTTCAGAGCCAGGAGTCC;
(SEQ ID NO: 112)
CACCGTGGTTTCCGAGCTTGACCCT;
(SEQ ID NO: 113)
CACCGCTGCAGAGTATCTGCTGGGG;
(SEQ ID NO: 114)
CACCGCGTTCCTGCAGAGTATCTGC;
(SEQ ID NO: 115)
AAACGGATCTGTTTTCGTGGCTCCC;
(SEQ ID NO: 116)
AAACTGTCCCTGGATCTGTTTTCGC;
(SEQ ID NO: 117)
AAACAGCACCGTGTCCCTGGATCTC;
(SEQ ID NO: 118)
AAACCACTGTCCTAGCACCGTGTCC;
(SEQ ID NO: 119)
AAACGAGGCTGTTGGGTCATTTTCC;
(SEQ ID NO: 121)
AAACGCCAGGCCTTCAGTGCTCAGC;
(SEQ ID NO: 122)
AAACTCAGTGCTCAGTGGAAACCAC;
(SEQ ID NO: 123)
AAACCGAAAGGACTCCTGGCTATCC;
(SEQ ID NO: 124)
AAACAGTCTGAGCACTGGAAGCGCC;
(SEQ ID NO: 125)
AAACCTTCCCTAGTCTGAGCACTGC;
(SEQ ID NO: 126)
AAACGGCTCTGAAGGAGGAGGGGCC;
(SEQ ID NO: 127)
AAACGGACTCCTGGCTCTGAAGGAC;
(SEQ ID NO: 128)
AAACAGGGTCAAGCTCGGAAACCAC;
(SEQ ID NO: 129)
AAACGCAGATACTCTGCAGGAACGC;
(SEQ ID NO: 130)
AAACGCAGATACTCTGCAGGAACGC;
(SEQ ID NO: 131)
TCCCCTCCCAGAAAGACCTG;
(SEQ ID NO: 132)
TGGGCTCCAAGCAATCCTGG;
(SEQ ID NO: 133)
GTGGCTCAGGAGGTACCTGG;
(SEQ ID NO: 134)
GAGCCACGAAAACAGATCCA;
(SEQ ID NO: 135)
AAGTGAACGGGGAAGGGAGG;
(SEQ ID NO: 136)
GACAAAAGCCGAAGTCCAGG;
(SEQ ID NO: 137)
GTGGTTGATAAACCCACGTG;
(SEQ ID NO: 138)
TGGGAACAGCCACAGCAGGG;
(SEQ ID NO: 139)
GCAGGGGAACGGGGATGCAG;
(SEQ ID NO: 140)
GAGATGGTGGACGAGGAAGG;
(SEQ ID NO: 141)
GAGATGGCTCCAGGAAATGG;
(SEQ ID NO: 142)
TAAGGAATCTGCCTAACAGG;
(SEQ ID NO: 143)
TCAGGAGACTAGGAAGGAGG;
(SEQ ID NO: 144)
TATAAGGTGGTCCCAGCTCG;
(SEQ ID NO: 145)
CTGGAAGATGCCATGACAGG;
(SEQ ID NO: 146)
GCACAGACTAGAGAGGTAAG;
(SEQ ID NO: 147)
ACAGACTAGAGAGGTAAGGG;
(SEQ ID NO: 148)
GAGAGGTGACCCGAATCCAC;
(SEQ ID NO: 149)
GCACAGGCCCCAGAAGGAGA;
(SEQ ID NO: 150)
CCGGAGAGGACCCAGACACG;
(SEQ ID NO: 151)
GAGAGGACCCAGACACGGGG;
(SEQ ID NO: 152)
GCAACACAGCAGAGAGCAAG;
(SEQ ID NO: 153)
GAAGAGGGAGTGGAGGAAGA;
(SEQ ID NO: 154)
AAGACGGAACCTGAAGGAGG;
(SEQ ID NO: 155)
AGAAAGCGGCACAGGCCCAG;
(SEQ ID NO: 156)
GGGAAACAGTGGGCCAGAGG;
(SEQ ID NO: 157)
GTCCGGACTCAGGAGAGAGA;
(SEQ ID NO: 158)
GGCACAGCAAGGGCACTCGG;
(SEQ ID NO: 159)
GAAGAGGGGAAGTCGAGGGA;
(SEQ ID NO: 160)
GGGAATGGTAAGGAGGCCTG;
(SEQ ID NO: 161)
GCAGAGTGGTCAGCACAGAG;
(SEQ ID NO: 162)
GCACAGAGTGGCTAAGCCCA;
(SEQ ID NO: 163)
GACGGGGTGTCAGCATAGGG;
(SEQ ID NO: 164)
GCCCAGGGCCAGGAACGACG;
(SEQ ID NO: 165)
GGTGGAGTCCAGCACGGCGC;
(SEQ ID NO: 166)
ACAGGCCGCCAGGAACTCGG;
(SEQ ID NO: 167)
ACTAGGAAGTGTGTAGCACC;
(SEQ ID NO: 168)
ATGAATAGCAGACTGCCCCG;
(SEQ ID NO: 169)
ACACCCCTAAAAGCACAGTG;
(SEQ ID NO: 170)
CAAGGAGTTCCAGCAGGTGG;
(SEQ ID NO: 171)
AAGGAGTTCCAGCAGGTGGG;
(SEQ ID NO: 172)
TGGAAAGAGGAGGGAAGAGG;
(SEQ ID NO: 173)
TCGAATTCCTAACTGCCCCG;
(SEQ ID NO: 174)
GACCTGCCCAGCACACCCTG;
(SEQ ID NO: 175)
GGAGCAGCTGCGGCAGTGGG;
(SEQ ID NO: 176)
GGGAGGGAGAGCTTGGCAGG;
(SEQ ID NO: 177)
GTTACGTGGCCAAGAAGCAG;
(SEQ ID NO: 178)
GCTGAACAGAGAAGAGCTGG;
(SEQ ID NO: 179)
TCTGAGGGTGGAGGGACTGG;
(SEQ ID NO: 180)
GGAGAGGTGAGGGACTTGGG;
(SEQ ID NO: 181)
GTGAACCAGGCAGACAACGA;
(SEQ ID NO: 182)
CAGGTACCTCCTGAGCCACG;
(SEQ ID NO: 183)
GGGGGAGTAGGGGCATGCAG;
(SEQ ID NO: 184)
GCAAATGGCCAGCAAGGGTG;
(SEQ ID NO: 309)
CAAATGGCCAGCAAGGGTGG;
(SEQ ID NO: 310)
GCAGAACCTGAGGATATGGA;
(SEQ ID NO: 311)
AATACACAGAATGAAAATAG;
(SEQ ID NO: 312)
CTGGTGACTAGAATAGGCAG;
(SEQ ID NO: 313)
TGGTGACTAGAATAGGCAGT;
(SEQ ID NO: 314)
TAAAAGAATGTGAAAAGATG;
(SEQ ID NO: 315)
TCAGGAGTTCAAGACCACCC;
(SEQ ID NO: 316)
TGTAGTCCCAGTTATGCAGG;
(SEQ ID NO: 317)
GGGTTCACACCACAAATGCA;
(SEQ ID NO: 318)
GGCAAATGGCCAGCAAGGGT;
(SEQ ID NO: 319)
AGAAACCAATCCCAAAGCAA;
(SEQ ID NO: 320)
GCCAAGGACACCAAAACCCA;
(SEQ ID NO: 321)
AGTGGTGATAAGGCAACAGT;
(SEQ ID NO: 322)
CCTGAGACAGAAGTATTAAG;
(SEQ ID NO: 323)
AAGGTCACACAATGAATAGG;
(SEQ ID NO: 324)
CACCATACTAGGGAAGAAGA;
(SEQ ID NO: 327)
CAATACCCTGCCCTTAGTGG;
(SEQ ID NO: 325)
AATACCCTGCCCTTAGTGGG;
(SEQ ID NO: 326)
TTAGTGGGGGGTGGAGTGGG;
(SEQ ID NO: 328)
GTGGGGGGGGAGTGGGGGG;
(SEQ ID NO: 329)
GGGGGGTGGAGTGGGGGGTG;
(SEQ ID NO: 330)
GGGGTGGAGTGGGGGGTGGG;
(SEQ ID NO: 331)
GGGTGGAGTGGGGGGTGGGG;
(SEQ ID NO: 332)
GGGGGGGGGAAAGACATCG;
(SEQ ID NO: 333)
GCAGCTGTGAATTCTGATAG;
(SEQ ID NO: 334)
GAGATCAGAGAAACCAGATG;
(SEQ ID NO: 335)
TCTATACTGATTGCAGCCAG;
(SEQ ID NO: 185)
CACCGAATCGAGAAGCGACTCGACA;
(SEQ ID NO: 186)
CACCGGTCCCTGGGCGTTGCCCTGC;
(SEQ ID NO: 187)
CACCGCCCTGGGCGTTGCCCTGCAG;
(SEQ ID NO: 188)
CACCGCCGTGGGAAGATAAACTAAT;
(SEQ ID NO: 189)
CACCGTCCCCTGCAGGGCAACGCCC;
(SEQ ID NO: 190)
CACCGGTCGAGTCGCTTCTCGATTA;
(SEQ ID NO: 191)
CACCGCTGCTGCCTCCCGTCTTGTA;
(SEQ ID NO: 192)
CACCGGAGTGCCGCAATACCTTTAT;
(SEQ ID NO: 193)
CACCGACACTTTGGTGGTGCAGCAA;
(SEQ ID NO: 194)
CACCGTCTCAAATGGTATAAAACTC;
(SEQ ID NO: 195)
CACCGAATCCCGCCCATAATCGAGA;
(SEQ ID NO: 196)
CACCGTCCCGCCCATAATCGAGAAG;
(SEQ ID NO: 197)
CACCGCCCATAATCGAGAAGCGACT;
(SEQ ID NO: 198)
CACCGGAGAAGCGACTCGACATGGA;
(SEQ ID NO: 199)
CACCGGAAGCGACTCGACATGGAGG;
(SEQ ID NO: 200)
CACCGGCGACTCGACATGGAGGCGA;
(SEQ ID NO: 201)
AAACTGTCGAGTCGCTTCTCGATTC;
(SEQ ID NO: 202)
AAACGCAGGGCAACGCCCAGGGACC;
(SEQ ID NO: 203)
AAACCTGCAGGGCAACGCCCAGGGC;
(SEQ ID NO: 204)
AAACATTAGTTTATCTTCCCACGGC;
(SEQ ID NO: 205)
AAACGGGCGTTGCCCTGCAGGGGAC;
(SEQ ID NO: 206)
AAACTAATCGAGAAGCGACTCGACC;
(SEQ ID NO: 207)
AAACTACAAGACGGGAGGCAGCAGC;
(SEQ ID NO: 208)
AAACATAAAGGTATTGCGGCACTCC;
(SEQ ID NO: 209)
AAACTTGCTGCACCACCAAAGTGTC;
(SEQ ID NO: 210)
AAACGAGTTTTATACCATTTGAGAC;
(SEQ ID NO: 211)
AAACTCTCGATTATGGGCGGGATTC;
(SEQ ID NO: 212)
AAACCTTCTCGATTATGGGGGGGAC;
(SEQ ID NO: 213)
AAACAGTCGCTTCTCGATTATGGGC;
(SEQ ID NO: 214)
AAACTCCATGTCGAGTCGCTTCTCC;
(SEQ ID NO: 215)
AAACCCTCCATGTCGAGTCGCTTCC;
(SEQ ID NO: 216)
AAACTCGCCTCCATGTCGAGTCGCC;
(SEQ ID NO: 217)
CACCGACAGGGTTAATGTGAAGTCC;
(SEQ ID NO: 218)
CACCGTCCCCCTCTACATTTAAAGT;
(SEQ ID NO: 219)
CACCGCATTTAAAGTTGGTTTAAGT;
(SEQ ID NO: 220)
CACCGTTAGAAAATATAAAGAATAA;
(SEQ ID NO: 221)
CACCGTAAATGCTTACTGGTTTGAA;
(SEQ ID NO: 222)
CACCGTCCTGGGTCCAGAAAAAGAT;
(SEQ ID NO: 223)
CACCGTTGGGTGGTGAGCATCTGTG;
(SEQ ID NO: 224)
CACCGCGGGGAGAGTGGAGAAAAAG;
(SEQ ID NO: 225)
CACCGGTTAAAACTCTTTAGACAAC;
(SEQ ID NO: 226)
CACCGGAAAATCCCCACTAAGATCC;
(SEQ ID NO: 227)
AAACGGACTTCACATTAACCCTGTC;
(SEQ ID NO: 228)
AAACACTTTAAATGTAGAGGGGGAC;
(SEQ ID NO: 229)
AAACACTTAAACCAACTTTAAATGC;
(SEQ ID NO: 230)
AAACTTATTCTTTATATTTTCTAAC;
(SEQ ID NO: 231)
AAACTTCAAACCAGTAAGCATTTAC;
(SEQ ID NO: 232)
AAACATCTTTTTCTGGACCCAGGAC;
(SEQ ID NO: 233)
AAACCACAGATGCTCACCACCCAAC;
(SEQ ID NO: 234)
AAACCTTTTTCTCCACTCTCCCCGC;
(SEQ ID NO: 235)
AAACGTTGTCTAAAGAGTTTTAACC;
(SEQ ID NO: 236)
AAACGGATCTTAGTGGGGATTTTCC;
(SEQ ID NO: 237)
AGTAGCAGTAATGAAGCTGG;
(SEQ ID NO: 238)
ATACCCAGACGAGAAAGCTG;
(SEQ ID NO: 239)
TACCCAGACGAGAAAGCTGA;
(SEQ ID NO: 240)
GGTGGTGAGCATCTGTGTGG;
(SEQ ID NO: 241)
AAATGAGAAGAAGAGGCACA;
(SEQ ID NO: 242)
CTTGTGGCCTGGGAGAGCTG;
(SEQ ID NO: 243)
GCTGTAGAAGGAGACAGAGC;
(SEQ ID NO: 244)
GAGCTGGTTGGGAAGACATG;
(SEQ ID NO: 245)
CTGGTTGGGAAGACATGGGG;
(SEQ ID NO: 246)
CGTGAGGATGGGAAGGAGGG;
(SEQ ID NO: 247)
ATGCAGAGTCAGCAGAACTG;
(SEQ ID NO: 248)
AAGACATCAAGCACAGAAGG;
(SEQ ID NO: 249)
TCAAGCACAGAAGGAGGAGG;
(SEQ ID NO: 250)
AACCGTCAATAGGCAAAGGG;
(SEQ ID NO: 251)
CCGTATTTCAGACTGAATGG;
(SEQ ID NO: 252)
GAGAGGACAGGTGCTACAGG;
(SEQ ID NO: 253)
AACCAAGGAAGGGCAGGAGG;
(SEQ ID NO: 254)
GACCTCTGGGTGGAGACAGA;
(SEQ ID NO: 255)
CAGATGACCATGACAAGCAG;
(SEQ ID NO: 256)
AACACCAGTGAGTAGAGCGG;
(SEQ ID NO: 257)
AGGACCTTGAAGCACAGAGA;
(SEQ ID NO: 258)
TACAGAGGCAGACTAACCCA;
(SEQ ID NO: 259)
ACAGAGGCAGACTAACCCAG;
(SEQ ID NO: 260)
TAAATGACGTGCTAGACCTG;
(SEQ ID NO: 261)
AGTAACCACTCAGGACAGGG;
(SEQ ID NO: 262)
ACCACAAAACAGAAACACCA;
(SEQ ID NO: 263)
GTTTGAAGACAAGCCTGAGG;
(SEQ ID NO: 264)
GCTGAACCCCAAAAGACAGG;
(SEQ ID NO: 265)
GCAGCTGAGACACACACCAG;
(SEQ ID NO: 266)
AGGACACCCCAAAGAAGCTG;
(SEQ ID NO: 267)
GGACACCCCAAAGAAGCTGA;
(SEQ ID NO: 268)
CCAGTGCAATGGACAGAAGA;
(SEQ ID NO: 269)
AGAAGAGGGAGCCTGCAAGT;
(SEQ ID NO: 270)
GTGTTTGGGCCCTAGAGCGA;
(SEQ ID NO: 271)
CATGTGCCTGGTGCAATGCA;
(SEQ ID NO: 272)
TACAAAGAGGAAGATAAGTG;
(SEQ ID NO: 273)
GTCACAGAATACACCACTAG;
(SEQ ID NO: 274)
GGGTTACCCTGGACATGGAA;
(SEQ ID NO: 275)
CATGGAAGGGTATTCACTCG;
(SEQ ID NO: 276)
AGAGTGGCCTAGACAGGCTG;
(SEQ ID NO: 277)
CATGCTGGACAGCTCGGCAG;
(SEQ ID NO: 278)
AGTGAAAGAAGAGAAAATTC;
(SEQ ID NO: 279)
TGGTAAGTCTAAGAAACCTA;
(SEQ ID NO: 280)
CCCACAGCCTAACCACCCTA;
(SEQ ID NO: 281)
AATATTTCAAAGCCCTAGGG;
(SEQ ID NO: 282)
GCACTCGGAACAGGGTCTGG;
(SEQ ID NO: 283)
AGATAGGAGCTCCAACAGTG;
(SEQ ID NO: 284)
AAGTTAGAGCAGCCAGGAAA;
(SEQ ID NO: 285)
TAGAGCAGCCAGGAAAGGGA;
(SEQ ID NO: 286)
TGAATACCCTTCCATGTCCA;
(SEQ ID NO: 287)
CCTGCATTGCACCAGGCACA;
(SEQ ID NO: 288)
TCTAGGGCCCAAACACACCT;
(SEQ ID NO: 289)
TCCCTCCATCTATCAAAAGG;
(SEQ ID NO: 290)
AGCCCTGAGACAGAAGCAGG;
(SEQ ID NO: 291)
GCCCTGAGACAGAAGCAGGT;
(SEQ ID NO: 292)
AGGAGATGCAGTGATACGCA;
(SEQ ID NO: 293)
ACAATACCAAGGGTATCCGG;
(SEQ ID NO: 294)
TGATAAAGAAAACAAAGTGA;
(SEQ ID NO: 295)
AAAGAAAACAAAGTGAGGGA;
(SEQ ID NO: 296)
GTGGCAAGTGGAGAAATTGA;
(SEQ ID NO: 297)
CAAGTGGAGAAATTGAGGGA;
(SEQ ID NO: 298)
GTGGTGATGATTGCAGCTGG;
(SEQ ID NO: 299)
CTATGTGCCTGACACACAGG;
(SEQ ID NO: 300)
GGGTTGGACCAGGAAAGAGG;
(SEQ ID NO: 301)
GATGCCTGGAAAAGGAAAGA;
(SEQ ID NO: 302)
TAGTATGCACCTGCAAGAGG;
(SEQ ID NO: 303)
TATGCACCTGCAAGAGGCGG;
(SEQ ID NO: 304)
AGGGGAAGAAGAGAAGCAGA;
(SEQ ID NO: 305)
GCTGAATCAAGAGACAAGCG;
(SEQ ID NO: 306)
AAGCAAATAAATCTCCTGGG;
(SEQ ID NO: 307)
AGATGAGTGCTAGAGACTGG;
and
(SEQ ID NO: 308)
CTGATGGTTGAGCACAGCAG.

In embodiments, the guide RNAs are: AATCGAGAAGCGACTCGACA (SEQ ID NO: 425), and tgccctgcaggggagtgagc (SEQ ID NO: 426). In embodiments, the guide RNAs are gaagcgactcgacatggagg (SEQ ID NO: 427) and cctgcaggggagtgagcagc (SEQ ID NO: 428).

In embodiments, guide RNAs (gRNAs) for targeting human genomic safe harbor sites using any of the gRNA-based targeting elements, e.g., without limitation dCas, in areas of open chromatin are as shown in TABLE 19.

TABLE 19

| GSHS | IDENTIFIER | SEQUENCE |
|---|---|---|
| AAVS1 | 14F | GGAGCCACGAAAACAGATCC (SEQ ID NO: 800) |
| AAVS1 | 15F | CGAAAACAGATCCAGGGACA (SEQ ID NO: 801) |
| AAVS1 | 16F | AGATCCAGGGACACGGTGCT (SEQ ID NO: 802) |
| AAVS1 | 17F | GACACGGTGCTAGGACAGTG (SEQ ID NO: 803) |
| AAVS1 | 18F | GAAAATGACCCAACAGCCTC (SEQ ID NO: 804) |
| AAVS1 | 19F | GCCTGGCCGGCCTGACCACT (SEQ ID NO: 805) |
| AAVS1 | 20F | CTGAGCACTGAAGGCCTGGC (SEQ ID NO: 806) |
| AAVS1 | 21F | TGGTTTCCACTGAGCACTGA (SEQ ID NO: 807) |
| AAVS1 | 22F | GATAGCCAGGAGTCCTTTCG (SEQ ID NO: 808) |
| AAVS1 | 23F | GCGCTTCCAGTGCTCAGACT (SEQ ID NO: 809) |
| AAVS1 | 24F | CAGTGCTCAGACTAGGGAAG (SEQ ID NO: 810) |
| AAVS1 | 25F | GCCCCTCCTCCTTCAGAGCC (SEQ ID NO: 811) |
| AAVS1 | 26F | TCCTTCAGAGCCAGGAGTCC (SEQ ID NO: 812) |
| AAVS1 | 27F | TGGTTTCCGAGCTTGACCCT (SEQ ID NO: 813) |
| AAVS1 | 28F | CTGCAGAGTATCTGCTGGGG (SEQ ID NO: 814) |
| AAVS1 | 29F | CGTTCCTGCAGAGTATCTGC (SEQ ID NO: 815) |
| AAVS1 | AAVS1 | TCCCCTCCCAGAAAGACCTG (SEQ ID NO: 131) |
| AAVS1 | GAAVS2 | TGGGCTCCAAGCAATCCTGG (SEQ ID NO: 132) |
| AAVS1 | GAAVS3 | GTGGCTCAGGAGGTACCTGG (SEQ ID NO: 133) |
| AAVS1 | GAAVS4 | GAGCCACGAAAACAGATCCA (SEQ ID NO: 134) |
| AAVS1 | GAAVS5 | AAGTGAACGGGGAAGGGAGG (SEQ ID NO: 135) |
| AAVS1 | GAAVS6 | GACAAAAGCCGAAGTCCAGG (SEQ ID NO: 136) |
| AAVS1 | GAAVS7 | GTGGTTGATAAACCCACGTG (SEQ ID NO: 137) |
| AAVS1 | GAAVS8 | TGGGAACAGCCACAGCAGGG (SEQ ID NO: 138) |
| AAVS1 | GAAVS9 | GCAGGGGAACGGGGATGCAG (SEQ ID NO: 139) |
| AAVS1 | GAAVS10 | GAGATGGTGGACGAGGAAGG (SEQ ID NO: 140) |
| AAVS1 | GAAVS11 | GAGATGGCTCCAGGAAATGG (SEQ ID NO: 141) |
| AAVS1 | GAAVS12 | TAAGGAATCTGCCTAACAGG (SEQ ID NO: 142) |
| AAVS1 | GAAVS13 | TCAGGAGACTAGGAAGGAGG (SEQ ID NO: 143) |
| AAVS1 | GAAVS14 | TATAAGGTGGTCCCAGCTCG (SEQ ID NO: 144) |
| AAVS1 | GAAVS15 | CTGGAAGATGCCATGACAGG (SEQ ID NO: 145) |
| AAVS1 | GAAVS16 | GCACAGACTAGAGAGGTAAG (SEQ ID NO: 146) |
| AAVS1 | GAAVS17 | ACAGACTAGAGAGGTAAGGG (SEQ ID NO: 147) |
| AAVS1 | GAAVS18 | GAGAGGTGACCCGAATCCAC (SEQ ID NO: 148) |
| AAVS1 | GAAVS19 | GCACAGGCCCCAGAAGGAGA (SEQ ID NO: 149) |
| AAVS1 | GAAVS20 | CCGGAGAGGACCCAGACACG (SEQ ID NO: 150) |
| AAVS1 | GAAVS21 | GAGAGGACCCAGACACGGGG (SEQ ID NO: 151) |
| AAVS1 | GAAVS22 | GCAACACAGCAGAGAGCAAG (SEQ ID NO: 152) |
| AAVS1 | GAAVS23 | GAAGAGGGAGTGGAGGAAGA (SEQ ID NO: 153) |
| AAVS1 | GAAVS24 | AAGACGGAACCTGAAGGAGG (SEQ ID NO: 154) |
| AAVS1 | GAAVS25 | AGAAAGCGGCACAGGCCCAG (SEQ ID NO: 155) |
| AAVS1 | GAAVS26 | GGGAAACAGTGGGCCAGAGG (SEQ ID NO: 156) |
| AAVS1 | GAAVS27 | GTCCGGACTCAGGAGAGAGA (SEQ ID NO: 157) |
| AAVS1 | GAAVS28 | GGCACAGCAAGGGCACTCGG (SEQ ID NO: 158) |
| AAVS1 | GAAVS29 | GAAGAGGGGAAGTCGAGGGA (SEQ ID NO: 159) |
| AAVS1 | GAAVS30 | GGGAATGGTAAGGAGGCCTG (SEQ ID NO: 160) |
| AAVS1 | GAAVS31 | GCAGAGTGGTCAGCACAGAG (SEQ ID NO: 161) |
| AAVS1 | GAAVS32 | GCACAGAGTGGCTAAGCCCA (SEQ ID NO: 162) |
| AAVS1 | GAAVS33 | GACGGGGTGTCAGCATAGGG (SEQ ID NO: 163) |
| AAVS1 | GAAVS34 | GCCCAGGGCCAGGAACGACG (SEQ ID NO: 164) |
| AAVS1 | GAAVS35 | GGTGGAGTCCAGCACGGCGC (SEQ ID NO: 165) |
| AAVS1 | GAAVS36 | ACAGGCCGCCAGGAACTCGG (SEQ ID NO: 166) |
| AAVS1 | GAAVS37 | ACTAGGAAGTGTGTAGCACC (SEQ ID NO: 167) |
| AAVS1 | GAAVS38 | ATGAATAGCAGACTGCCCCG (SEQ ID NO: 168) |
| AAVS1 | GAAVS39 | ACACCCCTAAAAGCACAGTG (SEQ ID NO: 169) |
| AAVS1 | GAAVS40 | CAAGGAGTTCCAGCAGGTGG (SEQ ID NO: 170) |
| AAVS1 | GAAVS41 | AAGGAGTTCCAGCAGGTGGG (SEQ ID NO: 171) |
| AAVS1 | GAAVS42 | TGGAAAGAGGAGGGAAGAGG (SEQ ID NO: 172) |
| AAVS1 | GAAVS43 | TCGAATTCCTAACTGCCCCG (SEQ ID NO: 173) |
| AAVS1 | GAAVS44 | GACCTGCCCAGCACACCCTG (SEQ ID NO: 174) |
| AAVS1 | GAAVS45 | GGAGCAGCTGCGGCAGTGGG (SEQ ID NO: 175) |
| AAVS1 | GAAVS46 | GGGAGGGAGAGCTTGGCAGG (SEQ ID NO: 176) |
| AAVS1 | GAAVS47 | GTTACGTGGCCAAGAAGCAG (SEQ ID NO: 177) |
| AAVS1 | GAAVS48 | GCTGAACAGAGAAGAGCTGG (SEQ ID NO: 178) |
| AAVS1 | GAAVS49 | TCTGAGGGTGGAGGGACTGG (SEQ ID NO: 179) |
| AAVS1 | GAAVS50 | GGAGAGGTGAGGGACTTGGG (SEQ ID NO: 180) |
| AAVS1 | GAAVS51 | GTGAACCAGGCAGACAACGA (SEQ ID NO: 181) |
| AAVS1 | GAAVS52 | CAGGTACCTCCTGAGCCACG (SEQ ID NO: 182) |
| AAVS1 | GAAVS53 | GGGGGAGTAGGGGCATGCAG (SEQ ID NO: 183) |
| HROSA26 | GHROSA26-1 | GCAAATGGCCAGCAAGGGTG (SEQ ID NO: 184) |
| HROSA26 | GHROSA26-2 | CAAATGGCCAGCAAGGGTGG (SEQ ID NO: 309) |
| HROSA26 | GHROSA26-3 | GCAGAACCTGAGGATATGGA (SEQ ID NO: 310) |
| HROSA26 | GHROSA26-3 | AATACACAGAATGAAAATAG (SEQ ID NO: 311) |
| HROSA26 | GHROSA26-4 | CTGGTGACTAGAATAGGCAG (SEQ ID NO: 312) |
| HROSA26 | GHROSA26-5 | TGGTGACTAGAATAGGCAGT (SEQ ID NO: 313) |
| HROSA26 | GHROSA26-6 | TAAAAGAATGTGAAAAGATG (SEQ ID NO: 314) |
| HROSA26 | GHROSA26-7 | TCAGGAGTTCAAGACCACCC (SEQ ID NO: 315) |
| HROSA26 | GHROSA26-8 | TGTAGTCCCAGTTATGCAGG (SEQ ID NO: 316) |
| HROSA26 | GHROSA26-9 | GGGTTCACACCACAAATGCA (SEQ ID NO: 317) |
| HROSA26 | GHROSA26-10 | GGCAAATGGCCAGCAAGGGT (SEQ ID NO: 318) |
| HROSA26 | GHROSA26-11 | AGAAACCAATCCCAAAGCAA (SEQ ID NO: 319) |
| HROSA26 | GHROSA26-12 | GCCAAGGACACCAAAACCCA (SEQ ID NO: 320) |
| HROSA26 | GHROSA26-13 | AGTGGTGATAAGGCAACAGT (SEQ ID NO: 321) |
| HROSA26 | GHROSA26-14 | CCTGAGACAGAAGTATTAAG (SEQ ID NO: 322) |
| HROSA26 | GHROSA26-15 | AAGGTCACACAATGAATAGG (SEQ ID NO: 323) |
| HROSA26 | GHROSA26-16 | CACCATACTAGGGAAGAAGA (SEQ ID NO: 324) |
| HROSA26 | GHROSA26-17 | CAATACCCTGCCCTTAGTGG (SEQ ID NO: 327) |
| HROSA26 | GHROSA26-18 | AATACCCTGCCCTTAGTGGG (SEQ ID NO: 325) |
| HROSA26 | GHROSA26-19 | TTAGTGGGGGGTGGAGTGGG (SEQ ID NO: 326) |
| HROSA26 | GHROSA26-20 | GTGGGGGGTGGAGTGGGGGG (SEQ ID NO: 328) |
| HROSA26 | GHROSA26-21 | GGGGGGTGGAGTGGGGGGTG (SEQ ID NO: 329) |
| HROSA26 | GHROSA26-22 | GGGGTGGAGTGGGGGGTGGG (SEQ ID NO: 330) |
| HROSA26 | GHROSA26-23 | GGGTGGAGTGGGGGGTGGGG (SEQ ID NO: 331) |
| HROSA26 | GHROSA26-24 | GGGGGTGGGGAAAGACATCG (SEQ ID NO: 332) |
| HROSA26 | GHROSA26-25 | GCAAATGGCCAGCAAGGGTG (SEQ ID NO: 184) |
| HROSA26 | GHROSA26-26 | CAAATGGCCAGCAAGGGTGG (SEQ ID NO: 309) |
| HROSA26 | GHROSA26-27 | GCAGAACCTGAGGATATGGA (SEQ ID NO: 310) |
| HROSA26 | GHROSA26-28 | AATACACAGAATGAAAATAG (SEQ ID NO: 311) |
| HROSA26 | GHROSA26-29 | CTGGTGACTAGAATAGGCAG (SEQ ID NO: 312) |
| HROSA26 | GHROSA26-30 | TGGTGACTAGAATAGGCAGT (SEQ ID NO: 313) |
| HROSA26 | GHROSA26-31 | TAAAAGAATGTGAAAAGATG (SEQ ID NO: 314) |
| HROSA26 | GHROSA26-32 | TCAGGAGTTCAAGACCACCC (SEQ ID NO: 315) |
| HROSA26 | GHROSA26-33 | TGTAGTCCCAGTTATGCAGG (SEQ ID NO: 316) |
| HROSA26 | GHROSA26-34 | GGGTTCACACCACAAATGCA (SEQ ID NO: 317) |
| HROSA26 | GHROSA26-35 | GGCAAATGGCCAGCAAGGGT (SEQ ID NO: 318) |
| HROSA26 | GHROSA26-36 | AGAAACCAATCCCAAAGCAA (SEQ ID NO: 319) |
| HROSA26 | GHROSA26-37 | GCCAAGGACACCAAAACCCA (SEQ ID NO: 320) |
| HROSA26 | GHROSA26-38 | AGTGGTGATAAGGCAACAGT (SEQ ID NO: 321) |
| HROSA26 | GHROSA26-39 | CCTGAGACAGAAGTATTAAG (SEQ ID NO: 322) |
| HROSA26 | GHROSA26-40 | AAGGTCACACAATGAATAGG (SEQ ID NO: 323) |
| HROSA26 | GHROSA26-41 | CACCATACTAGGGAAGAAGA (SEQ ID NO: 324) |
| HROSA26 | GHROSA26-42 | CAATACCCTGCCCTTAGTGG (SEQ ID NO: 327) |
| HROSA26 | GHROSA26-43 | AATACCCTGCCCTTAGTGGG (SEQ ID NO: 325) |
| HROSA26 | GHROSA26-44 | TTAGTGGGGGGTGGAGTGGG (SEQ ID NO: 326) |
| HROSA26 | GHROSA26-45 | GTGGGGGGTGGAGTGGGGGG (SEQ ID NO: 328) |
| HROSA26 | GHROSA26-46 | GGGGGGTGGAGTGGGGGGTG (SEQ ID NO: 329) |
| HROSA26 | GHROSA26-47 | GGGGTGGAGTGGGGGGTGGG (SEQ ID NO: 330) |
| HROSA26 | GHROSA26-48 | GGGTGGAGTGGGGGGTGGGG (SEQ ID NO: 331) |
| HROSA26 | GHROSA26-49 | GGGGGTGGGGAAAGACATCG (SEQ ID NO: 332) |
| HROSA26 | GHROSA26-50 | GCAGCTGTGAATTCTGATAG (SEQ ID NO: 333) |
| HROSA26 | GHROSA26-51 | GAGATCAGAGAAACCAGATG (SEQ ID NO: 334) |
| HROSA26 | GHROSA26-52 | TCTATACTGATTGCAGCCAG (SEQ ID NO: 335) |
| HROSA26 | GHROSA26-1 | GCAAATGGCCAGCAAGGGTG (SEQ ID NO: 184) |
| HROSA26 | 44F | AATCGAGAAGCGACTCGACA (SEQ ID NO: 185) |
| HROSA26 | 45F | GTCCCTGGGCGTTGCCCTGC (SEQ ID NO: 186) |
| HROSA26 | 46F | CCCTGGGCGTTGCCCTGCAG (SEQ ID NO: 187) |
| HROSA26 | 1NF | CCGTGGGAAGATAAACTAAT (SEQ ID NO: 188) |
| HROSA26 | 2NE | TCCCCTGCAGGGCAACGCCC (SEQ ID NO: 189) |
| HROSA26 | 3NF | GTCGAGTCGCTTCTCGATTA (SEQ ID NO: 190) |
| HROSA26 | 4NF | CTGCTGCCTCCCGTCTTGTA (SEQ ID NO: 191) |
| HROSA26 | 5NE | GAGTGCCGCAATACCTTTAT (SEQ ID NO: 192) |
| HROSA26 | 6NE | ACACTTTGGTGGTGCAGCAA (SEQ ID NO: 193) |
| HROSA26 | 7NE | TCTCAAATGGTATAAAACTC (SEQ ID NO: 194) |
| HROSA26 | 8NF | CCGTGGGAAGATAAACTAAT (SEQ ID NO: 188) |
| HROSA26 | 9F | AATCCCGCCCATAATCGAGA (SEQ ID NO: 195) |
| HROSA26 | 10F | TCCCGCCCATAATCGAGAAG (SEQ ID NO: 196) |
| HROSA26 | 11F | CCCATAATCGAGAAGCGACT (SEQ ID NO: 197) |
| HROSA26 | 12F | GAGAAGCGACTCGACATGGA (SEQ ID NO: 198) |
| HROSA26 | 13F | GAAGCGACTCGACATGGAGG (SEQ ID NO: 199) |
| HROSA26 | 14F | GCGACTCGACATGGAGGCGA (SEQ ID NO: 200) |
| HROSA26 | 44F | AAACTGTCGAGTCGCTTCTCGATTC (SEQ ID NO: 201) |
| HROSA26 | 45F | AAACGCAGGGCAACGCCCAGGGACC (SEQ ID NO: 202) |
| HROSA26 | 46F | AAACCTGCAGGGCAACGCCCAGGGC (SEQ ID NO: 203) |
| CCR5 | 1F | ACAGGGTTAATGTGAAGTCC (SEQ ID NO: 217) |
| CCR5 | 2F | TCCCCCTCTACATTTAAAGT (SEQ ID NO: 218) |
| CCR5 | 3F | CATTTAAAGTTGGTTTAAGT (SEQ ID NO: 219) |
| CCR5 | 4F | TTAGAAAATATAAAGAATAA (SEQ ID NO: 220) |
| CCR5 | 5 | TAAATGCTTACTGGTTTGAA (SEQ ID NO: 221) |
| CCR5 | 6F | TCCTGGGTCCAGAAAAAGAT (SEQ ID NO: 222) |
| CCR5 | 7F | TTGGGTGGTGAGCATCTGTG (SEQ ID NO: 223) |
| CCR5 | 8F | CGGGGAGAGTGGAGAAAAAG (SEQ ID NO: 224) |
| CCR5 | 9F | GTTAAAACTCTTTAGACAAC (SEQ ID NO: 225) |
| CCR5 | 10F | GAAAATCCCCACTAAGATCC (SEQ ID NO: 226) |
| CCR5 | GCCR5-1 | AGTAGCAGTAATGAAGCTGG (SEQ ID NO: 237) |
| CCR5 | GCCR5-2 | ATACCCAGACGAGAAAGCTG (SEQ ID NO: 238) |
| CCR5 | GCCR5-3 | TACCCAGACGAGAAAGCTGA (SEQ ID NO: 239) |
| CCR5 | GCCR5-4 | GGTGGTGAGCATCTGTGTGG (SEQ ID NO: 240) |
| CCR5 | GCCR5-5 | AAATGAGAAGAAGAGGCACA (SEQ ID NO: 241) |
| CCR5 | GCCR5-6 | CTTGTGGCCTGGGAGAGCTG (SEQ ID NO: 242) |
| CCR5 | GCCR5-7 | GCTGTAGAAGGAGACAGAGC (SEQ ID NO: 243) |
| CCR5 | GCCR5-8 | GAGCTGGTTGGGAAGACATG (SEQ ID NO: 244) |
| CCR5 | GCCR5-9 | CTGGTTGGGAAGACATGGGG (SEQ ID NO: 245) |
| CCR5 | GCCR5-10 | CGTGAGGATGGGAAGGAGGG (SEQ ID NO: 246) |
| CCR5 | GCCR5-11 | ATGCAGAGTCAGCAGAACTG (SEQ ID NO: 247) |
| CCR5 | GCCR5-12 | AAGACATCAAGCACAGAAGG (SEQ ID NO: 248) |
| CCR5 | GCCR5-13 | TCAAGCACAGAAGGAGGAGG (SEQ ID NO: 249) |
| CCR5 | GCCR5-14 | AACCGTCAATAGGCAAAGGG (SEQ ID NO: 250) |
| CCR5 | GCCR5-15 | CCGTATTTCAGACTGAATGG (SEQ ID NO: 251) |
| CCR5 | GCCR5-16 | GAGAGGACAGGTGCTACAGG (SEQ ID NO: 252) |
| CCR5 | GCCR5-17 | AACCAAGGAAGGGCAGGAGG (SEQ ID NO: 253) |
| CCR5 | GCCR5-18 | GACCTCTGGGTGGAGACAGA (SEQ ID NO: 254) |
| CCR5 | GCCR5-19 | CAGATGACCATGACAAGCAG (SEQ ID NO: 255) |
| CCR5 | GCCR5-20 | AACACCAGTGAGTAGAGCGG (SEQ ID NO: 256) |
| CCR5 | GCCR5-21 | AGGACCTTGAAGCACAGAGA (SEQ ID NO: 257) |
| CCR5 | GCCR5-22 | TACAGAGGCAGACTAACCCA (SEQ ID NO: 258) |
| CCR5 | GCCR5-23 | ACAGAGGCAGACTAACCCAG (SEQ ID NO: 259) |
| CCR5 | GCCR5-24 | TAAATGACGTGCTAGACCTG (SEQ ID NO: 260) |
| CCR5 | GCCR5-25 | AGTAACCACTCAGGACAGGG (SEQ ID NO: 261) |
| CHR2 | GCHR2-1 | ACCACAAAACAGAAACACCA (SEQ ID NO: 262) |
| CHR2 | GCHR2-2 | GTTTGAAGACAAGCCTGAGG (SEQ ID NO: 263) |
| CHR4 | GCHR4-1 | GCTGAACCCCAAAAGACAGG (SEQ ID NO: 264) |
| CHR4 | GCHR4-2 | GCAGCTGAGACACACACCAG (SEQ ID NO: 265) |
| CHR4 | GCHR4-3 | AGGACACCCCAAAGAAGCTG (SEQ ID NO: 266) |
| CHR4 | GCHR4-4 | GGACACCCCAAAGAAGCTGA (SEQ ID NO: 267) |
| CHR6 | GCHR6-1 | CCAGTGCAATGGACAGAAGA (SEQ ID NO: 268) |
| CHR6 | GCHR6-2 | AGAAGAGGGAGCCTGCAAGT (SEQ ID NO: 269) |
| CHR6 | GCHR6-3 | GTGTTTGGGCCCTAGAGCGA (SEQ ID NO: 270) |
| CHR6 | GCHR6-4 | CATGTGCCTGGTGCAATGCA (SEQ ID NO: 271) |
| CHR6 | GCHR6-5 | TACAAAGAGGAAGATAAGTG (SEQ ID NO: 272) |
| CHR6 | GCHR6-6 | GTCACAGAATACACCACTAG (SEQ ID NO: 273) |
| CHR6 | GCHR6-7 | GGGTTACCCTGGACATGGAA (SEQ ID NO: 274) |
| CHR6 | GCHR6-8 | CATGGAAGGGTATTCACTCG (SEQ ID NO: 275) |
| CHR6 | GCHR6-9 | AGAGTGGCCTAGACAGGCTG (SEQ ID NO: 276) |
| CHR6 | GCHR6-10 | CATGCTGGACAGCTCGGCAG (SEQ ID NO: 277) |
| CHR6 | GCHR6-11 | AGTGAAAGAAGAGAAAATTC (SEQ ID NO: 278) |
| CHR6 | GCHR6-12 | TGGTAAGTCTAAGAAACCTA (SEQ ID NO: 279) |
| CHR6 | GCHR6-13 | CCCACAGCCTAACCACCCTA (SEQ ID NO: 280) |
| CHR6 | GCHR6-14 | AATATTTCAAAGCCCTAGGG (SEQ ID NO: 281) |
| CHR6 | GCHR6-15 | GCACTCGGAACAGGGTCTGG (SEQ ID NO: 282) |
| CHR6 | GCHR6-16 | AGATAGGAGCTCCAACAGTG (SEQ ID NO: 283) |
| CHR6 | GCHR6-17 | AAGTTAGAGCAGCCAGGAAA (SEQ ID NO: 284) |
| CHR6 | GCHR6-18 | TAGAGCAGCCAGGAAAGGGA (SEQ ID NO: 285) |
| CHR6 | GCHR6-19 | TGAATACCCTTCCATGTCCA (SEQ ID NO: 286) |
| CHR6 | GCHR6-20 | CCTGCATTGCACCAGGCACA (SEQ ID NO: 287) |
| CHR6 | GCHR6-21 | TCTAGGGCCCAAACACACCT (SEQ ID NO: 288) |
| CHR6 | GCHR6-22 | TCCCTCCATCTATCAAAAGG (SEQ ID NO: 289) |
| CHR10 | GCHR10-1 | AGCCCTGAGACAGAAGCAGG (SEQ ID NO: 290) |
| CHR10 | GCHR10-2 | GCCCTGAGACAGAAGCAGGT (SEQ ID NO: 291) |
| CHR10 | GCHR10-3 | AGGAGATGCAGTGATACGCA (SEQ ID NO: 292) |
| CHR10 | GCHR10-4 | ACAATACCAAGGGTATCCGG (SEQ ID NO: 293) |
| CHR10 | GCHR10-5 | TGATAAAGAAAACAAAGTGA (SEQ ID NO: 294) |
| CHR10 | GCHR10-6 | AAAGAAAACAAAGTGAGGGA (SEQ ID NO: 295) |
| CHR10 | GCHR10-7 | GTGGCAAGTGGAGAAATTGA (SEQ ID NO: 296) |
| CHR10 | GCHR10-8 | CAAGTGGAGAAATTGAGGGA (SEQ ID NO: 297) |
| CHR10 | GCHR10-9 | GTGGTGATGATTGCAGCTGG (SEQ ID NO: 298) |
| CHR11 | GCHR11-1 | CTATGTGCCTGACACACAGG (SEQ ID NO: 299) |
| CHR11 | GCHR11-2 | GGGTTGGACCAGGAAAGAGG (SEQ ID NO: 300) |
| CHR17 | GCHR17-1 | GATGCCTGGAAAAGGAAAGA (SEQ ID NO: 301) |
| CHR17 | GCHR17-2 | TAGTATGCACCTGCAAGAGG (SEQ ID NO: 302) |
| CHR17 | GCHR17-3 | TATGCACCTGCAAGAGGCGG (SEQ ID NO: 303) |
| CHR17 | GCHR17-4 | AGGGGAAGAAGAGAAGCAGA (SEQ ID NO: 304) |
| CHR17 | GCHR17-5 | GCTGAATCAAGAGACAAGCG (SEQ ID NO: 305) |
| CHR17 | GCHR17-6 | AAGCAAATAAATCTCCTGGG (SEQ ID NO: 306) |
| CHR17 | GCHR17-7 | AGATGAGTGCTAGAGACTGG (SEQ ID NO: 307) |
| CHR17 | GCHR17-8 | CTGATGGTTGAGCACAGCAG (SEQ ID NO: 308) |

In embodiments, the DNA binder comprises TALEs, ZnF, and/or dCas (TABLES 8-17). In embodiments, the helper enzyme comprises a targeting element. In embodiments, the helper enzyme is capable of inserting a donor comprising a transgene in a genomic safe harbor site (GSHS). In embodiments, the binding of a GSHS of a nucleic acid molecule in a mammalian cell is with high target specificity, relative to a control. In embodiments, the control is a composition comprising a helper enzyme comprising an amino acid sequence of SEQ ID NO: 1 or SEQ ID NO: 1 or a nucleic acid comprising one or more of the Exc+Int− mutations and DNA binders in TABLES 1-17 and 19-20 or a codon-optimized forms thereof.

In embodiments, the DNA binding element that directs the transpoase to GSHS is selected from TABLES 3-17 and TABLES 19-20. In embodiments, the guide RNA for targeting human genomic safe harbor sites using any of the gRNA based targeting elements, is selected from GUIDE 44, GUIDE 45-C, GUIDE 46-C, SPG GUIDE1-C, SPG GUIDE2-C, SPG GUIDE3-C, SPG GUIDE4-C, SPG GUIDE5-C, SPG GUIDE6-C, SPG GUIDE7-C, SPG GUIDE8, SPG GUIDE9, SPG GUIDE10, SPG GUIDE11, SPG GUIDE12, SPG GUIDE13, SPG GUIDE14, GUIDE N1, GUIDE N2, GUIDE N3-C, GUIDE 012, GUIDE 013, GUIDE 014-C, AAV GUIDE 12, AAV GUIDE 13c, AAV GUIDE 14, AAV GUIDE 14c, AAV GUIDE 15, AAV GUIDE 16, AAV GUIDE 17, AAV GUIDE 18, AAV GUIDE 19, AAV GUIDE 20, AAV GUIDE 21, AAV GUIDE 22, AAV GUIDE 23, AAV GUIDE 24, AAV GUIDE 25, AAV GUIDE 26, AAV GUIDE 27, AAV GUIDE 28, AAV GUIDE 29, AAV GUIDE 30c, AAV GUIDE 31, AAV GUIDE 32c, AAV GUIDE 33c, AAV GUIDE 34, AAV GUIDE 35, Guide C4-1, Guide C4-2, Guide C4-3, Guide C4-4, Guide C4-5, Guide C4-6, Guide C4-7, Guide C4-8, Guide C4-9, Guide C4-10, Guide C4-11, Guide C4-12, Guide C4-13, Guide C4-14, Guide C4-15, Guide C4-16, Guide C4-17, Guide C4-18, Guide C4-19, Guide C4-20, Guide C4A1, Guide C4A2, Guide C4A3, Guide C4A4, Guide C4A5, Guide C4A6, Guide C4A7, Guide C4A8, Guide C4A9, Guide C4A10, Guide C4A11, Guide C4A12, Guide C4A13, Guide C4A14, Guide C4A15, Guide C4A16, Guide C4A17, Guide C4A18, Guide C4A19, Guide C4A20, Guide C22-1, Guide C22-2, Guide C22-3, Guide C22-4, Guide C22-5, Guide C22-6, Guide C22-7, Guide C22-8, Guide C22-9, Guide C22-10, Guide C22-11, Guide C22-12, Guide C22-13, Guide C22-14, Guide C22-15, Guide C22-16, Guide C22-17, Guide C22-18, Guide C22-19, Guide C22-20, Guide C22A1, Guide C22A2, Guide C22A3, Guide C22A4, Guide C22A5, Guide C22A6, Guide C22A7, Guide C22A8, Guide C22A9, Guide C22A10, Guide C22A11, Guide C22A12, Guide C22A13, Guide C22A14, Guide C22A15, Guide C22A16, Guide C22A17, Guide C22A18, Guide C22A19, Guide C22A20, Guide CX-1, Guide CX-2, Guide CX-3, Guide CX-4, Guide CX-5, Guide CX-6, Guide CX-7, Guide CX-8, Guide CX-9, Guide CX-10, Guide CX-11, Guide CX-12, Guide CX-13, Guide CX-14, Guide CX-15, Guide CX-16, Guide CX-17, Guide CX-18, Guide CX-19, Guide CX-20, TALES: R1, R2, R3, R4, R5, R6, R7, R8, R9, R10, R11, R12, R13, R14, AAV1c, AAV2c, AAV3c, AAV4c, AAV5c, AAV6, AAV7, AAV8, AAV9, AAV10, AAV11, AAV12, AAV13c, AAV14c, AAV15c, AAV16c, AAV17c, TALE4-R001, TALE4-R002, TALE4-F003, TALE4-F004, TALE4-F005, TALE4-F006, TALE4-R007, TALE4-F008, TALE4-R009, TALE4-F010, TALE4-F011, TALE4-F012, TALE4-R013, TALE4-F014, TALE4-F015, TALE4-R016, TALE4-F017, TALE4-F018, TALE4-F019, TALE4-F020, TALE22F-R001, TALE22-F002, TALE22-F003, TALE22-F004, TALE22-F005, TALE22-F006, TALE22-F007, TALE22-F008, TALE22-R009, TALE22-F010, TALE22-F001A, TALE22-F002A, TALE22-R03A, TALE22-F004A, TALE22-F005A, TALE22-F006A, TALE22-R007A, TALE22-R008A, TALE22-R009A, TALE22-F010A, TALE F002, TALE F003, TALE F004, TALE F007, TALE F008, TALE F009, TALE R002, TALE R003, TALE R004, TALE R005, TALE R007, TALE F020, TALE F021, TALE F030, TALE F031, TALE F034, TALE F036, TALE F037, TALE F040, TALE R022, TALE R033, TALE R035, TALE R038, TALE R039, Zinc Fingers: ZnF3a, ZnF5a, ZnF5b, ZnF5c, ZnF5d, ZnF5e, ZnF5f, ZnF5g, ZnF5h, ZnF12a, ZnF13a, ZnF13b, ZnF13c, ZnF11a, ZnF10a, ZnF12b, ZnF13b, ZnF14a, ZnF15a, ZnF16a, ZnF17a, ZnF18a, ZnF19a, ZnF20b, ZnF21b, ZnF22a, ZnF23a, ZnF24a, ZnF31F, ZnF32F, ZnF33F, ZnF34F, ZnF35F, ZnF36F, ZnF37R, ZnF38R, ZnF39R, ZnF1a, ZnF1b, ZnF2a, ZnF3a, ZnF3b, ZnF5aR, ZnF5bR, ZnF6aR, ZnF6bR, ZnF10F, ZnF11F, ZnF12F, ZnF13F, ZnF14R, ZnF15R, ZnF14R, ZnF15R, ZnF41F, ZnF42F, ZnF43F, ZnF44R, ZnF45R, ZnF46R, ZnF47R, ZnF48R.

In embodiments, gRNAs for targeting human genomic safe harbor sites using any of the gRNA-based targeting elements, e.g., without limitation, dCas, in areas of open chromatin are shown in TABLES 3-7.

TABLE 3
Guide RNA sequences targeting the genomic safe harbor site
hROSA26 (hg38 chr3: 9,396,132-9,396,332)

| HROSA26 GUIDE NO. | DNA SEQUENCE |
|---|---|
| GUIDE 44 | AATCGAGAAGCGACTCGACA (SEQ ID NO: 185) |
| GUIDE 45-C | GTCCCTGGGCGTTGCCCTGC (SEQ ID NO: 186) |
| GUIDE 46-C | CCCTGGGCGTTGCCCTGCAG (SEQ ID NO: 187) |
| SPG GUIDE1-C | GAGTGAGCAGCTGTAAGATT (SEQ ID NO: 515) |
| SPG GUIDE2-C | CAGGGGAGTGAGCAGCTGTA (SEQ ID NO: 516) |
| SPG GUIDE3-C | CCTGCAGGGGAGTGAGCAGC (SEQ ID NO: 517) |
| SPG GUIDE 4-C | TGCCCTGCAGGGGAGTGAGC (SEQ ID NO: 518) |
| SPG GUIDE5-C | CGTTGCCCTGCAGGGGAGTG (SEQ ID NO: 519) |
| SPG GUIDE6-C | TGGGCGTTGCCCTGCAGGGG (SEQ ID NO: 520) |
| SPG GUIDE7-C | TTGGTCCCTGGGCGTTGCCC (SEQ ID NO: 521) |
| SPG GUIDE8 | AAGAATCCCGCCCATAATCG (SEQ ID NO: 522) |
| SPG GUIDE9 | AATCCCGCCCATAATCGAGA (SEQ ID NO: 523) |
| SPG GUIDE10 | TCCCGCCCATAATCGAGAAG (SEQ ID NO: 524) |
| SPG GUIDE11 | CCCATAATCGAGAAGCGACT (SEQ ID NO: 525) |
| SPG GUIDE12 | GAGAAGCGACTCGACATGGA (SEQ ID NO: 526) |
| SPG GUIDE13 | GAAGCGACTCGACATGGAGG (SEQ ID NO: 527) |
| SPG GUIDE14 | GCGACTCGACATGGAGGCGA (SEQ ID NO: 528) |
| GUIDE N1 | CCGTGGGAAGATAAACTAAT (SEQ ID NO: 529) |
| GUIDE N2 | TCCCCTGCAGGGCAACGCCC (SEQ ID NO: 530) |
| GUIDE N3-C | GTCGAGTCGCTTCTCGATTA (SEQ ID NO: 531) |
| GUIDE 012 | CGACACCAACTCTAGTCCGT (SEQ ID NO: 532) |
| GUIDE 013 | CAGCTGCTCACTCCCCTGCA (SEQ ID NO: 533) |
| GUIDE 014-C | AGTCGCTTCTCGATTATGGG (SEQ ID NO: 534) |

TABLE 4
Guide RNA sequences targeting the genomic safe harbor site AAVS1
(hg38 chr19: 55,112,850-55,113,324).

| AAVS1 GUIDE NO. | DNA SEQUENCE |
|---|---|
| AAV GUIDE 12 | ACCCTTGGAAGGACCTGGCTGGG (SEQ ID NO: 535) |
| AAV GUIDE 13C | TCCGAGCTTGACCCTTGGAA (SEQ ID NO: 536) |
| AAV GUIDE 14 | GGAGCCACGAAAACAGATCCAGG (SEQ ID NO: 537) |
| AAV GUIDE 14C | TGGTTTCCGAGCTTGACCCT (SEQ ID NO: 538) |
| AAV GUIDE 15 | GGAGCCACGAAAACAGATCCAGG (SEQ ID NO: 539) |
| AAV GUIDE 16 | AGATCCAGGGACACGGTGCTAGG (SEQ ID NO: 540) |
| AAV GUIDE 17 | GACACGGTGCTAGGACAGTGGGG (SEQ ID NO: 541) |
| AAV GUIDE 18 | GAAAATGACCCAACAGCCTCTGG (SEQ ID NO: 542) |
| AAV GUIDE 19 | GCCTGGCCGGCCTGACCACTGGG (SEQ ID NO: 543) |
| AAV GUIDE 20 | CTGAGCACTGAAGGCCTGGCCGG (SEQ ID NO: 544) |
| AAV GUIDE 21 | TGGTTTCCACTGAGCACTGAAGG ( SEQ ID NO: 545) |
| AAV GUIDE 22 | GGTGCTTTCCTGAGGACCGATAG (SEQ ID NO: 546) |
| AAV GUIDE 23 | GCGCTTCCAGTGCTCAGACTAGG (SEQ ID NO: 547) |
| AAV GUIDE 24 | CAGTGCTCAGACTAGGGAAGAGG (SEQ ID NO: 548) |
| AAV GUIDE 25 | GCCCCTCCTCCTTCAGAGCCAGG (SEQ ID NO: 549) |
| AAV GUIDE 26 | TCCTTCAGAGCCAGGAGTCCTGG (SEQ ID NO: 550) |
| AAV GUIDE 27 | CCAAGGGTCAAGCTCGGAAACCA (SEQ ID NO: 551) |
| AAV GUIDE 28 | CTGCAGAGTATCTGCTGGGGTGG (SEQ ID NO: 552) |
| AAV GUIDE 29 | CGTTCCTGCAGAGTATCTGCTGG (SEQ ID NO: 553) |
| AAV GUIDE 30C | GTGGGGAAAATGACCCAACA (SEQ ID NO: 554) |
| AAV GUIDE 31 | GAAGGCCTGGCCGGCCTGAC (SEQ ID NO: 555) |
| AAV GUIDE 32C | ACTCCTGGCTCTGAAGGAGG (SEQ ID NO: 556) |
| AAV GUIDE 33C | GGGCTGGGGGCCAGGACTCC (SEQ ID NO: 557) |
| AAV GUIDE 34 | GTCCTTCCAAGGGTCAAGCT (SEQ ID NO: 558) |
| AAV GUIDE 35 | TCAAGCTCGGAAACCACCCC (SEQ ID NO: 559) |

TABLE 5
Guide RNA sequences targeting a chromosome 4 genomic safe harbor
site (hg38 chr4: 30,793,038-30,793,980)

| CHR4 | GUIDE NO. | DNA SEQUENCE |
|---|---|---|
| GUIDE | C4-1 | ATTGTCTTCACTAAACCCGTTGG (SEQ ID NO: 560) |
| GUIDE | C4-2 | TAAACCCGTTGGGAATACAATGG (SEQ ID NO: 561) |
| GUIDE | C4-3 | TTGTCTTCACTAAACCCGTTGGG (SEQ ID NO: 562) |
| GUIDE | C4-4 | TGATTCATAGGAGTCTATTAAGG (SEQ ID NO: 563) |
| GUIDE | C4-5 | TTACATATGCTTCGAGTTTGTGG (SEQ ID NO: 564) |
| GUIDE | C4-6 | ACTCTTAAGGTAGGACTAATTGG (SEQ ID NO: 565) |
| GUIDE | C4-7 | TATGTGTGCAATAGCGTTAAAGG (SEQ ID NO: 566) |
| GUIDE | C4-8 | CGTTGGGAATACAATGGCTTAGG (SEQ ID NO: 567) |
| GUIDE | C4-9 | TCACAATGGAACTCTGCCTTTGG (SEQ ID NO: 568) |
| GUIDE | C4-10 | GACCACAAATCAATGCCCAAAGG (SEQ ID NO: 569) |
| GUIDE | C4-11 | CTAAGCCATTGTATTCCCAACGG (SEQ ID NO: 570) |
| GUIDE | C4-12 | AGCATTCTGGAGTGTCACAATGG (SEQ ID NO: 571) |
| GUIDE | C4-13 | CAATAGCCCACTTTAATACTAGG (SEQ ID NO: 572) |
| GUIDE | C4-14 | CTTTATCCAAGTGAATCCTTTGG (SEQ ID NO: 573) |
| GUIDE | C4-15 | GGCATTGATTTGTGGTCATTTGG (SEQ ID NO: 574) |
| GUIDE | C4-16 | TAAGCCATTGTATTCCCAACGGG (SEQ ID NO: 575) |
| GUIDE | C4-17 | AATACAATCACTCTTAAGGTAGG (SEQ ID NO: 576 |
| GUIDE | C4-18 | GAAGTACCTTTCACTATTTTGGG (SEQ ID NO: 577) |
| GUIDE | C4-19 | CAAGCAACAAATGACTTCTAAGG (SEQ ID NO: 578) |
| GUIDE | C4-20 | TTTGAATACAATCACTCTTAAGG (SEQ ID NO: 579) |
| GUIDE | C4A1 | ACAAACGGACTACGTAAACTTGG (SEQ ID NO: 580) |
| GUIDE | C4A2 | ACAAGATGTGAACACGACGATGG (SEQ ID NO: 581) |
| GUIDE | C4A3 | GTTGCACCGTTGATTCCTTCAGG (SEQ ID NO: 582) |
| GUIDE | C4A4 | AGTAATATTGAATTAGGGCGTGG (SEQ ID NO: 583) |
| GUIDE | C4A5 | CCTGATGTTGGCTCGACATTAGG (SEQ ID NO: 584) |
| GUIDE | C4A6 | CTTTGTTGGGTCTTAGCTTAAGG (SEQ ID NO: 585) |
| GUIDE | C4A7 | TCGGAACAGCTCCTTCCTGAAGG (SEQ ID NO: 586) |
| GUIDE | C4A8 | AGTAGTTTCTGAGGTCATGTTGG (SEQ ID NO: 587) |
| GUIDE | C4A9 | CTTGAAAATACGATGATGTGAGG (SEQ ID NO: 588) |
| GUIDE | C4A10 | GCATTAATCTAGAGAGAGGGAGG (SEQ ID NO: 589) |
| GUIDE | C4A11 | GGGTCATGTTAGAATTCATGTGG (SEQ ID NO: 590) |
| GUIDE | C4A12 | TGATGCATTAATCTAGAGAGAGG (SEQ ID NO: 591) |
| GUIDE | C4A13 | ACATCATCGTATTTTCAAGTTGG (SEQ ID NO: 592) |
| GUIDE | C4A14 | CTAGCTGACAAACATGTGAGTGG (SEQ ID NO: 593) |
| GUIDE | C4A15 | AACATGACCCAAGTGAGTCCAGG (SEQ ID NO: 594) |
| GUIDE | C4A16 | GATTCCGTATTTGCTTTGTTGGG (SEQ ID NO: 595) |
| GUIDE | C4A17 | TACGATGATGTGAGGAAATAAGG (SEQ ID NO: 596) |
| GUIDE | C4A18 | GTAATATGTCTAAGTACTGATGG (SEQ ID NO: 597) |
| GUIDE | C4A19 | GTAAAGTGAGCTGGTTCATTAGG (SEQ ID NO: 598) |
| GUIDE | C4A20 | ACTAGAGTCCTTAAGAAGGGGGG (SEQ ID NO: 599) |

CHOPCHOP algorithm

TABLE 6
Guide RNA sequences targeting a chromosome 22 genomic safe harbor
site (hg38 chr22: 35,373,428-35,380,000).

| CHR22 | GUIDE NO. | DNA SEQUENCE |
|---|---|---|
| GUIDE | C22-1 | ATAACACGTGAGCCGTCCTAAGG (SEQ ID NO: 600) |
| GUIDE | C22-2 | GGAAGACTTTTCTCTATACGAGG (SEQ ID NO: 601) |
| GUIDE | C22-3 | GCATTCCTTTCATCCATGGCAGG (SEQ ID NO: 602) |
| GUIDE | C22-4 | GACATATGGTTATAAAAATCAGG (SEQ ID NO: 603) |
| GUIDE | C22-5 | GGAGTGCAGTCCCTGACATATGG (SEQ ID NO: 604) |
| GUIDE | C22-6 | GTGGGTTAGGGTGGTTAACTGGG (SEQ ID NO: 605) |
| GUIDE | C22-7 | AGGTGCAAAAAGGTTGCTGTGGG (SEQ ID NO: 606) |
| GUIDE | C22-8 | CGTGACAAGGCAAAGTGGCGTGG (SEQ ID NO: 607) |
| GUIDE | C22-9 | GAAGGACTGCCCCTGACGTCAGG (SEQ ID NO: 608) |
| GUIDE | C22-10 | CTGCCCCTGACGTCAGGAGTTGG (SEQ ID NO: 609) |
| GUIDE | C22-11 | TGTGGGTTAGGGTGGTTAACTGG (SEQ ID NO: 610) |
| GUIDE | C22-12 | ACCCTTTTAGAGTTTTCTGCTGG (SEQ ID NO: 611) |
| GUIDE | C22-13 | AACTTCCTGCCATGGATGAAAGG (SEQ ID NO: 612) |
| GUIDE | C22-14 | GCAAAAAGGTTGCTGTGGGTTGG (SEQ ID NO: 613) |
| GUIDE | C22-15 | AATTTGGGGGTAGATAGGCATGG (SEQ ID NO: 614) |
| GUIDE | C22-16 | AGAAAACTCTAAAAGGGTATAGG (SEQ ID NO: 615) |
| GUIDE | C22-17 | ATTAGCATTCCTTTCATCCATGG (SEQ ID NO: 616) |
| GUIDE | C22-18 | CCCAGCAGAAAACTCTAAAAGGG (SEQ ID NO: 617) |
| GUIDE | C22-19 | CAGGTGCAAAAAGGTTGCTGTGG (SEQ ID NO: 618) |
| GUIDE | C22-20 | GCAAGAGATGAAATTCCATATGG (SEQ ID NO: 619) |
| GUIDE | C22A1 | GGGCTGTTCTAACGAAGTCTGGG (SEQ ID NO: 620) |
| GUIDE | C22A2 | TGTCCATTCAGCGACCCTAGAGG (SEQ ID NO: 621) |
| GUIDE | C22A3 | GGCTGTTCTAACGAAGTCTGGGG (SEQ ID NO: 622) |
| GUIDE | C22A4 | GTCCATTCAGCGACCCTAGAGGG (SEQ ID NO: 623) |
| GUIDE | C22A5 | GGGGCTGTTCTAACGAAGTCTGG (SEQ ID NO: 624) |
| GUIDE | C22A6 | GGCTGAATCAGCATGCGAAAGGG (SEQ ID NO: 625) |
| GUIDE | C22A7 | TTCCAATGGGGGGCATAGCCTGG (SEQ ID NO: 626) |
| GUIDE | C22A8 | TACCCTCTAGGGTCGCTGAATGG (SEQ ID NO: 627) |
| GUIDE | C22A9 | ATCCTCTTGGGCCTTATAAGAGG (SEQ ID NO: 628) |
| GUIDE | C22A10 | GGCCAGGCTATGCCCCCCATTGG (SEQ ID NO: 629) |
| GUIDE | C22A11 | CTAGAGGACCAGAACAACTCTGG (SEQ ID NO: 630) |
| GUIDE | C22A12 | TCCCTCTTATAAGGCCCAAGAGG (SEQ ID NO: 631) |
| GUIDE | C22A13 | AGGCTGAATCAGCATGCGAAAGG (SEQ ID NO: 632) |
| GUIDE | C22A14 | GGACCAGAACAACTCTGGCCTGG (SEQ ID NO: 633) |
| GUIDE | C22A15 | GGGCTTTTATTTGGCCCAGCAGG (SEQ ID NO: 634) |
| GUIDE | C22A16 | GTCGCTGAATGGACAGACTCTGG (SEQ ID NO: 635) |
| GUIDE | C22A17 | CTCATGAGTTTTACCCTCTAGGG (SEQ ID NO: 636) |
| GUIDE | C22A18 | TCCTCTTGGGCCTTATAAGAGGG (SEQ ID NO: 637) |
| GUIDE | C22A19 | TCTTGGGCCTTATAAGAGGGAGG (SEQ ID NO: 638) |
| GUIDE | C22A20 | TAGAACAGCCCCCCACACAGTGG (SEQ ID NO: 639) |

TABLE 7
Guide RNA sequences targeting chromosome X (HPRT)
(hg38 chrX: 134,475,808-134,476,794).

| CHRX GUIDE NO. | DNA SEQUENCE |
|---|---|
| GUIDE CX-1 | GTTACGTTATGACTAATCTTTGG (SEQ ID NO: 640) |
| GUIDE CX-2 | TACGTTATGACTAATCTTTGGGG (SEQ ID NO: 641) |
| GUIDE CX-3 | GGAAGTAGTGTTATGATGTATGG (SEQ ID NO: 642) |
| GUIDE CX-4 | GTTATGATGTATGGGCATAAAGG (SEQ ID NO: 643) |
| GUIDE CX-5 | GAAGTAGTGTTATGATGTATGGG (SEQ ID NO: 644) |
| GUIDE CX-6 | ATAGCTGCTGGCAGTATAACTGG (SEQ ID NO: 645) |
| GUIDE CX-7 | GCATCACAACATTGACACTGTGG (SEQ ID NO: 646) |
| GUIDE CX-8 | AAGGCGAGTTTCTACAAAGATGG (SEQ ID NO: 647) |
| GUIDE CX-9 | TTACGTTATGACTAATCTTTGGG (SEQ ID NO: 648) |
| GUIDE CX-10 | CAAGACTGATTAAGACTGATGGG (SEQ ID NO: 649) |
| GUIDE CX-11 | AGCAGCAATGTATTAAAGGCTGG (SEQ ID NO: 650) |
| GUIDE CX-12 | CTACAGGATTGATGTAAACATGG (SEQ ID NO: 651) |
| GUIDE CX-13 | TGGGCATAAAGGGTTTTAATGGG (SEQ ID NO: 652) |
| GUIDE CX-14 | ACATCAATCCTGTAGGTGATTGG (SEQ ID NO: 653) |
| GUIDE CX-15 | ATTCTAGTCATTATAGCTGCTGG (SEQ ID NO: 654) |
| GUIDE CX-16 | CATCAATCCTGTAGGTGATTGGG (SEQ ID NO: 654) |
| GUIDE CX-17 | GTTATAAGATCAATTCTGAGTGG (SEQ ID NO: 656) |
| GUIDE CX-18 | GGCAGACTGTGGATCAAAAGTGG (SEQ ID NO: 657) |
| GUIDE CX-19 | ATGGCTGCCCAATCACCTACAGG (SEQ ID NO: 658) |
| GUIDE CX-20 | TCAAAGCATGTACTTAGAGTTGG (SEQ ID NO: 659) |

In embodiments, the gRNA comprises one or more of the sequences outlined herein or a variant sequence having at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

In embodiments, a Cas-based targeting element comprises Cas12 or a variant thereof, e.g., without limitation, Cas12a (e.g., dCas12a), or Cas12j (e.g., dCas12j), or Cas12k (e.g., dCas12k). In embodiments, the targeting element comprises a Cas12 enzyme guide RNA complex. In embodiments, comprises a nuclease-deficient dCas12 guide RNA complex, optionally dCas12j guide RNA complex or dCas12a guide RNA complex.

In embodiments, the targeting element is selected from a zinc finger (ZF), transcription activator-like effector (TALE), meganuclease, and clustered regularly interspaced short palindromic repeat (CRISPR)-associated protein, any of which are, in embodiments, catalytically inactive. In embodiments, the CRISPR-associated protein is selected from Cas9, CasX, CasY, Cas12a (Cpf1), and gRNA complexes thereof. In embodiments, the CRISPR-associated protein is selected from Cas9, xCas9, Cas 6, Cas7, Cas8, Cas12a (Cpf1), Cas13a, Cas14, CasX, CasY, a Class 1 Cas protein, a Class 2 Cas protein, MAD7, MG1 nuclease, MG2 nuclease, MG3 nuclease, or catalytically inactive forms thereof, and gRNA complexes thereof.

In embodiments, the helper enzyme of the present disclosure is capable of inserting a donor DNA at a TA dinucleotide site, or a TTAA tetranucleotide site, or a ttTTAAaa octanucleotide site, or a taTTAAta octanucleotide site, in a genomic safe harbor site (GSHS) of a nucleic acid molecule. The helper enzyme of the present disclosure is suitable for causing insertion of the donor DNA in a GSHS when contacted with a biological cell.

In embodiments, the targeting element is suitable for directing the helper enzyme of the present disclosure to the GSHS sequence.

In embodiments, the targeting element comprises transcription activator-like effector (TALE) DNA binding domain (DBD). The TALE DBD comprises one or more repeat sequences. For example, in embodiments, the TALE DBD comprises about 14, or about 15, or about, 16, or about 17, or about 18, or about 18.5 repeat sequences. In embodiments, the TALE DBD repeat sequences comprise 33 or 34 amino acids.

In embodiments, the one or more of the TALE DBD repeat sequences comprise a repeat variable di-residue (RVD) at residue 12 or 13 of the 33 or 34 amino acids.

In embodiments, the targeting element (e.g., TALE or Cas (e.g., Cas9 or Cas12, or variants thereof) DBDs cause the the helper enzyme of the present disclosure to bind specifically to human GSHS. In embodiments, the TALEs or Cas DBDs sequester the helper to GSHS and promote transposition to nearby TA dinucleotide, or TTAA tetranucleotide, or ttTTAAaa octanucleotide, or taTTAAta octanucleotide sites which can be located in proximity to the repeat variable di-residues (RVD) TALE or gRNA nucleotide sequences. The GSHS regions are located in open chromatin sites that are susceptible to helper activity. Accordingly, the helper enzyme of the present disclosure does not only operate based on its ability to recognize TA or TTAA sites, but it also directs a donor DNA (having a transgene) to specific locations in proximity to a TALE or Cas DBD. The helper enzyme of the present disclosure in accordance with embodiments of the present disclosure has negligible risk of genotoxicity and exhibits superior features as compared to existing gene therapies.

In embodiments, the helper enzyme of the present disclosure is mutated to be characterized by reduced or inhibited binding of off-target sequences and consequently reliant on a DBD fused thereto, such as a TALE or Cas DBD, for transposition.

The described cells, compositions, and methods allow reducing vector and transgene insertions that increase a mutagenic risk. The described cells and methods make use of a gene transfer system that reduces genotoxicity compared to viral- and nuclease-mediated gene therapies.

In embodiments, TALE or Cas DBDs are customizable, such as a TALE or Cas DBDs is selected for targeting a specific genomic location. In embodiments, the genomic location is in proximity to a TA dinucleotide site, or a TTAA (SEQ ID NO: 440) tetranucleotide site, or a ttTTAAaa octanucleotide site, or a taTTAAta octanucleotide site.

Embodiments of the present disclosure make use of the ability of TALE or Cas or dCas9/gRNA DBDs to target specific sites in a host genome. The DNA targeting ability of a TALE or Cas DBD or dCas9/gRNA DBD is provided by TALE repeat sequences (e.g., modular arrays) or gRNA which are linked together to recognize flanking DNA sequences. Each TALE or gRNA can recognize certain base pair(s) or residue(s).

TALE nucleases (TALENs) are a known tool for genome editing and introducing targeted double-stranded breaks. TALENs comprise endonucleases, such as FokI nuclease domain, fused to a customizable DBD. This DBD is composed of highly conserved repeats from TALEs, which are proteins secreted by Xanthomonas bacteria to alter transcription of genes in host plant cells. The DBD includes a repeated highly conserved 33-34 amino acid sequence with divergent 12th and 13th amino acids. These two positions, referred to as the RVD, are highly variable and show a strong correlation with specific base pair or nucleotide recognition. This straightforward relationship between amino acid sequence and DNA recognition has allowed for the engineering of specific DBDs by selecting a combination of repeat segments containing the appropriate RVDs. Boch et al. Nature Biotechnology. 2011; 29 (2): 135-6.

Accordingly, TALENs can be readily designed using a “protein-DNA code” that relates modular DNA-binding TALE repeat domains to individual bases in a target-binding site. See Joung et al. Nat Rev Mol Cell Biol. 2013; 14(1):49-55. doi:10.1038/nrm3486. The following table, for example, shows such code:

TABLE 18

| RVD | Nucleotide | RVD | Nucleotide |
|---|---|---|---|
| HD | C | NI | A |
| NH | G | NN | G, A |
| NK | G | NS | G, C, A |
| NG | T, mC | | |

It has been demonstrated that TALENs can be used to target essentially any DNA sequence of interest in human cell. Miller et al. Nat Biotechnol. 2011; 29:143-148. Guidelines for selection of potential target sites and for use of particular TALE repeat domains (harboring NH residues at the hypervariable positions) for recognition of G bases have been proposed. See Streubel et al. Nat Biotechnol. 2012; 30:593-595.

Accordingly, in embodiments, the TALE DBD comprises one or more repeat sequences. In embodiments, the TALE DBD comprises about 5, or about 6, or about 7, or about 8, or about 9, or about 10, or about 11, or about 12, or about 13, or about 14, or about 15, or about, 16, or about 17, or about 18, or about 18.5 repeat sequences. In embodiments, the TALE DBD comprises about 8.5 repeat sequences. In embodiments, the TALE DBD repeat sequences comprise 33 or 34 amino acids.

In embodiments, the one or more of the TALE DBD repeat sequences comprise an RVD at residue 12 or 13 of the 33 or 34 amino acids. The RVD can recognize certain base pair(s) or residue(s). In embodiments, the RVD recognizes one base pair in the nucleic acid molecule. In embodiments, the RVD recognizes a C residue in the nucleic acid molecule and is selected from HD, N (gap), HA, ND, and HI. In embodiments, the RVD recognizes a G residue in the nucleic acid molecule and is selected from NN, NH, NK, HN, and NA. In embodiments, the RVD recognizes an A residue in the nucleic acid molecule and is selected from NI and NS. In embodiments, the RVD recognizes a T residue in the nucleic acid molecule and is selected from NG, HG, H (gap), and IG.

In embodiments, the GSHS is in an open chromatin location in a chromosome. In embodiments, the GSHS is selected from adeno-associated virus site 1 (AAVS1), chemokine (C—C motif) receptor 5 (CCR5) gene, HIV-1 coreceptor; and human Rosa26 locus. In embodiments, the GSHS is located on human chromosome 2, 3, 4, 6, 10, 11, 17, 19, or 22.

In embodiments, the GSHS is selected from TALC1, TALC2, TALC3, TALC4, TALC5, TALC7, TALC8, AVS1, AVS2, AVS3, ROSA1, ROSA2, TALER1, TALER2, TALER3, TALER4, TALER5, SHCHR2-1, SHCHR2-2, SHCHR2-3, SHCHR2-4, SHCHR4-1, SHCHR4-2, SHCHR4-3, SHCHR6-1, SHCHR6-2, SHCHR6-3, SHCHR6-4, SHCHR10-1, SHCHR10-2, SHCHR10-3, SHCHR10-4, SHCHR10-5, SHCHR11-1, SHCHR11-2, SHCHR11-3, SHCHR17-1, SHCHR17-2, SHCHR17-3, and SHCHR17-4.

In embodiments, the GSHS comprises one or more of TGGCCGGCCTGACCACTGG (SEQ ID NO: 23), TGAAGGCCTGGCCGGCCTG (SEQ ID NO: 24), TGAGCACTGAAGGCCTGGC (SEQ ID NO: 25), TCCACTGAGCACTGAAGGC (SEQ ID NO: 26), TGGTTTCCACTGAGCACTG (SEQ ID NO: 27), TGGGGAAAATGACCCAACA (SEQ ID NO: 28), TAGGACAGTGGGGAAAATG (SEQ ID NO: 29), TCCAGGGACACGGTGCTAG (SEQ ID NO: 30), TCAGAGCCAGGAGTCCTGG (SEQ ID NO: 31), TCCTTCAGAGCCAGGAGTC (SEQ ID NO: 32), TCCTCCTTCAGAGCCAGGA (SEQ ID NO: 33), TCCAGCCCCTCCTCCTTCA (SEQ ID NO: 34), TCCGAGCTTGACCCTTGGA (SEQ ID NO: 35), TGGTTTCCGAGCTTGACCC (SEQ ID NO: 36), TGGGGTGGTTTCCGAGCTT (SEQ ID NO: 37), TCTGCTGGGGTGGTTTCCG (SEQ ID NO: 38), TGCAGAGTATCTGCTGGGG (SEQ ID NO: 39), CCAATCCCCTCAGT (SEQ ID NO: 40), CAGTGCTCAGTGGAA (SEQ ID NO: 41), GAAACATCCGGCGACTCA (SEQ ID NO: 42), TCGCCCCTCAAATCTTACA (SEQ ID NO: 43), TCAAATCTTACAGCTGCTC (SEQ ID NO: 44), TCTTACAGCTGCTCACTCC (SEQ ID NO: 45), TACAGCTGCTCACTCCCCT (SEQ ID NO: 46), TGCTCACTCCCCTGCAGGG (SEQ ID NO: 47), TCCCCTGCAGGGCAACGCC (SEQ ID NO: 48), TGCAGGGCAACGCCCAGGG (SEQ ID NO: 49) TCTCGATTATGGGGGGGAT (SEQ ID NO: 50), TCGCTTCTCGATTATGGGC (SEQ ID NO: 51), TGTCGAGTCGCTTCTCGAT (SEQ ID NO: 52), TCCATGTCGAGTCGCTTCT (SEQ ID NO: 53), TCGCCTCCATGTCGAGTCG (SEQ ID NO: 54) TCGTCATCGCCTCCATGTC (SEQ ID NO: 55), TGATCTCGTCATCGCCTCC (SEQ ID NO: 56), GCTTCAGCTTCCTA (SEQ ID NO: 57), CTGTGATCATGCCA (SEQ ID NO: 58), ACAGTGGTACACACCT (SEQ ID NO: 59), CCACCCCCCACTAAG (SEQ ID NO: 60), CATTGGCCGGGCAC (SEQ ID NO: 61), GCTTGAACCCAGGAGA (SEQ ID NO: 62), ACACCCGATCCACTGGG (SEQ ID NO: 63), GCTGCATCAACCCC (SEQ ID NO: 64), GCCACAAACAGAAATA (SEQ ID NO: 65), GGTGGCTCATGCCTG (SEQ ID NO: 66), GATTTGCACAGCTCAT (SEQ ID NO: 67), AAGCTCTGAGGAGCA (SEQ ID NO: 68), CCCTAGCTGTCCC (SEQ ID NO: 69), GCCTAGCATGCTAG (SEQ ID NO: 70), ATGGGCTTCACGGAT (SEQ ID NO: 71), GAAACTATGCCTGC (SEQ ID NO: 72), GCACCATTGCTCCC (SEQ ID NO: 73), GACATGCAACTCAG (SEQ ID NO: 74), ACACCACTAGGGGT (SEQ ID NO: 75), GTCTGCTAGACAGG (SEQ ID NO: 76), GGCCTAGACAGGCTG (SEQ ID NO: 77), GAGGCATTCTTATCG (SEQ ID NO: 78), GCCTGGAAACGTTCC (SEQ ID NO: 79), GTGCTCTGACAATA (SEQ ID NO: 80), GTTTTGCAGCCTCC (SEQ ID NO: 81), ACAGCTGTGGAACGT (SEQ ID NO: 82), GGCTCTCTTCCTCCT (SEQ ID NO: 83), CTATCCCAAAACTCT (SEQ ID NO: 84), GAAAAACTATGTAT (SEQ ID NO: 85), AGGCAGGCTGGTTGA (SEQ ID NO: 86), CAATACAACCACGC (SEQ ID NO: 87), ATGACGGACTCAACT (SEQ ID NO: 88), CACAACATTTGTAA (SEQ ID NO: 89), and ATTTCCAGTGCACA (SEQ ID NO: 90).

In embodiments, the TALE DBD binds to one of TGGCCGGCCTGACCACTGG (SEQ ID NO: 23), TGAAGGCCTGGCCGGCCTG (SEQ ID NO: 24), TGAGCACTGAAGGCCTGGC (SEQ ID NO: 25), TCCACTGAGCACTGAAGGC (SEQ ID NO: 26), TGGTTTCCACTGAGCACTG (SEQ ID NO: 27), TGGGGAAAATGACCCAACA (SEQ ID NO: 28), TAGGACAGTGGGGAAAATG (SEQ ID NO: 29), TCCAGGGACACGGTGCTAG (SEQ ID NO: 30), TCAGAGCCAGGAGTCCTGG (SEQ ID NO: 31), TCCTTCAGAGCCAGGAGTC (SEQ ID NO: 32), TCCTCCTTCAGAGCCAGGA (SEQ ID NO: 33), TCCAGCCCCTCCTCCTTCA (SEQ ID NO: 34), TCCGAGCTTGACCCTTGGA (SEQ ID NO: 35), TGGTTTCCGAGCTTGACCC (SEQ ID NO: 36), TGGGGTGGTTTCCGAGCTT (SEQ ID NO: 37), TCTGCTGGGGTGGTTTCCG (SEQ ID NO: 38), TGCAGAGTATCTGCTGGGG (SEQ ID NO: 39), CCAATCCCCTCAGT (SEQ ID NO: 40), CAGTGCTCAGTGGAA (SEQ ID NO: 41), GAAACATCCGGCGACTCA (SEQ ID NO: 42), TCGCCCCTCAAATCTTACA (SEQ ID NO: 43), TCAAATCTTACAGCTGCTC (SEQ ID NO: 44), TCTTACAGCTGCTCACTCC (SEQ ID NO: 45), TACAGCTGCTCACTCCCCT (SEQ ID NO: 46), TGCTCACTCCCCTGCAGGG (SEQ ID NO: 47), TCCCCTGCAGGGCAACGCC (SEQ ID NO: 48), TGCAGGGCAACGCCCAGGG (SEQ ID NO: 49), TCTCGATTATGGGGGGGAT (SEQ ID NO: 50), TCGCTTCTCGATTATGGGC (SEQ ID NO: 51), TGTCGAGTCGCTTCTCGAT (SEQ ID NO: 52), TCCATGTCGAGTCGCTTCT (SEQ ID NO: 53), TCGCCTCCATGTCGAGTCG (SEQ ID NO: 54), TCGTCATCGCCTCCATGTC (SEQ ID NO: 55), TGATCTCGTCATCGCCTCC (SEQ ID NO: 56), GCTTCAGCTTCCTA (SEQ ID NO: 57), CTGTGATCATGCCA (SEQ ID NO: 58), ACAGTGGTACACACCT (SEQ ID NO: 59), CCACCCCCCACTAAG (SEQ ID NO: 60), CATTGGCCGGGCAC (SEQ ID NO: 61), GCTTGAACCCAGGAGA (SEQ ID NO: 62), ACACCCGATCCACTGGG (SEQ ID NO: 63), GCTGCATCAACCCC (SEQ ID NO: 64), GCCACAAACAGAAATA (SEQ ID NO: 65), GGTGGCTCATGCCTG (SEQ ID NO: 66), GATTTGCACAGCTCAT (SEQ ID NO: 67), AAGCTCTGAGGAGCA (SEQ ID NO: 68), CCCTAGCTGTCCC (SEQ ID NO: 69), GCCTAGCATGCTAG (SEQ ID NO: 70), ATGGGCTTCACGGAT (SEQ ID NO: 71), GAAACTATGCCTGC (SEQ ID NO: 72), GCACCATTGCTCCC (SEQ ID NO: 73), GACATGCAACTCAG (SEQ ID NO: 74), ACACCACTAGGGGT (SEQ ID NO: 75), GTCTGCTAGACAGG (SEQ ID NO: 76), GGCCTAGACAGGCTG (SEQ ID NO: 77), GAGGCATTCTTATCG (SEQ ID NO: 78), GCCTGGAAACGTTCC (SEQ ID NO: 79), GTGCTCTGACAATA (SEQ ID NO: 80), GTTTTGCAGCCTCC (SEQ ID NO: 81), ACAGCTGTGGAACGT (SEQ ID NO: 82), GGCTCTCTTCCTCCT (SEQ ID NO: 83), CTATCCCAAAACTCT (SEQ ID NO: 84), GAAAAACTATGTAT (SEQ ID NO: 85), AGGCAGGCTGGTTGA (SEQ ID NO: 86), CAATACAACCACGC (SEQ ID NO: 87), ATGACGGACTCAACT (SEQ ID NO: 88), CACAACATTTGTAA (SEQ ID NO: 89), and ATTTCCAGTGCACA (SEQ ID NO: 90).

In embodiments, the TALE DBD comprises one or more of

NH NH HD HD NH NH HD HD NG NH NI HD HD NI HD NG NH
NH,
NH NI NI NH NH HD HD NG NH NH HD HD NH NH HD HD NG
NH,
NH NI NH HD NI HD NG NH NI NI NH NH HD HD NG NH NH
HD,
HD HD NI HD NG NH NI NH HD NI HD NG NH NI NI NH NH
HD,
NH NH NG NG NG HD HD NI HD NG NH NI NH HD NI HD NG
NH,
NH NH NH NH NI NI NI NI NG NH NI HD HD HD NI NI HD
NI,
NI NH NH NI HD NI NH NG NH NH NH NH NI NI NI NI NG
NH,
HD HD NI NH NH NH NI HD NI HD NH NH NG NH HD NG NI
NH,
HD NI NH NI NH HD HD NI NH NH NI NH NG HD HD NG NH
NH,
HD HD NG NG HD NI NH NI NH HD HD NI NH NH NI NH NG
HD,
HD HD NG HD HD NG NG HD NI NH NI NH HD HD NI NH NH
NI,
HD HD NI NH HD HD HD HD NG HD HD NG HD HD NG NG HD
NI,
HD HD NH NI NH HD NG NG NH NI HD HD HD NG NG NH NH
NI,
NH NH NG NG NG HD HD NH NI NH HD NG NG NH NI HD HD
HD,
NH NH NH NH NG NH NH NG NG NG HD HD NH NI NH HD NG
NG,
HD NG NH HD NG NH NH NH NH NG NH NH NG NG NG HD HD
NH,
NH HD NI NH NI NH NG NI NG HD NG NH HD NG NH NH NH
NH,
HD HD NI NI NG HD HD HD HD NG HD NI NH NG,
HD NI NH NG NH HD NG HD NI NH NG NH NH NI NI,
NH NI NI NI HD NI NG HD HD NH NH HD NH NI HD NG HD
NI,
HD NH HD HD HD HD NG HD NI NI NI NG HD NG NG NI HD
NI,
HD NI NI NI NG HD NG NG NI HD NI NH HD NG NH HD NG
HD,
HD NG NG NI HD NI NH HD NG NH HD NG HD NI HD NG HD
HD,
NI HD NI NH HD NG NH HD NG HD NI HD NG HD HD HD HD
NG,
NH HD NG HD NI HD NG HD HD HD HD NG NH HD NI NH NH
NH,
HD HD HD HD NG NH HD NI NH NH NH HD NI NI HD NH HD
HD,
NH HD NI NH NH NH HD NI NI HD NH HD HD HD NI NH NH
NH,
HD NG HD NH NI NG NG NI NG NH NH NH HD NH NH NH NI
NG,
HD NH HD NG NG HD NG HD NH NI NG NG NI NG NH NH NH
HD,
NH NG HD NH NI NH NG HD NH HD NG NG HD NG HD NH NI
NG,
HD HD NI NG NH NG HD NH NI NH NG HD NH HD NG NG HD
NG,
HD NH HD HD NG HD HD NI NG NH NG HD NH NI NH NG HD
NH,
HD NH NG HD NI NG HD NH HD HD NG HD HD NI NG NH NG
HD,
NH NI NG HD NG HD NH NG HD NI NG HD NH HD HD NG HD
HD,
NH HD NG NG HD NI NH HD NG NG HD HD NG NI,
HD NG NK NG NH NI NG HD NI NG NH HD HD NI,
NI HD NI NN NG NN NN NG NI HD NI HD NI HD HD NG,
HD HD NI HD HD HD HD HD HD NI HD NG NI NI NN,
HD NI NG NG NN NN HD HD NN NN NN HD NI HD,
NN HD NG NG NN NI NI HD HD HD NI NN NN NI NN NI,
NI HD NI HD HD HD NN NI NG HD HD NI HD NG NN NN NN,
NN HD NG NN HD NI NG HD NI NI HD HD HD HD,
NN NN HD NI HD NN NI NI NI HD NI HD HD HD NG HD HD,
NN NN NG NN NN HD NG HD NI NG NN HD HD NG NN,
NN NI NG NG NG NN HD NI HD NI NN HD NG HD NI NG,
NI NI NH HD NG HD NG NH NI NH NH NI NH HD,
HD HD HD NG NI NK HD NG NH NG HD HD HD HD,
NH HD HD NG NI NH HD NI NG NH HD NG NI NH,
NI NG NH NH NH HD NG NG HD NI HD NH NH NI NG,
NH NI NI NI HD NG NI NG NH HD HD NG NH HD,
NH HD NI HD HD NI NG NG NH HD NG HD HD HD,
NH NI HD NI NG NH HD NI NI HD NG HD NI NH,
NI HD NI HD HD NI HD NG NI NH NH NH NH NG,
NH NG HD NG NH HD NG NI NH NI HD NI NH NH,
NH NH HD HD NG NI NH NI HD NI NH NH HD NG NH,
NH NI NH NH HD NI NG NG HD NG NG NI NG HD NH,
NN HD HD NG NN NN NI NI NI HD NN NG NG HD HD,
NN NG NN HD NG HD NG NN NI HD NI NI NG NI,
NN NG NG NG NG NN HD NI NN HD HD NG HD HD,
NI HD NI NN HD NG NN NG NN NN NI NI HD NN NG,
HD NI NI NN NI HD HD NN NI NN HD NI HD NG NN HD NG
NN,
HD NG NI NG HD HD HD NI NI NI NI HD NG HD NG,
NH NI NI NI NI NI HD NG NI NG NH NG NI NG,
NI NH NH HD NI NH NH HD NG NH NH NG NG NH NI,
HD NI NI NG NI HD NI NI HD HD NI HD NN HD,
NI NG NN NI HD NN NN NI HD NG HD NI NI HD NG,
HD NI HD NI NI HD NI NG NG NG NN NG NI NI,
and
NI NG NG NG HD HD NI NN NG NN HD NI HD NI.

In embodiments, the TALE DBD comprises one or more of the sequences outlined herein or a variant sequence having at least about 90%, or at least about 93%, or at least about 95%, or at least about 97%, or at least about 98%, or at least about 99% identity thereto, or at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

In embodiments, the GSHS and the TALE DBD sequences are selected from:

(SEQ ID NO: 23)
TGGCCGGCCTGACCACTGG
and
NH NH HD HD NH NH HD HD NG NH NI HD HD NI HD NG NH
NH;
(SEQ ID NO: 24)
TGAAGGCCTGGCCGGCCTG
and
NH NI NI NH NH HD HD NG NH NH HD HD NH NH HD HD NG
NH;
(SEQ ID NO: 25)
TGAGCACTGAAGGCCTGGC
and
NH NI NH HD NI HD NG NH NI NI NH NH HD HD NG NH NH
HD;
(SEQ ID NO: 26)
TCCACTGAGCACTGAAGGC
and
HD HD NI HD NG NH NI NH HD NI HD NG NH NI NI NH NH
HD;
(SEQ ID NO: 27)
TGGTTTCCACTGAGCACTG
and
NH NH NG NG NG HD HD NI HD NG NH NI NH HD NI HD NG
NH;
(SEQ ID NO: 28)
TGGGGAAAATGACCCAACA
and
NH NH NH NH NI NI NI NI NG NH NI HD HD HD NI NI HD
NI;
(SEQ ID NO: 29)
TAGGACAGTGGGGAAAATG
and
NI NH NH NI HD NI NH NG NH NH NH NH NI NI NI NI NG
NH;
(SEQ ID NO: 30)
TCCAGGGACACGGTGCTAG
and
HD HD NI NH NH NH NI HD NI HD NH NH NG NH HD NG NI
NH;
(SEQ ID NO: 31)
TCAGAGCCAGGAGTCCTGG
and
HD NI NH NI NH HD HD NI NH NH NI NH NG HD HD NG NH
NH;
(SEQ ID NO: 32)
TCCTTCAGAGCCAGGAGTC
and
HD HD NG NG HD NI NH NI NH HD HD NI NH NH NI NH NG
HD;
(SEQ ID NO: 33)
TCCTCCTTCAGAGCCAGGA
and
HD HD NG HD HD NG NG HD NI NH NI NH HD HD NI NH NH
NI;
(SEQ ID NO: 34)
TCCAGCCCCTCCTCCTTCA
and
HD HD NI NH HD HD HD HD NG HD HD NG HD HD NG NG HD
NI;
(SEQ ID NO: 35)
TCCGAGCTTGACCCTTGGA
and
HD HD NH NI NH HD NG NG NH NI HD HD HD NG NG NH NH
NI;
(SEQ ID NO: 36)
TGGTTTCCGAGCTTGACCC
and
NH NH NG NG NG HD HD NH NI NH HD NG NG NH NI HD HD
HD;
(SEQ ID NO: 37)
TGGGGTGGTTTCCGAGCTT
and
NH NH NH NH NG NH NH NG NG NG HD HD NH NI NH HD NG
NG;
(SEQ ID NO: 38)
TCTGCTGGGGTGGTTTCCG
and
HD NG NH HD NG NH NH NH NH NG NH NH NG NG NG HD
HD NH;
(SEQ ID NO: 39)
TGCAGAGTATCTGCTGGGG
and
NH HD NI NH NI NH NG NI NG HD NG NH HD NG NH NH NH
NH;
(SEQ ID NO: 40)
CCAATCCCCTCAGT
and
HD HD NI NI NG HD HD HD HD NG HD NI NH NG;
(SEQ ID NO: 41)
CAGTGCTCAGTGGAA
and
HD NI NH NG NH HD NG HD NI NH NG NH NH NI NI;
(SEQ ID NO: 42)
GAAACATCCGGCGACTCA
and
NH NI NI NI HD NI NG HD HD NH NH HD NH NI HD NG HD
NI;
(SEQ ID NO: 43)
TCGCCCCTCAAATCTTACA
and
HD NH HD HD HD HD NG HD NI NI NI NG HD NG NG NI HD
NI;
(SEQ ID NO: 44)
TCAAATCTTACAGCTGCTC
and
HD NI NI NI NG HD NG NG NI HD NI NH HD NG NH HD NG
HD;
(SEQ ID NO: 45)
TCTTACAGCTGCTCACTCC
and
HD NG NG NI HD NI NH HD NG NH HD NG HD NI HD NG HD
HD;
(SEQ ID NO: 46)
TACAGCTGCTCACTCCCCT
and
NI HD NI NH HD NG NH HD NG HD NI HD NG HD HD HD HD
NG;
(SEQ ID NO: 47)
TGCTCACTCCCCTGCAGGG
and
NH HD NG HD NI HD NG HD HD HD HD NG NH HD NI NH NH
NH;
(SEQ ID NO: 48)
TCCCCTGCAGGGCAACGCC
and
HD HD HD HD NG NH HD NI NH NH NH HD NI NI HD NH HD
HD;
(SEQ ID NO: 49)
TGCAGGGCAACGCCCAGGG
and
NH HD NI NH NH NH HD NI NI HD NH HD HD HD NI NH NH
NH;
(SEQ ID NO: 50)
TCTCGATTATGGGGGGAT
and
HD NG HD NH NI NG NG NI NG NH NH NH HD NH NH NH NI
NG;
(SEQ ID NO: 51)
TCGCTTCTCGATTATGGGC
and
HD NH HD NG NG HD NG HD NH NI NG NG NI NG NH NH NH
HD;
(SEQ ID NO: 52)
TGTCGAGTCGCTTCTCGAT
and
NH NG HD NH NI NH NG HD NH HD NG NG HD NG HD NH NI
NG;
(SEQ ID NO: 53)
TCCATGTCGAGTCGCTTCT
and
HD HD NI NG NH NG HD NH NI NH NG HD NH HD NG NG HD
NG;
(SEQ ID NO: 54)
TCGCCTCCATGTCGAGTCG
and
HD NH HD HD NG HD HD NI NG NH NG HD NH NI NH NG HD
NH;
(SEQ ID NO: 55)
TCGTCATCGCCTCCATGTC
and
HD NH NG HD NI NG HD NH HD HD NG HD HD NI NG NH NG
HD;
(SEQ ID NO: 56)
TGATCTCGTCATCGCCTCC
and
NH NI NG HD NG HD NH NG HD NI NG HD NH HD HD NG HD
HD;
(SEQ ID NO: 57)
GCTTCAGCTTCCTA
and
NH HD NG NG HD NI NH HD NG NG HD HD NG NI;
(SEQ ID NO: 58)
CTGTGATCATGCCA
and
HD NG NK NG NH NI NG HD NI NG NH HD HD NI;
(SEQ ID NO: 59)
ACAGTGGTACACACCT
and
NI HD NI NN NG NN NN NG NI HD NI HD NI HD HD NG;
(SEQ ID NO: 60)
CCACCCCCCACTAAG
and
HD HD NI HD HD HD HD HD HD NI HD NG NI NI NN;
(SEQ ID NO: 61)
CATTGGCCGGGCAC
and
HD NI NG NG NN NN HD HD NN NN NN HD NI HD;
(SEQ ID NO: 62)
GCTTGAACCCAGGAGA
and
NN HD NG NG NN NI NI HD HD HD NI NN NN NI NN NI;
(SEQ ID NO: 63)
ACACCCGATCCACTGGG
and
NI HD NI HD HD HD NN NI NG HD HD NI HD NG NN NN
NN;
(SEQ ID NO: 64)
GCTGCATCAACCCC
and
NN HD NG NN HD NI NG HD NI NI HD HD HD HD;
(SEQ ID NO: 65)
GCCACAAACAGAAATA
and
NN NN HD NI HD NN NI NI NI HD NI HD HD HD NG HD HD;
(SEQ ID NO: 66)
GGTGGCTCATGCCTG
and
NN NN NG NN NN HD NG HD NI NG NN HD HD NG NN;
(SEQ ID NO: 67)
GATTTGCACAGCTCAT
and
NN NI NG NG NG NN HD NI HD NI NN HD NG HD NI NG;
(SEQ ID NO: 68)
AAGCTCTGAGGAGCA
and
NI NI NH HD NG HD NG NH NI NH NH NI NH HD;
(SEQ ID NO: 69)
CCCTAGCTGTCCC
and
HD HD HD NG NI NK HD NG NH NG HD HD HD HD;
(SEQ ID NO: 70)
GCCTAGCATGCTAG
and
NH HD HD NG NI NH HD NI NG NH HD NG NI NH;
(SEQ ID NO: 71)
ATGGGCTTCACGGAT
and
NI NG NH NH NH HD NG NG HD NI HD NH NH NI NG;
(SEQ ID NO: 72)
GAAACTATGCCTGC
and
NH NI NI NI HD NG NI NG NH HD HD NG NH HD;
(SEQ ID NO: 73)
GCACCATTGCTCCC
and
HD
NH NI HD HD NI NG NG NH HD NG HD HD HD;
(SEQ ID NO: 74)
GACATGCAACTCAG
and
NH NI HD NI NG NH HD NI NI HD NG HD NI NH;
(SEQ ID NO: 75)
ACACCACTAGGGGT
and
NI HD NI HD HD NI HD NG NI NH NH NH NH NG;
(SEQ ID NO: 76)
GTCTGCTAGACAGG
and
NH NG HD NG NH HD NG NI NH NI HD NI NH NH;
(SEQ ID NO: 77)
GGCCTAGACAGGCTG
and
NH NH HD HD NG NI NH NI HD NI NH NH HD NG NH;
(SEQ ID NO: 78)
GAGGCATTCTTATCG
and
NH NI NH NH HD NI NG NG HD NG NG NI NG HD NH;
(SEQ ID NO: 79)
GCCTGGAAACGTTCC
and
NN HD HD NG NN NN NI NI NI HD NN NG NG HD HD;
(SEQ ID NO: 80)
GTGCTCTGACAATA
and
NN NG NN HD NG HD NG NN NI HD NI NI NG NI;
(SEQ ID NO: 81)
GTTTTGCAGCCTCC
and
NN NG NG NG NG NN HD NI NN HD HD NG HD HD;
(SEQ ID NO: 82)
ACAGCTGTGGAACGT
and
NI HD NI NN HD NG NN NG NN NN NI NI HD NN NG;
(SEQ ID NO: 83)
GGCTCTCTTCCTCCT
and
HD NI NI NN NI HD HD NN NI NN HD NI HD NG NN HD NG
NN;
(SEQ ID NO: 84)
CTATCCCAAAACTCT
and
HD NG NI NG HD HD HD NI NI NI NI HD NG HD NG;
(SEQ ID NO: 85)
GAAAAACTATGTAT
and
NH NI NI NI NI NI HD NG NI NG NH NG NI NG;
(SEQ ID NO: 86)
AGGCAGGCTGGTTGA
and
NI NH NH HD NI NH NH HD NG NH NH NG NG NH NI;
(SEQ ID NO: 87)
CAATACAACCACGC
and
HD NI NI NG NI HD NI NI HD HD NI HD NN HD;
(SEQ ID NO: 88)
ATGACGGACTCAACT
and
NI NG NN NI HD NN NN NI HD NG HD NI NI HD NG;
and
(SEQ ID NO: 89)
CACAACATTTGTAA
and
HD NI HD NI NI HD NI NG NG NG NN NG NI NI.

In embodiments, the GSHS is within about 25, or about 50, or about 100, or about 150, or about 200, or about 300, or about 500 nucleotides of the TA dinucleotide site, or TTAA (SEQ ID NO: 440) tetranucleotide site or ttTTAAaa (SEQ ID NO: 880) octanucleotide site, or taTTAAta (SEQ ID NO: 881) octanucleotide site.

Illustrative DNA binding codes for targeting human genomic safe harbor in areas of open chromatin via TALES, encompassed by various embodiments are provided in TABLE 20.

TABLE 20

| GSHS | ID | Sequence | TALE (DNA binding code) |
|---|---|---|---|
| AAVS1 | 1 | TGGCCGGCCTGACCACTGG | NH NH HD HD NH NH HD HD NG NH |
| | | (SEQ ID NO: 23) | NI HD HD NI HD NG NH NH |
| AAVS1 | 2 | TGAAGGCCTGGCCGGCCTG | NH NI NI NH NH HD HD NG NH NH |
| | | (SEQ ID NO: 24) | HD HD NH NH HD HD NG NH |
| AAVS1 | 3 | TGAGCACTGAAGGCCTGGC | NH NI NH HD NI HD NG NH NI NI |
| | | (SEQ ID NO: 25) | NH NH HD HD NG NH NH HD |
| AAVS1 | 4 | TCCACTGAGCACTGAAGGC | HD HD NI HD NG NH NI NH HD NI |
| | | (SEQ ID NO: 26) | HD NG NH NI NI NH NH HD |
| AAVS1 | 5 | TGGTTTCCACTGAGCACTG | NH NH NG NG NG HD HD NI HD NG |
| | | (SEQ ID NO: 27) | NH NI NH HD NI HD NG NH |
| AAVS1 | 6 | TGGGGAAAATGACCCAACA | NH NH NH NH NI NI NI NI NG NH |
| | | (SEQ ID NO: 28) | NH HD HD HD NI NI HD NI |
| AAVS1 | 7 | TAGGACAGTGGGGAAAATG | NI NH NH NI HD NI NH NG NH NH |
| | | (SEQ ID NO: 29) | NH NH NI NI NI NI NG NH |
| AAVS1 | 8 | TCCAGGGACACGGTGCTAG | HD HD NI NH NH NH NI HD NI HD |
| | | (SEQ ID NO: 30) | NH NH NG NH HD NG NI NH |
| AAVS1 | 9 | TCAGAGCCAGGAGTCCTGG | HD NI NH NI NH HD HD NI NH NH |
| | | (SEQ ID NO: 31) | NI NH NG HD HD NG NH NH |
| AAVS1 | 10 | TCCTTCAGAGCCAGGAGTC | HD HD NG NG HD NI NH NI NH HD |
| | | (SEQ ID NO: 32) | HD NI NH NH NI NH NG HD |
| AAVS1 | 11 | TCCTCCTTCAGAGCCAGGA | HD HD NG HD HD NG NG HD NI NH |
| | | (SEQ ID NO: 33) | NI NH HD HD NI NH NH NI |
| AAVS1 | 12 | TCCAGCCCCTCCTCCTTCA | HD HD NI NH HD HD HD HD NG HD |
| | | (SEQ ID NO: 34) | HD NG HD HD NG NG HD NI |
| AAVS1 | 13 | TCCGAGCTTGACCCTTGGA | HD HD NH NI NH HD NG NG NH NI |
| | | (SEQ ID NO: 35) | HD HD HD NG NG NH NH NI |
| AAVS1 | 14 | TGGTTTCCGAGCTTGACCC | NH NH NG NG NG HD HD NH NI NH |
| | | (SEQ ID NO: 36) | HD NG NG NH NI HD HD HD |
| AAVS1 | 15 | TGGGGTGGTTTCCGAGCTT | NH NH NH NH NG NH NH NG NG NG |
| | | (SEQ ID NO: 37) | HD HD NH NI NH HD NG NG |
| AAVS1 | 16 | TCTGCTGGGGTGGTTTCCG | HD NG NH HD NG NH NH NH NH NG |
| | | (SEQ ID NO: 38) | NH NH NG NG NG HD HD NH |
| AAVS1 | 17 | TGCAGAGTATCTGCTGGGG | NH HD NI NH NI NH NG NI NG HD |
| | | (SEQ ID NO: 39) | NG NH HD NG NH NH NH NH |
| AAVS1 | AVS1 | CCAATCCCCTCAGT | HD NI NH NG NG HD HD HD HD NG |
| | | (SEQ ID NO: 40) | HD HD NI NI |
| AAVS1 | AVS2 | CAGTGCTCAGTGGAA | HD NI NH NG NH HD NG HD NI NH |
| | | (SEQ ID NO: 41) | NG NH NH NI NI |
| AAVS1 | AVS3 | GAAACATCCGGCGACTCA | NH NI NI NI HD NI NG HD HD NH |
| | | (SEQ ID NO: 42) | NH HD NH NI HD NG HD NI |
| HROSA26 | 1F | TCGCCCCTCAAATCTTACA | HD NH HD HD HD HD NG HD NI NI |
| | | (SEQ ID NO: 43) | NI NG HD NG NG NI HD NI |
| HROSA26 | 2F | TCAAATCTTACAGCTGCTC | HD NI NI NI NG HD NG NG NI HD |
| | | (SEQ ID NO: 44) | NI NH HD NG NH HD NG HD |
| HROSA26 | 3F | TCTTACAGCTGCTCACTCC | HD NG NG NI HD NI NH HD NG NH |
| | | (SEQ ID NO: 45) | HD NG HD NI HD NG HD HD |
| HROSA26 | 4F | TACAGCTGCTCACTCCCCT | NI HD NI NH HD NG NH HD NG HD |
| | | (SEQ ID NO: 46) | NI HD NG HD HD HD HD NG |
| HROSA26 | 5F | TGCTCACTCCCCTGCAGGG | NH HD NG HD NI HD NG HD HD HD |
| | | (SEQ ID NO: 47) | HD NG NH HD NI NH NH NH |
| HROSA26 | 6F | TCCCCTGCAGGGCAACGCC | HD HD HD HD NG NH HD NI NH NH |
| | | (SEQ ID NO: 48) | NH HD NI NI NH HD HD HD |
| HROSA26 | 7F | TGCAGGGCAACGCCCAGGG | NH HD NI NH NH NH HD NI NI HD |
| | | (SEQ ID NO: 49) | NH HD HD HD NI NH NH NH |
| HROSA26 | 8R | TCTCGATTATGGGCGGGAT | HD NG HD NH NI NG NG NI NG NH |
| | | (SEQ ID NO: 50) | NH NH HD NH NH NH NI NG |
| HROSA26 | 9R | TCGCTTCTCGATTATGGGC | HD NH HD NG NG HD NG HD NH NI |
| | | (SEQ ID NO: 51) | NG NG NI NG NH NH NH HD |
| HROSA26 | 10R | TGTCGAGTCGCTTCTCGAT | NH NG HD NH NI NH NG HD NH HD |
| | | (SEQ ID NO: 52) | NG NG HD NG HD NH NI NG |
| HROSA26 | 11R | TCCATGTCGAGTCGCTTCT | HD HD NI NG NH NG HD NH NI NH |
| | | (SEQ ID NO: 53) | NG HD NH HD NG NG HD NG |
| HROSA26 | 12R | TCGCCTCCATGTCGAGTCG | HD NH HD HD NG HD HD NI NG NH |
| | | (SEQ ID NO: 54) | NG HD NH NI NH NG HD NH |
| HROSA26 | 13R | TCGTCATCGCCTCCATGTC | HD NH NG HD NI NG HD NH HD HD |
| | | (SEQ ID NO: 55) | NG HD HD NI NG NH NG HD |
| HROSA26 | 14R | TGATCTCGTCATCGCCTCC | NH NI NG HD NG HD NH NG HD NI |
| | | (SEQ ID NO: 56) | NG HD NH HD HD HD NG HD HD |
| HROSA26 | ROSA1 | GCTTCAGCTTCCTA | NH HD NG NG HD NI NH HD NG NG |
| | | (SEQ ID NO: 57) | HD HD NG NI |
| HROSA26 | ROSA2 | CTGTGATCATGCCA | HD NG NK NG NH NI NG HD NI NG |
| | | (SEQ ID NO: 58) | NH HD HD NI |
| HROSA26 | TALER2 | ACAGTGGTACACACCT | NI HD NI NN NG NN NN NG NI HD |
| | | (SEQ ID NO: 59) | NI HD NI HD HD NG |
| HROSA26 | TALER3 | CCACCCCCCACTAAG | HD HD NI HD HD HD HD HD HD NI |
| | | (SEQ ID NO: 60) | HD NG NI NI NN |
| HROSA26 | TALER4 | CATTGGCCGGGCAC | HD NI NG NG NN NN HD HD NN NN |
| | | (SEQ ID NO: 61) | NN HD NI HD |
| HROSA26 | TALER5 | GCTTGAACCCAGGAGA | NN HD NG NG NN NI NI HD HD HD |
| | | (SEQ ID NO: 62) | NI NN NN NI NN NI |
| CCR5 | TALC3 | ACACCCGATCCACTGGG | NI HD NI HD HD HD NN NI NG HD |
| | | (SEQ ID NO: 63) | HD NI HD NG NN NN NN |
| CCR5 | TALC4 | GCTGCATCAACCCC | NN HD NG NN HD NI NG HD NI NI |
| | | (SEQ ID NO: 64) | HD HD HD HD |
| CCR5 | TALC5 | GCCACAAACAGAAATA | NN NN HD NI HD NN NI NI NI HD |
| | | (SEQ ID NO: 65) | NI HD HD HD NG HD HD |
| CCR5 | TALC7 | GGTGGCTCATGCCTG | NN NN NG NN NN HD NG HD NI NG |
| | | (SEQ ID NO: 66) | NN HD HD NG NN |
| CCR5 | TALC8 | GATTTGCACAGCTCAT | NN NI NG NG NG NN HD NI HD NI |
| | | (SEQ ID NO: 67) | NN HD NG HD NI NG |
| CHR 2 | SHCHR2-1 | AAGCTCTGAGGAGCA | NI NI NH HD NG HD NG NH NI NH |
| | | (SEQ ID NO: 68) | NH NI NH HD |
| CHR 2 | SHCHR2-2 | CCCTAGCTGTCCC | HD HD HD NG NI NK HD NG NH NG |
| | | (SEQ ID NO: 69) | HD HD HD HD |
| CHR 2 | SHCHR2-3 | GCCTAGCATGCTAG | NH HD HD NG NI NH HD NI NG NH |
| | | (SEQ ID NO: 70) | HD NG NI NH |
| CHR 2 | SHCHR2-4 | ATGGGCTTCACGGAT (SEQ | NI NG NH NH NH HD NG NG HD NI |
| | | ID NO: 71) | HD NH NH NI NG |
| CHR 4 | SHCHR4-1 | GAAACTATGCCTGC (SEQ | NH NI NI NI HD NG NI NG NH HD |
| | | ID NO: 72) | HD NG NH HD |
| CHR 4 | SHCHR4-2 | GCACCATTGCTCCC (SEQ | NH HD NI HD HD NI NG NG NH HD |
| | | ID NO: 73) | NG HD HD HD |
| CHR 4 | SHCHR4-3 | GACATGCAACTCAG (SEQ | NH NI HD NI NG NH HD NI NI HD |
| | | ID NO: 74) | NG HD NI NH |
| CHR 6 | SHCHR6-1 | ACACCACTAGGGGT (SEQ | NI HD NI HD HD NI HD NG NI NH |
| | | ID NO: 75) | NH NH NH NG |
| CHR 6 | SHCHR6-2 | GTCTGCTAGACAGG (SEQ | NH NG HD NG NH HD NG NI NH NI |
| | | ID NO: 76) | HD NI NH NH |
| CHR 6 | SHCHR6-3 | GGCCTAGACAGGCTG (SEQ | NH NH HD HD NG NI NH NI HD NI |
| | | ID NO: 77) | NH NH HD NG NH |
| CHR 6 | SHCHR6-4 | GAGGCATTCTTATCG (SEQ | NH NI NH NH HD NI NG NG HD NG |
| | | ID NO: 78) | NG NI NG HD NH |
| CHR 10 | SHCHR10-1 | GCCTGGAAACGTTCC (SEQ | NN HD HD NG NN NN NI NI NI HD |
| | | ID NO: 79) | NN NG NG HD HD |
| CHR 10 | SHCHR10-2 | GTGCTCTGACAATA (SEQ | NN NG NN HD NG HD NG NN NI HD |
| | | ID NO: 80) | NI NI NG NI |
| CHR 10 | SHCHR10-3 | GTTTTGCAGCCTCC (SEQ | NN NG NG NG NG NN HD NI NN HD |
| | | ID NO: 81) | HD NG HD HD |
| CHR 10 | SHCHR10-4 | ACAGCTGTGGAACGT (SEQ | NI HD NI NN HD NG NN NG NN NN |
| | | ID NO: 82) | NI NI HD NN NG |
| CHR 10 | SHCHR10-5 | GGCTCTCTTCCTCCT (SEQ | HD NI NI NN NI HD HD NN NI NN |
| | | ID NO: 83) | HD NI HD NG NN HD NG NN |
| CHR 11 | SHCHR11-1 | CTATCCCAAAACTCT (SEQ | HD NG NI NG HD HD HD NI NI NI |
| | | ID NO: 84) | NI HD NG HD NG |
| CHR 11 | SHCHR11-2 | GAAAAACTATGTAT (SEQ | NH NI NI NI NI NI HD NG NI NG |
| | | ID NO: 85) | NH NG NI NG |
| CHR 11 | SHCHR11-3 | AGGCAGGCTGGTTGA (SEQ | NI NH NH HD NI NH NH HD NG NH |
| | | ID NO: 86) | NH NG NG NH NI |
| CHR 17 | SHCHR17-1 | CAATACAACCACGC (SEQ | HD NI NI NG NI HD NI NI HD HD |
| | | ID NO: 87) | NI HD NN HD |
| CHR 17 | SHCHR17-2 | ATGACGGACTCAACT (SEQ | NI NG NN NI HD NN NN NI HD NG |
| | | ID NO: 88) | HD NI NI HD NG |
| CHR 17 | SHCHR17-3 | CACAACATTTGTAA (SEQ | HD NI HD NI NI HD NI NG NG NG |
| | | ID NO: 89) | NN NG NI NI |
| CHR 17 | SHCHR17-4 | ATTTCCAGTGCACA (SEQ | NI NG NG NG HD HD NI NN NG NN |
| | | ID NO: 90) | HD NI HD NI |

In embodiments, the GSHS is selected from TALES in TABLES 8-12. Further illustrative DNA binding codes for targeting human genomic safe harbor in areas of open chromatin via TALES, encompassed by embodiments are provided in TABLES 8-12. In embodiments, the helper enzyme of the present disclosure is capable of inserting a donor DNA at a TA dinucleotide site. In embodiments, the helper enzyme of the present disclosure is capable of inserting a donor DNA at a TTAA (SEQ ID NO: 440) tetranucleotide site, or a ttTTAAaa (SEQ ID NO: 880) octanucleotide site, or a taTTAAta (SEQ ID NO: 881) octanucleotide site.

TABLE 8
TALE sequences targeting the genomic safe harbor site hROSA26 (hg38 chr3: 9,396,132-
9,396,332).

| NAME | DNA SEQUENCE | RVD AMINO ACID CODE |
|---|---|---|
| R1 | TCGCCCCTCAAATCTTACAG (SEQ | HD NH HD HD HD HD NG HD NI NI NI NG HD NG NG NI |
| | ID NO: 660) | HD NI NH |
| R2 | TCAAATCTTACAGCTGCTCA (SEQ | HD NI NI NI NG HD NG NG NI HD NI NH HD NG NH HD |
| | ID NO: 661) | NG HD NI |
| R3 | TCTTACAGCTGCTCACTCCC (SEQ | HD NG NG NI HD NI NH HD NG NH HD NG HD NI HD NG |
| | ID NO: 662) | HD HD HD |
| R4 | TACAGCTGCTCACTCCCCTG (SEQ | NI HD NI NH HD NG NH HD NG HD NI HD NG HD HD HD |
| | ID NO: 663) | HD NG NH |
| R5 | TGCTCACTCCCCTGCAGGGC (SEQ | NH HD NG HD NI HD NG HD HD HD HD NG NH HD NI NH |
| | ID NO: 664) | NH NH HD |
| R6 | TCCCCTGCAGGGCAACGCCC (SEQ | HD HD HD HD NG NH HD NI NH NH NH HD NI NI HD NH |
| | ID NO: 665) | HD HD HD |
| R7 | TGCAGGGCAACGCCCAGGGA (SEQ | NH HD NI NH NH NH HD NI NI HD NH HD HD HD NI NH |
| | ID NO: 666) | NH NH NI |
| R8 | TCTCGATTATGGGCGGGATT (SEQ | HD NG HD NH NI NG NG NI NG NH NH NH HD NH NH NH |
| | ID NO: 667) | NI NG NG |
| R9 | TCGCTTCTCGATTATGGGCG (SEQ | HD NH HD NG NG HD NG HD NH NI NG NG NI NG NH NH |
| | ID NO: 668) | NH HD NH |
| R10 | TGTCGAGTCGCTTCTCGATT (SEQ | NH NG HD NH NI NH NG HD NH HD NG NG HD NG HD NH |
| | ID NO: 669) | NI NG NG |
| R11 | TCCATGTCGAGTCGCTTCTC (SEQ | HD HD NI NG NH NG HD NH NI NH NG HD NH HD NG NG |
| | ID NO: 670) | HD NG HD |
| R12 | TCGCCTCCATGTCGAGTCGC (SEQ | HD NH HD HD NG HD HD NI NG NH NG HD NH NI NH NG |
| | ID NO: 671) | HD NH HD |
| R13 | TCGTCATCGCCTCCATGTCG (SEQ | HD NH NG HD NI NG HD NH HD HD NG HD HD NI NG NH |
| | ID NO: 672) | NG HD NH |
| R14 | TGATCTCGTCATCGCCTCCA (SEQ | NH NI NG HD NG HD NH NG HD NI NG HD NH HD HD NG |
| | ID NO: 673) | HD HD NI |

TABLE 9
TALE sequences targeting the genomic safe harbor site AAVS1 (hg38 chr19: 55,112,850-
55,113,324).

| NAME | DNA SEQUENCE | RVD AMINO ACID CODE |
|---|---|---|
| AAV1C | TGGCCGGCCTGACCACTGGG (SEQ | NH NH HD HD NH NH HD HD NG NH NI HD HD |
| | ID NO: 674) | NI HD NG NH NH NH |
| AAV2C | TGAAGGCCTGGCCGGCCTGA (SEQ | NH NI NI NH NH HD HD NG NH NH HD HD NH |
| | ID NO: 675) | NH HD HD NG NH NI |
| AAV3C | TGAGCACTGAAGGCCTGGCC (SEQ | NH NI NH HD NI HD NG NH NI NI NH NH HD |
| | ID NO: 676) | HD NG NH NH HD HD |
| AAV4C | TCCACTGAGCACTGAAGGCC (SEQ | HD HD NI HD NG NH NI NH HD NI HD NG NH |
| | ID NO: 677) | NI NI NH NH HD HD |
| AAV5C | TGGTTTCCACTGAGCACTGA (SEQ | NH NH NG NG NG HD HD NI HD NG NH NI NH |
| | ID NO: 678) | HD NI HD NG NH NI |
| AAV6 | TGGGGAAAATGACCCAACAG (SEQ | NH NH NH NH NI NI NI NI NG NH NI HD HD |
| | ID NO: 679) | HD NI NI HD NI NH |
| AAV7 | TAGGACAGTGGGGAAAATGA (SEQ | NI NH NH NI HD NI NH NG NH NH NH NH NI |
| | ID NO: 680) | NI NI NI NG NH NI |
| AAV8 | TCCAGGGACACGGTGCTAGG (SEQ | HD HD NI NH NH NH NI HD NI HD NH NH NG |
| | ID NO: 681) | NH HD NG NI NH NH |
| AAV9 | TCAGAGCCAGGAGTCCTGGC (SEQ | HD NI NH NI NH HD HD NI NH NH NI NH NG |
| | ID NO: 682) | HD HD NG NH NH HD |
| AAV10 | TCCTTCAGAGCCAGGAGTCC (SEQ | HD HD NG NG HD NI NH NI NH HD HD NI NH |
| | ID NO: 683) | NH NI NH NG HD HD |
| AAV11 | TCCTCCTTCAGAGCCAGGAG (SEQ | HD HD NG HD HD NG NG HD NI NH NI NH HD |
| | ID NO: 684) | HD NI NH NH NI NH |
| AAV12 | TCCAGCCCCTCCTCCTTCAG (SEQ | HD HD NI NH HD HD HD HD NG HD HD NG HD |
| | ID NO: 685) | HD NG NG HD NI NH |
| AAV13C | TCCGAGCTTGACCCTTGGAA (SEQ | HD HD NH NI NH HD NG NG NH NI HD HD HD |
| | ID NO: 686) | NG NG NH NH NI NI |
| AAV14C | TGGTTTCCGAGCTTGACCCT (SEQ | NH NH NG NG NG HD HD NH NI NH HD NG NG |
| | ID NO: 687) | NH NI HD HD HD NG |
| AAV15C | TGGGGTGGTTTCCGAGCTTG (SEQ | NH NH NH NH NG NH NH NG NG NG HD HD NH |
| | ID NO: 688) | NI NH HD NG NG NH |
| AAV16C | TCTGCTGGGGTGGTTTCCGA (SEQ | HD NG NH HD NG NH NH NH NH NG NH NH NG |
| | ID NO: 689) | NG NG HD HD NH NI |
| AAV17C | TGCAGAGTATCTGCTGGGGT (SEQ | NH HD NI NH NI NH NG NI NG HD NG NH HD |
| | ID NO: 690) | NG NH NH NH NH NG |

TABLE 10
TALE sequences targeting a chromosome 4 genomic safe harbor site (hg38 chr4:
30,793,038-30,793,980).

| NAME | DNA SEQUENCE | RVD AMINO ACID CODE |
|---|---|---|
| TALE4- | TCTTCCTAGTATTAAAGT (SEQ ID NO: 691) | HD NG NG HD HD NG NI NH NG |
| R001 | | NI NG NG NI NI NI NH NG |
| TALE4- | TCCTTAATATTACCAGT (SEQ ID NO: 692) | HD HD NG NG NI NI NG NI NG |
| R002 | | NG NI HD HD NI NH NG |
| TALE4- | TACCAAGCTGAAATGACACAAAAGT (SEQ ID | NI HD HD NI NI NH HD NG NH |
| F003 | NO: 693) | NI NI NI NG NH NI HD NI HD |
| | | NI NI NI NI NH NG |
| TALE4- | TGGCTGTGTCACATACCAGCAGAAT (SEQ ID | NH NH HD NG NH NG NH NG HD |
| F004 | NO: 694) | NI HD NI NG NI HD HD NI NH |
| | | HD NI NH NI NI NG |
| TALE4- | TGTTAATTTGAATACAATCACT (SEQ ID NO: | NH NG NG NI NI NG NG NG NH |
| F005 | 695) | NI NI NG NI HD NI NI NG HD |
| | | NI HD NG |
| TALE4- | TGTGTCACATACCAGCAGAAT (SEQ ID NO: | NH NG NH NG HD NI HD NI NG |
| F006 | 696) | NI HD HD NI NH HD NI NH NI |
| | | NI NG |
| TALE4- | TGGTAACTACTAATTT (SEQ ID NO: 697) | NH NH NG NI NI HD NG NI HD |
| R007 | | NG NI NI NG NG NG |
| TALE4- | TGTCACATACCAGCAGAAT (SEQ ID NO: | NH NG HD NI HD NI NG NI HD |
| F008 | 698) | HD NI NH HD NI NH NI NI NG |
| TALE4- | TGTGACACAGCCATCAACAAT (SEQ ID NO: | NH NG NH NI HD NI HD NI NH |
| R009 | 699) | HD HD NI NG HD NI NI HD NI |
| | | NI NG |
| TALE4- | TCCTTTGATGAACAGT (SEQ ID NO: 700) | HD HD NG NG NG NH NI NG NH |
| F010 | | NI NI HD NI NH NG |
| TALE4- | TGTGTGCAATAGCGTTAAAGGAACTACAT (SEQ | NH NG NH NG NH HD NI NI NG |
| F011 | ID NO: 701) | NI NH HD NH NG NG NI NI NI |
| | | NH NH NI NI HD NG NI HD NI |
| | | NG |
| TALE4- | TCTTTCAATAGCCCACT (SEQ ID NO: 702) | HD NG NG NG HD NI NI NG NI |
| F012 | | NH HD HD HD NI HD NG |
| TALE4- | TCTCAAATGACAAGAGCACAGT (SEQ ID NO: | HD NG HD NI NI NI NG NH NI |
| R013 | 703) | HD NI NI NH NI NH HD NI HD |
| | | NI NH NG |
| TALE4- | TACCAGTTAATTAGCACT (SEQ ID NO: 704) | NI HD HD NI NH NG NG NI NI |
| F014 | | NG NG NI NH HD NI HD NG |
| TALE4- | TGTTGTGACCTAAGCCAT (SEQ ID NO: 705) | NH NG NG NH NG NH NI HD HD |
| F015 | | NG NI NI NH HD HD NI NG |
| TALE4- | TCTCATGTTTTAAAGTCAAGAAT (SEQ ID NO: | HD NG HD NI NG NH NG NG NG |
| R016 | 706) | NG NI NI NI NH NG HD NI NI |
| | | NH NI NI NG |
| TALE4- | TCCTGAATTCAGAACAGAT (SEQ ID NO: | HD HD NG NH NI NI NG NG HD |
| F017 | 707) | NI NH NI NI HD NI NH NI NG |
| TALE4- | TAGCATGATGTTTCATGTTGTGACCT (SEQ ID | NI NH HD NI NG NH NI NG NH |
| F018 | NO: 708) | NG NG NG HD NI NG NH NG NG |
| | | NH NG NH NI HD HD NG |
| TALE4- | TGTTTCATGTTGTGACCTAAGCCAT (SEQ ID | NH NG NG NG HD NI NG NH NG |
| F019 | NO: 709) | NG NH NG NH NI HD HD NG NI |
| | | NI NH HD HD NI NG |
| TALE4- | TACAACAGTCTATTTCAT (SEQ ID NO: 710) | NI HD NI NI HD NI NH NG HD |
| F020 | | NG NI NG NG NG HD NI NG |

TABLE 11
TALE sequences targeting a chromosome 22 genomic safe harbor site (hg38 chr22:
35,373,428-35,380,000).

| NAME | DNA SEQUENCE | RVD AMINO ACID CODE |
|---|---|---|
| TALE22F- | TCTTCCTAGTCTCTTCTCTACCCAGT (SEQ ID | HD NG NG HD HD NG NI NH |
| R001 | NO: 711) | NG HD NG HD NG NG HD NG |
| | | HD NG NI HD HD HD NI NH |
| | | NG |
| TALE22- | TACACTCCAGCCTGGGAAACAGAGT (SEQ ID NO: | NI HD NI HD NG HD HD NI |
| F002 | 712) | NH HD HD NG NH NH NH NI |
| | | NI NI HD NI NH NI NH NG |
| TALE22- | TCTTTTCCTTAGGACGGCT (SEQ ID NO: 713) | HD NG NG NG NG HD HD NG |
| F003 | | NG NI NH NH NI HD NH NH |
| | | HD NG |
| TALE22- | TCGCTCAGGCCTGTCAT (SEQ ID NO: 714) | HD NH HD NG HD NI NH NH |
| F004 | | HD HD NG NH NG HD NI NG |
| TALE22- | TCCATATGGAAGACTT (SEQ ID NO: 715) | HD HD NI NG NI NG NH NH |
| F005 | | NI NI NH NI HD NG NG |
| TALE22- | TACCCAGTTAACCACCCT (SEQ ID NO: 716) | NI HD HD HD NI NH NG NG |
| F006 | | NI NI HD HD NI HD HD HD |
| | | NG |
| TALE22- | TGGCGCATGCCTGTAATCCCAGCTACT (SEQ ID | NH NH HD NH HD NI NG NH |
| F007 | NO: 717) | HD HD NG NH NG NI NI NG |
| | | HD HD HD NI NH HD NG NI |
| | | HD NG |
| TALE22- | TATACGAGGAGAAAATTAGCATTCCT (SEQ ID | NI NG NI HD NH NI NH NH |
| F008 | NO: 718) | NI NH NI NI NI NI NG NG |
| | | NI NH HD NI NG NG HD HD |
| | | NG |
| TALE22- | TCTGCCTCCCAGGTTCACGCAAT (SEQ ID NO: | HD NG NH HD HD NG HD HD |
| R009 | 719) | HD NI NH NH NG NG HD NI |
| | | HD NH HD NI NI NG |
| TALE22- | TGCCTTGTCACGTTTTCACAGT (SEQ ID NO: | NH HD HD NG NG NH NG HD |
| F010 | 720) | NI HD NH NG NG NG NG HD |
| | | NI HD NI NH NG |
| TALE22- | TGTCACCTTCTGTATGTGCAACCAT (SEQ ID NO: | NH NG HD NI HD HD NG NG |
| F001A | 721) | HD NG NH NG NI NG NH NG |
| | | NH HD NI NI HD HD NI NG |
| TALE22- | TCTGTATGTGCAACCAT (SEQ ID NO: 722) | HD NG NH NG NI NG NH NG |
| F002A | | NH HD NI NI HD HD NI NG |
| TALE22- | TAGTCAAGCAACAGGAT (SEQ ID NO: 723) | NI NH NG HD NI NI NH HD |
| R03A | | NI NI HD NI NH NH NI NG |
| TALE22- | TCCAAGATAATTCCCCAT (SEQ ID NO: 724) | HD HD NI NI NH NI NG NI |
| F004A | | NI NG NG HD HD HD HD NI |
| | | NG |
| TALE22- | TCTGCAAGATCCTTTT (SEQ ID NO: 725) | HD NG NH HD NI NI NH NI |
| F005A | | NG HD HD NG NG NG NG |
| TALE22- | TGCTATGTAAGGTAGCAAAAAGGTAACCT (SEQ ID | NH HD NG NI NG NH NG NI |
| F006A | NO: 726) | NI NH NH NG NI NH HD NI |
| | | NI NI NI NI NH NH NG NI |
| | | NI HD HD NG |
| TALE22- | TCTCTCTCCTCCTGCT (SEQ ID NO: 727) | HD NG HD NG HD NG HD |
| R007A | | NG HD HD NG NH HD NG |
| TALE22- | TCCAAATGCTATTCTCTCT (SEQ ID NO: 728) | HD HD NI NI NI NG NH HD |
| R008A | | NG NI NG NG HD NG HD NG |
| | | HD NG |
| TALE22- | TGCTGATTCAGCCTCCT (SEQ ID NO: 729) | NH HD NG NH NI NG NG HD |
| R009A | | NI NH HD HD NG HD HD NG |
| TALE22- | TAGAACAGCCCCCCACACAGT (SEQ ID NO: | NI NH NI NI HD NI NH HD |
| F010A | 730) | HD HD HD HD HD NI HD NI |
| | | HD NI NH NG |

TABLE 12
TALE sequences targeting chromosome X (HPRT) (hg38 chrX: 134,475,808-134,476,794).

| NAME | DNA SEQUENCE | RVD AMINO ACID CODE |
|---|---|---|
| TALE | TTTAGCAGATGCATCAGC (SEQ ID NO: 731) | NG NG NI NH HD NI NH NI NG NH |
| F002 | | HD NI NG HD NI NH HD |
| TALE | TGACCAGGGGCATGTCCTGG (SEQ ID NO: 732) | NH NI HD HD NI NH NH NH NH HD |
| F003 | | NI NG NH NG HD HD NG NH NH |
| TALE | TGGTCCACCTACCTGAAAATG (SEQ ID NO: 733) | HD NI NI NH NH NI NH NG NG HD |
| F004 | | NG NH NH HD NG NH NH NH NG HD |
| TALE | TGTCCCACAGGTATTACGGGC (SEQ ID NO: 734) | NH NG HD HD HD NI HD NI NH NH |
| F007 | | NG NI NG NG NI HD NH NH NH HD |
| TALE | TACGGGCCAACCTGACAATAC (SEQ ID NO: 735) | NI HD NH NH NH HD HD NI NI HD |
| F008 | | HD NG NH NI HD NI NI NG NI HD |
| TALE | TGAGCTTTGGGGACTGAAAGA (SEQ ID NO: 736) | NH NI NH HD NG NG NG NH NH NH |
| F009 | | NH NI HD NG NH NI NI NI NH NI |
| TALE | CTGGCATAATCTTTTCCCCCA (SEQ ID NO: 737) | NH NH NH NH NH NI NI NI NI NH |
| R002 | | NI NG NG NI NG NH HD HD NI NH |
| TALE | CCAGCCTCCTGGCCATGTGCA (SEQ ID NO: 738) | NH HD NI HD NI NG NH NH HD HD |
| R003 | | NI NH NH NI NH NH HD NG NH NH |
| TALE | GGCCATGTGCACAGGGGCTGA (SEQ ID NO: 739) | HD NI NH HD HD HD HD NG NH NG |
| R004 | | NH HD NI HD NI NG NH NH HD HD |
| TALE | CTGATATGTGAAGGTTTAGCA (SEQ ID NO: 740) | NH HD NG NI NI NI HD HD NG NG |
| R005 | | HD NI HD NI NG NI NG HD NI NH |
| TALE | TGACCAGGCGTGGTGGCTCAC (SEQ ID NO: 741) | NH NI HD HD NI NH NH HD NH NG |
| R007 | | NH NH NG NH NH HD NG HD NI HD |
| TALE | TATAGACATTTTCACT (SEQ ID NO: 742) | NI NG NI NH NI HD NI NG NG NG |
| F020* | | NG HD NI HD NG |
| TALE | TCTACATTTAACTATCAACCT (SEQ ID NO: 743) | HD NG NI HD NI NG NG NG NI NI |
| F021* | | HD NG NI NG HD NI NI HD HD NG |
| TALE | TCGTGCAAACGTTTGAT (SEQ ID NO: 744) | HD NH NG NH HD NI NI NI HD NH |
| F030* | | NG NG NG NH NG |
| TALE | TACATCAATCCTGTAGGT* (SEQ ID NO: 745) | NI HD NI NG HD NI NI NG HD HD |
| F031* | | NG NH NG NI NH NH NG |
| TALE | TCTATTTTAGTGACCCAAGT (SEQ ID NO: 746) | HD NG NI NG NG NG NG NI NH NG |
| F034* | | NH NI HD HD HD NI NI NH NG |
| TALE | TAGAGTCAAAGCATGTACT (SEQ ID NO: 747) | NI NH NI NH NG HD NI NI NI NH |
| F036* | | HD NI NG NH NG NI HD NG |
| TALE | TCCTACCCATAAGCTCCT (SEQ ID NO: 748) | HD HD NG NI HD HD HD NI NG NI |
| F037* | | NI NH HD NG HD HD NG |
| TALE | TCCCCATCCCCATCAGT (SEQ ID NO: 749) | HD HD HD HD NI NG HD HD HD HD |
| F040* | | NI NG HD NI NH NG |
| TALE | TCTTTAATTCAAGCAAGACTTTAACAAGT (SEQ ID | HD NG NG NG NI NI NG NG HD NI |
| R022* | NO: 750) | NI NH HD NI NI NH NI HD NG NG |
| | | NG NI NI HD NI NI NH NG |
| TALE | TGCAGTCCCCTTTCTT (SEQ ID NO: 751) | NH HD NI NH NG HD HD HD HD NG |
| R033* | | NG NG HD NG NG |
| TALE | TCTGCACAAATCCCCAAAGAT (SEQ ID NO: 752) | HD NG NH HD NI HD NI NI NI NG |
| R035* | | HD HD HD HD NI NI NI NH NI NG |
| TALE | TACATGCTTTGACTCT (SEQ ID NO: 753) | NI HD NI NG NH HD NG NG NG NH |
| R038* | | NI HD NG HD NG |
| TALE | TGGCCAGTTATACTGCCAGCAGCTATAAT (SEQ ID | NH NH HD HD NI NH NG NG NI NG |
| R039* | NO: 754) | NI HD NG NH HD HD NI NH HD NI |
| | | NH HD NG NI NG NI NI NG |

*TALES rear hotspots with 85 and 51 hits.

In embodiments, the zinc finger comprises one of the sequences selected from TABLES 13-17, or variants thereof comprising about 99, about 98, about 97, about 95, about 94, about 93, about 92, about 91, about 90, about 89, about 88, about 87, about 86, about 85, about 84, about 83, about 82, about 81, about 80 percent identity to the sequence. In embodiments, the zinc finger targets one or more sites selected from TABLES 13-17.

TABLE 13
Zinc finger sequences targeting the genomic safe harbor site hROSA26 (hg38 chr3: 9,396,132-
9,396,332).

| hROSA | | | | |
|---|---|---|---|---|
| 26 | | | | |
| TTAA | NAME | TARGET | SCORE | ZFP AMINO ACID CODE |
| 5′ | ZnF3a | TGG GAA GAT | 53.64 | LEPGEKPYKCPECGKSFSQNSTLTEHQRTHTGEKPYKCPECGKSFSQRANLRAHQRTH |
| | | AAA CTA (SEQ | | TGEKPYKCPECGKSFSTSGNLVRHQRTHTGEKPYKCPECGKSFSQSSNLVRHQRTETG |
| | | ID NO: 755) | | EKPYKCPECGKSFSRSDHLTTHQRTHTGKKTS (SEQ ID NO: 768) |
| 5′ | ZnF5a | ACT CCC CTG | 56.25 | LEPGEKPYKCPECGKSFSDSGNLRVHQRTHTGEKPYKCPECGKSFSDPGHLVRHQRTH |
| | | CAG GGC | | TGEKPYKCPECGKSFSRADNLTEHQRTHTGEKPYKCPECGKSFSRNDALTEHQRTETG |
| | | AAC (SEQ ID | | EKPYKCPECGKSFSSKKHLAEHQRTHTGEKPYKCPECGKSFSTHLDLIRHQRTHTGKK |
| | | NO: 756) | | TS (SEQ ID NO: 769) |
| 5′ | ZnF5b | CCC CTG CAG | 56.25 | LEPGEKPYKCPECGKSESDCRDLARHQRTHTGEKPYKCPECGKSFSDSGNLRVHQRTH |
| | | GGC AAC | | TGEKPYKCPECGKSFSDPGHLVRHQRTHTGEKPYKCPECGKSFSRADNLTEHQRTETG |
| | | GCC(SEQ ID | | EKPYKCPECGKSFSRNDALTEHQRTHTGEKPYKCPECGKSFSSKKHLAEHQRTHTGKK |
| | | NO: 757) | | TS (SEQ ID NO: 770) |
| 5′ | ZnF5c | CTG CAG GGC | 60.53 | LEPGEKPYKCPECGKSFSRADNLTEHQRTHTGEKPYKCPECGKSFSDCRDLARHQRTH |
| | | AAC GCC | | TGEKPYKCPECGKSFSDSGNLRVHQRTHTGEKPYKCPECGKSFSDPGHLVRHQRTETG |
| | | CAG(SEQ ID | | EKPYKCPECGKSFSRADNLTEHQRTHTGEKPYKCPECGKSFSRNDALTEHQRTHTGKK |
| | | NO: 758) | | TS (SEQ ID NO: 771) |
| 5′ | ZnF5d | CAG GGC AAC | 53.03 | LEPGEKPYKCPECGKSFSQRAHLERHQRTHTGEKPYKCPECGKSFSRADNSTEHQRTH |
| | | GCC CAG | | TGEKPYKCPECGKSFSDCRDLARHQRTHTGEKPYKCPECGKSFSDSGNLRVHQRTETG |
| | | GGA (SEQ ID | | EKPYKCPECGKSFSDPGHLVRHQRTHTGEKPYKCPECGKSFSRADNLTEHQRTHTGKK |
| | | NO: 759) | | TS (SEQ ID NO: 772) |
| 5′ | ZnF5e | GGC AAC GCC | 57.32 | LEPGEKPYKCPECGKSFSTSHSLTEHQRTHTGEKPYKCPECGKSFSQRAHLERHQRTH |
| | | CAG GGA | | TGEKPYKCPECGKSFSRADNLTEHQRTHTGEKPYKCPECGKSFSDCRDLARHQRTETG |
| | | CCA (SEQ ID | | EKPYKCPECGKSFSDSGNLRVHQRTHTGEKPYKCPECGKSFSDPGHLVRHQRTHTGKK |
| | | NO: 760) | | TS (SEQ ID NO: 773) |
| 5′ | ZnF5f | AAC GCC CAG | 54.99 | LEPGEKPYKCPECGKSFSHRTTLTNHQRTHTGEKPYKCPECGKSFSTSHSLTEHQRTH |
| | | GGA CCA | | TGEKPYKCPECGKSFSQRAHLERHQRTHTGEKPYKCPECGKSFSRADNLTEHQRTETG |
| | | AGT (SEQ ID | | EKPYKCPECGKSFSDCRDLARHQRTHTGEKPYKCPECGKSFSDSGNLRVHQRTHTGKK |
| | | NO: 761) | | TS (SEQ ID NO: 774) |
| 5′ | ZnF5g | GCC CAG GGA | 55.31 | LEPGEKPYKCPECGKSFSREDNLHTHQRTHTGEKPYKCPECGKSFSHRTTLTNHQRTH |
| | | CCA AGT | | TGEKPYKCPECGKSFSTSHSLTEHQRTHTGEKPYKCPECGKSFSQRAHLERHQRTETG |
| | | TAG (SEQ ID | | EKPYKCPECGKSFSRADNLTEHQRTHTGEKPYKCPECGKSFSDCRDLARHQRTHTGKK |
| | | NO: 762) | | TS (SEQ ID NO: 775} |
| 5′ | ZnF5h | CAG GGA CCA | 50.76 | LEPGEKPYKCPECGKSFSSKKHLAEHQRTHTGEKPYKCPECGKSFSREDNLHTHQRTH |
| | | AGT TAG | | TGEKPYKCPECGKSFSHRTTLTNHQRTHTGEKPYKCPECGKSFSTSHSLTEHQRTETG |
| | | CCC (SEQ ID | | EKPYKCPECGKSFSQRAHLERHQRTHTGEKPYKCPECGKSFSRADNLTEHQRTHTGKK |
| | | NO: 763) | | TS (SEQ ID NO: 776) |
| 3′ | ZnF12a | GCC TAG GCA | 59.09 | LEPGEKPYKCPECGKSESQSSNLVRHQRTHTGEKPYKCPECGKSFSQRANLRAHQRTH |
| | | AAA GAA (SEQ | | TGEKPYKCPECGKSESQSGDLRRHQRTHTGEKPYKCPECGKSFSREDNLHTHQRTETG |
| | | ID NO: 764) | | EKPYKCPECGKSESDCRDLARHQRTHTGKKTS (SEQ ID NO: 777) |
| 3′ | ZnF13a | CGC GAG GAG | 57.19 | LEPGEKPYKCPECGKSFSRSDHLTNHQRTHTGEKPYKCPECGKSFSRSDHLTNHQRTH |
| | | GAA AGG | | TGEKPYKCPECGKSFSQSSNLVRHQRTHTGEKPYKCPECGKSFSRSDNLVRHQRTETG |
| | | AGG (SEQ ID | | EKPYKCPECGKSFSRSDNLVRHQRTHTGEKPYKCPECGKSFSHTCHLLEHQRTHTGKK |
| | | NO: 765) | | TS (SEQ ID NO: 778) |
| 3′ | ZnF13b | GAG GAG GAA | 57.80 | LEPGEKPYKCPECGKSFSRSDNLVRHQRTHTGEKPYKCPECGKSFSRSDHLTNHQRTH |
| | | AGG AGG | | TGEKPYKCPECGKSFSRSDHLTNHQRTHTGEKPYKCPECGKSFSQSSNLVRHQRTETG |
| | | GAG (SEQ ID | | EKPYKCPECGKSFSRSDNLVRHQRTHTGEKPYKCPECGKSFSRSDNLVRHQRTHTGKK |
| | | NO: 766) | | TS (SEQ ID NO: 779) |
| 3′ | ZnF13c | GAG GAA AGG | 57.61 | LEPGEKPYKCPECGKSFSDPGHLVRHQRTHTGEKPYKCPECGKSFSRSDNLVRHQRTH |
| | | AGG GAG | | IGEKPYKCPECGKSFSRSDHLINHQRTHTGEKPYKCPECGKSFSRSDHLTNHQRTETG |
| | | GGC (SEQ ID | | EKPYKCPECGKSFSQSSNLVRHQRTHTGEKPYKCPECGKSFSRSDNLVRHQRTHTGKK |
| | | NO: 767) | | TS (SEQ ID NO: 780) |

No Sequences have Target site overlap (TSO). The first and last 4 amino acid residues may be omitted from the amino acid code.
Available on the world wide web at scripps. edu/barbas/zfdesign/searchsequence.php

TABLE 14
Zinc finger sequences targeting the genomic safe harbor site AAVS1 (hg38 chr19:
55,112,850-55,113,324).

| AAVS1 | | | | |
|---|---|---|---|---|
| TTAA | NAME | TARGET | SCORE | ZFP AMINO ACID CODE |
| 5′ | ZnF11a | TAG GAC AGT GGG GAA AAT | 57.08 | LEPGEKPYKCPECGKSFSDCRDLARHQRTHTGEKPYKCPECG |
| | | GAC CCA ACA GCC | | KSFSSPADLTRHQRTHTGEKPYKCPECGKSESTSHSLTEHQR |
| | | (SEQ ID NO: 780) | | THTGEKPYKCPECGKSFSDPGNLVRHQRTHTGEKPYKCPECG |
| | | | | KSFSTTGNLTVHQRTHTGEKPYKCPECGKSFSQSSNLVRHQR |
| | | | | THTGEKPYKCPECGKSFSRSDKLVRHQRTHTGEKPYKCPECG |
| | | | | KSFSHRTTLINHQRTHTGEKPYKCPECGKSFSDPGNLVRHQR |
| | | | | THTGEKPYKCPECGKSFSREDNLHTHQRTHTGKKTS (SEQ |
| | | | | ID NO: 795) |
| 5′ | ZnF10a | AGA GGG AGC CAC GAA AAC | 56.91 | LEPGEKPYKCPECGKSFSQLAHLRAHQRTHTGEKPYKCPECG |
| | | AGA (SEQ ID NO: 731) | | KSFSDSGNLRVHQRTHTGEKPYKCPECGKSFSQSSNLVRHQR |
| | | | | THTGEKPYKCPECGKSFSSKKALTEHQRTHTGEKPYKCPECG |
| | | | | KSFSERSHLREHQRTHTGEKPYKCPECGKSFSRSDKLVRHQR |
| | | | | THTGEKPYKCPECGKSFSQLAHLRAHQRTHTGKKTS (SEQ |
| | | | | ID NO: 796) |
| 3′ | ZnF12b | GCA GAT AGC CAG GAG | 59.97 | LEPGEKPYKCPECGKSFSRSDNLVRHQRTHTGEKPYKCPECG |
| | | (SEQ ID NO: 782) | | KSFSRADNLTEHQRTHTGEKPYKCPECGKSFSERSHLREHQR |
| | | | | THTGEKPYKCPECGKSFSTSGNLVRHQRTHTGEKPYKCPECG |
| | | | | KSFSQSGDLRRHQRTHTGKKTS (SEQ ID NO: 797) |
| 3′ | ZnF13b | AGA TAG CCA GGA GTC CTT | 56.80 | TEPGEKPYKCPECGKSFSTTGALTEHQRTHTGEKPYKCPECG |
| | | (SEQ ID NO: 783) | | KSFSDPGALVRHQRTHTGEKPYKCPECGKSFSQRAHLERHQR |
| | | | | THTGEKPYKCPECGKSFSTSFSLTEHQRTHTGEKPYKCPECG |
| | | | | KSFSREDNLHTHQRTHTGEKPYKCPECGKSFSQLAHLRAHQR |
| | | | | THTGKKTS (SEQ ID NO: 798) |
| 5′ | ZnF14a | CCC AGT GGT CAG GCC GGC | 61.73 | LEPGEKPYKCPECGKSFSDCRDLARHQRTHTGEKPYKCPECG |
| | | CAG GCC | | KSFSRADNLTEHQRTHTGEKPYKCPECGKSFSDPGHLVRHQR |
| | | (SEQ ID NO: 734) | | THTGEKPYKCPECGKSFSDCRDLARHQRTHTGEKPYKCPECG |
| | | | | KSFSRADNLTEHQRTHTGEKPYKCPECGKSFSTSGHLVRHQR |
| | | | | THTGEKPYKCPECGKSFSHRTTLTNHQRTHTGEKPYKCPECG |
| | | | | KSFSSKKHLAEHQRTHTGKKTS (SEQ ID NO: 799) |
| 5′ | ZnF15a | GGC CGG CCA GGC CTT CAG | 58.15 | LEPGEKPYKCPECGKSFSRADNLTEHQRTHTGEKPYKCPECG |
| | | (SEQ ID NO: 785) | | KSFSTTGALTEHQRTHTGEKPYKCPECGKSFSDPGHLVRHQR |
| | | | | THTGEKPYKCPECGKSFSTSFSLTEHQRTHTGEKPYKCPECG |
| | | | | KSFSRSDKLTEHQRTHTGEKPYKCPECGKSFSDPGHLVRHQR |
| | | | | THTGKKTS (SEQ TD NO: 800) |
| 5′ | ZnF16a | AGT GCT CAG TGG AAA CCA | 58.65 | LEPGEKPYKCPECGKSFSDPGNLVRHQRTHTGEKPYKCPECG |
| | | CGA AAG GAC | | KSFSRKDNLKNHQRTHTGEKPYKCPECGKSFSQSGHLTEHQR |
| | | (SEQ ID NO: 786) | | THTGEKPYKCPECGKSFSTSESLTEHQRTHTGEKPYKCPECG |
| | | | | KSFSQRANLRAHQRTHTGEKPYKCPECGKSFSRSDHLTTHQR |
| | | | | THTGEKPYKCPECGKSFSRADNLTEHQRTHTGEKPYKCPECG |
| | | | | KSFSTSGELVRHQRTHTGEKPYKCPECGKSFSHRTTLTNHQR |
| | | | | THTGKKTS (SEQ ID NO: 801) |
| 5′ | ZnF17a | TGG CCC CCA GCC CCT CCT | 60.89 | LEPGEKPYKCPECGKSFSDCRDLARHQRTHTGEKPYKCPECG |
| | | GCC (SEQ ID NO: 787) | | KSFSTKNSLTEHQRTHTGEKPYKCPECGKSFSTKNSLTEHQR |
| | | | | THTGEKPYKCPECGKSFSDCRDLARHQRTHTGEKPYKCPECG |
| | | | | KSFSTSHSLTEHQRTHTGEKPYKCPECGKSFSSKKHLAEHQR |
| | | | | THTGEKPYKCPECGKSFSRSDHLTTHQRTHTGKKTS (SEQ |
| | | | | ID NO: 802) |
| 5′ | ZnF18a | AGA GCC AGG AGT CCT GGC | 57.23 | LEPGEKPYKCPECGKSFSSKKHLAEHQRTHTGEKPYKCPECG |
| | | CCC CAG CCC | | KSFSRADNLTEHQRTHTGEKPYKCPECGKSFSSKKHLAEHQR |
| | | (SEQ ID NO: 788) | | THTGEKPYKCPECGKSFSDPGHLVRHQRTHTGEKPYKCPECG |
| | | | | KSFSTKNSLTEHQRTHTGEKPYKCPECGKSFSHRTTLTNHQR |
| | | | | THTGEKPYKCPECGKSFSRSDHLTNHQRTHTGEKPYKCPECG |
| | | | | KSFSDCRDLARHQRTHTGEKPYKCPECGKSFSQLAHLRAHQR |
| | | | | THTGKKTS (SEQ ID NO: 803) |
| 3′ | ZnF19a | GCA GGA GGG GCT GGG GGC | 59.93 | LEPGEKPYKCPECGKSFSDPGNLVRHQRTHTGEKPYKCPECG |
| | | CAG GAC (SEQ ID NO: 739) | | KSFSRADNLTEHQRTHTGEKPYKCPECGKSFSDPGHLVRHQR |
| | | | | THTGEKPYKCPECGKSFSRSDKLVRHQRTHTGEKPYKCPECG |
| | | | | KSFSTSGELVRHQRTHTGEKPYKCPECGKSFSRSDKLVRHQR |
| | | | | THTGEKPYKCPECGKSFSQRAHLERHQRTHTGEKPYKCPECG |
| | | | | KSFSQSGDLRRHQRTHTGKKTS (SEQ ID NO: 804) |
| 3′ | ZnF20b | ATA GCC CTG GGC CCA CGG | 59.53 | LEPGEKPYKCPECGKSFSSRRTCRAHQRTHTGEKPYKCPECG |
| | | CTT CGT (SEQ ID NO: 790) | | KSFSTTGALTEHQRTHTGEKPYKCPECGKSFSRSDKLTEHQR |
| | | | | THTGEKPYKCPECGKSFSTSFSLTEHQRTHTGEKPYKCPECG |
| | | | | KSFSDPGHLVRHQRTHTGEKPYKCPECGKSFSRNDALTEHQR |
| | | | | THTGEKPYKCPECGKSFSDCRDLARHQRTHTGEKPYKCPECG |
| | | | | KSFSQKSSLIAHQRTHTGKKT (SEQ ID NO: 805) |
| 3′ | ZnF21b | GAA GGA CCT GGC TGG | 55.22 | LEPGEKPYKCPECGKSFSRSDHLTTHQRTHTGEKPYKCPECG |
| | | (SEQ ID NO: 791) | | KSFSDPGHLVRHQRTHTGEKPYKCPECGKSFSTKNSLTEHQR |
| | | | | THTGEKPYKCPECGKSFSCRAHLERHQRTHTGEKPYKCPECG |
| | | | | KSFSQSSNTVRHQRTHTAKKTS (SEQ ID NO: 806) |
| 5′ | ZnF22a | GCA GGA ACG AAG CCG TGG | 56.47 | LEPGEKPYKCPECGKSFSDPGHLVRHQRTHTGEKPYKCPECG |
| | | GCC CAG GGC | | KSFSRADNLTEHQRTHTGEKPYKCPECGKSESDQRDLARHQR |
| | | (SEQ ID NO: 792) | | THTGEKPYKCPECGKSFSRSDHLTTHQRTHTGEKPYKCPECG |
| | | | | KSFSRNDTLTEHQRTHTGEKPYKCPECGKSFSRKDNLKNHQR |
| | | | | THTGEKPYKCPECGKSFSRTDTLRDHQRTHTGEKPYKCPECG |
| | | | | KSFSQRAHLERHQRTHTGEKPYKCPECGKSFSQSGDLRRHQR |
| | | | | THTGKKTS (SEQ ID NO: 807) |
| 5′ | ZnF23a | GGA AAC CAC CCC AGC AGA | 52.63 | LEPGEKPYKCPECGKSFSQLAHLRAHQRTHTGEKPYKCPECG |
| | | (SEQ ID NO: 793) | | KSFSERSHLREHQRTHTGEKPYKCPECGKSFSSKKHLAEHQR |
| | | | | THIGEKPYKCPECGKSFSSKKALTEHQRTHTGEKPYKCPECG |
| | | | | KSFSDSGNLRVHQRTHTGEKPYKCPECGKSFSQRAHLERHQR |
| | | | | THTGKKTS (SEQ ID NO: 808) |
| 5′ | ZnF24a | AAG GGT CAA GCT CGG AAA | 55.09 | LEPGEKPYKCPECGKSFSQKSSLIAHQRTHTGEKPYKCPECG |
| | | CCA CCC CAG CAG ATA | | KSFSRADNLTEHQRTHTGEKPYKCPECGKSFSRADNLTEHQR |
| | | (SEQ ID NO: 794) | | THTGEKPYKCPECGKSFSSKKHLAEHQRTHTGEKPYKCPECG |
| | | | | KSFSTSHSLTEHQRTHTGEKPYKCPECGKSFSQRANLRAHQR |
| | | | | THTGEKPYKCPECGKSFSRSDKLTEHQRTHTGEKPYKCPECG |
| | | | | KSFSTSGELVRHQRTHTGEKPYKCPECGKSFSQSGNLTEHQR |
| | | | | THTGEKPYKCPECGKSFSTSGHLVRHQRTHTGEKPYKCPECG |
| | | | | KSFSRKDNLKNHQRTHTGKKTS (SEQ ID NO: 809) |

No Sequences have Target. site overlap (TSO). The first and last. 4 amino acid residues may be omitted from the amino acid code.
Available on the world wide web at scripps. edu/barbas/zfdesign/searchsequence.php

TABLE 15
Zinc finger sequences targeting a chromosome 4 genomic safe harbor site (hg38 chr4:
30,793,038-30,793,980).

| Chr4 | | | | |
|---|---|---|---|---|
| TTAA | NAME | TARGET | SCORE | ZFP AMINO ACID CODE |
| 5′ | ZnF31F | CTTTGATGAACAGTCACA (SEQ | 58.41 | LEPGEKPYKCPECGKSFSSPADLTRHQRTHTGEKPYKCPECG |
| | | ID NO: 810) | | KSFSDPGALVRHQRTHTGEKPYKCPECGKSFSSPADLTRHQR |
| | | | | THTGEKPYKCPECGKSFSCAGHLASHQRTHTGEKPYKCPECG |
| | | | | KSFSQAGHLASHQRTHTGEKPYKCPECGKSFSTTGALTEHQR |
| | | | | THTGKKTS (SEQ ID NO: 819) |
| 5′ | ZnF32F | CTTCCAATTAGTCCTACC (SEQ | 55.84 | LEPGEKPYKCPECGKSFSDKKDLTRHQRTHTGEKPYKCPECG |
| | | ID NO: 811) | | KSFSTKNSLTEHQRTHTGEKPYKCPECGKSFSHRTTLTNHQR |
| | | | | THTGEKPYKCPECGKSFSHKNALQNHQRTHTGEKPYKCPECG |
| | | | | KSFSTSHSLTEHQRTHTGEKPYKCPECGKSFSTTGALTEHQR |
| | | | | THTGKKTS (SEQ ID NO: 820) |
| 5′ | ZnF33F | ATACTAGGAAGAAATACAATA | 57.27 | LEPGEKPYKCPECGKSFSCKSSLIAHQRTHTGEKPYKCPECG |
| | | (SEQ ID NO: 812) | | KSFSSPADLTRHQRTHTGEKPYKCPECGKSFSTTGNLTVHQR |
| | | | | THTGEKPYKCPFCGKSFSQLAHIRAHQRTHTGEKPYKCPECG |
| | | | | KSFSQRAHLERHQRTHTGEKPYKCPECGKSFSQNSTLTEHQR |
| | | | | THTGEKPYKCPECGKSFSCKSSLIAHQRTHTGKKTS (SEQ |
| | | | | ID NO: 821) |
| 5′ | ZnF34F | GCTCTTGTCATTTGAGAT | 57.38 | LEPGEKPYKCPECGKSFSTSGNLVRHQRTHTGEKPYKCPECG |
| | | (SEQ ID NO: 813) | | KSFSQAGHLASHQRTHTGEKPYKCPECGKSFSHKNALQNHQR |
| | | | | THTGEKPYKCPECGKSFSDPGALVRHQRTHTGEKPYKCPECG |
| | | | | KSFSTTGALTEHQRTHTGEKPYKCPECGKSFSTSGELVRHQR |
| | | | | THTGKKTS (SEQ ID NO: 822) |
| 5 | ZnF35F | CCAAGCTGAAATGACACAAAAGTT | 58.23 | LEPGEKPYKCPECGKSFSRKDNLKNHQRTHTGEKPYKCPECG |
| | | AAAACAAAG | | KSFSSPADLTRHQRTHTGEKPYKCPECGKSFSQRANLRAHQR |
| | | (SEQ ID NO: 814) | | THTGEKPYKCPECGKSFSTSGSLVRHQRTHTGEKPYKCPECG |
| | | | | KSFSQRANLRAHQRTHTGEKPYKCPECGKSFSSPADLTRHQR |
| | | | | THTGEKPYKCPECGKSFSDPGNLVRHQRTHTGEKPYKCPECG |
| | | | | KSFSTTGNLTVHQRTHTGEKPYKCPECGKSFSQAGHLASHQR |
| | | | | THTGEKPYKCPECGKSFSERSHLREHQRTHTGEKPYKCPECG |
| | | | | KSFSTSHSLTEHQRTHTGKKTS (SEQ ID NO: 823) |
| 5′ | ZnF36F | CTTATACCAGTTAATTAGCAC | 49.93 | LEPGEKPYKCPECGKSFSSKKALTEHQRTHTGEKPYKCPECG |
| | | (SEQ ID NO: 815) | | KSFSREDNLHTHQRTHTGEKPYKCPECGKSFSTTGNLTVHQR |
| | | | | THTGEKPYKCPECGKSFSTSGSLVRHQRTHTGEKPYKCPECG |
| | | | | KSFSTSHSLTEHQRTHTGEKPYKCPECGKSFSQKSSLIAHQR |
| | | | | THTGEKPYKCPECGKSFSTTGALTEHQRTHTGKKTS (SEQ |
| | | | | ID NO: 824) |
| 3′ | ZnF37R | AACGCTATTGCACACATAGTTACA | 57.67 | LEPGEKPYKCPECGKSFSSPADLTRHQRTHTGEKPYKCPECG |
| | | (SEQ ID NO: 816) | | KSFSTSGSLVRHQRTHTGEKPYKCPECGKSFSQKSSLIAHQR |
| | | | | THTGEKPYKCPECGKSFSSKKALTEHQRTHTGEKPYKCPECG |
| | | | | KSFSQSGDLRRHQRTHTGEKPYKCPECGKSFSHKNALQNHQR |
| | | | | THTGEKPYKCPECGKSFSTSGELVRHQRTHTGEKPYKCPECG |
| | | | | KSFSDSGNLRVIIQRTIITGKKTS (SEQ ID NO: 825) |
| 3. | ZnF38R | TGAATTCAGGAACAAAGTATA | 53.21 | LEPGEKPYKCPECGKSFSQKSSLIAHQRTHTGEKPYKCPECG |
| | | (SEQ ID NO: 817) | | KSFSHRTTLINHQRTHTGEKPYKCPECGKSFSQSGNLTEHQR |
| | | | | THTGEKPYKCPECGKSFSQSSNLVRHQRTHTGEKPYKCPECG |
| | | | | KSFSRADNLTEHQRTHTGEKPYKCPECGKSFSHKNALQNHQR |
| | | | | THATGEKPYKCPECGKSFSCAGHLASHQRTHTGKKTS (SEQ |
| | | | | ID NO: 826) |
| 3′ | ZnF39R | GCTGGTATGTGACACAGCCATCAA | 50.63 | LEPGEKPYKCPECGKSFSQSGNLTEHQRTHTGEKPYKCPECG |
| | | CAA(SEQ ID NO: 818) | | KSFSQSGNLTEHQRTHTGEKPYKCPECGKSESTSGNLTEHQR |
| | | | | THTGEKPYKCPECGKSFSERSHLREHQRTHTGEKPYKCPECG |
| | | | | KSFSSKKALTEHQRTHTGEKPYKCPECGKSFSQAGHLASHQR |
| | | | | THTGEKPYKCPECGKSFSRRDELNVHQRTHTGEKPYKCPECG |
| | | | | KSFSTSGHLVRHQRTHTGEKPYKCPECGKSFSTSGELVRHQR |
| | | | | THTGKKTS (SEQ ID NO: 827) |

No Sequences have Target site overlap (TSO). The first and last 4 amino acid residues may be omitted from the amino acid code.
Available on the world wide web at scripps. edu/barbas/zfdesign/searchsequence.php

TABLE 16
Zinc finger sequences targeting a chromosome 22 genomic safe harbor site (hg38 chr22:
35,373,428-35,380,000).

| Chr22 | | | | |
|---|---|---|---|---|
| TTAA | NAME | TARGET | SCORE | ZFP |
| 5′ | ZnF1a | CTTCCTGAAAGCAAGA | 57.34 | LEPGEKPYKCPECGKSFSTTGNLTVHQRTHTGEKPYKCPECGKSFSQAGHLASH |
| | | GATGAAAT (SEQ | | QRTHTGEKPYKCPECGKSFSQLAHLRAHQRTHTGEKPYKCPECGKSFSRKDNLK |
| | | ID NO: 828) | | NIQRTHTGEKPYKCPECGKSFSERSHLREHQRTHTGEKPYKCPECGKSFSQSSN |
| | | | | LVRHQRTHTGEKPYKCPECGKSFSTKNSLTEHQRTHTGEKPYKCPECGKSFSTT |
| | | | | GALTEHQRTHTGKKTS (SEQ ID NO: 845) |
| 5′ | ZnF1b | CTGAAAGCAAGAGATG | 58.92 | LEPGEKPYKCPECGKSFSTSHSLTEHQRTHTGEKPYKCPECGKSFSHKNALQNH |
| | | AAATTCCA (SEQ ID | | QRTHTGEKPYKCPECGKSFSQSSNLVRHQRTHTGEKPYKCPECGKSFSTSGNLV |
| | | NO: 829) | | RHQRTHTGEKPYKCPECGKSFSQLAHLRAHQRTHTGEKPYKCPECGKSFSQSGD |
| | | | | LRRHQRTHTGEKPYKCPECGKSFSQRANLRAHQRTHTGEKPYKCPECGKSFSRN |
| | | | | DALTEHQRTHTGKKTS (SEQ ID NO: 846) |
| 5′ | ZnF2a | ATACGAGGAGAAAATT | 51.25 | LEPGEKPYKCPECGKSFSTSGNLTEHQRTHTGEKPYKCPECGKSFSREDNLHTH |
| | | AGCAT (SEQ ID | | QRTHTGEKPYKCPECGKSFSTTGNLTVHQRTHTGEKPYKCPECGKSFSQSSNLV |
| | | NO: 830) | | RHQRTHTGEKPYKCPECGKSFSQRAHLERHQRTHTGEKPYKCPECGKSFSQSGH |
| | | | | LTEHQRTHTGEKPYKCPECGKSFSQKSSTIAHQRTHTGKKTS (SEQ ID NO: |
| | | | | 847) |
| 5 | ZnF3a | CATCCATGGCAGGAAG | 58.67 | LEPGEKPYKCPECGKSFSRNDALTEHQRTHTGEKPYKCPECGKSFSTTGNLTVH |
| | | TTGAAGCCAAAATAAA | | QRTHTGEKPYKCPECGKSFSQKSSLIAHQRTHTGEKPYKCPECGKSFSQRANLR |
| | | TCTG (SEQ ID | | AHQRTHTGEKPYKCPECGKSFSDCRDLARHQRTHTGEKPYKCPECGKSFSQSSN |
| | | NO: 831) | | LVRHQRTHTGEKPYKCPECGKSFSTSGSLVRHQRTHTGEKPYKCPECGKSFSQS |
| | | | | SNLVRHQRTHTGEKPYKCPECGKSFSRADNLTEFQRTHTGEKPYKCPECGKSFS |
| | | | | RSDHLTTHQRTHTGEKPYKCPECGKSFSTSHSLTEHQRTHTGEKPYKCPECGKS |
| | | | | FSTSGNLTEHQRTETGKKTS (SEQ ID NO: 848) |
| 5′ | ZnF3b | ATGGCAGGAAGTTGAA | 54.14 | LEPGEKPYKCPECGKSFSQRANLRAHQRTHTGEKPYKCPECGKSFSTTGNLTVH |
| | | GCCAAAAAAA (SEQ | | QRTHTGEKPYKCPECGKSFSQSGNLTEHQRTHTGEKPYKCPECGKSFSERSHLR |
| | | ID NO: 832) | | EHQRTHTGEKPYKCPECGKSFSQAGHLASHQRTHTGEKPYKCPECGKSFSHRTT |
| | | | | LTNHQRTHTGEKPYKCPECGKSFSQRAHLERHQRTHTGEKPYKCPECGKSFSQS |
| | | | | GDLRRHQRTHTGEKPYKCPECGKSFSRRDELNVEQRTHTGKKTS (SEQ ID |
| | | | | NO: 849) |
| 3′ | ZnF5aR | GAAAAGAAGACTCAAG | 55.40 | LEPGEKPYKCPECGKSFSSKKALTEHQRTHTGEKPYKCPECGKSFSQRANLRAH |
| | | GAAACAGAGCCAAACA | | QRTHTGEKPYKCPECGKSFSDCRDLARHQRTHTGEKPYKCPECGKSFSQLAHLR |
| | | C (SEQ ID | | AHQRTHTGEKPYKCPECGKSFSDSGNLRVHQRTHTGEKPYKCPECGKSFSQRAH |
| | | NO: 833) | | LERHQRTHTGEKPYKCPECGKSFSQSGNLTEHQRTHTGEKPYKCPECGKSFSTH |
| | | | | LDLIRHQRTHTGEKPYKCPECGKSFSRKDNLKNEQRTHTGEKPYKCPECGKSFS |
| | | | | RKDNLKNHQRTHTGEKPYKCPECGKSFSQSSNLVRHQRTHTGKKTS (SEQ ID |
| | | | | NO: 850) |
| 3 | ZnF5bR | AGGAAACAGAGCCAAA | 54.66 | LEPGEKPYKCPECGKSFSSPADLTRHQRTHTGEKPYKCPECGKSFSTTGALTEH |
| | | CACTTACA (SEQ ID | | QRTHTGEKPYKCPECGKSFSSPADLTRHQRTHTGEKPYKCPECGKSFSQSGNLT |
| | | NO: 834) | | EHQRTHTGEKPYKCPECGKSFSERSHLREHQRTHTGEKPYKCPECGKSFSRADN |
| | | | | LTEHQRTHTGEKPYKCPECGKSFSQRANLRAHQTHTGEKPYKCPECGKSFSRS |
| | | | | DHLTNHQRTHTGKKTS (SEQ ID NO: 851) |
| 3′ | ZnF6aR | ATGCAGATTTGGACAC | 58.57 | LEPGEKPYKCPECGKSFSRSDKLVRHQRTHTGEKPYKCPECGKSFSSRRTCRAH |
| | | AGAGTAGTAAACTGTG | | QRTHTGEKPYKCPECGKSFSRSDHLTTHQRTHTGEKPYKCPECGKSFSRKDNLK |
| | | AAAACGTGACAAGGCA | | NHQRTHTGEKPYKCPECGKSFSQSGDLRRHQRTHTGEKPYKCPECGKSFSRKDN |
| | | AAGTGGCGTGGG | | LKNHQRTHTGEKPYKCPECGKSFSDPGNLVRHQRTHTGEKPYKCPECGKSFSSR |
| | | (SEQ ID NO: 835) | | RTCRAHQRTHTGEKPYKCPECGKSFSQRANLRAFQRTHTGEKPYKCPECGKSFS |
| | | | | QACHLASHQRTHTGEKPYKCPECGKSFSRNDALTEHQRTHTGEKPYKCPECGKS |
| | | | | FSQRANLRAHQRTETGEKPYKCPECGKSFSHRTTLTNHQRTHTGEKPYKCPECG |
| | | | | KSFSHRTTLTNHQRTHTGEKPYKCPECGKSFSRADNLTEHQRTHTGEKPYKCPE |
| | | | | CGKSFSSPADLTREQRTHTGEKPYKCPECGKSFSRSDHLTTHQRTHTGEKPYKC |
| | | | | PECGKSFSHKNALQNHQRTHTGEKPYKCPECGKSFSRADNLTEHQRTHTGEKPY |
| | | | | KCPECGKSESRRDELNVHQRTHTGKKTS (SEQ ID NO: 852) |
| 3′ | ZnF6bR | GGACACAGAGTAGTAA | 55.80 | LEPGEKPYKCPECGKSFSDSGNLRVHQRTHTGEKPYKCPECGKSFSQSSSLVRH |
| | | AC (SEQ ID | | QRTHTGEKPYKCPECGKSFSQSSSLVRHQRTHTGEKPYKCPECGKSFSQLAHLR |
| | | NO: 836) | | AHQRTHTGEKPYKCPECGKSFSSKKALTEHQRTHTGEKPYKCPECGKSFSQRAH |
| | | | | LERHQRTHTGKKTS (SEQ ID NO: 853) |
| 5′ | ZnF10F | AAAGCTAGCAGCATGG | 57.55 | LEPGEKPYKCPECGKSFSQSGDLRRHQRTHTGEKPYKCPECGKSFSRRDELNVH |
| | | CA (SEQ ID | | QRTHTGEKPYKCPFCGKSFSERSHLREHQRTHTGEKPYKCPECGKSFSFRSHIR |
| | | NO: 837) | | EHQRTHTGEKPYKCPECGKSFSTSGELVRHQRTHTGEKPYKCPECGKSFSQRAN |
| | | | | LRAHQRTHTGKKTS (SEQ ID NO: 854) |
| 5′ | ZnF11F | CCTCTTATAAGGCCCA | 52.55 | LEPGEKPYKCPECGKSFSQKSSLIAHQRTHTGEKPYKCPECGKSFSRSDHLTNH |
| | | AGAGGATA (SEQ | | QRTHTGEKPYKCPECGKSFSRKDNLKNHQRTHTGEKPYKCPECGKSFSSKKHLA |
| | | ID NO: 838) | | EHQRTHTGEKPYKCPECGKSFSRSDHLTNHQRTHTGEKPYKCPECGKSFSQKSS |
| | | | | LIAHQRTHTGEKPYKCPECGKSFSTTGALTEHQRTHTGEKPYKCPECGKSFSTK |
| | | | | NSLTEHQRTHTGKKTS (SEQ ID NO: 855) |
| 5′ | ZnF12F | CAACATCCTTGACTTA | 55.00 | LEPGEKPYKCPECGKSESSKKALTEHQRTHTGEKPYKCPECGKSFSTTGNLTVH |
| | | ATCAC (SEQ ID | | QRTHTGEKPYKCPECGKSFSTTGALTEHQRTHTGEKPYKCPECGKSFSQAGHLA |
| | | NO: 839) | | SHQRTHTGEKPYKCPECGKSFSTKNSLTEHQRTHTGEKPYKCPECGKSFSTSGN |
| | | | | LTEHQRTHTGEKPYKCPECGKSFSQSGNTEHQRTHTGKKTS (SEQ ID NO: |
| | | | | 856) |
| 5′ | ZnF13E | GGTAGCAAAAAGGTAA | 46.33 | LEPGEKPYKCPECGKSFSDKKDLTRHQRTHTGEKPYKCPECGKSFSQSSSLVRH |
| | | CC (SEQ ID | | QRTHTGEKPYKCPECGKSFSRKDNLKNHQRTHTGEKPYKCPECGKSFSQRANLR |
| | | NO: 840) | | AHQRTHTGEKPYKCPECGKSFSERSHLREHQRTHTGEKPYKCPECGKSFSTSGH |
| | | | | LVRHQRTHTGKKTS (SEQ ID NO: 857) |
| 3′ | ZnF14R | TGGGGTGCAAGAGGCC | 61.28 | LEPGEKPYKCPECGKSFSDPGALVRHQRTHTGEKPYKCPECGKSESRNDALTEH |
| | | AGGCCAGAGTTGTTCT | | QRTHTGEKPYKCPECGKSFSTSGSLVRHQRTHTGEKPYKCPECGKSFSTSGSLV |
| | | GGTC (SEQ ID | | RHQRTHTGEKPYKCPECGKSFSQLAHLRAHQRTHTGEKPYKCPECGKSFSDCRD |
| | | NO: 841) | | LARHQRTHTGEKPYKCPECGKSFSRADNLTEHQRTHTGEKPYKCPECGKSFSDP |
| | | | | GHLVRHQRTHTGEKPYKCPECGKSFSQLAHLRAFQRTHTGEKPYKCPECGKSFS |
| | | | | QSGDLRRHQRTHTGEKPYKCPECGKSFSTSGHLVRHQRTHTGEKPYKCPECGKS |
| | | | | FSRSDHLTTHQRTETGKKTS (SEQ ID NO: 858) |
| 3′ | ZnF15R | CGCATGCTGATTCAGC | 58.41 | LEPGEKPYKCPECGKSFSDPGNLVRHQRTHTGEKPYKCPECGKSFSTKNSLTEH |
| | | CTCCTGAC (SEQ ID | | QRTHTGEKPYKCPECGKSFSTKNSLTEHQRTHTGEKPYKCPECGKSFSRADNLT |
| | | NO: 842) | | EHQRTHTGEKPYKCPECGKSFSHKNALQNHQRTHTGEKPYKCPECGKSFSRNDA |
| | | | | LTEHQRTHTGEKPYKCPECGKSFSRRDENVHQRTHTGEKPYKCPECGKSFSHT |
| | | | | GHLLEHQRTHTGKKTS (SEQ ID NO: 859) |
| 3′ | ZnF14R | AGTCAAGCAACAGGAT | 50.89 | LEPGEKPYKCPECGKSFSQAGHLASHQRTHTGEKPYKCPECGKSFSQRAHLERH |
| | | GA (SEQ ID | | QRTHTGEKPYKCPECGKSFSSPADLTRHQRTHTGEKPYKCPECGKSFSQSGDLR |
| | | NO: 843) | | RHQRTHTGEKPYKCPECGKSFSQSGNLTEHQRTHTGEKPYKCPECGKSFSHRTT |
| | | | | LTNHQRTHTGKKTS (SEQ ID NO: 860) |
| 3′ | ZnF15R | GTCAAGCAACAGGATG | 59.22 | LEPGEKPYKCPECGKSFSHKNALQNHQRTHTGEKPYKCPECGKSFSTSGELVRH |
| | | ATCCAAAGCTATT | | QRTHTGEKPYKCPECGKSFSTTGNLTVHQRTHTGEKPYKCPECGKSFSTSHSLT |
| | | (SEQ ID NO: 844) | | EHQRTHTGEKPYKCPECGKSFSTSGNLVRHQRTHTGEKPYKCPECGKSFSTSGN |
| | | | | LVRHQRTHTGEKPYKCPECGKSFSRADNLTEHQRTHTGEKPYKCPECGKSFSQS |
| | | | | GNLTEHQRTHTGEKPYKCPECGKSFSRKDNLKNEQRTHTGEKPYKCPECGKSFS |
| | | | | DPGALVRHQRTHTGKKTS (SEQ ID NO: 861) |

No Sequences have Target site overlap (TSO). The first and last 4 amino acid residues may be omitted from the amino acid code.
Available on the world wide web at scripps. edu/barbas/zfdesign/searchsequence.php

TABLE 17
Zinc finger sequences targeting chromosome X (HPRT) (hg38 chrX: 134,475,808-134,476,794).

| ChrX | | | | |
|---|---|---|---|---|
| TTAA | NAME | TARGET | SCORE | ZFP AMINO ACID CODE |
| 5′ | ZnF41F | GTAGAAACTCGCCTTATG (SEQ ID | 54.04 | LEPGEKPYKCPECGKSFSRRDELNVHQRTHTGEKPYKCPECG |
| | | NO: 862) | | KSFSTTGALTEHQRTHTGEKPYKCPECGKSESHTGHLLEHQR |
| | | | | THTGEKPYKCPECGKSFSTHLDLIRHQRTHTGEKPYKCPECG |
| | | | | KSFSQSSNLVRHQRTHTGEKPYKCPECGKSFSQSSSLVRHQR |
| | | | | THTGKKTS (SEQ ID NO: 870) |
| 5′ | ZnF42F | TGAATGAGTCCTGTCCATCTT (SEQ | 55.08 | LEPGEKPYKCPECGKSFSTTGALTEHQRTHTGEKPYKCPECG |
| | | ID NO: 863) | | KSFSTSGNLTEHQRTHTGEKPYKCPECGKSFSDPGALVRHQR |
| | | | | THTGEKPYKCPECGKSFSTKNSLTEHQRTHTGEKPYKCPECG |
| | | | | KSFSHRTTLINHQRTHTGEKPYKCPECGKSFSRRDELNVHQR |
| | | | | THTGEKPYKCPECGKSFSQAGHLASHQRTHTGKKTS (SEQ |
| | | | | ID NO: 871) |
| 5′ | ZnF43F | AAGATTAGAACAAATGTCCAG (SEQ | 60.20 | LEPGEKPYKCPECGKSFSRADNLTEHQRTHTGEKPYKCPECG |
| | | ID NO: 864) | | KSFSDPGALVRHQRTHTGEKPYKCPECGKSFSTTGNLTVHQR |
| | | | | THTGEKPYKCPECGKSFSSPADLTRHQRTHTGEKPYKCPECG |
| | | | | KSFSQLAHLRAHQRTHTGEKPYKCPECGKSFSHKNALQNHQR |
| | | | | THTGEKPYKCPECGKSFSRKDNLKNHQRTHTGKKTS (SEQ |
| | | | | ID NO: 872) |
| 3′ | ZnF44R | ACTCTAAGCAGCAATGTA (SEQ ID | 59.94 | LEPGEKPYKCPECGKSFSQSSSLVRHQRTHTGEKPYKCPECG |
| | | NO: 865) | | KSFSTTGNLTVHQRTHTGEKPYKCPECGKSESERSHLREHQR |
| | | | | THTGEKPYKCPECGKSFSERSHLREHQRTHTGEKPYKCPECG |
| | | | | KSFSQNSTLTEHQRTHTGEKPYKCPECGKSESTHLDLIRHQR |
| | | | | THTGKKTS (SEQ ID NO: 873) |
| 5′ | ZnF45R | TGGGATAGTGAAAATGTC (SEQ ID | 57.10 | LEPGEKPYKCPECGKSFSDPGALVRHQRTHTGEKPYKCPECG |
| | | NO: 866) | | KSFSTTGNLTVHQRTHTGEKPYKCPECGKSFSQSSNLVRHQR |
| | | | | THTGEKPYKCPECGKSFSHRTTLTNHQRTHTGEKPYKCPECG |
| | | | | KSFSTSGNLVRHQRTHTGEKPYKCPECGKSESRSDHLTTHQR |
| | | | | THTGKKTS (SEQ ID NO: 874) |
| 5′ | ZnF4 | AAAACTTGGGTCACTAAAATAGATGAT | 61.20 | LEPGEKPYKCPECGKSFSTSGNLVRHQRTHTGEKPYKCPECG |
| | 6R | (SEQ ID NO: 867) | | KSFSTSGNLVRHQRTHTGEKPYKCPECGKSFSQKSSLIAHQR |
| | | | | THTGEKPYKCPECGKSFSQRANLRAHQRTHTGEKPYKCPECG |
| | | | | KSFSTHLDLIRHQRTHTGEKPYKCPECGKSFSDPGALVRHQR |
| | | | | THTGEKPYKCPECGKSESRSDHLTTHQRTHTGEKPYKCPECG |
| | | | | KSFSTHLDLIRHQRTHTGEKPYKCPECGKSFSQRANLRAHQR |
| | | | | THTGKKTS (SEQ ID NO: 875) |
| 5′ | ZnF47R | AAACATGGAAAAGGTCAAAAACTTGGG | 43.59 | LEPGEKPYKCPECGKSFSRSDKLVRHQRTHTGEKPYKCPECG |
| | | (SEQ ID NO: 868) | | KSFSTTGALTEHQRTHTGEKPYKCPECGKSESQRANLRAHQR |
| | | | | THTGEKPYKCPECGKSFSQSGNLTEHQRTHTGEKPYKCPECG |
| | | | | KSFSTSGHLVRHQRTHTGEKPYKCPECGKSFSQRANLRAHQR |
| | | | | THTGEKPYKCPECGKSFSQRAHLERHQRTHTGEKPYKCPECG |
| | | | | KSFSTSGNLTEHQRTHTGEKPYKCPECGKSESQRANLRAHQR |
| | | | | THTGKKTS (SEQ ID NO: 876) |
| 3′ | ZnF48R | AATGACTAGAATGAAGTCCTACTG | 59.44 | LEPGEKPYKCPECGKSFSRNDALTEHQRTHTGEKPYKCPECG |
| | | (SEQ ID NO: 869) | | KSFSQNSTLTEHQRTHTGEKPYKCPECGKSESDPGALVRHQR |
| | | | | THTGEKPYKCPECGKSFSQSSNLVRHQRTHTGEKPYKCPECG |
| | | | | KSFSTTGNLTVHQRTHTGEKPYKCPECGKSESREDNLHTHQR |
| | | | | THTGEKPYKCPECGKSFSDPGNLVRHQRTHTGEKPYKCPECG |
| | | | | KSFSTTGNLTVHQRTHTGKKTS (SEQ ID NO: 877) |

No Sequences have Target site overlap (TSO). The first and last 4 amino acid residues may be omitted from the amino acid code.
Available on the world wide web at scripps. edu/barbas/zfdesign/searchsequence.php

In embodiments, the present disclosure relates to a system having nucleic acids encoding the enzyme (e.g., without limitation, the helper enzyme) and the donor DNA, respectively.

### Linkers

In some embodiments, the targeting element comprises a nucleic acid binding component of a gene-editing system. In some embodiments, the helper enzyme the targeting element are connected. Without wishing to be bound by a particular theory, the targeting element may refer to a nucleic acid binding component of the gene-editing system. In some embodiments, the helper enzyme and the targeting element are connected. For example, in embodiments, the the helper enzyme and the targeting element are fused to one another or linked via a linker to one another. In embodiments, the linker is a covalent or non-covalent linker. In embodiments, the linker comprises an amino acid sequence of AKLAGGAPAVGGGPKAADKFAATGGS (SEQ ID NO: 8), or a variant thereof having a substitution or deletion.

In some embodiments, the linker is a flexible linker. In some embodiments, the flexible linker is substantially comprised of glycine and serine residues, optionally wherein the flexible linker comprises (Gly₄Ser)ₙ, where n is an integer from 1 to 12. In some embodiments, the flexible linker is of about 20, or about 30, or about 40, or about 50, or about 60 amino acid residues. In embodiments, the flexible linker is about 50, or about 100, or about 150, or about 200 amino acid residues in length. In embodiments, the flexible linker comprises at least about 150 nucleotides (nt), or at least about 200 nt, or at least about 250 nt, or at least about 300 nt, or at least about 350 nt, or at least about 400 nt, or at least about 450 nt, or at least about 500 nt, or at least about 500 nt, or at least about 600 nt. In embodiments, the flexible linker comprises from about 450 nt to about 500 nt.

In embodiments, the linker is or comprises (GSS) 4 or the linker is GS flanked on either side of a DNA binding domain, optionally TALE and ZnF. In embodiments, the linker connects the targeting element to the N-terminus of the helper enzyme or connects the targeting element within the helper enzyme.

In embodiment, the linker is a non-covalent linker. In embodiments, the non-covalent linker comprises an epitope tag. In embodiments, the non-covalent linker comprises an ALFA tag. In embodiments, the ALFA tag further comprises recombinant variable domains of heavy-chain-only antibodies (VHH) (NANOBODY). In embodiments, the non-covalent linker comprises recombinant variable domains of heavy-chain-only antibodies (VHH) (NANOBODY). In embodiments, the non-covalent linker comprises recombinant variable domains of heavy-chain-only antibodies (VHH) (NANOBODY) binding to ALFA tagged protein (NbALFA). In embodiments, the linker comprises a monobody. In embodiments, the linker comprises an ALFA tag and monobody. In embodiments, the ALFA tag comprises an amino acid sequence of SRLEEELRRRLTE (SEQ ID NO: 892), or a variant thereof having a substitution or deletion.

In embodiments, the helper enzyme further comprises a linker inserted after a residue selected from S60-E70 relative to SEQ ID NO: 1. In embodiments, the helper enzyme further comprises a linker inserted after the G66 residue relative to SEQ ID NO: 1. In embodiments, the helper enzyme further comprises a linker inserted after the S60 residue relative to SEQ ID NO: 1. In embodiments, the helper enzyme further comprises a linker inserted after the E70 residue relative to SEQ ID NO: 1. In embodiments, the helper enzyme further comprises a linker inserted after the L80 residue relative to SEQ ID NO: 1. In embodiments, the helper enzyme further comprises a linker inserted after the N90 residue relative to SEQ ID NO: 1. In embodiments, the helper enzyme further comprises a linker inserted after the D100 residue relative to SEQ ID NO: 1.

In embodiments, the linker is inserted immediately after residue G66 relative to SEQ ID NO: 1.

### Inteins

Inteins (INTervening protEINS) are mobile genetic elements that are protein domains, found in nature, with the capability to carry out the process of protein splicing. See Sarmiento & Camarero (2019) Current protein & peptide science, 20(5), 408-424, which is incorporated by reference herein in its entirety. Protein spicing is a post-translation biochemical modification which results in the cleavage and formation of peptide bonds between precursor polypeptide segments flanking the intein. Id. Inteins apply standard enzymatic strategies to excise themselves post-translationally from a precursor protein via protein splicing. Nanda et al., Microorganisms vol. 8,12 2004. 16 Dec. 2020, doi:10.3390/microorganisms8122004. An intein can splice its flanking N- and C-terminal domains to become a mature protein and excise itself from a sequence. For example, split inteins have been used to control the delivery of heterologous genes into transgenic organisms. See Wood & Camarero (2014) J Biol Chem. 289(21):14512-14519. This approach relies on splitting the target protein into two segments, which are then post-translationally reconstituted in vivo by protein trans-splicing (PTS). See Aboye & Camarero (2012) J. Biol. Chem. 287, 27026-27032. More recently, an intein-mediated split-Cas9 system has been developed to incorporate Cas9 into cells and reconstitute nuclease activity efficiently. Truong et al., Nucleic Acids Res. 2015, 43(13), 6450-6458. The protein splicing excises the internal region of the precursor protein, which is then followed by the ligation of the N-extein and C-extein fragments, resulting in two polypeptides—the excised intein and the new polypeptide produced by joining the C- and N-exteins. Sarmiento & Camarero (2019).

In embodiments, intein-mediated incorporation of DNA binders such as, without limitation, dCas9, dCas12j, or TALEs, allows creation of a split-enzyme system such as, without limitation, split helper system, that permits reconstitution of the full-length enzyme, e.g., helper, from two smaller fragments. This allows avoiding the need to express DNA binders at the N- or C-terminus of an enzyme, e.g., helper. In this approach, the two portions of an enzyme, e.g., helper, are fused to the intein and, after co-expression, the intein allows producing a full-length enzyme, e.g., helper, by post-translation modification. Thus, in embodiments, a nucleic acid encoding the enzyme capable of targeted genomic integration by transposition comprises an intein. In embodiments, the nucleic acid encodes the helper enzyme in the form of first and second portions with the intein encoded between the first and second portions, such that the first and second portions are fused into a functional helper enzyme upon post-translational excision of the intein from the helper enzyme.

In embodiments, an intein is a suitable ligand-dependent intein, for example, an intein selected from those described in U.S. Pat. No. 9,200,045; Mootz et al., J. Am. Chem. Soc. 2002; 124, 9044-9045; Mootz et al., J. Am. Chem. Soc. 2003; 125, 10561-10569; Buskirk et al., Proc. Natl. Acad. Sci. USA. 2004; 101, 10505-10510; Skretas & Wood. Protein Sci. 2005; 14, 523-532; Schwartz, et al., Nat. Chem. Biol. 2007; 3, 50-54; Peck et al., Chem. Biol. 2011; 18(5), 619-630; the entire contents of each of which are hereby incorporated by reference herein.

In embodiments the intein is NpuN (Intein-N) (SEQ ID NO: 423) and/or NpuC (Intein-C) (SEQ ID NO: 424), or a variant thereof, e.g., a sequence having at least about 90%, or at least about 93%, or at least about 95%, or at least about 97%, or at least about 98%, or at least about 99% identity thereto.

SEQ ID NO: 423: nucleotide sequence of NpuN
(Intein-N)
GGCGGATCTGGCGGTAGTGCTGAGTATTGTCTGAGTTACGAAACGGAAA
TACTCACGGTTGAGTATGGGCTTCTTCCAATTGGCAAAATCGTTGAAAA
GCGCATAGAGTGTACGGTGTATTCCGTCGATAACAACGGTAATATCTAC
ACCCAGCCGGTAGCTCAGTGGCACGACCGAGGCGAACAGGAAGTGTTCG
AGTATTGCTTGGAAGATGGCTCCCTTATCCGCGCCACTAAAGACCATAA
GTTTATGACGGTTGACGGGCAGATGCTGCCTATAGACGAAATATTTGAG
AGAGAGCTGGACTTGATGAGAGTCGATAATCTGCCAAAT
SEQ ID NO: 424: nucleotide sequence of NpuC
(Intein-C)
GGCGGATCTGGCGGTAGTGGGGGTTCCGGATCCATAAAGATAGCTACTA
GGAAATATCTTGGCAAACAAAACGTCTATGACATAGGAGTTGAGCGAGA
TCACAATTTTGCTTTGAAGAATGGGTTCATCGCGTCTAATTGCTTCAAC
GCTAGCGGCGGGTCAGGAGGCTCTGGTGGAAGC

### Dimerization Enhancers

In embodiments, a nucleic acid encoding the helper enzyme capable of targeted genomic integration by transposition comprises a dimerization enhancer. In embodiments, the nucleic acid encodes the helper enzyme in the form of first and second portions with the dimerization enhancer encoded between the first and second portions, such that the first and second portions are fused into a functional helper enzyme upon post-translational excision of the dimerization enhancer from the helper enzyme. In embodiments, the dimerization enhancer is suitable for linking the helper enzyme and the targeting element. In embodiments, the dimerization enhancer is selected from: a protein comprising a SH3 domain, biotin, avidin, or a rapamycin binder, optionally, wherein the rapamycin binder is FKBP12 or mTOR, or a variant thereof.

### Nucleic Acids of the Disclosure

In embodiments, a nucleic acid encoding the enzyme (e.g., without limitation, the helper enzyme) is RNA. In embodiments, a nucleic acid encoding the transgene is DNA. In embodiments, thr RNA is or comprises messenger RNA (mRNA). In embodiments, the mRNA is or comprises modified mRNA (mmRNA).

In embodiments, the enzyme (e.g., without limitation, the helper enzyme) is encoded by a recombinant or synthetic nucleic acid. In embodiments, the nucleic acid is RNA, optionally a helper RNA. In embodiments, the nucleic acid is RNA that has a 5′-m7G cap (cap0, or cap1, or cap2), optionally with pseudouridine substitution (e.g., without limitation n-methyl-pseudouridine), and optionally a poly-A tail of about 30, or about 50, or about 100, of about 150 nucleotides in length. In embodiments, the poly-A tail is of about 30 nucleotides in length, optionally 34 nucleotides in length. In embodiments, a nuclear localization signal is placed before the enzyme start codon at the N-terminus, optionally at the C-terminus.

In embodiments, the nucleic acid that is RNA has a 5′-m7G cap (cap 0, or cap 1, or cap 2).

In embodiments, the nucleic acid comprises a 5′ cap structure, a 5′-UTR comprising a Kozak consensus sequence, a 5′-UTR comprising a sequence that increases RNA stability in vivo, a 3′-UTR comprising a sequence that increases RNA stability in vivo, and/or a 3′ poly(A) tail.

In embodiments, the enzyme (e.g., without limitation, a helper) is incorporated into a vector or a vector-like particle. In embodiments, the vector is a non-viral vector.

In embodiments, a nucleic acid encoding the helper enzyme in accordance with embodiments of the present disclosure, is DNA.

In various embodiments, a construct comprising a donor is any suitable genetic construct, such as a nucleic acid construct, a plasmid, or a vector. In various embodiments, the construct is DNA, which is referred to herein as a donor DNA. In embodiments, sequences of a nucleic acid encoding the donor is codon optimized to provide improved mRNA stability and protein expression in mammalian systems.

In embodiments, the helper enzyme and the donor are included in different vectors. In embodiments, the helper enzyme and the donor are included in the same vector.

In various embodiments, a nucleic acid encoding the helper enzyme capable of targeted genomic integration by transposition (e.g., without limitation, the helper enzyme) is RNA (e.g., helper RNA), and a nucleic acid encoding a donor is DNA.

In embodiments, the one or more nucleic acid-based agents is in the form of DNA. In embodiments, the DNA is or comprises plasmid DNA. In embodiments, the plasmid DNA has a size of up to about 10 kb, or up to about 12 kb, up to about 15 kb, or about 7.5 to about 15 kb, or about 10 to about 15 kb.

In embodiments, the plasmid is conjugative, non-conjugative or mobilized. In embodiments, the plasmid is selected from fertility F-plasmids, resistance plasmids, Col plasmids, degradative plasmids, or virulence plasmids. In embodiments, the plasmid is an artificially construct plasmid that is used as vectors in genetic engineering. In embodiments, the plasmid is selected from miniplasmid, nanoplasmid, doggybone or close-ended linear DNA.

In embodiments, the composition further comprises a donor nucleic acid or is suitable for insertion of a donor nucleic acid, optionally wherein the donor nucleic acid is a transposon. In embodiments, the helper enzyme is suitable of inserting a donor nucleic acid comprising a transgene in a genomic safe harbor site (GSHS) and/or wherein the targeting element is suitable for directing the helper enzyme to a GSHS. In embodiments, a donor DNA and a helper RNA are suitable for transfection at a donor DNA to helper RNA ratio of about 1 to about 4, or about 1 to about 2, or about 1 to about 1. In embodiments, the composition further comprises a nucleic acid encoding a donor comprising a transgene to be integrated, optionally wherein the transgene is defective or substantially absent in a disease state. In embodiments, the transgene comprises a cargo nucleic acid sequence and a first and a second donor end sequences. In embodiments, transgene comprises a cargo nucleic acid sequence and a first and a second donor end sequences.

As would be appreciated in the art, a donor often includes an open reading frame that encodes a transgene at the middle of donor and terminal repeat sequences at the 5′ and 3′ end of the donor. The translated helper (e.g., without limitation, the helper enzyme) binds to the 5′ and 3′ sequence of the donor and carries out the transposition function.

In embodiments, a donor is used interchangeably with transposable elements, which are used to refer to polynucleotides capable of inserting copies of themselves into other polynucleotides. The term donor is well known to those skilled in the art and includes classes of donors that can be distinguished on the basis of sequence organization, for example inverted terminal sequences at each end, and/or directly repeated long terminal repeats (LTRs) at the ends. In embodiments, the donor as described herein may be described as a piggyBac like element, e.g., a donor element that is characterized by its traceless excision, which recognizes TTAA (SEQ ID NO: 440) sequence and restores the sequence at the insert site back to the original TTAA (SEQ ID NO: 440) sequence after removal of the donor.

In embodiments, the donor is flanked by one or more end sequences or terminal ends. In embodiments, the donor is or comprises a gene encoding a complete polypeptide. In embodiments, the donor is or comprises a gene which is defective or substantially absent in a disease state.

In embodiments, a transgene is associated with various regulatory elements that are selected to ensure stable expression of a construct with the transgene. Thus, in embodiments, a transgene is encoded by a non-viral vector (e.g., without limitation, a DNA plasmid) that can comprise one or more insulator sequences that prevent or mitigate activation or inactivation of nearby genes. The insulators flank the donor (transgene cassette) to reduce transcriptional silencing and position effects imparted by chromosomal sequences. As an additional effect, the insulators can eliminate functional interactions of the transgene enhancer and promoter sequences with neighboring chromosomal sequences. In embodiments, the one or more insulator sequences comprise an HS4 insulator (1.2-kb 5′-HS4 chicken β-globin (cHS4) insulator element) and an D4Z4 insulator (tandem macrosatellite repeats linked to Facio-Scapulo-Humeral Dystrophy (FSHD). In embodiments, the sequences of the HS4 insulator and the D4Z4 insulator are as described in Rival-Gervier et al. Mol Ther. 2013 August; 21(8):1536-50, which is incorporated herein by reference in its entirety.

In embodiments, the transgene is inserted into a GSHS location in a host genome. GSHSs is defined as loci well-suited for gene transfer, as integrations within these sites are not associated with adverse effects such as proto-oncogene activation, tumor suppressor inactivation, or insertional mutagenesis. GSHSs can defined by the following criteria: (1) distance of at least 50 kb from the 5′ end of any gene, (2) distance of at least 300 kb from any cancer-related gene, (3) distance of at least 300 kb from any microRNA (miRNA), (4) location outside a transcription unit, and (5) location outside ultra-conserved regions (UCRs) of the human genome. See Papapetrou et al. Nat Biotechnol 2011; 29:73-8; Bejerano et al. Science 2004; 304:1321-5.

Furthermore, the use of GSHS locations can allow stable transgene expression across multiple cell types. One such site, chemokine C—C motif receptor 5 (CCR5) has been identified and used for integrative gene transfer. CCR5 is a member of the beta chemokine receptor family and is required for the entry of R5 tropic viral strains involved in primary infections. A homozygous 32 bp deletion in the CCR5 gene confers resistance to HIV-1 virus infections in humans. Disrupted CCR5 expression, naturally occurring in about 1% of the Caucasian population, does not appear to result in any reduction in immunity. Lobritz at al., Viruses 2010; 2:1069-105. A clinical trial has demonstrated safety and efficacy of disrupting CCR5 via targetable nucleases. Tebas at al., HIV. N Engl J Med 2014; 370:901-10.

In embodiments, the donor is under control of a tissue-specific promoter. The tissue-specific promoter is, e.g., without limitation, a liver-specific promoter. In embodiments, the liver-specific promoter is an LP1 promoter that, in embodiments, is a human LP1 promoter. The LP1 promoter is described, e.g., in Nathwani et al. Blood vol. 2006; 107(7):2653-61, and it is constructed, without limitation, as described in Nathawani et al.

It should be appreciated however that a variety of promoters can be used, including other tissue-specific promoters, inducible promoters, constitutive promoters, etc.

In embodiments, the present nucleic acids include polymeric form of nucleotides of any length, either ribonucleotides or deoxyribonucleotides, or analogs or derivatives thereof. In embodiments, there is provided double- and single-stranded DNA, as well as double- and single-stranded RNA, and RNA-DNA hybrids. In embodiments, transcriptionally-activated polynucleotides such as methylated or capped polynucleotides are provided. In embodiments, the present compositions are mRNA or DNA.

In embodiments, the present non-viral vectors are linear or circular DNA molecules that comprise a polynucleotide encoding a polypeptide and is operably linked to control sequences, wherein the control sequences provide for expression of the polynucleotide encoding the polypeptide. In embodiments, the non-viral vector comprises a promoter sequence, and transcriptional and translational stop signal sequences. Such vectors may include, among others, chromosomal and episomal vectors, e.g., vectors bacterial plasmids, from donors, from yeast episomes, from insertion elements, from yeast chromosomal elements, and vectors from combinations thereof. The present constructs may contain control regions that regulate as well as engender expression.

In embodiments, the construct comprising the helper enzyme and/or transgene is codon optimized. Transgene codon optimization is used to optimize therapeutic potential of the transgene and its expression in the host organism. Codon optimization is performed to match the codon usage in the transgene with the abundance of transfer RNA (tRNA) for each codon in a host organism or cell. Codon optimization methods are known in the art and described in, for example, WO 2007/142954, which is incorporated by reference herein in its entirety. Optimization strategies can include, for example, the modification of translation initiation regions, alteration of mRNA structural elements, and the use of different codon biases.

In embodiments, the construct comprising the helper enzyme and/or transgene includes several other regulatory elements that are selected to ensure stable expression of the construct. Thus, in embodiments, the non-viral vector is a DNA plasmid that can comprise one or more insulator sequences that prevent or mitigate activation or inactivation of nearby genes. In embodiments, the one or more insulator sequences comprise an HS4 insulator (1.2-kb 5′-HS4 chicken β-globin (cHS4) insulator element) and an D4Z4 insulator (tandem macrosatellite repeats linked to Facio-Scapulo-Humeral Dystrophy (FSHD). In embodiments, the sequences of the HS4 insulator and the D4Z4 insulator are as described in Rival-Gervier et al. Mol Ther. 2013 August; 21(8):1536-50, which is incorporated herein by reference in its entirety. In embodiments, the gene of the construct comprising the helper enzyme and/or transgene is capable of transposition in the presence of a helper. In embodiments, the non-viral vector in accordance with embodiments of the present disclosure comprises a nucleic acid construct encoding a helper. The helper (e.g., without limitation, the helper enzyme of the present disclosure) is an RNA helper plasmid. In embodiments, the non-viral vector further comprises a nucleic acid construct encoding a DNA helper plasmid. In embodiments, the helper is an in vitro-transcribed mRNA helper. The helper (e.g., without limitation, the helper enzyme of the present disclosure) is capable of excising and/or transposing the gene from the construct comprising the helper enzyme and/or transgene to site- or locus-specific genomic regions.

In embodiments, the enzyme (e.g., without limitation, the helper enzyme) and the donor are included in the same vector.

In embodiments, the helper enzyme is disposed on the same (cis) or different vector (trans) than a donor with a transgene. Accordingly, in embodiments, the helper enzyme and the donor encompassing a transgene are in cis configuration such that they are included in the same vector. In embodiments, the helper enzyme and the donor encompassing a transgene are in trans configuration such that they are included in different vectors. The vector is any non-viral vector in accordance with the present disclosure.

In some aspects, a nucleic acid encoding the donor system of the present disclosure capable of targeted genomic integration by transposition (e.g., a helper) in accordance with embodiments of the present disclosure is provided. The nucleic acid is or comprises DNA or RNA. In embodiments, the nucleic acid encoding the helper enzyme is DNA. In embodiments, the nucleic acid encoding the helper enzyme capable of targeted genomic integration by transposition (e.g., a helper of the present disclosure) is RNA such as, e.g., helper RNA. In embodiments, the helper is incorporated into a vector. In embodiments, the vector is a non-viral vector.

In embodiments, the enzyme or variant thereof is incorporated into a vector or a vector-like particle. In embodiments, the vector or a vector-like particle comprises one or more expression cassettes. In embodiments, the vector or a vector-like particle comprises one expression cassette. In embodiments, the expression cassette further comprises the enzyme or variant thereof, the transgene, the donor end sequences, or a combination thereof. In embodiments, the enzyme or variant thereof, the transgene, the donor end sequences, or a combination thereof are incorporated into one or more vectors or vector-like particles. In embodiments, the enzyme or variant thereof, the transgene, the donor end sequences, or combination thereof are incorporated into a same vector or vector-like particle. In embodiments, the enzyme or variant thereof, the transgene, the donor end sequences, or combination thereof is incorporated into different vectors or vector-like particles. In embodiments, the vector or vector-like particle is nonviral.

In embodiments, the donor is under the control of at least one tissue-specific promoter. In embodiments, at least one tissue-specific promoter is a single promoter. In embodiments, at least one tissue-specific promoter is under the control of a dual promoter or a tandem promoter. In embodiments, the transgene to be integrated comprises at least one gene of interest. In embodiments, the transgene to be integrated comprises one gene of interest. In embodiments, the transgene to be integrated comprises two or more genes of interest.

In embodiments, the present helper enzyme can be in the form or an RNA or DNA and have one or two N-terminus nuclear localization signal (NLS) to shuttle the protein more efficiently into the nucleus. For example, in embodiments, the present helper enzyme further comprises one, two, three, four, five, or more NLSs. Examples of NLS are provided in Kosugi et al. (J. Biol. Chem. (2009) 284:478-485; incorporated by reference herein). In a particular embodiment, the NLS comprises the consensus sequence K(K/R)X(K/R) (SEQ ID NO: 348). In an embodiment, the NLS comprises the consensus sequence (K/R)(K/R)X₁₀₋₁₂(K/R)_{3/5} (SEQ ID NO: 349), where (K/R)_{3/5} represents at least three of the five amino acids is either lysine or arginine. In an embodiment, the NLS comprises the c-myc NLS. In a particular embodiment, the c-myc NLS comprises the sequence PAAKRVKLD (SEQ ID NO: 350). In a particular embodiment, the NLS is the nucleoplasmin NLS. In embodiments, the nucleoplasmin NLS comprises the sequence KRPAATKKAGQAKKKK (SEQ ID NO: 351). In embodiments, the NLS comprises the SV40 Large T-antigen NLS. In embodiments, the SV40 Large T-antigen NLS comprises the sequence PKKKRKV (SEQ ID NO: 352). In a particular embodiment, the NLS comprises three SV40 Large T-antigen NLSs (e.g., DPKKKRKVDPKKKRKVDPKKKRKV (SEQ ID NO: 353). In embodiments, the NLS may comprise mutations/variations in the above sequences such that they contain 1 or more substitutions, additions, or deletions (e.g., about 1, or about 2, or about 3, or about 4, or about 5, or about 10 substitutions, additions, or deletions).

In some aspects, a host cell comprising the nucleic acid in accordance with embodiments of the present disclosure is provided.

### Lipids and LNP Delivery

In embodiments, a composition or a nucleic acid in accordance with embodiments of the present disclosure is provided wherein the composition is in the form of a lipid nanoparticle (LNP). In embodiments, the composition is encapsulated in an LNP.

In embodiments, a nucleic acid encoding the helper enzyme and a nucleic acid encoding the transgene are contained within the same lipid nanoparticle (LNP). In embodiments, the nucleic acid encoding the helper enzyme and the nucleic acid encoding the donor are a mixture incorporated into or associated with the same LNP. In embodiments, the polynucleotide encoding the helper enzyme and the polynucleotide encoding the donor are in the form of the same LNP, optionally in a co-formulation.

In embodiments, the LNP comprises one or more lipids selected from 1,2-dioleoyl-3-trimethylammonium propane (DOTAP), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy (polyethylene glycol)-2000] (DSPE-PEG), 1,2-dimyristoyl-rac-glycero-3-methoxypolyethyleneglycol-2000 (DMG-PEG 2K), and 1,2 distearol-sn-glycerol-3phosphocholine (DSPC) and/or comprising of one or more molecules selected from polyethylenimine (PEI) and poly(lactic-co-glycolic acid) (PLGA), and N-Acetylgalactosamine (GalNAc).

In embodiments, an LNP is as described, e.g., in Patel et al., J Control Release 2019; 303:91-100. The LNP can comprise one or more of a structural lipid (e.g., DSPC), a PEG-conjugated lipid (CDM-PEG), a cationic lipid (MC3), cholesterol, and a targeting ligand (e.g., GalNAc).

In embodiments, the one or more lipids comprise cationic lipids; anionic lipids; neutral lipids; multi-valent charged lipids; and zwitterionic lipids.

In embodiments, the one or more lipids include a delivery lipid. In embodiments, the delivery lipid is or comprises a cationic lipid. In embodiments, the cationic lipid is ionizable and/or hydrolysable. In embodiments, the delivery lipid encapsulates, in whole or in part, the one or more nucleic acid-based agents. In embodiments, the delivery lipid, e.g., when contacted with a cell, promotes or increases endosomal escape. In embodiments, the delivery lipid is or comprises MC3 (DLin-MC3-DMA), C12-200 (1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol)), SM102 (9-Heptadecanyl 8-{(2-hydroxyethyl)[6-oxo-6-(undecyloxy) hexyl]amino}octanoate), squaramide (3,4-diaminocyclobut-3-ene-1,2-dione; SQAM), SS-EC (ssPalmE-P4C2), SS-OP (ssPalmO-Phe), [(4-hydroxybutyl)azanediyl]di(hexane-6,1-diyl)bis(2-hexyldecanoate), KC2 (DLin-KC2-DMA), DOTAP (Dioleoyl-3-trimethylammonium propane), cKK-E12 (3,6-bis({4-[bis(2-hydroxydodecyl)amino]butyl})piperazine-2,5-dione), ATX-001 (di((Z)-non-2-en-1-yl) 8,8′-((2-((2-(dimethylamino)ethyl)thio)acetyl)azanediyl)dioctanoate), ATX-002 (di((Z)-non-2-en-1-yl) 8,8′-((((2-(dimethylamino)ethyl)thio)carbonyl)azanediyl)dioctanoate), ATX-012, ATX-61, ATX-0081, ATX-0095, Lipid 5 (8-[(2-hydroxyethyl)[8-(nonyloxy)-8-oxooctyl]amino]-octanoic acid, 1-octylnonyl ester), and/or ALC-0315 (2-hexyl-decanoic acid, 1,1′-[[(4-hydroxybutyl)imino]di-6,1-hexanediyl] ester, [(4-Hydroxybutyl)azanediyl]di(hexane-6,1-diyl)bis(2-hexyldecanoate)). Various ATX lipids that are in embodiments delivery lipids of the disclosure are described in WO2018222890 and WO2015074085, each of which is hearby incorporate by reference in its entirety. In embodiments, the delivery lipid is or comprises DLin-DMA, DLin-K-DMA, DLin-KC2-DMA, DLin-MC3-DMA, 98N12-5, and C12-200. In embodiments, the one or more lipids include one type of delivery lipid. In embodiments, the one or more lipids include more than one type of delivery lipid. In embodiments, the one or more lipids include two types of delivery lipid. In embodiments, the one or more lipids include both MC3 and C12-200.

In embodiments, the one or more lipids include cholesterol. In embodiments, the cholesterol provides increased or enhanced LNP stability.

In embodiments, the one or more lipids include a helper lipid. In embodiments, the helper lipid encapsulates, in whole or in part, the one or more nucleic acid-based agents. In embodiments, the helper lipid, e.g., when contacted with a cell, promotes or increases endosomal escape. In embodiments, the helper lipid is a phospholipid. In embodiments the helper lipid is a neutral lipid. In embodiments, the neutral lipid is or comprises dioleoylphosphatidylethanolamine (DOPE) and/or 1,2-Dioleoyl-sn-glycero-3-phosphocholine (DOPC). In embodiments, the helper lipid is a cationic lipid. In embodiments, the cationic lipid is or comprises N-[1-(2,3-dioleoyloxy) propyl]-N,N,N-trimethylammonium chloride (DOTMA), 1,2-bis(oleoyloxy)-3-3-(trimethylammonia) propane (DOTAP), or 1,2-dioleoyl-3-dimethylammonium-propane (DODAP). In embodiments, the helper lipid is or comprises DOPE, DOPC, and/or Distearoylphosphatidylcholine (DSPC). In embodiments, the helper lipid is or comprises both DOPE and DSPC. In embodiments, the helper lipid is or comprises both DOPE and DOPC. In embodiments, the helper lipid is or comprises all of DOPE, DSPC, and DOPC.

In embodiments, the one or more lipids include a PEGylated lipid. In embodiments, the PEGylated lipid shields the LNP and reduces or prevents degradation of the LNP, e.g., in the bloodstream. In embodiments, the PEGylated lipid has a PEG molecule covalently attached to it, where the PEG has an average molecular weight of from about 1 kDa to about 50 kDa, or about 1 kDa to about 30 kDa, or from about 1 kDa to about 10 kDa, or about 1 kDa, or about 2 kDa, or about 3 kDa, or about 4 kDa, or about 5 kDa, or about 10 kDa. In embodiments, the PEG is a linear, a branched PEG, a star PEG, or a comb PEG. In embodiments the PEG is selected from PEG200, PEG300, PEG400, PEG600, PEG800, PEG1000, PEG1500, PEG2000, PEG3000, and PEG4000. In embodiments, the PEG is PEG2000. In embodiments the PEGylated lipid is or comprises 1,2-dimyristoyl-rac-glycero-3-methoxypolyethylene glycol-2000 (DMG-PEG). In embodiments, the DMG-PEG is or comprises 1,2-dimyristoyl-rac-glycero-3-methoxypolyethylene glycol-2000 (PEG-2k-DMG) and/or a PEG conjugated to a ceramide, e.g., C8 PEG 2k Ceramide, C16 PEG 2k Ceramide. In embodiments the PEGylated lipid is or comprises PEG-dimyristyloxypropyl (PEG-DMA), a PEG-distearyloxypropyl (PEG-DSA), PEG-diacylglycerol (PEG-DAG), or PEG-dialkyloxypropyl (PEG-DAA).

In embodiments, the one or more lipids do not include a PEGylated lipid. In embodiments, the one or more lipids do not include a PEGylated lipid, e.g., when being formulated for local administration.

In embodiments, the composition comprises a mixture of PEGylated lipids and free PEG chains.

In embodiments, a nanoparticle is a particle having a diameter of less than about 1000 nm. In embodiments, nanoparticles of the present disclosure have a greatest dimension (e.g., diameter) of about 500 nm or less, or about 400 nm or less, or about 300 nm or less, or about 200 nm or less, or about 100 nm or less. In embodiments, nanoparticles of the present disclosure have a greatest dimension ranging between about 50 nm and about 150 nm, or between about 70 nm and about 130 nm, or between about 80 nm and about 120 nm, or between about 90 nm and about 110 nm. In embodiments, the nanoparticles of the present disclosure have a greatest dimension (e.g., a diameter) of about 100 nm.

In some aspects, the cell in accordance with the present disclosure is prepared via an in vivo genetic modification method. In embodiments, a genetic modification in accordance with the present disclosure is performed via an ex vivo method.

In some aspects, the cell in accordance with the present disclosure is prepared by contacting a cell with a helper enzyme capable of targeted genomic integration by transposition (e.g., without limitation, the helper enzyme) in vivo. In embodiments, the cell is contacted with the helper enzyme ex vivo.

In embodiments, the present method provides high specific targeting as compared to a method that does not use the helper enzyme with a target selector.

### Therapeutic Applications

In embodiments, the transgene of interest in accordance with embodiments of the present disclosure can encode various genes.

In embodiments, the helper enzyme and the donor are included in the same pharmaceutical composition.

In embodiments, the helper enzyme and the donor are included in different pharmaceutical compositions.

In embodiments, the helper enzyme and the donor are co-transfected.

In embodiments the helper enzyme and the donor are transfected separately.

In embodiments, a transfected cell for gene therapy is provided, wherein the transfected cell is generated using the helper enzyme in accordance with embodiments of the present disclosure.

In embodiments, a method of delivering a cell therapy is provided, comprising administering to a patient in need thereof the transfected cell generated using the helper enzyme in accordance with embodiments of the present disclosure.

In embodiments, a method of treating a disease or condition using a cell therapy, comprising administering to a patient in need thereof the transfected cell generated using the helper enzyme in accordance with embodiments of the present disclosure.

In embodiments, there is provided a method for inserting a gene into the genome of a cell, comprising contacting a cell with the composition of the present disclosure.

In embodiments, there is provided a method for treating a disease or disorder ex vivo, comprising contacting a cell with the composition of the present disclosure and administering the cell to a subject in need thereof.

In embodiments, there is provided a method for treating a disease or disorder in vivo, comprising administering the composition of the present disclosure to a subject in need thereof.

In embodiments, the disease or condition may comprise cancer. In embodiments, the cancer is or comprises an adrenal cancer, a biliary track cancer, a bladder cancer, a bone/bone marrow cancer, a brain cancer, a breast cancer, a cervical cancer, a colorectal cancer, a cancer of the esophagus, a gastric cancer, a head/neck cancer, a hepatobiliary cancer, a kidney cancer, a liver cancer, a lung cancer, an ovarian cancer, a pancreatic cancer, a pelvis cancer, a pleura cancer, a prostate cancer, a renal cancer, a skin cancer, a stomach cancer, a testis cancer, a thymus cancer, a thyroid cancer, a uterine cancer, a lymphoma, a melanoma, a multiple myeloma, or a leukemia.

In embodiments, the cancer is selected from one or more of the basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; glioblastoma; hepatic carcinoma; hepatoma; intra-epithelial neoplasm; kidney or renal cancer; larynx cancer; leukemia; liver cancer; lung cancer; melanoma; myeloma; neuroblastoma; oral cavity cancer; ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; Hodgkin's lymphoma; non-Hodgkin's lymphoma; B-cell lymphoma; small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's Macroglobulinemia; chronic lymphocytic leukemia (CLL); acute lymphoblastic leukemia (ALL); and Hairy cell leukemia.

In embodiments, the cancer is selected from one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulvar cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), and Meigs syndrome.

In embodiments, the disease or condition is or comprises an infectious disease. In embodiments, the infectious disease is a coronavirus infection, optionally selected from infection with SAR-CoV, MERS-CoV, and SARS-CoV-2, or variants thereof.

In embodiments, the infectious disease is or comprises a disease comprising a viral infection, a parasitic infection, or a bacterial infection. In embodiments, the viral infection is caused by a virus of family Flaviviridae, a virus of family Picornaviridae, a virus of family Orthomyxoviridae, a virus of family Coronaviridae, a virus of family Retroviridae, a virus of family Paramyxoviridae, a virus of family Bunyaviridae, or a virus of family Reoviridae.

In embodiments, the virus of family Coronaviridae comprises a betacoronavirus or an alphacoronavirus, optionally wherein the betacoronavirus is selected from SARS-CoV-2, SARS-CoV, MERS-CoV, HCoV-HKU1, and HCoV-OC43, or the alphacoronavirus is selected from a HCoV-NL63 and HCoV-229E. In embodiments, the infectious disease comprises a coronavirus infection 2019 (COVID-19).

In embodiments, the method requires a single administration. In embodiments, the method requires a plurality of administrations.

### Isolated Cell

In some aspects of the present disclosure, an isolated cell is provided that comprises the transfected cell in accordance with embodiments of the present disclosure.

In some aspects, the present disclosure provides an ex vivo gene therapy approach. Accordingly, in embodiments, the method that is used to treat an inherited or acquired disease in a patient in need thereof comprises (a) contacting a cell obtained from a patient (autologous) or another individual (allogeneic) with a transfected cell in accordance with embodiments of the present disclosure; and (b) administering the cell to a patient in need thereof.

One of the advantages of ex vivo gene therapy is the ability to “sample” the transduced cells before patient administration. This facilitates efficacy and allows performing safety checks before introducing the cell(s) to the patient. For example, the transduction efficiency and/or the clonality of integration can be assessed before infusion of the product. The present disclosure provides transfected cells and methods that can be effectively used for ex vivo gene modification.

In embodiments, a composition comprising transfected cells in accordance with the present disclosure comprises a pharmaceutically acceptable carrier, excipient, or diluent.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile, and the fluid should be easy to draw up by a syringe. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds can be prepared with carriers that will protect the therapeutic compounds against rapid elimination from the body, such as a controlled release formulation, including implants and microencapsulated delivery systems. Biodegradable, biocompatible polymers can be used, such as collagen, ethylene vinyl acetate, polyanhydrides (e.g., poly[1,3-bis(carboxyphenoxy) propane-co-sebacic-acid] (PCPP-SA) matrix, fatty acid dimer-sebacic acid (FAD-SA) copolymer, poly (lactide-co-glycolide)), polyglycolic acid, collagen, polyorthoesters, polyethyleneglycol-coated liposomes, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811. Semisolid, gelling, soft-gel, or other formulations (including controlled release) can be used, e.g., when administration to a surgical site is desired. Methods of making such formulations are known in the art and can include the use of biodegradable, biocompatible polymers. See, e.g., Sawyer et al., Yale J Biol Med. 2006; 79(3-4): 141-152.

In embodiments, there is provided a method of transforming a cell using the construct comprising the helper enzyme and/or transgene described herein in the presence of a helper (e.g., without limitation, the helper enzyme) to produce a stably transfected cell which results from the stable integration of a gene of interest into the cell. In embodiments, the stable integration comprises an introduction of a polynucleotide into a chromosome or mini-chromosome of the cell and, therefore, becomes a relatively permanent part of the cellular genome.

In embodiments, there is provided a transgenic organism that may comprise cells which have been transformed by the methods of the present disclosure. In embodiments, the organism may be a mammal or an insect. When the organism is a mammal, the organism may include, but is not limited to, a mouse, a rat, a chimpanzee, an elephant, a dog, a rabbit, a raccoon, and the like. When the organism is an insect, the organism may include, but is not limited to, a fruit fly, an ant, a mosquito, a bollworm, and the like.

### Methods for Identifying Site-Specific Targeting to a Nucleic Acid

In aspects, there is provided a method for identifying site-specific targeting to a nucleic acid by a helper enzyme and a targeting element, comprising: (a) transfecting a cell with a donor plasmid, the helper enzyme and a targeting element, and a reporter plasmid, wherein: the donor plasmid comprises a first fragment of a reporter gene under the control of a promoter and a splice-donor site (SD); the reporter plasmid comprises a landing pad for the targeting element comprising site specific DNA binding recognition sites flanking a TTAA followed by a splice acceptor site (SA) and a second fragment of a reporter gene; and (b) splicing and integrating into the landing pad, to permit the reconstitution of the reporter gene from the fragments thereof and thereby causing a reporter readout. In embodiments, the method further comprises (c) amplifying the donor plasmid to identify targeting. In embodiments, the method further comprises (d) sequencing the amplified product to analyze integration in specific sequence regions. In embodiments, the SA and SD are spliced out of the donor plasmid in step (b).

In embodiments, the amplifying is via PCR. In embodiments, the sequencing is amplicon sequencing in embodiments, the fluorescent protein is or comprises a monomeric red fluorescent protein (mRFP). In embodiments, the mRFP is selected from mCherry, DsRed, mRFP1, mStrawberry, mOrange, and dTomato. In embodiments, the fluorescent protein is or comprises a green fluorescent protein (GFP). In embodiments, the reporter readout is fluorescence. In embodiments, the promoter is selected from cytomegalovirus (CMV), CMV enhancer fused to the chicken β-actin (CAG), chicken β-actin (CBA), simian vacuolating virus 40 (SV40), β glucuronidase (GUSB), polyubiquitin C gene (UBC), elongation-factor 1a subunit (EF-1α), and phosphoglycerate kinase (PGK).

In embodiments, the helper enzyme is a recombinase, integrase or a transposase. In embodiments, the helper enzyme is a mammal-derived transposase. In embodiments, the helper enzyme is derived from Bombyx mori, Xenopus tropicalis, Trichoplusia ni, Myotis lucifugus, Rhinolophus ferrumequinum, Rousettus aegyptiacus, Phyllostomus discolor, Myotis myotis, Pteropus vampyrus, Pipistrellus kuhlii, troglodytes, Molossus molossus, or Homo sapiens.

In embodiments, the composition comprises a helper enzyme or a nucleic acid encoding the helper enzyme, an amino acid sequence having at least about 80% sequence identity to SEQ ID NO: 1 and a non-polar aliphatic amino acid at position 2 of SEQ ID NO: 1, or a position corresponding thereto, one or more amino acid substitutions selected from S8X, C13X, and D416X of SEQ ID NO: 1 or a substitution at a position corresponding thereto, and one or more amino acid substitutions selected from Y281X, C282X, G283X, E284X, G285X, K286X, R287X, L288X, L289X, E290X, T291X, I332X, R333X, K334X, N335X, R336X, G337X, I338X, and P339X of SEQ ID NO: 1 or a substitution at a position corresponding thereto.

In embodiments, the composition comprises a helper enzyme or a nucleic acid encoding the helper enzyme and a targeting element or a nucleic acid encoding the targeting element wherein, the helper enzyme comprises an amino acid sequence having at least about 80% sequence identity to SEQ ID NO: 1 and has a non-polar aliphatic amino acid at position 2 of SEQ ID NO: 1, or a position corresponding thereto, one or more amino acid substitutions selected from S8X, C13X, and D416X of SEQ ID NO: 1 or a substitution at a position corresponding thereto, and one or more amino acid substitutions selected from Y281X, C282X, G283X, E284X, G285X, K286X, R287X, L288X, L289X, E290X, T291X, I332X, R333X, K334X, N335X, R336X, G337X, I338X, and P339X of SEQ ID NO: 1 or a substitution at a position corresponding thereto.

In embodiments, targeting element is or comprises one or more of a Cas enzyme, which is optionally catalytically inactive and which is optionally associated with a guide RNA (gRNA), a transcription activator-like effector (TALE) DNA binding domain (DBD), a Zinc finger (ZnF), a catalytically inactive transcription factor, catalytically inactive nickase, a transcriptional activator, a transcriptional repressor, a recombinase, a DNA methyltransferase, a histone methyltransferase, a paternally expressed gene 10 (PEG10), and a transposon-encoded polypeptide D (TnsD) or a variant thereof.

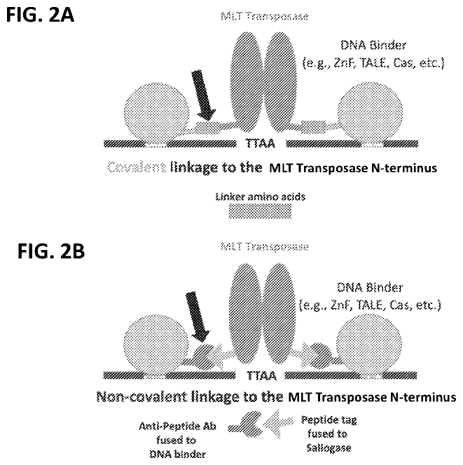

In embodiments, the method is substantially as in FIGS. 2A-2B, 4A-4C, and 7.

### Method of Designing a Transposase

In embodiments, there is provided a method of designing a transposase for binding identical target sites upstream and downstream of a target nucleic acid site, comprising: a) obtaining the transposase, the transposase being modified to have reduced or ablated inherent nucleic acid binding ability and having at least about 90%, or at least about 93%, at least about 95%, at least about 97%, at least about 98%, at least about 99% identity to the amino acid sequence of SEQ ID NO: 1, b) fusing or associating the transposase with at least two targeting elements, each targeting element being fused at or immediately after G66 relative to SEQ ID NO: 1, optionally wherein the fusing or associating the transposase with at least two targeting elements is by way of a linker, wherein the target nucleic acid site is a genomic safe harbor site of or comprising TTAA.

In embodiments, the linker is a covalent linker or a non-covalent linker. In embodiments, the linker comprises less than about 25 amino acids or 75 nucleotides. In embodiments, the linker comprises about 10 amino acids to about 20 amino acids or about 12 amino acids to about 15 amino acids, or about 30 nucleotides to about 60 nucleotides or about 36 nucleotides to about 45 nucleotides. In embodiments, the linker is substantially comprised of glycine (G) and serine(S) residues. In embodiments, the linker is or comprises (GSS)₄ or the linker is GS flanked on either side of a targeting element, optionally TALE and ZnF.

In embodiments, the linker comprises an amino acid sequence of AKLAGGAPAVGGGPKAADKFAATGGS (SEQ ID NO: 8) or a variant thereof having a substitution or deletion. In embodiments, the linker connects the targeting element to the N-terminus of the helper enzyme or connects the targeting element within the helper enzyme.

In embodiments, the linker is a non-covalent linker. In embodiments, the non-covalent linker comprises an epitope tag, optionally wherein the epitope tag comprises an ALFA tag. In embodiments, the ALFA tag further comprises recombinant variable domains of heavy-chain-only antibodies (VHH) (NANOBODY). In embodiments, the targeting elements are oriented on opposite sides of the transposase.

### Definitions

The following definitions are used in connection with the disclosure disclosed herein. Unless defined otherwise, all technical and scientific terms used herein have the same meaning as commonly understood to one of skill in the art to which this disclosure belongs.

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

An “effective amount,” when used in connection with medical uses is an amount that is effective for providing a measurable treatment, prevention, or reduction in the rate of pathogenesis of a disease of interest.

The term “in vivo” refers to an event that takes place in a subject's body.

The term “ex vivo” refers to an event which involves treating or performing a procedure on a cell, tissue and/or organ which has been removed from a subject's body. Aptly, the cell, tissue and/or organ may be returned to the subject's body in a method of treatment or surgery.

As used herein, the term “variant” encompasses but is not limited to nucleic acids or proteins which comprise a nucleic acid or amino acid sequence which differs from the nucleic acid or amino acid sequence of a reference by way of one or more substitutions, deletions and/or additions at certain positions. The variant may comprise one or more conservative substitutions. Conservative substitutions may involve, e.g., the substitution of similarly charged or uncharged amino acids.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, lipid, or the like, which is nontoxic, and which does not interact with other components of the composition in a deleterious manner.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the disclosure, the present disclosure, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology.

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD₅₀ (the dose lethal to about 50% of the population) and the ED 50 (the dose therapeutically effective in about 50% of the population). The dosage can vary depending upon the dosage form employed and the route of administration utilized. The dose ratio between toxic and therapeutic effects is the therapeutic index and can be expressed as the ratio LD₅₀/ED₅₀. In embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC₅₀ as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

As used herein, “methods of treatment” are equally applicable to use of a composition for treating the diseases or disorders described herein and/or compositions for use and/or uses in the manufacture of a medicaments for treating the diseases or disorders described herein.

### SELECTED SEQUENCES

In embodiments, the present disclosure provides for any of the sequence provided herein, including the below, and a variant sequence having at least about 90%, or at least about 93%, or at least about 95%, or at least about 97%, or at least about 98%, or at least about 99% identity thereto, or at least about 10 mutations, or at least about 9 mutations, or at least about 8 mutations, or at least about 7 mutations, or at least about 6 mutations, or at least about 5 mutations, or at least about 4 mutations, or at least about 3 mutations, or at least about 2 mutations, or at least about 1 mutation.

SEQ ID NO: 1: amino acid sequence helper transposases. Without wishing to be bound by a
particular theory, D416N (bold italic) rescues excision deficiency. The amino acids in
the DNA binding loop present (Y281-T291 and 1332-P339) were mutated, in various
embodiments.

| | |
|---|---|
| 1 | MAQHSDYSDD EFCADKLSNY SCDSDLENAS TSDEDSSDDE VMVRPRTLRR RRISSSSSDS |
| 61 | ESDIEGGREE WSHVDNPPVL EDFLGHQGLN TDAVINNIED AVKLFIGDDF FEFLVEESNR |
| 121 | YYNQNRNNFK LSKKSLKWKD ITPQEMKKFL GLIVLMGQVR KDRRDDYWTT EPWTETPYFG |
| 181 | KTMTRDRFRQ IWKAWHFNNN ADIVNESDRL CKVRPVLDYF VPKFINIYKP HQQLSLDEGI |
| 241 | VPWRGRLFFR VYNAGKIVKY GILVRLLCES DTGYICNMEI YCGEGKRLLE TIQTVVSPYT |
| 301 | DSWYHIYMDN YYNSVANCEA LMKNKFRICG TIRKNRGIPK DFQTISLKKG ETKFIRKNDI |
| 361 | LLQVWQSKKP VYLISSIHSA EMEESQNIDR TSKKKIVKPN ALIDYNKHMK GVDRADQYLS |
| 421 | YYSILRRTVK WTKRLAMYMI NCALFNSYAV YKSVRQRKMG FKMFLKQTAI HWLTDDIPED |
| 481 | MDIVPDLQPV PSTSGMRAKP PTSDPPCRLS MDMRKHTLQA IVGSGKKKNI LRRCRVCSVH |
| 541 | KLRSETRYMC KFCNIPLHKG ACFEKYHTLK NY |

SEQ ID NO: 2: amino acid sequence of a variant of a hyperactive helper with P at position
8 and R at position 13 (S8P_C13R)(572 amino acids)

| | |
|---|---|
| 1 | MAQHSDYPDD EFRADKLSNY SCDSDLENAS TSDEDSSDDE VMVRPRTLRR RRISSSSSDS |
| 61 | ESDIEGGREE WSHVDNPPVL EDFLGHQGLN TDAVINNIED AVKLFIGDDF FEELVEESNR |
| 121 | YYNQNRNNFK LSKKSLKWKD ITPQEMKKFL GLIVLMGQVR KDRRDDYWTT EPWTETPYFG |
| 181 | KTMTRDRFRQ IWKAWHFNNN ADIVNESDRL CKVRPVLDYF VPKFINIYKP HQQLSLDEGI |
| 241 | VPWRGRLFFR VYNAGKIVKY GILVRLLCES DTGYICNMEI YCGEGKRLLE TIQTVVSPYT |
| 301 | DSWYHIYMDN YYNSVANCEA LMKNKFRICG TIRKNRGIPK DFQTISLKKG ETKFIRKNDI |
| 361 | LLQVWQSKKP VYLISSIHSA EMEESQNIDR TSKKKIVKPN ALIDYNKHMK GVDRADQYLS |
| 421 | YYSILRRTVK WTKRLAMYMI NCALFNSYAV YKSVRQRKMG FKMFLKQTAI HWLTDDIPED |
| 481 | MDIVPDLQPV PSTSGMRAKP PTSDPPCRLS MDMRKHTLQA IVGSGKKKNI LRRCRVCSVH |
| 541 | KLRSETRYMC KFCNIPLHKG ACFEKYHTLK NY |

SEQ ID NO: 3: hyperactive helper Left ITR (157 bp)
The left ITR retains recognition activity when the underlined nucleotides are deleted
(80 bp).

| | |
|---|---|
| 1 | ttaacacttg gattgcggga aacgagttaa gtcggctcgc gtgaattgcg cgtactccgc |
| 61 | gggagccgtc ttaactcggt tcatatagat ttgcggtgga gtgcgggaaa cgtgtaaact |
| 121 | cgggccgatt gtaactgcgt attaccaaat atttgtt |

SEQ ID NO: 4: hyperactive helper Right ITR (212 bp)
The right ITR retains recognition activity when the underlined nucleotides are deleted
(80 bp).

| | |
|---|---|
| 1 | aattatttat gtactgaata gataaaaaaa tgtctgtgat tgaataaatt ttcatttttt |
| 61 | acacaagaaa ccgaaaattt catttcaatc gaacccatac ttcaaaagat ataggcattt |
| 121 | taaactaact ctgattttgc gcgggaaacc taaataattg cccgcgccat cttatatttt |
| 181 | ggcgggaaat tcacccgaca ccgtagtgtt aa |

SEQ ID NO: 5: nucleotide sequence of dead Cas9 DNA BINDING protein (5004 bp)

| | |
|---|---|
| 1 | ATGGACAAGA AGTACTCCAT TGGGCTCGCT ATCGGCACAA ACAGCGTCGG CTGGGCCGTC |
| 61 | ATTACGGACG AGTACAAGGT GCCGAGCAAA AAATTCAAAG TTCTGGGCAA TACCGATCGC |
| 121 | CACAGCATAA AGAAGAACCT CATTGGCGCC CTCCTGTTCG ACTCCGGGGA GACGGCCGAA |
| 181 | GCCACGCGGC TCAAAAGAAC AGCACGGCGC AGATATACCC GCAGAAAGAA TCGGATCTGC |
| 241 | TACCTGCAGG AGATCTTTAG TAATGAGATG GCTAAGGTGG ATGACTCTTT CTTCCATAGG |
| 301 | CTGGAGGAGT CCTTTTTGGT GGAGGAGGAT AAAAAGCACG AGCGCCACCC AATCTTTGGC |
| 361 | AATATCGTGG ACGAGGTGGC GTACCATGAA AAGTACCCAA CCATATATCA TCTGAGGAAG |
| 421 | AAGCTTGTAG ACAGTACTGA TAAGGCTGAC TTGCGGTTGA TCTATCTCGC GCTGGCGCAT |
| 481 | ATGATCAAAT TTCGGGGACA CTTCCTCATC GAGGGGGACC TGAACCCAGA CAACAGCGAT |
| 541 | GTCGACAAAC TCTTTATCCA ACTGGTTCAG ACTTACAATC AGCTTTTCGA AGAGAACCCG |
| 601 | ATCAACGCAT CCGGAGTTGA CGCCAAAGCA ATCCTGAGCG CTAGGCTGTC CAAATCCCGG |
| 661 | CGGCTCGAAA ACCTCATCGC ACAGCTCCCT GGGGAGAAGA AGAACGGCCT GTTTGGTAAT |
| 721 | CTTATCGCCC TGTCACTCGG GCTGACCCCC AACTTTAAAT CTAACTTCGA CCTGGCCGAA |
| 781 | GATGCCAAGC TTCAACTGAG CAAAGACACC TACGATGATG ATCTCGACAA TCTGCTGGCC |
| 841 | CAGATCGGCG ACCAGTACGC AGACCTTTTT TTGGCGGCAA AGAACCTGTC AGACGCCATT |
| 901 | CTGCTGAGTG ATATTCTGCG AGTGAACACG GAGATCACCA AAGCTCCGCT GAGCGCTAGT |
| 961 | ATGATCAAGC GCTATGATGA GCACCACCAA GACTTGACTT TGCTGAAGGC CCTTGTCAGA |
| 1021 | CAGCAACTGC CTGAGAAGTA CAAGGAAATT TTCTTCGATC AGTCTAAAAA TGGCTACGCC |
| 1081 | GGATACATTG ACGGCGGAGC AAGCCAGGAG GAATTTTACA AATTTATTAA GCCCATCTTG |
| 1141 | GAAAAAATGG ACGGCACCGA GGAGCTGCTG GTAAAGCTTA ACAGAGAAGA TCTGTTGCGC |
| 1201 | AAACAGCGCA CTTTCGACAA TGGAAGCATC CCCCACCAGA TTCACCTGGG CGAACTGCAC |
| 1261 | GCTATCCTCA GGCGGCAAGA GGATTTCTAC CCCTTTTTGA AAGATAACAG GGAAAAGATT |
| 1321 | GAGAAAATCC TCACATTTCG GATACCCTAC TATGTAGGCC CCCTCGCCCG GGGAAATTCC |
| 1381 | AGATTCGCGT GGATGACTCG CAAATCAGAA GAGACCATCA CTCCCTGGAA CTTCGAGGAA |
| 1441 | GTCGTGGATA AGGGGGCCTC TGCCCAGTCC TTCATCGAAA GGATGACTAA CTTTGATAAA |
| 1501 | AATCTGCCTA ACGAAAAGGT GCTTCCTAAA CACTCTCTGC TGTACGAGTA CTTCACAGTT |
| 1561 | TATAACGAGC TCACCAAGGT CAAATACGTC ACAGAAGGGA TGAGAAAGCC AGCATTCCTG |
| 1621 | TCTGGAGAGC AGAAGAAAGC TATCGTGGAC CTCCTCTTCA AGACGAACCG GAAAGTTACC |
| 1681 | GTGAAACAGC TCAAAGAAGA CTATTTCAAA AAGATTGAAT GTTTCGACTC TGTTGAAATC |
| 1741 | AGCGGAGTGG AGGATCGCTT CAACGCATCC CTGGGAACGT ATCACGATCT CCTGAAAATC |
| 1801 | ATTAAAGACA AGGACTTCCT GGACAATGAG GAGAACGAGG ACATTCTTGA GGACATTGTC |
| 1861 | CTCACCCTTA CGTTGTTTGA AGATAGGGAG ATGATTGAAG AACGCTTGAA AACTTACGCT |
| 1921 | CATCTCTTCG ACGACAAAGT CATGAAACAG CTCAAGAGGC GCCGATATAC AGGATGGGGG |
| 1981 | CGGCTGTCAA GAAAACTGAT CAATGGGATC CGAGACAAGC AGAGTGGAAA GACAATCCTG |
| 2041 | GATTTTCTTA AGTCCGATGG ATTTGCCAAC CGGAACTTCA TGCAGTTGAT CCATGATGAC |
| 2101 | TCTCTCACCT TTAAGGAGGA CATCCAGAAA GCACAAGTTT CTGGCCAGGG GGACAGTCTT |
| 2161 | CACGAGCACA TCGCTAATCT TGCAGGTAGC CCAGCTATCA AAAAGGGAAT ACTGCAGACC |
| 2221 | GTTAAGGTCG TGGATGAACT CGTCAAAGTA ATGGGAAGGC ATAAGCCCGA GAATATCGTT |
| 2281 | ATCGAGATGG CCCGAGAGAA CCAAACTACC CAGAAGGGAC AGAAGAACAG TAGGGAAAGG |
| 2341 | ATGAAGAGGA TTGAAGAGGG TATAAAAGAA CTGGGGTCCC AAATCCTTAA GGAACACCCA |
| 2401 | GTTGAAAACA CCCAGCTTCA GAATGAGAAG CTCTACCTGT ACTACCTGCA GAACGGCAGG |
| 2461 | GACATGTACG TGGATCAGGA ACTGGACATC AATCGGCTCT CCGACTACGA CGTGGCTGCT |
| 2521 | ATCGTGCCCC AGTCTTTTCT CAAAGATGAT TCTATTGATA ATAAAGTGTT GACAAGATCC |
| 2581 | GATAAAGCTA GAGGGAAGAG TGATAACGTC CCCTCAGAAG AAGTTGTCAA GAAAATGAAA |
| 2641 | AATTATTGGC GGCAGCTGCT GAACGCCAAA CTGATCACAC AACGGAAGTT CGATAATCTG |
| 2701 | ACTAAGGCTG AACGAGGTGG CCTGTCTGAG TTGGATAAAG CCGGCTTCAT CAAAAGGCAG |
| 2761 | CTTGTTGAGA CACGCCAGAT CACCAAGCAC GTGGCCCAAA TTCTCGATTC ACGCATGAAC |
| 2821 | ACCAAGTACG ATGAAAATGA CAAACTGATT CGAGAGGTGA AAGTTATTAC TCTGAAGTCT |
| 2881 | AAGCTGGTCT CAGATTTCAG AAAGGACTTT CAGTTTTATA AGGTGAGAGA GATCAACAAT |
| 2941 | TACCACCATG CGCATGATGC CTACCTGAAT GCAGTGGTAG GCACTGCACT TATCAAAAAA |
| 3001 | TATCCCAAGC TTGAATCTGA ATTTGTTTAC GGAGACTATA AAGTGTACGA TGTTAGGAAA |
| 3061 | ATGATCGCAA AGTCTGAGCA GGAAATAGGC AAGGCCACCG CTAAGTACTT CTTTTACAGC |
| 3121 | AATATTATGA ATTTTTTCAA GACCGAGATT ACACTGGCCA ATGGAGAGAT TCGGAAGCGA |
| 3181 | CCACTTATCG AAACAAACGG AGAAACAGGA GAAATCGTGT GGGACAAGGG TAGGGATTTC |
| 3241 | GCGACAGTCC GGAAGGTCCT GTCCATGCCG CAGGTGAACA TCGTTAAAAA GACCGAAGTA |
| 3301 | CAGACCGGAG GCTTCTCCAA GGAAAGTATC CTCCCGAAAA GGAACAGCGA CAAGCTGATC |
| 3361 | GCACGCAAAA AAGATTGGGA CCCCAAGAAA TACGGCGGAT TCGATTCTCC TACAGTCGCT |
| 3421 | TACAGTGTAC TGGTTGTGGC CAAAGTGGAG AAAGGGAAGT CTAAAAAACT CAAAAGCGTC |
| 3481 | AAGGAACTGC TGGGCATCAC AATCATGGAG CGATCAAGCT TCGAAAAAAA CCCCATCGAC |
| 3541 | TTTCTGGAGG CGAAAGGATA TAAAGAGGTC AAAAAAGACC TCATCATTAA GCTTCCCAAG |
| 3601 | TACTCTCTCT TTGAGCTTGA AAACGGCCGG AAACGAATGC TCGCTAGTGC GGGCGAGCTG |
| 3661 | CAGAAAGGTA ACGAGCTGGC ACTGCCCTCT AAATACGTTA ATTTCTTGTA TCTGGCCAGC |
| 3721 | CACTATGAAA AGCTCAAAGG GTCTCCCGAA GATAATGAGC AGAAGCAGCT GTTCGTGGAA |
| 3781 | CAACACAAAC ACTACCTTGA TGAGATCATC GAGCAAATAA GCGAATTCTC CAAAAGAGTG |
| 3841 | ATCCTCGCCG ACGCTAACCT CGATAAGGTG CTTTCTGCTT ACAATAAGCA CAGGGATAAG |
| 3901 | CCCATCAGGG AGCAGGCAGA AAACATTATC CACTTGTTTA CTCTGACCAA CTTGGGCGCG |
| 3961 | CCTGCAGCCT TCAAGTACTT CGACACCACC ATAGACAGAA AGCGGTACAC CTCTACAAAG |
| 4021 | GAGGTCCTGG ACGCCACACT GATTCATCAG TCAATTACGG GGCTCTATGA AACAAGAATC |
| 4081 | GACCTCTCTC AGCTCGGTGG AGAC |

SEQ ID NO: 6: amino acid sequence of dead Cas9 DNA BINDING protein (1368 amino acids)

| | |
|---|---|
| 1 | MDKKYSIGLA IGTNSVGWAV ITDEYKVPSK KFKVLGNTDR HSIKKNLIGA LLFDSGETAE |
| 61 | ATRLKRTARR RYTRRKNRIC YLQEIFSNEM AKVDDSFFHR LEESFLVEED KKHERHPIFG |
| 121 | NIVDEVAYHE KYPTIYHLRK KLVDSTDKAD LRLIYLALAH MIKFRGHFLI EGDLNPDNSD |
| 181 | VDKLFIQLVQ TYNQLFEENP INASGVDAKA ILSARLSKSR RLENLIAQLP GEKKNGLFGN |
| 241 | LIALSLGLTP NFKSNFDLAE DAKLQLSKDT YDDDLDNLLA QIGDQYADLF LAAKNLSDAI |
| 301 | LLSDILRVNT EITKAPLSAS MIKRYDEHHQ DLTLLKALVR QQLPEKYKEI FFDQSKNGYA |
| 361 | GYIDGGASQE EFYKFIKPIL EKMDGTEELL VKLNREDLLR KQRTFDNGSI PHQIHLGELH |
| 421 | AILRRQEDFY PFLKDNRFKI EKILTFRIPY YVGPLARGNS RFAWMTRKSE ETITPWNFEE |
| 481 | VVDKGASAQS FIERMTNFDK NLPNEKVLPK HSLLYEYFTV YNELTKVKYV TEGMRKPAFL |
| 541 | SGEQKKAIVD LLFKTNRKVT VKQLKEDYFK KIECFDSVEI SGVEDRFNAS LGTYHDLLKI |
| 601 | IKDKDFLDNE ENEDILEDIV LTLTLFEDRE MIEERLKTYA HLFDDKVMKQ LKRRRYTGWG |
| 661 | RLSRKLINGI RDKQSGKTIL DFLKSDGFAN RNFMQLIHDD SLTFKEDIQK AQVSGQGDSL |
| 721 | HEHIANLAGS PAIKKGILQT VKVVDELVKV MGRHKPENIV IEMARENQTT QKGQKNSRER |
| 781 | MKRIEEGIKE LGSQILKEHP VENTQLQNEK LYLYYLQNGR DMYVDQELDI NRLSDYDVAA |
| 841 | IVPQSFLKDD SIDNKVLTRS DKARGKSDNV PSEEVVKKMK NYWRQLLNAK LITQRKFDNL |
| 901 | TKAERGGLSE LDKAGFIKRQ LVETRQITKH VAQILDSRMN TKYDENDKLI REVKVITLKS |
| 961 | KLVSDFRKDF QFYKVREINN YHHAHDAYLN AVVGTALIKK YPKLESEFVY GDYKVYDVRK |
| 1021 | MIAKSEQEIG KATAKYFFYS NIMNFFKTEI TLANGEIRKR PLIETNGETG EIVWDKGRDF |
| 1081 | ATVRKVLSMP QVNIVKKTEV QTGGFSKESI LPKRNSDKLI ARKKDWDPKK YGGFDSPTVA |
| 1141 | YSVLVVAKVE KGKSKKLKSV KELLGITIME RSSFEKNPID FLEAKGYKEV KKDLIIKLPK |
| 1201 | YSLFELENGR KRMLASAGEL QKGNELALPS KYVNFLYLAS HYEKLKGSPE DNEQKQLFVE |
| 1261 | QHKHYLDEII EQISEFSKRV ILADANLDKV LSAYNKHRDK PIREQAENII HLFTLTNLGA |
| 1321 | PAAFKYFDTT IDRKRYTSTK EVLDATLIHQ SITGLYETRI DLSQLGGD |

SEQ ID NO: 12: amino acid sequence of E. coli TnsD (508 amino acids)

| | |
|---|---|
| 1 | MRNFPVPYSN ELIYSTIARA GVYQGIVSPK QLLDEVYGNR KVVATLGLPS HLGVIARHLH |
| 61 | QTGRYAVQQL IYEHTLFPLY APFVGKERRD EAIRLMEYQA QGAVHLMLGV AASRVKSDNR |
| 121 | FRYCPDCVAL QLNRYGEAFW QRDWYLPALP YCPKHGALVF FDRAVDDHRH QFWALGHTEL |
| 181 | LSDYPKDSLS QLTALAAYIA PLLDAPRAQE LSPSLEQWTL FYQRLAQDLG LTKSKHIRHD |
| 241 | LVAERVRQTF SDEALEKLDL KLAENKDTCW LKSIFRKHRK AFSYLQHSIV WQALLPKLTV |
| 301 | IEALQQASAL TEHSITTRPV SQSVQPNSED LSVKHKDWQQ LVHKYQGIKA ARQSLEGGVL |
| 361 | YAWLYRHDRD WLVHWNQQHQ QERLAPAPRV DWNQRDRIAV RQLLRIIKRL DSSLDHPRAT |
| 421 | SSWLLKQTPN GTSLAKNLQK LPLVALCLKR YSESVEDYQI RRISQAFIKL KQEDVELRRW |
| 481 | RLLRSATLSK ERITEEAQRF LEMVYGEE |

SEQ ID NO: 504: Myositis lucifugus (hyperactive helper) amino acid sequence (N1, amino
acid 2-35 deletion). 538 aa

| | |
|---|---|
| 1 | MSSDDEVMVR PRTLRRRRIS SSSSDSESDI EGGREEWSHV DNPPVLEDFL GHQGLNTDAV |
| 61 | INNIEDAVKL FIGDDFFEFL VEESNRYYNQ NRNNFKLSKK SLKWKDITPQ EMKKELGLIV |
| 121 | LMGQVRKDRR DDYWTTEPWT ETPYFGKTMT RDRFRQIWKA WHENNNADIV NESDRLCKVR |
| 181 | PVLDYFVPKF INIYKPHQQL SLDEGIVPWR GRLFFRVYNA GKIVKYGILV RLLCESDTGY |
| 241 | ICNMEIYCGE GKRLLETIQT VVSPYTDSWY HIYMDNYYNS VANCEALMKN KFRICGTIRK |
| 301 | NRGIPKDFQT ISLKKGETKF IRKNDILLQV WQSKKPVYLI SSIHSAEMEE SQNIDRTSKK |
| 361 | KIVKPNALID YNKHMKGVDR ADQYLSYYSI LRRTVKWTKR LAMYMINCAL FNSYAVYKSV |
| 421 | RQRKMGFKMF LKQTAIHWLT DDIPEDMDIV PDLQPVPSTS GMRAKPPTSD PPCRLSMDMR |
| 481 | KHTLQAIVGS GKKKNILRRC RVCSVHKLRS ETRYMCKFCN IPLHKGACFE KYHTLKNY |

SEQ ID NO: 506: Myositis lucifugus (hyperactive helper) amino acid sequence (N2, amino
acid 2-45 deletion). 528 aa

| | |
|---|---|
| 1 | MRTLRRRRIS SSSSDSESDI EGGREEWSHV DNPPVLEDFL GHQGLNTDAV INNIEDAVKL |
| 61 | FIGDDFFEEL VEESNRYYNQ NRNNFKLSKK SLKWKDITPQ EMKKELGLIV LMGQVRKDRR |
| 121 | DDYWTTEPWT ETPYFGKTMT RDRFRQIWKA WHENNNADIV NESDRLCKVR PVLDYFVPKF |
| 181 | INIYKPHQQL SLDEGIVPWR GRLFFRVYNA GKIVKYGILV RLLCESDTGY ICNMEIYCGE |
| 241 | GKRLLETIQT VVSPYTDSWY HIYMDNYYNS VANCEALMKN KFRICGTIRK NRGIPKDFQT |
| 301 | ISLKKGETKF IRKNDILLQV WQSKKPVYLI SSIHSAEMEE SQNIDRTSKK KIVKPNALID |
| 361 | YNKHMKGVDR ADQYLSYYSI LRRTVKWTKR LAMYMINCAL FNSYAVYKSV RQRKMGFKME |
| 421 | LKQTAIHWLT DDIPEDMDIV PDLQPVPSTS GMRAKPPTSD PPCRLSMDMR KHTLQAIVGS |
| 481 | GKKKNILRRC RVCSVHKLRS ETRYMCKFCN IPLHKGACFE KYHTLKNY |

SEQ ID NO: 508: Myositis lucifugus (hyperactive helper) amino acid sequence (N3, amino
acid 2-68 deletion) 505 aa

| | |
|---|---|
| 1 | MEEWSHVDNP PVLEDFLGHQ GINTDAVINN IEDAVKLFIG DDFFEFLVEE SNRYYNQNRN |
| 61 | NFKLSKKSLK WKDITPQEMK KFLGLIVLMG QVRKDRRDDY WTTEPWTETP YFGKTMTRDR |
| 121 | FRQIWKAWHF NNNADIVNES DRLCKVRPVL DYFVPKFINI YKPHQQLSLD EGIVPWRGRL |
| 181 | FFRVYNAGKI VKYGILVRLL CESDTGYICN MEIYCGEGKR LLETIQTVVS PYTDSWYHIY |
| 241 | MDNYYNSVAN CEALMKNKER ICGTIRKNRG IPKDFQTISL KKGETKFIRK NDILLQVWQS |
| 301 | KKPVYLISSI HSAEMEESQN IDRTSKKKIV KPNALIDYNK HMKGVDRADQ YLSYYSILRR |
| 361 | TVKWTKRLAM YMINCALENS YAVYKSVRQR KMGFKMELKQ TAIHWLTDDI PEDMDIVPDL |
| 421 | QPVPSTSGMR AKPPTSDPPC RLSMDMRKHT LQAIVGSGKK KNILRRCRVC SVHKLRSETR |
| 481 | YMCKFCNIPL HKGACFEKYH TLKNY |

SEQ ID NO: 882: E2C ZnF inserted after the G66 residue relative to SEQ ID NO: 1 of MLT
with mutations, S8P_C13R_R333K_K334A_N335A_R336K_D416N substitutions relative to SEQ
ID NO: 1
ATGCCGAAAAAAAAACGAAAGGTGTACCCCTACGATGTACCGGACTATGCAGGAAGCGCCCAGCACAGCGACTACCC
CGACGACGAGTTCAGAGCCGATAAGCTGAGTAACTACAGCTGCGACAGCGACCTGGAAAACGCCAGCACATCCGACG
AGGACAGCTCTGACGACGAGGTGATGGTGCGGCCCAGAACCCTGAGACGGAGAAGAATCAGCAGCTCTAGCAGCGAC
TCTGAATCCGACATCGAGGGCGGAGGTCTCGAACCAGGCGAGAAGCCTTATGCCTGTCCTGAGTGTGGCAAATCCTT
CTCAAGAAAAGACTCTCTGGTTAGACACCAGAGAACACATACAGGGGAGAAACCCTATAAATGCCCCGAATGCGGAA
AGTCCTTTTCCCAGAGCGGCGATCTCCGGAGGCATCAGAGAACTCATACAGGCGAGAAACCATATAAGTGCCCCGAG
TGTGGGAAATCCTTTTCCGATTGTAGAGACCTGGCCAGACATCAAAGGACACATACAGGCAAGAAGACCGGAGGTGG
CCGGGAAGAGTGGAGCCACGTGGACAACCCTCCTGTTCTGGAAGATTTTCTGGGCCATCAGGGCCTGAACACCGACG
CCGTGATCAACAACATCGAGGATGCCGTGAAGCTGTTCATAGGAGATGATTTCTTTGAGTTCCTGGTCGAGGAATCC
AACCGCTATTACAACCAGAATAGAAACAACTTCAAGCTGAGCAAGAAAAGCCTGAAGTGGAAGGACATCACCCCTCA
GGAGATGAAAAAGTTCCTGGGACTGATCGTTCTGATGGGACAGGTGCGGAAGGACAGAAGGGATGATTACTGGACAA
CCGAACCTTGGACCGAGACCCCTTACTTTGGCAAGACCATGACCAGAGACAGATTCAGACAGATCTGGAAAGCCTGG
CACTTCAACAACAATGCTGATATCGTGAACGAGTCTGATAGACTGTGTAAAGTGCGGCCAGTGTTGGATTACTTCGT
GCCTAAGTTCATCAACATCTATAAGCCTCACCAGCAGCTGAGCCTGGATGAAGGCATCGTGCCCTGGCGGGGCAGAC
TGTTCTTCAGAGTGTACAATGCTGGCAAGATCGTCAAATACGGCATCCTGGTGCGCCTTCTGTGCGAGAGCGATACA
GGCTACATCTGTAATATGGAAATCTACTGCGGCGAGGGCAAAAGACTGCTGGAAACCATCCAGACCGTCGTTTCCCC
TTATACCGACAGCTGGTACCACATCTACATGGACAACTACTACAATTCTGTGGCCAACTGCGAGGCCCTGATGAAGA
ACAAGTTTAGAATCTGCGGCACAATCAAGGCCGCCAAGGGCATCCCTAAGGACTTCCAGACCATCTCTCTGAAGAAG
GGCGAAACCAAGTTCATCAGAAAGAACGACATCCTGCTCCAAGTGTGGCAGTCCAAGAAACCCGTGTACCTGATCAG
CAGCATCCATAGCGCCGAGATGGAAGAAAGCCAGAACATCGACAGAACAAGCAAGAAGAAGATCGTGAAGCCCAATG
CTCTGATCGACTACAACAAGCACATGAAAGGCGTGGACCGGGCCAACCAGTACCTGTCTTATTACTCTATCCTGAGA
AGAACAGTGAAATGGACCAAGAGACTGGCCATGTACATGATCAATTGCGCCCTGTTCAACAGCTACGCCGTGTACAA
GTCCGTGCGACAAAGAAAAATGGGATTCAAGATGTTCCTGAAGCAGACAGCCATCCACTGGCTGACAGACGACATTC
CTGAGGACATGGACATTGTGCCAGATCTGCAACCTGTGCCCAGCACCTCTGGTATGAGAGCTAAGCCTCCCACCAGC
GATCCTCCATGTAGACTGAGCATGGACATGCGGAAGCACACCCTGCAGGCCATCGTCGGCAGCGGCAAGAAGAAGAA
CATCCTTAGACGGTGCAGGGTGTGCAGCGTGCACAAGCTGCGGAGCGAGACTCGGTACATGTGCAAGTTTTGCAACA
TTCCCCTGCACAAGGGAGCCTGCTTCGAGAAGTACCACACCCTGAAGAATTAC
SEQ ID NO: 883: E2C ZnF inserted after the G66 residue relative to SEQ ID NO: 1 of MLT
with mutations, S8P_C13R_R333K_N335A_R336K_D416N substitutions relative to SEQ ID NO: 1
ATGCCGAAAAAAAAACGAAAGGTGTACCCCTACGATGTACCGGACTATGCAGGAAGCGCCCAGCACAGCGACTACCC
CGACGACGAGTTCAGAGCCGATAAGCTGAGTAACTACAGCTGCGACAGCGACCTGGAAAACGCCAGCACATCCGACG
AGGACAGCTCTGACGACGAGGTGATGGTGCGGCCCAGAACCCTGAGACGGAGAAGAATCAGCAGCTCTAGCAGCGAC
TCTGAATCCGACATCGAGGGCGGAGGTCTCGAACCAGGCGAGAAGCCTTATGCCTGTCCTGAGTGTGGCAAATCCTT
CTCAAGAAAAGACTCTCTGGTTAGACACCAGAGAACACATACAGGGGAGAAACCCTATAAATGCCCCGAATGCGGAA
AGTCCTTTTCCCAGAGCGGCGATCTCCGGAGGCATCAGAGAACTCATACAGGCGAGAAACCATATAAGTGCCCCGAG
TGTGGGAAATCCTTTTCCGATTGTAGAGACCTGGCCAGACATCAAAGGACACATACAGGCAAGAAGACCGGAGGTGG
CCGGGAAGAGTGGAGCCACGTGGACAACCCTCCTGTTCTGGAAGATTTTCTGGGCCATCAGGGCCTGAACACCGACG
CCGTGATCAACAACATCGAGGATGCCGTGAAGCTGTTCATAGGAGATGATTTCTTTGAGTTCCTGGTCGAGGAATCC
AACCGCTATTACAACCAGAATAGAAACAACTTCAAGCTGAGCAAGAAAAGCCTGAAGTGGAAGGACATCACCCCTCA
GGAGATGAAAAAGTTCCTGGGACTGATCGTTCTGATGGGACAGGTGCGGAAGGACAGAAGGGATGATTACTGGACAA
CCGAACCTTGGACCGAGACCCCTTACTTTGGCAAGACCATGACCAGAGACAGATTCAGACAGATCTGGAAAGCCTGG
CACTTCAACAACAATGCTGATATCGTGAACGAGTCTGATAGACTGTGTAAAGTGCGGCCAGTGTTGGATTACTTCGT
GCCTAAGTTCATCAACATCTATAAGCCTCACCAGCAGCTGAGCCTGGATGAAGGCATCGTGCCCTGGCGGGGCAGAC
TGTTCTTCAGAGTGTACAATGCTGGCAAGATCGTCAAATACGGCATCCTGGTGCGCCTTCTGTGCGAGAGCGATACA
GGCTACATCTGTAATATGGAAATCTACTGCGGCGAGGGCAAAAGACTGCTGGAAACCATCCAGACCGTCGTTTCCCC
TTATACCGACAGCTGGTACCACATCTACATGGACAACTACTACAATTCTGTGGCCAACTGCGAGGCCCTGATGAAGA
ACAAGTTTAGAATCTGCGGCACAATCAAGAAAGCCAAGGGCATCCCTAAGGACTTCCAGACCATCTCTCTGAAGAAG
GGCGAAACCAAGTTCATCAGAAAGAACGACATCCTGCTCCAAGTGTGGCAGTCCAAGAAACCCGTGTACCTGATCAG
CAGCATCCATAGCGCCGAGATGGAAGAAAGCCAGAACATCGACAGAACAAGCAAGAAGAAGATCGTGAAGCCCAATG
CTCTGATCGACTACAACAAGCACATGAAAGGCGTGGACCGGGCCAACCAGTACCTGTCTTATTACTCTATCCTGAGA
AGAACAGTGAAATGGACCAAGAGACTGGCCATGTACATGATCAATTGCGCCCTGTTCAACAGCTACGCCGTGTACAA
GTCCGTGCGACAAAGAAAAATGGGATTCAAGATGTTCCTGAAGCAGACAGCCATCCACTGGCTGACAGACGACATTC
CTGAGGACATGGACATTGTGCCAGATCTGCAACCTGTGCCCAGCACCTCTGGTATGAGAGCTAAGCCTCCCACCAGC
GATCCTCCATGTAGACTGAGCATGGACATGCGGAAGCACACCCTGCAGGCCATCGTCGGCAGCGGCAAGAAGAAGAA
CATCCTTAGACGGTGCAGGGTGTGCAGCGTGCACAAGCTGCGGAGCGAGACTCGGTACATGTGCAAGTTTTGCAACA
TTCCCCTGCACAAGGGAGCCTGCTTCGAGAAGTACCACACCCTGAAGAATTAC
SEQ ID NO: 884: HoD4 TALE inserted after the G66 residue relative to SEQ ID NO: 1 of MLT
with mutation: R333K_N335A_R336K_D416N substitutions relative to SEQ ID NO: 1
ATGGCGCCTAAGAAGAAGCGCAAGGTGGGCGGAGCCCAGCACAGCGACTACCCCGACGACGAGTTCAGAGCCGATAA
GCTGAGTAACTACAGCTGCGACAGCGACCTGGAAAACGCCAGCACATCCGACGAGGACAGCTCTGACGACGAGGTGA
TGGTGCGGCCCAGAACCCTGAGACGGAGAAGAATCAGCAGCTCTAGCAGCGACTCTGAATCCGACATCGAGGGCGGT
GGTGGGTCTGTTGACCTCCGGACTTTGGGTTACTCCCAACAGCAACAAGAGAAGATTAAACCTAAGGTGCGCTCAAC
AGTGGCGCAGCATCATGAGGCACTGGTTGGTCACGGGTTCACACATGCCCATATAGTAGCACTTTCACAACATCCCG
CTGCACTCGGAACGGTTGCAGTAAAGTACCAAGACATGATTGCCGCATTGCCGGAAGCAACTCATGAGGCCATTGTC
GGAGTAGGCAAATACCACGGTGCTAGAGCTCTCGAAGCACTCCTCACTGTGGCTGGCGAATTGAGGGGGCCCCCCCT
TCAACTTGATACTGGACAGCTGTTGAAGATAGCGAAACGAGGGGGAGTAACCGCAGTCGAGGCCGTCCACGCGTGGC
GAAATGCATTGACGGGAGCGCCTCTCAACCTTACTCCAGATCAGGTTGTGGCCATAGCGAGCAACATCGGAGGTAAG
CAAGCACTGGAAACTGTTCAACGATTGCTCCCAGTACTCTGCCAAGACCACGGATTGACCCCAGAACAGGTTGTGGC
TATTGCCAGCAACGGCGGCGGTAAACAGGCTCTGGAAACTGTGCAGCGACTCTTGCCGGTGTTGTGCCAAGCTCATG
GCCTTACGCCAGACCAGGTTGTTGCTATCGCTAGCAACATCGGAGGCAAACAGGCACTTGAAACGGTCCAACGCCTC
TTGCCAGTATTGTGCCAGGCACATGGCCTCACCCCCGCCCAGGTTGTGGCCATCGCAAACAATAACGGGGGCAAGCA
GGCTTTGGAGACAGTCCAACGATTGTTGCCGGTACTTTGTCAGGACCACGGGCTGACCCCAGATCAAGTCGTAGCAA
TCGCTAGCAACATCGGCGGGAAGCAGGCCCTGGAAACGGTGCAGAGGCTCCTCCCAGTGCTCTGCCAAGATCATGGC
CTCACCCCAGAGCAAGTGGTCGCAATCGCAAACAATAACGGTGGCAAGCAAGCCCTCGAAACTGTGCAAAGGTTGCT
GCCAGTTCTCTGTCAAGCCCACGGTTTGACACCGGATCAGGTAGTGGCTATCGCTAGCAACGGCGGTGGCAAGCAAG
CTCTCGAGACAGTCCAGAGGCTTTTGCCAGTACTGTGCCAAGCGCACGGTCTCACTCCTGCGCAAGTTGTTGCTATA
GCAAACAATAACGGTGGAAAGCAAGCTCTTGAGACTGTGCAGCGCCTGTTGCCCGTCCTCTGTCAGGACCACGGTTT
GACTCCAGACCAAGTTGTCGCCATTGCAAGCAACGGCGGTGGACGCCCTGCACTGGAGTCCATCGTCGCACAACTGT
CTAGGCCGGACCCCGCCCTGGCTGCCCTTACTAATGATCACTTGGTTGCATTGGCGTGTCTGGGCGGTCGGCCAGCG
CTGGACGCAGTCAAGAAGGGACTCCCCCACGCCCCTGCACTTATAAAACGCACTAACAGGAGAATACCAGAACGGAC
GAGTCATCGAGTCGCTGGAGGGGGCGGTAGCGGCCGGGAAGAGTGGAGCCACGTGGACAACCCTCCTGTTCTGGAAG
ATTTTCTGGGCCATCAGGGCCTGAACACCGACGCCGTGATCAACAACATCGAGGATGCCGTGAAGCTGTTCATAGGA
GATGATTTCTTTGAGTTCCTGGTCGAGGAATCCAACCGCTATTACAACCAGAATAGAAACAACTTCAAGCTGAGCAA
GAAAAGCCTGAAGTGGAAGGACATCACCCCTCAGGAGATGAAAAAGTTCCTGGGACTGATCGTTCTGATGGGACAGG
TGCGGAAGGACAGAAGGGATGATTACTGGACAACCGAACCTTGGACCGAGACCCCTTACTTTGGCAAGACCATGACC
AGAGACAGATTCAGACAGATCTGGAAAGCCTGGCACTTCAACAACAATGCTGATATCGTGAACGAGTCTGATAGACT
GTGTAAAGTGCGGCCAGTGTTGGATTACTTCGTGCCTAAGTTCATCAACATCTATAAGCCTCACCAGCAGCTGAGCC
TGGATGAAGGCATCGTGCCCTGGCGGGGCAGACTGTTCTTCAGAGTGTACAATGCTGGCAAGATCGTCAAATACGGC
ATCCTGGTGCGCCTTCTGTGCGAGAGCGATACAGGCTACATCTGTAATATGGAAATCTACTGCGGCGAGGGCAAAAG
ACTGCTGGAAACCATCCAGACCGTCGTTTCCCCTTATACCGACAGCTGGTACCACATCTACATGGACAACTACTACA
ATTCTGTGGCCAACTGCGAGGCCCTGATGAAGAACAAGTTTAGAATCTGCGGCACAATCAGAAAAAACAGAGGCATC
CCTAAGGACTTCCAGACCATCTCTCTGAAGAAGGGCGAAACCAAGTTCATCAGAAAGAACGACATCCTGCTCCAAGT
GTGGCAGTCCAAGAAACCCGTGTACCTGATCAGCAGCATCCATAGCGCCGAGATGGAAGAAAGCCAGAACATCGACA
GAACAAGCAAGAAGAAGATCGTGAAGCCCAATGCTCTGATCGACTACAACAAGCACATGAAAGGCGTGGACCGGGCC
GACCAGTACCTGTCTTATTACTCTATCCTGAGAAGAACAGTGAAATGGACCAAGAGACTGGCCATGTACATGATCAA
TTGCGCCCTGTTCAACAGCTACGCCGTGTACAAGTCCGTGCGACAAAGAAAAATGGGATTCAAGATGTTCCTGAAGC
AGACAGCCATCCACTGGCTGACAGACGACATTCCTGAGGACATGGACATTGTGCCAGATCTGCAACCTGTGCCCAGC
ACCTCTGGTATGAGAGCTAAGCCTCCCACCAGCGATCCTCCATGTAGACTGAGCATGGACATGCGGAAGCACACCCT
GCAGGCCATCGTCGGCAGCGGCAAGAAGAAGAACATCCTTAGACGGTGCAGGGTGTGCAGCGTGCACAAGCTGCGGA
GCGAGACTCGGTACATGTGCAAGTTTTGCAACATTCCCCTGCACAAGGGAGCCTGCTTCGAGAAGTACCACACCCTG
AAGAATTAC
SEQ ID NO: 885: ALFA tag inserted after the G66 residue relative to SEQ ID NO: 1 of MLT
ATGCCGAAAAAAAAACGAAAGGTGTACCCCTACGATGTACCGGACTATGCAGGAAGCGCCCAGCACAGCGACTACCC
CGACGACGAGTTCAGAGCCGATAAGCTGAGTAACTACAGCTGCGACAGCGACCTGGAAAACGCCAGCACATCCGACG
AGGACAGCTCTGACGACGAGGTGATGGTGCGGCCCAGAACCCTGAGACGGAGAAGAATCAGCAGCTCTAGCAGCGAC
TCTGAATCCGACATCGAGGGCTCCAGACTGGAAGAGGAACTGAGAAGAAGGCTCACAGAAGGCCGGGAAGAGTGGAG
CCACGTGGACAACCCTCCTGTTCTGGAAGATTTTCTGGGCCATCAGGGCCTGAACACCGACGCCGTGATCAACAACA
TCGAGGATGCCGTGAAGCTGTTCATAGGAGATGATTTCTTTGAGTTCCTGGTCGAGGAATCCAACCGCTATTACAAC
CAGAATAGAAACAACTTCAAGCTGAGCAAGAAAAGCCTGAAGTGGAAGGACATCACCCCTCAGGAGATGAAAAAGTT
CCTGGGACTGATCGTTCTGATGGGACAGGTGCGGAAGGACAGAAGGGATGATTACTGGACAACCGAACCTTGGACCG
AGACCCCTTACTTTGGCAAGACCATGACCAGAGACAGATTCAGACAGATCTGGAAAGCCTGGCACTTCAACAACAAT
GCTGATATCGTGAACGAGTCTGATAGACTGTGTAAAGTGCGGCCAGTGTTGGATTACTTCGTGCCTAAGTTCATCAA
CATCTATAAGCCTCACCAGCAGCTGAGCCTGGATGAAGGCATCGTGCCCTGGCGGGGCAGACTGTTCTTCAGAGTGT
ACAATGCTGGCAAGATCGTCAAATACGGCATCCTGGTGCGCCTTCTGTGCGAGAGCGATACAGGCTACATCTGTAAT
ATGGAAATCTACTGCGGCGAGGGCAAAAGACTGCTGGAAACCATCCAGACCGTCGTTTCCCCTTATACCGACAGCTG
GTACCACATCTACATGGACAACTACTACAATTCTGTGGCCAACTGCGAGGCCCTGATGAAGAACAAGTTTAGAATCT
GCGGCACAATCAGAAAAAACAGAGGCATCCCTAAGGACTTCCAGACCATCTCTCTGAAGAAGGGCGAAACCAAGTTC
ATCAGAAAGAACGACATCCTGCTCCAAGTGTGGCAGTCCAAGAAACCCGTGTACCTGATCAGCAGCATCCATAGCGC
CGAGATGGAAGAAAGCCAGAACATCGACAGAACAAGCAAGAAGAAGATCGTGAAGCCCAATGCTCTGATCGACTACA
ACAAGCACATGAAAGGCGTGGACCGGGCCGACCAGTACCTGTCTTATTACTCTATCCTGAGAAGAACAGTGAAATGG
ACCAAGAGACTGGCCATGTACATGATCAATTGCGCCCTGTTCAACAGCTACGCCGTGTACAAGTCCGTGCGACAAAG
AAAAATGGGATTCAAGATGTTCCTGAAGCAGACAGCCATCCACTGGCTGACAGACGACATTCCTGAGGACATGGACA
TTGTGCCAGATCTGCAACCTGTGCCCAGCACCTCTGGTATGAGAGCTAAGCCTCCCACCAGCGATCCTCCATGTAGA
CTGAGCATGGACATGCGGAAGCACACCCTGCAGGCCATCGTCGGCAGCGGCAAGAAGAAGAACATCCTTAGACGGTG
CAGGGTGTGCAGCGTGCACAAGCTGCGGAGCGAGACTCGGTACATGTGCAAGTTTTGCAACATTCCCCTGCACAAGG
GAGCCTGCTTCGAGAAGTACCACACCCTGAAGAATTAC
SEQ ID NO: 886: HoD4 TALE fused to the recombinant variable domains of heavy-chain-only
antibodies (VHH) (NANOBODY) bound to an ALFA-tag peptide (NbALFA)
ATGGCGCCTAAGAAGAAGCGCAAGGTGGGCGGAGTTGACCTCCGGACTTTGGGTTACTCCCAACAGCAACAAGAGAA
GATTAAACCTAAGGTGCGCTCAACAGTGGCGCAGCATCATGAGGCACTGGTTGGTCACGGGTTCACACATGCCCATA
TAGTAGCACTTTCACAACATCCCGCTGCACTCGGAACGGTTGCAGTAAAGTACCAAGACATGATTGCCGCATTGCCG
GAAGCAACTCATGAGGCCATTGTCGGAGTAGGCAAATACCACGGTGCTAGAGCTCTCGAAGCACTCCTCACTGTGGC
TGGCGAATTGAGGGGGCCCCCCCTTCAACTTGATACTGGACAGCTGTTGAAGATAGCGAAACGAGGGGGAGTAACCG
CAGTCGAGGCCGTCCACGCGTGGCGAAATGCATTGACGGGAGCGCCTCTCAACCTTACTCCAGATCAGGTTGTGGCC
ATAGCGAGCAACATCGGAGGTAAGCAAGCACTGGAAACTGTTCAACGATTGCTCCCAGTACTCTGCCAAGACCACGG
ATTGACCCCAGAACAGGTTGTGGCTATTGCCAGCAACGGCGGCGGTAAACAGGCTCTGGAAACTGTGCAGCGACTCT
TGCCGGTGTTGTGCCAAGCTCATGGCCTTACGCCAGACCAGGTTGTTGCTATCGCTAGCAACATCGGAGGCAAACAG
GCACTTGAAACGGTCCAACGCCTCTTGCCAGTATTGTGCCAGGCACATGGCCTCACCCCCGCCCAGGTTGTGGCCAT
CGCAAACAATAACGGGGGCAAGCAGGCTTTGGAGACAGTCCAACGATTGTTGCCGGTACTTTGTCAGGACCACGGGC
TGACCCCAGATCAAGTCGTAGCAATCGCTAGCAACATCGGCGGGAAGCAGGCCCTGGAAACGGTGCAGAGGCTCCTC
CCAGTGCTCTGCCAAGATCATGGCCTCACCCCAGAGCAAGTGGTCGCAATCGCAAACAATAACGGTGGCAAGCAAGC
CCTCGAAACTGTGCAAAGGTTGCTGCCAGTTCTCTGTCAAGCCCACGGTTTGACACCGGATCAGGTAGTGGCTATCG
CTAGCAACGGCGGTGGCAAGCAAGCTCTCGAGACAGTCCAGAGGCTTTTGCCAGTACTGTGCCAAGCGCACGGTCTC
ACTCCTGCGCAAGTTGTTGCTATAGCAAACAATAACGGTGGAAAGCAAGCTCTTGAGACTGTGCAGCGCCTGTTGCC
CGTCCTCTGTCAGGACCACGGTTTGACTCCAGACCAAGTTGTCGCCATTGCAAGCAACGGCGGTGGACGCCCTGCAC
TGGAGTCCATCGTCGCACAACTGTCTAGGCCGGACCCCGCCCTGGCTGCCCTTACTAATGATCACTTGGTTGCATTG
GCGTGTCTGGGCGGTCGGCCAGCGCTGGACGCAGTCAAGAAGGGACTCCCCCACGCCCCTGCACTTATAAAACGCAC
TAACAGGAGAATACCAGAACGGACGAGTCATCGAGTCGCTGGAGGGGGCGGTAGCGAAGTCCAGCTCCAAGAAAGCG
GCGGGGGCCTCGTGCAGCCAGGCGGGTCCCTGAGGCTGAGCTGTACCGCATCTGGAGTCACCATCTCTGCCCTTAAC
GCAATGGCTATGGGGTGGTATCGGCAGGCGCCCGGCGAGAGGAGAGTCATGGTAGCAGCCGTTAGCGAAAGGGGGAA
TGCGATGTACCGAGAGAGTGTTCAGGGACGATTTACTGTCACTCGGGATTTTACGAACAAAATGGTTTCATTGCAAA
TGGACAATTTGAAACCAGAGGACACCGCTGTTTACTACTGTCACGTCCTGGAAGATCGAGTAGATAGCTTCCATGAC
TATTGGGGCCAAGGAACACAAGTGACTGTCAGCTCC
SEQ ID NO: 887: ALFA tag inserted after the G66 residue relative to SEQ ID NO: 1 of MLT
with mutation: R333K_N335A_R336K_D416N substitutions relative to SEQ ID NO: 1
ATGCCGAAAAAAAAACGAAAGGTGTACCCCTACGATGTACCGGACTATGCAGGAAGCGCCCAGCACAGCGACTACCC
CGACGACGAGTTCAGAGCCGATAAGCTGAGTAACTACAGCTGCGACAGCGACCTGGAAAACGCCAGCACATCCGACG
AGGACAGCTCTGACGACGAGGTGATGGTGCGGCCCAGAACCCTGAGACGGAGAAGAATCAGCAGCTCTAGCAGCGAC
TCTGAATCCGACATCGAGGGCTCCAGACTGGAAGAGGAACTGAGAAGAAGGCTCACAGAAGGCCGGGAAGAGTGGAG
CCACGTGGACAACCCTCCTGTTCTGGAAGATTTTCTGGGCCATCAGGGCCTGAACACCGACGCCGTGATCAACAACA
TCGAGGATGCCGTGAAGCTGTTCATAGGAGATGATTTCTTTGAGTTCCTGGTCGAGGAATCCAACCGCTATTACAAC
CAGAATAGAAACAACTTCAAGCTGAGCAAGAAAAGCCTGAAGTGGAAGGACATCACCCCTCAGGAGATGAAAAAGTT
CCTGGGACTGATCGTTCTGATGGGACAGGTGCGGAAGGACAGAAGGGATGATTACTGGACAACCGAACCTTGGACCG
AGACCCCTTACTTTGGCAAGACCATGACCAGAGACAGATTCAGACAGATCTGGAAAGCCTGGCACTTCAACAACAAT
GCTGATATCGTGAACGAGTCTGATAGACTGTGTAAAGTGCGGCCAGTGTTGGATTACTTCGTGCCTAAGTTCATCAA
CATCTATAAGCCTCACCAGCAGCTGAGCCTGGATGAAGGCATCGTGCCCTGGCGGGGCAGACTGTTCTTCAGAGTGT
ACAATGCTGGCAAGATCGTCAAATACGGCATCCTGGTGCGCCTTCTGTGCGAGAGCGATACAGGCTACATCTGTAAT
ATGGAAATCTACTGCGGCGAGGGCAAAAGACTGCTGGAAACCATCCAGACCGTCGTTTCCCCTTATACCGACAGCTG
GTACCACATCTACATGGACAACTACTACAATTCTGTGGCCAACTGCGAGGCCCTGATGAAGAACAAGTTTAGAATCT
GCGGCACAATCAAGAAAGCCAAGGGCATCCCTAAGGACTTCCAGACCATCTCTCTGAAGAAGGGCGAAACCAAGTTC
ATCAGAAAGAACGACATCCTGCTCCAAGTGTGGCAGTCCAAGAAACCCGTGTACCTGATCAGCAGCATCCATAGCGC
CGAGATGGAAGAAAGCCAGAACATCGACAGAACAAGCAAGAAGAAGATCGTGAAGCCCAATGCTCTGATCGACTACA
ACAAGCACATGAAAGGCGTGGACCGGGCCAACCAGTACCTGTCTTATTACTCTATCCTGAGAAGAACAGTGAAATGG
ACCAAGAGACTGGCCATGTACATGATCAATTGCGCCCTGTTCAACAGCTACGCCGTGTACAAGTCCGTGCGACAAAG
AAAAATGGGATTCAAGATGTTCCTGAAGCAGACAGCCATCCACTGGCTGACAGACGACATTCCTGAGGACATGGACA
TTGTGCCAGATCTGCAACCTGTGCCCAGCACCTCTGGTATGAGAGCTAAGCCTCCCACCAGCGATCCTCCATGTAGA
CTGAGCATGGACATGCGGAAGCACACCCTGCAGGCCATCGTCGGCAGCGGCAAGAAGAAGAACATCCTTAGACGGTG
CAGGGTGTGCAGCGTGCACAAGCTGCGGAGCGAGACTCGGTACATGTGCAAGTTTTGCAACATTCCCCTGCACAAGG
GAGCCTGCTTCGAGAAGTACCACACCCTGAAGAATTAC
SEQ ID NO: 888: Ch6L left TALE fused to the recombinant variable domains of heavy-chain-
only antibodies (VHH) (NANOBODY) bound to an ALFA-tag peptide (NbALFA)
ATGGCGCCTAAGAAGAAGCGCAAGGTGGGCGGAGTTGACCTCCGGACTTTGGGTTACTCCCAACAGCAACAAGAGAA
GATTAAACCTAAGGTGCGCTCAACAGTGGCGCAGCATCATGAGGCACTGGTTGGTCACGGGTTCACACATGCCCATA
TAGTAGCACTTTCACAACATCCCGCTGCACTCGGAACGGTTGCAGTAAAGTACCAAGACATGATTGCCGCATTGCCG
GAAGCAACTCATGAGGCCATTGTCGGAGTAGGCAAATACCACGGTGCTAGAGCTCTCGAAGCACTCCTCACTGTGGC
TGGCGAATTGAGGGGGCCCCCCCTTCAACTTGATACTGGACAGCTGTTGAAGATAGCGAAACGAGGGGGAGTAACCG
CAGTCGAGGCCGTCCACGCGTGGCGAAATGCATTGACGGGAGCGCCTCTCAACCTTACGCCGGATCAAGTCGTGGCG
ATAGCTAGTAACGGAGGTGGTAAACAGGCGCTGGAGACGGTACAGCGCTTGCTGCCAGTGCTCTGCCAAGATCACGG
CCTTACGCCAGAGCAAGTGGTGGCTATTGCGAATAATAACGGTGGAAAACAGGCGTTGGAAACAGTCCAAAGACTGC
TCCCGGTGTTGTGCCAAGCTCATGGACTGACGCCCGATCAAGTAGTAGCTATCGCAAGCAACGGTGGGGGGAAACAG
GCACTGGAGACTGTCCAGAGGCTCCTCCCTGTGTTGTGCCAAGCCCATGGGCTTACTCCAGCTCAGGTTGTAGCGAT
CGCCAGCAACATTGGAGGAAAGCAGGCCCTGGAAACTGTTCAACGCCTCCTCCCAGTATTGTGCCAAGATCACGGAC
TTACCCCGGATCAAGTCGTCGCAATCGCGTCCCACGATGGCGGGAAACAGGCACTTGAGACCGTGCAACGACTGTTG
CCCGTGCTCTGCCAAGACCATGGCCTCACACCTGAGCAAGTAGTGGCGATTGCGTCTCACGATGGGGGCAAGCAGGC
GCTTGAGACCGTTCAGCGATTGCTTCCTGTCCTCTGCCAGGCCCACGGGTTGACACCTGATCAGGTGGTAGCCATTG
CGTCTAACGGTGGAGGTAAGCAAGCACTCGAGACCGTTCAAAGGCTCTTGCCAGTATTGTGTCAGGCTCATGGTCTT
ACGCCAGCCCAAGTTGTCGCCATCGCATCTCATGACGGTGGGAAGCAGGCACTTGAGACTGTACAAAGATTGCTCCC
AGTGTTGTGCCAGGACCACGGCTTGACGCCTGATCAAGTTGTAGCGATTGCTTCACATGATGGGGGCAGGCCTGCGT
TGGAGTCCATCGTCGCACAACTGTCTAGGCCGGACCCCGCCCTGGCTGCCCTTACTAATGATCACTTGGTTGCATTG
GCGTGTCTGGGCGGTCGGCCAGCGCTGGACGCAGTCAAGAAGGGACTCCCCCACGCCCCTGCACTTATAAAACGCAC
TAACAGGAGAATACCAGAACGGACGAGTCATCGAGTCGCTGGAGGGGGCGGTAGCGAAGTCCAGCTCCAAGAAAGCG
GCGGGGGCCTCGTGCAGCCAGGCGGGTCCCTGAGGCTGAGCTGTACCGCATCTGGAGTCACCATCTCTGCCCTTAAC
GCAATGGCTATGGGGTGGTATCGGCAGGCGCCCGGCGAGAGGAGAGTCATGGTAGCAGCCGTTAGCGAAAGGGGGAA
TGCGATGTACCGAGAGAGTGTTCAGGGACGATTTACTGTCACTCGGGATTTTACGAACAAAATGGTTTCATTGCAAA
TGGACAATTTGAAACCAGAGGACACCGCTGTTTACTACTGTCACGTCCTGGAAGATCGAGTAGATAGCTTCCATGAC
TATTGGGGCCAAGGAACACAAGTGACTGTCAGCTCC
SEQ ID NO: 889: Ch6R right TALE fused to to the recombinant variable domains of heavy-
chain-only antibodies (VHH) (NANOBODY) bound to an ALFA-tag peptide (NbALFA)
ATGGCGCCTAAGAAGAAGCGCAAGGTGGGCGGAGTTGACCTCCGGACTTTGGGTTACTCCCAACAGCAACAAGAGAA
GATTAAACCTAAGGTGCGCTCAACAGTGGCGCAGCATCATGAGGCACTGGTTGGTCACGGGTTCACACATGCCCATA
TAGTAGCACTTTCACAACATCCCGCTGCACTCGGAACGGTTGCAGTAAAGTACCAAGACATGATTGCCGCATTGCCG
GAAGCAACTCATGAGGCCATTGTCGGAGTAGGCAAATACCACGGTGCTAGAGCTCTCGAAGCACTCCTCACTGTGGC
TGGCGAATTGAGGGGGCCCCCCCTTCAACTTGATACTGGACAGCTGTTGAAGATAGCGAAACGAGGGGGAGTAACCG
CAGTCGAGGCCGTCCACGCGTGGCGAAATGCATTGACGGGAGCGCCTCTCAACCTTACACCTGATCAAGTAGTTGCC
ATTGCGAGTCATGATGGTGGGAAACAGGCCTTGGAAACTGTCCAGCGCCTTCTGCCTGTGCTTTGTCAAGATCATGG
GTTGACACCCGAGCAAGTTGTAGCTATTGCGTCACACGACGGTGGTAAACAGGCGCTGGAAACTGTTCAGCGCTTGC
TCCCAGTCCTCTGTCAGGCCCATGGTCTCACCCCGGACCAGGTAGTAGCTATAGCTAGCAATATCGGAGGAAAGCAA
GCGTTGGAAACCGTGCAGCGGTTGCTCCCCGTCCTGTGTCAAGCACACGGCTTGACGCCGGCCCAGGTGGTAGCCAT
TGCGTCTAACATCGGCGGCAAGCAGGCACTCGAGACTGTGCAACGGCTCCTGCCGGTTCTGTGCCAAGATCACGGTC
TTACTCCTGATCAGGTCGTTGCGATTGCTAGTAATGGCGGTGGAAAACAGGCCCTGGAGACTGTCCAGCGGCTTCTC
CCTGTCCTGTGCCAGGACCATGGACTCACACCAGAACAGGTAGTTGCAATAGCCTCTAATATAGGTGGCAAGCAAGC
ACTCGAGACAGTCCAAAGACTTCTTCCCGTCCTTTGCCAGGCGCATGGTCTCACCCCTGATCAAGTTGTTGCGATAG
CGAGTAACATTGGAGGAAAACAGGCATTGGAAACGGTACAACGGCTTCTCCCGGTCTTGTGTCAAGCGCATGGCCTC
ACCCCGGCCCAAGTGGTCGCTATAGCATCCAATATTGGGGGTAAGCAAGCACTGGAAACGGTTCAGCGACTTTTGCC
TGTGCTGTGCCAAGACCACGGACTTACACCGGACCAAGTTGTCGCTATCGCCTCCAACGGGGGAGGACGGCCGGCCT
TGGAGTCCATCGTCGCACAACTGTCTAGGCCGGACCCCGCCCTGGCTGCCCTTACTAATGATCACTTGGTTGCATTG
GCGTGTCTGGGCGGTCGGCCAGCGCTGGACGCAGTCAAGAAGGGACTCCCCCACGCCCCTGCACTTATAAAACGCAC
TAACAGGAGAATACCAGAACGGACGAGTCATCGAGTCGCTGGAGGGGGCGGTAGCGAAGTCCAGCTCCAAGAAAGCG
GCGGGGGCCTCGTGCAGCCAGGCGGGTCCCTGAGGCTGAGCTGTACCGCATCTGGAGTCACCATCTCTGCCCTTAAC
GCAATGGCTATGGGGTGGTATCGGCAGGCGCCCGGCGAGAGGAGAGTCATGGTAGCAGCCGTTAGCGAAAGGGGGAA
TGCGATGTACCGAGAGAGTGTTCAGGGACGATTTACTGTCACTCGGGATTTTACGAACAAAATGGTTTCATTGCAAA
TGGACAATTTGAAACCAGAGGACACCGCTGTTTACTACTGTCACGTCCTGGAAGATCGAGTAGATAGCTTCCATGAC
TATTGGGGCCAAGGAACACAAGTGACTGTCAGCTCC
SEQ ID NO: 890: Nucleotide sequence of non-CD19 donor element (4068 nucleotides)

| | |
|---|---|
| 1 | TTAACACTAC GGTGTCGGGT GAATTTCCCG CCAAAATATA AGATGGCGCG GGCAATTATT |
| 61 | TAGGTTTCCC GCGCAAAATC AGAGTTAGTT TAAAATGCCT ATATCTTTTG AAGTATGGGT |
| 121 | TCGATTGAAA TGAAATTTTC GGTTTCTTGT GTAAAAAATG AAAATTTATT CAATCACAGA |
| 181 | CATTTTTTTA TCTATTCAGT ACATAAATAA TTCTCGAGCT CCCCAGCATG CCTGCTATTG |
| 241 | TCTTCCCAAT CCTCCCCCTT TCCGCTCACA ATTCCACACA AAGCTTTAAA AGCCTTATAT |
| 301 | ATTCTTTTTT TTCTTATAAA ACTTAAAACC TTAGAGGCTA TTTAAGTTGC TGATTTATAT |
| 361 | TAATTTTATT GTTCAAACAT GAGAGCTTAG TACGTGAAAC ATGAGAGCTT AGTACGTTAG |
| 421 | CCATGAGAGC TTAGTACGTT AGCCATGAGG GTTTAGTTCG TTAAACATGA GAGCTTAGTA |
| 481 | CGTTAAACAT GAGAGCTTAG TACGTGAAAC ATGAGAGCTT AGTACGTACT ATCAACAGGT |
| 541 | TGAACTGCCA GATATCCCGC GTTGCTGGCG TTTTTCCATA GGCTCCGCCC CCCTGACGAG |
| 601 | CATCACAAAA ATCGACGCTC AAGTCAGAGG TGGCGAAACC CGACAGGACT ATAAAGATAC |
| 661 | CAGGCGTTTC CCCCTGGAAG CTCCCTCGTG CGCTCTCCTG TTCCGACCCT GCCGCTTACC |
| 721 | GGATACCTGT CCGCCTTTCT CCCTTCGGGA AGCGTGGCGC TTTCTCAATG CTCACGCTGT |
| 781 | AGGTATCTCA GTTCGGTGTA GGTCGTTCGC TCCAAGCTGG GCTGTGTGCA CGAACCCCCC |
| 841 | GTTCAGCCCG ACCGCTGCGC CTTATCCGGT AACTATCGTC TTGAGTCCAA CCCGGTAAGA |
| 901 | CACGACTTAT CGCCACTGGC AGCAGCCACT GGTAACAGGA TTAGCAGAGC GAGGTATGTA |
| 961 | GGCGGTGCTA CAGAGTTCTT GAAGTGGTGG CCTAACTACG GCTACACTAG AAGGACAGTA |
| 1021 | TTTGGTATCT GCGCTCTGCT GAAGCCAGTT ACCTTCGGAA AAAGAGTTGG TAGCTCTTGA |
| 1081 | TCCGGCAAAC AAACCACCGC TGGTAGCGGT GGTTTTTTTG TTTGCAAGCA GCAGATTACG |
| 1141 | CGCAGAAAAA AAGGATCTCA AGAAGATCCT TTGATCTTTT CTACGGGGTC TGACGCTCAG |
| 1201 | TGGAACGAAA ACTCACGTTA AGGGATTTTG GTCATGAGAT TATCAAAAAG GATCTTCACC |
| 1261 | TAGATCCTTT TAAATTAAAA ATGAAGTTTT AAATCAATCT AAAGTATATA TGAGTAAACT |
| 1321 | TGGTCTGACA GTTACCAATG CTTAATCAGT GAGGCACCTA TCTCAGCGAT CTGTCTATTT |
| 1381 | CGTTCATCCA TAGTTGCCTG ACTCCCCGTC GTGTAGATAA CTACGATACG GGAGGGCTTA |
| 1441 | CCATCTGGCC CCAGTGCTGC AATGATACCG CGAGACCCAC GCTCACCGGC TCCAGATTTA |
| 1501 | TCAGCAATAA ACCAGCCAGC CGGAAGGGCC GAGCGCAGAA GTGGTCCTGC AACTTTATCC |
| 1561 | GCCTCCATCC AGTCTATTAA TTGTTGCCGG GAAGCTAGAG TAAGTAGTTC GCCAGTTAAT |
| 1621 | AGTTTGCGCA ACGTTGTTGC CATTGCTACA GGCATCGTGG TGTCACGCTC GTCGTTTGGT |
| 1681 | ATGGCTTCAT TCAGCTCCGG TTCCCAACGA TCAAGGCGAG TTACATGATC CCCCATGTTG |
| 1741 | TGCAAAAAAG CGGTTAGCTC CTTCGGTCCT CCGATCGTTG TCAGAAGTAA GTTGGCTGCT |
| 1801 | GTATTATCAC TCATGGTTAT GGCAGCACTG CATAATTCTC TTACTGTCAT GCCATCCGTA |
| 1861 | AGATGCTTTT CTGTGACTGG TGAGTACTCA ACCAAGTCAT TCTGAGAATA GTGTATGCGG |
| 1921 | CGACCGAGTT GCTCTTGCCC GGCGTCAATA CGGGATAATA CCGCGCCACA TAGCAGAACT |
| 1981 | TTAAAAGTGC TCATCATTGG AAAACGTTCT TCGGGGCGAA AACTCTCAAG GATCTTACCG |
| 2041 | CTGTTGAGAT CCAGTTCGAT GTAACCCACT CGTGCACCCA ACTGATCTTC AGCATCTTTT |
| 2101 | ACTTTCACCA GCGTTTCTGG GTGAGCAAAA ACAGGAAGGC AAAATGCCGC AAAAAAGGGA |
| 2161 | ATAAGGGCGA CACGGAAATG TTGAATACTC ATACTCTTCC TTTTTCAATA TTATTGAAGC |
| 2221 | ATTTATCAGG GTTATTGTCT CATGAGCGGA TACATATTTG AATGTATTTA GAAAAATAAA |
| 2281 | CAAATAGGGG TTCCGCGCAC ATTTCCCCGA AAAGTGCCAC CTGACGTCGA CGGATCGGGA |
| 2341 | GATCTCCCGA TCCCCTATGG TCGACTCTCA GTACAATCTG CTCTGATGCC GCATAGTTAA |
| 2401 | GCCAGTATCT GCTCCCTGCT TGTGTGTTGG AGGTCGCTGA GTAGTGCGCG AGCAAAATTT |
| 2461 | AAGCTACAAC AAGGCAAGGC TTGACCGACA ATTGCATGAA GAATCTGCTT AGGGTTAGGC |
| 2521 | GTTTTGCGCT GCTTCGCGAT GTACGGGCCA GATATACGCG TTGACATTGA TTATTGACTA |
| 2581 | GTTATTAATA GTAATCAATT ACGGGGTCAT TAGTTCATAG CCCATATATG GAGTTCCGCG |
| 2641 | TTACATAACT TACGGTAAAT GGCCCGCCTG GCTGACCGCC CAACGACCCC CGCCCATTGA |
| 2701 | CGTCAATAAT GACGTATGTT CCCATAGTAA CGCCAATAGG GACTTTCCAT TGACGTCAAT |
| 2761 | GGGTGGAGTA TTTACGGTAA ACTGCCCACT TGGCAGTACA TCAAGTGTAT CATATGCCAA |
| 2821 | GTACGCCCCC TATTGACGTC AATGACGGTA AATGGCCCGC CTGGCATTAT GCCCAGTACA |
| 2881 | TGACCTTATG GGACTTTCCT ACTTGGCAGT ACATCTACGT ATTAGTCATC GCTATTACCA |
| 2941 | TGGTGATGCG GTTTTGGCAG TACATCAATG GGCGTGGATA GCGGTTTGAC TCACGGGGAT |
| 3001 | TTCCAAGTCT CCACCCCATT GACGTCAATG GGAGTTTGTT TTGGCACCAA AATCAACGGG |
| 3061 | ACTTTCCAAA ATGTCGTAAC AACTCCGCCC CATTGACGCA AATGGGCGGT AGGCGTGTAC |
| 3121 | GGTGGGAGGT CTATATAAGC AGAGCTCGTT TAGTGAACCG TCAGATCGCC TGGAGACGCC |
| 3181 | ATCCACGCTG TTTTGACCTC CATAGAAGAC ACCGGGACCG ATCCAGCCTC CGGACTCTAG |
| 3241 | AGGCGCCACC ATGGTGTCTA AGGGCGAAGA GCTCTTTACT GGCGTGGTGC CCATCCTGGT |
| 3301 | TGAATTGGAC GGAGATGTTA ACGGACACAA ATTTAGCGTA TCTGGAGAGG GCGAAGGTAA |
| 3361 | GTAATCGGTT CAAGTGATTC TCCTGCCTCA GCCTCCCAAG TAGCTGGGAT TAGAGGTCCC |
| 3421 | CACCACCATG CCTGGCTAAT TTTTTGTACT TTCAGTAGAA ATGGGGTTTT GCCATGTTGG |
| 3481 | CCAGGCTGTT CTCGAACTCC TGAGCTCAGG TGATCCAACT GTCTCGGCCT CCCAAAGTGC |
| 3541 | TGGGATTACA GGCGTGAGCC ACTGTGCCTA GCCTGAGCCA CCACGCCGGC CTAATTTTTA |
| 3601 | AATTTTTTGT AGAGACAGGG TCTCATTATG TTGCCCAGGG TGGTGTCAAG CTCCAGGTCT |
| 3661 | CAAGTGATCC CCCTACCTCC GCCTCCCAAA GTTGTGGGAT TGTAGGCATG AGCCACTGCA |
| 3721 | AGAAAACCTT AACTGCAGCC TAATAATTGT TTTCTTTGGG ATAACTTTTA AAGTACATTA |
| 3781 | AAAGACTATC AACTTAATTT CTGATCATAT TTTGTTGAAT AAAATAAGTA AAATGTCTTG |
| 3841 | TGAAACAAAA TGCTTTTTAA CATCCATATA AAGCTATCTA TATATAGCTA TCTATGTCTG |
| 3901 | GCGCGCCTAA CAACAAATAT TTGGTAATAC GCAGTTACAA TCGGCCCGAG TTTACACGTT |
| 3961 | TCCCGCACTC CACCGCAAAT CTATATGAAC CGAGTTAAGA CGGCTCCCGC GGAGTACGCG |
| 4021 | CAATTCACGC GAGCCGACTT AACTCGTTTC CCGCAATCCA AGTGTTAA |

SEQ ID NO: 891: Nucleotide sequence of CD19 CAR donor element (4330 nucleotides)

| | |
|---|---|
| 1 | TTAACACTTG GATTGCGGGA AACGAGTTAA GTCGGCTCGC GTGAATTGCG CGTACTCCGC |
| 61 | GGGAGCCGTC TTAACTCGGT TCATATAGAT TTGCGGTGGA GTGCGGGAAA CGTGTAAACT |
| 121 | CGGGCCGATT GTAACTGCGT ATTACCAAAT ATTTGTTGTC GACTCTCCCT ATAGTGAGTC |
| 181 | GTATTAATTC TCCCAGATCC TCTAGCGATA AGCTTGATAT CGAATTCGAG GTCGACTTCT |
| 241 | AGAGAGCTCA ATAAAAGAGC CCACAACCCC TCACTCGGCG GCTCCGGTGC CCGTCAGTGG |
| 301 | GCAGAGCGCA CATCGCCCAC AGTCCCCGAG AAGTTGGGGG GAGGGGTCGG CAATTGAACC |
| 361 | GGTGCCTAGA GAAGGTGGCG CGGGGTAAAC TGGGAAAGTG ATGTCGTGTA CTGGCTCCGC |
| 421 | CTTTTTCCCG AGGGTGGGGG AGAACCGTAT ATAAGTGCAG TAGTCGCCGT GAACGTTCTT |
| 481 | TTTCGCAACG GGTTTGCCGC CAGAACACAG GTAAGTGCCG TGTGTGGTTC CCGCGGGCCT |
| 541 | GGCCTCTTTA CGGGTTATGG CCCTTGCGTG CCTTGAATTA CTTCCACCTG GCTGCAGTAC |
| 601 | GTGATTCTTG ATCCCGAGCT TCGGGTTGGA AGTGGGTGGG AGAGTTCGAG GCCTTGCGCT |
| 661 | TAAGGAGCCC CTTCGCCTCG TGCTTGAGTT GAGGCCTGGC CTGGGCGCTG GGGCCGCCGC |
| 721 | GTGCGAATCT GGTGGCACCT TCGCGCCTGT CTCGCTGCTT TCGATAAGTC TCTAGCCATT |
| 781 | TAAAATTTTT GATGACCTGC TGCGACGCTT TTTTTCTGGC AAGATAGTCT TGTAAATGCG |
| 841 | GGCCAAGATC TGCACACTGG TATTTCGGTT TTTGGGGCCG CGGGCGGCGA CGGGGCCCGT |
| 901 | GCGTCCCAGC GCACATGTTC GGCGAGGCGG GGCCTGCGAG CGCGGCCACC GAGAATCGGA |
| 961 | CGGGGGTAGT CTCAAGCTGG CCGGCCTGCT CTGGTGCCTG GTCTCGCGCC GCCGTGTATC |
| 1021 | GCCCCGCCCT GGGCGGCAAG GCTGGCCCGG TCGGCACCAG TTGCGTGAGC GGAAAGATGG |
| 1081 | CCGCTTCCCG GCCCTGCTGC AGGGAGCTCA AAATGGAGGA CGCGGCGCTC GGGAGAGCGG |
| 1141 | GCGGGTGAGT CACCCACACA AAGGAAAAGG GCCTTTCCGT CCTCAGCCGT CGCTTCATGT |
| 1201 | GACTCCACGG AGTACCGGGC GCCGTCCAGG CACCTCGATT AGTTCTCGAG CTTTTGGAGT |
| 1261 | ACGTCGTCTT TAGGTTGGGG GGAGGGGTTT TATGCGATGG AGTTTCCCCA CACTGAGTGG |
| 1321 | GTGGAGACTG AAGTTAGGCC AGCTTGGCAC TTGATGTAAT TCTCCTTGGA ATTTGCCCTT |
| 1381 | TTTGAGTTTG GATCTTGGTT CATTCTCAAG CCTCAGACAG TGGTTCAAAG TTTTTTTCTT |
| 1441 | CCATTTCAGG TGTCGTGAGC CACCATGGCC TTACCAGTGA CCGCCTTGCT CCTGCCGCTG |
| 1501 | GCCTTGCTGC TCCACGCCGC CAGGCCGGAC ATCCAGATGA CACAGACTAC ATCCTCCCTG |
| 1561 | TCTGCCTCTC TGGGAGACAG AGTCACCATC AGTTGCAGGG CAAGTCAGGA CATCTCTAAG |
| 1621 | TATTTGAATT GGTATCAGCA GAAACCAGAT GGAACTGTTA AACTCCTGAT CTACCATACA |
| 1681 | TCAAGATTAC ACTCAGGAGT CCCATCAAGG TTCAGTGGCA GTGGGTCTGG AACAGATTAT |
| 1741 | TCTCTCACCA TTAGCAACCT GGAGCAAGAA GATATTGCCA CTTACTTTTG CCAACAGGGT |
| 1801 | AATACGCTTC CGTACACGTT CGGAGGGGGG ACTAAGTTGG AAATAACAGG CTCCACCTCT |
| 1861 | GGATCCGGCA AGCCCGGATC TGGCGAGGGA TCCACCAAGG GCGAGGTGAA ACTGCAGGAG |
| 1921 | TCAGGACCTG GCCTGGTGGC GCCCTCACAG AGCCTGTCCG TCACATGCAC TGTCTCAGGG |
| 1981 | GTCTCATTAC CCGACTATGG TGTAAGCTGG ATTCGCCAGC CTCCACGAAA GGGTCTGGAG |
| 2041 | TGGCTGGGAG TAATATGGGG TAGTGAAACC ACATACTATA ATTCAGCTCT CAAATCCAGA |
| 2101 | CTGACCATCA TCAAGGACAA CTCCAAGAGC CAAGTTTTCT TAAAAATGAA CAGTCTGCAA |
| 2161 | ACTGATGACA CAGCCATTTA CTACTGTGCC AAACATTATT ACTACGGTGG TAGCTATGCT |
| 2221 | ATGGACTACT GGGGTCAAGG AACCTCAGTC ACCGTCTCCT CAGCGGCCGC AGCGAAGCCC |
| 2281 | ACCACGACGC CAGCGCCGCG ACCACCAACA CCGGCGCCCA CCATCGCGTC GCAGCCCCTG |
| 2341 | TCCCTGCGCC CAGAGGCGTG CCGGCCAGCG GCGGGGGGCG CAGTGCACAC GAGGGGGCTG |
| 2401 | GACTTCGCCT GTGATATCTA CATCTGGGCG CCCTTGGCCG GGACTTGTGG GGTCCTTCTC |
| 2461 | CTGTCACTGG TTATCACCCT TTACTGCAAA CGGGGCAGAA AGAAACTCCT GTATATATTC |
| 2521 | AAACAACCAT TTATGAGACC AGTACAAACT ACTCAAGAGG AAGATGGCTG TAGCTGCCGA |
| 2581 | TTTCCAGAAG AAGAAGAAGG AGGATGTGAA CTGAGAGTGA AGTTCAGCAG GAGCGCAGAC |
| 2641 | GCCCCCGCGT ACCAGCAGGG CCAGAACCAG CTCTATAACG AGCTCAATCT AGGACGAAGA |
| 2701 | GAGGAGTACG ATGTTTTGGA CAAGAGACGT GGCCGGGACC CTGAGATGGG GGGAAAGCCG |
| 2761 | AGAAGGAAGA ACCCTCAGGA AGGCCTGTAC AATGAACTGC AGAAAGATAA GATGGCGGAG |
| 2821 | GCCTACAGTG AGATTGGGAT GAAAGGCGAG CGCCGGAGGG GCAAGGGGCA CGATGGCCTT |
| 2881 | TACCAGGGTC TCAGTACAGC CACCAAGGAC ACCTACGACG CCCTTCACAT GCAGGCCCTG |
| 2941 | CCCCCTCGCG AGGGCAGAGG CAGCCTGCTG ACATGTGGCG ACGTGGAAGA GAACCCTGGC |
| 3001 | CCCATGGTTG GTTCGCTAAA CTGCATCGTC GCTGTGTCCC AGAACATGGG CATCGGCAAG |
| 3061 | AACGGGGACT TCCCCTGGCC ACCGCTCAGG AATGAATCCA GATATTTCCA GAGAATGACC |
| 3121 | ACAACCTCTT CAGTAGAAGG TAAACAGAAT CTGGTGATTA TGGGTAAGAA GACCTGGTTC |
| 3181 | TCCATTCCTG AGAAGAATCG ACCTTTAAAG GGTAGAATTA ATTTAGTTCT CAGCAGAGAA |
| 3241 | CTCAAGGAAC CTCCACAAGG AGCTCATTTT CTTTCCAGAA GTCTAGATGA TGCCTTAAAA |
| 3301 | CTTACTGAAC AACCAGAATT AGCAAATAAA GTAGACATGG TCTGGATAGT TGGTGGCAGT |
| 3361 | TCTGTTTATA AGGAAGCCAT GAATCACCCA GGCCATCTTA AACTATTTGT GACAAGGATC |
| 3421 | ATGCAAGACT TTGAAAGTGA CACGTTTTTT CCAGAAATTG ATTTGGAGAA ATATAAACTT |
| 3481 | CTGCCAGAAT ACCCAGGTGT TCTCTCTGAT GTCCAGGAGG AGAAAGGCAT TAAGTACAAA |
| 3541 | TTTGAAGTAT ATGAGAAGAA TGATTAATCC TCAGGTGCAG GCTGCCTATC AGAAGGTGGT |
| 3601 | GGCTGGTGTG GCCAATGCCC TGGCTCACAA ATACCACTGA GATCTTTTTC CCTCTGCCAA |
| 3661 | AAATTATGGG GACATCATGA AGCCCCTTGA GCATCTGACT TCTGGCTAAT AAAGGAAATT |
| 3721 | TATTTTCATT GCAATAGTGT GTTGGAATTT TTTGTGTCTC TCACTCGGAA GGACATATGG |
| 3781 | GAGGGCAAAT CATTTAAAAC ATCAGAATGA GTATTTGGTT TAGAGTTTGG CAACATATGC |
| 3841 | CCATATGCTG GCTGCCATGA ACAAAGGTTG GCTATAAAGA GGTCATCAGT ATATGAAACA |
| 3901 | GCCCCCTGCT GTCCATTCCT TATTCCATAG AAAAGCCTTG ACTTGAGGTT AGATTTTTTT |
| 3961 | TATATTTTGT TTTGTGTTAT TTTTTTCTTT AACATCCCTA AAATTTTCCT TACATGTTTT |
| 4021 | ACTAGCCAGA TTTTTCCTCC TCTCCTGACT ACTCCCAGTC ATAGCTGTCC CTCTTCTCTT |
| 4081 | ATGGAGATCA TTTAGGTGAC ACTATAGAAG AGACTAGTAA TTATTTATGT ACTGAATAGA |
| 4141 | TAAAAAAATG TCTGTGATTG AATAAATTTT CATTTTTTAC ACAAGAAACC GAAAATTTCA |
| 4201 | TTTCAATCGA ACCCATACTT CAAAAGATAT AGGCATTTTA AACTAACTCT GATTTTGCGC |
| 4261 | GGGAAACCTA AATAATTGCC CGCGCCATCT TATATTTTGG CGGGAAATTC ACCCGACACC |
| 4321 | GTAGTGTTAA |

### EXAMPLES

Hereinafter, the present disclosure will be described in further detail with reference to examples. These examples are illustrative purposes only and are not to be construed to limit the scope of the present disclosure. In addition, various modifications and variations can be made without departing from the technical scope of the present disclosure.

### Example 1—Design of Transposon System

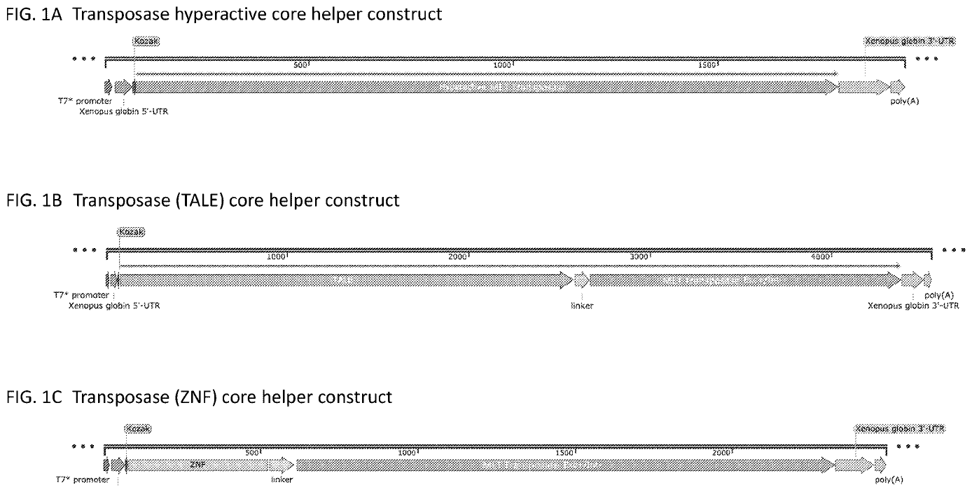

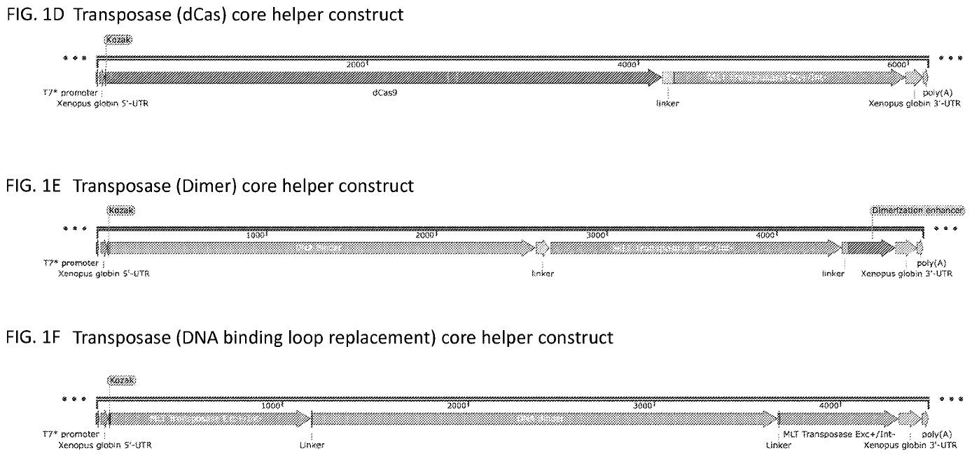

FIG. 1A-FIG. 1F depict six illustrative bioengineered RNA helper constructs that are contained in a replication backbone (e.g., plasmid or miniplasmid) with a T7 promoter (cap dependent), beta-globin 5′-UTR, and a helper enzyme with 2 or more mutations in the Myotis lucifugus helper followed by a beta-globin 3′-UTR, and a poly-alanine tail (FIG. 1A). TALEs, ZnF, or a dead Cas (dCas) binding protein (accompanied by guide RNAs) were linked to the N-terminus to target the specific TTAA sites at hROSA 26, AAVS1, chromosome 4, chromosome 22, and chromosome X loci. FIG. 1E depicts a construct with a dimerization enhancer. Without being bound to a particular theory, the dimerization enhancer may be selected from, without limitation, SH3, biotin, avidin, and rapamycin binders. The dimerization enhancer is replaced with an intein. FIG. 1F depicts a construct that interrupts the natural DNA binding loop present in MLT (Y281-T291 AND I332-P339) and renders the helper enzyme Exc+/Int−. The extrinsic DNA binder that is inserted in the DNA binding loop binds to a target that is within 9-25 bp from a site-specific TTAA in the genome.

### Example 2—Types of Covalent and Non-Covalent Linkers

This example shows the discovery of DNA binding proteins (e.g., without limitations, TALE and Cas9), linkers, and fusion sites that target specific TTAA.

FIG. 2A-FIG. 2B depict the types of covalent and non-covalent linkers that are used to directly fuse (i.e., link) protein sequence-specific DNA binding domains (e.g., without limitation, TALE, ZnF, dCas) that recognize target DNA sequences flanking the TTAA. In FIG. 2A, the arrow shows covalent linker that fuses DNA binders to the N-terminus of MLT transposase. The linkers are strings of amino acids of varying lengths and flexibility and include the original linker, AKLAGGAPAVGGGPKAADKFAATGGS (SEQ ID NO: 8). In FIG. 2B, the arrows show non-covalent linkers such as an antipeptide antibody (Ab) fused to a DNA binder and a peptide tag fused to the N-terminus or within the DNA binding loop (Y281-T291 and I332-P339) of MLT transposase. These components can be changed where the antipeptide Ab is fused to MLT transposase and the peptide tag is fused to the DNA binder.

FIG. 2A-FIG. 2B depict two different types of linkers used to bioengineer synthetic DNA binders and allow the flexibility to bind to nearby flanking recognition sites. The distance of the recognition site from the TTAA was determined empirically to be 9-25 bp using non-covalent and covalent (4× GGSGGSGGSGGS (SEQ ID NO: 878), original AKLAGGAPAVGGGPKAADKFAATGGS (SEQ ID NO: 8) linkers and internal loops insertions at Y281-T291 and I332-P339.

### Example 3—Design of Transposon System

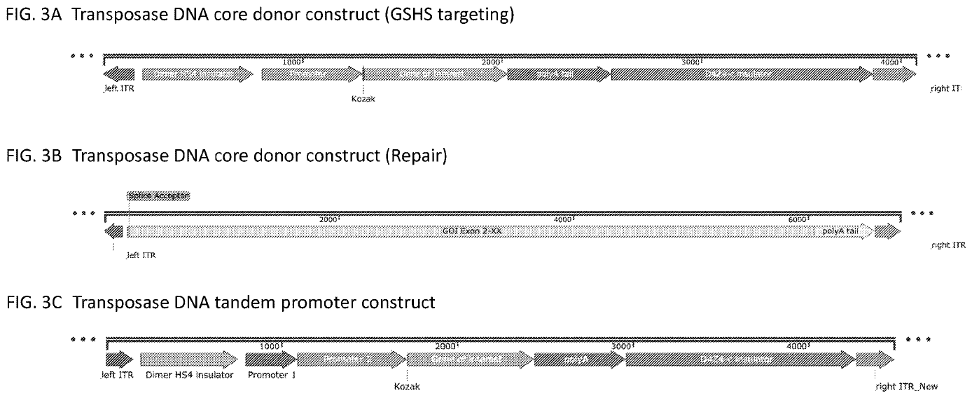

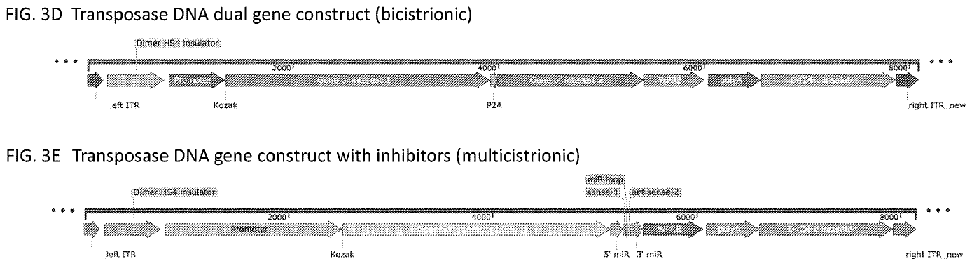

FIG. 3A depicts an illustrative core donor construct that is contained in a replication backbone (e.g., plasmid or miniplasmid) with a promoter driving a gene of interest (GOI) with a polyA tail flanked by two insulators and ITRs. The inverted terminal repeat (ITR) recognition sequences are included at the 5′- and 3′-ends. This construct is used for targeting genomic safe harbor sites (GSHS) or other loci.

FIG. 3B depicts an illustrative core donor construct that is contained in a replication backbone (e.g., plasmid or miniplasmid) with a splice acceptor site for exon 2 and other exons of a gene of interest (GOI) followed by a polyA tail and flanked by ITRs. The inverted terminal repeat (ITR) recognition sequences are included at the 5′- and 3′-ends. This construct is used for targeting endogenous genes in the first intron to repair downstream mutations.

FIG. 3C depicts an illustrative core donor construct that is contained in a replication backbone (e.g., plasmid or miniplasmid) with tandem promoters to affect expression in different tissues (e.g., without limitation, liver specific promoter, cardiac specific promoter) and a gene(s) of interest (GOI) followed by a polyA tail and flanked by ITRs. The inverted terminal repeat (ITR) recognition sequences are included at the 5′- and 3′-ends. This construct is used to differentially promote expression of genes in different organs, tissues or cell types.

FIG. 3D depicts an illustrative core donor construct that is contained in a replication backbone (e.g., plasmid or miniplasmid) with two or more genes of interest (GOI) linked by 2A “self-cleaving” peptides and followed by WPRE and a polyA tail. The construct is flanked by ITRs. The inverted terminal repeat (ITR) recognition sequences are included at the 5′- and 3′-ends. This construct is used for delivering multiple genes or genetic factors.

FIG. 3E depicts an illustrative core donor construct that is contained in a replication backbone (e.g., plasmid or miniplasmid) with a promoter(s) driving the expression of two or more genes as in FIG. 3D and linked to a sequence consisting of a 5′-miRNA, a sense and antisense miRNA pair, and completed with the 3′-miRNA. The construct is followed by WPRE and flanked by ITRs. The inverted terminal repeat (ITR) recognition sequences are included at the 5′- and 3′-ends. This construct combines protein replacement and miRNA to inhibit other related protein expression. The sense and anti-sense miRNA pair regulate the sense miRNAs, probably via modulating the chromatin architectures of the resided genomic loci. See Brown, T., Howe, F. S., Murray, S. C., Wouters, M., Lorenz, P., Seward, E., . . . . Mellor, J. (2018). Antisense transcription-dependent chromatin signature modulates sense transcript dynamics. Mol Syst Biol, 14(2), e8007; Murray, S. C., Haenni, S., Howe, F. S., Fischl, H., Chocian, K., Nair, A., & Mellor, J. (2015). Sense and antisense transcription are associated with distinct chromatin architectures across genes. Nucleic Acids Res, 43(16), 7823-7837.

### Example 4—Bioengineering the MLT Transposase Protein for Site-Specific Targeting and Hetrodimerizarion

FIG. 4A-FIG. 4C depict the concepts of bioengineering the MLT transposase protein of the present disclosure for site-specific targeting. As shown in FIG. 4A, the unengineered MLT transposase dimer binds the target DNA TTAA and flanking non-TTAA (nnnn) phosphodiester backbone (sequence independent). As shown in FIG. 4B, the recruitment to a site-specific TTAA is directed by fusing (i.e., linking) protein sequence-specific DNA binding domains that recognize target DNA sequences flanking the TTAA. Such DNA binding domains encompass, without limitation, TALE, ZnF, and dCas. In FIG. 4C, Exc+Int− mutations (depicted as “X” in the figure) in the intrinsic DNA binding domains decrease MLT transposase interactions with target DNA non-TTAA which flank the TTAA but leave excision and TTAA use intact (Exc+, Int−). This targeting strategy permits the insertion of a DNA element (GOI) at a single TTAA in a site-specific location in the genome.

### Example 5—Identification of Excision Positive and Integration Deficient Mutants

FIG. 5A-B depicts the results of integration and excision assays on mutants by amino acid residue. Number denotes the position of the amino acid residue relative to SEQ ID NO: 1. Plasmid DNA was isolated using the ZymoPURE II Plasmid Midiprep Kit. Plasmid DNA was isolated according to the manufacturer's protocol. To assess excision activity, helper plasmids were co-transfected into HEK293 cells with a donor plasmid with a DNA fragment comprising flanking recognition ends, inserted between two GFP coding sequences. Four days after transfection, cells were assessed for excision of the intervening DNA fragment and accompanying restoration of GFP expression. Briefly, HEK293 cells were seeded in 12-well plates at a density of 2.0×10E5 cells per well. The following day, cells were co-transfected with a GFP donor plasmid and the various SalioGase helper plasmids at a 3:1 ratio (900 ng of donor plasmid: 300 ng of helper plasmid). Transfections were performed in triplicate using X-tremeGENE9 DNA transfection reagent at a 1:3 ratio (1.2 μg of DNA: 3.6 μl transfection reagent per well). Four days after transfection, cells were harvested and assessed for GFP expression via FACS. To assess integration activity, helper plasmids were co-transfected into HEK293 cells with a donor plasmid expressing GFP. Cells were assessed for GFP expression seventeen days post transfection. Briefly, HEK293 cells were seeded in 12-well plates at a density of 2.0×10E5 cells per well. The following day, cells were co-transfected with a GFP donor plasmid and the various SalioGase helper plasmids at a 1:1 ratio (600 ng of donor plasmid: 600 ng of helper plasmid). Transfections were performed in triplicate using X-tremeGENE9 DNA transfection reagent at a 1:3 ratio (1.2 μg of DNA: 3.6 μL transfection reagent per well). On day 2 post transfection, half of the cells were harvested and stained using the Live/Dead fixable FarRed kit. Live cells were assessed for GFP expression via FACS. The remaining cells were carried for 17 days post-transfection and split twice per week. On day 17 post-transfection, cells were again harvested and stained using the Live/Dead fixable FarRed kit. Live cells were assessed for GFP expression via FACS. Integration efficiency was calculated using the following formula: Integration efficiency=(D17% GFP)/(D2% GFP). Excision positive (EXC+) and integration deficient (INT−) mutants are shown in FIGS. 5A and 5B. FIG. 5A shows three integration deficient ranges were analyzed for selection of top excision-only candidates: variants with integration activity less than the negative control (0-1.25%, 6 candidates), variants with integration activity 1×-2× greater than the negative control (1.25-2.5%), 5 candidates, and variants with integration activity 2×-4× greater than the negative control (2.5-5.0%, 2 candidates). FIG. 5B shows full-length helpers in an MLT mutant background (S8P/C13R/D416N) Mutations were made to five amino acids hypothesized to contact DNA in combinations of single, double and triple mutations. One helper was not complete and was excluded from the dataset (marked by an an arrow). Excision activity is shown in black (first bar) and integration activity is shown in gray (second smaller bar). Each experimental condition was run in duplicate.

### Example 6—Identification Deletion Mutants and Fusion Protein Mutants

FIG. 6 depicts helpers with a N-terminus fusion of a zinc finger (E2C) by the original linker on the MLT mutants background (S8P/C13R/D416N) and Exc+Int− mutations. Excision activity was spared despite the addition of the N terminus zinc finger.

The graph shows all full-length helpers in MLT three mutant background (S8P/C13R/D416N) and additional mutations were fused to the E2C zinc finger and analyzed for baseline excision (first black bars) and integration (second gray bars) activity. Mutations were made to five amino acids hypothesized to contact DNA in combinations of single, double and triple mutations. One helper was not complete and was excluded from the dataset (marked by arrow). All experimental conditions were run in duplicate.

### Example 7—A 5-Step Plasmid Landing Pad Assay in HEK293 Cells to Identify Site-Specific Targeting Using MLT N-Terminus Zinc Finger Fusions with Exc+Int− Mutants

This example demonstrates, inter alia, the development of landing pad assay in HEK293 and show site- and sequence-specific targeting.

FIG. 7 depicts a 5-step plasmid landing pad assay in HEK293 cells to identify site-specific targeting using MLT transposase or other mobile elements (e.g., without limitation, recombinases, integrases, transposases).

Step 1 involves transfection of HEK293 cells using a donor DNA with CMV driving the 5′-half (left) of GFP followed by a splice-donor (SD) site, MLT transposase fusion helpers with various linkers and DNA binding fusions linked to the N-terminus of MLT transposase, and a plasmid landing pad (reporter plasmid) with site specific DNA binding recognition sites flanking a TTAA followed by a splice acceptor site (SA) and the 3′-half (right) half of GFP.

Step 2 shows the mechanism of splicing and integration into the landing pad after transfection.

In Step 3, the left and right halves of GFP are joined and the SA and SD are spliced out thus turning on GFP (GFP readout).

Step 4 is the PCR amplification step to identify targeting.

Step 5 uses Amplicon-Seq to analyze integration in specific sequence regions.

FIG. 7 depicts plasmid cell-based assay to assess integration patterns. Step 1 to Step 3 involves transfection of HEK293 cells using a donor plasmid, reporter plasmid, and bioengineered MLT transposase. The integration readout is GFP expression by splicing the 5′-left GFP region to the 3′-right GFP region. Step 4 and Step 5 uses PCR and sequencing to analyze integrants. The DNA is extracted and the insertions or amplified using oligonucleotide primers within donor insert and outside the landing pad. Briefly the cell pellets are prepared for lysis using Viagen DirectCell according to manufacturer's protocol. Proteinase K powder (0.4 mg/ml) and 90 μl of buffer is added to each pellet and rotated for 3 hrs at 55° C. The mixture is heat inactivated for 45 min at 85° C. and 1.0 μl of lysate is used as a genomic DNA template. 1 μl of lysis was used for genomic PCR template. Forward (outside landing pad) and reverse primers (within insert) with barcodes are added to a 20 μl master mix in a 20 μl reaction containing 10 μl KOD ONE BLUE, 7.8 ul water and 0.6 μl each primer (10 μM). The PCR mixture is hot started at 95° C. for 30 seconds followed by 32 PCR cycles (denaturation 95° C. for 10 seconds, annealing at 60° C. for 5 seconds, and extension for 68° C. for 5 seconds). Plasmid cell-based assay was used to assess integration patterns. Step 5 uses Amplicon-Seq to analyze integration in specific sequence regions. The ultra-deep sequencing of PCR products (amplicons) used oligonucleotide barcodes designed to capture the regions of interest, followed by next-generation sequencing (NGS). Briefly, the remaining 11 ul of the PCR reaction is cleaned using the Zymo DNA Clean & Concentrator, according to manufacturer's protocol. The DNA is quantified and diluted to 20 ng/ul and samples with unique barcodes are mixed in equal amounts and analyzed by NGS. The bioinformatic output by internal amplicon seq analysis software shows the flanking sequence, position on reporter, number of reads, percent insertion at each TTAA site.

### Example 8—PCR Amplification Identifies Targeting by Exc+Int− MLT N-terminus Znf Fusions

FIG. 8A-FIG. 8B depict PCR amplification to identify targeting Step 4 in FIG. 7. In FIG. 8A, a landing pad with no DNA binding recognition sites [zinc fingers (ZnF) in this case, but could be TALE, Cas, etc.] is used as a negative control. Landing pads with DNA binding recognition sites (ZnF in this case, but could be TALE, Cas, etc.) on both sides of the target TTAA are analyzed for targeting. In FIG. 8B, a 2% agarose gel shows a molecular ladder (lane 1) and the the PCR products using no ZnF fused to the N-terminus of MLT S8P/C13R (Lane 2 negative control) compared to lane 3 MLT with an N-terminus ZnF fusion where a band at 450 bp shows targeting at the TTAA flanked by the zinc fingers as shown on FIG. 8A. In subsequent lanes transposases with S8P/C13R/D416N mutations that are black bolded have Exc+Int− mutations but no N-terminus ZnF. Lanes with transposases with S8P/C13R/D416N mutations that are gray bolded have both Exc+Int− mutations and N-terminus ZnF fusions. The unique 450 bp PCR products in the lanes in gray shows targeting at the TTAA flanked by the zinc fingers as shown on FIG. 8A. The following ZnF-fusion Exc+Int− MLT transposases S8P/C13R/D416N mutants show on target activity: S8P/C13R/D416N/R333K/N335A/R336K, S8P/C13R/D416N/R333S/K334A/N335A, S8P/C13R/D416N/Y281A, S8P/C13R/D416N/K334A, S8P/C13R/D416N/K334A/N335A, S8P/C13R/D416N/K334A/N336A, S8P/C13R/D416N/N335A, S8P/C13R/D416N/N335A/R336A, S8P/C13R/D416N/R333A, S8P/C13R/D416N/R333A/K334A, S8P/C13R/D416N/R333A/K335A, S8P/C13R/D416N/R333A/K336A, S8P/C13R/D416N/R336A, S8P/C13R/D416N/Y281A/K334A, S8P/C13R/D416N/Y281A/K334A/N335A, E2C/S8P/C13R/D416N/K334A, E2C/S8P/C13R/D416N/K334A/N335A, E2C/S8P/C13R/D416N/K334A/R336A, E2C/S8P/C13R/D416N/N335A, E2C/S8P/C13R/D416N/N335A/R336A, E2C/S8P/C13R/D416N/R333A, E2C/S8P/C13R/D416N/R333A/K334A, E2C/S8P/C13R/D416N/R333A/K335A, E2C/S8P/C13R/D416N/R333A/K336A, E2C/S8P/C13R/D416N/R336A, E2C/S8P/C13R/D416N/R281A, E2C/S8P/C13R/D416N/Y281A/K334A, E2C/S8P/C13R/D416N/Y281A/K334A/N335A, or E2C/S8P/C13R/D416N/Y281A/N335A.

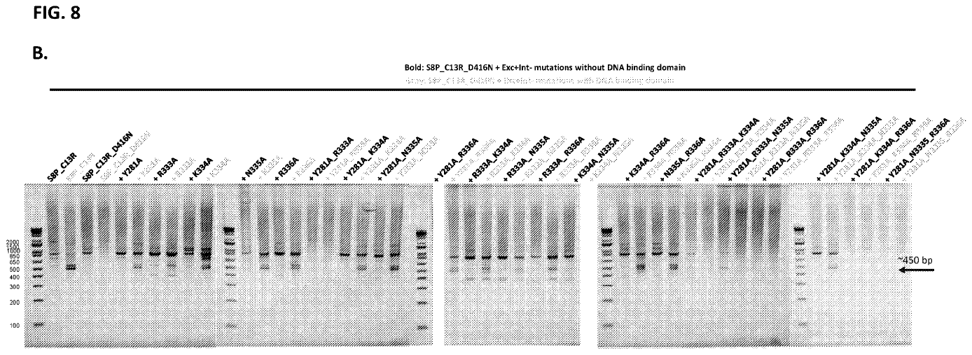

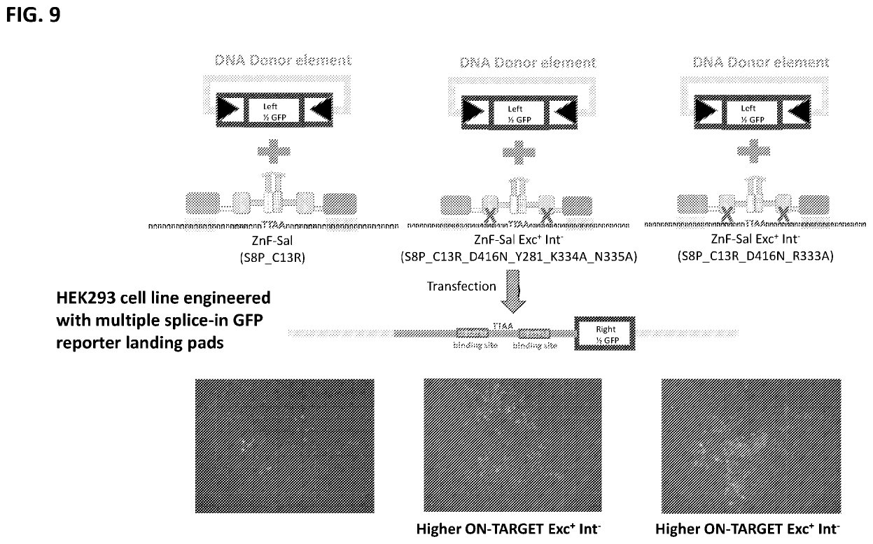

FIG. 9 depicts the results of targeting using a donor element that splices into an artificial landing pad in HEK293 cells. The landing pads were integrated at TA dinucleotides within the human genome using the transpoase, Sleeping Beauty (SB100X). Multiple landing pads containing a splice-in GFP reporter site 5′ to the target TTAA were integrated in the genome. These cell lines were used to test the ability of helper enzymes fused with ZnFs to recognize the binding sites. Helper fusions without ZnF fusions and ZnF helper fusions with natural DNA binders present (ZnF-Sal S8P/C13R) showed little compared integration to two helper enzymes with Exc+Int− mutations (S8P/C13R/D416N/Y281/K334A/N335A, S8P/C13R/D416N/R333A). Twenty-one days after transfection, fluorescent GFP imaging shows more cells expressed GFP when transfected with ZnF-fusion helpers with Exc+Int− mutations. Without being bound to a particular theory, the results show that Exc+Int− mutants provide site-specific integration in the human genome.

### Example 9: Target-Specific Insertion of Transgenes in Human Cells Using a Modular Transposase

To develop a targetable transposable elements (TE) system, it was reasoned, without wishing to be bound by theory, that off-target insertions would be minimized by disabling contacts with the genomic DNA via the native single transpoases enzyme (Ts) DNA binding domain (DBD). Upon adding artificial DNA binders such as a programmable ZnF, TALE, or Cas9, the Ts would be directed to the target genome sequence and integration activity would be rescued.

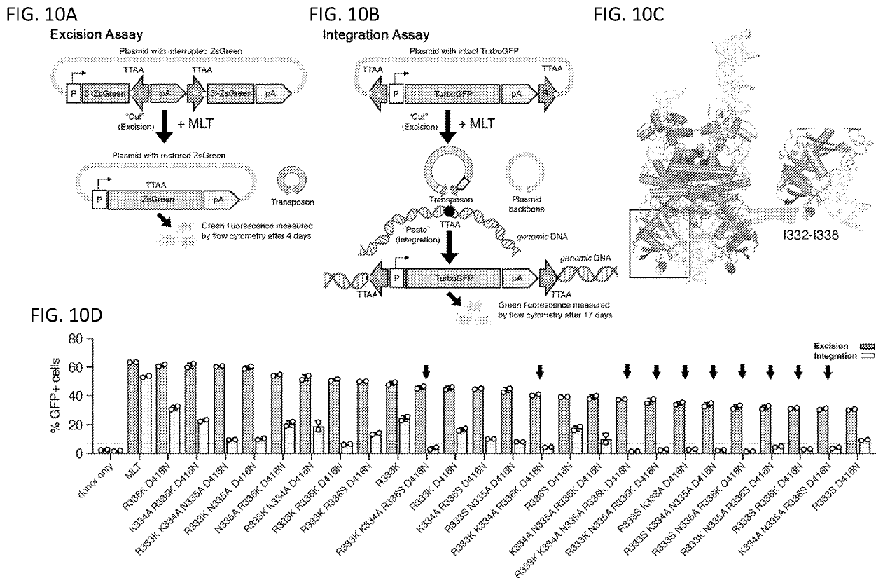

The initial experiments focused on generating a hyperactive MLT with high levels of both integration and excision. Previous experiments showed that phosphorylation of the N-terminal domain of the related Ts, piggyBac (pB), decreases transposition. Using an integration assay, wildtype MLT was compared to an MLT N-terminal variant surrounding a putative casein kinase II phosphorylation site and observed a 2-fold increase in Ts activity (S8P, C13R). These mutations were included in all future experiments. To identify potential mutable MLT domains that would reduce integration but maintain enzymatic catalysis and excision activity (Exc+Int−), MLT structural data predicted by AlphaFold was used. Excision and integration assays were developed to screen for these desired Exc+Int− mutants. The excision assay utilized a ZsGreen gene cassette disrupted by an MLT transposon (Tn) within the coding sequence (FIG. 10A). Upon expression of the Ts and seamless excision of the Tn, the ZsGreen gene was reformed, resulting in expression as measured by flow cytometry. The integration assay featured a plasmid containing a Tn with a constitutively active TurboGFP (FIG. 10B). Co-transfection with a MLT Ts plasmid resulted in Tn integration. A green signal was measured by flow cytometry after 17 days to dilute residual Tn plasmid signals.

To reduce off-target integration, alanine scanning of residues along a loop (I332-I338) predicted by AlphaFold to interact with the target DNA was analyzed (FIG. 10C). Substitutions I332A, R333A, R336A, G337A, I338A strongly impaired both integration and excision, while substitutions K334A and N335A slightly reduced these activities (FIG. 14A). Given the direct interactions of the positively charged arginine residues R333 and R336 with the target DNA, a series of mutants spanning positions R333-R336 were generated, with residues R333 and R336 either left unchanged or converted to lysine or serine. The residues K334A and N335A were left unchanged or replaced with an alanine, totaling 32 unique mutations. Because mutations in the putative target DNA-interacting loop invariably reduced excision activity, the MLT D416N mutation was included, without wishing to be bound by theory, as it was structurally homologous to the pB mutant, D450N, a mutant known to improve excision activity in the context of integration-dampening mutations. D416N was combined with the previous set of 32 mutations (n=64) and showed that 10 improved excision activity to greater than 30% while keeping integration efficiency less that 5% (FIG. 10D and FIG. 14B).

One of the mutants, containing R333K, K334A, N335A, R336K and D416N (herein referred to as m1), exhibited a 1.4% GFP signal in the integration assay. This low untargeted integration rate resembled the ‘background’ signal of the negative control with no Ts helper. Therefore, m1 was further characterized.

### Linking Artificial DNA Binders to MLT Targets Integration to Plasmids in Human Cells

To determine the design parameters for a sequence-specific Ts, a scaffold with the well-characterized ZnF, E2C, tethered to the Ts was engineered. It was reasoned, without being bound to a particular theory, that a three-finger ZnF with a small footprint (~90 amino acids) would offer improved targeting efficiency while minimally interfering with transpososome formation.

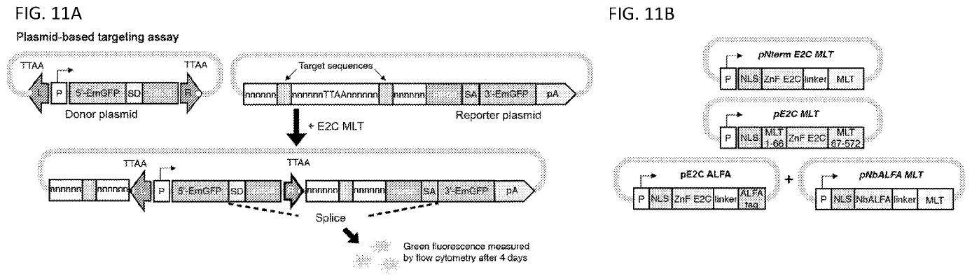

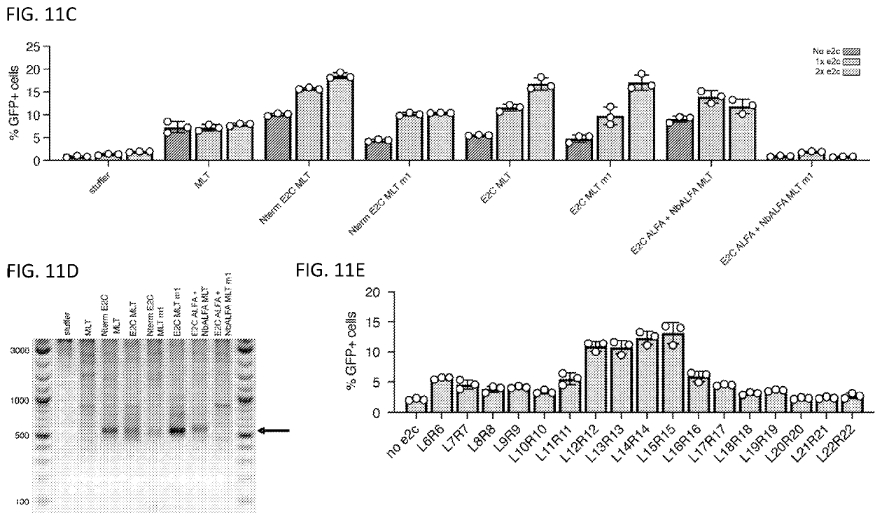

A two-plasmid targeting assay (plasmid-to-plasmid, P2P) was developed to screen different Ts combinations in a high-throughput manner. The first was a donor plasmid in which the Tn contained a CMV promoter driving the first 5′-half of an EmGFP gene followed by a splice donor sequence. The second was a reporter plasmid, in which the target sequence is followed by a splice acceptor and the 3′ fragment of EmGFP (FIG. 11A). Successful excision of the Tn followed by insertion near the target sequence resulted in restored EmGFP expression that was measured by flow cytometry. PCR primers were used to amplify the Tn-reporter junction and identify the location of insertions. In the initial reporter design, a 639 bp target ‘region’ containing 26 TTAA sites and the e2c recognition target sequence to study targeting performance in a localized region of competing native integration sites was included. A companion control reporter plasmid used for measuring ‘untargeted’ integration, contained the identical target region but lacked the e2c recognition target sequence.

Initial experiments explored three primary Ts frameworks (FIG. 11B): (i) “N term E2C MLT” with E2C fused to the N-terminus (covalent approach), (ii) “E2C MLT” with E2C inserted after the glycine at amino acid 66 within the N-terminus, (iii) “E2C ALFA+NbALFA MLT” with E2C tethered to MLT through recombinant variable domains of heavy-chain-only antibodies (VHH) (NANOBODY) bound to an ALFA-tag peptide.

The unstructured N-terminal domain may serve as a de facto ‘flexible linker’ for covalently linked DNA binders. It was reasoned, without being bound to a particular theory, that inserting the ZnF binder internally within the N-terminal domain would effectively shorten this linker and may position the catalytic domain closer the target DNA. The non-covalent strategy aimed to link the DNA binder to the Ts via a transient interaction, potentially allowing them to separate and undergo necessary conformational changes during catalytic insertion.

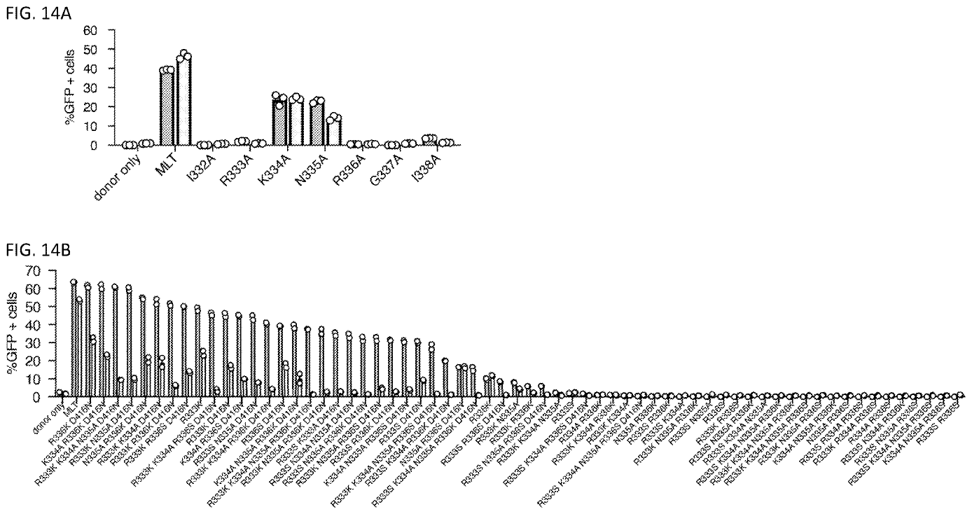

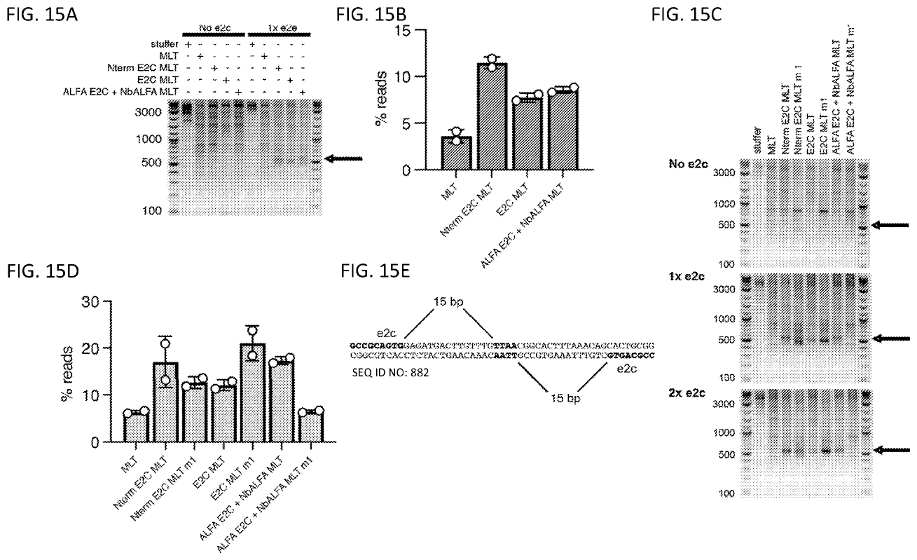

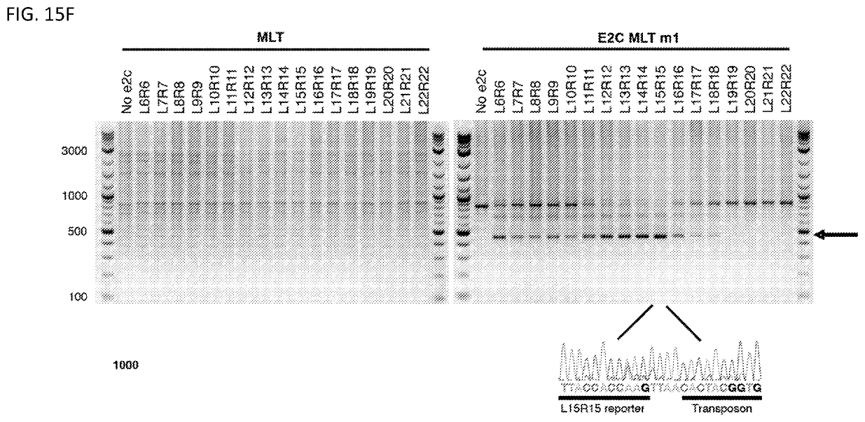

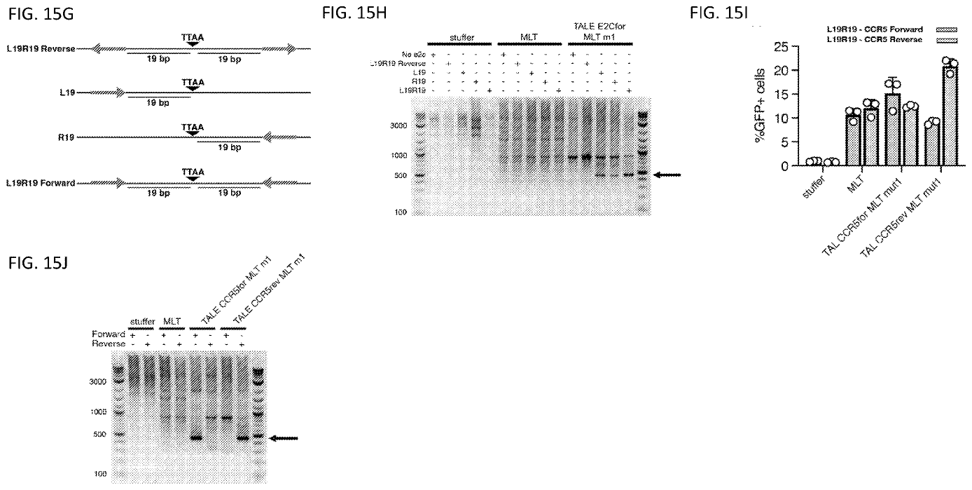

PCR analysis of integration products spanning the Tn-target sequence junction demonstrated that all three strategies linking MLT to E2C resulted in prominent PCR products at the expected sizes for insertion near the e2c recognition sequence (FIG. 15A). Amplicon sequencing revealed that insertions primarily occurred into a TTAA site 15 bp downstream of the e2c recognition sequence.

Assuming this was the preferred orientation for our engineered enzyme, it was hypothesized that targeting efficiency would improve when two e2c recognition sequences flanked a central TTAA. To test this, a reporter with e2c recognition sites positioned 15 bp on either side of the targeted TTAA was designed. Using the different linking strategies, different combinations were tested using the m1 MLT Exc+Int− mutant selected from previous experiments. The reporters with the dual e2c recognition sites showed increased targeting efficiency by flow cytometry and brighter PCR products of the expected size compared to reporters with one or no e2C recognition sites. The m1 MLT Exc+Int− mutant with an internal E2C binding protein fused at the MLT G66 N-terminal residue (E2C MLT m1), displayed an especially prominent PCR product at the expected target site (FIG. 11C, FIG. 11D, and FIGS. 15B-15D). To determine the spacing between the DNA recognition site and the TTAA target site, reporters with e2c recognition sequences at varying distances (6 to 22 bp) up- and downstream from the TTAA target site were synthesized. All other TTAAs within a 100 bp window of the target site were eliminated to reduce confounding effects. Consistent with previous results, the spacing for E2C MLT m1 was 14- or 15-bp (FIG. 11E, FIG. 15E, and FIG. 15F). To test if this ZnF-based strategy could target endogenous human sites, loci with identical 9 bp target sequences spaced either 14 or 15 bp from a central TTAA were identified. 29 ZnFs were synthesized by modular assembly and cloned into the MLT G66 residue of Exc+Int− MLT m1. Site-specific endogenous genomic targeting was not detectable by droplet digital PCR (ddPCR). The lack of targeting may be due to the inherent difficulties in engineering high-affinity ZnF proteins to user-defined targets. Therefore, TALEs were tested as alternate DNA binders.

To test TALEs in the system, a framework that was mutated to remove the requirement of a 5′T was used, generating two TALEs to the forward and reverse complement of the e2c recognition sequence. Each TALE was inserted at the MLT G66 residue in the Exc+Int− mutant (TAL E2C forward MLT m1 and TAL E2C reverse MLT m1). P2P assay was repeated with reporters containing flanking e2c target sequences spaced 14-22 bp from a central TTAA. Improved targeting at 18 or 19 bp for the forward TALE but no targeting using the reverse TALE (FIG. 15F-15H) was observed. As with ZnFs, targeting was improved by the presence of flanking e2c recognition sites. As further confirmation of the compatibility and versatility of TALEs in the system, forward (TAL CCR5forward) and reverse complement (TAL CCR5reverse) TALEs flanking a TTAA target site at the CCR5 locus were designed (FIGS. 15I-15J). These TALEs were placed in the MLT m1 scaffold and generated reporters with the target sequences appropriately spaced and oriented. As expected, it was observed that there was targeting at 18 or 19 bp for the forward TALE but no targeting using the reverse TALE (FIG. 15J).

### The Target TTAA Flanking Sequence Influences the Insertion Choice by Exc+Int− Mutants

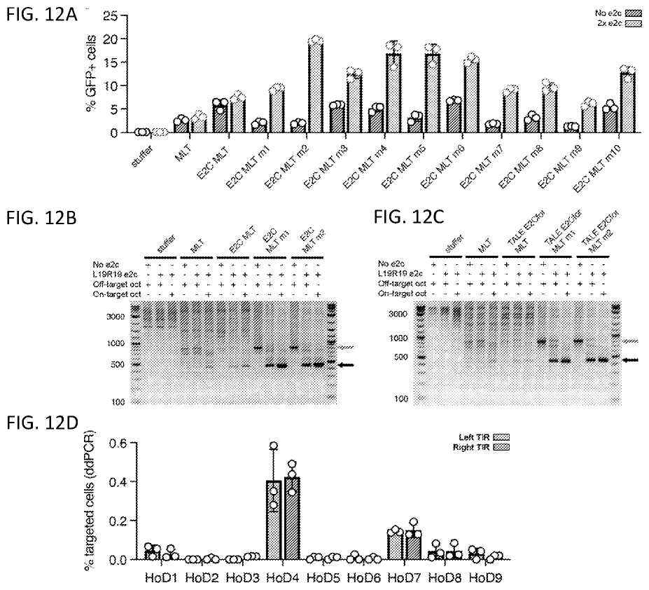

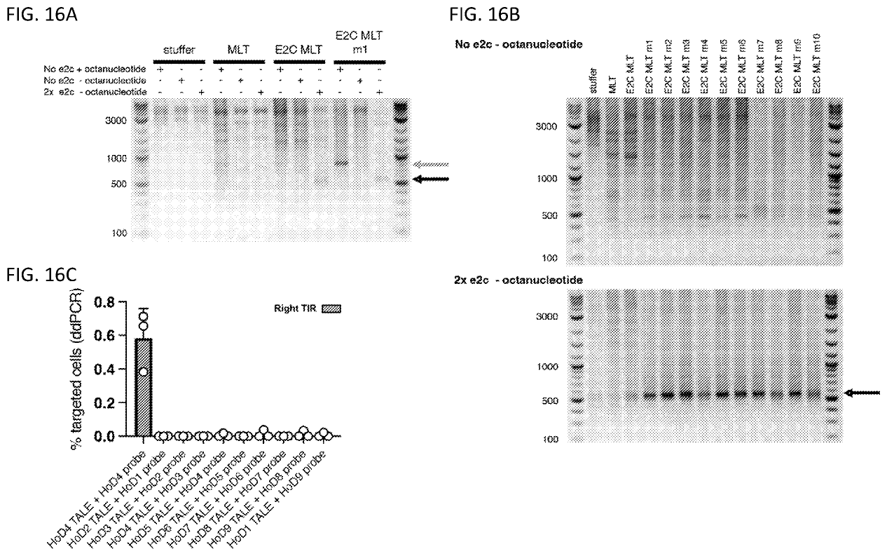

In all P2P assays, reproducible PCR products, representing untargeted TTAAs were observed. These off-targets were attributed to the presence of preferential TTAA integration sites within the reporter plasmid. For example, the Exc+Int− mutant m1 consistently inserted the Tn at a specific off-target TTAA site (shown at 843 bp in gels, FIG. 11D, FIG. 15C, FIG. 15D, FIG. 15H, and FIG. 15J). Sequencing showed that this specific TTAA site was flanked by TA dinucleotides (taTTAAta). Integration events at this site were in direct competition with the target TTAA. ‘TA’ pyrimidine-purine base steps were known to confer greater intrinsic DNA bending, which could be related to MLT Exc+Int− mutants, due to their impaired native DNA binding, which may have a stronger preference for more deformable TTAA target sequences such as the taTTAAta octanucleotide observed here. Interestingly, pB required a tight bend in the target TTAA to facilitate integration. The off-target taTTAAta octanucleotide sequence was deleted and it improved the sensitivity of the plasmid-based targeting assay (FIG. 16A). Nine additional Exc+Int− mutants (Table 18, E2C MLT m2-m10) were tested with the revised reporter plasmid and observed a 2-fold increase in targeting by the R333K, N335A, R336K, D416N (m2) compared to m1 (FIG. 12A). To validate that the taTTAAta octanucleotide was a preferential integrating site, this revised reporter plasmid and cloned two e2c recognition sequences spaced at 15 bp flanking the single octanucleotide target site was used. Experiments using this reporter were compared to other reporters. The MLT mutant (E2C MLT m2) showed preferential targeting at the taTTAAta octanucleotide (FIG. 12B). Similar results were obtained using a TALE in place of the E2C ZnF (FIG. 12C). These results guided the design of experiments targeting the human genome.

TABLE 18
E2C_MLT m2-m10

| Name | Mutations |
|---|---|
| m1 | R333K_K334A_N335A_R336K_D416N |
| m2 | R333K_N335A_R336K_D416N |
| m3 | R333K_K334A_R336S_D416N |
| m4 | R333S_K334A_D416N |
| m5 | R333K_K334A_R336K_D416N |
| m6 | R333S_K334A_N335A_D416N |
| m7 | R333S_N335A_R336K_D416N |
| m8 | R333K_N335A_R336S_D416N |
| m9 | R333S_R336K_D416N |
| m10 | K334A_N335A_R336S_D416N |

### Covalent Fusion of a TALE to MLT Targets Octanucleotide Sites in the Human Genome

To investigate the nucleotide preferences of Exc+Int− mutant in a genomic context, genomic integration using MLT without DNA binders were performed and identified integration sites using a modified self-reporting transposon (SRT) protocol. Insertions within ttTTAAaa and the taTTAAta sites were used and genomic sites containing identical 9 bp recognition sequences for homodimeric DNA binding proteins spaced 18 or 19 bp flanking the target octanucleotide site were chosen. These homodimeric flanking sites (HoD1-9) were selected to enable the use of a single TALE MLT helper instead of two. As an additional filter, only target sites that were >50 kb from coding genes or transcription start sites were selected. Six taTTAAta (HoD1-6) and three ttTTAAaa (HoD7-9) sites were chosen as candidate sites (FIG. 16B). TALEs designed to bind the flanking recognition sequences of these sites were placed after the MLT G66 residue in the MLT m2 mutant. Ste-specific genomic insertions in up to 0.8% of cells by ddPCR in HEK293T cells were detected, using probe and primer combinations for detection of insertions from both the left and right terminal inverted repeats (TIR) (FIG. 12D). In control experiments where a Ts was targeted to an octanucleotide site different from the one being queried by ddPCR, background levels of signal were observed, suggesting that targeting was TALE-mediated and not due to non-specific but preferential integration into octanucleotide sites. These results suggested that TALEs direct Tn insertions in the human genome at detectable levels without selection.

### Non-Covalent Linkage of TALEs to MLT Improves Targeting Efficiency to the Human Genome

Using results from covalent fusion of a TALE after the MLT G66 residue, experiments improving efficiency by using a non-covalent approach were conducted. To functionally test this hypothesis, the 13 amino acid ALFA tag were inserted after the G66 residue in the MLT m2 mutant (ALFA MLT). A separate vector with a TALE fused to recombinant variable domains of heavy-chain-only antibodies (VHH) (NANOBODY) against the ALFA tag (TALE NbALFA) was made.

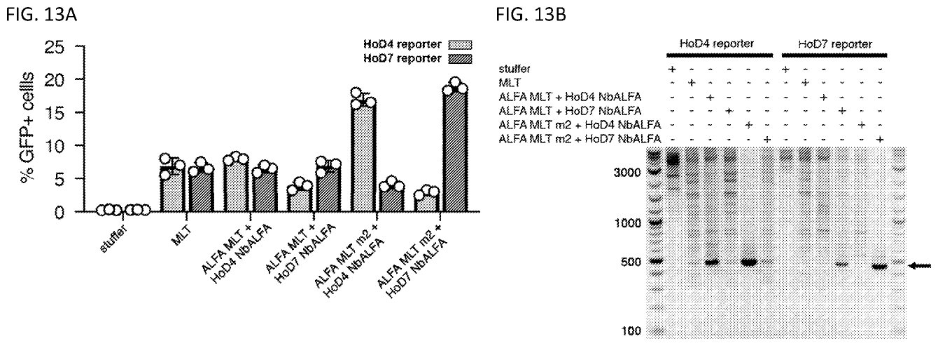

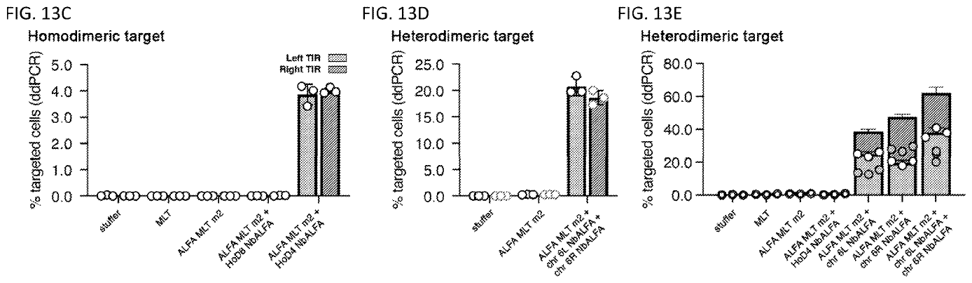

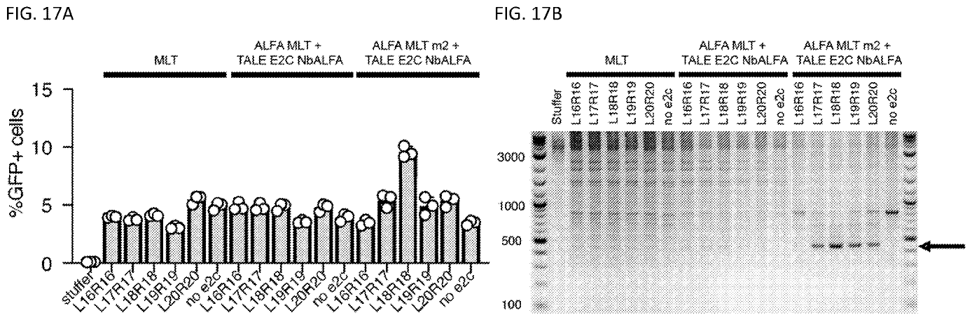

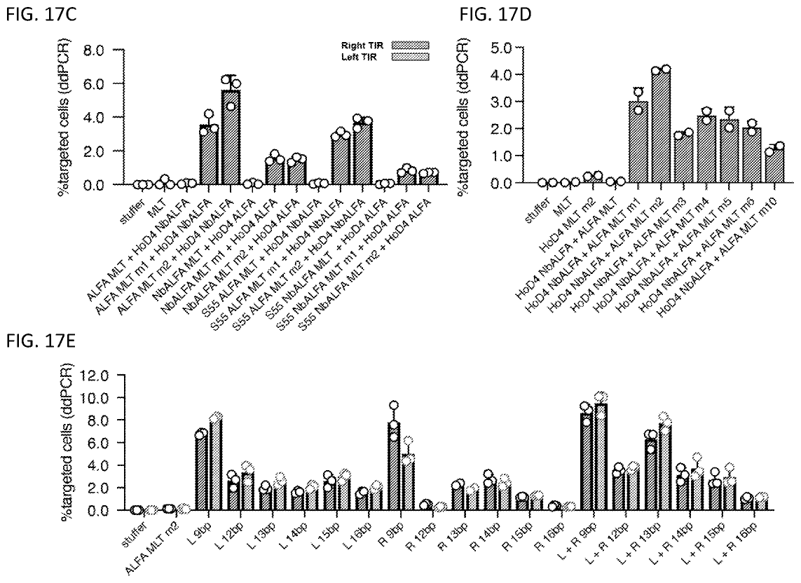

Using the P2P assay, it was found that spacing between the TALE recognition site and target TTAA was 18 bp, like the covalent strategy using the same TALE (FIG. 17A and FIG. 17B). TALE NbALFA fusion proteins directed to the top two sites identified by the covalent approach were constructed, HoD4 and HoD7 and observed efficient targeting for TALEs matched to their target sequences but not for mismatched controls (FIG. 13A and FIG. 13B).

To assess the non-covalent strategy in a genomic context, TALE NbALFA fusion proteins targeted to the three loci amenable to targeting in the covalent experiments was used. A 7.9% targeting efficiency at the HoD4 site was reached, a ~10-fold improvement over the covalent system (FIG. 13C). Targeting to the HoD7 and HoD8 sites also improved, albeit to a lesser degree. Notably, the octanucleotide sequences differed between HoD4 (taTTAAta), HoD7, and HoD8 (ttTTAAaa). Control mismatched TALE and probe experiments did not produce a signal. To explore further engineering, the NbALFA with the ALFA tag on the TALE and on MLT, MLT m1, and MLT m2 were swapped. These domains after the MLT G66 and the MLT S55 residues were inserted. Although it was expected the longer linker between the TALE and the structured portion of MLT to improve targeting, these variants did not outperform the combination HoD4 NbAlfa+ALFA MLT m2 (FIG. 17C). In addition, like the covalently linked DNA binders, other Exc+Int− mutants did not improve efficiency (FIG. 17D).

### Heterodimeric TALEs Flanking MLT Hotspots Mediate Genomic Targeting

Initial engineering used TTAA targets with identical flanking recognition sequences for homodimeric DNA binding proteins to avoid potential confounding effects due to differential binding affinities. To expand the number of genomic loci available for integration and improve targeting specificity, the restraint inherent in homodimeric targeting were removed and chose octanucleotide target sites that were: (i) recovered in all twelve replicates of the SRT assay, (ii) an octanucleotide site preferentially targeted by MLT, (iii) in open chromatin, and (iv) in a genomic safe harbor site. Based on these criteria, a ttTTAAaa target site on chromosome (Chr) 6 that was >120 kb from the nearest gene was chosen. Two TALE NbALFA fusions designed to bind different recognition sequences spaced 18 bp on either side of the target were generated. Using these heterodimeric TALEs (Chr6L NbALFA and Chr6R NbALFA) a 39.4% targeting efficiency in unselected HEK293T cells was achieved (FIG. 13D). Testing TALEs with longer binding sites did not elicit improved targeting compared to 9 bp TALEs (FIG. 17E). Sorting for successfully transfected cells, integration increased to 62.8%. Single TALE transfections with either left or right TALEs resulted in 38.1% and 24.7% efficiencies, respectively, indicating that the contribution of each TALE was additive (FIG. 13E).

### Transposon-Mediated Non-Viral CAR-T Therapy was Effective at Treating Human Leukemia and Lymphoma Cells

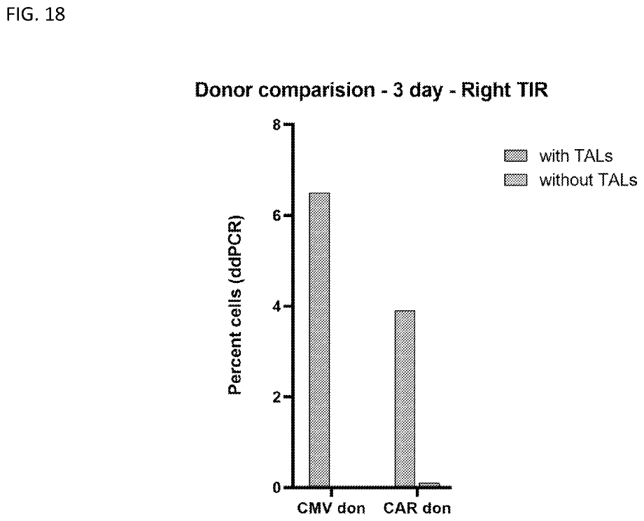

Ex vivo efficacy studies were conducted in T cells from three healthy human donors that were transposed with CD-19 CAR inserted at a chromosome 6 safe harbor locus (FIG. 18). FIG. 18 depicts ddPCR results using the non-covalent strategy linking flanking left and right TALEs to MLT m2 for use in generating CAR-T cells expressing CD19 at the chromosome 6 genomic safe harbor site. ddPCR was performed using a forward primer and probe in the genome and a reverse primer on the right terminal inverted repeats (TIR) of the Tn. All ddPCR values were normalized using the product reference ratio (PRR). A donor without CAR19 (SEQ ID NO: 890) showed about 6.5% of cells with targeting using a single right TIR primer (i.e., haploid genome). A donor with CAR19 (SEQ ID NO: 891) showed 4% targeting using a single right TIR primer (i.e., haploid genome). Transposition produced favorable CD4 and CD8 T-cell ratios, memory phenotype and exhaustion phenotypes, cytotoxicity cells against CD19-negative K562 (CML) and CD19-positive Nalm6 (ALL), Daudi and Raji (both Burkitt lymphoma) and release of proinflammatory cytokines IFNγ, TNFα, and lytic granule protease GZMB. The transgene on- and off-target integration profile was like HEK293T cells.

### Materials and Methods

### Plasmid Construction

All plasmids were generated via Gibson cloning using the NEBuilder HiFi DNA Assembly reagent (New England Biolabs, NEB). Plasmid DNA was isolated using the ZymoPURE Plasmid Miniprep Kit (Zymo) according to the manufacturer's protocol.

### Transposase (Helper) Plasmids

To generate the MLT helper plasmid, the gene for the full-length Myotis lucifugus transposase (MLT) was codon-optimized for human expression and synthesized as a gBlock gene fragment (Integrated DNA Technologies, IDT) and cloned into pcDNA3.3-TOPO. Insertion of the ALFA tag at position G66 was performed using specified primers and the Gibson cloning methodology. Fusion domains, including the E2C ZnF, the recombinant variable domains of heavy-chain-only antibodies (VHH) (NANOBODY) bound to an ALFA-tag peptide (NbALFA), and all transcription activator-like effectors (TALEs) targeted to 9 bp sequences, were synthesized as gBlocks and cloned into the vector via Gibson cloning. TALEs targeted to >12 bp sequences were generated using the Voytas Lab-Golden Gate TALEN and TAL Effector Kit 2.0 (Addgene).

### Excision Plasmids

To prepare CAG MLT exGFP, a gBlock was synthesized containing the ZsGreen gene interrupted at a naturally occurring TTAA site by a 607 bp Tn, which contains the left and right TIRs flanking the BGH pA. The interrupted gene was placed under the CAG promoter. Upon excision of the Tn, the naturally occurring TTAA site in the ZsGreen gene is reconstructed following seamless repair, restoring the original ZsGreen sequence.

### Integration Plasmids

To construct the MLT GIN donor, the terminal inverted repeat (TIR) sequences from the pBB GIN donor were swapped with MLT TIR sequences that were synthesized as gBlocks and cloned by Gibson assembly.

### Plasmid Reporter Assay Components

Donor and reporter plasmids were modified from the previously described EmGFP splicing based reconstitution assay. To prepare the EmGFP exon1 donor plasmid, the 5′-half of EmGFP and SMN1 intron was PCR amplified from 1/2 EmGFP SMN1 transposon and placed under the CMV promoter. The donor Tn was flanked by the TIR sequences of MLT that were synthesized as gBlocks. To create the reporter plasmids, the 3′-half of EmGFP was PCR amplified from pT4 SMN1 2/2 emGFP and cloned downstream of a 639 bp target region containing 26 TTAA sites. Reporter plasmids contained unique DNA binder recognition sequences. No e2c did not contain a target sequence. 1×e2c and 2×e2c contained a single or two e2c sequences placed 15 bp away from the target TTAA, respectively. Reporters containing endogenous sequences contained 86 bp of genomic sequence inserted into the target region. For experiments interrogating the required spacing of target sequences to the TTAA, alternate TTAA sites within 100 bp of the single target TTAA were removed and e2c sequences were inserted flanking the target TTAA at an indicated spacing on either side.

### Mammalian Cell Culture

### General Culture Conditions

HEK293T cells (Thermo Fisher Scientific) were maintained in Dulbecco's Modified Eagle Medium (DMEM) with GlutaMAX (Gibco) supplemented with 10% heat inactivated fetal bovine serum FBS (Neuromics), 1× Penicillin/Streptomycin (Gibco), and 0.75 μg/ml Amphotericin B (Gibco). Cells were grown at 37° C. with 5% carbon dioxide (CO₂) and media was replenished every 2 days.

### Excision Assay

HEK293T cells were seeded in 12-well plates at a density of 2.5×10⁵ cells per well. The following day, cells were co-transfected with the CAG MLT exGFP donor plasmid and a MLT helper plasmid at a 3:1 ratio (900 ng of donor plasmid: 300 ng of helper plasmid). CAG MLT exGFP contained a CAG promoter driving a ZsGreen gene that was interrupted by a MLT Tn. Following excision of the Tn by the Ts encoded on the helper plasmid, the ZsGreen gene is reformed leading to expression of the ZsGreen fluorescent protein. Transfections were performed using the X-tremeGENE9 DNA transfection reagent (Roche) at a 1:3 ratio (1.2 μg of DNA: 3.6 μl transfection reagent per well). Following four days of culture, percent ZsGreen+ cells was measured by flow cytometry using an Attune NXT flow cytometer (Thermo Fisher Scientific).

### Integration Assay

Like the excision assay, HEK293T cells were seeded in 12-well plates. The following day, cells were co-transfected with the MLT GIN donor plasmid and a MLT helper plasmid at a 1:1 ratio (600 ng of donor plasmid: 600 ng of helper plasmid). MLT GIN contained a CMV promoter driving a TurboGFP gene. Ts capable of integration transferred the Tn from the donor plasmid to the genome. Following 17 days of culture, transient expression was lost and the remaining TurboGFP+ cells contained genomic integrations. Transfections were performed using the X-tremeGENE9 DNA transfection reagent (Roche) at a 1:3 ratio (1.2 μg of DNA: 3.6 μl transfection reagent per well). Following two days of culture, half the cells were resuspended and the percentage of TurboGFP+ cells was measured by flow cytometry to assess transient transfection efficiency. The remaining cells were cultured until 17 days post transfection, and the percentage of TurboGFP+ cells were again measured by flow cytometry. Integration efficiency was calculated using the following formula: Integration efficiency=(D17% GFP)/(D2% GFP).

### Plasmid-Based Targeting Assay

HEK293T cells were seeded in 24-well plates at a density of 1.25×10⁵ cells per well. The following day, cells were co-transfected with 300 ng of helper plasmid, 150 ng of EmGFP exon1 donor plasmid and 150 ng of reporter plasmid. The EmGFP exon1 donor plasmid contained the CMV promoter driving the 5′-half (exon1) of EmGFP followed by a splice-donor (SD). The reporter plasmid contained the target TTAA site, including the indicated flanking DNA binder recognition sequences, followed by a splice acceptor (SA) and the 3′-half (exon2) of EmGFP. Successful targeting of the Tn by the Ts, encoded by the helper plasmid, resulted in alignment of the two halves of EmGFP (exon1+exon2) and subsequent splicing of the sequence between the SD and SA resulted in expression of the EmGFP fluorescent protein. Transfections were performed using TransIT-2020 (Mirus Bio) at a 1:3 ratio (0.6 μg of DNA: 1.8 μl transfection reagent per well). Following three days of culture, half the cells were pelleted and the remaining cells were measured by flow cytometry. To prepare PCR template, cell pellets were lysed with 90 μl DirectPCR Cell lysis reagent (Viagen) with 1.0 mg/mL proteinase K (Meridian Bioscience) and rotated in a hybridization oven for three hours at 55° C., then heat-inactivated for 45 min at 85° C. PCR was performed using 1 μl of lysate, 10 μl of KOD ONE BLUE polymerase, 7.8 μl water and 0.6 μl each primer (10 μM). The forward primer was located on the reporter plasmid and the reverse primer (with or without amplicon sequencing barcodes) was located on the donor Tn. PCR cycle conditions were 95° C. for 30 sec followed by 32 cycles of 98° C. for 10 sec, 60° C. for 5 sec, and 68° C. for 10 sec. Products were run on a 2% agarose gel in Tris-acetate-EDTA buffer and fragments for Sanger sequencing were extracted using the Zymo Gel DNA recovery Kit (Zymo). For amplicon sequencing, half the PCR reaction was cleaned using the DNA Clean and Concentrator Kit (Zymo) and eluted barcoded products were pooled in equimolar amounts and submitted for sequencing (Genewiz). Amplicon sequence reads were analyzed with custom Python scripts (available upon request) to generate percent reads containing inserted Tn at designated reporter plasmid TTAA sites.

### Genome Integration Assay

HEK293T cells were seeded in 24-well plates and the following day, cells were co-transfected with 100 ng of helper plasmid and 500 ng of EmGFP exon1 donor plasmid. Transfections were performed using TransIT-2020 (Mirus Bio) at a 1:3 ratio (0.6 μg of DNA: 1.8 μl transfection reagent per well). Following three days of culture, the cells were pelleted and lysed (described above) to prepare template for ddPCR.

Droplet Digital PCR (ddPCR) ddPCR was performed on genomic DNA (gDNA) to determine the frequency of targeted integrations. Reactions (25 μl) contained 25 ng gDNA, 12.5 μl of 2×ddPCR Supermix for Probes, 5 units XhoI (NEB), target and reference primers at 900 nM and probes at 250 nM. The plate was sealed with a PX1 PCR Plate Sealer (BioRad). Droplets were prepared using the QX200 AutoDG Droplet Generator System (BioRad). PCR cycle conditions were 95° C. for 10 min followed by 45 cycles of 95° C. for 30 sec, 60° C. for two min and 72° C. for two min, and a final incubation at 98° C. for 10 min. Droplets were read on a QX200 Droplet Reader (BioRad) and analyzed using QX Manager 1.2 Standard Edition software (BioRad).

### Whole Genome Integration Distribution Analyses by a Self-Reporting Transposon (SRT)

Previously published methods for preparing SRT libraries were adapted and performed sequencing using iSeq 100 and NextSeq 2000 Illumina platforms. The methodology involved using RNA-seq with calling cards and a MLT SRT donor by removing the polyadenylation signal (PAS) downstream of the reporter gene in the transposon. RNA polymerase II (Pol II) was used to transcribe the SRT reporter, continuing through the terminal inverted repeat (TIR) into the flanking genomic sequence. Thus, the SRTs “self-report” their locations via the unique genomic sequence present in the 3′ untranslated regions (UTRs) of the reporter gene transcripts. These SRT-derived transcripts contained the transposon-genome junction and permitted the insertions to be mapped.

### Bioinformatic Algorithm for Identifying Genomic Insertion Sites and Flanking Sequence Patterns

For the SRT calling card pipeline, a modified bioinformatic algorithm as described previously was used. Unlike conventional approaches utilizing single cell barcoding, the method directly analyzed the RNA data, allowing for multi-mapped reads up to 10 sites and retaining the sequence TTAA during trimming, without discarding integrations at non-TTAA sites. For each sample, the 150×2 reads were demultiplexed using i7 and i5 barcodes, where i5 was inline in read1. Cutadapt was used for trimming the reads, removing adapters, and extracting the inline i5 barcode and the left ITR sequence. The R1 sequences were aligned to the human hg38 reference genome using STAR with default parameters. Using BAM alignment, the location of each R1 was tagged by their barcode and position in the reference genome using a custom Python script adapted from previously published methods. Genecode and Homer annotation were employed to identify gene and intergenic locations of the insertion sites. Sequences ranging from 4 to 8 base pairs surrounding each insertion site were extracted to collect sequence motifs.

Without limitation in the present Examples, an engineered mammalian-derived transposase that precisely integrates DNA in human cells at efficiencies of up to 39.4% of unselected transfected cells was created. The gene delivery system operates without inducing unprotected DSBs and exhibited negligible off-target integrations compared to wildtype transposases. Through strategic mutations within putative target DNA-interacting residues and the incorporation of TALE DNA binding proteins, site-specific DNA insertion by a TE-based system was enabled. The experiment used a well-defined recombinant variable domains of heavy-chain-only antibodies (VHH) (NANOBODY)-tag linkage in which the recombinant variable domains of heavy-chain-only antibodies (VHH) (NANOBODY) bound a site embedded within the flexible N-terminal region of the transposase. Without being bound to a particular theory, a DNA binder non-covalently associated with an internally embedded tag outperformed direct N-terminal fusions, as well as arrangements where the DNA binder itself was inserted within the transposase polypeptide chain. Without being bound to a particular theory, this example achieved the highest targeting efficiency using TALE DNA-binding proteins containing only 8.5 repeats.

The example used a pre-screening strategy to identify genomic sites that would potentially be targetable with the engineered enzyme. Nine target sites with identical recognition sequences for homodimeric DNA binding proteins were screened and achieved 7.9% target integration at a site (HoD4) selected simply based on the presence of a taTTAAta octanucleotide. Sites with different flanking recognition sequences for heterodimeric DNA binding proteins offer an expanded target sequence choice which allowed the selection to include sites found in natural ‘hotspots’ that were informed by SRT analysis. Additional filters were applied including the requirement of a MLT preferred octanucleotide target site that resided in open chromatin and within a genomic safe harbor region. Targeting efficiency to a chromosome 6 safe harbor hotspot reached almost 60% of cells.

### EQUIVALENTS

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features herein set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior disclosure.

As used herein, all headings are simply for organization and are not intended to limit the disclosure in any manner. The content of any individual section may be equally applicable to all sections.

What is claimed is:

1. A composition comprising a helper enzyme or a nucleic acid encoding the helper enzyme comprising:
an amino acid sequence having at least about 80% sequence identity to SEQ ID NO: 1 and a non-polar aliphatic amino acid at position 2 of SEQ ID NO: 1, or a position corresponding thereto,
one or more amino acid substitutions selected from S8X, C13X, and D416X of SEQ ID NO: 1 or a substitution at a position corresponding thereto, and
one or more amino acid substitutions selected from R333X, K334X, N335X, R336X, Y281X, C282X, G283X, E284X, G285X, K286X, R287X, L288X, L289X, E290X, T291X, I332X, G337X, I338X, and P339X of SEQ ID NO: 1 or a substitution at a position corresponding thereto.

2. A composition comprising (a) a helper enzyme or a nucleic acid encoding the helper enzyme and (b) a targeting element or a nucleic acid encoding the targeting element, wherein:
the helper enzyme comprises an amino acid sequence having at least about 80% sequence identity to SEQ ID NO: 1 and has a non-polar aliphatic amino acid at position 2 of SEQ ID NO: 1 or a substitution at a position corresponding thereto,
one or more amino acid substitutions selected from S8X, C13X, and D416X of SEQ ID NO: 1 or a substitution at a position corresponding thereto, and
one or more amino acid substitutions selected from R333X, K334X, N335X, R336X, Y281X, C282X, G283X, E284X, G285X, K286X, R287X, L288X, L289X, E290X, T291X, I332X, G337X, I338X, and P339X of SEQ ID NO: 1 or a substitution at a position corresponding thereto.

3. The composition of claim 1 or 2, wherein the non-polar aliphatic amino acid is selected from proline (P), alanine (A), glycine (G), valine (V), leucine (L), and isoleucine (I).

4. The composition of claim 1 or 2, wherein X is selected from a non-polar amino acid, optionally selected from alanine (A), glycine (G), valine (V), leucine (L), isoleucine (I), proline (P), methionine (M), tryptophan (W), and phenylalanine (F), or a polar amino acid, optionally selected from serine(S), cysteine (C), asparagine (N), glutamine (Q), threonine (T), and tyrosine (Y), or a basic amino acid, optionally selected from arginine (R), histidine (H), or an acidic amino acid, optionally selected from aspartic acid (D), and glutamic acid (E), or a positively charged hydrophilic amino acid, optionally selected from lysine (K), arginine (R), and histidine (H).

5. The composition of any one of claims 1-4, wherein the helper enzyme comprises a substitution at S8X of SEQ ID NO: 1 or a substitution at a position corresponding thereto, optionally wherein X is a non-polar residue, optionally selected from alanine (A), glycine (G), valine (V), leucine (L), isoleucine (I), or a polar and neutral hydrophilic residue, optionally proline (P).

6. The composition of any one of claims 1-5, wherein the helper enzyme comprises a substitution at C13X of SEQ ID NO: 1 or a substitution at a position corresponding thereto, optionally wherein X is a positively charged hydrophilic amino acid, optionally selected from arginine (R), lysine (K), and histidine (H).

7. The composition of any one of claims 1-6, wherein the helper enzyme comprises a substitution at D416X of SEQ ID NO: 1 or a substitution at a position corresponding thereto, wherein X is a polar and positively charged hydrophilic residue, optionally selected from arginine (R) and lysine (K), or a polar and neutral of charge hydrophilic residue, optionally selected from asparagine (N), glutamine (Q), serine(S), threonine (T), proline (P), and cysteine (C).

8. The composition of any one of claims 1-7, wherein the helper enzyme comprises one, or two, or three, or four, or five, or six substitutions at positions selected from 333, 334, 335, 336, 2, 8, 13, 416, 282, 283, 284, 285, 286, 287, 288, 289, 290, 291, 332, 337, 338, and 339, or positions corresponding thereto relative to SEQ ID NO: 1.

9. The composition of any one of claims 1-8, wherein the helper enzyme comprises one, or two, or three, or four substitutions at positions selected from: 2, 8, 13, 416, and one or two substitutions at positions selected from 333, 334, 335, 336, 281, 282, 283, 284, 285, 286, 287, 288, 289, 290, 291, 332, 337, 338, and 339, or positions corresponding thereto relative to SEQ ID NO: 1.

10. The composition of any one of claims 1-9, wherein the helper enzyme comprises one or two, or three, or four substitutions selected from: S2A, S8P, C13R, D416A and D416N or substitutions corresponding thereto relative to SEQ ID NO: 1.

11. The composition of any one of claims 1-10, wherein the help enzyme comprises a substitution selected from S8P/C13R/D416N, S8P, C13R, D416N, S8P/C13R, S8P/D416N, and C13R/D416N or substitutions corresponding thereto relative to SEQ ID NO: 1.

12. The composition of any one of claims 1-11, wherein the helper enzyme comprises one or two substitutions selected from R333K, K334A, N335A, R336K, C282A, G283A, E284A, G285A, K286A, R287A, L288A, L289A, E290A, T291A, I332A, R333A, R333S, R336A, R336K, R336S, G337A, I338A, and P339A or substitutions corresponding thereto relative to SEQ ID NO: 1.

13. The composition of any one of claims 1-12, wherein the composition comprises one or two, or three, or four substitutions selected from: S2A, S8P, C13R, D416A and D416N or substitutions corresponding thereto relative to SEQ ID NO: 1, and one or two substitutions selected from R333K, K334A, N335A, R336K, C282A, G283A, E284A, G285A, K286A, R287A, L288A, L289A, E290A, T291A, I332A, R333A, R333S, R336A, R336S, G337A, I338A, and P339A or substitutions corresponding thereto relative to SEQ ID NO: 1.

14. The composition of any one of claims 1-13, wherein the composition one, or two, or three, or four, or five, or six substitutions selected from S2A, S8P, C13R, Y281A, C282A, G283A, E284A, G285A, K286A, R287A, L288A, L289A, E290A, T291A, I332A, R333A, R333K, R333S, K334A, N335A, R336A, R336K, R336S, G337A, I338A, P339A, D416A and D416N or substitutions corresponding thereto relative to SEQ ID NO: 1.

15. The composition of any one of claims 1-14, wherein the helper enzyme comprises a substitution selected from TABLE 1, TABLE 1A, TABLE 2 and/or TABLE 2A or a substitution at positions corresponding thereto relative to SEQ ID NO: 1.

16. The composition of any one of claims 1-15, wherein the helper enzyme comprises S8P/C13R/D416N/R333K/K334A/N335A/R336K, S8P/C13R/D416N/R333K/N335A/R336K, D416N/I338A, D416N/R333K, D416N/R333K/R336K, D416N/R336S, D416N/R333K/K334A, D416N/R333K/K334A/R336K, D416N/R333K/N335A, D416N/R333K/N335A/R336K, D416N/R333K/K334A/N335A, D416N/R333K/K334A/N335A/R336K, S8P/C13R/D416N/Y281A, S8P/C13R/D416N/K334A, S8P/C13R/D416N/K334A/N335A, S8P/C13R/D416N/K334A/R336A, S8P/C13R/D416N/N335A, S8P/C13R/D416N/N335N/R336A, S8P/C13R/D416N/R333A, S8P/C13R/D416N/R333A/K334A, S8P/C13R/D416N/R333A/N335A, S8P/C13R/D416N/R333A/R336A, S8P/C13R/D416N/R336A, S8P/C13R/D416N/Y281A/K334A, S8P/C13R/D416N/Y281A/K334A/N335A, S8P/C13R/D416N/K334A/N335A/R336K, S8P/C13R/D416N/K334A/N335A/R336S, S8P/C13R/D416N/K334A/R336K, S8P/C13R/D416N/K334A/R336S, S8P/C13R/D416N/N335A/R336K, S8P/C13R/D416N/N335A/R336S, S8P/C13R/D416N/R333K, S8P/C13R/D416N/R333K/K334A, S8P/C13R/D416N/R333K/K334A/N335A, S8P/C13R/D416N/R333K/K334A/N335A/R336S, S8P/C13R/D416N/R333K/K334A/R336A, S8P/C13R/D416N/R333K/K334A/R336K, S8P/C13R/D416N/R333K/K334A/R336S, S8P/C13R/D416N/R333K/N335A, S8P/C13R/D416N/R333K/N335A/R336S, S8P/C13R/D416N/R333K/R336K, S8P/C13R/D416N/R333K/R336S, S8P/C13R/D416N/R333S, S8P/C13R/D416N/R333S/K334A, S8P/C13R/D416N/R333S/K334A/N335A, S8P/C13R/D416N/R333S/K334A/N335A/R336K, S8P/C13R/D416N/R333S/K334A/N335A/R336S, S8P/C13R/D416N/R333S/K334A/R336K, S8P/C13R/D416N/R333S/K334A/R336S, S8P/C13R/D416N/R333S/N335A, S8P/C13R/D416N/R333S/N335A/R336K, S8P/C13R/D416N/R333S/N335A/R336S, S8P/C13R/D416N/R333S/R336K, S8P/C13R/D416N/R333S/R336S, S8P/C13R/D416N/R336K, S8P/C13R/D416N/R336S, or substitutions corresponding thereto relative to SEQ ID NO: 1.

17. The composition of any one of claims 1-16, wherein the helper enzyme comprises E2C/S8P/C13R/D416N/R333K/K334A/N335A/R336K, E2C/S8P/C13R/D416N/R333K/N335A/R336K, E2C/S8P/C13R/D416N/K334A, E2C/S8P/C13R/D416N/K334A/N335A, E2C/S8P/C13R/D416N/K334A/R336A, E2C/S8P/C13R/D416N/N335A, E2C/S8P/C13R/D416N/N335A/R336A, E2C/S8P/C13R/D416N/R333A, E2C/S8P/C13R/D416N/R333A/K334A, E2C/S8P/C13R/D416N/R333A/N335A, E2C/S8P/C13R/D416N/R333A/K336A, E2C/S8P/C13R/D416N/R336A, E2C/S8P/C13R/D416N/R281A, E2C/S8P/C13R/D416N/Y281A/K334A, E2C/S8P/C13R/D416N/Y281A/K334A/N335A, E2C/S8P/C13R/D416N/Y281A/N335A, E2C/S8P/C13R/D416N/K334A/N335A/R336K, E2C/S8P/C13R/D416N/K334A/N335A/R336S, E2C/S8P/C13R/D416N/K334A/R336K, E2C/S8P/C13R/D416N/K334A/R336S, E2C/S8P/C13R/D416N/N335A/R336K, E2C/S8P/C13R/D416N/N335A/R336S, E2C/S8P/C13R/D416N/R333K, E2C/S8P/C13R/D416N/R333K/K334A, E2C/S8P/C13R/D416N/R333K/K334A/N335A, E2C/S8P/C13R/D416N/R333K/K334A/N335A/R336S, E2C/S8P/C13R/D416N/R333K/K334A/R336A, E2C/S8P/C13R/D416N/R333K/K334A/R336K, E2C/S8P/C13R/D416N/R333K/K334A/R336S, E2C/S8P/C13R/D416N/R333K/N335A, E2C/S8P/C13R/D416N/R333K/N335A/R336S, E2C/S8P/C13R/D416N/R333K/R336K, E2C/S8P/C13R/D416N/R333K/R336S, E2C/S8P/C13R/D416N/R333S, E2C/S8P/C13R/D416N/R333S/K334A, E2C/S8P/C13R/D416N/R333S/K334A/N335A, E2C/S8P/C13R/D416N/R333S/K334A/N335A/R336K, E2C/S8P/C13R/D416N/R333S/K334A/N335A/R336S, E2C/S8P/C13R/D416N/R333S/K334A/R336K, E2C/S8P/C13R/D416N/R333S/K334A/R336S, E2C/S8P/C13R/D416N/R333S/N335A, E2C/S8P/C13R/D416N/R333S/N335A/R336K, E2C/S8P/C13R/D416N/R333S/N335A/R336S, E2C/S8P/C13R/D416N/R333S/R336K, E2C/S8P/C13R/D416N/R333S/R336S, E2C/S8P/C13R/D416N/R336K, E2C/S8P/C13R/D416N/R336S, or substitution corresponding thereto relative to SEQ ID NO: 1.

18. The composition of any one of claims 1-17, wherein the helper enzyme comprises a deletion of about 30, or about 40, or about 50, or about 60, or about 70, or about 80, or about 90, or about 100 amino acids from an N-terminus or a C-terminus.

19. The composition of any one of claims 1-18, wherein the helper enzyme comprises a deletion at positions about 1-35, or about 1-45, or about 1-55, or about 1-65, or about 1-75, or about 1-85, or about 1-95, or about 1-105, or about 2-35, or about 2-45, or about 2-68 or positions corresponding thereto, wherein the positions are relative to SEQ ID NO: 1.

20. The composition of claim 18 or 19, wherein the N terminal deletion yields reduced or ablated off-target effects of the enzyme compared to the enzyme without the N terminal deletion.

21. The composition of any one of claims 1-20, wherein the helper enzyme comprising the N terminal deletion has a sequence selected from SEQ ID NO: 504, 506, or 508, or a sequence having at least about 95%, or at least about 96%, at least about 97%, at least about 98%, at least about 99% identity thereto.

22. The composition of any one of claims 18-21, wherein the helper enzyme comprising an N terminal deletion is fused to a DNA binding domain, optionally wherein the DNA binding domain comprises transcription activator-like effectors (TALEs), and/or zinc finger (ZnF).

23. The composition of any one of claims 1-22, wherein the helper enzyme further comprises a targeting element.

24. The composition of any one of claims 1-23, wherein the helper enzyme is suitable of inserting a donor nucleic acid comprising a transgene in a genomic safe harbor site (GSHS).

25. The composition of claim 24, wherein the GSHS is in an open chromatin location in a chromosome.

26. The composition of claim 24 or 25, wherein the GSHS comprises a heterodimeric site located in chromosome 6.

27. The composition of any one of claims 24-26, wherein the GSHS is selected from adeno-associated virus site 1 (AAVS1), chemokine (C—C motif) receptor 5 (CCR5) gene, HIV-1 coreceptor, and human Rosa26 locus.

28. The composition of any one of claims 23-27, wherein the GSHS comprises one or more TTAA integration sites, optionally wherein the TTAA integration sites comprise ttTTAAaa (SEQ ID NO: 880) or taTTAAta (SEQ ID NO: 881) sites.

29. The composition of claim 28, wherein the targeting element directs the helper enzyme to either one or more nucleic acid binding sites that are upstream and/or downstream of the TTAA integration sites or to the TTAA integration sites.

30. The composition of claim 28, wherein the targeting element directs the helper enzyme to one or more nucleic acid binding sites that are upstream and/or downstream of the TTAA integration sites and within about 5 to about 30 base pairs of the TTAA integration sites or within about 15 to about 19 base pairs of the TTAA integration sites.

31. The composition of claim 28, wherein the targeting element directs the helper enzyme to two nucleic acid binding sites of the TTAA integration sites, wherein a first site is upstream of TTAA and within about 5 to about 30 base pairs or about 15 to about 19 base pairs of the TTAA and a second site is downstream of TTAA and within about 5 to about 30 base pairs or about 15 to about 19 base pairs of the TTAA.

32. The composition of claim 28, wherein the targeting element directs the helper enzyme to identical targeting regions both upstream and downstream of the TTAA integration sites.

33. The composition of any one of claims 23-32, wherein the targeting element is or comprises one or more of a transcription activator-like effector (TALE) DNA binding domain (DBD), a Cas enzyme, which is optionally catalytically inactive and which is optionally associated with a guide RNA (gRNA), a Zinc finger (ZnF), a catalytically inactive transcription factor, catalytically inactive nickase, a transcriptional activator, a transcriptional repressor, a recombinase, a DNA methyltransferase, a histone methyltransferase, a paternally expressed gene 10 (PEG10), and a transposon-encoded polypeptide D (TnsD) or a variant thereof.

34. The composition of any one of claims 23-33, wherein the targeting element is inserted after residue S60, or after residue E70, or after residue L80, or after residue N90, or after residue D100, relative to SEQ ID NO: 1, optionally by way of a linker.

35. The composition of claim 34, wherein the targeting element is inserted immediately after residue G66 relative to SEQ ID NO: 1, optionally by way of a linker.

36. The composition of claim 23, wherein the targeting element is or comprises a TALE DBD.

37. The composition of claim 36, wherein the TALE DBD comprises one or more repeat sequences.

38. The composition of claim 37, wherein the TALE DBD comprises about 14, or about 15, or about, 16, or about 17, or about 18, or about 18.5 repeat sequences.

39. The composition of claim 37 or claim 38, wherein the repeat sequences each independently comprises about 33 or 34 amino acids.

40. The composition of claim 39, wherein the repeat sequences each independently comprises a repeat variable di-residue (RVD) at residue 12 or 13 of the 33 or 34 amino acids, respectively.

41. The composition of claim 40, wherein the RVD recognizes one base pair in a target nucleic acid sequence.

42. The composition of claim 40 or claim 41, wherein the RVD recognizes a C residue in the target nucleic acid sequence and is selected from HD, N (gap), HA, ND, and HI.

43. The composition of claim 40 or claim 41, wherein the RVD recognizes a G residue in the target nucleic acid sequence and is selected from NN, NH, NK, HN, and NA.

44. The composition of claim 40 or claim 41, wherein the RVD recognizes an A residue in the target nucleic acid sequence and is selected from NI and NS.

45. The composition of claim 40 or claim 41, wherein the RVD recognizes a T residue in the target nucleic acid sequence and is selected from NG, HG, H (gap), and IG.

46. The composition of claim 23, wherein the targeting element is or comprises a Cas9 enzyme associated with a gRNA or a CasX enzyme associated with a gRNA.

47. The composition of claim 46, wherein the Cas9 enzyme associated with a gRNA comprises a catalytically inactive dCas9 associated with a gRNA or an inactive dCasX associated with a gRNA.

48. The composition of claim 47, wherein catalytically inactive dCas9 comprises at least about 90%, or at least about 95%, or at least about 96%, or at least about 97%, or at least about 98%, or at least about 99% identity to an amino acid sequence of SEQ ID NO: 6 or a nucleic acid comprising a nucleotide sequence of SEQ ID NO: 5 or a codon-optimized form thereof.

49. The composition of claim 46, wherein the target element is or comprises a CasX enzyme associated with a gRNA, optionally wherein the catalytic inactive dCasX.

50. The composition of claim 23, wherein the targeting element is or comprises a Cas12 enzyme associated with a gRNA.

51. The composition of claim 50, wherein the targeting element comprises a catalytically inactive Cas12 associated with a gRNA, optionally wherein the catalytically inactive Cas12 is dCas12j or dCas12a.

52. The composition of claim 23, the targeting element is or comprises a TnsC, TnsB, TnsA, TniQ, Cas6, Cas7, Cas8 enzyme associated with a gRNA.

53. The composition of claim 52, wherein the targeting element comprises a TniQ subdomain of TnsD.

54. The composition of any one of claims 1-53, wherein the composition comprises a linker connecting the helper enzyme and the targeting element.

55. The composition of claim 54, wherein the linker is a covalent linker or a non-covalent linker.

56. The composition of claim 54, wherein the linker comprises less than about 25 amino acids or 75 nucleotides.

57. The composition of claim 54, wherein the linker comprises about 10 amino acids to about 20 amino acids or about 12 amino acids to about 15 amino acids, or about 30 nucleotides to about 60 nucleotides or about 36 nucleotides to about 45 nucleotides.

58. The composition of any one of claims 54-57, wherein the linker is substantially comprised of glycine (G) and serine(S) residues.

59. The composition of any one of claims 54-58, wherein the linker is or comprises (GSS) 4 or the linker is GS flanked on either side of a DNA binding domain, optionally TALE and ZnF.

60. The composition of claim 59, wherein the linker comprises an amino acid sequence of AKLAGGAPAVGGGPKAADKFAATGGS (SEQ ID NO: 8) or a variant thereof having a substitution or deletion.

61. The composition of any one of claims 54-60, wherein the linker connects the targeting element to the N-terminus of the helper enzyme or connects the targeting element within the helper enzyme.

62. The composition of any one of claims 54-61, wherein the linker is a non-covalent linker.

63. The composition of claim 62, wherein the non-covalent linker comprises an epitope tag, optionally wherein the epitope tag comprises an ALFA tag.

64. The composition of claim 63, wherein the ALFA tag further comprises recombinant variable domains of heavy-chain-only antibodies (VHH) (NANOBODY).

65. The composition of any one of claims 54-64, wherein the linker is inserted after residue S60, or after residue E70, or after residue L80, or after residue N90, or after residue D100 relative to SEQ ID NO: 1.

66. The composition of claim 65, wherein the linker is inserted immediately after residue G66 relative to SEQ ID NO: 1.

67. The composition of any one of claims 1-66, wherein the composition is a nucleic acid, optionally an RNA.

68. The composition of claim 1-67, wherein the RNA is or comprises messenger RNA (mRNA).

69. The composition of claim 1-68, wherein the mRNA is or comprises modified mRNA (mmRNA).

70. The composition of claim 1-69, wherein the mmRNA comprises one or more of a 5′-m7G cap (cap0, cap1, or cap2), a pseudouridine or n-methyl-pseudouridine substitution, and a poly-A tail of about 30, or of about 50, or of about 100, or of about 150 nucleotides in length.

71. The composition of any one of claims 1-70, wherein the composition further comprises a donor nucleic acid or is suitable for insertion of a donor nucleic acid, optionally wherein the donor nucleic acid is a transposon.

72. The composition of any one of claims 1-71, wherein the helper enzyme is suitable of inserting a donor nucleic acid comprising a transgene in a genomic safe harbor site (GSHS) and/or wherein the targeting element is suitable for directing the helper enzyme to a GSHS.

73. The composition of any one of claims 1-72, wherein a donor DNA and a helper RNA are suitable for transfection at a donor DNA to helper RNA ratio of about 1 to about 4, or about 1 to about 2, or about 1 to about 1.

74. The composition of any one of claims 1-73, further comprising a nucleic acid encoding a donor comprising a transgene to be integrated, optionally wherein the transgene is defective or substantially absent in a disease state.

75. The composition of claim 74, wherein the transgene comprises a cargo nucleic acid sequence and a first and a second donor end sequences.

76. The composition of claim 75, wherein the cargo nucleic acid sequence is flanked by the first and the second donor end sequences.

77. The composition of any one of claims 1-76, wherein the enzyme or variant thereof is incorporated into a vector or a vector-like particle.

78. The composition of any one of claims 1-77, wherein the vector or a vector-like particle comprises one or more expression cassettes.

79. The composition of claim 78, wherein the vector or a vector-like particle comprises one expression cassette.

80. The composition of claim 78 or 79, wherein the expression cassette further comprises the enzyme or variant thereof, the transgene, the donor end sequences, or a combination thereof.

81. The composition of claim 78, wherein the enzyme or variant thereof, the transgene, the donor end sequences, or a combination thereof are incorporated into one or more vectors or vector-like particles.

82. The composition of claim 78, wherein the enzyme or variant thereof, the transgene, the donor end sequences, or combination thereof are incorporated into a same vector or vector-like particle.

83. The composition of claim 78, wherein the enzyme or variant thereof, the transgene, the donor end sequences, or combination thereof is incorporated into different vectors or vector-like particles.

84. The composition of any one of claims 77-83, wherein the vector or vector-like particle is nonviral.

85. The composition of any one of claims 71-84, wherein the donor is under the control of at least one tissue-specific promoter.

86. The composition of claim 85, wherein at least one tissue-specific promoter is a single promoter.

87. The composition of claim 85, wherein at least one tissue-specific promoter is under the control of a dual promoter or a tandem promoter.

88. The composition of any one of claims 72-87, wherein the transgene to be integrated comprises at least one gene of interest.

89. The composition of any one of claims 72-87, wherein the transgene to be integrated comprises one gene of interest.

90. The composition of any one of claims 72-87, wherein the transgene to be integrated comprises two or more genes of interest.

91. A host cell comprising the composition any one of claims 1-90.

92. The composition of any one of claims 1-90, wherein the composition is encapsulated in a lipid nanoparticle (LNP).

93. The composition of any one of claims 1-90, wherein the polynucleotide encoding the enzyme or variant thereof and the polynucleotide encoding the donor are in the form of the same LNP, optionally in a co-formulation.

94. The composition of claim 92 or claim 93, wherein the LNP comprises one or more lipids selected from 1,2-dioleoyl-3-trimethylammonium propane (DOTAP), a cationic cholesterol derivative mixed with dimethylaminoethane-carbamoyl (DC-Chol), phosphatidylcholine (PC), triolein (glyceryl trioleate), and 1,2-distearoyl-sn-glycero-3-phosphoethanolamine-N-[carboxy (polyethylene glycol)-2000] (DSPE-PEG), 1,2-dimyristoyl-rac-glycero-3-methoxypolyethyleneglycol-2000 (DMG-PEG 2K), and 1,2 distearol-sn-glycerol-3phosphocholine (DSPC) and/or comprising of one or more molecules selected from polyethylenimine (PEI) and poly(lactic-co-glycolic acid) (PLGA), and N-Acetylgalactosamine (GalNAc).

95. A method for inserting a gene into the genome of a cell, comprising contacting a cell with the composition of any one of claims 1-90, 92-94 or host cell of claim 90.

96. A method for treating a disease or disorder ex vivo, comprising contacting a cell with the composition of any one of claims 1-90, 92-94 or host cell of claim 90 and administering the cell to a subject in need thereof.

97. A method for treating a disease or disorder in vivo, comprising administering the composition of any one of claims 1-90, 92-94 or host cell of claim 90 to a subject in need thereof.

98. A composition comprising a helper enzyme or a nucleic acid encoding the helper enzyme comprising:
an amino acid sequence having at least about 80% sequence identity to SEQ ID NO: 1 and a non-polar aliphatic amino acid at position 2 of SEQ ID NO: 1, or a position corresponding thereto, and amino acid substitutions comprising S8X, C13X, R333K, K334A, N335A, R336K, and D416X of SEQ ID NO: 1, or a substitution at a position corresponding thereto.

99. A composition comprising (a) a helper enzyme or a nucleic acid encoding the helper enzyme and (b) a targeting element or a nucleic acid encoding the targeting element, wherein:
the helper enzyme comprises an amino acid sequence having at least about 80% sequence identity to SEQ ID NO: 1 and has a non-polar aliphatic amino acid at position 2 of SEQ ID NO: 1 and a substitution at a position corresponding thereto, and amino acid substitutions comprising S8X, C13X, R333K, K334A, N335A, R336K, and D416X of SEQ ID NO: 1, or a substitution at a position corresponding thereto.

100. A composition comprising a helper enzyme or a nucleic acid encoding the helper enzyme comprising:
an amino acid sequence having at least about 80% sequence identity to SEQ ID NO: 1 and a non-polar aliphatic amino acid at position 2 of SEQ ID NO: 1, or a position corresponding thereto, and amino acid substitutions comprising S8X, C13X, R333K, N335A, R336K, and D416X of SEQ ID NO: 1, or a substitution at a position corresponding thereto.

101. A composition comprising (a) a helper enzyme or a nucleic acid encoding the helper enzyme and (b) a targeting element or a nucleic acid encoding the targeting element, wherein:
an amino acid sequence having at least about 80% sequence identity to SEQ ID NO: 1 and a non-polar aliphatic amino acid at position 2 of SEQ ID NO: 1, or a position corresponding thereto, and amino acid substitutions comprising S8X, C13X, R333K, N335A, R336K, and D416X of SEQ ID NO: 1, or a substitution at a position corresponding thereto.

102. A method of designing a transposase for binding identical target sites upstream and downstream of a target nucleic acid site, comprising:
a) obtaining the transposase, the transposase being modified to have reduced or ablated inherent nucleic acid binding ability and having at least about 90%, or at least about 93%, at least about 95%, at least about 97%, at least about 98%, at least about 99% identity to the amino acid sequence of SEQ ID NO: 1,
b) fusing or associating the transposase with at least two targeting elements, each targeting element being fused at or immediately after G66 relative to SEQ ID NO: 1, optionally wherein the fusing or associating the transposase with at least two targeting elements is by way of a linker,
wherein the target nucleic acid site is a genomic safe harbor site of or comprising TTAA.

103. The method of claim 102, wherein the linker is a covalent linker or a non-covalent linker.

104. The method of claim 102, wherein the linker comprises less than about 25 amino acids or 75 nucleotides.

105. The method of claim 102, wherein the linker comprises about 10 amino acids to about 20 amino acids or about 12 amino acids to about 15 amino acids, or about 30 nucleotides to about 60 nucleotides or about 36 nucleotides to about 45 nucleotides.

106. The method of any one of claims 102-105, wherein the linker is substantially comprised of glycine (G) and serine(S) residues.

107. The method of any one of claims 102-106, wherein the linker is or comprises (GSS) 4 or the linker is GS flanked on either side of a targeting element, optionally TALE and ZnF.

108. The method of claim 102, wherein the linker comprises an amino acid sequence of AKLAGGAPAVGGGPKAADKFAATGGS (SEQ ID NO: 8) or a variant thereof having a substitution or deletion.

109. The method of any one of claims 102-108, wherein the linker connects the targeting element to the N-terminus of the helper enzyme or connects the targeting element within the helper enzyme.

110. The method of any one of claims 102-109, wherein the linker is a non-covalent linker.

111. The method of claim 110, wherein the non-covalent linker comprises an epitope tag, optionally wherein the epitope tag comprises an ALFA tag.

112. The method of claim 111, wherein the ALFA tag further comprises recombinant variable domains of heavy-chain-only antibodies (VHH) (NANOBODY).

113. The method of any one of claims 102-112, wherein the targeting elements are oriented on opposite sides of the transposase.


SEQUENCE LISTING (1178 sequences)

SEQ ID NO: 1 - protein, 572 aa, synthetic construct
  REGION 1..572: note = synthetic polymer
MAQHSDYSDDEFCADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNY

SEQ ID NO: 2 - protein, 572 aa, synthetic construct
MAQHSDYPDDEFRADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNY

SEQ ID NO: 3 - DNA, 157 bp, synthetic construct
  misc_feature 1..157: note = synthetic polymer
  source 1..157: mol_type = other DNA
ttaacacttggattgcgggaaacgagttaagtcggctcgcgtgaattgcgcgtactccgcgggagccgtcttaactcggttcatatagatttgcggtggagtgcgggaaacgtgtaaactcgggccgattgtaactgcgtattaccaaatatttgtt

SEQ ID NO: 4 - DNA, 212 bp, synthetic construct
  misc_feature 1..212: note = synthetic polymer
  source 1..212: mol_type = other DNA
aattatttatgtactgaatagataaaaaaatgtctgtgattgaataaattttcattttttacacaagaaaccgaaaatttcatttcaatcgaacccatacttcaaaagatataggcattttaaactaactctgattttgcgcgggaaacctaaataattgcccgcgccatcttatattttggcgggaaattcacccgacaccgtagtgttaa

SEQ ID NO: 5 - DNA, 4104 bp, synthetic construct
  misc_feature 1..4104: note = synthetic polymer
  source 1..4104: mol_type = other DNA
atggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctggaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 6 - protein, 1368 aa, synthetic construct
  REGION 1..1368: note = synthetic polymer
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVAAIVPQSFLKDDSIDNKVLTRSDKARGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 7 - DNA, 1716 bp, synthetic construct
  misc_feature 1..1716: note = synthetic polymer
  source 1..1716: mol_type = other DNA
atggcccagcacagcgactaccccgacgacgagttcagagccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattac

SEQ ID NO: 8 - protein, 26 aa, synthetic construct
AKLAGGAPAVGGGPKAADKFAATGGS

SEQ ID NO: 9 - skipped

SEQ ID NO: 10 - DNA, 1719 bp, synthetic construct
  source 1..1719: mol_type = other DNA
atggcccagcacagcgactaccccgacgacgagttcagagccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattactag

SEQ ID NO: 11 - skipped

SEQ ID NO: 12 - protein, 508 aa, synthetic construct
  REGION 1..508: note = synthetic polymer
MRNFPVPYSNELIYSTIARAGVYQGIVSPKQLLDEVYGNRKVVATLGLPSHLGVIARHLHQTGRYAVQQLIYEHTLFPLYAPFVGKERRDEAIRLMEYQAQGAVHLMLGVAASRVKSDNRFRYCPDCVALQLNRYGEAFWQRDWYLPALPYCPKHGALVFFDRAVDDHRHQFWALGHTELLSDYPKDSLSQLTALAAYIAPLLDAPRAQELSPSLEQWTLFYQRLAQDLGLTKSKHIRHDLVAERVRQTFSDEALEKLDLKLAENKDTCWLKSIFRKHRKAFSYLQHSIVWQALLPKLTVIEALQQASALTEHSITTRPVSQSVQPNSEDLSVKHKDWQQLVHKYQGIKAARQSLEGGVLYAWLYRHDRDWLVHWNQQHQQERLAPAPRVDWNQRDRIAVRQLLRIIKRLDSSLDHPRATSSWLLKQTPNGTSLAKNLQKLPLVALCLKRYSESVEDYQIRRISQAFIKLKQEDVELRRWRLLRSATLSKERITEEAQRFLEMVYGEE

SEQ ID NO: 13-22 - skipped (10 entries)

SEQ ID NO: 23 - DNA, 19 bp, synthetic construct
  misc_feature 1..19: note = synthetic polymer
  source 1..19: mol_type = other DNA
tggccggcctgaccactgg

SEQ ID NO: 24 - DNA, 19 bp, synthetic construct
  misc_feature 1..19: note = synthetic polymer
  source 1..19: mol_type = other DNA
tgaaggcctggccggcctg

SEQ ID NO: 25 - DNA, 19 bp, synthetic construct
  misc_feature 1..19: note = synthetic polymer
  source 1..19: mol_type = other DNA
tgagcactgaaggcctggc

SEQ ID NO: 26 - DNA, 19 bp, synthetic construct
  misc_feature 1..19: note = synthetic polymer
  source 1..19: mol_type = other DNA
tccactgagcactgaaggc

SEQ ID NO: 27 - DNA, 19 bp, synthetic construct
  misc_feature 1..19: note = synthetic polymer
  source 1..19: mol_type = other DNA
tggtttccactgagcactg

SEQ ID NO: 28 - DNA, 19 bp, synthetic construct
  misc_feature 1..19: note = synthetic polymer
  source 1..19: mol_type = other DNA
tggggaaaatgacccaaca

SEQ ID NO: 29 - DNA, 19 bp, synthetic construct
  misc_feature 1..19: note = synthetic polymer
  source 1..19: mol_type = other DNA
taggacagtggggaaaatg

SEQ ID NO: 30 - DNA, 19 bp, synthetic construct
  misc_feature 1..19: note = synthetic polymer
  source 1..19: mol_type = other DNA
tccagggacacggtgctag

SEQ ID NO: 31 - DNA, 19 bp, synthetic construct
  misc_feature 1..19: note = synthetic polymer
  source 1..19: mol_type = other DNA
tcagagccaggagtcctgg

SEQ ID NO: 32 - DNA, 19 bp, synthetic construct
  misc_feature 1..19: note = synthetic polymer
  source 1..19: mol_type = other DNA
tccttcagagccaggagtc

SEQ ID NO: 33 - DNA, 19 bp, synthetic construct
  misc_feature 1..19: note = synthetic polymer
  source 1..19: mol_type = other DNA
tcctccttcagagccagga

SEQ ID NO: 34 - DNA, 19 bp, synthetic construct
  misc_feature 1..19: note = synthetic polymer
  source 1..19: mol_type = other DNA
tccagcccctcctccttca

SEQ ID NO: 35 - DNA, 19 bp, synthetic construct
  misc_feature 1..19: note = synthetic polymer
  source 1..19: mol_type = other DNA
tccgagcttgacccttgga

SEQ ID NO: 36 - DNA, 19 bp, synthetic construct
  misc_feature 1..19: note = synthetic polymer
  source 1..19: mol_type = other DNA
tggtttccgagcttgaccc

SEQ ID NO: 37 - DNA, 19 bp, synthetic construct
  misc_feature 1..19: note = synthetic polymer
  source 1..19: mol_type = other DNA
tggggtggtttccgagctt

SEQ ID NO: 38 - DNA, 19 bp, synthetic construct
  misc_feature 1..19: note = synthetic polymer
  source 1..19: mol_type = other DNA
tctgctggggtggtttccg

SEQ ID NO: 39 - DNA, 19 bp, synthetic construct
  misc_feature 1..19: note = synthetic polymer
  source 1..19: mol_type = other DNA
tgcagagtatctgctgggg

SEQ ID NO: 40 - DNA, 14 bp, synthetic construct
  misc_feature 1..14: note = synthetic polymer
  source 1..14: mol_type = other DNA
ccaatcccctcagt

SEQ ID NO: 41 - DNA, 15 bp, synthetic construct
  misc_feature 1..15: note = synthetic polymer
  source 1..15: mol_type = other DNA
cagtgctcagtggaa

SEQ ID NO: 42 - DNA, 18 bp, synthetic construct
  misc_feature 1..18: note = synthetic polymer
  source 1..18: mol_type = other DNA
gaaacatccggcgactca

SEQ ID NO: 43 - DNA, 19 bp, synthetic construct
  misc_feature 1..19: note = synthetic polymer
  source 1..19: mol_type = other DNA
tcgcccctcaaatcttaca

SEQ ID NO: 44 - DNA, 19 bp, synthetic construct
  misc_feature 1..19: note = synthetic polymer
  source 1..19: mol_type = other DNA
tcaaatcttacagctgctc

SEQ ID NO: 45 - DNA, 19 bp, synthetic construct
  misc_feature 1..19: note = synthetic polymer
  source 1..19: mol_type = other DNA
tcttacagctgctcactcc

SEQ ID NO: 46 - DNA, 19 bp, synthetic construct
  misc_feature 1..19: note = synthetic polymer
  source 1..19: mol_type = other DNA
tacagctgctcactcccct

SEQ ID NO: 47 - DNA, 19 bp, synthetic construct
  misc_feature 1..19: note = synthetic polymer
  source 1..19: mol_type = other DNA
tgctcactcccctgcaggg

SEQ ID NO: 48 - DNA, 19 bp, synthetic construct
  misc_feature 1..19: note = synthetic polymer
  source 1..19: mol_type = other DNA
tcccctgcagggcaacgcc

SEQ ID NO: 49 - DNA, 19 bp, synthetic construct
  misc_feature 1..19: note = synthetic polymer
  source 1..19: mol_type = other DNA
tgcagggcaacgcccaggg

SEQ ID NO: 50 - DNA, 19 bp, synthetic construct
  misc_feature 1..19: note = synthetic polymer
  source 1..19: mol_type = other DNA
tctcgattatgggcgggat

SEQ ID NO: 51 - DNA, 19 bp, synthetic construct
  misc_feature 1..19: note = synthetic polymer
  source 1..19: mol_type = other DNA
tcgcttctcgattatgggc

SEQ ID NO: 52 - DNA, 19 bp, synthetic construct
  misc_feature 1..19: note = synthetic polymer
  source 1..19: mol_type = other DNA
tgtcgagtcgcttctcgat

SEQ ID NO: 53 - DNA, 19 bp, synthetic construct
  misc_feature 1..19: note = synthetic polymer
  source 1..19: mol_type = other DNA
tccatgtcgagtcgcttct

SEQ ID NO: 54 - DNA, 19 bp, synthetic construct
  misc_feature 1..19: note = synthetic polymer
  source 1..19: mol_type = other DNA
tcgcctccatgtcgagtcg

SEQ ID NO: 55 - DNA, 19 bp, synthetic construct
  misc_feature 1..19: note = synthetic polymer
  source 1..19: mol_type = other DNA
tcgtcatcgcctccatgtc

SEQ ID NO: 56 - DNA, 19 bp, synthetic construct
  misc_feature 1..19: note = synthetic polymer
  source 1..19: mol_type = other DNA
tgatctcgtcatcgcctcc

SEQ ID NO: 57 - DNA, 14 bp, synthetic construct
  misc_feature 1..14: note = synthetic polymer
  source 1..14: mol_type = other DNA
gcttcagcttccta

SEQ ID NO: 58 - DNA, 14 bp, synthetic construct
  misc_feature 1..14: note = synthetic polymer
  source 1..14: mol_type = other DNA
ctgtgatcatgcca

SEQ ID NO: 59 - DNA, 16 bp, synthetic construct
  misc_feature 1..16: note = synthetic polymer
  source 1..16: mol_type = other DNA
acagtggtacacacct

SEQ ID NO: 60 - DNA, 15 bp, synthetic construct
  misc_feature 1..15: note = synthetic polymer
  source 1..15: mol_type = other DNA
ccaccccccactaag

SEQ ID NO: 61 - DNA, 14 bp, synthetic construct
  misc_feature 1..14: note = synthetic polymer
  source 1..14: mol_type = other DNA
cattggccgggcac

SEQ ID NO: 62 - DNA, 16 bp, synthetic construct
  misc_feature 1..16: note = synthetic polymer
  source 1..16: mol_type = other DNA
gcttgaacccaggaga

SEQ ID NO: 63 - DNA, 17 bp, synthetic construct
  misc_feature 1..17: note = synthetic polymer
  source 1..17: mol_type = other DNA
acacccgatccactggg

SEQ ID NO: 64 - DNA, 14 bp, synthetic construct
  misc_feature 1..14: note = synthetic polymer
  source 1..14: mol_type = other DNA
gctgcatcaacccc

SEQ ID NO: 65 - DNA, 16 bp, synthetic construct
  misc_feature 1..16: note = synthetic polymer
  source 1..16: mol_type = other DNA
gccacaaacagaaata

SEQ ID NO: 66 - DNA, 15 bp, synthetic construct
  misc_feature 1..15: note = synthetic polymer
  source 1..15: mol_type = other DNA
ggtggctcatgcctg

SEQ ID NO: 67 - DNA, 16 bp, synthetic construct
  misc_feature 1..16: note = synthetic polymer
  source 1..16: mol_type = other DNA
gatttgcacagctcat

SEQ ID NO: 68 - DNA, 15 bp, synthetic construct
  misc_feature 1..15: note = synthetic polymer
  source 1..15: mol_type = other DNA
aagctctgaggagca

SEQ ID NO: 69 - DNA, 13 bp, synthetic construct
  misc_feature 1..13: note = synthetic polymer
  source 1..13: mol_type = other DNA
ccctagctgtccc

SEQ ID NO: 70 - DNA, 14 bp, synthetic construct
  misc_feature 1..14: note = synthetic polymer
  source 1..14: mol_type = other DNA
gcctagcatgctag

SEQ ID NO: 71 - DNA, 15 bp, synthetic construct
  misc_feature 1..15: note = synthetic polymer
  source 1..15: mol_type = other DNA
atgggcttcacggat

SEQ ID NO: 72 - DNA, 14 bp, synthetic construct
  misc_feature 1..14: note = synthetic polymer
  source 1..14: mol_type = other DNA
gaaactatgcctgc

SEQ ID NO: 73 - DNA, 14 bp, synthetic construct
  misc_feature 1..14: note = synthetic polymer
  source 1..14: mol_type = other DNA
gcaccattgctccc

SEQ ID NO: 74 - DNA, 14 bp, synthetic construct
  misc_feature 1..14: note = synthetic polymer
  source 1..14: mol_type = other DNA
gacatgcaactcag

SEQ ID NO: 75 - DNA, 14 bp, synthetic construct
  misc_feature 1..14: note = synthetic polymer
  source 1..14: mol_type = other DNA
acaccactaggggt

SEQ ID NO: 76 - DNA, 14 bp, synthetic construct
  misc_feature 1..14: note = synthetic polymer
  source 1..14: mol_type = other DNA
gtctgctagacagg

SEQ ID NO: 77 - DNA, 15 bp, synthetic construct
  misc_feature 1..15: note = synthetic polymer
  source 1..15: mol_type = other DNA
ggcctagacaggctg

SEQ ID NO: 78 - DNA, 15 bp, synthetic construct
  misc_feature 1..15: note = synthetic polymer
  source 1..15: mol_type = other DNA
gaggcattcttatcg

SEQ ID NO: 79 - DNA, 15 bp, synthetic construct
  misc_feature 1..15: note = synthetic polymer
  source 1..15: mol_type = other DNA
gcctggaaacgttcc

SEQ ID NO: 80 - DNA, 14 bp, synthetic construct
  misc_feature 1..14: note = synthetic polymer
  source 1..14: mol_type = other DNA
gtgctctgacaata

SEQ ID NO: 81 - DNA, 14 bp, synthetic construct
  misc_feature 1..14: note = synthetic polymer
  source 1..14: mol_type = other DNA
gttttgcagcctcc

SEQ ID NO: 82 - DNA, 15 bp, synthetic construct
  misc_feature 1..15: note = synthetic polymer
  source 1..15: mol_type = other DNA
acagctgtggaacgt

SEQ ID NO: 83 - DNA, 15 bp, synthetic construct
  misc_feature 1..15: note = synthetic polymer
  source 1..15: mol_type = other DNA
ggctctcttcctcct

SEQ ID NO: 84 - DNA, 15 bp, synthetic construct
  misc_feature 1..15: note = synthetic polymer
  source 1..15: mol_type = other DNA
ctatcccaaaactct

SEQ ID NO: 85 - DNA, 14 bp, synthetic construct
  misc_feature 1..14: note = synthetic polymer
  source 1..14: mol_type = other DNA
gaaaaactatgtat

SEQ ID NO: 86 - DNA, 15 bp, synthetic construct
  misc_feature 1..15: note = synthetic polymer
  source 1..15: mol_type = other DNA
aggcaggctggttga

SEQ ID NO: 87 - DNA, 14 bp, synthetic construct
  misc_feature 1..14: note = synthetic polymer
  source 1..14: mol_type = other DNA
caatacaaccacgc

SEQ ID NO: 88 - DNA, 15 bp, synthetic construct
  misc_feature 1..15: note = synthetic polymer
  source 1..15: mol_type = other DNA
atgacggactcaact

SEQ ID NO: 89 - DNA, 14 bp, synthetic construct
  misc_feature 1..14: note = synthetic polymer
  source 1..14: mol_type = other DNA
cacaacatttgtaa

SEQ ID NO: 90 - DNA, 14 bp, synthetic construct
  misc_feature 1..14: note = synthetic polymer
  source 1..14: mol_type = other DNA
atttccagtgcaca

SEQ ID NO: 91 - DNA, 20 bp, synthetic construct
  misc_feature 1..20: note = synthetic polymer
  source 1..20: mol_type = other DNA
gtttagctcacccgtgagcc

SEQ ID NO: 92 - DNA, 20 bp, synthetic construct
  misc_feature 1..20: note = synthetic polymer
  source 1..20: mol_type = other DNA
cccaatattattgttctctg

SEQ ID NO: 93 - DNA, 20 bp, synthetic construct
  misc_feature 1..20: note = synthetic polymer
  source 1..20: mol_type = other DNA
ggggtgggataggggatacg

SEQ ID NO: 94 - DNA, 20 bp, synthetic construct
  misc_feature 1..20: note = synthetic polymer
  source 1..20: mol_type = other DNA
ggatccccctctacatttaa

SEQ ID NO: 95 - DNA, 20 bp, synthetic construct
  misc_feature 1..20: note = synthetic polymer
  source 1..20: mol_type = other DNA
gtgatcttgtacaaatcatt

SEQ ID NO: 96 - DNA, 20 bp, synthetic construct
  misc_feature 1..20: note = synthetic polymer
  source 1..20: mol_type = other DNA
ctacacagaatctgttagaa

SEQ ID NO: 97 - DNA, 20 bp, synthetic construct
  misc_feature 1..20: note = synthetic polymer
  source 1..20: mol_type = other DNA
taagctagagaatagatctc

SEQ ID NO: 98 - DNA, 20 bp, synthetic construct
  misc_feature 1..20: note = synthetic polymer
  source 1..20: mol_type = other DNA
tcaatacacttaatgattta

SEQ ID NO: 99 - DNA, 25 bp, synthetic construct
  misc_feature 1..25: note = synthetic polymer
  source 1..25: mol_type = other DNA
caccgggagccacgaaaacagatcc

SEQ ID NO: 100 - DNA, 25 bp, synthetic construct
  misc_feature 1..25: note = synthetic polymer
  source 1..25: mol_type = other DNA
caccgcgaaaacagatccagggaca

SEQ ID NO: 101 - DNA, 25 bp, synthetic construct
  misc_feature 1..25: note = synthetic polymer
  source 1..25: mol_type = other DNA
caccgagatccagggacacggtgct

SEQ ID NO: 102 - DNA, 25 bp, synthetic construct
  misc_feature 1..25: note = synthetic polymer
  source 1..25: mol_type = other DNA
caccggacacggtgctaggacagtg

SEQ ID NO: 103 - DNA, 25 bp, synthetic construct
  misc_feature 1..25: note = synthetic polymer
  source 1..25: mol_type = other DNA
caccggaaaatgacccaacagcctc

SEQ ID NO: 104 - DNA, 25 bp, synthetic construct
  misc_feature 1..25: note = synthetic polymer
  source 1..25: mol_type = other DNA
caccggcctggccggcctgaccact

SEQ ID NO: 105 - DNA, 25 bp, synthetic construct
  misc_feature 1..25: note = synthetic polymer
  source 1..25: mol_type = other DNA
caccgctgagcactgaaggcctggc

SEQ ID NO: 106 - DNA, 25 bp, synthetic construct
  misc_feature 1..25: note = synthetic polymer
  source 1..25: mol_type = other DNA
caccgtggtttccactgagcactga

SEQ ID NO: 107 - DNA, 25 bp, synthetic construct
  misc_feature 1..25: note = synthetic polymer
  source 1..25: mol_type = other DNA
caccggatagccaggagtcctttcg

SEQ ID NO: 108 - DNA, 25 bp, synthetic construct
  misc_feature 1..25: note = synthetic polymer
  source 1..25: mol_type = other DNA
caccggcgcttccagtgctcagact

SEQ ID NO: 109 - DNA, 25 bp, synthetic construct
  misc_feature 1..25: note = synthetic polymer
  source 1..25: mol_type = other DNA
caccgcagtgctcagactagggaag

SEQ ID NO: 110 - DNA, 25 bp, synthetic construct
  misc_feature 1..25: note = synthetic polymer
  source 1..25: mol_type = other DNA
caccggcccctcctccttcagagcc

SEQ ID NO: 111 - DNA, 25 bp, synthetic construct
  misc_feature 1..25: note = synthetic polymer
  source 1..25: mol_type = other DNA
caccgtccttcagagccaggagtcc

SEQ ID NO: 112 - DNA, 25 bp, synthetic construct
  misc_feature 1..25: note = synthetic polymer
  source 1..25: mol_type = other DNA
caccgtggtttccgagcttgaccct

SEQ ID NO: 113 - DNA, 25 bp, synthetic construct
  misc_feature 1..25: note = synthetic polymer
  source 1..25: mol_type = other DNA
caccgctgcagagtatctgctgggg

SEQ ID NO: 114 - DNA, 25 bp, synthetic construct
  misc_feature 1..25: note = synthetic polymer
  source 1..25: mol_type = other DNA
caccgcgttcctgcagagtatctgc

SEQ ID NO: 115 - DNA, 25 bp, synthetic construct
  misc_feature 1..25: note = synthetic polymer
  source 1..25: mol_type = other DNA
aaacggatctgttttcgtggctccc

SEQ ID NO: 116 - DNA, 25 bp, synthetic construct
  misc_feature 1..25: note = synthetic polymer
  source 1..25: mol_type = other DNA
aaactgtccctggatctgttttcgc

SEQ ID NO: 117 - DNA, 25 bp, synthetic construct
  misc_feature 1..25: note = synthetic polymer
  source 1..25: mol_type = other DNA
aaacagcaccgtgtccctggatctc

SEQ ID NO: 118 - DNA, 25 bp, synthetic construct
  misc_feature 1..25: note = synthetic polymer
  source 1..25: mol_type = other DNA
aaaccactgtcctagcaccgtgtcc

SEQ ID NO: 119 - DNA, 25 bp, synthetic construct
  misc_feature 1..25: note = synthetic polymer
  source 1..25: mol_type = other DNA
aaacgaggctgttgggtcattttcc

SEQ ID NO: 120 - DNA, 25 bp, synthetic construct
  misc_feature 1..25: note = synthetic polymer
  source 1..25: mol_type = other DNA
aaacagtggtcaggccggccaggcc

SEQ ID NO: 121 - DNA, 25 bp, synthetic construct
  misc_feature 1..25: note = synthetic polymer
  source 1..25: mol_type = other DNA
aaacgccaggccttcagtgctcagc

SEQ ID NO: 122 - DNA, 25 bp, synthetic construct
  misc_feature 1..25: note = synthetic polymer
  source 1..25: mol_type = other DNA
aaactcagtgctcagtggaaaccac

SEQ ID NO: 123 - DNA, 25 bp, synthetic construct
  misc_feature 1..25: note = synthetic polymer
  source 1..25: mol_type = other DNA
aaaccgaaaggactcctggctatcc

SEQ ID NO: 124 - DNA, 25 bp, synthetic construct
  misc_feature 1..25: note = synthetic polymer
  source 1..25: mol_type = other DNA
aaacagtctgagcactggaagcgcc

SEQ ID NO: 125 - DNA, 25 bp, synthetic construct
  misc_feature 1..25: note = synthetic polymer
  source 1..25: mol_type = other DNA
aaaccttccctagtctgagcactgc

SEQ ID NO: 126 - DNA, 25 bp, synthetic construct
  misc_feature 1..25: note = synthetic polymer
  source 1..25: mol_type = other DNA
aaacggctctgaaggaggaggggcc

SEQ ID NO: 127 - DNA, 25 bp, synthetic construct
  misc_feature 1..25: note = synthetic polymer
  source 1..25: mol_type = other DNA
aaacggactcctggctctgaaggac

SEQ ID NO: 128 - DNA, 25 bp, synthetic construct
  misc_feature 1..25: note = synthetic polymer
  source 1..25: mol_type = other DNA
aaacagggtcaagctcggaaaccac

SEQ ID NO: 129 - DNA, 25 bp, synthetic construct
  misc_feature 1..25: note = synthetic polymer
  source 1..25: mol_type = other DNA
aaacccccagcagatactctgcagc

SEQ ID NO: 130 - DNA, 25 bp, synthetic construct
  misc_feature 1..25: note = synthetic polymer
  source 1..25: mol_type = other DNA
aaacgcagatactctgcaggaacgc

SEQ ID NO: 131 - DNA, 20 bp, synthetic construct
  misc_feature 1..20: note = synthetic polymer
  source 1..20: mol_type = other DNA
tcccctcccagaaagacctg

SEQ ID NO: 132 - DNA, 20 bp, synthetic construct
  misc_feature 1..20: note = synthetic polymer
  source 1..20: mol_type = other DNA
tgggctccaagcaatcctgg

SEQ ID NO: 133 - DNA, 20 bp, synthetic construct
  misc_feature 1..20: note = synthetic polymer
  source 1..20: mol_type = other DNA
gtggctcaggaggtacctgg

SEQ ID NO: 134 - DNA, 20 bp, synthetic construct
  misc_feature 1..20: note = synthetic polymer
  source 1..20: mol_type = other DNA
gagccacgaaaacagatcca

SEQ ID NO: 135 - DNA, 20 bp, synthetic construct
  misc_feature 1..20: note = synthetic polymer
  source 1..20: mol_type = other DNA
aagtgaacggggaagggagg

SEQ ID NO: 136 - DNA, 20 bp, synthetic construct
  misc_feature 1..20: note = synthetic polymer
  source 1..20: mol_type = other DNA
gacaaaagccgaagtccagg

SEQ ID NO: 137 - DNA, 20 bp, synthetic construct
  misc_feature 1..20: note = synthetic polymer
  source 1..20: mol_type = other DNA
gtggttgataaacccacgtg

SEQ ID NO: 138 - DNA, 20 bp, synthetic construct
  misc_feature 1..20: note = synthetic polymer
  source 1..20: mol_type = other DNA
tgggaacagccacagcaggg

SEQ ID NO: 139 - DNA, 20 bp, synthetic construct
  misc_feature 1..20: note = synthetic polymer
  source 1..20: mol_type = other DNA
gcaggggaacggggatgcag

SEQ ID NO: 140 - DNA, 20 bp, synthetic construct
  misc_feature 1..20: note = synthetic polymer
  source 1..20: mol_type = other DNA
gagatggtggacgaggaagg

SEQ ID NO: 141 - DNA, 20 bp, synthetic construct
  misc_feature 1..20: note = synthetic polymer
  source 1..20: mol_type = other DNA
gagatggctccaggaaatgg

SEQ ID NO: 142 - DNA, 20 bp, synthetic construct
  misc_feature 1..20: note = synthetic polymer
  source 1..20: mol_type = other DNA
taaggaatctgcctaacagg

SEQ ID NO: 143 - DNA, 20 bp, synthetic construct
  misc_feature 1..20: note = synthetic polymer
  source 1..20: mol_type = other DNA
tcaggagactaggaaggagg

SEQ ID NO: 144 - DNA, 20 bp, synthetic construct
  misc_feature 1..20: note = synthetic polymer
  source 1..20: mol_type = other DNA
tataaggtggtcccagctcg

SEQ ID NO: 145 - DNA, 20 bp, synthetic construct
  misc_feature 1..20: note = synthetic polymer
  source 1..20: mol_type = other DNA
ctggaagatgccatgacagg

SEQ ID NO: 146 - DNA, 20 bp, synthetic construct
  misc_feature 1..20: note = synthetic polymer
  source 1..20: mol_type = other DNA
gcacagactagagaggtaag

SEQ ID NO: 147 - DNA, 20 bp, synthetic construct
  misc_feature 1..20: note = synthetic polymer
  source 1..20: mol_type = other DNA
acagactagagaggtaaggg

SEQ ID NO: 148 - DNA, 20 bp, synthetic construct
  misc_feature 1..20: note = synthetic polymer
  source 1..20: mol_type = other DNA
gagaggtgacccgaatccac

SEQ ID NO: 149 - DNA, 20 bp, synthetic construct
  misc_feature 1..20: note = synthetic polymer
  source 1..20: mol_type = other DNA
gcacaggccccagaaggaga

SEQ ID NO: 150 - DNA, 20 bp, synthetic construct
  misc_feature 1..20: note = synthetic polymer
  source 1..20: mol_type = other DNA
ccggagaggacccagacacg

SEQ ID NO: 151 - DNA, 20 bp, synthetic construct
  misc_feature 1..20: note = synthetic polymer
  source 1..20: mol_type = other DNA
gagaggacccagacacgggg

SEQ ID NO: 152 - DNA, 20 bp, synthetic construct
  misc_feature 1..20: note = synthetic polymer
  source 1..20: mol_type = other DNA
gcaacacagcagagagcaag

SEQ ID NO: 153 - DNA, 20 bp, synthetic construct
  misc_feature 1..20: note = synthetic polymer
  source 1..20: mol_type = other DNA
gaagagggagtggaggaaga

SEQ ID NO: 154 - DNA, 20 bp, synthetic construct
  misc_feature 1..20: note = synthetic polymer
  source 1..20: mol_type = other DNA
aagacggaacctgaaggagg

SEQ ID NO: 155 - DNA, 20 bp, synthetic construct
  misc_feature 1..20: note = synthetic polymer
  source 1..20: mol_type = other DNA
agaaagcggcacaggcccag

SEQ ID NO: 156 - DNA, 20 bp, synthetic construct
  misc_feature 1..20: note = synthetic polymer
  source 1..20: mol_type = other DNA
gggaaacagtgggccagagg

SEQ ID NO: 157 - DNA, 20 bp, synthetic construct
  misc_feature 1..20: note = synthetic polymer
  source 1..20: mol_type = other DNA
gtccggactcaggagagaga

SEQ ID NO: 158 - DNA, 20 bp, synthetic construct
  misc_feature 1..20: note = synthetic polymer
  source 1..20: mol_type = other DNA
ggcacagcaagggcactcgg

SEQ ID NO: 159 - DNA, 20 bp, synthetic construct
  misc_feature 1..20: note = synthetic polymer
  source 1..20: mol_type = other DNA
gaagaggggaagtcgaggga

SEQ ID NO: 160 - DNA, 20 bp, synthetic construct
  misc_feature 1..20: note = synthetic polymer
  source 1..20: mol_type = other DNA
gggaatggtaaggaggcctg

SEQ ID NO: 161 - DNA, 20 bp, synthetic construct
  misc_feature 1..20: note = synthetic polymer
  source 1..20: mol_type = other DNA
gcagagtggtcagcacagag

SEQ ID NO: 162 - DNA, 20 bp, synthetic construct
  misc_feature 1..20: note = synthetic polymer
  source 1..20: mol_type = other DNA
gcacagagtggctaagccca

SEQ ID NO: 163 - DNA, 20 bp, synthetic construct
  misc_feature 1..20: note = synthetic polymer
  source 1..20: mol_type = other DNA
gacggggtgtcagcataggg

SEQ ID NO: 164 - DNA, 20 bp, synthetic construct
  misc_feature 1..20: note = synthetic polymer
  source 1..20: mol_type = other DNA
gcccagggccaggaacgacg

SEQ ID NO: 165 - DNA, 20 bp, synthetic construct
  misc_feature 1..20: note = synthetic polymer
  source 1..20: mol_type = other DNA
ggtggagtccagcacggcgc

SEQ ID NO: 166 - DNA, 20 bp, synthetic construct
  misc_feature 1..20: note = synthetic polymer
  source 1..20: mol_type = other DNA
acaggccgccaggaactcgg

SEQ ID NO: 167 - DNA, 20 bp, synthetic construct
  misc_feature 1..20: note = synthetic polymer
  source 1..20: mol_type = other DNA
actaggaagtgtgtagcacc

SEQ ID NO: 168 - DNA, 20 bp, synthetic construct
  misc_feature 1..20: note = synthetic polymer
  source 1..20: mol_type = other DNA
atgaatagcagactgccccg

SEQ ID NO: 169 - DNA, 20 bp, synthetic construct
  misc_feature 1..20: note = synthetic polymer
  source 1..20: mol_type = other DNA
acacccctaaaagcacagtg

SEQ ID NO: 170 - DNA, 20 bp, synthetic construct
  misc_feature 1..20: note = synthetic polymer
  source 1..20: mol_type = other DNA
caaggagttccagcaggtgg

SEQ ID NO: 171 - DNA, 20 bp, synthetic construct
  misc_feature 1..20: note = synthetic polymer
  source 1..20: mol_type = other DNA
aaggagttccagcaggtggg

SEQ ID NO: 172 - DNA, 20 bp, synthetic construct
  misc_feature 1..20: note = synthetic polymer
  source 1..20: mol_type = other DNA
tggaaagaggagggaagagg

SEQ ID NO: 173 - DNA, 20 bp, synthetic construct
  misc_feature 1..20: note = synthetic polymer
  source 1..20: mol_type = other DNA
tcgaattcctaactgccccg

SEQ ID NO: 174 - DNA, 20 bp, synthetic construct
  misc_feature 1..20: note = synthetic polymer
  source 1..20: mol_type = other DNA
gacctgcccagcacaccctg

SEQ ID NO: 175 - DNA, 20 bp, synthetic construct
  misc_feature 1..20: note = synthetic polymer
  source 1..20: mol_type = other DNA
ggagcagctgcggcagtggg

SEQ ID NO: 176 - DNA, 20 bp, synthetic construct
  misc_feature 1..20: note = synthetic polymer
  source 1..20: mol_type = other DNA
gggagggagagcttggcagg

SEQ ID NO: 177 - DNA, 20 bp, synthetic construct
  misc_feature 1..20: note = synthetic polymer
  source 1..20: mol_type = other DNA
gttacgtggccaagaagcag

SEQ ID NO: 178 - DNA, 20 bp, synthetic construct
  misc_feature 1..20: note = synthetic polymer
  source 1..20: mol_type = other DNA
gctgaacagagaagagctgg

SEQ ID NO: 179 - DNA, 20 bp, synthetic construct
  misc_feature 1..20: note = synthetic polymer
  source 1..20: mol_type = other DNA
tctgagggtggagggactgg

SEQ ID NO: 180 - DNA, 20 bp, synthetic construct
  misc_feature 1..20: note = synthetic polymer
  source 1..20: mol_type = other DNA
ggagaggtgagggacttggg

SEQ ID NO: 181 - DNA, 20 bp, synthetic construct
  misc_feature 1..20: note = synthetic polymer
  source 1..20: mol_type = other DNA
gtgaaccaggcagacaacga

SEQ ID NO: 182 - DNA, 20 bp, synthetic construct
  misc_feature 1..20: note = synthetic polymer
  source 1..20: mol_type = other DNA
caggtacctcctgagccacg

SEQ ID NO: 183 - DNA, 20 bp, synthetic construct
  misc_feature 1..20: note = synthetic polymer
  source 1..20: mol_type = other DNA
gggggagtaggggcatgcag

SEQ ID NO: 184 - DNA, 20 bp, synthetic construct
  misc_feature 1..20: note = synthetic polymer
  source 1..20: mol_type = other DNA
gcaaatggccagcaagggtg

SEQ ID NO: 185 - DNA, 25 bp, synthetic construct
  misc_feature 1..25: note = synthetic polymer
  source 1..25: mol_type = other DNA
caccgaatcgagaagcgactcgaca

SEQ ID NO: 186 - DNA, 25 bp, synthetic construct
  misc_feature 1..25: note = synthetic polymer
  source 1..25: mol_type = other DNA
caccggtccctgggcgttgccctgc

SEQ ID NO: 187 - DNA, 25 bp, synthetic construct
  misc_feature 1..25: note = synthetic polymer
  source 1..25: mol_type = other DNA
caccgccctgggcgttgccctgcag

SEQ ID NO: 188 - DNA, 25 bp, synthetic construct
  misc_feature 1..25: note = synthetic polymer
  source 1..25: mol_type = other DNA
caccgccgtgggaagataaactaat

SEQ ID NO: 189 - DNA, 25 bp, synthetic construct
  misc_feature 1..25: note = synthetic polymer
  source 1..25: mol_type = other DNA
caccgtcccctgcagggcaacgccc

SEQ ID NO: 190 - DNA, 25 bp, synthetic construct
  misc_feature 1..25: note = synthetic polymer
  source 1..25: mol_type = other DNA
caccggtcgagtcgcttctcgatta

SEQ ID NO: 191 - DNA, 25 bp, synthetic construct
  misc_feature 1..25: note = synthetic polymer
  source 1..25: mol_type = other DNA
caccgctgctgcctcccgtcttgta

SEQ ID NO: 192 - DNA, 25 bp, synthetic construct
  misc_feature 1..25: note = synthetic polymer
  source 1..25: mol_type = other DNA
caccggagtgccgcaatacctttat

SEQ ID NO: 193 - DNA, 25 bp, synthetic construct
  misc_feature 1..25: note = synthetic polymer
  source 1..25: mol_type = other DNA
caccgacactttggtggtgcagcaa

SEQ ID NO: 194 - DNA, 25 bp, synthetic construct
  misc_feature 1..25: note = synthetic polymer
  source 1..25: mol_type = other DNA
caccgtctcaaatggtataaaactc

SEQ ID NO: 195 - DNA, 25 bp, synthetic construct
  misc_feature 1..25: note = synthetic polymer
  source 1..25: mol_type = other DNA
caccgaatcccgcccataatcgaga

SEQ ID NO: 196 - DNA, 25 bp, synthetic construct
  misc_feature 1..25: note = synthetic polymer
  source 1..25: mol_type = other DNA
caccgtcccgcccataatcgagaag

SEQ ID NO: 197 - DNA, 25 bp, synthetic construct
  misc_feature 1..25: note = synthetic polymer
  source 1..25: mol_type = other DNA
caccgcccataatcgagaagcgact

SEQ ID NO: 198 - DNA, 25 bp, synthetic construct
  misc_feature 1..25: note = synthetic polymer
  source 1..25: mol_type = other DNA
caccggagaagcgactcgacatgga

SEQ ID NO: 199 - DNA, 25 bp, synthetic construct
  misc_feature 1..25: note = synthetic polymer
  source 1..25: mol_type = other DNA
caccggaagcgactcgacatggagg

SEQ ID NO: 200 - DNA, 25 bp, synthetic construct
  misc_feature 1..25: note = synthetic polymer
  source 1..25: mol_type = other DNA
caccggcgactcgacatggaggcga

SEQ ID NO: 201 - DNA, 25 bp, synthetic construct
  misc_feature 1..25: note = synthetic polymer
  source 1..25: mol_type = other DNA
aaactgtcgagtcgcttctcgattc

SEQ ID NO: 202 - DNA, 25 bp, synthetic construct
  misc_feature 1..25: note = synthetic polymer
  source 1..25: mol_type = other DNA
aaacgcagggcaacgcccagggacc

SEQ ID NO: 203 - DNA, 25 bp, synthetic construct
  misc_feature 1..25: note = synthetic polymer
  source 1..25: mol_type = other DNA
aaacctgcagggcaacgcccagggc

SEQ ID NO: 204 - DNA, 25 bp, synthetic construct
  misc_feature 1..25: note = synthetic polymer
  source 1..25: mol_type = other DNA
aaacattagtttatcttcccacggc

SEQ ID NO: 205 - DNA, 25 bp, synthetic construct
  misc_feature 1..25: note = synthetic polymer
  source 1..25: mol_type = other DNA
aaacgggcgttgccctgcaggggac

SEQ ID NO: 206 - DNA, 25 bp, synthetic construct
  misc_feature 1..25: note = synthetic polymer
  source 1..25: mol_type = other DNA
aaactaatcgagaagcgactcgacc

SEQ ID NO: 207 - DNA, 25 bp, synthetic construct
  misc_feature 1..25: note = synthetic polymer
  source 1..25: mol_type = other DNA
aaactacaagacgggaggcagcagc

SEQ ID NO: 208 - DNA, 25 bp, synthetic construct
  misc_feature 1..25: note = synthetic polymer
  source 1..25: mol_type = other DNA
aaacataaaggtattgcggcactcc

SEQ ID NO: 209 - DNA, 25 bp, synthetic construct
  misc_feature 1..25: note = synthetic polymer
  source 1..25: mol_type = other DNA
aaacttgctgcaccaccaaagtgtc

SEQ ID NO: 210 - DNA, 25 bp, synthetic construct
  misc_feature 1..25: note = synthetic polymer
  source 1..25: mol_type = other DNA
aaacgagttttataccatttgagac

SEQ ID NO: 211 - DNA, 25 bp, synthetic construct
  misc_feature 1..25: note = synthetic polymer
  source 1..25: mol_type = other DNA
aaactctcgattatgggcgggattc

SEQ ID NO: 212 - DNA, 25 bp, synthetic construct
  misc_feature 1..25: note = synthetic polymer
  source 1..25: mol_type = other DNA
aaaccttctcgattatgggcgggac

SEQ ID NO: 213 - DNA, 25 bp, synthetic construct
  misc_feature 1..25: note = synthetic polymer
  source 1..25: mol_type = other DNA
aaacagtcgcttctcgattatgggc

SEQ ID NO: 214 - DNA, 25 bp, synthetic construct
  misc_feature 1..25: note = synthetic polymer
  source 1..25: mol_type = other DNA
aaactccatgtcgagtcgcttctcc

SEQ ID NO: 215 - DNA, 25 bp, synthetic construct
  misc_feature 1..25: note = synthetic polymer
  source 1..25: mol_type = other DNA
aaaccctccatgtcgagtcgcttcc

SEQ ID NO: 216 - DNA, 25 bp, synthetic construct
  misc_feature 1..25: note = synthetic polymer
  source 1..25: mol_type = other DNA
aaactcgcctccatgtcgagtcgcc

SEQ ID NO: 217 - DNA, 25 bp, synthetic construct
  misc_feature 1..25: note = synthetic polymer
  source 1..25: mol_type = other DNA
caccgacagggttaatgtgaagtcc

SEQ ID NO: 218 - DNA, 25 bp, synthetic construct
  misc_feature 1..25: note = synthetic polymer
  source 1..25: mol_type = other DNA
caccgtccccctctacatttaaagt

SEQ ID NO: 219 - DNA, 25 bp, synthetic construct
  misc_feature 1..25: note = synthetic polymer
  source 1..25: mol_type = other DNA
caccgcatttaaagttggtttaagt

SEQ ID NO: 220 - DNA, 25 bp, synthetic construct
  misc_feature 1..25: note = synthetic polymer
  source 1..25: mol_type = other DNA
caccgttagaaaatataaagaataa

SEQ ID NO: 221 - DNA, 25 bp, synthetic construct
  misc_feature 1..25: note = synthetic polymer
  source 1..25: mol_type = other DNA
caccgtaaatgcttactggtttgaa

SEQ ID NO: 222 - DNA, 25 bp, synthetic construct
  misc_feature 1..25: note = synthetic polymer
  source 1..25: mol_type = other DNA
caccgtcctgggtccagaaaaagat

SEQ ID NO: 223 - DNA, 25 bp, synthetic construct
  misc_feature 1..25: note = synthetic polymer
  source 1..25: mol_type = other DNA
caccgttgggtggtgagcatctgtg

SEQ ID NO: 224 - DNA, 25 bp, synthetic construct
  misc_feature 1..25: note = synthetic polymer
  source 1..25: mol_type = other DNA
caccgcggggagagtggagaaaaag

SEQ ID NO: 225 - DNA, 25 bp, synthetic construct
  misc_feature 1..25: note = synthetic polymer
  source 1..25: mol_type = other DNA
caccggttaaaactctttagacaac

SEQ ID NO: 226 - DNA, 25 bp, synthetic construct
  misc_feature 1..25: note = synthetic polymer
  source 1..25: mol_type = other DNA
caccggaaaatccccactaagatcc

SEQ ID NO: 227 - DNA, 25 bp, synthetic construct
  misc_feature 1..25: note = synthetic polymer
  source 1..25: mol_type = other DNA
aaacggacttcacattaaccctgtc

SEQ ID NO: 228 - DNA, 25 bp, synthetic construct
  misc_feature 1..25: note = synthetic polymer
  source 1..25: mol_type = other DNA
aaacactttaaatgtagagggggac

SEQ ID NO: 229 - DNA, 25 bp, synthetic construct
  misc_feature 1..25: note = synthetic polymer
  source 1..25: mol_type = other DNA
aaacacttaaaccaactttaaatgc

SEQ ID NO: 230 - DNA, 25 bp, synthetic construct
  misc_feature 1..25: note = synthetic polymer
  source 1..25: mol_type = other DNA
aaacttattctttatattttctaac

SEQ ID NO: 231 - DNA, 25 bp, synthetic construct
  misc_feature 1..25: note = synthetic polymer
  source 1..25: mol_type = other DNA
aaacttcaaaccagtaagcatttac

SEQ ID NO: 232 - DNA, 25 bp, synthetic construct
  misc_feature 1..25: note = synthetic polymer
  source 1..25: mol_type = other DNA
aaacatctttttctggacccaggac

SEQ ID NO: 233 - DNA, 25 bp, synthetic construct
  misc_feature 1..25: note = synthetic polymer
  source 1..25: mol_type = other DNA
aaaccacagatgctcaccacccaac

SEQ ID NO: 234 - DNA, 25 bp, synthetic construct
  misc_feature 1..25: note = synthetic polymer
  source 1..25: mol_type = other DNA
aaacctttttctccactctccccgc

SEQ ID NO: 235 - DNA, 25 bp, synthetic construct
  misc_feature 1..25: note = synthetic polymer
  source 1..25: mol_type = other DNA
aaacgttgtctaaagagttttaacc

SEQ ID NO: 236 - DNA, 25 bp, synthetic construct
  misc_feature 1..25: note = synthetic polymer
  source 1..25: mol_type = other DNA
aaacggatcttagtggggattttcc

SEQ ID NO: 237 - DNA, 20 bp, synthetic construct
  misc_feature 1..20: note = synthetic polymer
  source 1..20: mol_type = other DNA
agtagcagtaatgaagctgg

SEQ ID NO: 238 - DNA, 20 bp, synthetic construct
  misc_feature 1..20: note = synthetic polymer
  source 1..20: mol_type = other DNA
atacccagacgagaaagctg

SEQ ID NO: 239 - DNA, 20 bp, synthetic construct
  misc_feature 1..20: note = synthetic polymer
  source 1..20: mol_type = other DNA
tacccagacgagaaagctga

SEQ ID NO: 240 - DNA, 20 bp, synthetic construct
  misc_feature 1..20: note = synthetic polymer
  source 1..20: mol_type = other DNA
ggtggtgagcatctgtgtgg

SEQ ID NO: 241 - DNA, 20 bp, synthetic construct
  misc_feature 1..20: note = synthetic polymer
  source 1..20: mol_type = other DNA
aaatgagaagaagaggcaca

SEQ ID NO: 242 - DNA, 20 bp, synthetic construct
  misc_feature 1..20: note = synthetic polymer
  source 1..20: mol_type = other DNA
cttgtggcctgggagagctg

SEQ ID NO: 243 - DNA, 20 bp, synthetic construct
  misc_feature 1..20: note = synthetic polymer
  source 1..20: mol_type = other DNA
gctgtagaaggagacagagc

SEQ ID NO: 244 - DNA, 20 bp, synthetic construct
  misc_feature 1..20: note = synthetic polymer
  source 1..20: mol_type = other DNA
gagctggttgggaagacatg

SEQ ID NO: 245 - DNA, 20 bp, synthetic construct
  misc_feature 1..20: note = synthetic polymer
  source 1..20: mol_type = other DNA
ctggttgggaagacatgggg

SEQ ID NO: 246 - DNA, 20 bp, synthetic construct
  misc_feature 1..20: note = synthetic polymer
  source 1..20: mol_type = other DNA
cgtgaggatgggaaggaggg

SEQ ID NO: 247 - DNA, 20 bp, synthetic construct
  misc_feature 1..20: note = synthetic polymer
  source 1..20: mol_type = other DNA
atgcagagtcagcagaactg

SEQ ID NO: 248 - DNA, 20 bp, synthetic construct
  misc_feature 1..20: note = synthetic polymer
  source 1..20: mol_type = other DNA
aagacatcaagcacagaagg

SEQ ID NO: 249 - DNA, 20 bp, synthetic construct
  misc_feature 1..20: note = synthetic polymer
  source 1..20: mol_type = other DNA
tcaagcacagaaggaggagg

SEQ ID NO: 250 - DNA, 20 bp, synthetic construct
  misc_feature 1..20: note = synthetic polymer
  source 1..20: mol_type = other DNA
aaccgtcaataggcaaaggg

SEQ ID NO: 251 - DNA, 20 bp, synthetic construct
  misc_feature 1..20: note = synthetic polymer
  source 1..20: mol_type = other DNA
ccgtatttcagactgaatgg

SEQ ID NO: 252 - DNA, 20 bp, synthetic construct
  misc_feature 1..20: note = synthetic polymer
  source 1..20: mol_type = other DNA
gagaggacaggtgctacagg

SEQ ID NO: 253 - DNA, 20 bp, synthetic construct
  misc_feature 1..20: note = synthetic polymer
  source 1..20: mol_type = other DNA
aaccaaggaagggcaggagg

SEQ ID NO: 254 - DNA, 20 bp, synthetic construct
  misc_feature 1..20: note = synthetic polymer
  source 1..20: mol_type = other DNA
gacctctgggtggagacaga

SEQ ID NO: 255 - DNA, 20 bp, synthetic construct
  misc_feature 1..20: note = synthetic polymer
  source 1..20: mol_type = other DNA
cagatgaccatgacaagcag

SEQ ID NO: 256 - DNA, 20 bp, synthetic construct
  misc_feature 1..20: note = synthetic polymer
  source 1..20: mol_type = other DNA
aacaccagtgagtagagcgg

SEQ ID NO: 257 - DNA, 20 bp, synthetic construct
  misc_feature 1..20: note = synthetic polymer
  source 1..20: mol_type = other DNA
aggaccttgaagcacagaga

SEQ ID NO: 258 - DNA, 20 bp, synthetic construct
  misc_feature 1..20: note = synthetic polymer
  source 1..20: mol_type = other DNA
tacagaggcagactaaccca

SEQ ID NO: 259 - DNA, 20 bp, synthetic construct
  misc_feature 1..20: note = synthetic polymer
  source 1..20: mol_type = other DNA
acagaggcagactaacccag

SEQ ID NO: 260 - DNA, 20 bp, synthetic construct
  misc_feature 1..20: note = synthetic polymer
  source 1..20: mol_type = other DNA
taaatgacgtgctagacctg

SEQ ID NO: 261 - DNA, 20 bp, synthetic construct
  misc_feature 1..20: note = synthetic polymer
  source 1..20: mol_type = other DNA
agtaaccactcaggacaggg

SEQ ID NO: 262 - DNA, 20 bp, synthetic construct
  misc_feature 1..20: note = synthetic polymer
  source 1..20: mol_type = other DNA
accacaaaacagaaacacca

SEQ ID NO: 263 - DNA, 20 bp, synthetic construct
  misc_feature 1..20: note = synthetic polymer
  source 1..20: mol_type = other DNA
gtttgaagacaagcctgagg

SEQ ID NO: 264 - DNA, 20 bp, synthetic construct
  misc_feature 1..20: note = synthetic polymer
  source 1..20: mol_type = other DNA
gctgaaccccaaaagacagg

SEQ ID NO: 265 - DNA, 20 bp, synthetic construct
  misc_feature 1..20: note = synthetic polymer
  source 1..20: mol_type = other DNA
gcagctgagacacacaccag

SEQ ID NO: 266 - DNA, 20 bp, synthetic construct
  misc_feature 1..20: note = synthetic polymer
  source 1..20: mol_type = other DNA
aggacaccccaaagaagctg

SEQ ID NO: 267 - DNA, 20 bp, synthetic construct
  misc_feature 1..20: note = synthetic polymer
  source 1..20: mol_type = other DNA
ggacaccccaaagaagctga

SEQ ID NO: 268 - DNA, 20 bp, synthetic construct
  misc_feature 1..20: note = synthetic polymer
  source 1..20: mol_type = other DNA
ccagtgcaatggacagaaga

SEQ ID NO: 269 - DNA, 20 bp, synthetic construct
  misc_feature 1..20: note = synthetic polymer
  source 1..20: mol_type = other DNA
agaagagggagcctgcaagt

SEQ ID NO: 270 - DNA, 20 bp, synthetic construct
  misc_feature 1..20: note = synthetic polymer
  source 1..20: mol_type = other DNA
gtgtttgggccctagagcga

SEQ ID NO: 271 - DNA, 20 bp, synthetic construct
  misc_feature 1..20: note = synthetic polymer
  source 1..20: mol_type = other DNA
catgtgcctggtgcaatgca

SEQ ID NO: 272 - DNA, 20 bp, synthetic construct
  misc_feature 1..20: note = synthetic polymer
  source 1..20: mol_type = other DNA
tacaaagaggaagataagtg

SEQ ID NO: 273 - DNA, 20 bp, synthetic construct
  misc_feature 1..20: note = synthetic polymer
  source 1..20: mol_type = other DNA
gtcacagaatacaccactag

SEQ ID NO: 274 - DNA, 20 bp, synthetic construct
  misc_feature 1..20: note = synthetic polymer
  source 1..20: mol_type = other DNA
gggttaccctggacatggaa

SEQ ID NO: 275 - DNA, 20 bp, synthetic construct
  misc_feature 1..20: note = synthetic polymer
  source 1..20: mol_type = other DNA
catggaagggtattcactcg

SEQ ID NO: 276 - DNA, 20 bp, synthetic construct
  misc_feature 1..20: note = synthetic polymer
  source 1..20: mol_type = other DNA
agagtggcctagacaggctg

SEQ ID NO: 277 - DNA, 20 bp, synthetic construct
  misc_feature 1..20: note = synthetic polymer
  source 1..20: mol_type = other DNA
catgctggacagctcggcag

SEQ ID NO: 278 - DNA, 20 bp, synthetic construct
  misc_feature 1..20: note = synthetic polymer
  source 1..20: mol_type = other DNA
agtgaaagaagagaaaattc

SEQ ID NO: 279 - DNA, 20 bp, synthetic construct
  misc_feature 1..20: note = synthetic polymer
  source 1..20: mol_type = other DNA
tggtaagtctaagaaaccta

SEQ ID NO: 280 - DNA, 20 bp, synthetic construct
  misc_feature 1..20: note = synthetic polymer
  source 1..20: mol_type = other DNA
cccacagcctaaccacccta

SEQ ID NO: 281 - DNA, 20 bp, synthetic construct
  misc_feature 1..20: note = synthetic polymer
  source 1..20: mol_type = other DNA
aatatttcaaagccctaggg

SEQ ID NO: 282 - DNA, 20 bp, synthetic construct
  misc_feature 1..20: note = synthetic polymer
  source 1..20: mol_type = other DNA
gcactcggaacagggtctgg

SEQ ID NO: 283 - DNA, 20 bp, synthetic construct
  misc_feature 1..20: note = synthetic polymer
  source 1..20: mol_type = other DNA
agataggagctccaacagtg

SEQ ID NO: 284 - DNA, 20 bp, synthetic construct
  misc_feature 1..20: note = synthetic polymer
  source 1..20: mol_type = other DNA
aagttagagcagccaggaaa

SEQ ID NO: 285 - DNA, 20 bp, synthetic construct
  misc_feature 1..20: note = synthetic polymer
  source 1..20: mol_type = other DNA
tagagcagccaggaaaggga

SEQ ID NO: 286 - DNA, 20 bp, synthetic construct
  misc_feature 1..20: note = synthetic polymer
  source 1..20: mol_type = other DNA
tgaatacccttccatgtcca

SEQ ID NO: 287 - DNA, 20 bp, synthetic construct
  misc_feature 1..20: note = synthetic polymer
  source 1..20: mol_type = other DNA
cctgcattgcaccaggcaca

SEQ ID NO: 288 - DNA, 20 bp, synthetic construct
  misc_feature 1..20: note = synthetic polymer
  source 1..20: mol_type = other DNA
tctagggcccaaacacacct

SEQ ID NO: 289 - DNA, 20 bp, synthetic construct
  misc_feature 1..20: note = synthetic polymer
  source 1..20: mol_type = other DNA
tccctccatctatcaaaagg

SEQ ID NO: 290 - DNA, 20 bp, synthetic construct
  misc_feature 1..20: note = synthetic polymer
  source 1..20: mol_type = other DNA
agccctgagacagaagcagg

SEQ ID NO: 291 - DNA, 20 bp, synthetic construct
  misc_feature 1..20: note = synthetic polymer
  source 1..20: mol_type = other DNA
gccctgagacagaagcaggt

SEQ ID NO: 292 - DNA, 20 bp, synthetic construct
  misc_feature 1..20: note = synthetic polymer
  source 1..20: mol_type = other DNA
aggagatgcagtgatacgca

SEQ ID NO: 293 - DNA, 20 bp, synthetic construct
  misc_feature 1..20: note = synthetic polymer
  source 1..20: mol_type = other DNA
acaataccaagggtatccgg

SEQ ID NO: 294 - DNA, 20 bp, synthetic construct
  misc_feature 1..20: note = synthetic polymer
  source 1..20: mol_type = other DNA
tgataaagaaaacaaagtga

SEQ ID NO: 295 - DNA, 20 bp, synthetic construct
  misc_feature 1..20: note = synthetic polymer
  source 1..20: mol_type = other DNA
aaagaaaacaaagtgaggga

SEQ ID NO: 296 - DNA, 20 bp, synthetic construct
  misc_feature 1..20: note = synthetic polymer
  source 1..20: mol_type = other DNA
gtggcaagtggagaaattga

SEQ ID NO: 297 - DNA, 20 bp, synthetic construct
  misc_feature 1..20: note = synthetic polymer
  source 1..20: mol_type = other DNA
caagtggagaaattgaggga

SEQ ID NO: 298 - DNA, 20 bp, synthetic construct
  misc_feature 1..20: note = synthetic polymer
  source 1..20: mol_type = other DNA
gtggtgatgattgcagctgg

SEQ ID NO: 299 - DNA, 20 bp, synthetic construct
  misc_feature 1..20: note = synthetic polymer
  source 1..20: mol_type = other DNA
ctatgtgcctgacacacagg

SEQ ID NO: 300 - DNA, 20 bp, synthetic construct
  misc_feature 1..20: note = synthetic polymer
  source 1..20: mol_type = other DNA
gggttggaccaggaaagagg

SEQ ID NO: 301 - DNA, 20 bp, synthetic construct
  misc_feature 1..20: note = synthetic polymer
  source 1..20: mol_type = other DNA
gatgcctggaaaaggaaaga

SEQ ID NO: 302 - DNA, 20 bp, synthetic construct
  misc_feature 1..20: note = synthetic polymer
  source 1..20: mol_type = other DNA
tagtatgcacctgcaagagg

SEQ ID NO: 303 - DNA, 20 bp, synthetic construct
  misc_feature 1..20: note = synthetic polymer
  source 1..20: mol_type = other DNA
tatgcacctgcaagaggcgg

SEQ ID NO: 304 - DNA, 20 bp, synthetic construct
  misc_feature 1..20: note = synthetic polymer
  source 1..20: mol_type = other DNA
aggggaagaagagaagcaga

SEQ ID NO: 305 - DNA, 20 bp, synthetic construct
  misc_feature 1..20: note = synthetic polymer
  source 1..20: mol_type = other DNA
gctgaatcaagagacaagcg

SEQ ID NO: 306 - DNA, 20 bp, synthetic construct
  misc_feature 1..20: note = synthetic polymer
  source 1..20: mol_type = other DNA
aagcaaataaatctcctggg

SEQ ID NO: 307 - DNA, 20 bp, synthetic construct
  misc_feature 1..20: note = synthetic polymer
  source 1..20: mol_type = other DNA
agatgagtgctagagactgg

SEQ ID NO: 308 - DNA, 20 bp, synthetic construct
  misc_feature 1..20: note = synthetic polymer
  source 1..20: mol_type = other DNA
ctgatggttgagcacagcag

SEQ ID NO: 309 - DNA, 20 bp, synthetic construct
  misc_feature 1..20: note = synthetic polymer
  source 1..20: mol_type = other DNA
caaatggccagcaagggtgg

SEQ ID NO: 310 - DNA, 20 bp, synthetic construct
  misc_feature 1..20: note = synthetic polymer
  source 1..20: mol_type = other DNA
gcagaacctgaggatatgga

SEQ ID NO: 311 - DNA, 20 bp, synthetic construct
  misc_feature 1..20: note = synthetic polymer
  source 1..20: mol_type = other DNA
aatacacagaatgaaaatag

SEQ ID NO: 312 - DNA, 20 bp, synthetic construct
  misc_feature 1..20: note = synthetic polymer
  source 1..20: mol_type = other DNA
ctggtgactagaataggcag

SEQ ID NO: 313 - DNA, 20 bp, synthetic construct
  misc_feature 1..20: note = synthetic polymer
  source 1..20: mol_type = other DNA
tggtgactagaataggcagt

SEQ ID NO: 314 - DNA, 20 bp, synthetic construct
  misc_feature 1..20: note = synthetic polymer
  source 1..20: mol_type = other DNA
taaaagaatgtgaaaagatg

SEQ ID NO: 315 - DNA, 20 bp, synthetic construct
  misc_feature 1..20: note = synthetic polymer
  source 1..20: mol_type = other DNA
tcaggagttcaagaccaccc

SEQ ID NO: 316 - DNA, 20 bp, synthetic construct
  misc_feature 1..20: note = synthetic polymer
  source 1..20: mol_type = other DNA
tgtagtcccagttatgcagg

SEQ ID NO: 317 - DNA, 20 bp, synthetic construct
  misc_feature 1..20: note = synthetic polymer
  source 1..20: mol_type = other DNA
gggttcacaccacaaatgca

SEQ ID NO: 318 - DNA, 20 bp, synthetic construct
  misc_feature 1..20: note = synthetic polymer
  source 1..20: mol_type = other DNA
ggcaaatggccagcaagggt

SEQ ID NO: 319 - DNA, 20 bp, synthetic construct
  misc_feature 1..20: note = synthetic polymer
  source 1..20: mol_type = other DNA
agaaaccaatcccaaagcaa

SEQ ID NO: 320 - DNA, 20 bp, synthetic construct
  misc_feature 1..20: note = synthetic polymer
  source 1..20: mol_type = other DNA
gccaaggacaccaaaaccca

SEQ ID NO: 321 - DNA, 20 bp, synthetic construct
  misc_feature 1..20: note = synthetic polymer
  source 1..20: mol_type = other DNA
agtggtgataaggcaacagt

SEQ ID NO: 322 - DNA, 20 bp, synthetic construct
  misc_feature 1..20: note = synthetic polymer
  source 1..20: mol_type = other DNA
cctgagacagaagtattaag

SEQ ID NO: 323 - DNA, 20 bp, synthetic construct
  misc_feature 1..20: note = synthetic polymer
  source 1..20: mol_type = other DNA
aaggtcacacaatgaatagg

SEQ ID NO: 324 - DNA, 20 bp, synthetic construct
  misc_feature 1..20: note = synthetic polymer
  source 1..20: mol_type = other DNA
caccatactagggaagaaga

SEQ ID NO: 325 - DNA, 20 bp, synthetic construct
  misc_feature 1..20: note = synthetic polymer
  source 1..20: mol_type = other DNA
aataccctgcccttagtggg

SEQ ID NO: 326 - DNA, 20 bp, synthetic construct
  misc_feature 1..20: note = synthetic polymer
  source 1..20: mol_type = other DNA
ttagtggggggtggagtggg

SEQ ID NO: 327 - DNA, 20 bp, synthetic construct
  misc_feature 1..20: note = synthetic polymer
  source 1..20: mol_type = other DNA
caataccctgcccttagtgg

SEQ ID NO: 328 - DNA, 20 bp, synthetic construct
  misc_feature 1..20: note = synthetic polymer
  source 1..20: mol_type = other DNA
gtggggggtggagtgggggg

SEQ ID NO: 329 - DNA, 20 bp, synthetic construct
  misc_feature 1..20: note = synthetic polymer
  source 1..20: mol_type = other DNA
ggggggtggagtggggggtg

SEQ ID NO: 330 - DNA, 20 bp, synthetic construct
  misc_feature 1..20: note = synthetic polymer
  source 1..20: mol_type = other DNA
ggggtggagtggggggtggg

SEQ ID NO: 331 - DNA, 20 bp, synthetic construct
  misc_feature 1..20: note = synthetic polymer
  source 1..20: mol_type = other DNA
gggtggagtggggggtgggg

SEQ ID NO: 332 - DNA, 20 bp, synthetic construct
  misc_feature 1..20: note = synthetic polymer
  source 1..20: mol_type = other DNA
gggggtggggaaagacatcg

SEQ ID NO: 333 - DNA, 20 bp, synthetic construct
  misc_feature 1..20: note = synthetic polymer
  source 1..20: mol_type = other DNA
gcagctgtgaattctgatag

SEQ ID NO: 334 - DNA, 20 bp, synthetic construct
  misc_feature 1..20: note = synthetic polymer
  source 1..20: mol_type = other DNA
gagatcagagaaaccagatg

SEQ ID NO: 335 - DNA, 20 bp, synthetic construct
  misc_feature 1..20: note = synthetic polymer
  source 1..20: mol_type = other DNA
tctatactgattgcagccag

SEQ ID NO: 336-347 - skipped (12 entries)

SEQ ID NO: 348 - protein, 6 aa, synthetic construct
  REGION 1..6: note = synthetic polymer
  VARIANT 4: note = X can be any naturally occurring amino acid
KKRXKR

SEQ ID NO: 349 - protein, 19 aa, synthetic construct
  REGION 1..19: note = synthetic polymer
  VARIANT 1: note = K may be replaced with R
  VARIANT 2: note = K may be replaced with R
  VARIANT 13: note = position may be deleted
  VARIANT 14: note = position may be deleted
  VARIANT 15..19: note = at least 3 of the 5 amino acids are K or R
KKXXXXXXXXXXXXKKKKK

SEQ ID NO: 350 - protein, 9 aa, synthetic construct
  REGION 1..9: note = synthetic polymer
PAAKRVKLD

SEQ ID NO: 351 - protein, 16 aa, synthetic construct
  REGION 1..16: note = synthetic polymer
KRPAATKKAGQAKKKK

SEQ ID NO: 352 - protein, 7 aa, synthetic construct
  REGION 1..7: note = synthetic polymer
PKKKRKV

SEQ ID NO: 353 - protein, 24 aa, synthetic construct
  REGION 1..24: note = synthetic polymer
DPKKKRKVDPKKKRKVDPKKKRKV

SEQ ID NO: 354-422 - skipped (69 entries)

SEQ ID NO: 423 - DNA, 333 bp, synthetic construct
  misc_feature 1..333: note = synthetic polymer
  source 1..333: mol_type = other DNA
ggcggatctggcggtagtgctgagtattgtctgagttacgaaacggaaatactcacggttgagtatgggcttcttccaattggcaaaatcgttgaaaagcgcatagagtgtacggtgtattccgtcgataacaacggtaatatctacacccagccggtagctcagtggcacgaccgaggcgaacaggaagtgttcgagtattgcttggaagatggctcccttatccgcgccactaaagaccataagtttatgacggttgacgggcagatgctgcctatagacgaaatatttgagagagagctggacttgatgagagtcgataatctgccaaat

SEQ ID NO: 424 - DNA, 180 bp, synthetic construct
  misc_feature 1..180: note = synthetic polymer
  source 1..180: mol_type = other DNA
ggcggatctggcggtagtgggggttccggatccataaagatagctactaggaaatatcttggcaaacaaaacgtctatgacataggagttgagcgagatcacaattttgctttgaagaatgggttcatcgcgtctaattgcttcaacgctagcggcgggtcaggaggctctggtggaagc

SEQ ID NO: 425 - DNA, 20 bp, synthetic construct
  misc_feature 1..20: note = synthetic polymer
  source 1..20: mol_type = other DNA
aatcgagaagcgactcgaca

SEQ ID NO: 426 - DNA, 20 bp, synthetic construct
  misc_feature 1..20: note = synthetic polymer
  source 1..20: mol_type = other DNA
tgccctgcaggggagtgagc

SEQ ID NO: 427 - DNA, 20 bp, synthetic construct
  misc_feature 1..20: note = synthetic polymer
  source 1..20: mol_type = other DNA
gaagcgactcgacatggagg

SEQ ID NO: 428 - DNA, 20 bp, synthetic construct
  misc_feature 1..20: note = synthetic polymer
  source 1..20: mol_type = other DNA
cctgcaggggagtgagcagc

SEQ ID NO: 429-502 - skipped (74 entries)

SEQ ID NO: 503 - DNA, 1614 bp, synthetic construct
  misc_feature 1..1614: note = synthetic polymer
  source 1..1614: mol_type = other DNA
atgagctctgacgacgaggtgatggtgcggcccagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattac

SEQ ID NO: 504 - protein, 538 aa, synthetic construct
MSSDDEVMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNY

SEQ ID NO: 505 - DNA, 1584 bp, synthetic construct
  misc_feature 1..1584: note = synthetic polymer
  source 1..1584: mol_type = other DNA
atgagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattac

SEQ ID NO: 506 - protein, 528 aa, synthetic construct
MRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNY

SEQ ID NO: 507 - DNA, 1515 bp, synthetic construct
  misc_feature 1..1515: note = synthetic polymer
  source 1..1515: mol_type = other DNA
atggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattac

SEQ ID NO: 508 - protein, 505 aa, synthetic construct
MEEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNY

SEQ ID NO: 509 - DNA, 1452 bp, synthetic construct
  misc_feature 1..1452: note = synthetic polymer
  source 1..1452: mol_type = other DNA
atgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattac

SEQ ID NO: 510 - protein, 484 aa, synthetic construct
MNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCNIPLHKGACFEKYHTLKNY

SEQ ID NO: 511 - DNA, 1662 bp, synthetic construct
  misc_feature 1..1662: note = synthetic polymer
  source 1..1662: mol_type = other DNA
atggcccagcacagcgactaccccgacgacgagttcagagccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaac

SEQ ID NO: 512 - protein, 554 aa, synthetic construct
MAQHSDYPDDEFRADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKNILRRCRVCSVHKLRSETRYMCKFCN

SEQ ID NO: 513 - DNA, 1587 bp, synthetic construct
  misc_feature 1..1587: note = synthetic polymer
  source 1..1587: mol_type = other DNA
atggcccagcacagcgactaccccgacgacgagttcagagccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaac

SEQ ID NO: 514 - protein, 529 aa, synthetic construct
MAQHSDYPDDEFRADKLSNYSCDSDLENASTSDEDSSDDEVMVRPRTLRRRRISSSSSDSESDIEGGREEWSHVDNPPVLEDFLGHQGLNTDAVINNIEDAVKLFIGDDFFEFLVEESNRYYNQNRNNFKLSKKSLKWKDITPQEMKKFLGLIVLMGQVRKDRRDDYWTTEPWTETPYFGKTMTRDRFRQIWKAWHFNNNADIVNESDRLCKVRPVLDYFVPKFINIYKPHQQLSLDEGIVPWRGRLFFRVYNAGKIVKYGILVRLLCESDTGYICNMEIYCGEGKRLLETIQTVVSPYTDSWYHIYMDNYYNSVANCEALMKNKFRICGTIRKNRGIPKDFQTISLKKGETKFIRKNDILLQVWQSKKPVYLISSIHSAEMEESQNIDRTSKKKIVKPNALIDYNKHMKGVDRADQYLSYYSILRRTVKWTKRLAMYMINCALFNSYAVYKSVRQRKMGFKMFLKQTAIHWLTDDIPEDMDIVPDLQPVPSTSGMRAKPPTSDPPCRLSMDMRKHTLQAIVGSGKKKN

SEQ ID NO: 515-798 - skipped (284 entries)

SEQ ID NO: 799 - DNA, 20 bp, synthetic construct
  source 1..20: mol_type = other DNA
gagtgagcagctgtaagatt

SEQ ID NO: 800 - DNA, 20 bp, synthetic construct
  source 1..20: mol_type = other DNA
caggggagtgagcagctgta

SEQ ID NO: 801 - DNA, 20 bp, synthetic construct
  source 1..20: mol_type = other DNA
cctgcaggggagtgagcagc

SEQ ID NO: 802 - DNA, 20 bp, synthetic construct
  source 1..20: mol_type = other DNA
tgccctgcaggggagtgagc

SEQ ID NO: 803 - DNA, 20 bp, synthetic construct
  source 1..20: mol_type = other DNA
cgttgccctgcaggggagtg

SEQ ID NO: 804 - DNA, 20 bp, synthetic construct
  source 1..20: mol_type = other DNA
tgggcgttgccctgcagggg

SEQ ID NO: 805 - DNA, 20 bp, synthetic construct
  source 1..20: mol_type = other DNA
ttggtccctgggcgttgccc

SEQ ID NO: 806 - DNA, 20 bp, synthetic construct
  source 1..20: mol_type = other DNA
aagaatcccgcccataatcg

SEQ ID NO: 807 - DNA, 20 bp, synthetic construct
  source 1..20: mol_type = other DNA
aatcccgcccataatcgaga

SEQ ID NO: 808 - DNA, 20 bp, synthetic construct
  source 1..20: mol_type = other DNA
tcccgcccataatcgagaag

SEQ ID NO: 809 - DNA, 20 bp, synthetic construct
  source 1..20: mol_type = other DNA
cccataatcgagaagcgact

SEQ ID NO: 810 - DNA, 20 bp, synthetic construct
  source 1..20: mol_type = other DNA
gagaagcgactcgacatgga

SEQ ID NO: 811 - DNA, 20 bp, synthetic construct
  source 1..20: mol_type = other DNA
gaagcgactcgacatggagg

SEQ ID NO: 812 - DNA, 20 bp, synthetic construct
  source 1..20: mol_type = other DNA
gcgactcgacatggaggcga

SEQ ID NO: 813 - DNA, 20 bp, synthetic construct
  source 1..20: mol_type = other DNA
ccgtgggaagataaactaat

SEQ ID NO: 814 - DNA, 20 bp, synthetic construct
  source 1..20: mol_type = other DNA
tcccctgcagggcaacgccc

SEQ ID NO: 815 - DNA, 20 bp, synthetic construct
  source 1..20: mol_type = other DNA
gtcgagtcgcttctcgatta

SEQ ID NO: 816 - DNA, 20 bp, synthetic construct
  source 1..20: mol_type = other DNA
cgacaccaactctagtccgt

SEQ ID NO: 817 - DNA, 20 bp, synthetic construct
  source 1..20: mol_type = other DNA
cagctgctcactcccctgca

SEQ ID NO: 818 - DNA, 20 bp, synthetic construct
  source 1..20: mol_type = other DNA
agtcgcttctcgattatggg

SEQ ID NO: 819 - DNA, 23 bp, synthetic construct
  source 1..23: mol_type = other DNA
acccttggaaggacctggctggg

SEQ ID NO: 820 - DNA, 20 bp, synthetic construct
  source 1..20: mol_type = other DNA
tccgagcttgacccttggaa

SEQ ID NO: 821 - DNA, 23 bp, synthetic construct
  source 1..23: mol_type = other DNA
ggagccacgaaaacagatccagg

SEQ ID NO: 822 - DNA, 20 bp, synthetic construct
  source 1..20: mol_type = other DNA
tggtttccgagcttgaccct

SEQ ID NO: 823 - DNA, 23 bp, synthetic construct
  source 1..23: mol_type = other DNA
ggagccacgaaaacagatccagg

SEQ ID NO: 824 - DNA, 23 bp, synthetic construct
  source 1..23: mol_type = other DNA
agatccagggacacggtgctagg

SEQ ID NO: 825 - DNA, 23 bp, synthetic construct
  source 1..23: mol_type = other DNA
gacacggtgctaggacagtgggg

SEQ ID NO: 826 - DNA, 23 bp, synthetic construct
  source 1..23: mol_type = other DNA
gaaaatgacccaacagcctctgg

SEQ ID NO: 827 - DNA, 23 bp, synthetic construct
  source 1..23: mol_type = other DNA
gcctggccggcctgaccactggg

SEQ ID NO: 828 - DNA, 23 bp, synthetic construct
  source 1..23: mol_type = other DNA
ctgagcactgaaggcctggccgg

SEQ ID NO: 829 - DNA, 23 bp, synthetic construct
  source 1..23: mol_type = other DNA
tggtttccactgagcactgaagg

SEQ ID NO: 830 - DNA, 23 bp, synthetic construct
  source 1..23: mol_type = other DNA
ggtgctttcctgaggaccgatag

SEQ ID NO: 831 - DNA, 23 bp, synthetic construct
  source 1..23: mol_type = other DNA
gcgcttccagtgctcagactagg

SEQ ID NO: 832 - DNA, 23 bp, synthetic construct
  source 1..23: mol_type = other DNA
cagtgctcagactagggaagagg

SEQ ID NO: 833 - DNA, 23 bp, synthetic construct
  source 1..23: mol_type = other DNA
gcccctcctccttcagagccagg

SEQ ID NO: 834 - DNA, 23 bp, synthetic construct
  source 1..23: mol_type = other DNA
tccttcagagccaggagtcctgg

SEQ ID NO: 835 - DNA, 23 bp, synthetic construct
  source 1..23: mol_type = other DNA
ccaagggtcaagctcggaaacca

SEQ ID NO: 836 - DNA, 23 bp, synthetic construct
  source 1..23: mol_type = other DNA
ctgcagagtatctgctggggtgg

SEQ ID NO: 837 - DNA, 23 bp, synthetic construct
  source 1..23: mol_type = other DNA
cgttcctgcagagtatctgctgg

SEQ ID NO: 838 - DNA, 20 bp, synthetic construct
  source 1..20: mol_type = other DNA
gtggggaaaatgacccaaca

SEQ ID NO: 839 - DNA, 20 bp, synthetic construct
  source 1..20: mol_type = other DNA
gaaggcctggccggcctgac

SEQ ID NO: 840 - DNA, 20 bp, synthetic construct
  source 1..20: mol_type = other DNA
actcctggctctgaaggagg

SEQ ID NO: 841 - DNA, 20 bp, synthetic construct
  source 1..20: mol_type = other DNA
gggctgggggccaggactcc

SEQ ID NO: 842 - DNA, 20 bp, synthetic construct
  source 1..20: mol_type = other DNA
gtccttccaagggtcaagct

SEQ ID NO: 843 - DNA, 20 bp, synthetic construct
  source 1..20: mol_type = other DNA
tcaagctcggaaaccacccc

SEQ ID NO: 844 - DNA, 23 bp, synthetic construct
  source 1..23: mol_type = other DNA
attgtcttcactaaacccgttgg

SEQ ID NO: 845 - DNA, 23 bp, synthetic construct
  source 1..23: mol_type = other DNA
taaacccgttgggaatacaatgg

SEQ ID NO: 846 - DNA, 23 bp, synthetic construct
  source 1..23: mol_type = other DNA
ttgtcttcactaaacccgttggg

SEQ ID NO: 847 - DNA, 23 bp, synthetic construct
  source 1..23: mol_type = other DNA
tgattcataggagtctattaagg

SEQ ID NO: 848 - DNA, 23 bp, synthetic construct
  source 1..23: mol_type = other DNA
ttacatatgcttcgagtttgtgg

SEQ ID NO: 849 - DNA, 23 bp, synthetic construct
  source 1..23: mol_type = other DNA
actcttaaggtaggactaattgg

SEQ ID NO: 850 - DNA, 23 bp, synthetic construct
  source 1..23: mol_type = other DNA
tatgtgtgcaatagcgttaaagg

SEQ ID NO: 851 - DNA, 23 bp, synthetic construct
  source 1..23: mol_type = other DNA
cgttgggaatacaatggcttagg

SEQ ID NO: 852 - DNA, 23 bp, synthetic construct
  source 1..23: mol_type = other DNA
tcacaatggaactctgcctttgg

SEQ ID NO: 853 - DNA, 23 bp, synthetic construct
  source 1..23: mol_type = other DNA
gaccacaaatcaatgcccaaagg

SEQ ID NO: 854 - DNA, 23 bp, synthetic construct
  source 1..23: mol_type = other DNA
ctaagccattgtattcccaacgg

SEQ ID NO: 855 - DNA, 23 bp, synthetic construct
  source 1..23: mol_type = other DNA
agcattctggagtgtcacaatgg

SEQ ID NO: 856 - DNA, 23 bp, synthetic construct
  source 1..23: mol_type = other DNA
caatagcccactttaatactagg

SEQ ID NO: 857 - DNA, 23 bp, synthetic construct
  source 1..23: mol_type = other DNA
ctttatccaagtgaatcctttgg

SEQ ID NO: 858 - DNA, 23 bp, synthetic construct
  source 1..23: mol_type = other DNA
ggcattgatttgtggtcatttgg

SEQ ID NO: 859 - DNA, 23 bp, synthetic construct
  source 1..23: mol_type = other DNA
taagccattgtattcccaacggg

SEQ ID NO: 860 - DNA, 23 bp, synthetic construct
  source 1..23: mol_type = other DNA
aatacaatcactcttaaggtagg

SEQ ID NO: 861 - DNA, 23 bp, synthetic construct
  source 1..23: mol_type = other DNA
gaagtacctttcactattttggg

SEQ ID NO: 862 - DNA, 23 bp, synthetic construct
  source 1..23: mol_type = other DNA
caagcaacaaatgacttctaagg

SEQ ID NO: 863 - DNA, 23 bp, synthetic construct
  source 1..23: mol_type = other DNA
tttgaatacaatcactcttaagg

SEQ ID NO: 864 - DNA, 23 bp, synthetic construct
  source 1..23: mol_type = other DNA
acaaacggactacgtaaacttgg

SEQ ID NO: 865 - DNA, 23 bp, synthetic construct
  source 1..23: mol_type = other DNA
acaagatgtgaacacgacgatgg

SEQ ID NO: 866 - DNA, 23 bp, synthetic construct
  source 1..23: mol_type = other DNA
gttgcaccgttgattccttcagg

SEQ ID NO: 867 - DNA, 23 bp, synthetic construct
  source 1..23: mol_type = other DNA
agtaatattgaattagggcgtgg

SEQ ID NO: 868 - DNA, 23 bp, synthetic construct
  source 1..23: mol_type = other DNA
cctgatgttggctcgacattagg

SEQ ID NO: 869 - DNA, 23 bp, synthetic construct
  source 1..23: mol_type = other DNA
ctttgttgggtcttagcttaagg

SEQ ID NO: 870 - DNA, 23 bp, synthetic construct
  source 1..23: mol_type = other DNA
tcggaacagctccttcctgaagg

SEQ ID NO: 871 - DNA, 23 bp, synthetic construct
  source 1..23: mol_type = other DNA
agtagtttctgaggtcatgttgg

SEQ ID NO: 872 - DNA, 23 bp, synthetic construct
  source 1..23: mol_type = other DNA
cttgaaaatacgatgatgtgagg

SEQ ID NO: 873 - DNA, 23 bp, synthetic construct
  source 1..23: mol_type = other DNA
gcattaatctagagagagggagg

SEQ ID NO: 874 - DNA, 23 bp, synthetic construct
  source 1..23: mol_type = other DNA
gggtcatgttagaattcatgtgg

SEQ ID NO: 875 - DNA, 23 bp, synthetic construct
  source 1..23: mol_type = other DNA
tgatgcattaatctagagagagg

SEQ ID NO: 876 - DNA, 23 bp, synthetic construct
  source 1..23: mol_type = other DNA
acatcatcgtattttcaagttgg

SEQ ID NO: 877 - DNA, 23 bp, synthetic construct
  source 1..23: mol_type = other DNA
ctagctgacaaacatgtgagtgg

SEQ ID NO: 878 - DNA, 23 bp, synthetic construct
  source 1..23: mol_type = other DNA
aacatgacccaagtgagtccagg

SEQ ID NO: 879 - DNA, 23 bp, synthetic construct
  source 1..23: mol_type = other DNA
gattccgtatttgctttgttggg

SEQ ID NO: 880 - DNA, 23 bp, synthetic construct
  source 1..23: mol_type = other DNA
tacgatgatgtgaggaaataagg

SEQ ID NO: 881 - DNA, 23 bp, synthetic construct
  source 1..23: mol_type = other DNA
gtaatatgtctaagtactgatgg

SEQ ID NO: 882 - DNA, 23 bp, synthetic construct
  source 1..23: mol_type = other DNA
gtaaagtgagctggttcattagg

SEQ ID NO: 883 - DNA, 23 bp, synthetic construct
  source 1..23: mol_type = other DNA
actagagtccttaagaagggggg

SEQ ID NO: 884 - DNA, 23 bp, synthetic construct
  source 1..23: mol_type = other DNA
ggaagacttttctctatacgagg

SEQ ID NO: 885 - DNA, 23 bp, synthetic construct
  source 1..23: mol_type = other DNA
gcattcctttcatccatggcagg

SEQ ID NO: 886 - DNA, 23 bp, synthetic construct
  source 1..23: mol_type = other DNA
gacatatggttataaaaatcagg

SEQ ID NO: 887 - DNA, 23 bp, synthetic construct
  source 1..23: mol_type = other DNA
ggagtgcagtccctgacatatgg

SEQ ID NO: 888 - DNA, 23 bp, synthetic construct
  source 1..23: mol_type = other DNA
gtgggttagggtggttaactggg

SEQ ID NO: 889 - DNA, 23 bp, synthetic construct
  source 1..23: mol_type = other DNA
aggtgcaaaaaggttgctgtggg

SEQ ID NO: 890 - DNA, 23 bp, synthetic construct
  source 1..23: mol_type = other DNA
cgtgacaaggcaaagtggcgtgg

SEQ ID NO: 891 - DNA, 23 bp, synthetic construct
  source 1..23: mol_type = other DNA
gaaggactgcccctgacgtcagg

SEQ ID NO: 892 - DNA, 23 bp, synthetic construct
  source 1..23: mol_type = other DNA
ctgcccctgacgtcaggagttgg

SEQ ID NO: 893 - DNA, 23 bp, synthetic construct
  source 1..23: mol_type = other DNA
tgtgggttagggtggttaactgg

SEQ ID NO: 894 - DNA, 23 bp, synthetic construct
  source 1..23: mol_type = other DNA
acccttttagagttttctgctgg

SEQ ID NO: 895 - DNA, 23 bp, synthetic construct
  source 1..23: mol_type = other DNA
aacttcctgccatggatgaaagg

SEQ ID NO: 896 - DNA, 23 bp, synthetic construct
  source 1..23: mol_type = other DNA
gcaaaaaggttgctgtgggttgg

SEQ ID NO: 897 - DNA, 23 bp, synthetic construct
  source 1..23: mol_type = other DNA
aatttgggggtagataggcatgg

SEQ ID NO: 898 - DNA, 23 bp, synthetic construct
  source 1..23: mol_type = other DNA
agaaaactctaaaagggtatagg

SEQ ID NO: 899 - DNA, 23 bp, synthetic construct
  source 1..23: mol_type = other DNA
attagcattcctttcatccatgg

SEQ ID NO: 900 - DNA, 23 bp, synthetic construct
  source 1..23: mol_type = other DNA
cccagcagaaaactctaaaaggg

SEQ ID NO: 901 - DNA, 23 bp, synthetic construct
  source 1..23: mol_type = other DNA
caggtgcaaaaaggttgctgtgg

SEQ ID NO: 902 - DNA, 23 bp, synthetic construct
  source 1..23: mol_type = other DNA
gcaagagatgaaattccatatgg

SEQ ID NO: 903 - DNA, 23 bp, synthetic construct
  source 1..23: mol_type = other DNA
gggctgttctaacgaagtctggg

SEQ ID NO: 904 - DNA, 23 bp, synthetic construct
  source 1..23: mol_type = other DNA
tgtccattcagcgaccctagagg

SEQ ID NO: 905 - DNA, 23 bp, synthetic construct
  source 1..23: mol_type = other DNA
ggctgttctaacgaagtctgggg

SEQ ID NO: 906 - DNA, 23 bp, synthetic construct
  source 1..23: mol_type = other DNA
gtccattcagcgaccctagaggg

SEQ ID NO: 907 - DNA, 23 bp, synthetic construct
  source 1..23: mol_type = other DNA
ggggctgttctaacgaagtctgg

SEQ ID NO: 908 - DNA, 23 bp, synthetic construct
  source 1..23: mol_type = other DNA
ggctgaatcagcatgcgaaaggg

SEQ ID NO: 909 - DNA, 23 bp, synthetic construct
  source 1..23: mol_type = other DNA
ttccaatggggggcatagcctgg

SEQ ID NO: 910 - DNA, 23 bp, synthetic construct
  source 1..23: mol_type = other DNA
taccctctagggtcgctgaatgg

SEQ ID NO: 911 - DNA, 23 bp, synthetic construct
  source 1..23: mol_type = other DNA
atcctcttgggccttataagagg

SEQ ID NO: 912 - DNA, 23 bp, synthetic construct
  source 1..23: mol_type = other DNA
ggccaggctatgccccccattgg

SEQ ID NO: 913 - DNA, 23 bp, synthetic construct
  source 1..23: mol_type = other DNA
ctagaggaccagaacaactctgg

SEQ ID NO: 914 - DNA, 23 bp, synthetic construct
  source 1..23: mol_type = other DNA
tccctcttataaggcccaagagg

SEQ ID NO: 915 - DNA, 23 bp, synthetic construct
  source 1..23: mol_type = other DNA
aggctgaatcagcatgcgaaagg

SEQ ID NO: 916 - DNA, 23 bp, synthetic construct
  source 1..23: mol_type = other DNA
ggaccagaacaactctggcctgg

SEQ ID NO: 917 - DNA, 23 bp, synthetic construct
  source 1..23: mol_type = other DNA
gggcttttatttggcccagcagg

SEQ ID NO: 918 - DNA, 23 bp, synthetic construct
  source 1..23: mol_type = other DNA
gtcgctgaatggacagactctgg

SEQ ID NO: 919 - DNA, 23 bp, synthetic construct
  source 1..23: mol_type = other DNA
ctcatgagttttaccctctaggg

SEQ ID NO: 920 - DNA, 23 bp, synthetic construct
  source 1..23: mol_type = other DNA
tcctcttgggccttataagaggg

SEQ ID NO: 921 - DNA, 23 bp, synthetic construct
  source 1..23: mol_type = other DNA
tcttgggccttataagagggagg

SEQ ID NO: 922 - DNA, 23 bp, synthetic construct
  source 1..23: mol_type = other DNA
tagaacagccccccacacagtgg

SEQ ID NO: 923 - DNA, 23 bp, synthetic construct
  source 1..23: mol_type = other DNA
gttacgttatgactaatctttgg

SEQ ID NO: 924 - DNA, 23 bp, synthetic construct
  source 1..23: mol_type = other DNA
tacgttatgactaatctttgggg

SEQ ID NO: 925 - DNA, 23 bp, synthetic construct
  source 1..23: mol_type = other DNA
ggaagtagtgttatgatgtatgg

SEQ ID NO: 926 - DNA, 23 bp, synthetic construct
  source 1..23: mol_type = other DNA
gttatgatgtatgggcataaagg

SEQ ID NO: 927 - DNA, 23 bp, synthetic construct
  source 1..23: mol_type = other DNA
gaagtagtgttatgatgtatggg

SEQ ID NO: 928 - DNA, 23 bp, synthetic construct
  source 1..23: mol_type = other DNA
atagctgctggcagtataactgg

SEQ ID NO: 929 - DNA, 23 bp, synthetic construct
  source 1..23: mol_type = other DNA
gcatcacaacattgacactgtgg

SEQ ID NO: 930 - DNA, 23 bp, synthetic construct
  source 1..23: mol_type = other DNA
aaggcgagtttctacaaagatgg

SEQ ID NO: 931 - DNA, 23 bp, synthetic construct
  source 1..23: mol_type = other DNA
ttacgttatgactaatctttggg

SEQ ID NO: 932 - DNA, 23 bp, synthetic construct
  source 1..23: mol_type = other DNA
caagactgattaagactgatggg

SEQ ID NO: 933 - DNA, 23 bp, synthetic construct
  source 1..23: mol_type = other DNA
agcagcaatgtattaaaggctgg

SEQ ID NO: 934 - DNA, 23 bp, synthetic construct
  source 1..23: mol_type = other DNA
ctacaggattgatgtaaacatgg

SEQ ID NO: 935 - DNA, 23 bp, synthetic construct
  source 1..23: mol_type = other DNA
tgggcataaagggttttaatggg

SEQ ID NO: 936 - DNA, 23 bp, synthetic construct
  source 1..23: mol_type = other DNA
acatcaatcctgtaggtgattgg

SEQ ID NO: 937 - DNA, 23 bp, synthetic construct
  source 1..23: mol_type = other DNA
attctagtcattatagctgctgg

SEQ ID NO: 938 - DNA, 23 bp, synthetic construct
  source 1..23: mol_type = other DNA
catcaatcctgtaggtgattggg

SEQ ID NO: 939 - DNA, 23 bp, synthetic construct
  source 1..23: mol_type = other DNA
gttataagatcaattctgagtgg

SEQ ID NO: 940 - DNA, 23 bp, synthetic construct
  source 1..23: mol_type = other DNA
ggcagactgtggatcaaaagtgg

SEQ ID NO: 941 - DNA, 23 bp, synthetic construct
  source 1..23: mol_type = other DNA
atggctgcccaatcacctacagg

SEQ ID NO: 942 - DNA, 23 bp, synthetic construct
  source 1..23: mol_type = other DNA
tcaaagcatgtacttagagttgg

SEQ ID NO: 943 - DNA, 20 bp, synthetic construct
  source 1..20: mol_type = other DNA
tcgcccctcaaatcttacag

SEQ ID NO: 944 - DNA, 20 bp, synthetic construct
  source 1..20: mol_type = other DNA
tcaaatcttacagctgctca

SEQ ID NO: 945 - DNA, 20 bp, synthetic construct
  source 1..20: mol_type = other DNA
tcttacagctgctcactccc

SEQ ID NO: 946 - DNA, 20 bp, synthetic construct
  source 1..20: mol_type = other DNA
tacagctgctcactcccctg

SEQ ID NO: 947 - DNA, 20 bp, synthetic construct
  source 1..20: mol_type = other DNA
tgctcactcccctgcagggc

SEQ ID NO: 948 - DNA, 20 bp, synthetic construct
  source 1..20: mol_type = other DNA
tcccctgcagggcaacgccc

SEQ ID NO: 949 - DNA, 20 bp, synthetic construct
  source 1..20: mol_type = other DNA
tgcagggcaacgcccaggga

SEQ ID NO: 950 - DNA, 20 bp, synthetic construct
  source 1..20: mol_type = other DNA
tctcgattatgggcgggatt

SEQ ID NO: 951 - DNA, 20 bp, synthetic construct
  source 1..20: mol_type = other DNA
tcgcttctcgattatgggcg

SEQ ID NO: 952 - DNA, 20 bp, synthetic construct
  source 1..20: mol_type = other DNA
tgtcgagtcgcttctcgatt

SEQ ID NO: 953 - DNA, 20 bp, synthetic construct
  source 1..20: mol_type = other DNA
tccatgtcgagtcgcttctc

SEQ ID NO: 954 - DNA, 20 bp, synthetic construct
  source 1..20: mol_type = other DNA
tcgcctccatgtcgagtcgc

SEQ ID NO: 955 - DNA, 20 bp, synthetic construct
  source 1..20: mol_type = other DNA
tcgtcatcgcctccatgtcg

SEQ ID NO: 956 - DNA, 20 bp, synthetic construct
  source 1..20: mol_type = other DNA
tgatctcgtcatcgcctcca

SEQ ID NO: 957 - DNA, 20 bp, synthetic construct
  source 1..20: mol_type = other DNA
tggccggcctgaccactggg

SEQ ID NO: 958 - DNA, 20 bp, synthetic construct
  source 1..20: mol_type = other DNA
tgaaggcctggccggcctga

SEQ ID NO: 959 - DNA, 20 bp, synthetic construct
  source 1..20: mol_type = other DNA
tgagcactgaaggcctggcc

SEQ ID NO: 960 - DNA, 20 bp, synthetic construct
  source 1..20: mol_type = other DNA
tccactgagcactgaaggcc

SEQ ID NO: 961 - DNA, 20 bp, synthetic construct
  source 1..20: mol_type = other DNA
tggtttccactgagcactga

SEQ ID NO: 962 - DNA, 20 bp, synthetic construct
  source 1..20: mol_type = other DNA
tggggaaaatgacccaacag

SEQ ID NO: 963 - DNA, 20 bp, synthetic construct
  source 1..20: mol_type = other DNA
taggacagtggggaaaatga

SEQ ID NO: 964 - DNA, 20 bp, synthetic construct
  source 1..20: mol_type = other DNA
tccagggacacggtgctagg

SEQ ID NO: 965 - DNA, 20 bp, synthetic construct
  source 1..20: mol_type = other DNA
tcagagccaggagtcctggc

SEQ ID NO: 966 - DNA, 20 bp, synthetic construct
  source 1..20: mol_type = other DNA
tccttcagagccaggagtcc

SEQ ID NO: 967 - DNA, 20 bp, synthetic construct
  source 1..20: mol_type = other DNA
tcctccttcagagccaggag

SEQ ID NO: 968 - DNA, 20 bp, synthetic construct
  source 1..20: mol_type = other DNA
tccagcccctcctccttcag

SEQ ID NO: 969 - DNA, 20 bp, synthetic construct
  source 1..20: mol_type = other DNA
tccgagcttgacccttggaa

SEQ ID NO: 970 - DNA, 19 bp, synthetic construct
  source 1..19: mol_type = other DNA
tggtttccgagcttgaccc

SEQ ID NO: 971 - DNA, 20 bp, synthetic construct
  source 1..20: mol_type = other DNA
tggggtggtttccgagcttg

SEQ ID NO: 972 - DNA, 20 bp, synthetic construct
  source 1..20: mol_type = other DNA
tctgctggggtggtttccga

SEQ ID NO: 973 - DNA, 20 bp, synthetic construct
  source 1..20: mol_type = other DNA
tgcagagtatctgctggggt

SEQ ID NO: 974 - DNA, 18 bp, synthetic construct
  source 1..18: mol_type = other DNA
tcttcctagtattaaagt

SEQ ID NO: 975 - DNA, 17 bp, synthetic construct
  source 1..17: mol_type = other DNA
tccttaatattaccagt

SEQ ID NO: 976 - DNA, 25 bp, synthetic construct
  source 1..25: mol_type = other DNA
taccaagctgaaatgacacaaaagt

SEQ ID NO: 977 - DNA, 25 bp, synthetic construct
  source 1..25: mol_type = other DNA
tggctgtgtcacataccagcagaat

SEQ ID NO: 978 - DNA, 22 bp, synthetic construct
  source 1..22: mol_type = other DNA
tgttaatttgaatacaatcact

SEQ ID NO: 979 - DNA, 21 bp, synthetic construct
  source 1..21: mol_type = other DNA
tgtgtcacataccagcagaat

SEQ ID NO: 980 - DNA, 16 bp, synthetic construct
  source 1..16: mol_type = other DNA
tggtaactactaattt

SEQ ID NO: 981 - DNA, 19 bp, synthetic construct
  source 1..19: mol_type = other DNA
tgtcacataccagcagaat

SEQ ID NO: 982 - DNA, 21 bp, synthetic construct
  source 1..21: mol_type = other DNA
tgtgacacagccatcaacaat

SEQ ID NO: 983 - DNA, 16 bp, synthetic construct
  source 1..16: mol_type = other DNA
tcctttgatgaacagt

SEQ ID NO: 984 - DNA, 29 bp, synthetic construct
  source 1..29: mol_type = other DNA
tgtgtgcaatagcgttaaaggaactacat

SEQ ID NO: 985 - DNA, 17 bp, synthetic construct
  source 1..17: mol_type = other DNA
tctttcaatagcccact

SEQ ID NO: 986 - DNA, 22 bp, synthetic construct
  source 1..22: mol_type = other DNA
tctcaaatgacaagagcacagt

SEQ ID NO: 987 - DNA, 18 bp, synthetic construct
  source 1..18: mol_type = other DNA
taccagttaattagcact

SEQ ID NO: 988 - DNA, 18 bp, synthetic construct
  source 1..18: mol_type = other DNA
tgttgtgacctaagccat

SEQ ID NO: 989 - DNA, 23 bp, synthetic construct
  source 1..23: mol_type = other DNA
tctcatgttttaaagtcaagaat

SEQ ID NO: 990 - DNA, 19 bp, synthetic construct
  source 1..19: mol_type = other DNA
tcctgaattcagaacagat

SEQ ID NO: 991 - DNA, 26 bp, synthetic construct
  source 1..26: mol_type = other DNA
tagcatgatgtttcatgttgtgacct

SEQ ID NO: 992 - DNA, 25 bp, synthetic construct
  source 1..25: mol_type = other DNA
tgtttcatgttgtgacctaagccat

SEQ ID NO: 993 - DNA, 18 bp, synthetic construct
  source 1..18: mol_type = other DNA
tacaacagtctatttcat

SEQ ID NO: 994 - DNA, 26 bp, synthetic construct
  source 1..26: mol_type = other DNA
tcttcctagtctcttctctacccagt

SEQ ID NO: 995 - DNA, 25 bp, synthetic construct
  source 1..25: mol_type = other DNA
tacactccagcctgggaaacagagt

SEQ ID NO: 996 - DNA, 19 bp, synthetic construct
  source 1..19: mol_type = other DNA
tcttttccttaggacggct

SEQ ID NO: 997 - DNA, 17 bp, synthetic construct
  source 1..17: mol_type = other DNA
tcgctcaggcctgtcat

SEQ ID NO: 998 - DNA, 16 bp, synthetic construct
  source 1..16: mol_type = other DNA
tccatatggaagactt

SEQ ID NO: 999 - DNA, 18 bp, synthetic construct
  source 1..18: mol_type = other DNA
tacccagttaaccaccct

SEQ ID NO: 1000 - DNA, 27 bp, synthetic construct
  source 1..27: mol_type = other DNA
tggcgcatgcctgtaatcccagctact

SEQ ID NO: 1001 - DNA, 26 bp, synthetic construct
  source 1..26: mol_type = other DNA
tatacgaggagaaaattagcattcct

SEQ ID NO: 1002 - DNA, 23 bp, synthetic construct
  source 1..23: mol_type = other DNA
tctgcctcccaggttcacgcaat

SEQ ID NO: 1003 - DNA, 22 bp, synthetic construct
  source 1..22: mol_type = other DNA
tgccttgtcacgttttcacagt

SEQ ID NO: 1004 - DNA, 25 bp, synthetic construct
  source 1..25: mol_type = other DNA
tgtcaccttctgtatgtgcaaccat

SEQ ID NO: 1005 - DNA, 17 bp, synthetic construct
  source 1..17: mol_type = other DNA
tctgtatgtgcaaccat

SEQ ID NO: 1006 - DNA, 17 bp, synthetic construct
  source 1..17: mol_type = other DNA
tagtcaagcaacaggat

SEQ ID NO: 1007 - DNA, 18 bp, synthetic construct
  source 1..18: mol_type = other DNA
tccaagataattccccat

SEQ ID NO: 1008 - DNA, 16 bp, synthetic construct
  source 1..16: mol_type = other DNA
tctgcaagatcctttt

SEQ ID NO: 1009 - DNA, 29 bp, synthetic construct
  source 1..29: mol_type = other DNA
tgctatgtaaggtagcaaaaaggtaacct

SEQ ID NO: 1010 - DNA, 16 bp, synthetic construct
  source 1..16: mol_type = other DNA
tctctctcctcctgct

SEQ ID NO: 1011 - DNA, 19 bp, synthetic construct
  source 1..19: mol_type = other DNA
tccaaatgctattctctct

SEQ ID NO: 1012 - DNA, 17 bp, synthetic construct
  source 1..17: mol_type = other DNA
tgctgattcagcctcct

SEQ ID NO: 1013 - DNA, 21 bp, synthetic construct
  source 1..21: mol_type = other DNA
tagaacagccccccacacagt

SEQ ID NO: 1014 - DNA, 18 bp, synthetic construct
  source 1..18: mol_type = other DNA
tttagcagatgcatcagc

SEQ ID NO: 1015 - DNA, 20 bp, synthetic construct
  source 1..20: mol_type = other DNA
tgaccaggggcatgtcctgg

SEQ ID NO: 1016 - DNA, 21 bp, synthetic construct
  source 1..21: mol_type = other DNA
tggtccacctacctgaaaatg

SEQ ID NO: 1017 - DNA, 21 bp, synthetic construct
  source 1..21: mol_type = other DNA
tgtcccacaggtattacgggc

SEQ ID NO: 1018 - DNA, 21 bp, synthetic construct
  source 1..21: mol_type = other DNA
tacgggccaacctgacaatac

SEQ ID NO: 1019 - DNA, 21 bp, synthetic construct
  source 1..21: mol_type = other DNA
tgagctttggggactgaaaga

SEQ ID NO: 1020 - DNA, 21 bp, synthetic construct
  source 1..21: mol_type = other DNA
ctggcataatcttttccccca

SEQ ID NO: 1021 - DNA, 21 bp, synthetic construct
  source 1..21: mol_type = other DNA
ccagcctcctggccatgtgca

SEQ ID NO: 1022 - DNA, 21 bp, synthetic construct
  source 1..21: mol_type = other DNA
ggccatgtgcacaggggctga

SEQ ID NO: 1023 - DNA, 21 bp, synthetic construct
  source 1..21: mol_type = other DNA
ctgatatgtgaaggtttagca

SEQ ID NO: 1024 - DNA, 21 bp, synthetic construct
  source 1..21: mol_type = other DNA
tgaccaggcgtggtggctcac

SEQ ID NO: 1025 - DNA, 16 bp, synthetic construct
  source 1..16: mol_type = other DNA
tatagacattttcact

SEQ ID NO: 1026 - DNA, 21 bp, synthetic construct
  source 1..21: mol_type = other DNA
tctacatttaactatcaacct

SEQ ID NO: 1027 - DNA, 17 bp, synthetic construct
  source 1..17: mol_type = other DNA
tcgtgcaaacgtttgat

SEQ ID NO: 1028 - DNA, 18 bp, synthetic construct
  source 1..18: mol_type = other DNA
tacatcaatcctgtaggt

SEQ ID NO: 1029 - DNA, 20 bp, synthetic construct
  source 1..20: mol_type = other DNA
tctattttagtgacccaagt

SEQ ID NO: 1030 - DNA, 19 bp, synthetic construct
  source 1..19: mol_type = other DNA
tagagtcaaagcatgtact

SEQ ID NO: 1031 - DNA, 18 bp, synthetic construct
  source 1..18: mol_type = other DNA
tcctacccataagctcct

SEQ ID NO: 1032 - DNA, 17 bp, synthetic construct
  source 1..17: mol_type = other DNA
tccccatccccatcagt

SEQ ID NO: 1033 - DNA, 29 bp, synthetic construct
  source 1..29: mol_type = other DNA
tctttaattcaagcaagactttaacaagt

SEQ ID NO: 1034 - DNA, 16 bp, synthetic construct
  source 1..16: mol_type = other DNA
tgcagtcccctttctt

SEQ ID NO: 1035 - DNA, 20 bp, synthetic construct
  source 1..20: mol_type = other DNA
tctgcacaaatccccaaaga

SEQ ID NO: 1036 - DNA, 16 bp, synthetic construct
  source 1..16: mol_type = other DNA
tacatgctttgactct

SEQ ID NO: 1037 - DNA, 29 bp, synthetic construct
  source 1..29: mol_type = other DNA
tggccagttatactgccagcagctataat

SEQ ID NO: 1038 - DNA, 15 bp, synthetic construct
  source 1..15: mol_type = other DNA
tgggaagataaacta

SEQ ID NO: 1039 - DNA, 18 bp, synthetic construct
  source 1..18: mol_type = other DNA
actcccctgcagggcaac

SEQ ID NO: 1040 - DNA, 18 bp, synthetic construct
  source 1..18: mol_type = other DNA
cccctgcagggcaacgcc

SEQ ID NO: 1041 - DNA, 18 bp, synthetic construct
  source 1..18: mol_type = other DNA
ctgcagggcaacgcccag

SEQ ID NO: 1042 - DNA, 18 bp, synthetic construct
  source 1..18: mol_type = other DNA
cagggcaacgcccaggga

SEQ ID NO: 1043 - DNA, 18 bp, synthetic construct
  source 1..18: mol_type = other DNA
ggcaacgcccagggacca

SEQ ID NO: 1044 - DNA, 18 bp, synthetic construct
  source 1..18: mol_type = other DNA
aacgcccagggaccaagt

SEQ ID NO: 1045 - DNA, 18 bp, synthetic construct
  source 1..18: mol_type = other DNA
gcccagggaccaagttag

SEQ ID NO: 1046 - DNA, 18 bp, synthetic construct
  source 1..18: mol_type = other DNA
cagggaccaagttagccc

SEQ ID NO: 1047 - DNA, 15 bp, synthetic construct
  source 1..15: mol_type = other DNA
gcctaggcaaaagaa

SEQ ID NO: 1048 - DNA, 18 bp, synthetic construct
  source 1..18: mol_type = other DNA
cgcgaggaggaaaggagg

SEQ ID NO: 1049 - DNA, 18 bp, synthetic construct
  source 1..18: mol_type = other DNA
gaggaggaaaggagggag

SEQ ID NO: 1050 - DNA, 18 bp, synthetic construct
  source 1..18: mol_type = other DNA
gaggaaaggagggagggc

SEQ ID NO: 1051 - protein, 176 aa, synthetic construct
LEPGEKPYKCPECGKSFSDSGNLRVHQRTHTGEKPYKCPECGKSFSDPGHLVRHQRTHTGEKPYKCPECGKSFSRADNLTEHQRTHTGEKPYKCPECGKSFSRNDALTEHQRTHTGEKPYKCPECGKSFSSKKHLAEHQRTHTGEKPYKCPECGKSFSTHLDLIRHQRTHTGKKTS

SEQ ID NO: 1052 - protein, 176 aa, synthetic construct
LEPGEKPYKCPECGKSFSDCRDLARHQRTHTGEKPYKCPECGKSFSDSGNLRVHQRTHTGEKPYKCPECGKSFSDPGHLVRHQRTHTGEKPYKCPECGKSFSRADNLTEHQRTHTGEKPYKCPECGKSFSRNDALTEHQRTHTGEKPYKCPECGKSFSSKKHLAEHQRTHTGKKTS

SEQ ID NO: 1053 - protein, 176 aa, synthetic construct
LEPGEKPYKCPECGKSFSRADNLTEHQRTHTGEKPYKCPECGKSFSDCRDLARHQRTHTGEKPYKCPECGKSFSDSGNLRVHQRTHTGEKPYKCPECGKSFSDPGHLVRHQRTHTGEKPYKCPECGKSFSRADNLTEHQRTHTGEKPYKCPECGKSFSRNDALTEHQRTHTGKKTS

SEQ ID NO: 1054 - protein, 176 aa, synthetic construct
LEPGEKPYKCPECGKSFSQRAHLERHQRTHTGEKPYKCPECGKSFSRADNLTEHQRTHTGEKPYKCPECGKSFSDCRDLARHQRTHTGEKPYKCPECGKSFSDSGNLRVHQRTHTGEKPYKCPECGKSFSDPGHLVRHQRTHTGEKPYKCPECGKSFSRADNLTEHQRTHTGKKTS

SEQ ID NO: 1055 - protein, 176 aa, synthetic construct
LEPGEKPYKCPECGKSFSTSHSLTEHQRTHTGEKPYKCPECGKSFSQRAHLERHQRTHTGEKPYKCPECGKSFSRADNLTEHQRTHTGEKPYKCPECGKSFSDCRDLARHQRTHTGEKPYKCPECGKSFSDSGNLRVHQRTHTGEKPYKCPECGKSFSDPGHLVRHQRTHTGKKTS

SEQ ID NO: 1056 - protein, 176 aa, synthetic construct
LEPGEKPYKCPECGKSFSHRTTLTNHQRTHTGEKPYKCPECGKSFSTSHSLTEHQRTHTGEKPYKCPECGKSFSQRAHLERHQRTHTGEKPYKCPECGKSFSRADNLTEHQRTHTGEKPYKCPECGKSFSDCRDLARHQRTHTGEKPYKCPECGKSFSDSGNLRVHQRTHTGKKTS

SEQ ID NO: 1057 - protein, 176 aa, synthetic construct
LEPGEKPYKCPECGKSFSREDNLHTHQRTHTGEKPYKCPECGKSFSHRTTLTNHQRTHTGEKPYKCPECGKSFSTSHSLTEHQRTHTGEKPYKCPECGKSFSQRAHLERHQRTHTGEKPYKCPECGKSFSRADNLTEHQRTHTGEKPYKCPECGKSFSDCRDLARHQRTHTGKKTS

SEQ ID NO: 1058 - protein, 176 aa, synthetic construct
LEPGEKPYKCPECGKSFSSKKHLAEHQRTHTGEKPYKCPECGKSFSREDNLHTHQRTHTGEKPYKCPECGKSFSHRTTLTNHQRTHTGEKPYKCPECGKSFSTSHSLTEHQRTHTGEKPYKCPECGKSFSQRAHLERHQRTHTGEKPYKCPECGKSFSRADNLTEHQRTHTGKKTS

SEQ ID NO: 1059 - protein, 148 aa, synthetic construct
LEPGEKPYKCPECGKSFSQSSNLVRHQRTHTGEKPYKCPECGKSFSQRANLRAHQRTHTGEKPYKCPECGKSFSQSGDLRRHQRTHTGEKPYKCPECGKSFSREDNLHTHQRTHTGEKPYKCPECGKSFSDCRDLARHQRTHTGKKTS

SEQ ID NO: 1060 - protein, 176 aa, synthetic construct
LEPGEKPYKCPECGKSFSRSDHLTNHQRTHTGEKPYKCPECGKSFSRSDHLTNHQRTHTGEKPYKCPECGKSFSQSSNLVRHQRTHTGEKPYKCPECGKSFSRSDNLVRHQRTHTGEKPYKCPECGKSFSRSDNLVRHQRTHTGEKPYKCPECGKSFSHTGHLLEHQRTHTGKKTS

SEQ ID NO: 1061 - protein, 176 aa, synthetic construct
LEPGEKPYKCPECGKSFSRSDNLVRHQRTHTGEKPYKCPECGKSFSRSDHLTNHQRTHTGEKPYKCPECGKSFSRSDHLTNHQRTHTGEKPYKCPECGKSFSQSSNLVRHQRTHTGEKPYKCPECGKSFSRSDNLVRHQRTHTGEKPYKCPECGKSFSRSDNLVRHQRTHTGKKTS

SEQ ID NO: 1062 - protein, 176 aa, synthetic construct
LEPGEKPYKCPECGKSFSDPGHLVRHQRTHTGEKPYKCPECGKSFSRSDNLVRHQRTHTGEKPYKCPECGKSFSRSDHLTNHQRTHTGEKPYKCPECGKSFSRSDHLTNHQRTHTGEKPYKCPECGKSFSQSSNLVRHQRTHTGEKPYKCPECGKSFSRSDNLVRHQRTHTGKKTS

SEQ ID NO: 1063 - DNA, 30 bp, synthetic construct
  source 1..30: mol_type = other DNA
taggacagtggggaaaatgacccaacagcc

SEQ ID NO: 1064 - DNA, 21 bp, synthetic construct
  source 1..21: mol_type = other DNA
agagggagccacgaaaacaga

SEQ ID NO: 1065 - DNA, 15 bp, synthetic construct
  source 1..15: mol_type = other DNA
gcagatagccaggag

SEQ ID NO: 1066 - DNA, 18 bp, synthetic construct
  source 1..18: mol_type = other DNA
agatagccaggagtcctt

SEQ ID NO: 1067 - DNA, 24 bp, synthetic construct
  source 1..24: mol_type = other DNA
cccagtggtcaggccggccaggcc

SEQ ID NO: 1068 - DNA, 18 bp, synthetic construct
  source 1..18: mol_type = other DNA
ggccggccaggccttcag

SEQ ID NO: 1069 - DNA, 27 bp, synthetic construct
  source 1..27: mol_type = other DNA
agtgctcagtggaaaccacgaaaggac

SEQ ID NO: 1070 - protein, 21 aa, synthetic construct
TGGCCCCCAGCCCCTCCTGCC

SEQ ID NO: 1071 - DNA, 27 bp, synthetic construct
  source 1..27: mol_type = other DNA
agagccaggagtcctggcccccagccc

SEQ ID NO: 1072 - DNA, 24 bp, synthetic construct
  source 1..24: mol_type = other DNA
gcaggaggggctgggggccaggac

SEQ ID NO: 1073 - DNA, 24 bp, synthetic construct
  source 1..24: mol_type = other DNA
atagccctgggcccacggcttcgt

SEQ ID NO: 1074 - DNA, 15 bp, synthetic construct
  source 1..15: mol_type = other DNA
gaaggacctggctgg

SEQ ID NO: 1075 - DNA, 27 bp, synthetic construct
  source 1..27: mol_type = other DNA
gcaggaacgaagccgtgggcccagggc

SEQ ID NO: 1076 - DNA, 18 bp, synthetic construct
  source 1..18: mol_type = other DNA
ggaaaccaccccagcaga

SEQ ID NO: 1077 - DNA, 33 bp, synthetic construct
  source 1..33: mol_type = other DNA
aagggtcaagctcggaaaccaccccagcagata

SEQ ID NO: 1078 - protein, 288 aa, synthetic construct
LEPGEKPYKCPECGKSFSDCRDLARHQRTHTGEKPYKCPECGKSFSSPADLTRHQRTHTGEKPYKCPECGKSFSTSHSLTEHQRTHTGEKPYKCPECGKSFSDPGNLVRHQRTHTGEKPYKCPECGKSFSTTGNLTVHQRTHTGEKPYKCPECGKSFSQSSNLVRHQRTHTGEKPYKCPECGKSFSRSDKLVRHQRTHTGEKPYKCPECGKSFSHRTTLTNHQRTHTGEKPYKCPECGKSFSDPGNLVRHQRTHTGEKPYKCPECGKSFSREDNLHTHQRTHTGKKTS

SEQ ID NO: 1079 - protein, 204 aa, synthetic construct
LEPGEKPYKCPECGKSFSQLAHLRAHQRTHTGEKPYKCPECGKSFSDSGNLRVHQRTHTGEKPYKCPECGKSFSQSSNLVRHQRTHTGEKPYKCPECGKSFSSKKALTEHQRTHTGEKPYKCPECGKSFSERSHLREHQRTHTGEKPYKCPECGKSFSRSDKLVRHQRTHTGEKPYKCPECGKSFSQLAHLRAHQRTHTGKKTS

SEQ ID NO: 1080 - protein, 148 aa, synthetic construct
LEPGEKPYKCPECGKSFSRSDNLVRHQRTHTGEKPYKCPECGKSFSRADNLTEHQRTHTGEKPYKCPECGKSFSERSHLREHQRTHTGEKPYKCPECGKSFSTSGNLVRHQRTHTGEKPYKCPECGKSFSQSGDLRRHQRTHTGKKTS

SEQ ID NO: 1081 - protein, 176 aa, synthetic construct
LEPGEKPYKCPECGKSFSTTGALTEHQRTHTGEKPYKCPECGKSFSDPGALVRHQRTHTGEKPYKCPECGKSFSQRAHLERHQRTHTGEKPYKCPECGKSFSTSHSLTEHQRTHTGEKPYKCPECGKSFSREDNLHTHQRTHTGEKPYKCPECGKSFSQLAHLRAHQRTHTGKKTS

SEQ ID NO: 1082 - protein, 232 aa, synthetic construct
LEPGEKPYKCPECGKSFSDCRDLARHQRTHTGEKPYKCPECGKSFSRADNLTEHQRTHTGEKPYKCPECGKSFSDPGHLVRHQRTHTGEKPYKCPECGKSFSDCRDLARHQRTHTGEKPYKCPECGKSFSRADNLTEHQRTHTGEKPYKCPECGKSFSTSGHLVRHQRTHTGEKPYKCPECGKSFSHRTTLTNHQRTHTGEKPYKCPECGKSFSSKKHLAEHQRTHTGKKTS

SEQ ID NO: 1083 - protein, 176 aa, synthetic construct
LEPGEKPYKCPECGKSFSRADNLTEHQRTHTGEKPYKCPECGKSFSTTGALTEHQRTHTGEKPYKCPECGKSFSDPGHLVRHQRTHTGEKPYKCPECGKSFSTSHSLTEHQRTHTGEKPYKCPECGKSFSRSDKLTEHQRTHTGEKPYKCPECGKSFSDPGHLVRHQRTHTGKKTS

SEQ ID NO: 1084 - protein, 260 aa, synthetic construct
LEPGEKPYKCPECGKSFSDPGNLVRHQRTHTGEKPYKCPECGKSFSRKDNLKNHQRTHTGEKPYKCPECGKSFSQSGHLTEHQRTHTGEKPYKCPECGKSFSTSHSLTEHQRTHTGEKPYKCPECGKSFSQRANLRAHQRTHTGEKPYKCPECGKSFSRSDHLTTHQRTHTGEKPYKCPECGKSFSRADNLTEHQRTHTGEKPYKCPECGKSFSTSGELVRHQRTHTGEKPYKCPECGKSFSHRTTLTNHQRTHTGKKTS

SEQ ID NO: 1085 - protein, 204 aa, synthetic construct
LEPGEKPYKCPECGKSFSDCRDLARHQRTHTGEKPYKCPECGKSFSTKNSLTEHQRTHTGEKPYKCPECGKSFSTKNSLTEHQRTHTGEKPYKCPECGKSFSDCRDLARHQRTHTGEKPYKCPECGKSFSTSHSLTEHQRTHTGEKPYKCPECGKSFSSKKHLAEHQRTHTGEKPYKCPECGKSFSRSDHLTTHQRTHTGKKTS

SEQ ID NO: 1086 - protein, 260 aa, synthetic construct
LEPGEKPYKCPECGKSFSSKKHLAEHQRTHTGEKPYKCPECGKSFSRADNLTEHQRTHTGEKPYKCPECGKSFSSKKHLAEHQRTHTGEKPYKCPECGKSFSDPGHLVRHQRTHTGEKPYKCPECGKSFSTKNSLTEHQRTHTGEKPYKCPECGKSFSHRTTLTNHQRTHTGEKPYKCPECGKSFSRSDHLTNHQRTHTGEKPYKCPECGKSFSDCRDLARHQRTHTGEKPYKCPECGKSFSQLAHLRAHQRTHTGKKTS

SEQ ID NO: 1087 - protein, 232 aa, synthetic construct
LEPGEKPYKCPECGKSFSDPGNLVRHQRTHTGEKPYKCPECGKSFSRADNLTEHQRTHTGEKPYKCPECGKSFSDPGHLVRHQRTHTGEKPYKCPECGKSFSRSDKLVRHQRTHTGEKPYKCPECGKSFSTSGELVRHQRTHTGEKPYKCPECGKSFSRSDKLVRHQRTHTGEKPYKCPECGKSFSQRAHLERHQRTHTGEKPYKCPECGKSFSQSGDLRRHQRTHTGKKTS

SEQ ID NO: 1088 - protein, 231 aa, synthetic construct
LEPGEKPYKCPECGKSFSSRRTCRAHQRTHTGEKPYKCPECGKSFSTTGALTEHQRTHTGEKPYKCPECGKSFSRSDKLTEHQRTHTGEKPYKCPECGKSFSTSHSLTEHQRTHTGEKPYKCPECGKSFSDPGHLVRHQRTHTGEKPYKCPECGKSFSRNDALTEHQRTHTGEKPYKCPECGKSFSDCRDLARHQRTHTGEKPYKCPECGKSFSQKSSLIAHQRTHTGKKT

SEQ ID NO: 1089 - protein, 148 aa, synthetic construct
LEPGEKPYKCPECGKSFSRSDHLTTHQRTHTGEKPYKCPECGKSFSDPGHLVRHQRTHTGEKPYKCPECGKSFSTKNSLTEHQRTHTGEKPYKCPECGKSFSQRAHLERHQRTHTGEKPYKCPECGKSFSQSSNLVRHQRTHTGKKTS

SEQ ID NO: 1090 - protein, 260 aa, synthetic construct
LEPGEKPYKCPECGKSFSDPGHLVRHQRTHTGEKPYKCPECGKSFSRADNLTEHQRTHTGEKPYKCPECGKSFSDCRDLARHQRTHTGEKPYKCPECGKSFSRSDHLTTHQRTHTGEKPYKCPECGKSFSRNDTLTEHQRTHTGEKPYKCPECGKSFSRKDNLKNHQRTHTGEKPYKCPECGKSFSRTDTLRDHQRTHTGEKPYKCPECGKSFSQRAHLERHQRTHTGEKPYKCPECGKSFSQSGDLRRHQRTHTGKKTS

SEQ ID NO: 1091 - protein, 176 aa, synthetic construct
LEPGEKPYKCPECGKSFSQLAHLRAHQRTHTGEKPYKCPECGKSFSERSHLREHQRTHTGEKPYKCPECGKSFSSKKHLAEHQRTHTGEKPYKCPECGKSFSSKKALTEHQRTHTGEKPYKCPECGKSFSDSGNLRVHQRTHTGEKPYKCPECGKSFSQRAHLERHQRTHTGKKTS

SEQ ID NO: 1092 - protein, 316 aa, synthetic construct
LEPGEKPYKCPECGKSFSQKSSLIAHQRTHTGEKPYKCPECGKSFSRADNLTEHQRTHTGEKPYKCPECGKSFSRADNLTEHQRTHTGEKPYKCPECGKSFSSKKHLAEHQRTHTGEKPYKCPECGKSFSTSHSLTEHQRTHTGEKPYKCPECGKSFSQRANLRAHQRTHTGEKPYKCPECGKSFSRSDKLTEHQRTHTGEKPYKCPECGKSFSTSGELVRHQRTHTGEKPYKCPECGKSFSQSGNLTEHQRTHTGEKPYKCPECGKSFSTSGHLVRHQRTHTGEKPYKCPECGKSFSRKDNLKNHQRTHTGKKTS

SEQ ID NO: 1093 - DNA, 18 bp, synthetic construct
  source 1..18: mol_type = other DNA
ctttgatgaacagtcaca

SEQ ID NO: 1094 - DNA, 18 bp, synthetic construct
  source 1..18: mol_type = other DNA
cttccaattagtcctacc

SEQ ID NO: 1095 - DNA, 21 bp, synthetic construct
  source 1..21: mol_type = other DNA
atactaggaagaaatacaata

SEQ ID NO: 1096 - DNA, 18 bp, synthetic construct
  source 1..18: mol_type = other DNA
gctcttgtcatttgagat

SEQ ID NO: 1097 - DNA, 33 bp, synthetic construct
  source 1..33: mol_type = other DNA
ccaagctgaaatgacacaaaagttaaaacaaag

SEQ ID NO: 1098 - DNA, 21 bp, synthetic construct
  source 1..21: mol_type = other DNA
cttataccagttaattagcac

SEQ ID NO: 1099 - DNA, 24 bp, synthetic construct
  source 1..24: mol_type = other DNA
aacgctattgcacacatagttaca

SEQ ID NO: 1100 - DNA, 21 bp, synthetic construct
  source 1..21: mol_type = other DNA
tgaattcaggaacaaagtata

SEQ ID NO: 1101 - DNA, 27 bp, synthetic construct
  source 1..27: mol_type = other DNA
gctggtatgtgacacagccatcaacaa

SEQ ID NO: 1102 - protein, 176 aa, synthetic construct
LEPGEKPYKCPECGKSFSSPADLTRHQRTHTGEKPYKCPECGKSFSDPGALVRHQRTHTGEKPYKCPECGKSFSSPADLTRHQRTHTGEKPYKCPECGKSFSQAGHLASHQRTHTGEKPYKCPECGKSFSQAGHLASHQRTHTGEKPYKCPECGKSFSTTGALTEHQRTHTGKKTS

SEQ ID NO: 1103 - protein, 176 aa, synthetic construct
LEPGEKPYKCPECGKSFSDKKDLTRHQRTHTGEKPYKCPECGKSFSTKNSLTEHQRTHTGEKPYKCPECGKSFSHRTTLTNHQRTHTGEKPYKCPECGKSFSHKNALQNHQRTHTGEKPYKCPECGKSFSTSHSLTEHQRTHTGEKPYKCPECGKSFSTTGALTEHQRTHTGKKTS

SEQ ID NO: 1104 - protein, 204 aa, synthetic construct
LEPGEKPYKCPECGKSFSQKSSLIAHQRTHTGEKPYKCPECGKSFSSPADLTRHQRTHTGEKPYKCPECGKSFSTTGNLTVHQRTHTGEKPYKCPECGKSFSQLAHLRAHQRTHTGEKPYKCPECGKSFSQRAHLERHQRTHTGEKPYKCPECGKSFSQNSTLTEHQRTHTGEKPYKCPECGKSFSQKSSLIAHQRTHTGKKTS

SEQ ID NO: 1105 - protein, 176 aa, synthetic construct
LEPGEKPYKCPECGKSFSTSGNLVRHQRTHTGEKPYKCPECGKSFSQAGHLASHQRTHTGEKPYKCPECGKSFSHKNALQNHQRTHTGEKPYKCPECGKSFSDPGALVRHQRTHTGEKPYKCPECGKSFSTTGALTEHQRTHTGEKPYKCPECGKSFSTSGELVRHQRTHTGKKTS

SEQ ID NO: 1106 - protein, 316 aa, synthetic construct
LEPGEKPYKCPECGKSFSRKDNLKNHQRTHTGEKPYKCPECGKSFSSPADLTRHQRTHTGEKPYKCPECGKSFSQRANLRAHQRTHTGEKPYKCPECGKSFSTSGSLVRHQRTHTGEKPYKCPECGKSFSQRANLRAHQRTHTGEKPYKCPECGKSFSSPADLTRHQRTHTGEKPYKCPECGKSFSDPGNLVRHQRTHTGEKPYKCPECGKSFSTTGNLTVHQRTHTGEKPYKCPECGKSFSQAGHLASHQRTHTGEKPYKCPECGKSFSERSHLREHQRTHTGEKPYKCPECGKSFSTSHSLTEHQRTHTGKKTS

SEQ ID NO: 1107 - protein, 204 aa, synthetic construct
LEPGEKPYKCPECGKSFSSKKALTEHQRTHTGEKPYKCPECGKSFSREDNLHTHQRTHTGEKPYKCPECGKSFSTTGNLTVHQRTHTGEKPYKCPECGKSFSTSGSLVRHQRTHTGEKPYKCPECGKSFSTSHSLTEHQRTHTGEKPYKCPECGKSFSQKSSLIAHQRTHTGEKPYKCPECGKSFSTTGALTEHQRTHTGKKTS

SEQ ID NO: 1108 - protein, 232 aa, synthetic construct
LEPGEKPYKCPECGKSFSSPADLTRHQRTHTGEKPYKCPECGKSFSTSGSLVRHQRTHTGEKPYKCPECGKSFSQKSSLIAHQRTHTGEKPYKCPECGKSFSSKKALTEHQRTHTGEKPYKCPECGKSFSQSGDLRRHQRTHTGEKPYKCPECGKSFSHKNALQNHQRTHTGEKPYKCPECGKSFSTSGELVRHQRTHTGEKPYKCPECGKSFSDSGNLRVHQRTHTGKKTS

SEQ ID NO: 1109 - protein, 204 aa, synthetic construct
LEPGEKPYKCPECGKSFSQKSSLIAHQRTHTGEKPYKCPECGKSFSHRTTLTNHQRTHTGEKPYKCPECGKSFSQSGNLTEHQRTHTGEKPYKCPECGKSFSQSSNLVRHQRTHTGEKPYKCPECGKSFSRADNLTEHQRTHTGEKPYKCPECGKSFSHKNALQNHQRTHTGEKPYKCPECGKSFSQAGHLASHQRTHTGKKTS

SEQ ID NO: 1110 - protein, 260 aa, synthetic construct
LEPGEKPYKCPECGKSFSQSGNLTEHQRTHTGEKPYKCPECGKSFSQSGNLTEHQRTHTGEKPYKCPECGKSFSTSGNLTEHQRTHTGEKPYKCPECGKSFSERSHLREHQRTHTGEKPYKCPECGKSFSSKKALTEHQRTHTGEKPYKCPECGKSFSQAGHLASHQRTHTGEKPYKCPECGKSFSRRDELNVHQRTHTGEKPYKCPECGKSFSTSGHLVRHQRTHTGEKPYKCPECGKSFSTSGELVRHQRTHTGKKTS

SEQ ID NO: 1111 - DNA, 24 bp, synthetic construct
  source 1..24: mol_type = other DNA
cttcctgaaagcaagagatgaaat

SEQ ID NO: 1112 - DNA, 24 bp, synthetic construct
  source 1..24: mol_type = other DNA
ctgaaagcaagagatgaaattcca

SEQ ID NO: 1113 - DNA, 21 bp, synthetic construct
  source 1..21: mol_type = other DNA
atacgaggagaaaattagcat

SEQ ID NO: 1114 - DNA, 36 bp, synthetic construct
  source 1..36: mol_type = other DNA
catccatggcaggaagttgaagccaaaataaatctg

SEQ ID NO: 1115 - DNA, 27 bp, synthetic construct
  source 1..27: mol_type = other DNA
atggcaggaagttgaagccaaaataaa

SEQ ID NO: 1116 - DNA, 33 bp, synthetic construct
  source 1..33: mol_type = other DNA
gaaaagaagactcaaggaaacagagccaaacac

SEQ ID NO: 1117 - DNA, 24 bp, synthetic construct
  source 1..24: mol_type = other DNA
aggaaacagagccaaacacttaca

SEQ ID NO: 1118 - DNA, 60 bp, synthetic construct
  source 1..60: mol_type = other DNA
atgcagatttggacacagagtagtaaactgtgaaaacgtgacaaggcaaagtggcgtggg

SEQ ID NO: 1119 - DNA, 18 bp, synthetic construct
  source 1..18: mol_type = other DNA
ggacacagagtagtaaac

SEQ ID NO: 1120 - DNA, 18 bp, synthetic construct
  source 1..18: mol_type = other DNA
aaagctagcagcatggca

SEQ ID NO: 1121 - DNA, 24 bp, synthetic construct
  source 1..24: mol_type = other DNA
cctcttataaggcccaagaggata

SEQ ID NO: 1122 - DNA, 21 bp, synthetic construct
  source 1..21: mol_type = other DNA
caacatccttgacttaatcac

SEQ ID NO: 1123 - DNA, 18 bp, synthetic construct
  source 1..18: mol_type = other DNA
ggtagcaaaaaggtaacc

SEQ ID NO: 1124 - DNA, 36 bp, synthetic construct
  source 1..36: mol_type = other DNA
tggggtgcaagaggccaggccagagttgttctggtc

SEQ ID NO: 1125 - DNA, 24 bp, synthetic construct
  source 1..24: mol_type = other DNA
cgcatgctgattcagcctcctgac

SEQ ID NO: 1126 - DNA, 18 bp, synthetic construct
  source 1..18: mol_type = other DNA
agtcaagcaacaggatga

SEQ ID NO: 1127 - DNA, 30 bp, synthetic construct
  source 1..30: mol_type = other DNA
gtcaagcaacaggatgatccaaatgctatt

SEQ ID NO: 1128 - protein, 232 aa, synthetic construct
LEPGEKPYKCPECGKSFSTTGNLTVHQRTHTGEKPYKCPECGKSFSQAGHLASHQRTHTGEKPYKCPECGKSFSQLAHLRAHQRTHTGEKPYKCPECGKSFSRKDNLKNHQRTHTGEKPYKCPECGKSFSERSHLREHQRTHTGEKPYKCPECGKSFSQSSNLVRHQRTHTGEKPYKCPECGKSFSTKNSLTEHQRTHTGEKPYKCPECGKSFSTTGALTEHQRTHTGKKTS

SEQ ID NO: 1129 - protein, 232 aa, synthetic construct
LEPGEKPYKCPECGKSFSTSHSLTEHQRTHTGEKPYKCPECGKSFSHKNALQNHQRTHTGEKPYKCPECGKSFSQSSNLVRHQRTHTGEKPYKCPECGKSFSTSGNLVRHQRTHTGEKPYKCPECGKSFSQLAHLRAHQRTHTGEKPYKCPECGKSFSQSGDLRRHQRTHTGEKPYKCPECGKSFSQRANLRAHQRTHTGEKPYKCPECGKSFSRNDALTEHQRTHTGKKTS

SEQ ID NO: 1130 - protein, 204 aa, synthetic construct
LEPGEKPYKCPECGKSFSTSGNLTEHQRTHTGEKPYKCPECGKSFSREDNLHTHQRTHTGEKPYKCPECGKSFSTTGNLTVHQRTHTGEKPYKCPECGKSFSQSSNLVRHQRTHTGEKPYKCPECGKSFSQRAHLERHQRTHTGEKPYKCPECGKSFSQSGHLTEHQRTHTGEKPYKCPECGKSFSQKSSLIAHQRTHTGKKTS

SEQ ID NO: 1131 - protein, 344 aa, synthetic construct
LEPGEKPYKCPECGKSFSRNDALTEHQRTHTGEKPYKCPECGKSFSTTGNLTVHQRTHTGEKPYKCPECGKSFSQKSSLIAHQRTHTGEKPYKCPECGKSFSQRANLRAHQRTHTGEKPYKCPECGKSFSDCRDLARHQRTHTGEKPYKCPECGKSFSQSSNLVRHQRTHTGEKPYKCPECGKSFSTSGSLVRHQRTHTGEKPYKCPECGKSFSQSSNLVRHQRTHTGEKPYKCPECGKSFSRADNLTEHQRTHTGEKPYKCPECGKSFSRSDHLTTHQRTHTGEKPYKCPECGKSFSTSHSLTEHQRTHTGEKPYKCPECGKSFSTSGNLTEHQRTHTGKKTS

SEQ ID NO: 1132 - protein, 260 aa, synthetic construct
LEPGEKPYKCPECGKSFSQRANLRAHQRTHTGEKPYKCPECGKSFSTTGNLTVHQRTHTGEKPYKCPECGKSFSQSGNLTEHQRTHTGEKPYKCPECGKSFSERSHLREHQRTHTGEKPYKCPECGKSFSQAGHLASHQRTHTGEKPYKCPECGKSFSHRTTLTNHQRTHTGEKPYKCPECGKSFSQRAHLERHQRTHTGEKPYKCPECGKSFSQSGDLRRHQRTHTGEKPYKCPECGKSFSRRDELNVHQRTHTGKKTS

SEQ ID NO: 1133 - protein, 316 aa, synthetic construct
LEPGEKPYKCPECGKSFSSKKALTEHQRTHTGEKPYKCPECGKSFSQRANLRAHQRTHTGEKPYKCPECGKSFSDCRDLARHQRTHTGEKPYKCPECGKSFSQLAHLRAHQRTHTGEKPYKCPECGKSFSDSGNLRVHQRTHTGEKPYKCPECGKSFSQRAHLERHQRTHTGEKPYKCPECGKSFSQSGNLTEHQRTHTGEKPYKCPECGKSFSTHLDLIRHQRTHTGEKPYKCPECGKSFSRKDNLKNHQRTHTGEKPYKCPECGKSFSRKDNLKNHQRTHTGEKPYKCPECGKSFSQSSNLVRHQRTHTGKKTS

SEQ ID NO: 1134 - protein, 232 aa, synthetic construct
LEPGEKPYKCPECGKSFSSPADLTRHQRTHTGEKPYKCPECGKSFSTTGALTEHQRTHTGEKPYKCPECGKSFSSPADLTRHQRTHTGEKPYKCPECGKSFSQSGNLTEHQRTHTGEKPYKCPECGKSFSERSHLREHQRTHTGEKPYKCPECGKSFSRADNLTEHQRTHTGEKPYKCPECGKSFSQRANLRAHQRTHTGEKPYKCPECGKSFSRSDHLTNHQRTHTGKKTS

SEQ ID NO: 1135 - protein, 568 aa, synthetic construct
LEPGEKPYKCPECGKSFSRSDKLVRHQRTHTGEKPYKCPECGKSFSSRRTCRAHQRTHTGEKPYKCPECGKSFSRSDHLTTHQRTHTGEKPYKCPECGKSFSRKDNLKNHQRTHTGEKPYKCPECGKSFSQSGDLRRHQRTHTGEKPYKCPECGKSFSRKDNLKNHQRTHTGEKPYKCPECGKSFSDPGNLVRHQRTHTGEKPYKCPECGKSFSSRRTCRAHQRTHTGEKPYKCPECGKSFSQRANLRAHQRTHTGEKPYKCPECGKSFSQAGHLASHQRTHTGEKPYKCPECGKSFSRNDALTEHQRTHTGEKPYKCPECGKSFSQRANLRAHQRTHTGEKPYKCPECGKSFSHRTTLTNHQRTHTGEKPYKCPECGKSFSHRTTLTNHQRTHTGEKPYKCPECGKSFSRADNLTEHQRTHTGEKPYKCPECGKSFSSPADLTRHQRTHTGEKPYKCPECGKSFSRSDHLTTHQRTHTGEKPYKCPECGKSFSHKNALQNHQRTHTGEKPYKCPECGKSFSRADNLTEHQRTHTGEKPYKCPECGKSFSRRDELNVHQRTHTGKKTS

SEQ ID NO: 1136 - protein, 176 aa, synthetic construct
LEPGEKPYKCPECGKSFSDSGNLRVHQRTHTGEKPYKCPECGKSFSQSSSLVRHQRTHTGEKPYKCPECGKSFSQSSSLVRHQRTHTGEKPYKCPECGKSFSQLAHLRAHQRTHTGEKPYKCPECGKSFSSKKALTEHQRTHTGEKPYKCPECGKSFSQRAHLERHQRTHTGKKTS

SEQ ID NO: 1137 - protein, 176 aa, synthetic construct
LEPGEKPYKCPECGKSFSQSGDLRRHQRTHTGEKPYKCPECGKSFSRRDELNVHQRTHTGEKPYKCPECGKSFSERSHLREHQRTHTGEKPYKCPECGKSFSERSHLREHQRTHTGEKPYKCPECGKSFSTSGELVRHQRTHTGEKPYKCPECGKSFSQRANLRAHQRTHTGKKTS

SEQ ID NO: 1138 - protein, 232 aa, synthetic construct
LEPGEKPYKCPECGKSFSQKSSLIAHQRTHTGEKPYKCPECGKSFSRSDHLTNHQRTHTGEKPYKCPECGKSFSRKDNLKNHQRTHTGEKPYKCPECGKSFSSKKHLAEHQRTHTGEKPYKCPECGKSFSRSDHLTNHQRTHTGEKPYKCPECGKSFSQKSSLIAHQRTHTGEKPYKCPECGKSFSTTGALTEHQRTHTGEKPYKCPECGKSFSTKNSLTEHQRTHTGKKTS

SEQ ID NO: 1139 - protein, 204 aa, synthetic construct
LEPGEKPYKCPECGKSFSSKKALTEHQRTHTGEKPYKCPECGKSFSTTGNLTVHQRTHTGEKPYKCPECGKSFSTTGALTEHQRTHTGEKPYKCPECGKSFSQAGHLASHQRTHTGEKPYKCPECGKSFSTKNSLTEHQRTHTGEKPYKCPECGKSFSTSGNLTEHQRTHTGEKPYKCPECGKSFSQSGNLTEHQRTHTGKKTS

SEQ ID NO: 1140 - protein, 176 aa, synthetic construct
LEPGEKPYKCPECGKSFSDKKDLTRHQRTHTGEKPYKCPECGKSFSQSSSLVRHQRTHTGEKPYKCPECGKSFSRKDNLKNHQRTHTGEKPYKCPECGKSFSQRANLRAHQRTHTGEKPYKCPECGKSFSERSHLREHQRTHTGEKPYKCPECGKSFSTSGHLVRHQRTHTGKKTS

SEQ ID NO: 1141 - protein, 344 aa, synthetic construct
LEPGEKPYKCPECGKSFSDPGALVRHQRTHTGEKPYKCPECGKSFSRNDALTEHQRTHTGEKPYKCPECGKSFSTSGSLVRHQRTHTGEKPYKCPECGKSFSTSGSLVRHQRTHTGEKPYKCPECGKSFSQLAHLRAHQRTHTGEKPYKCPECGKSFSDCRDLARHQRTHTGEKPYKCPECGKSFSRADNLTEHQRTHTGEKPYKCPECGKSFSDPGHLVRHQRTHTGEKPYKCPECGKSFSQLAHLRAHQRTHTGEKPYKCPECGKSFSQSGDLRRHQRTHTGEKPYKCPECGKSFSTSGHLVRHQRTHTGEKPYKCPECGKSFSRSDHLTTHQRTHTGKKTS

SEQ ID NO: 1142 - protein, 232 aa, synthetic construct
LEPGEKPYKCPECGKSFSDPGNLVRHQRTHTGEKPYKCPECGKSFSTKNSLTEHQRTHTGEKPYKCPECGKSFSTKNSLTEHQRTHTGEKPYKCPECGKSFSRADNLTEHQRTHTGEKPYKCPECGKSFSHKNALQNHQRTHTGEKPYKCPECGKSFSRNDALTEHQRTHTGEKPYKCPECGKSFSRRDELNVHQRTHTGEKPYKCPECGKSFSHTGHLLEHQRTHTGKKTS

SEQ ID NO: 1143 - protein, 176 aa, synthetic construct
LEPGEKPYKCPECGKSFSQAGHLASHQRTHTGEKPYKCPECGKSFSQRAHLERHQRTHTGEKPYKCPECGKSFSSPADLTRHQRTHTGEKPYKCPECGKSFSQSGDLRRHQRTHTGEKPYKCPECGKSFSQSGNLTEHQRTHTGEKPYKCPECGKSFSHRTTLTNHQRTHTGKKTS

SEQ ID NO: 1144 - protein, 288 aa, synthetic construct
LEPGEKPYKCPECGKSFSHKNALQNHQRTHTGEKPYKCPECGKSFSTSGELVRHQRTHTGEKPYKCPECGKSFSTTGNLTVHQRTHTGEKPYKCPECGKSFSTSHSLTEHQRTHTGEKPYKCPECGKSFSTSGNLVRHQRTHTGEKPYKCPECGKSFSTSGNLVRHQRTHTGEKPYKCPECGKSFSRADNLTEHQRTHTGEKPYKCPECGKSFSQSGNLTEHQRTHTGEKPYKCPECGKSFSRKDNLKNHQRTHTGEKPYKCPECGKSFSDPGALVRHQRTHTGKKTS

SEQ ID NO: 1145 - DNA, 18 bp, synthetic construct
  source 1..18: mol_type = other DNA
gtagaaactcgccttatg

SEQ ID NO: 1146 - DNA, 21 bp, synthetic construct
  source 1..21: mol_type = other DNA
tgaatgagtcctgtccatctt

SEQ ID NO: 1147 - DNA, 21 bp, synthetic construct
  source 1..21: mol_type = other DNA
aagattagaacaaatgtccag

SEQ ID NO: 1148 - DNA, 18 bp, synthetic construct
  source 1..18: mol_type = other DNA
actctaagcagcaatgta

SEQ ID NO: 1149 - DNA, 18 bp, synthetic construct
  source 1..18: mol_type = other DNA
tgggatagtgaaaatgtc

SEQ ID NO: 1150 - DNA, 27 bp, synthetic construct
  source 1..27: mol_type = other DNA
aaaacttgggtcactaaaatagatgat

SEQ ID NO: 1151 - DNA, 27 bp, synthetic construct
  source 1..27: mol_type = other DNA
aaacatggaaaaggtcaaaaacttggg

SEQ ID NO: 1152 - DNA, 24 bp, synthetic construct
  source 1..24: mol_type = other DNA
aatgactagaatgaagtcctactg

SEQ ID NO: 1153 - protein, 176 aa, synthetic construct
LEPGEKPYKCPECGKSFSRRDELNVHQRTHTGEKPYKCPECGKSFSTTGALTEHQRTHTGEKPYKCPECGKSFSHTGHLLEHQRTHTGEKPYKCPECGKSFSTHLDLIRHQRTHTGEKPYKCPECGKSFSQSSNLVRHQRTHTGEKPYKCPECGKSFSQSSSLVRHQRTHTGKKTS

SEQ ID NO: 1154 - protein, 204 aa, synthetic construct
LEPGEKPYKCPECGKSFSTTGALTEHQRTHTGEKPYKCPECGKSFSTSGNLTEHQRTHTGEKPYKCPECGKSFSDPGALVRHQRTHTGEKPYKCPECGKSFSTKNSLTEHQRTHTGEKPYKCPECGKSFSHRTTLTNHQRTHTGEKPYKCPECGKSFSRRDELNVHQRTHTGEKPYKCPECGKSFSQAGHLASHQRTHTGKKTS

SEQ ID NO: 1155 - protein, 204 aa, synthetic construct
LEPGEKPYKCPECGKSFSRADNLTEHQRTHTGEKPYKCPECGKSFSDPGALVRHQRTHTGEKPYKCPECGKSFSTTGNLTVHQRTHTGEKPYKCPECGKSFSSPADLTRHQRTHTGEKPYKCPECGKSFSQLAHLRAHQRTHTGEKPYKCPECGKSFSHKNALQNHQRTHTGEKPYKCPECGKSFSRKDNLKNHQRTHTGKKTS

SEQ ID NO: 1156 - protein, 176 aa, synthetic construct
LEPGEKPYKCPECGKSFSQSSSLVRHQRTHTGEKPYKCPECGKSFSTTGNLTVHQRTHTGEKPYKCPECGKSFSERSHLREHQRTHTGEKPYKCPECGKSFSERSHLREHQRTHTGEKPYKCPECGKSFSQNSTLTEHQRTHTGEKPYKCPECGKSFSTHLDLIRHQRTHTGKKTS

SEQ ID NO: 1157 - protein, 176 aa, synthetic construct
LEPGEKPYKCPECGKSFSDPGALVRHQRTHTGEKPYKCPECGKSFSTTGNLTVHQRTHTGEKPYKCPECGKSFSQSSNLVRHQRTHTGEKPYKCPECGKSFSHRTTLTNHQRTHTGEKPYKCPECGKSFSTSGNLVRHQRTHTGEKPYKCPECGKSFSRSDHLTTHQRTHTGKKTS

SEQ ID NO: 1158 - protein, 260 aa, synthetic construct
LEPGEKPYKCPECGKSFSTSGNLVRHQRTHTGEKPYKCPECGKSFSTSGNLVRHQRTHTGEKPYKCPECGKSFSQKSSLIAHQRTHTGEKPYKCPECGKSFSQRANLRAHQRTHTGEKPYKCPECGKSFSTHLDLIRHQRTHTGEKPYKCPECGKSFSDPGALVRHQRTHTGEKPYKCPECGKSFSRSDHLTTHQRTHTGEKPYKCPECGKSFSTHLDLIRHQRTHTGEKPYKCPECGKSFSQRANLRAHQRTHTGKKTS

SEQ ID NO: 1159 - protein, 260 aa, synthetic construct
LEPGEKPYKCPECGKSFSRSDKLVRHQRTHTGEKPYKCPECGKSFSTTGALTEHQRTHTGEKPYKCPECGKSFSQRANLRAHQRTHTGEKPYKCPECGKSFSQSGNLTEHQRTHTGEKPYKCPECGKSFSTSGHLVRHQRTHTGEKPYKCPECGKSFSQRANLRAHQRTHTGEKPYKCPECGKSFSQRAHLERHQRTHTGEKPYKCPECGKSFSTSGNLTEHQRTHTGEKPYKCPECGKSFSQRANLRAHQRTHTGKKTS

SEQ ID NO: 1160 - protein, 232 aa, synthetic construct
LEPGEKPYKCPECGKSFSRNDALTEHQRTHTGEKPYKCPECGKSFSQNSTLTEHQRTHTGEKPYKCPECGKSFSDPGALVRHQRTHTGEKPYKCPECGKSFSQSSNLVRHQRTHTGEKPYKCPECGKSFSTTGNLTVHQRTHTGEKPYKCPECGKSFSREDNLHTHQRTHTGEKPYKCPECGKSFSDPGNLVRHQRTHTGEKPYKCPECGKSFSTTGNLTVHQRTHTGKKTS

SEQ ID NO: 1161 - DNA, 23 bp, synthetic construct
  source 1..23: mol_type = other DNA
ataacacgtgagccgtcctaagg

SEQ ID NO: 1162 - DNA, 77 bp, synthetic construct
  source 1..77: mol_type = other DNA
ttaacacttggattgcgggaaacgagttaagtcggctcgcgtgaattgcgcgtactccgcgggagccgtcttaactc

SEQ ID NO: 1163 - DNA, 132 bp, synthetic construct
  source 1..132: mol_type = other DNA
catttcaatcgaacccatacttcaaaagatataggcattttaaactaactctgattttgcgcgggaaacctaaataattgcccgcgccatcttatattttggcgggaaattcacccgacaccgtagtgttaa

SEQ ID NO: 1164-1165 - skipped (2 entries)

SEQ ID NO: 1166 - DNA, 51 bp, synthetic construct
  source 1..51: mol_type = other DNA
gccgcagtggagatgacttgtttgttaacggcactttaaacagcactgcgg

SEQ ID NO: 1167 - DNA, 25 bp, synthetic construct
  source 1..25: mol_type = other DNA
ttaccaccaagttaacactacggtg

SEQ ID NO: 1168 - DNA, 2055 bp, synthetic construct
  source 1..2055: mol_type = other DNA
atgccgaaaaaaaaacgaaaggtgtacccctacgatgtaccggactatgcaggaagcgcccagcacagcgactaccccgacgacgagttcagagccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggaggtctcgaaccaggcgagaagccttatgcctgtcctgagtgtggcaaatccttctcaagaaaagactctctggttagacaccagagaacacatacaggggagaaaccctataaatgccccgaatgcggaaagtccttttcccagagcggcgatctccggaggcatcagagaactcatacaggcgagaaaccatataagtgccccgagtgtgggaaatccttttccgattgtagagacctggccagacatcaaaggacacatacaggcaagaagaccggaggtggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcaaggccgccaagggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccaaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattac

SEQ ID NO: 1169 - DNA, 2055 bp, synthetic construct
  source 1..2055: mol_type = other DNA
atgccgaaaaaaaaacgaaaggtgtacccctacgatgtaccggactatgcaggaagcgcccagcacagcgactaccccgacgacgagttcagagccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggaggtctcgaaccaggcgagaagccttatgcctgtcctgagtgtggcaaatccttctcaagaaaagactctctggttagacaccagagaacacatacaggggagaaaccctataaatgccccgaatgcggaaagtccttttcccagagcggcgatctccggaggcatcagagaactcatacaggcgagaaaccatataagtgccccgagtgtgggaaatccttttccgattgtagagacctggccagacatcaaaggacacatacaggcaagaagaccggaggtggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcaagaaagccaagggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccaaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattac

SEQ ID NO: 1170 - DNA, 3243 bp, synthetic construct
  source 1..3243: mol_type = other DNA
atggcgcctaagaagaagcgcaaggtgggcggagcccagcacagcgactaccccgacgacgagttcagagccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggcggtggtgggtctgttgacctccggactttgggttactcccaacagcaacaagagaagattaaacctaaggtgcgctcaacagtggcgcagcatcatgaggcactggttggtcacgggttcacacatgcccatatagtagcactttcacaacatcccgctgcactcggaacggttgcagtaaagtaccaagacatgattgccgcattgccggaagcaactcatgaggccattgtcggagtaggcaaataccacggtgctagagctctcgaagcactcctcactgtggctggcgaattgagggggcccccccttcaacttgatactggacagctgttgaagatagcgaaacgagggggagtaaccgcagtcgaggccgtccacgcgtggcgaaatgcattgacgggagcgcctctcaaccttactccagatcaggttgtggccatagcgagcaacatcggaggtaagcaagcactggaaactgttcaacgattgctcccagtactctgccaagaccacggattgaccccagaacaggttgtggctattgccagcaacggcggcggtaaacaggctctggaaactgtgcagcgactcttgccggtgttgtgccaagctcatggccttacgccagaccaggttgttgctatcgctagcaacatcggaggcaaacaggcacttgaaacggtccaacgcctcttgccagtattgtgccaggcacatggcctcacccccgcccaggttgtggccatcgcaaacaataacgggggcaagcaggctttggagacagtccaacgattgttgccggtactttgtcaggaccacgggctgaccccagatcaagtcgtagcaatcgctagcaacatcggcgggaagcaggccctggaaacggtgcagaggctcctcccagtgctctgccaagatcatggcctcaccccagagcaagtggtcgcaatcgcaaacaataacggtggcaagcaagccctcgaaactgtgcaaaggttgctgccagttctctgtcaagcccacggtttgacaccggatcaggtagtggctatcgctagcaacggcggtggcaagcaagctctcgagacagtccagaggcttttgccagtactgtgccaagcgcacggtctcactcctgcgcaagttgttgctatagcaaacaataacggtggaaagcaagctcttgagactgtgcagcgcctgttgcccgtcctctgtcaggaccacggtttgactccagaccaagttgtcgccattgcaagcaacggcggtggacgccctgcactggagtccatcgtcgcacaactgtctaggccggaccccgccctggctgcccttactaatgatcacttggttgcattggcgtgtctgggcggtcggccagcgctggacgcagtcaagaagggactcccccacgcccctgcacttataaaacgcactaacaggagaataccagaacggacgagtcatcgagtcgctggagggggcggtagcggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattac

SEQ ID NO: 1171 - DNA, 1809 bp, synthetic construct
  source 1..1809: mol_type = other DNA
atgccgaaaaaaaaacgaaaggtgtacccctacgatgtaccggactatgcaggaagcgcccagcacagcgactaccccgacgacgagttcagagccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggctccagactggaagaggaactgagaagaaggctcacagaaggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcagaaaaaacagaggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccgaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattac

SEQ ID NO: 1172 - DNA, 1884 bp, synthetic construct
  source 1..1884: mol_type = other DNA
atggcgcctaagaagaagcgcaaggtgggcggagttgacctccggactttgggttactcccaacagcaacaagagaagattaaacctaaggtgcgctcaacagtggcgcagcatcatgaggcactggttggtcacgggttcacacatgcccatatagtagcactttcacaacatcccgctgcactcggaacggttgcagtaaagtaccaagacatgattgccgcattgccggaagcaactcatgaggccattgtcggagtaggcaaataccacggtgctagagctctcgaagcactcctcactgtggctggcgaattgagggggcccccccttcaacttgatactggacagctgttgaagatagcgaaacgagggggagtaaccgcagtcgaggccgtccacgcgtggcgaaatgcattgacgggagcgcctctcaaccttactccagatcaggttgtggccatagcgagcaacatcggaggtaagcaagcactggaaactgttcaacgattgctcccagtactctgccaagaccacggattgaccccagaacaggttgtggctattgccagcaacggcggcggtaaacaggctctggaaactgtgcagcgactcttgccggtgttgtgccaagctcatggccttacgccagaccaggttgttgctatcgctagcaacatcggaggcaaacaggcacttgaaacggtccaacgcctcttgccagtattgtgccaggcacatggcctcacccccgcccaggttgtggccatcgcaaacaataacgggggcaagcaggctttggagacagtccaacgattgttgccggtactttgtcaggaccacgggctgaccccagatcaagtcgtagcaatcgctagcaacatcggcgggaagcaggccctggaaacggtgcagaggctcctcccagtgctctgccaagatcatggcctcaccccagagcaagtggtcgcaatcgcaaacaataacggtggcaagcaagccctcgaaactgtgcaaaggttgctgccagttctctgtcaagcccacggtttgacaccggatcaggtagtggctatcgctagcaacggcggtggcaagcaagctctcgagacagtccagaggcttttgccagtactgtgccaagcgcacggtctcactcctgcgcaagttgttgctatagcaaacaataacggtggaaagcaagctcttgagactgtgcagcgcctgttgcccgtcctctgtcaggaccacggtttgactccagaccaagttgtcgccattgcaagcaacggcggtggacgccctgcactggagtccatcgtcgcacaactgtctaggccggaccccgccctggctgcccttactaatgatcacttggttgcattggcgtgtctgggcggtcggccagcgctggacgcagtcaagaagggactcccccacgcccctgcacttataaaacgcactaacaggagaataccagaacggacgagtcatcgagtcgctggagggggcggtagcgaagtccagctccaagaaagcggcgggggcctcgtgcagccaggcgggtccctgaggctgagctgtaccgcatctggagtcaccatctctgcccttaacgcaatggctatggggtggtatcggcaggcgcccggcgagaggagagtcatggtagcagccgttagcgaaagggggaatgcgatgtaccgagagagtgttcagggacgatttactgtcactcgggattttacgaacaaaatggtttcattgcaaatggacaatttgaaaccagaggacaccgctgtttactactgtcacgtcctggaagatcgagtagatagcttccatgactattggggccaaggaacacaagtgactgtcagctcc

SEQ ID NO: 1173 - DNA, 1809 bp, synthetic construct
  source 1..1809: mol_type = other DNA
atgccgaaaaaaaaacgaaaggtgtacccctacgatgtaccggactatgcaggaagcgcccagcacagcgactaccccgacgacgagttcagagccgataagctgagtaactacagctgcgacagcgacctggaaaacgccagcacatccgacgaggacagctctgacgacgaggtgatggtgcggcccagaaccctgagacggagaagaatcagcagctctagcagcgactctgaatccgacatcgagggctccagactggaagaggaactgagaagaaggctcacagaaggccgggaagagtggagccacgtggacaaccctcctgttctggaagattttctgggccatcagggcctgaacaccgacgccgtgatcaacaacatcgaggatgccgtgaagctgttcataggagatgatttctttgagttcctggtcgaggaatccaaccgctattacaaccagaatagaaacaacttcaagctgagcaagaaaagcctgaagtggaaggacatcacccctcaggagatgaaaaagttcctgggactgatcgttctgatgggacaggtgcggaaggacagaagggatgattactggacaaccgaaccttggaccgagaccccttactttggcaagaccatgaccagagacagattcagacagatctggaaagcctggcacttcaacaacaatgctgatatcgtgaacgagtctgatagactgtgtaaagtgcggccagtgttggattacttcgtgcctaagttcatcaacatctataagcctcaccagcagctgagcctggatgaaggcatcgtgccctggcggggcagactgttcttcagagtgtacaatgctggcaagatcgtcaaatacggcatcctggtgcgccttctgtgcgagagcgatacaggctacatctgtaatatggaaatctactgcggcgagggcaaaagactgctggaaaccatccagaccgtcgtttccccttataccgacagctggtaccacatctacatggacaactactacaattctgtggccaactgcgaggccctgatgaagaacaagtttagaatctgcggcacaatcaagaaagccaagggcatccctaaggacttccagaccatctctctgaagaagggcgaaaccaagttcatcagaaagaacgacatcctgctccaagtgtggcagtccaagaaacccgtgtacctgatcagcagcatccatagcgccgagatggaagaaagccagaacatcgacagaacaagcaagaagaagatcgtgaagcccaatgctctgatcgactacaacaagcacatgaaaggcgtggaccgggccaaccagtacctgtcttattactctatcctgagaagaacagtgaaatggaccaagagactggccatgtacatgatcaattgcgccctgttcaacagctacgccgtgtacaagtccgtgcgacaaagaaaaatgggattcaagatgttcctgaagcagacagccatccactggctgacagacgacattcctgaggacatggacattgtgccagatctgcaacctgtgcccagcacctctggtatgagagctaagcctcccaccagcgatcctccatgtagactgagcatggacatgcggaagcacaccctgcaggccatcgtcggcagcggcaagaagaagaacatccttagacggtgcagggtgtgcagcgtgcacaagctgcggagcgagactcggtacatgtgcaagttttgcaacattcccctgcacaagggagcctgcttcgagaagtaccacaccctgaagaattac

SEQ ID NO: 1174 - DNA, 1884 bp, synthetic construct
  source 1..1884: mol_type = other DNA
atggcgcctaagaagaagcgcaaggtgggcggagttgacctccggactttgggttactcccaacagcaacaagagaagattaaacctaaggtgcgctcaacagtggcgcagcatcatgaggcactggttggtcacgggttcacacatgcccatatagtagcactttcacaacatcccgctgcactcggaacggttgcagtaaagtaccaagacatgattgccgcattgccggaagcaactcatgaggccattgtcggagtaggcaaataccacggtgctagagctctcgaagcactcctcactgtggctggcgaattgagggggcccccccttcaacttgatactggacagctgttgaagatagcgaaacgagggggagtaaccgcagtcgaggccgtccacgcgtggcgaaatgcattgacgggagcgcctctcaaccttacgccggatcaagtcgtggcgatagctagtaacggaggtggtaaacaggcgctggagacggtacagcgcttgctgccagtgctctgccaagatcacggccttacgccagagcaagtggtggctattgcgaataataacggtggaaaacaggcgttggaaacagtccaaagactgctcccggtgttgtgccaagctcatggactgacgcccgatcaagtagtagctatcgcaagcaacggtggggggaaacaggcactggagactgtccagaggctcctccctgtgttgtgccaagcccatgggcttactccagctcaggttgtagcgatcgccagcaacattggaggaaagcaggccctggaaactgttcaacgcctcctcccagtattgtgccaagatcacggacttaccccggatcaagtcgtcgcaatcgcgtcccacgatggcgggaaacaggcacttgagaccgtgcaacgactgttgcccgtgctctgccaagaccatggcctcacacctgagcaagtagtggcgattgcgtctcacgatgggggcaagcaggcgcttgagaccgttcagcgattgcttcctgtcctctgccaggcccacgggttgacacctgatcaggtggtagccattgcgtctaacggtggaggtaagcaagcactcgagaccgttcaaaggctcttgccagtattgtgtcaggctcatggtcttacgccagcccaagttgtcgccatcgcatctcatgacggtgggaagcaggcacttgagactgtacaaagattgctcccagtgttgtgccaggaccacggcttgacgcctgatcaagttgtagcgattgcttcacatgatgggggcaggcctgcgttggagtccatcgtcgcacaactgtctaggccggaccccgccctggctgcccttactaatgatcacttggttgcattggcgtgtctgggcggtcggccagcgctggacgcagtcaagaagggactcccccacgcccctgcacttataaaacgcactaacaggagaataccagaacggacgagtcatcgagtcgctggagggggcggtagcgaagtccagctccaagaaagcggcgggggcctcgtgcagccaggcgggtccctgaggctgagctgtaccgcatctggagtcaccatctctgcccttaacgcaatggctatggggtggtatcggcaggcgcccggcgagaggagagtcatggtagcagccgttagcgaaagggggaatgcgatgtaccgagagagtgttcagggacgatttactgtcactcgggattttacgaacaaaatggtttcattgcaaatggacaatttgaaaccagaggacaccgctgtttactactgtcacgtcctggaagatcgagtagatagcttccatgactattggggccaaggaacacaagtgactgtcagctcc

SEQ ID NO: 1175 - DNA, 1884 bp, synthetic construct
  source 1..1884: mol_type = other DNA
atggcgcctaagaagaagcgcaaggtgggcggagttgacctccggactttgggttactcccaacagcaacaagagaagattaaacctaaggtgcgctcaacagtggcgcagcatcatgaggcactggttggtcacgggttcacacatgcccatatagtagcactttcacaacatcccgctgcactcggaacggttgcagtaaagtaccaagacatgattgccgcattgccggaagcaactcatgaggccattgtcggagtaggcaaataccacggtgctagagctctcgaagcactcctcactgtggctggcgaattgagggggcccccccttcaacttgatactggacagctgttgaagatagcgaaacgagggggagtaaccgcagtcgaggccgtccacgcgtggcgaaatgcattgacgggagcgcctctcaaccttacacctgatcaagtagttgccattgcgagtcatgatggtgggaaacaggccttggaaactgtccagcgccttctgcctgtgctttgtcaagatcatgggttgacacccgagcaagttgtagctattgcgtcacacgacggtggtaaacaggcgctggaaactgttcagcgcttgctcccagtcctctgtcaggcccatggtctcaccccggaccaggtagtagctatagctagcaatatcggaggaaagcaagcgttggaaaccgtgcagcggttgctccccgtcctgtgtcaagcacacggcttgacgccggcccaggtggtagccattgcgtctaacatcggcggcaagcaggcactcgagactgtgcaacggctcctgccggttctgtgccaagatcacggtcttactcctgatcaggtcgttgcgattgctagtaatggcggtggaaaacaggccctggagactgtccagcggcttctccctgtcctgtgccaggaccatggactcacaccagaacaggtagttgcaatagcctctaatataggtggcaagcaagcactcgagacagtccaaagacttcttcccgtcctttgccaggcgcatggtctcacccctgatcaagttgttgcgatagcgagtaacattggaggaaaacaggcattggaaacggtacaacggcttctcccggtcttgtgtcaagcgcatggcctcaccccggcccaagtggtcgctatagcatccaatattgggggtaagcaagcactggaaacggttcagcgacttttgcctgtgctgtgccaagaccacggacttacaccggaccaagttgtcgctatcgcctccaacgggggaggacggccggccttggagtccatcgtcgcacaactgtctaggccggaccccgccctggctgcccttactaatgatcacttggttgcattggcgtgtctgggcggtcggccagcgctggacgcagtcaagaagggactcccccacgcccctgcacttataaaacgcactaacaggagaataccagaacggacgagtcatcgagtcgctggagggggcggtagcgaagtccagctccaagaaagcggcgggggcctcgtgcagccaggcgggtccctgaggctgagctgtaccgcatctggagtcaccatctctgcccttaacgcaatggctatggggtggtatcggcaggcgcccggcgagaggagagtcatggtagcagccgttagcgaaagggggaatgcgatgtaccgagagagtgttcagggacgatttactgtcactcgggattttacgaacaaaatggtttcattgcaaatggacaatttgaaaccagaggacaccgctgtttactactgtcacgtcctggaagatcgagtagatagcttccatgactattggggccaaggaacacaagtgactgtcagctcc

SEQ ID NO: 1176 - DNA, 4068 bp, synthetic construct
  source 1..4068: mol_type = other DNA
ttaacactacggtgtcgggtgaatttcccgccaaaatataagatggcgcgggcaattatttaggtttcccgcgcaaaatcagagttagtttaaaatgcctatatcttttgaagtatgggttcgattgaaatgaaattttcggtttcttgtgtaaaaaatgaaaatttattcaatcacagacatttttttatctattcagtacataaataattctcgagctccccagcatgcctgctattgtcttcccaatcctccccctttccgctcacaattccacacaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtgaaacatgagagcttagtacgtactatcaacaggttgaactgccagatatcccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggctgctgtattatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggcgccaccatggtgtctaagggcgaagagctctttactggcgtggtgcccatcctggttgaattggacggagatgttaacggacacaaatttagcgtatctggagagggcgaaggtaagtaatcggttcaagtgattctcctgcctcagcctcccaagtagctgggattagaggtccccaccaccatgcctggctaattttttgtactttcagtagaaatggggttttgccatgttggccaggctgttctcgaactcctgagctcaggtgatccaactgtctcggcctcccaaagtgctgggattacaggcgtgagccactgtgcctagcctgagccaccacgccggcctaatttttaaattttttgtagagacagggtctcattatgttgcccagggtggtgtcaagctccaggtctcaagtgatccccctacctccgcctcccaaagttgtgggattgtaggcatgagccactgcaagaaaaccttaactgcagcctaataattgttttctttgggataacttttaaagtacattaaaagactatcaacttaatttctgatcatattttgttgaataaaataagtaaaatgtcttgtgaaacaaaatgctttttaacatccatataaagctatctatatatagctatctatgtctggcgcgcctaacaacaaatatttggtaatacgcagttacaatcggcccgagtttacacgtttcccgcactccaccgcaaatctatatgaaccgagttaagacggctcccgcggagtacgcgcaattcacgcgagccgacttaactcgtttcccgcaatccaagtgttaa

SEQ ID NO: 1177 - DNA, 4330 bp, synthetic construct
  source 1..4330: mol_type = other DNA
ttaacacttggattgcgggaaacgagttaagtcggctcgcgtgaattgcgcgtactccgcgggagccgtcttaactcggttcatatagatttgcggtggagtgcgggaaacgtgtaaactcgggccgattgtaactgcgtattaccaaatatttgttgtcgactctccctatagtgagtcgtattaattctcccagatcctctagcgataagcttgatatcgaattcgaggtcgacttctagagagctcaataaaagagcccacaacccctcactcggcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatggccttaccagtgaccgccttgctcctgccgctggccttgctgctccacgccgccaggccggacatccagatgacacagactacatcctccctgtctgcctctctgggagacagagtcaccatcagttgcagggcaagtcaggacatctctaagtatttgaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgagggcagaggcagcctgctgacatgtggcgacgtggaagagaaccctggccccatggttggttcgctaaactgcatcgtcgctgtgtcccagaacatgggcatcggcaagaacggggacttcccctggccaccgctcaggaatgaatccagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgattaatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcatttaggtgacactatagaagagactagtaattatttatgtactgaatagataaaaaaatgtctgtgattgaataaattttcattttttacacaagaaaccgaaaatttcatttcaatcgaacccatacttcaaaagatataggcattttaaactaactctgattttgcgcgggaaacctaaataattgcccgcgccatcttatattttggcgggaaattcacccgacaccgtagtgttaa

SEQ ID NO: 1178 - protein, 13 aa, synthetic construct
SRLEEELRRRLTE